(19) United States
(12) Patent Application Publication
(10) Pub. No.: US 2024/0150345 A1
(43) Pub. Date: May 9, 2024

(54) GCN2 MODULATING COMPOUNDS AND USES THEREOF

(71) Applicant: HiberCell, Inc., New York, NY (US)

(72) Inventors: Savithri Ramurthy, New York, NY (US); Mark J. Mulvihill, Sudbury, MA (US); Bradley Sherborne, Hoddesdon (GB); Benjamin Rahemtulla, Hoddesdon (GB); Eric P.A. Talbot, Hoddesdon (GB); Christopher G. Thomson, Hoddesdon (GB)

(21) Appl. No.: 18/262,489

(22) PCT Filed: Jan. 21, 2022
(86) PCT No.: PCT/US2022/013382

§ 371 Date: Jul. 21, 2023

Related U.S. Application Data
(60) Provisional application No. 63/140,311, filed on Jan. 22, 2021.

(51) Int. Cl. C07D 471/04; C07D 401/12; C07D 487/04; C07D 498/04

(52) U.S. Cl. CPC C07D 471/04; C07D 401/12; C07D 487/04; C07D 498/04

(57) ABSTRACT

Provided herein are compounds, compositions, and methods useful for modulating the activity of GCN2 and for treating related conditions, diseases, and disorders (e.g., cancer and neurodegenerative diseases).

### CROSS REFERENCE TO RELATED APPLICATION

This application claims priority to U.S. Provisional Application No. 63/140,311 filed Jan. 22, 2021, which is incorporated herein by reference in its entirety.

### BACKGROUND OF THE INVENTION

Cancer continues to be a significant health problem despite the substantial research efforts and scientific advances reported in the literature for treating this disease. Some of the most frequently diagnosed cancers include prostate cancer, breast cancer, and lung cancer. Prostate cancer is the most common form of cancer in men. Breast cancer remains a leading cause of death in women. Current treatment options for these cancers are not effective for all patients and/or can have substantial adverse side effects. New therapies are needed to address this unmet need in cancer therapy.

General control nonderepressible kinase 2 (GCN2) is a serine/threonine protein kinase that phosphorylates the a subunit of eukaryotic initiation factor 2 (eIF2α) in response to amino acid deficiency (see, for example, Wek, R. C. et al. in Biochem. Soc. Trans. 2006, 34 (Pt 1), p. 7-11). Expression and activation of GCN2 have been shown to be elevated in human and mouse tumors, and reduction in the expression of GCN2 has been shown to inhibit tumor growth (see e.g., Ye, J. et al. in EMBO J. 2010, 29(12), p. 2082-2096). Tumors grow in an environment of amino acid deficiency which can be further depleted with chemotherapy inducing a dependence on autophagy which requires GCN2 activity. In addition, GCN2 mediates the induction of anergy in T cells in response to tryptophan depletion by indoleamine 2,3-dioxygenase (IDO) in the tumor microenvironment (Munn, D. H. et al in Immunity 2005, 22, p. 633-642) and is essential for the proliferative fitness of cytotoxic T cells in amino acid limiting environments (Van de Velde, L-A., et al. in Cell Reports 2016, 17, p. 2247-2258). Inhibition of GCN2 has been reported as a therapeutic approach for cancer therapy (see, e.g., Wei, C. et al. in Mol. Biol. Cell. 2015, 26(6), p. 1044-1057). Accordingly, compounds having modulatory activity towards GCN2 are needed as therapeutic agents for treating cancer, with additional applications in the treatment of neurodegenerative diseases and doxorubicin-induced cardiotoxicity.

### SUMMARY OF THE INVENTION

Provided herein are compounds and compositions for the modulation of GCN2 (e.g., the activation or inhibition of GCN2). In various embodiments, the compounds and compositions described herein are useful for the treatment of GCN2 mediated conditions, diseases, or disorders (e.g., cancers and neurodegenerative diseases).

In one aspect, provided herein is a compound represented by Formula (Ia):

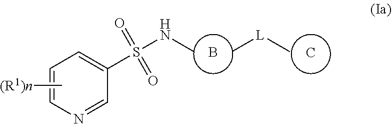

or a pharmaceutically acceptable salt thereof, wherein:
B is phenylene or 5-6 membered heteroarylene, wherein the phenylene or the 5-6 membered heteroarylene may be optionally substituted on one or more available carbons by one, two, three, or more substituents each independently selected from R²;
C is 5-10 membered heteroaryl or 7-10 membered heterocyclyl, wherein the 5-10 membered heteroaryl or 7-10 membered heterocycyl may be optionally substituted on one or more available carbons by one, two, three, or more substituents each independently selected from R³; and wherein, if the 5-10 membered heteroaryl or 7-10 membered heterocyclyl contains a substitutable ring nitrogen atom, that ring nitrogen atom may be optionally substituted by C₁₋₆alkyl;
L is selected from the group consisting of C₁₋₆alkylene, C₁₋₆alkylene-O—, and C₁₋₆ alkylene-N(R^{A})—;
R¹ is independently, for each occurrence, selected from the group consisting of halogen, C₁₋₆alkyl, C₃₋₆cycloalkyl, cyano, hydroxyl, C₁₋₆alkoxyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from halogen and hydroxyl;
R² is independently, for each occurrence, selected from the group consisting of halogen, C₁₋₆alkyl, cyano, C₁₋₆alkoxyl, and C₃₋₆cycloalkyl, wherein the C₁₋₆alkyl and C₃₋₆cycloalkyl may be optionally substituted on one or more available carbons by one, two, three, or more independent halogen substituents;
R³ is independently, for each occurrence, selected from the group consisting of halogen, C₁₋₆alkyl, C₂₋₆alkynyl, C₃₋₆cycloalkyl, cyano, C₁₋₆alkoxyl, hydroxyl, oxo, phenyl, —C(O)N(R^{A})(R^{B}), —N(R^{A})(R^{B}), 5-6 membered heterocyclyl, and 5-6 membered heteroaryl, wherein the C₁₋₆alkyl may be optionally substituted on one or more available carbons by one, two, three, or more substituents each independently selected from halogen and phenyl; and wherein if the 5-6 membered heterocyclyl contains a substitutable ring nitrogen atom, that ring nitrogen atom may be optionally substituted by C₁₋₆alkyl; or
two R³ substituents together with the carbon to which they are attached form C₃₋₆cycloalkyl;
R^{A} is independently, for each occurrence, hydrogen or C₁₋₆alkyl;
R^{B} is independently, for each occurrence, hydrogen or C₁₋₆alkyl; and
n=0, 1, 2, 3, or 4;
wherein the compound of Formula (Ia) is not

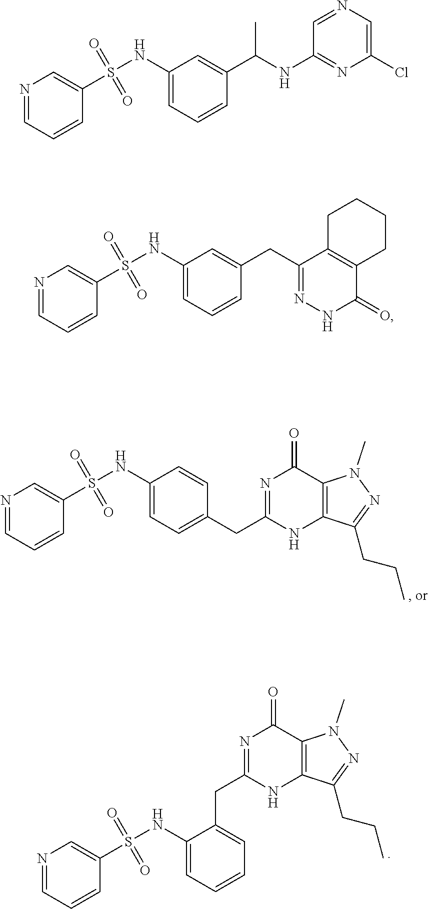 [SMILES: CC(Nc1cncc(Cl)n1)c1cccc(NS(=O)(=O)c2cccnc2)c1 ; O=c1[nH]nc(Cc2cccc(NS(=O)(=O)c3cccnc3)c2)c2c1CCCC2 ; CCCc1nn(C)c2c(=O)nc(Cc3ccc(NS(=O)(=O)c4cccnc4)cc3)[nH]c12 ; CCCc1nn(C)c2c(=O)nc(Cc3ccccc3NS(=O)(=O)c3cccnc3)[nH]c12]

In another aspect, provided herein is a compound represented by Formula (Ia):

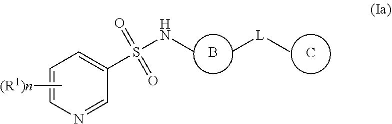

or a pharmaceutically acceptable salt thereof, wherein:
B is phenylene or 5-6 membered heteroarylene, wherein the phenylene or the 5-6 membered heteroarylene may be optionally substituted on one or more available carbons by one, two, three, or more substituents each independently selected from R²;
C is 5-10 membered heteroaryl or 7-10 membered heterocyclyl, wherein the 5-10 membered heteroaryl or 7-10 membered heterocyclyl may be optionally substituted on one or more available carbons by one, two, three, or more substituents each independently selected from R³; and wherein, if the 5-10 membered heteroaryl or 7-10 membered heterocyclyl contains a substitutable ring nitrogen atom, that ring nitrogen atom may be optionally substituted by C₁₋₆alkyl;
L is selected from the group consisting of C₁₋₆alkylene, C₁₋₆alkylene-O—, and C₁₋₆ alkylene-N(R^{A})—;
R¹ is independently, for each occurrence, selected from the group consisting of halogen, C₁₋₆alkyl, C₃₋₆cycloalkyl, cyano, hydroxyl, C₁₋₆alkoxyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from halogen and hydroxyl;
R² is independently, for each occurrence, selected from the group consisting of halogen, C₁₋₆alkyl, cyano, C₁₋₆alkoxyl, and C₃₋₆cycloalkyl, wherein the C₁₋₆alkyl and C₃₋₆cycloalkyl may be optionally substituted on one or more available carbons by one, two, three, or more independent halogen substituents;
R³ is independently, for each occurrence, selected from the group consisting of halogen, C₁₋₆alkyl, C₂₋₆alkynyl, C₃₋₆cycloalkyl, cyano, C₁₋₆alkoxyl, hydroxyl, oxo, phenyl, —C(O)N(R^{A})(R^{B}), —N(R^{A})(R^{B}), 5-6 membered heterocyclyl, and 5-6 membered heteroaryl, wherein the C₁₋₆alkyl may be optionally substituted on one or more available carbons by one, two, three, or more substituents each independently selected from halogen and phenyl; and wherein if the 5-6 membered heterocyclyl contains a substitutable ring nitrogen atom, that ring nitrogen atom may be optionally substituted by C₁₋₆alkyl; or
two R³ substituents together with the carbon to which they are attached form C₃₋₆cycloalkyl;
R^{A} is independently, for each occurrence, hydrogen or C₁₋₆alkyl;
R^{B} is independently, for each occurrence, hydrogen or C₁₋₆alkyl; and
n=1, 2, 3, or 4.

In another aspect, provided herein is a compound represented by Formula (Ib):

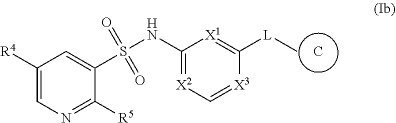

or a pharmaceutically acceptable salt thereof, wherein:
C is 5-10 membered heteroaryl or 7-10 membered heterocyclyl, wherein the 5-10 membered heteroaryl or 7-10 membered heterocyclyl may be optionally substituted on one or more available carbons by one, two, three, or more substituents each independently selected from R³; and wherein, if the 5-10 membered heteroaryl or 7-10 membered heterocyclyl contains a substitutable ring nitrogen atom, that ring nitrogen atom may be optionally substituted by C₁₋₆alkyl;
L is selected from the group consisting of C₁₋₆alkylene, C₁₋₆alkylene-O—, and C₁₋₆ alkylene-N(R^{A})—;
R³ is independently, for each occurrence, selected from the group consisting of C₁₋₆alkyl, C₂₋₆alkynyl, C₃₋₆cycloalkyl, cyano, C₁₋₆alkoxyl, oxo, phenyl, —C(O)N(R^{A})(R^{B}), —N(R^{A})(R^{B}), 6 membered heterocyclyl, and 5-6 membered heteroaryl, wherein the C₁₋₆alkyl may each individually be optionally substituted on one or more available carbons by one, two, three, or more substituents each independently selected from halogen and phenyl; and wherein if the 5-6 membered heterocyclyl contains a substitutable ring nitrogen atom, that ring nitrogen atom may be optionally substituted by C₁₋₆alkyl; or
two R³ substituents together with the carbon to which they are attached form C₃₋₆cycloalkyl;
X¹ is selected from CR⁶ and N;
X² is selected from CH and N;
X³ is selected from CR⁷ and N;
R⁴ is selected from halogen and cyano;
R⁵ is selected from C₁₋₆alkyl and C₁₋₆alkoxyl;
R⁶ is selected from halogen and cyano;
R⁷ is halogen; and
R^{A} and R^{B} are independently, for each occurrence, hydrogen or C₁₋₆alkyl.

In some embodiments, a compound provided herein is selected from a compound set forth in Table 1, or a pharmaceutically acceptable salt thereof.

In another aspect, provided herein is a pharmaceutical composition comprising a compound described herein and a pharmaceutically acceptable carrier.

In another aspect, provided herein are methods of treating cancer in a subject in need thereof, the method generally comprises administering to the subject a therapeutically effective amount of a compound described herein. In some embodiments, the cancer is selected from the group consisting of colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, sweat gland carcinoma, sebaceous gland carcinoma, thyroid cancer, kidney cancer, uterus cancer, esophagus cancer, liver cancer, head cancer, neck cancer, throat cancer, mouth cancer, bone cancer, chest cancer, lymph node cancer, eye cancer, mesothelioma, an acoustic neuroma, oligodendroglioma, meningioma, neuroblastoma, retinoblastoma, leukemia, lymphoma, and a combination thereof. In some embodiments, the cancer is selected from the group consisting of colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, leukemia, lymphoma, and a combination thereof.

In another aspect, provided herein are methods of treating a neurodegenerative disease in a subject in need thereof, the method generally comprises administering to the subject a therapeutically effective amount of a compound described herein. In some embodiments, the neurodegenerative disease is selected from the group consisting of Alzheimer's disease, Parkinson's Disease, Huntington's Disease, amyotrophic lateral sclerosis, and spinocerebellar ataxia.

In another aspect, provided herein are methods of modulating (both activation and/or inhibition) the activity of GCN2, the method generally comprises exposing GCN2 to an effective amount of a compound described herein to modulate the activity of said GCN2.

### DETAILED DESCRIPTION OF THE INVENTION

The invention provides GCN2-interacting compounds and related compounds, pharmaceutical compositions, and their use in the treatment of medical conditions, such as cancer, neurodegenerative diseases, and doxorubicin-induced cardiotoxicity, and in modulating (inhibiting/activating) GCN2 activity. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “—O-alkyl” etc.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. Exemplary alkylene groups include —CH₂—, —CH₂CH₂—, and —CH₂C(H)(CH₃)CH₂—. The term “—(C₀ alkylene)-” refers to a bond. Accordingly, the term “—(C₀₋₃ alkylene)-” encompasses a bond (i.e., C₀) and a —(C₁₋₃ alkylene) group.

As used herein, “carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In certain embodiments, a carbocyclyl group has 3 to 8 ring carbon atoms. In certain embodiments, a carbocyclyl group has 3 to 7 ring carbon atoms (“C3-7 carbocycyl”). In certain embodiments, a carbocyclyl group has 3 to 6 ring carbon atoms (“C3-6 carbocyclyl”). In certain embodiments, a carbocyclyl group has 5 to 10 ring carbon atoms (“C5-10 carbocyclyl”). In certain embodiments, a carbocyclyl group has 5 to 10 ring carbon atoms (“C7-10 carbocyclyl”). Exemplary C3-6 carbocyclyl groups include, without limitation, cyclopropyl (C3), cyclobutyl (C4), cyclobutenyl (C4), cyclopentyl (C5), cyclopentenyl (C5), cyclohexyl (C6), cyclohexenyl (C6), cyclohexadienyl (C6), and the like. Exemplary C3-8 carbocyclyl groups include, without limitation, the aforementioned C3-6 carbocyclyl groups as well as cycloheptyl (C7), cycloheptenyl (C7), cycloheptadienyl (C7), cycloheptatrienyl (C7), cyclooctyl (C8), cyclooctenyl (C8), bicyclo[2.2.1]heptanyl (C7), bicyclo[2.2.2]octanyl (C8), and the like. Exemplary C3-10 carbocyclyl groups include, without limitation, the aforementioned C3-8 carbocyclyl groups as well as cyclononyl (C9), cyclononenyl (C9), cyclodecyl (C10), cyclodecenyl (C10), octahydro-1H-indenyl (C9), decahydronaphthalenyl (C10), spiro[4.5]decanyl (C10), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or partially unsaturated.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “halocycloalkyl” refers to a cycloalkyl group that is substituted with at least one halogen.

The term “cycloalkylene” refers to a diradical of a cycloalkyl group. Exemplary cycloalkylene groups include

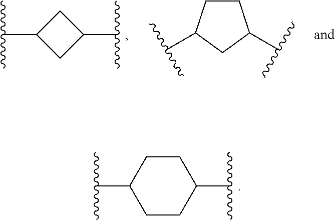

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The term “hydroxyfluoroalkyl” refers to a hydroxyalkyl that is substituted with at least one fluoro.

The term “aralkyl” refers to an alkyl group substituted with an aryl group. Exemplary aralkyl groups include

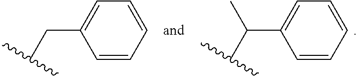

The term “heteroaralkyl” refers to an alkyl group substituted with a heteroaryl group.

The terms “alkenyl” and “alkynyl” are art-recognized and refer to unsaturated aliphatic groups analogous in length and possible substitution to the alkyls described above, but that contain at least one double or triple bond respectively.

The term “cycloalkenyl” refers to a monovalent unsaturated cyclic, bicyclic, or bridged (e.g., adamantyl) carbocyclic hydrocarbon containing at least one C—C double bond. In certain embodiments, the cycloalkenyl contains 5-10, 5-8, or 5-6 carbons, referred to herein, e.g., as “C₅-C₆ cycloalkenyl”. Exemplary cycloalkenyl groups include cyclohexenyl and cyclopentenyl.

The term “aryl” is art-recognized and refers to a carbocyclic aromatic group. Representative aryl groups include phenyl, naphthyl, anthracenyl, and the like. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “aryl” also includes polycyclic aromatic ring systems having two or more carbocyclic rings in which two or more carbons are common to two adjoining rings (the rings are “fused rings”) wherein all of the fused rings are aromatic rings, e.g., in a naphthyl group.

The term “phenylene” refers to a diradical of a phenyl group. Exemplary phenylene groups include

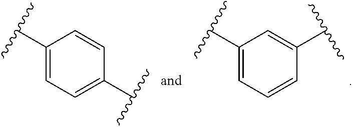

The term “heteroaryl” refers to a radical of a 5-10 membered monocyclic or bicyclic 4n+2 aromatic ring system (e.g., having 6 or 10 n electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

The term “heteroarylene” refers to a diradical of a heteroaryl group. Exemplary heteroarylene groups include: phenylene, pyridinylene, pyridazinylene, pyrimidinylene, pyrazinylene,

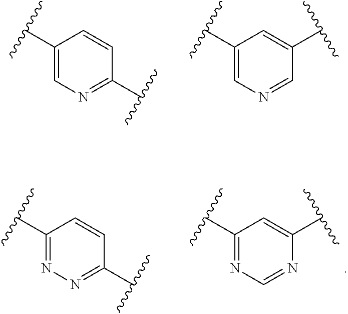

The terms ortho, meta, and para are art-recognized and refer to 1,2-, 1,3- and 1,4-disubstituted benzenes, respectively. For example, the names 1,2-dimethylbenzene and ortho-dimethylbenzene are synonymous.

The term “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C6 aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

The term “heterocycloalkyl” refers to a saturated heterocyclyl group having, for example, 3-7 ring atoms selected from carbon and heteroatoms (e.g., O, N, or S).

The terms “amine” and “amino” are art-recognized and refer to both unsubstituted and substituted amines, e.g., a moiety that may be represented by the general formulas:

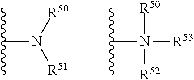

wherein R⁵⁰, R⁵¹, R⁵² and R⁵³ each independently represent a hydrogen, an alkyl, an alkenyl, —(CH₂)ₘ-R⁶¹, or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In certain embodiments, only one of R⁵⁰ or R⁵¹ may be a carbonyl, e.g., R⁵⁰, R⁵¹ and the nitrogen together do not form an imide. In other embodiments, R⁵⁰ and R⁵¹ (and optionally R⁵²) each independently represent a hydrogen, an alkyl, an alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and —O—(CH₂)ₘ—R⁶¹, where m and R⁶¹ are described above.

The term “fluoroalkoxyl” refers to an alkoxyl group that is substituted with at least one fluoro group. Exemplary fluoroalkoxyl groups include —OCH₂F, —OCHF₂, —OCF₃, —OCH₂CF₃, —OCF₂CF₃, and the like.

The term “oxo” is art-recognized and refers to a “═O” substituent. For example, a cyclopentane substituted with an oxo group is cyclopentanone.

The symbols “ [symbol] ”, “*”, and “**” indicate a point of attachment.

The term “substituted” means that one or more hydrogens on the atoms of the designated group are replaced with a selection from the indicated group, provided that the atoms' normal valences under the existing circumstances are not exceeded, and that the substitution results in a stable compound. Combinations of substituents and/or variables are permissible only if such combinations result in stable compounds. The terms “stable compound” or “stable structure” refer to a compound that is sufficiently robust to survive isolation to a useful degree of purity from a reaction mixture, and formulation into an efficacious therapeutic agent.

When any substituent or variable occurs more than one time in any constituent or the compound of the invention, its definition on each occurrence is independent of its definition at every other occurrence, unless otherwise indicated.

It should also be noted that any carbon as well as heteroatom with unsatisfied valences in the text, schemes, examples and tables herein is assumed to have the sufficient number of hydrogen atom(s) to satisfy the valences.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

Certain compounds contained in compositions of the present invention may exist in particular geometric or stereoisomeric forms. Further, certain compounds described herein may be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. The compounds may contain one or more stereogenic centers. For example, asymmetric carbon atoms may be present in a substituent such as an alkyl group. All such isomers, as well as mixtures thereof, are intended to be included in this invention, such as, for example, racemic mixtures, single enantiomers, diastereomeric mixtures and individual diastereomers. Additional asymmetric centers may be present depending upon the nature of the various substituents on the molecule. Each such asymmetric center will independently produce two optical isomers, and it is intended that all of the possible optical isomers, diastereomers in mixtures, and pure or partially purified compounds are included within the ambit of this invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as an atropisomer (e.g., substituted biaryls), all forms of such atropisomer are considered part of this invention.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate (also known as toluenesulfonate), undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. Further examples of salts include, but are not limited to, ascorbate, borate, nitrate, phosphate, salicylate, and sulfate. Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66 (1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Additional exemplary basic salts include, but are not limited to: ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

The present invention includes the compounds of the invention in all their isolated forms (such as any solvates, hydrates, stereoisomers, and tautomers thereof). Further, the invention includes compounds in which one or more of the atoms may be artificially enriched in a particular isotope having the same atomic number, but an atomic mass or mass number different from the atomic mass or mass number predominantly found in nature. The present invention is meant to include all suitable isotopic variations of the compounds of the invention. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds can be prepared without undue experimentation by conventional techniques known to those skilled in the art or by processes analogous to those described in the Schemes and Examples herein using appropriate isotopically-enriched reagents and/or intermediates.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

The terms “a” and “an” as used herein mean “one or more” and include the plural unless the context is inappropriate.

As a general matter, compositions specifying a percentage are by weight unless otherwise specified.

### Compounds

In one aspect, provided herein are compounds represented by Formula (I):

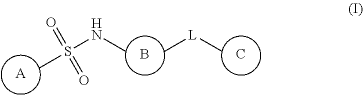

or a pharmaceutically acceptable salt thereof, wherein:
A is phenyl or 5-6 membered heteroaryl, wherein the phenyl or the 5-6 membered heteroaryl may be optionally substituted on one or more available carbons by one, two, three, or more substituents each independently selected from R¹;
B is phenylene or 5-6 membered heteroarylene, wherein the phenylene or the 5-6 membered heteroarylene may be optionally substituted on one or more available carbons by one, two, three, or more substituents each independently selected from R²;
C is 5-10 membered heteroaryl or 7-10 membered heterocyclyl, wherein the 5-10 membered heteroaryl or 7-10 membered heterocyclyl may be optionally substituted on one or more available carbons by one, two, three, or more substituents each independently selected from R³; and wherein, if the 5-10 membered heteroaryl or 7-10 membered heterocyclyl contains a substitutable ring nitrogen atom, that ring nitrogen atom may be optionally substituted by C₁₋₆alkyl;
L is selected from the group consisting of C₁₋₆alkylene, C₁₋₆alkylene-O—, and C₁₋₆ alkylene-N(R^{A})—;
R¹ is independently, for each occurrence, selected from the group consisting of halogen, C₁₋₆alkyl, C₃₋₆cycloalkyl, cyano, hydroxyl, C₁₋₆alkoxyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from halogen and hydroxyl;
R² is independently, for each occurrence, selected from the group consisting of halogen, C₁₋₆alkyl, cyano, C₁₋₆alkoxyl, and C₃₋₆cycloalkyl, wherein the C₁₋₆alkyl and C₃₋₆cycloalkyl may be optionally substituted on one or more available carbons by one, two, three, or more independent halogen substituents;
R³ is independently, for each occurrence, selected from the group consisting of halogen, C₁₋₆alkyl, C₂₋₆alkynyl, C₃₋₆cycloalkyl, cyano, C₁₋₆alkoxyl, hydroxyl, oxo, phenyl, —C(O)N(R^{A})(R^{B}), —N(R^{A})(R^{B}), 5-6 membered heterocyclyl, and 5-6 membered heteroaryl, wherein the C₁₋₆alkyl may be optionally substituted on one or more available carbons by one, two, three, or more substituents each independently selected from halogen and phenyl; and wherein if the 5-6 membered heterocyclyl contains a substitutable ring nitrogen atom, that ring nitrogen atom may be optionally substituted by C₁₋₆alkyl; or
two R³ substituents together with the carbon to which they are attached form C₃₋₆cycloalkyl;
R^{A} is independently, for each occurrence, hydrogen or C₁₋₆alkyl; and
R^{B} is independently, for each occurrence, selected from the group consisting of hydrogen, C₁₋₆alkyl, and C₃₋₆cycloalkyl;
wherein the compound of Formula (I) is not

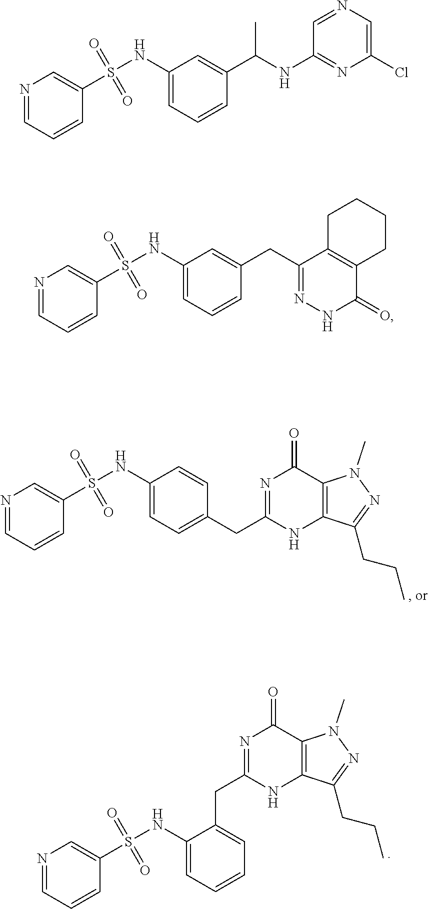 [SMILES: CC(Nc1cncc(Cl)n1)c1cccc(NS(=O)(=O)c2cccnc2)c1 ; O=c1[nH]nc(Cc2cccc(NS(=O)(=O)c3cccnc3)c2)c2c1CCCC2 ; CCCc1nn(C)c2c(=O)nc(Cc3ccc(NS(=O)(=O)c4cccnc4)cc3)[nH]c12 ; CCCc1nn(C)c2c(=O)nc(Cc3ccccc3NS(=O)(=O)c3cccnc3)[nH]c12]

In another aspect, provided herein are compounds represented by Formula (I):

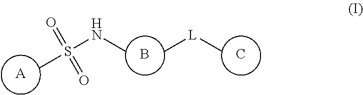

or a pharmaceutically acceptable salt thereof, wherein:
A is phenyl or 5-6 membered heteroaryl, wherein the phenyl or the 5-6 membered heteroaryl is substituted on one or more available carbons by one, two, three, or more substituents each independently selected from R¹;
B is phenylene or 5-6 membered heteroarylene, wherein the phenyl or the 5-6 membered heteroarylene is substituted on one or more available carbons by one, two, three, or more substituents each independently selected from R²;
C is 5-10 membered heteroaryl or 7-10 membered heterocyclyl, wherein the 5-10 membered heteroaryl or 7-10 membered heterocyclyl may be optionally substituted on one or more available carbons by one, two, three, or more substituents each independently selected from R³; and wherein, if the 5-10 membered heteroaryl or 7-10 membered heterocyclyl contains a substitutable ring nitrogen atom, that ring nitrogen atom may be optionally substituted by C₁₋₆alkyl;
L is selected from the group consisting of C₁₋₆alkylene, C₁₋₆alkylene-O—, and C₁₋₆ alkylene-N(R^{A})—;
R¹ is independently, for each occurrence, selected from the group consisting of halogen, C₁₋₆alkyl, cyano, and C₁₋₆alkoxyl, wherein the C₁₋₆alkyl may optionally be optionally substituted on one or more available carbons by one, two, three, or more independent halogen substituents;
R² is independently, for each occurrence, halogen or cyano;
R³ is independently, for each occurrence, selected from the group consisting of C₁₋₆alkyl, C₂₋₆alkynyl, C₃₋₆cycloalkyl, cyano, C₁₋₆alkoxyl, oxo, phenyl, —C(O)N(R^{A})(R^{B}), —N(R^{A})(R^{B}), 6 membered heterocyclyl, and 5-6 membered heteroaryl, wherein the C₁₋₆alkyl may each individually be optionally substituted on one or more available carbons by one, two, three, or more substituents each independently selected from halogen and phenyl; and wherein if the 5-6 membered heterocyclyl contains a substitutable ring nitrogen atom, that ring nitrogen atom may be optionally substituted by C₁₋₆alkyl; or
two R³ substituents together with the carbon to which they are attached form C₃₋₆cycloalkyl; and
R^{A} and R^{B} are independently, for each occurrence, hydrogen or C₁₋₆alkyl.

In some embodiments, A is phenyl or pyridinyl, wherein A is substituted with one, two, or three independent R¹ substituents selected from the group consisting of halogen, C₁₋₆alkyl, cyano, and C₁₋₆alkoxyl, wherein the C₁₋₆alkyl may be optionally substituted on one or more available carbons by one, two, three, or more independent halogen substituents.

In some embodiments, A is phenyl or pyridinyl, wherein A is substituted with two R¹ substituents selected from the group consisting of halogen, C₁₋₆alkyl, cyano, and C₁₋₆alkoxyl, wherein the C₁₋₆alkyl may be optionally substituted on one or more available carbons by one, two, three, or more independent halogen substituents. In some embodiments, A is phenyl or

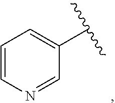

wherein A is substituted with two independent R₁ substituents selected from the group consisting of halogen, C₁₋₆alkyl, cyano, and C₁₋₆alkoxyl, wherein the C₁₋₆alkyl may be optionally substituted on one or more available carbons by one, two, three, or more independent halogen substituents.

In some embodiments, R₁ is independently, for each occurrence, selected from the group consisting of chloro, fluoro, cyano, CH₃, CF₃, and —O—CH₃.

In some embodiments, A is selected from the group consisting of

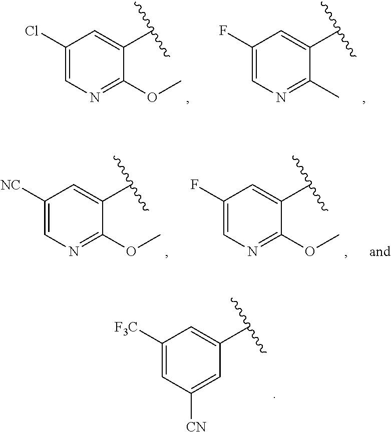

In another aspect, provided herein are compounds represented by Formula (I):

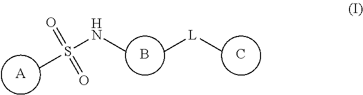

or a pharmaceutically acceptable salt thereof, wherein:
A is pyridinyl, wherein the pyridinyl may be optionally substituted on one or more available carbons by one, two, three, or more substituents each independently selected from R¹;
B is phenylene or 5-6 membered heteroarylene, wherein the phenylene or the 5-6 membered heteroarylene may be optionally substituted on one or more available carbons by one, two, three, or more substituents each independently selected from R²;
C is 5-10 membered heteroaryl or 7-10 membered heterocyclyl, wherein the 5-10 membered heteroaryl or 7-10 membered heterocyclyl may be optionally substituted on one or more available carbons by one, two, three, or more substituents each independently selected from R³; and wherein, if the 5-10 membered heteroaryl or 7-10 membered heterocyclyl contains a substitutable ring nitrogen atom, that ring nitrogen atom may be optionally substituted by C₁₋₆alkyl;
L is selected from the group consisting of C₁₋₆alkylene, C₁₋₆alkylene-O—, and C₁₋₆ alkylene-N(R^{A})—;
R¹ is independently, for each occurrence, selected from the group consisting of halogen, C₁₋₆alkyl, C₃₋₆cycloalkyl, cyano, hydroxyl, C₁₋₆alkoxyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from halogen and hydroxyl;
R² is independently, for each occurrence, selected from the group consisting of halogen, C₁₋₆alkyl, cyano, C₁₋₆alkoxyl, and C₃₋₆cycloalkyl, wherein the C₁₋₆alkyl and C₃₋₆cycloalkyl may be optionally substituted on one or more available carbons by one, two, three, or more independent halogen substituents;
R³ is independently, for each occurrence, selected from the group consisting of halogen, C₁₋₆alkyl, C₂₋₆alkynyl, C₃₋₆cycloalkyl, cyano, C₁₋₆alkoxyl, hydroxyl, oxo, phenyl, —C(O)N(R^{A})(R^{B}), —N(R^{A})(R^{B}), 5-6 membered heterocyclyl, and 5-6 membered heteroaryl, wherein the C₁₋₆alkyl may be optionally substituted on one or more available carbons by one, two, three, or more substituents each independently selected from halogen and phenyl; and wherein if the 5-6 membered heterocyclyl contains a substitutable ring nitrogen atom, that ring nitrogen atom may be optionally substituted by C₁₋₆alkyl; or
two R³ substituents together with the carbon to which they are attached form C₃₋₆cycloalkyl;
R^{A} is independently, for each occurrence, hydrogen or C₁₋₆alkyl; and
R^{B} is independently, for each occurrence, selected from the group consisting of hydrogen, C₁₋₆alkyl, and C₃₋₆cycloalkyl;
wherein the compound of Formula (I) is not

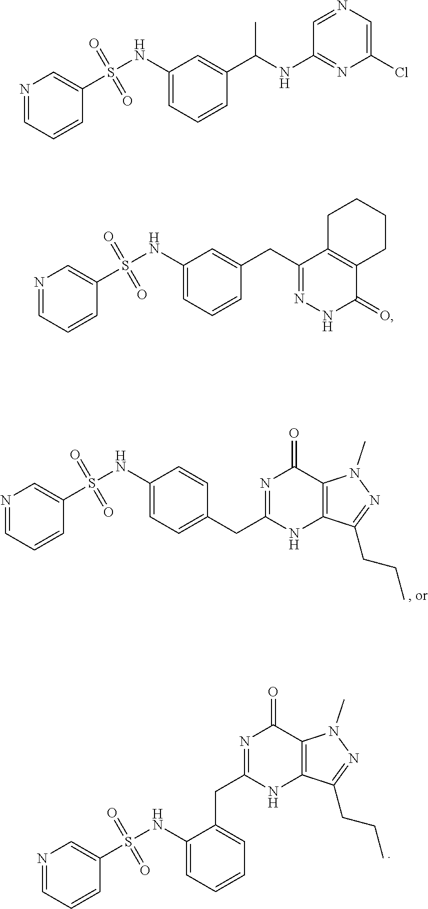 [SMILES: CC(Nc1cncc(Cl)n1)c1cccc(NS(=O)(=O)c2cccnc2)c1 ; O=c1[nH]nc(Cc2cccc(NS(=O)(=O)c3cccnc3)c2)c2c1CCCC2 ; CCCc1nn(C)c2c(=O)nc(Cc3ccc(NS(=O)(=O)c4cccnc4)cc3)[nH]c12 ; CCCc1nn(C)c2c(=O)nc(Cc3ccccc3NS(=O)(=O)c3cccnc3)[nH]c12]

In another aspect, provided herein are compounds represented by Formula (I):

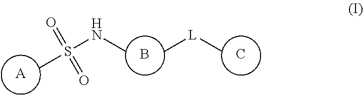

or a pharmaceutically acceptable salt thereof, wherein:
A is pyridinyl, wherein the pyridinyl is substituted on one or more available carbons by one, two, three, or more substituents each independently selected from R¹;
B is phenylene or 5-6 membered heteroarylene, wherein the phenyl or the 5-6 membered heteroarylene is substituted on one or more available carbons by one, two, three, or more substituents each independently selected from R²;
C is 5-10 membered heteroaryl or 7-10 membered heterocyclyl, wherein the 5-10 membered heteroaryl or 7-10 membered heterocyclyl may be optionally substituted on one or more available carbons by one, two, three, or more substituents each independently selected from R³; and wherein, if the 5-10 membered heteroaryl or 7-10 membered heterocyclyl contains a substitutable ring nitrogen atom, that ring nitrogen atom may be optionally substituted by C₁₋₆alkyl;
L is selected from the group consisting of C₁₋₆alkylene, C₁₋₆alkylene-O—, and C₁₋₆ alkylene-N(R^{A})—;
R¹ is independently, for each occurrence, selected from the group consisting of halogen, C₁₋₆alkyl, cyano, and C₁₋₆alkoxyl, wherein the C₁₋₆alkyl may optionally be optionally substituted on one or more available carbons by one, two, three, or more independent halogen substituents;
R² is independently, for each occurrence, halogen or cyano;
R³ is independently, for each occurrence, selected from the group consisting of C₁₋₆alkyl, C₂₋₆alkynyl, C₃₋₆cycloalkyl, cyano, C₁₋₆alkoxyl, oxo, phenyl, —C(O)N(R^{A})(R^{B}), —N(R^{A})(R^{B}), 6 membered heterocyclyl, and 5-6 membered heteroaryl, wherein the C₁₋₆alkyl may each individually be optionally substituted on one or more available carbons by one, two, three, or more substituents each independently selected from halogen and phenyl; and wherein if the 5-6 membered heterocyclyl contains a substitutable ring nitrogen atom, that ring nitrogen atom may be optionally substituted by C₁₋₆alkyl; or
two R³ substituents together with the carbon to which they are attached form C₃₋₆cycloalkyl; and
R^{A} and R^{B} are independently, for each occurrence, hydrogen or C₁₋₆alkyl.

In some embodiments, A is pyridinyl, wherein the pyridinyl is substituted with two R¹ substituents selected from the group consisting of halogen, C₁₋₆alkyl, cyano, and C₁₋₆alkoxyl.

In some embodiments, A is

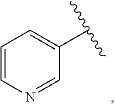

wherein A is substituted with two independent R₁ substituents selected from the group consisting of halogen, C₁₋₆alkyl, cyano, and C₁₋₆alkoxyl.

In some embodiments, R₁ is independently, for each occurrence, selected from the group consisting of chloro, fluoro, cyano, CH₃, and —O—CH₃.

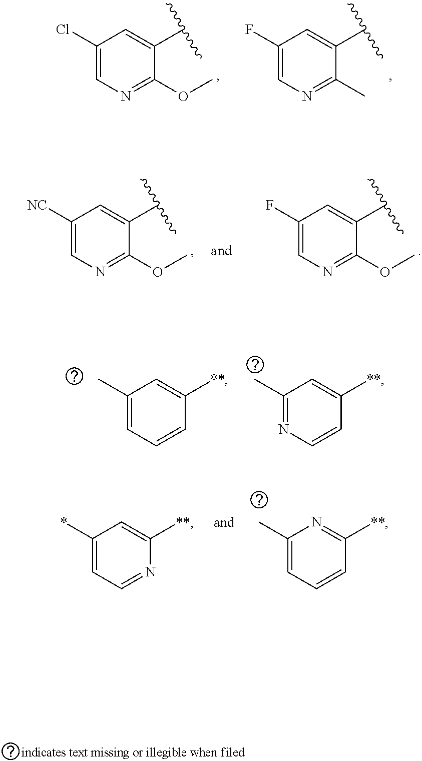

wherein * denotes the point of attachment to

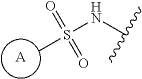

and * * denotes the point of attachment to

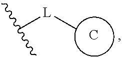

wherein B may be optionally substituted with one or two independent R² substituents selected from halogen or cyano. In some embodiments, B is

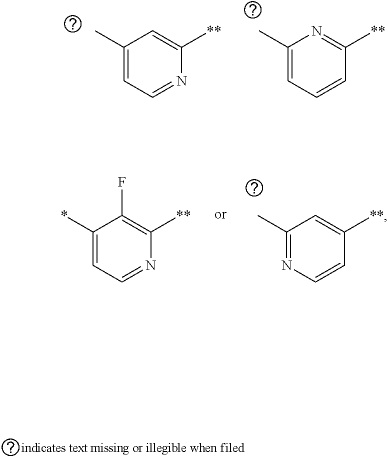

In some embodiments, B is

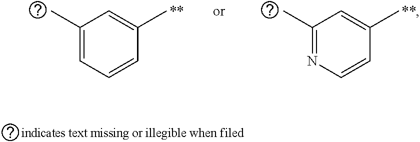

wherein B is substituted with two independent R² substituents selected from halogen or cyano. In some embodiments, R² is independently, for each occurrence, selected from the group consisting of chloro, fluoro, and cyano. In some embodiments, B is selected from the group consisting of

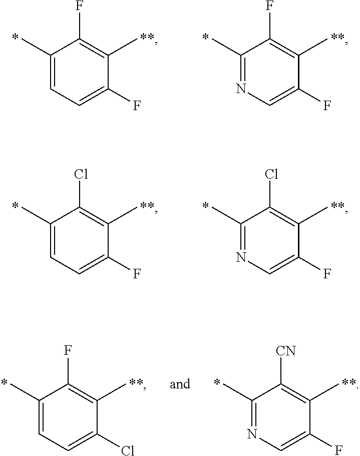

In some embodiments, L is selected from the group consisting of —CH₂—CH₂—, —C(CH₃)(H)—O—, —CH₂—O—, —CH₂—N(H)—, and —C(CH₃)(H)—N(H)—. In some embodiments, L is selected from the group consisting of —CH₂—CH₂—, ^{#}—C(CH₃)(H)—O-^{##}, ^{#}—CH₂—O-^{##}, ^{#}—O—CH₂-^{##}, ^{#}—CH₂—N(H)-^{##}, ^{#}—N(H)—CH₂-^{##}, and ^{#}—C(CH₃)(H)—N(H)-^{##}, wherein ^{#} denotes the point of attachment to

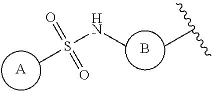

and ^{##} denotes the point of attachment to

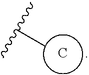

In another aspect, provided herein are compounds represented by Formula (Ia):

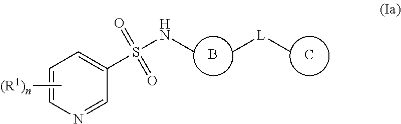

or a pharmaceutically acceptable salt thereof, wherein:
B is phenylene or 5-6 membered heteroarylene, wherein the phenylene or the 5-6 membered heteroarylene may be optionally substituted on one or more available carbons by one, two, three, or more substituents each independently selected from R²;
C is 5-10 membered heteroaryl or 7-10 membered heterocyclyl, wherein the 5-10 membered heteroaryl or 7-10 membered heterocycyl may be optionally substituted on one or more available carbons by one, two, three, or more substituents each independently selected from R³; and wherein, if the 5-10 membered heteroaryl or 7-10 membered heterocyclyl contains a substitutable ring nitrogen atom, that ring nitrogen atom may be optionally substituted by C₁₋₆alkyl;
L is selected from the group consisting of C₁₋₆alkylene, C₁₋₆alkylene-O—, and C₁₋₆ alkylene-N(R^{A})—;
R¹ is independently, for each occurrence, selected from the group consisting of halogen, C₁₋₆alkyl, C₃₋₆cycloalkyl, cyano, hydroxyl, C₁₋₆alkoxyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from halogen and hydroxyl;
R² is independently, for each occurrence, selected from the group consisting of halogen, C₁₋₆alkyl, cyano, C₁₋₆alkoxyl, and C₃₋₆cycloalkyl, wherein the C₁₋₆alkyl and C₃₋₆cycloalkyl may be optionally substituted on one or more available carbons by one, two, three, or more independent halogen substituents;
R³ is independently, for each occurrence, selected from the group consisting of halogen, C₁₋₆alkyl, C₂₋₆alkynyl, C₃₋₆cycloalkyl, cyano, C₁₋₆alkoxyl, hydroxyl, oxo, phenyl, —C(O)N(R^{A})(R^{B}), —N(R^{A})(R^{B}), 5-6 membered heterocyclyl, and 5-6 membered heteroaryl, wherein the C₁₋₆alkyl may be optionally substituted on one or more available carbons by one, two, three, or more substituents each independently selected from halogen and phenyl; and wherein if the 5-6 membered heterocyclyl contains a substitutable ring nitrogen atom, that ring nitrogen atom may be optionally substituted by C₁₋₆alkyl; or
two R³ substituents together with the carbon to which they are attached form C₃₋₆cycloalkyl;
R^{A} is independently, for each occurrence, hydrogen or C₁₋₆alkyl;
R^{B} is independently, for each occurrence, hydrogen or C₁₋₆alkyl; and
n=0, 1, 2, 3, or 4;
   wherein the compound of Formula (Ia) is not

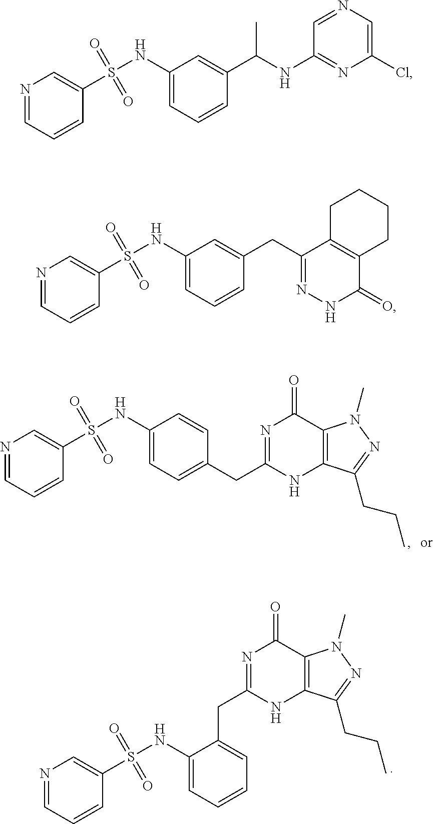 [SMILES: CC(Nc1cncc(Cl)n1)c1cccc(NS(=O)(=O)c2cccnc2)c1 ; O=c1[nH]nc(Cc2cccc(NS(=O)(=O)c3cccnc3)c2)c2c1CCCC2 ; CCCc1nn(C)c2c(=O)nc(Cc3ccc(NS(=O)(=O)c4cccnc4)cc3)[nH]c12 ; CCCc1nn(C)c2c(=O)nc(Cc3ccccc3NS(=O)(=O)c3cccnc3)[nH]c12]

In another aspect, provided herein are compounds represented by Formula (Ia)

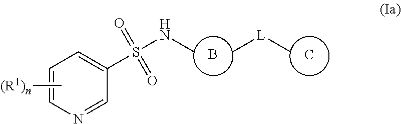

or a pharmaceutically acceptable salt thereof, wherein:
B is phenylene or 5-6 membered heteroarylene, wherein the phenylene or the 5-6 membered heteroarylene may be optionally substituted on one or more available carbons by one, two, three, or more substituents each independently selected from R²;
C is 5-10 membered heteroaryl or 7-10 membered heterocyclyl, wherein the 5-10 membered heteroaryl or 7-10 membered heterocyclyl may be optionally substituted on one or more available carbons by one, two, three, or more substituents each independently selected from R³; and wherein, if the 5-10 membered heteroaryl or 7-10 membered heterocyclyl contains a substitutable ring nitrogen atom, that ring nitrogen atom may be optionally substituted by C₁₋₆alkyl;
L is selected from the group consisting of C₁₋₆alkylene, C₁₋₆alkylene-O—, and C₁₋₆ alkylene-N(R^{A})—;
R¹ is independently, for each occurrence, selected from the group consisting of halogen, C₁₋₆alkyl, C₃₋₆cycloalkyl, cyano, hydroxyl, C₁₋₆alkoxyl, —N(R^{A})(R^{B}), —N(R^{A})—C(O)(R^{B}), —(C₁₋₆alkylene)-N(R^{A})(R^{B}), —CO₂H, —CO₂(C₁₋₆alkyl), and —S—(C₁₋₆alkyl), wherein the C₁₋₆alkyl, C₁₋₆alkoxyl, and —S—(C₁₋₆alkyl) may be optionally substituted on one or more available carbons by one, two, three, or more substituents each independently selected from halogen and hydroxyl;
R² is independently, for each occurrence, selected from the group consisting of halogen, C₁₋₆alkyl, cyano, C₁₋₆alkoxyl, and C₃₋₆cycloalkyl, wherein the C₁₋₆alkyl and C₃₋₆cycloalkyl may be optionally substituted on one or more available carbons by one, two, three, or more independent halogen substituents;
R³ is independently, for each occurrence, selected from the group consisting of halogen, C₁₋₆alkyl, C₂₋₆alkynyl, C₃₋₆cycloalkyl, cyano, C₁₋₆alkoxyl, hydroxyl, oxo, phenyl, —C(O)N(R^{A})(R^{B}), —N(R^{A})(R^{B}), 5-6 membered heterocyclyl, and 5-6 membered heteroaryl, wherein the C₁₋₆alkyl may be optionally substituted on one or more available carbons by one, two, three, or more substituents each independently selected from halogen and phenyl; and wherein if the 5-6 membered heterocyclyl contains a substitutable ring nitrogen atom, that ring nitrogen atom may be optionally substituted by C₁₋₆alkyl; or
two R³ substituents together with the carbon to which they are attached form C₃₋₆cycloalkyl;
R^{A} is independently, for each occurrence, hydrogen or C₁₋₆alkyl;
R^{B} is independently, for each occurrence, hydrogen or C₁₋₆alkyl; and
n=1, 2, 3, or 4.

In some embodiments, n is 2. In some embodiments, R₁ is independently, for each occurrence, selected from the group consisting of halogen, C₁₋₆alkyl, cyano, and C₁₋₆alkoxyl. In some embodiments, R₁ is independently, for each occurrence, selected from the group consisting of chloro, fluoro, cyano, CH₃, and —O—CH₃.

In some embodiments, B is phenylene or pyridinylene, wherein B may be optionally substituted with one, two, or three R² substituents independently selected from halogen or cyano. In some embodiments, B is selected from the group consisting of

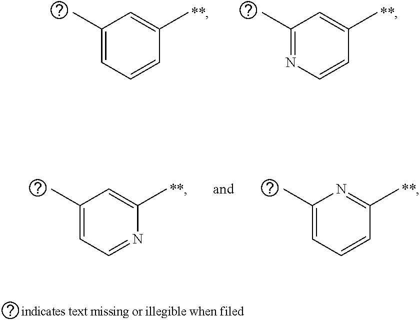

wherein * denotes the point of attachment to

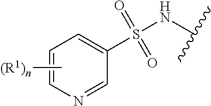

and * * denotes the point of attachment to

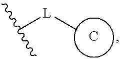

wherein B may be optionally substituted with one or two R² substituents independently selected from halogen or cyano. In some embodiments, B is

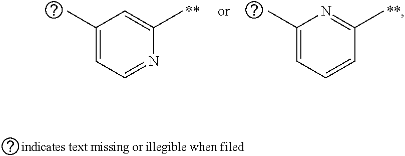

wherein B is substituted with one R² substituent selected from halogen or cyano. In some embodiments, R² is fluoro. In some embodiments, B is

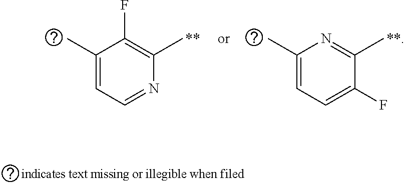

In some embodiments, The compound of claim 7, wherein B is

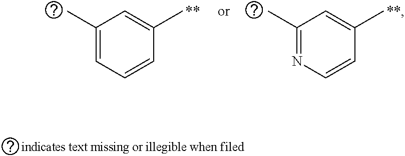

wherein B is substituted with two independent R² substituents selected from halogen or cyano. In some embodiments, R² is independently, for each occurrence, selected from the group consisting of chloro, fluoro, and cyano. In some embodiments, B is selected from the group consisting of

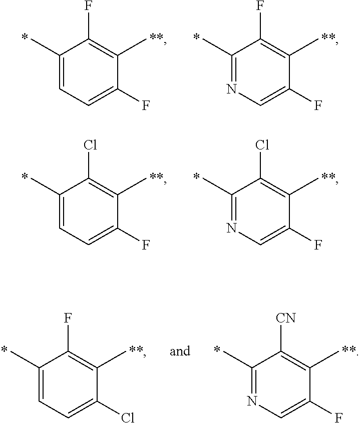

In some embodiments, L is selected from the group consisting of —CH₂—CH₂—, —C(CH₃)(H)—O—, —CH₂—O—, —CH₂—N(H)—, and —C(CH₃)(H)—N(H)—. In some embodiments, L is selected from the group consisting of —CH₂—CH₂—, ^{#}—C(CH₃)(H)—O—^{##}, ^{#}—CH₂—O-^{##}, ^{#}—O—CH₂ ^{##}, ^{#}—CH₂—N(H)-^{##}, ^{#}—N(H)—CH₂^{##}, and ^{#}—C(CH₃)(H)—N(H)-^{##}, wherein ^{#} denotes the point of attachment to

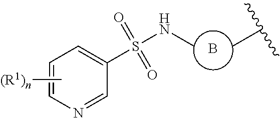

and ^{##} denotes the point of attachment to

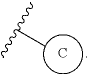

In another aspect, provided herein are compounds represented by Formula (Ib):

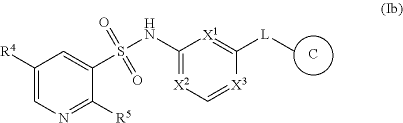

or a pharmaceutically acceptable salt thereof, wherein:
C is 5-10 membered heteroaryl or 7-10 membered heterocyclyl, wherein the 5-10 membered heteroaryl or 7-10 membered heterocyclyl may be optionally substituted on one or more available carbons by one, two, three, or more substituents each independently selected from R³; and wherein, if the 5-10 membered heteroaryl or 7-10 membered heterocyclyl contains a substitutable ring nitrogen atom, that ring nitrogen atom may be optionally substituted by C₁₋₆alkyl;
L is selected from the group consisting of C₁₋₆alkylene, C₁₋₆alkylene-O—, and C₁₋₆ alkylene-N(R^{A})—;
R³ is independently, for each occurrence, selected from the group consisting of C₁₋₆alkyl, C₂₋₆alkynyl, C₃₋₆cycloalkyl, cyano, C₁₋₆alkoxyl, oxo, phenyl, —C(O)N(R^{A})(R^{B}), —N(R^{A})(R^{B}), 6 membered heterocyclyl, and 5-6 membered heteroaryl, wherein the C₁₋₆alkyl may each individually be optionally substituted on one or more available carbons by one, two, three, or more substituents each independently selected from halogen and phenyl; and wherein if the 5-6 membered heterocyclyl contains a substitutable ring nitrogen atom, that ring nitrogen atom may be optionally substituted by C₁₋₆alkyl; or
two R³ substituents together with the carbon to which they are attached form C₃₋₆cycloalkyl;
X¹ is selected from CR⁶ and N;
X² is selected from CH and N;
X³ is selected from CR⁷ and N;
R⁴ is selected from halogen and cyano;
R⁵ is selected from C₁₋₆alkyl and C₁₋₆alkoxyl;
R⁶ is selected from halogen and cyano;
R⁷ is halogen; and
R^{A} and R^{B} are independently, for each occurrence, hydrogen or C₁₋₆alkyl.

In some embodiments, X¹ is CR⁶; X² is CH; and X³ is CR⁷. In some embodiments, R⁶ is selected from the group consisting of chloro, fluoro, and cyano. In some embodiments, R⁷ is chloro or fluoro.

In some embodiments, X¹ is CR⁶; X² is N; and X³ is CR⁷. In some embodiments, R⁶ is chloro or fluoro. In some embodiments, R⁷ is fluoro.

In some embodiments, X¹ is CR⁶; X² is CH; and X³ is N. In some embodiments, R⁶ is fluoro.

In some embodiments, X¹ is N; X² is CH; and X³ is CR⁷. In some embodiments, R⁷ is fluoro.

In some embodiments, L is selected from the group consisting of —CH₂—CH₂—, —C(CH₃)(H)—O—, —CH₂—O—, —CH₂—N(H)—, and —C(CH₃)(H)—N(H)—. In some embodiments, L is selected from the group consisting of —CH₂—CH₂—, ^{#}—C(CH₃)(H)—O^{##}, ^{#}—CH₂—O^{##}, ^{#}—O—CH₂-^{##}, ^{#}—CH₂—N(H)-^{##}, ^{#}—N(H)—CH₂-^{##}, and ^{#}—C(CH₃)(H)—N(H)—^{##}, wherein ^{#} denotes the point of attachment

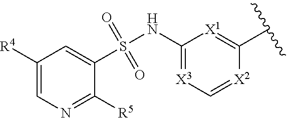

and ^{##} denotes the point of attachment to

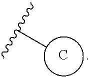

In another aspect, provided herein are compounds represented by Formula (Ic):

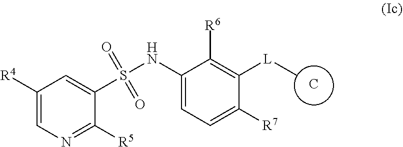

or a pharmaceutically acceptable salt thereof, wherein:
C is 5-10 membered heteroaryl or 7-10 membered heterocyclyl, wherein the 5-10 membered heteroaryl or 7-10 membered heterocyclyl may be optionally substituted on one or more available carbons by one, two, three, or more substituents each independently selected from R³; and wherein, if the 5-10 membered heteroaryl or 7-10 membered heterocyclyl contains a substitutable ring nitrogen atom, that ring nitrogen atom may be optionally substituted by C₁₋₆alkyl;
L is selected from the group consisting of C₁₋₆alkylene, C₁₋₆alkylene-O—, and C₁₋₆ alkylene-N(R^{A})—;
R³ is independently, for each occurrence, selected from the group consisting of C₁₋₆alkyl, C₂₋₆alkynyl, C₃₋₆cycloalkyl, cyano, C₁₋₆alkoxyl, oxo, phenyl, —C(O)N(R^{A})(R^{B}), and —N(R^{A})(R^{B}), wherein the C₁₋₆alkyl may be optionally substituted on one or more available carbons by one, two, three, or more independent halogen substituents; or
two R³ substituents together with the carbon to which they are attached form C₃₋₆cycloalkyl;
R⁴ is halogen or cyano;
R⁵ is C₁₋₆alkyl or C₁₋₆alkoxyl;
R⁶ is halogen or cyano;
R⁷ is halogen; and
R^{A} and R^{B} are independently, for each occurrence, hydrogen or C₁₋₆alkyl.

In some embodiments, R⁴ is selected from the group consisting of chloro, fluoro, and cyano.

In some embodiments, R⁵ is CH₃ or —O—CH₃.

In some embodiments, R⁶ is selected from the group consisting of chloro, fluoro, and cyano.

In some embodiments, R⁷ is chloro or fluoro.

In some embodiments, L is selected from the group consisting of —CH₂—CH₂—, —C(CH₃)(H)—O—, —CH₂—O—, —CH₂—N(H)—, and —C(CH₃)(H)—N(H)—. In some embodiments, L is selected from the group consisting of —CH₂—CH₂—, —C(CH₃)(H)—O—, —CH₂—O—, —CH₂—N(H)—, and —C(CH₃)(H)—N(H)—. In some embodiments, L is selected from the group consisting of —CH₂—CH₂—, ^{#}—C(CH₃)(H)—O—^{##}, ^{#}—CH₂—O^{##}, ^{#}—O—CH₂^{##}, ^{#}—CH₂—N(H)-^{##}, ^{#}—N(H)—CH₂-^{##}, and ^{#}—C(CH₃)(H)—N(H)-^{##}, wherein ^{#} denotes the point of attachment to

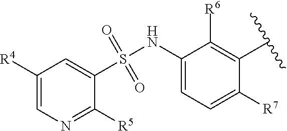

and ##denotes the point of attachment to

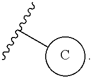

In some embodiments, C is selected from the group consisting of

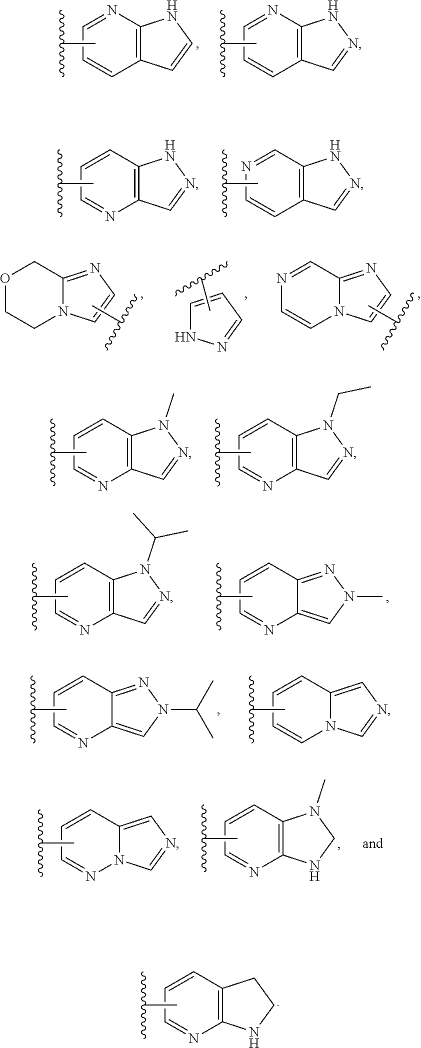

wherein C may be optionally substituted on one or more available carbons by one, two, three, or more R³ substituents independently selected from the group consisting of C₁₋₆alkyl, C₂-6alkynyl, C₃₋₆cycloalkyl, cyano, C₁₋₆alkoxyl, oxo, phenyl, —C(O)N(R^{A})(R^{B}), —N(R^{A})(R^{B}), 5-6 membered heterocyclyl, and 5-6 membered heteroaryl, wherein
the C₁₋₆alkyl may be optionally substituted on one or more available carbons by one, two, three, or more substituents each independently selected from halogen and phenyl;
if the 5-6 membered heterocyclyl contains a substitutable ring nitrogen atom, that ring nitrogen atom may be optionally substituted by C₁₋₆alkyl; and
R^{A} and R^{B} are independently, for each occurrence, hydrogen or C₁₋₆alkyl.

In some embodiments, C is selected from the group consisting of

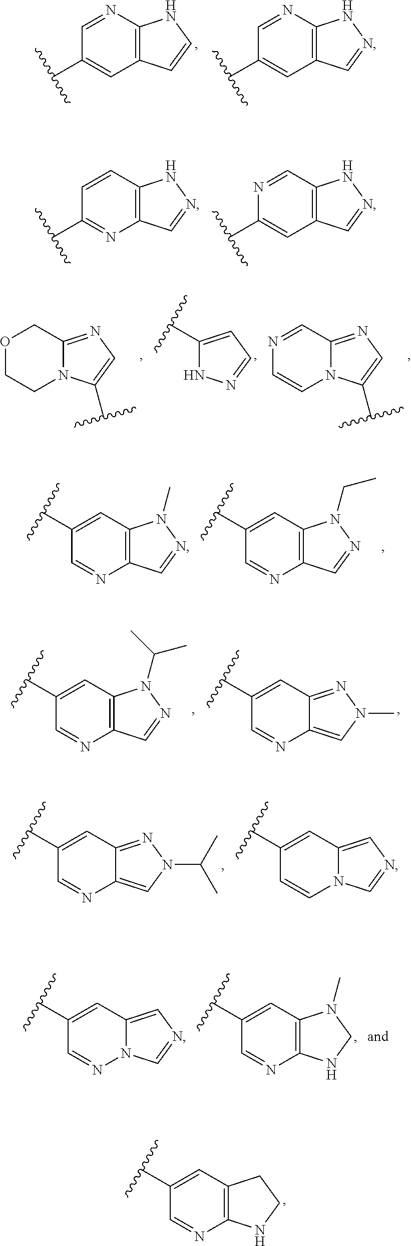

wherein C may be optionally substituted on one or more available carbons by one, two, three, or more independent R³ substituents selected from the group consisting of C₁₋₆alkyl, C₂₋₆alkynyl, C₃₋₆cycloalkyl, cyano, C₁₋₆alkoxyl, oxo, phenyl, —C(O)N(R^{A})(R^{B}), —N(R^{A})(R^{B}), 5-6 membered heterocyclyl, and 5-6 membered heteroaryl, wherein
the C₁₋₆alkyl may be optionally substituted on one or more available carbons by one, two, three, or more substituents each independently selected from halogen and phenyl;
if the 5-6 membered heterocyclyl contains a substitutable ring nitrogen atom, that ring nitrogen atom may be optionally substituted by C₁₋₆alkyl; and
R^{A} and R^{B} are independently, for each occurrence, hydrogen or C₁₋₆alkyl.

In some embodiments, C is selected from the group consisting of

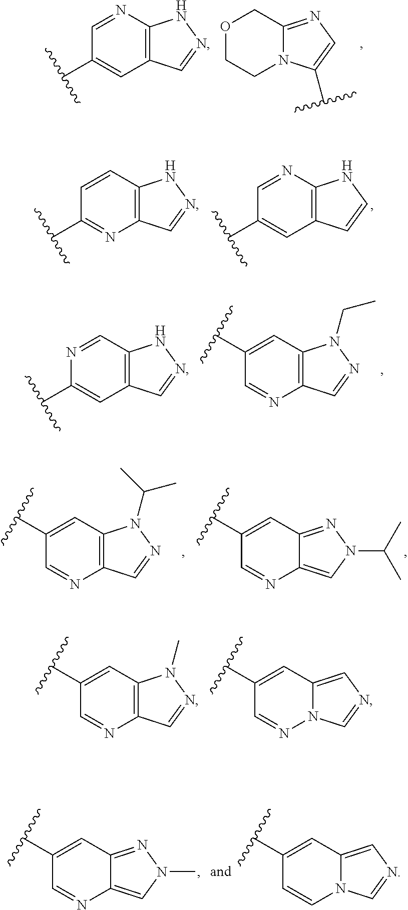

In some embodiments, C is selected from the group consisting of

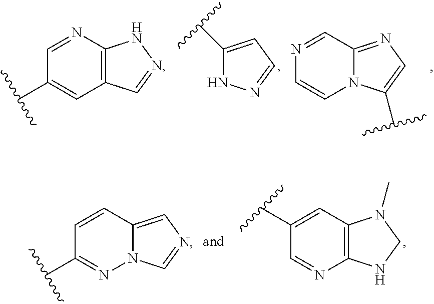

wherein C is substituted with one R³ substituent selected from the group consisting of C₁₋₆alkyl, C₂₋₆alkynyl, C₃₋₆cycloalkyl, cyano, C₁₋₆alkoxyl, oxo, phenyl, —C(O)N(R^{A})(R^{B}), and —N(R^{A})(R^{B}), wherein the C₁₋₆alkyl may be optionally substituted on one or more available carbons by one, two, three, or more independent halogen substituents and R^{A} and R^{B} are independently, for each occurrence, hydrogen or C₁₋₆alkyl.

In some embodiments, R³ is selected from the group consisting of cyano, oxo, CH₃, CH₂CH₃, C(H)(CH₃)₂, —CCH, CF₃, CH₂CHF₂, CF₂CH₃, CH₂CH₂F, —C(O)N(H)(CH₃), —O—CH₃, —NH₂, —N(H)(CH₃), cyclopropyl, and phenyl.

In some embodiments, C is selected from the group consisting of

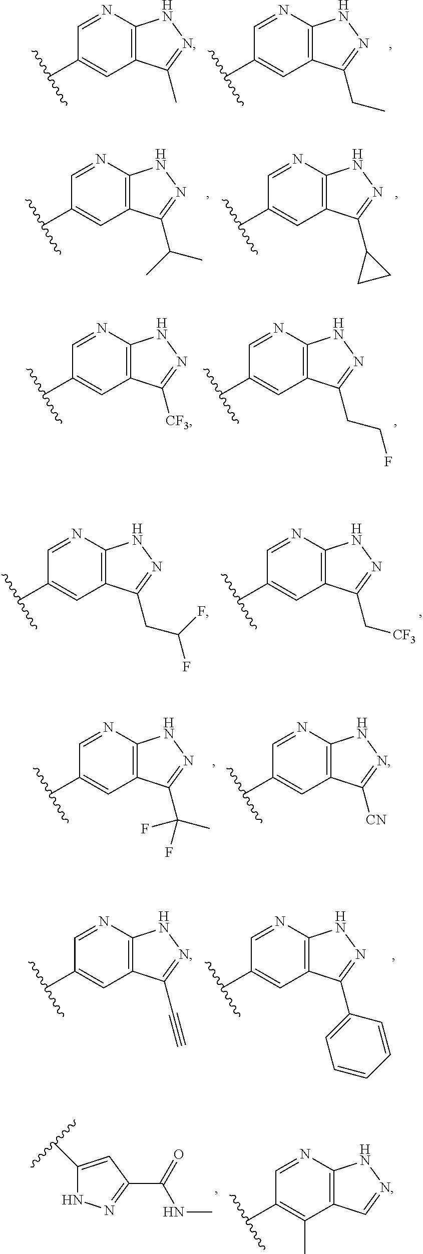

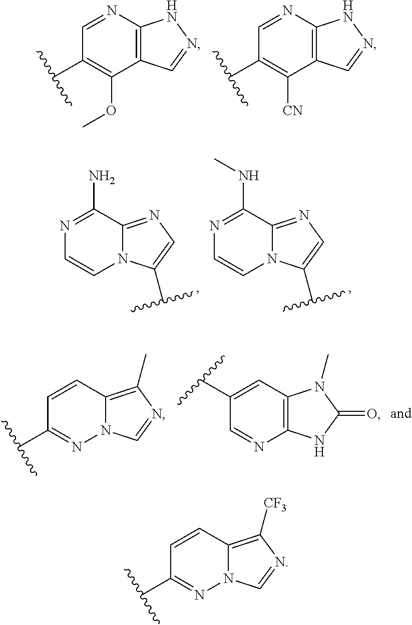

In some embodiments, C is

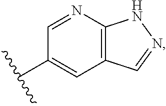

wherein C is substituted with two independent R³ substituents selected from the group consisting of C₁₋₆alkyl, C₂-6alkynyl, C₃₋₆cycloalkyl, cyano, C₁₋₆alkoxyl, oxo, phenyl, —C(O)N(R^{A})(R^{B}), —N(R^{A})(R^{B}), 5-6 membered heterocyclyl, and 5-6 membered heteroaryl, wherein
the C₁₋₆alkyl may be optionally substituted on one or more available carbons by one, two, three, or more substituents each independently selected from halogen and phenyl;
if the 5-6 membered heterocyclyl contains a substitutable ring nitrogen atom, that ring nitrogen atom may be optionally substituted by C₁₋₆alkyl; and
R^{A} and R^{B} are independently, for each occurrence, hydrogen or C₁₋₆alkyl.

In some embodiments, R³ is independently, for each occurrence, selected from the group consisting of CH₃, —CH(CH₃)₂, cyclopropyl, cyclopentyl, phenyl, pyridinyl,

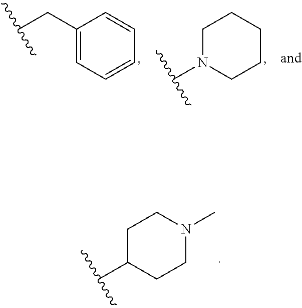

In some embodiments, R³ is independently, for each occurrence, selected from the group consisting of CH₃, —CH(CH₃)₂, cyclopropyl, cyclopentyl, phenyl,

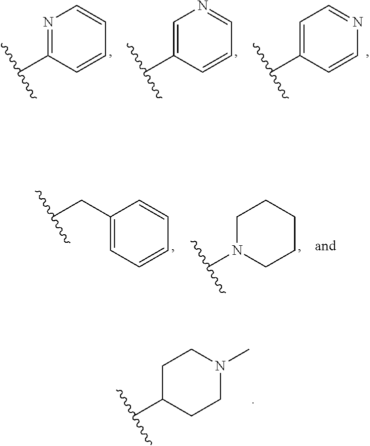

In some embodiments, R³ is independently, for each occurrence, CH₃ or phenyl.

In some embodiments, C is selected from the group consisting of

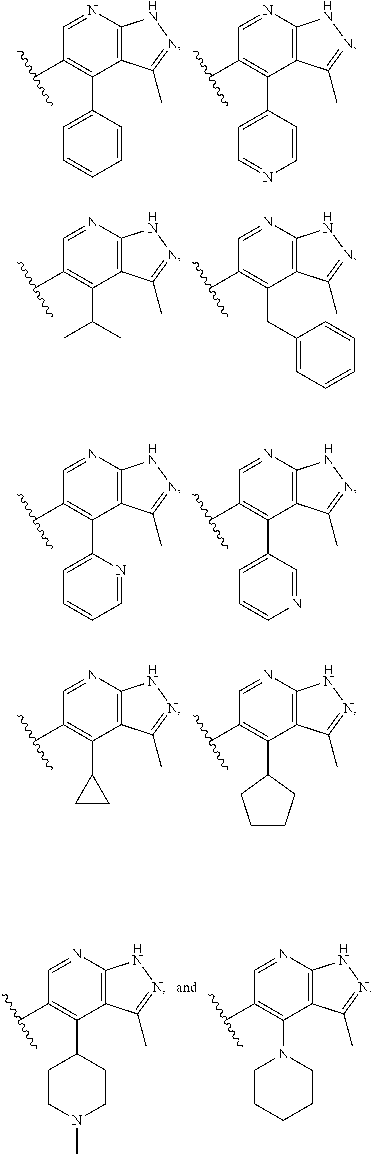

In some embodiments, C is

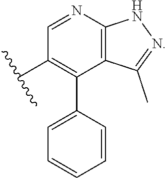 [SMILES: *c1cnc2[nH]nc(C)c2c1-c1ccccc1]

In some embodiments, C is

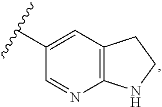 [SMILES: *c1cnc2c(c1)CCN2]

### C is

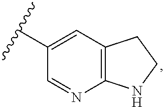

wherein C is substituted with three independent R³ substituents and wherein two of the R³ substituents together with the carbon to which they are attached form C₃₋₆cycloalkyl and the third R³ group is selected from the group consisting of C₁₋₆alkyl, C₂₋₆alkynyl, C₃₋₆cycloalkyl, cyano, C₁₋₆alkoxyl, oxo, phenyl, —C(O)N(R^{A})(R^{B}), and —N(R^{A})(R^{B}), wherein the C₁₋₆alkyl may be optionally substituted on one or more available carbons by one, two, three, or more independent halogen substituents and R^{A} and R^{B} are independently hydrogen or C₁₋₆alkyl.

In some embodiments, C is

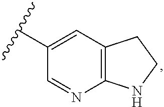

wherein C is substituted with three independent R³, and wherein two of the R³ substituents together with the carbon to which they are attached form cyclopropyl and the third R³ group is oxo.

In some embodiments, C is

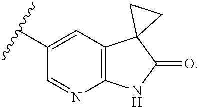 [SMILES: *c1cnc2c(c1)C1(CC1)C(=O)N2]

In some embodiments, C is selected from the group consisting of

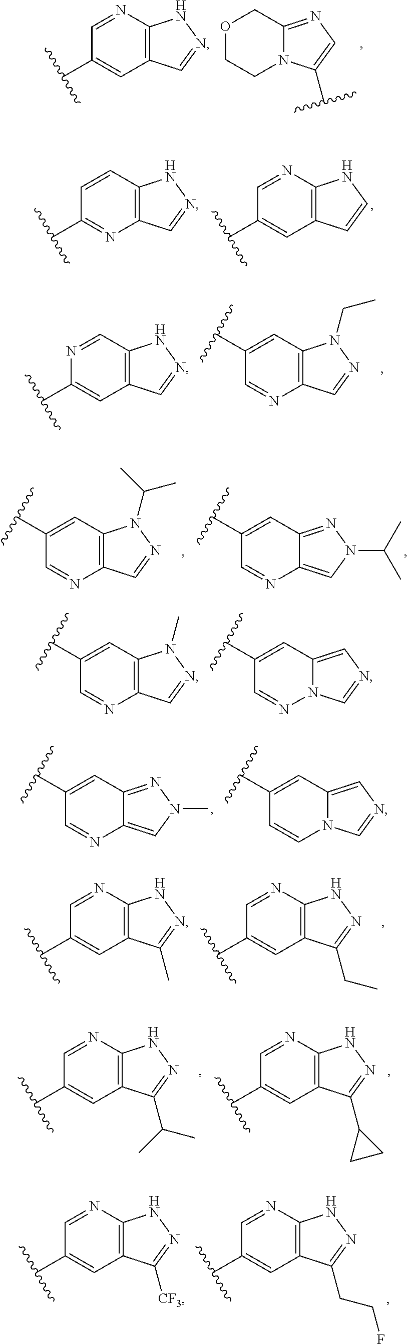

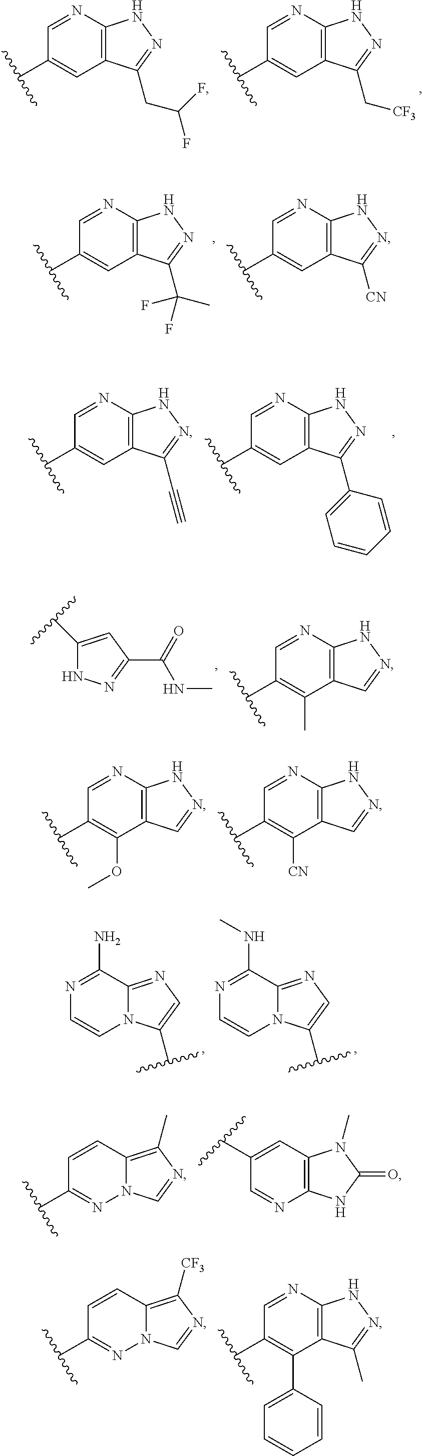

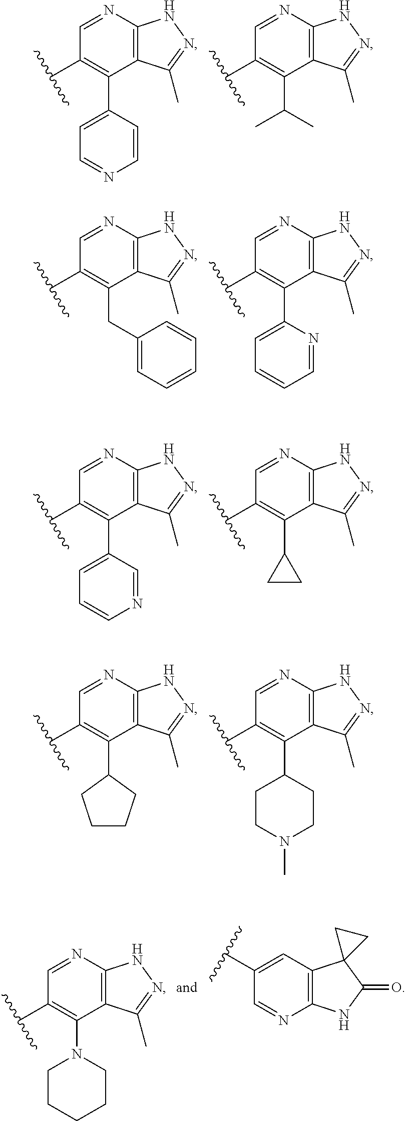

In another aspect, provided herein are compounds represented by Formula (Id):

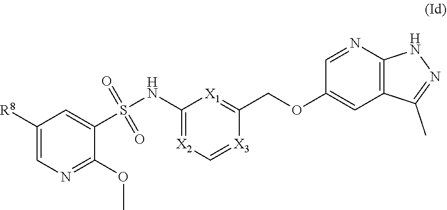

or a pharmaceutically acceptable salt thereof, wherein:
X₁ is N or CR⁹;
X₂ is N or CR⁹;
X₃ is N or CR⁹;
R⁸ is halogen; and
R⁹ is hydrogen or halogen.

In some embodiments, X₁ is CR⁹; X₂ is N; and X₃ is CR⁹. In some embodiments, R⁹ is chloro or fluoro.

In some embodiments, X₁ is CR⁹; X₂ is CR⁹; and X₃ is N. In some embodiments, R⁹ is hydrogen or fluoro.

In some embodiments, X₁ is N; X₂ is CR⁹; and X₃ is CR⁹. In some embodiments, R⁹ is fluoro.

In some embodiments, R⁸ is chloro or fluoro.

In another aspect, the compound is any compound set forth in Table 1, or a pharmaceutically acceptable salt thereof.

In another aspect, the compound is selected from the group consisting of
5-chloro-N-[2,4-difluoro-3-([1H-pyrazolo[3,4-b]pyridin-5-yloxy]methyl)phenyl]-2-methoxypyridine-3-sulfonamide;
N-[2,4-difluoro-3-[([3-methyl-1H-pyrazolo[3,4-b]pyridin-5-yl]oxy)methyl]phenyl]-5-fluoro-2-methoxypyridine-3-sulfonamide;
5-chloro-N-[2,4-difluoro-3-([1H-pyrazolo[3,4-b]pyridin-5-ylamino]methyl)phenyl]-2-methoxypyridine-3-sulfonamide;
5-chloro-N-(2,4-difluoro-3-[1H-pyrazolo[3,4-b]pyridin-5-ylmethoxy]phenyl)-2-methoxypyridine-3-sulfonamide;
5-chloro-N-[2,4-difluoro-3-(2-[5H,6H,8H-imidazo[2,1-c][1,4]oxazin-3-yl]ethyl)phenyl]-2-methoxypyridine-3-sulfonamide;
5-chloro-N-[2,4-difluoro-3-([1H-pyrazolo[3,4-b]pyridin-5-ylmethyl]amino)phenyl]-2-methoxypyridine-3-sulfonamide;
5-chloro-N-[2,4-difluoro-3-(2-[1H-pyrazolo[3,4-b]pyridin-5-yl]ethyl)phenyl]-2-methoxypyridine-3-sulfonamide;
5-[2-[3-(5-chloro-2-methoxypyridine-3-sulfonamido)-2,6-difluorophenyl]ethyl]-N-methyl-1H-pyrazole-3-carboxamide;
N-(3-(2-(1H-pyrazolo[3,4-b]pyridin-5-yl)ethyl)-2,4-difluorophenyl)-5-fluoro-2-methylpyridine-3-sulfonamide;
5-cyano-N-[2,4-difluoro-3-(2-[1H-pyrazolo[3,4-b]pyridin-5-yl]ethyl)phenyl]-2-methoxypyridine-3-sulfonamide;
5-chloro-N-[2,4-difluoro-3-(2-[1H-pyrazolo[4,3-b]pyridin-5-yl]ethyl)phenyl]-2-methoxypyridine-3-sulfonamide;
5-chloro-N-[2,4-difluoro-3-(2-[1H-pyrrolo[2,3-b]pyridin-5-yl]ethyl)phenyl]-2-methoxypyridine-3-sulfonamide;
5-chloro-N-[2,4-difluoro-3-(2-[1H-pyrazolo[3,4-c]pyridin-5-yl]ethyl)phenyl]-2-methoxypyridine-3-sulfonamide;
5-chloro-N-[2,4-difluoro-3-[([3-methyl-1H-pyrazolo[3,4-b]pyridin-5-yl]oxy)methyl]phenyl]-2-methoxypyridine-3-sulfonamide;
N-[2,4-difluoro-3-(2-[1H-pyrazolo[3,4-b]pyridin-5-yl]ethyl)phenyl]-5-fluoro-2-methoxypyridine-3-sulfonamide;
3-cyano-N-[2,4-difluoro-3-(2-[1H-pyrazolo[3,4-b]pyridin-5-yl]ethyl)phenyl]-5-(trifluoromethyl)benzenesulfonamide;
5-chloro-N-[2,4-difluoro-3-(2-[2-methylpyrazolo[4,3-b]pyridin-6-yl]ethyl)phenyl]-2-methoxypyridine-3-sulfonamide;
5-chloro-N-[2,4-difluoro-3-(2-[1-methylpyrazolo[4,3-b]pyridin-6-yl]ethyl)phenyl]-2-methoxypyridine-3-sulfonamide;
5-chloro-N-[2,4-difluoro-3-(2-[imidazo[1,5-a]pyridin-7-yl]ethyl)phenyl]-2-methoxypyridine-3-sulfonamide;
N-[2,4-difluoro-3-[([4-methyl-1H-pyrazolo[3,4-b]pyridin-5-yl]oxy)methyl]phenyl]-5-fluoro-2-methoxypyridine-3-sulfonamide;
N-[2,4-difluoro-3-([1H-pyrazolo[3,4-b]pyridin-5-yloxy]methyl)phenyl]-5-fluoro-2-methoxypyridine-3-sulfonamide;
5-chloro-N-[2,4-difluoro-3-[([4-methyl-1H-pyrazolo[3,4-b]pyridin-5-yl]oxy)methyl]phenyl]-2-methoxypyridine-3-sulfonamide;
N-[2,4-difluoro-3-[([4-methoxy-1H-pyrazolo[3,4-b]pyridin-5-yl]oxy)methyl]phenyl]-5-fluoro-2-methoxypyridine-3-sulfonamide;
5-chloro-N-[2,4-difluoro-3-[([4-methoxy-1H-pyrazolo[3,4-b]pyridin-5-yl]oxy)methyl]phenyl]-2-methoxypyridine-3-sulfonamide;
5-chloro-N-[2,4-difluoro-3-(1-[1H-pyrazolo[3,4-b]pyridin-5-yloxy]ethyl)phenyl]-2-methoxypyridine-3-sulfonamide;
5-cyano-N-[2,4-difluoro-3-([1H-pyrazolo[3,4-b]pyridin-5-yloxy]methyl)phenyl]-2-methoxypyridine-3-sulfonamide;
N-[3-[([4-cyano-1H-pyrazolo[3,4-b]pyridin-5-yl]oxy)methyl]-2,4-difluorophenyl]-5-fluoro-2-methoxypyridine-3-sulfonamide;
5-chloro-N-[3-[([4-cyano-1H-pyrazolo[3,4-b]pyridin-5-yl]oxy)methyl]-2,4-difluorophenyl]-2-methoxypyridine-3-sulfonamide;
5-chloro-N-[2,4-difluoro-3-(1-[1H-pyrazolo[3,4-b]pyridin-5-ylamino]ethyl)phenyl]-2-methoxypyridine-3-sulfonamide;
N-(3-(((1H-pyrazolo[3,4-b]pyridin-5-yl)oxy)methyl)-2,4-difluorophenyl)-5-fluoro-2-methylpyridine-3-sulfonamide;
5-chloro-N-(2,4-difluoro-3-(2-(8-(methylamino)imidazo[1,2-a]pyrazin-3-yl)ethyl)phenyl)-2-methoxypyridine-3-sulfonamide;
N-[3-(2-[8-aminoimidazo[1,2-a]pyrazin-3-yl]ethyl)-2,4-difluorophenyl]-5-chloro-2-methoxypyridine-3-sulfonamide;
5-chloro-N-[3-(2-[1-ethylpyrazolo[4,3-b]pyridin-6-yl]ethyl)-2,4-difluorophenyl]-2-methoxypyridine-3-sulfonamide;
N-[3-(2-[1-ethylpyrazolo[4,3-b]pyridin-6-yl]ethyl)-2,4-difluorophenyl]-5-fluoro-2-methoxypyridine-3-sulfonamide;
5-chloro-N-[2,4-difluoro-3-(2-[1-isopropylpyrazolo[4,3-b]pyridin-6-yl]ethyl)phenyl]-2-methoxypyridine-3-sulfonamide;
N-[2,4-difluoro-3-(2-[1-isopropylpyrazolo[4,3-b]pyridin-6-yl]ethyl)phenyl]-5-fluoro-2-methoxypyridine-3-sulfonamide;
N-[2,4-difluoro-3-(2-[2-isopropylpyrazolo[4,3-b]pyridin-6-yl]ethyl)phenyl]-5-fluoro-2-methoxypyridine-3-sulfonamide;
N-[2,4-difluoro-3-[([3-methyl-1H-pyrazolo[3,4-b]pyridin-5-yl]oxy)methyl]phenyl]-5-fluoro-2-methylpyridine-3-sulfonamide;
N-[3-[([3-ethyl-1H-pyrazolo[3,4-b]pyridin-5-yl]oxy)methyl]-2,4-difluorophenyl]-5-fluoro-2-methoxypyridine-3-sulfonamide;
5-chloro-N-[3-[([3-ethyl-1H-pyrazolo[3,4-b]pyridin-5-yl]oxy)methyl]-2,4-difluorophenyl]-2-methoxypyridine-3-sulfonamide;
5-cyano-N-[3-[([3-ethyl-1H-pyrazolo[3,4-b]pyridin-5-yl]oxy)methyl]-2,4-difluorophenyl]-2-methoxypyridine-3-sulfonamide;
5-chloro-N-[2,4-difluoro-3-[([1-methylpyrazolo[4,3-b]pyridin-6-yl]oxy)methyl]phenyl]-2-methoxypyridine-3-sulfonamide;
N-[2,4-difluoro-3-[([1-methylpyrazolo[4,3-b]pyridin-6-yl]oxy)methyl]phenyl]-5-fluoro-2-methoxypyridine-3-sulfonamide;
N-[2,4-difluoro-3-([[3-(trifluoromethyl)-1H-pyrazolo[3,4-b]pyridin-5-yl]oxy]methyl)phenyl]-5-fluoro-2-methoxypyridine-3-sulfonamide;
N-[2,4-difluoro-3-([3-methyl-1H-pyrazolo[3,4-b]pyridin-5-yl]methoxy)phenyl]-5-fluoro-2-methoxypyridine-3-sulfonamide;
5-chloro-N-[2,4-difluoro-3-([3-methyl-1H-pyrazolo[3,4-b]pyridin-5-yl]methoxy)phenyl]-2-methoxypyridine-3-sulfonamide;
N-[3-[([3-ethyl-1H-pyrazolo[3,4-b]pyridin-5-yl]oxy)methyl]-2,4-difluorophenyl]-5-fluoro-2-methylpyridine-3-sulfonamide; 5-cyano-N-(2,4-difluoro-3-((3-methyl-1H-pyrazolo[3,4-b]pyridin-5-yl)methoxy)phenyl)-2-methoxypyridine-3-sulfonamide;
5-cyano-N-[2,4-difluoro-3-[([3-methyl-1H-pyrazolo[3,4-b]pyridin-5-yl]methyl)amino]phenyl]-2-methoxypyridine-3-sulfonamide;
5-cyano-N-[2,4-difluoro-3-[([3-methyl-1H-pyrazolo[3,4-b]pyridin-5-yl]oxy)methyl]phenyl]-2-methoxypyridine-3-sulfonamide;
N-[2,4-difluoro-3-([3-methyl-1H-pyrazolo[3,4-b]pyridin-5-yl]methoxy)phenyl]-5-fluoro-2-methylpyridine-3-sulfonamide;
N-[2,4-difluoro-3-[([3-isopropyl-1H-pyrazolo[3,4-b]pyridin-5-yl]oxy)methyl]phenyl]-5-fluoro-2-methoxypyridine-3-sulfonamide;
5-chloro-N-[2,4-difluoro-3-[([3-isopropyl-1H-pyrazolo[3,4-b]pyridin-5-yl]oxy)methyl]phenyl]-2-methoxypyridine-3-sulfonamide;
N-[2,4-difluoro-3-[([3-isopropyl-1H-pyrazolo[3,4-b]pyridin-5-yl]oxy)methyl]phenyl]-5-fluoro-2-methylpyridine-3-sulfonamide;
5-cyano-N-[2,4-difluoro-3-[([3-isopropyl-1H-pyrazolo[3,4-b]pyridin-5-yl]oxy)methyl]phenyl]-2-methoxypyridine-3-sulfonamide;
N-[3,5-difluoro-4-[([3-methyl-1H-pyrazolo[3,4-b]pyridin-5-yl]oxy)methyl]pyridin-2-yl]-5-fluoro-2-methoxypyridine-3-sulfonamide;
N-[2,4-difluoro-3-[([3-methyl-1H-pyrazolo[3,4-b]pyridin-5-yl]methyl)amino]phenyl]-5-fluoro-2-methoxypyridine-3-sulfonamide;
5-chloro-N-[2,4-difluoro-3-[([3-methyl-1H-pyrazolo[3,4-b]pyridin-5-yl]methyl)amino]phenyl]-2-methoxypyridine-3-sulfonamide;
N-[2,4-difluoro-3-[([3-methyl-1H-pyrazolo[3,4-b]pyridin-5-yl]methyl)amino]phenyl]-5-fluoro-2-methylpyridine-3-sulfonamide;
5-chloro-N-(2,4-difluoro-3-((imidazo[1,5-b]pyridazin-3-yloxy)methyl)phenyl)-2-methoxypyridine-3-sulfonamide;
N-[2-chloro-4-fluoro-3-[([3-methyl-1H-pyrazolo[3,4-b]pyridin-5-yl]oxy)methyl]phenyl]-5-fluoro-2-methoxypyridine-3-sulfonamide;
N-[2-cyano-4-fluoro-3-[([3-methyl-1H-pyrazolo[3,4-b]pyridin-5-yl]oxy)methyl]phenyl]-5-fluoro-2-methoxypyridine-3-sulfonamide; 5-fluoro-N-[3-fluoro-2-[([3-methyl-1H-pyrazolo[3,4-b]pyridin-5-yl]oxy)methyl]pyridin-4-yl]-2-methoxypyridine-3-sulfonamide;
5-chloro-N-[3-fluoro-2-[([3-methyl-1H-pyrazolo[3,4-b]pyridin-5-yl]oxy)methyl]pyridin-4-yl]-2-methoxypyridine-3-sulfonamide;
5-chloro-N-[3,5-difluoro-4-[([3-methyl-1H-pyrazolo[3,4-b]pyridin-5-yl]oxy)methyl]pyridin-2-yl]-2-methoxypyridine-3-sulfonamide;
N-[3-chloro-5-fluoro-4-[([3-methyl-1H-pyrazolo[3,4-b]pyridin-5-yl]oxy)methyl]pyridin-2-yl]-5-fluoro-2-methoxypyridine-3-sulfonamide;
5-fluoro-N-[5-fluoro-6-[([3-methyl-1H-pyrazolo[3,4-b]pyridin-5-yl]oxy)methyl]pyridine-2-yl]-2-methoxypyridine-3-sulfonamide;
5-chloro-N-[5-fluoro-6-[([3-methyl-1H-pyrazolo[3,4-b]pyridin-5-yl]oxy)methyl]pyridin-2-yl]-2-methoxypyridine-3-sulfonamide;
N-[3-[([3-ethynyl-1H-pyrazolo[3,4-b]pyridin-5-yl]oxy)methyl]-2,4-difluorophenyl]-5-fluoro-2-methoxypyridine-3-sulfonamide;
N-(3-(((3-cyclopropyl-1H-pyrazolo[3,4-b]pyridin-5-yl)oxy)methyl)-2,4-difluorophenyl)-5-fluoro-2-methoxypyridine-3-sulfonamide;
N-[2,4-difluoro-3-[([1-methyl-2-oxo-3H-imidazo[4,5-b]pyridin-6-yl]oxy)methyl]phenyl]-5-fluoro-2-methoxypyridine-3-sulfonamide;
N-(2,4-difluoro-3-(((2′-oxo-1′,2′-dihydrospiro[cyclopropane-1,3′-pyrrolo[2,3-b]pyridin]-5′-yl)oxy)methyl)phenyl)-5-fluoro-2-methoxypyridine-3-sulfonamideN-(3-(((3-cyano-1H-pyrazolo[3,4-b]pyridin-5-yl)oxy)methyl)-2,4-difluorophenyl)-5-fluoro-2-methoxypyridine-3-sulfonamide;
N-[2-chloro-4-fluoro-3-[([3-methyl-1H-pyrazolo[3,4-b]pyridin-5-yl]oxy)methyl]phenyl]-5-fluoro-2-methylpyridine-3-sulfonamide;
N-[3-([[3-(2,2-difluoroethyl)-1H-pyrazolo[3,4-b]pyridin-5-yl]oxy]methyl)-2,4-difluorophenyl]-5-fluoro-2-methoxypyridine-3-sulfonamide;
N-[2,4-difluoro-3-([[3-(2-fluoroethyl)-1H-pyrazolo[3,4-b]pyridin-5-yl]oxy]methyl)phenyl]-5-fluoro-2-methoxypyridine-3-sulfonamide;
N-(4-chloro-2-fluoro-3-(((3-methyl-1H-pyrazolo[3,4-b]pyridin-5-yl)oxy)methyl)phenyl)-5-fluoro-2-methylpyridine-3-sulfonamide;
N-(4-chloro-2-fluoro-3-(((3-methyl-1H-pyrazolo[3,4-b]pyridin-5-yl)oxy)methyl)phenyl)-5-fluoro-2-methoxypyridine-3-sulfonamide;
5-chloro-N-[3-chloro-5-fluoro-4-[([3-methyl-1H-pyrazolo[3,4-b]pyridin-5-yl]oxy)methyl]pyridin-2-yl]-2-methoxypyridine-3-sulfonamide;
N-[2,4-difluoro-3-([[3-(2,2,2-trifluoroethyl)-1H-pyrazolo[3,4-b]pyridin-5-yl]oxy]methyl)phenyl]-5-fluoro-2-methoxypyridine-3-sulfonamide;
N-[3-([[3-(1,1-difluoroethyl)-1H-pyrazolo[3,4-b]pyridin-5-yl]oxy]methyl)-2,4-difluorophenyl]-5-fluoro-2-methoxypyridine-3-sulfonamide;
N-[2,4-difluoro-3-[([5-methylimidazo[1,5-b]pyridazin-3-yl]oxy)methyl]phenyl]-5-fluoro-2-methoxypyridine-3-sulfonamide;
N-[2,4-difluoro-3-([[5-(trifluoromethyl)imidazo[1,5-b]pyridazin-3-yl]oxy]methyl)phenyl]-5-fluoro-2-methoxypyridine-3-sulfonamide;
N-[2,4-difluoro-3-[([3-phenyl-1H-pyrazolo[3,4-b]pyridin-5-yl]oxy)methyl]phenyl]-5-fluoro-2-methoxypyridine-3-sulfonamide,
N-[2,4-difluoro-3-[([3-methyl-4-phenyl-1H-pyrazolo[3,4-b]pyridin-5-yl]oxy)methyl]phenyl]-5-fluoro-2-methoxypyridine-3-sulfonamide,
N-[3-[([4-cyclopropyl-3-methyl-1H-pyrazolo[3,4-b]pyridin-5-yl]oxy)methyl]-2,4-difluorophenyl]-5-fluoro-2-methoxypyridine-3-sulfonamide,
N-[2,4-difluoro-3-([[3-methyl-4-(pyridin-4-yl)-1H-pyrazolo[3,4-b]pyridin-5-yl]oxy]methyl)phenyl]-5-fluoro-2-methoxypyridine-3-sulfonamide,
N-[2,4-difluoro-3-[([4-isopropyl-3-methyl-1H-pyrazolo[3,4-b]pyridin-5-yl]oxy)methyl]phenyl]-5-fluoro-2-methoxypyridine-3-sulfonamide,
N-[3-[([4-benzyl-3-methyl-1H-pyrazolo[3,4-b]pyridin-5-yl]oxy)methyl]-2,4-difluorophenyl]-5-fluoro-2-methoxypyridine-3-sulfonamide,
N-[2,4-difluoro-3-([[3-methyl-4-(pyridin-2-yl)-1H-pyrazolo[3,4-b]pyridin-5-yl]oxy]methyl)phenyl]-5-fluoro-2-methoxypyridine-3-sulfonamide,
N-[2,4-difluoro-3-([[3-methyl-4-(pyridin-3-yl)-1H-pyrazolo[3,4-b]pyridin-5-yl]oxy]methyl)phenyl]-5-fluoro-2-methoxypyridine-3-sulfonamide,
N-[3-[([4-cyclopentyl-3-methyl-1H-pyrazolo[3,4-b]pyridin-5-yl]oxy)methyl]-2,4-difluorophenyl]-5-fluoro-2-methoxypyridine-3-sulfonamide,
N-[2,4-difluoro-3-([[3-methyl-4-(1-methylpiperidin-4-yl)-1H-pyrazolo[3,4-b]pyridin-5-yl]oxy]methyl)phenyl]-5-fluoro-2-methoxypyridine-3-sulfonamide, and
N-[2,4-difluoro-3-([[3-methyl-4-(piperidin-1-yl)-1H-pyrazolo[3,4-b]pyridin-5-yl]oxy]methyl)phenyl]-5-fluoro-2-methoxypyridine-3-sulfonamide,
   or a pharmaceutically acceptable salt thereof.

### Therapeutic Applications of GCN2 Modulating (Inhibiting/Activating) Compounds

It is contemplated that GCN2 modulating (inhibiting/activating) compounds and related compounds described herein, such as a compound of Formula I, provide therapeutic benefits to subjects suffering from cancer, neurodegenerative disease, and doxorubicin-induced cardiotoxicity. Accordingly, one aspect of the invention provides therapeutic methods for treating the foregoing diseases and conditions using GCN2 modulating compounds and related compounds described herein. Various aspects and embodiments of the therapeutic methods are described below.

### Cancer

One aspect of the invention provides a method of treating cancer in a subject. The method comprises administering a therapeutically effective amount of a GCN2 modulating (inhibiting/activating) compound or related compound described herein, such as a compound of Formula 1 to a subject in need thereof to treat the cancer. In certain embodiments, the particular compound of Formula I, is a compound defined by one of the embodiments described above.

In certain embodiments, the cancer is a solid tumor, leukemia, or lymphoma. In certain embodiments, the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, sweat gland carcinoma, sebaceous gland carcinoma, thyroid cancer, kidney cancer, uterus cancer, esophagus cancer, liver cancer, head cancer, neck cancer, throat cancer, mouth cancer, bone cancer, chest cancer, lymph node cancer, eye cancer, mesothelioma, an acoustic neuroma, oligodendroglioma, meningioma, neuroblastoma, retinoblastoma, leukemia, or lymphoma. In certain embodiments, the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, leukemia, or lymphoma. In certain other embodiments, the cancer is colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, lung cancer, leukemia, bladder cancer, stomach cancer, cervical cancer, testicular cancer, skin cancer, rectal cancer, thyroid cancer, kidney cancer, uterus cancer, esophagus cancer, liver cancer, an acoustic neuroma, oligodendroglioma, meningioma, neuroblastoma, or retinoblastoma. In certain other embodiments, the cancer is small cell lung cancer, non-small cell lung cancer, melanoma, cancer of the central nervous system tissue, brain cancer, Hodgkin's lymphoma, non-Hodgkin's lymphoma, cutaneous T-Cell lymphoma, cutaneous B-Cell lymphoma, or diffuse large B-Cell lymphoma. In certain other embodiments, the cancer is breast cancer, colon cancer, small-cell lung cancer, non-small cell lung cancer, prostate cancer, renal cancer, ovarian cancer, leukemia, melanoma, or cancer of the central nervous system tissue. In certain other embodiments, the cancer is colon cancer, small-cell lung cancer, non-small cell lung cancer, renal cancer, ovarian cancer, renal cancer, or melanoma.

Additional exemplary cancers include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, and hemangioblastoma.

In certain embodiments, the cancer is a neuroblastoma, meningioma, hemangiopericytoma, multiple brain metastases, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, sweat gland carcinoma, sebaceous gland carcinoma, thyroid cancer, kidney cancer, uterus cancer, esophagus cancer, liver cancer, head cancer, neck cancer, throat cancer, mouth cancer, bone cancer, chest cancer, lymph node cancer, eye cancer, mesothelioma, an acoustic neuroma, oligodendroglioma, meningioma, neuroblastoma, retinoblastoma, leukemia, or lymphoma.

In certain embodiments, the cancer is colon cancer, pancreatic cancer, breast cancer, ovarian cancer, prostate cancer, squamous cell carcinoma, basal cell carcinoma, adenocarcinoma, lung cancer, bladder cancer, stomach cancer, cervical cancer, testicular cancer, skin cancer, rectal cancer, leukemia, or lymphoma.

### Neurodegenerative Disease

Another aspect of the invention provides a method of treating a neurodegenerative disease in a subject. The method comprises administering a therapeutically effective amount of a compound described herein, such as a compound of Formula I, to a subject in need thereof to treat the neurodegenerative disease. In certain embodiments, the neurodegenerative disease is Alzheimer's disease, Parkinson's Disease, Huntington's Disease, amyotrophic lateral sclerosis, or spinocerebellar ataxia.

Aberrant autophagic processes contribute to neurodegenerative diseases. For example, γ-secretase activity is enhanced in autophagic vacuoles through signal transduction mediated by GCN2 phosphorylation of the a subunit of eukaryotic initiation factor 2 (eIF2α) (see, e.g., Ohta, K. et al. in Autophagy 2010, 6, 345-352). The γ-secretase enhances amyloid-β synthesis and the progression of Alzheimer's disease. Accordingly, compounds having inhibitory activity towards GCN2 provide benefits to patients suffering from neurodegenerative diseases.

### Doxorubicin-Induced Cardiotoxicity

Another aspect of the invention provides a method of treating doxorubicin-induced cardiotoxicity in a subject. The method comprises administering a therapeutically effective amount of a compound described herein, such as a compound of Formula I, to a subject in need thereof suffering from doxorubicin-induced cardiotoxicity, to thereby treat the doxorubicin-induced cardiotoxicity.

Another aspect of the invention provides a method of preventing doxorubicin-induced cardiotoxicity in a subject. The method comprises administering a therapeutically effective amount of a compound described herein, such as a compound of Formula I, to a subject in need thereof that has received, or will receive, doxorubicin, to thereby prevent doxorubicin-induced cardiotoxicity.

Deficiency in GCN2 has been reported to ameliorate doxorubicin-induced cardiotoxicity. See, for example, Wang et al. in Redox Biology (2018) vol. 17, pages 25-34. Accordingly, compounds having inhibitory activity towards GCN2 provide benefits to patients suffering from or likely to suffer from doxorubicin-induced cardiotoxicity.

In certain embodiments, the subject is a human.

Another aspect of the invention provides for the use of a compound described herein (such as a compound of Formula I) in the manufacture of a medicament. In certain embodiments, the medicament is for treating a disorder described herein, such as cancer.

Another aspect of the invention provides for the use of a compound described herein (such as a compound of Formula I) for treating a medical disorder, such a medical disorder described herein (e.g., cancer).

Further, it is contemplated that GCN2 modulators (inhibitors/activators) and related compounds described herein, such as a compound of Formula I, can inhibit/activate the activity of GCN2. Accordingly, another aspect of the invention provides a method of inhibiting/activating the activity of GCN2. The method comprises exposing a GCN2 to an effective amount of a GCN2 modulator (inhibitor/activator) or related compound described herein, such as a compound of Formula I, to inhibit/activate GCN2 activity. In certain embodiments, the particular compound of Formula I, is the compound defined by one of the embodiments described above.

### Combination Therapy

Another aspect of the invention provides for combination therapy. GCN2 modulators (inhibitors/activators) and related compounds (e.g., a compound of Formula I) or their pharmaceutically acceptable salts may be used in combination with additional therapeutic agents to treat medical disorders, such as a cancer.

Exemplary therapeutic agents that may be used as part of a combination therapy in treating cancer, include, for example, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

Radiation therapy may also be used as part of a combination therapy.

An additional class of agents that may be used as part of a combination therapy in treating cancer is immune checkpoint inhibitors (also referred to as immune checkpoint blockers). Immune checkpoint inhibitors are a class of therapeutic agents that have the effect of blocking immune checkpoints. See, for example, Pardoll in Nature Reviews Cancer (2012) vol. 12, pages 252-264. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAB3, (v) B7-H3, (vi) B7-H4, and (vii) TIM3. The CTLA4 inhibitor Ipilumumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytoxic agents (e.g., tyrosine-kinase inhibitors).

Yet other agents that may be used as part of a combination therapy in treating cancer are agents which deplete amino acids or other nutrients, radiation, and agents that provoke the integrated stress response or that promote autophagy. Such agents may include aspariginase, argininase inhibitors of kinases such a b-Raf, and cytotoxic agents such as cis-platin.

Accordingly, another aspect of the invention provides a method of treating cancer in a patient, where the method comprises administering to the patient in need thereof (i) a therapeutically effective amount of a GCN2 modulator (activator/inhibitor) compound described herein and (ii) a second anti-cancer agent, in order to treat the cancer, where the second therapeutic agent may be one of the additional therapeutic agents described above (e.g., mitomycin, tretinoin, ribomustin, gemcitabine, an immune checkpoint inhibitor, or a monoclonal antibody agent that targets non-checkpoint targets) or one of the following:
an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor;
an agonist of OX40, CD137, CD40, GITR, CD27, HVEM, TNFRSF25, or ICOS;
a therapeutic antibody targeting one of the following: CD20, CD30, CD33, CD52, EpCAM, CEA, gpA33, a mucin, TAG-72, CAIX, PSMA, a folate-binding protein, a ganglioside, Le, VEGF, VEGFR, VEGFR2, integrin αVβ3, integrin α5β1, EGFR, ERBB2, ERBB3, MET, IGF1R, EPHA3, TRAILR1, TRAILR2, RANKL, FAP, tenascin, CD19, KIR, NKG2A, CD47, CEACAM1, c-MET, VISTA, CD73, CD38, BAFF, interleukin-1 beta, B4GALNT1, interleukin-6, and interleukin-6 receptor;
a cytokine selected from IL-12, IL-15, GM-CSF, and G-CSF;
a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine; or
a placental growth factor, an antibody-drug conjugate, an oncolytic virus, or an anti-cancer vaccine.

In certain embodiments, the second anti-cancer agent is an ALK Inhibitor. In certain embodiments, the second anti-cancer agent is an ALK Inhibitor comprising ceritinib or crizotinib. In certain embodiments, the second anti-cancer agent is an ATR Inhibitor. In certain embodiments, the second anti-cancer agent is an ATR Inhibitor comprising AZD6738 or VX-970. In certain embodiments, the second anti-cancer agent is an A2A Antagonist. In certain embodiments, the second anti-cancer agent is a Base Excision Repair Inhibitor comprising methoxyamine. In certain embodiments, the second anti-cancer agent is a Base Excision Repair Inhibitor, such as methoxyamine. In certain embodiments, the second anti-cancer agent is a Bcr-Abl Tyrosine Kinase Inhibitor. In certain embodiments, the second anti-cancer agent is a Bcr-Abl Tyrosine Kinase Inhibitor comprising dasatinib or nilotinib. In certain embodiments, the second anti-cancer agent is a Bruton's Tyrosine Kinase Inhibitor. In certain embodiments, the second anti-cancer agent is a Bruton's Tyrosine Kinase Inhibitor comprising ibrutinib. In certain embodiments, the second anti-cancer agent is a CDC7 Inhibitor. In certain embodiments, the second anti-cancer agent is a CDC7 Inhibitor comprising RXDX-103 or AS-141.

In certain embodiments, the second anti-cancer agent is a CHK1 Inhibitor. In certain embodiments, the second anti-cancer agent is a CHK1 Inhibitor comprising MK-8776, ARRY-575, or SAR-020106. In certain embodiments, the second anti-cancer agent is a Cyclin-Dependent Kinase Inhibitor. In certain embodiments, the second anti-cancer agent is a Cyclin-Dependent Kinase Inhibitor comprising palbociclib. In certain embodiments, the second anti-cancer agent is a DNA-PK Inhibitor. In certain embodiments, the second anti-cancer agent is a DNA-PK Inhibitor comprising MSC2490484A. In certain embodiments, the second anti-cancer agent is Inhibitor of both DNA-PK and mTOR. In certain embodiments, the second anti-cancer agent comprises CC-115.

In certain embodiments, the second anti-cancer agent is a DNMT1 Inhibitor. In certain embodiments, the second anti-cancer agent is a DNMT1 Inhibitor comprising decitabine, RX-3117, guadecitabine, NUC-8000, or azacytidine. In certain embodiments, the second anti-cancer agent comprises a DNMT1 Inhibitor and 2-chloro-deoxyadenosine. In certain embodiments, the second anti-cancer agent comprises ASTX-727.

In certain embodiments, the second anti-cancer agent is a HDAC Inhibitor. In certain embodiments, the second anti-cancer agent is a HDAC Inhibitor comprising OBP-801, CHR-3996, etinostate, resminostate, pracinostat, CG-200745, panobinostat, romidepsin, mocetinostat, belinostat, AR-42, ricolinostat, KA-3000, or ACY-241.

In certain embodiments, the second anti-cancer agent is a Hedgehog Signaling Pathway Inhibitor. In certain embodiments, the second anti-cancer agent is a Hedgehog Signaling Pathway Inhibitor comprising sonidegib or vismodegib. In certain embodiments, the second anti-cancer agent is an IDO Inhibitor. In certain embodiments, the second anti-cancer agent is an IDO Inhibitor comprising INCB024360. In certain embodiments, the second anti-cancer agent is a JAK Inhibitor. In certain embodiments, the second anti-cancer agent is a JAK Inhibitor comprising ruxolitinib or tofacitinib. In certain embodiments, the second anti-cancer agent is a mTOR Inhibitor. In certain embodiments, the second anti-cancer agent is a mTOR Inhibitor comprising everolimus or temsirolimus. In certain embodiments, the second anti-cancer agent is a MEK Inhibitor. In certain embodiments, the second anti-cancer agent is a MEK Inhibitor comprising cobimetinib or trametinib. In certain embodiments, the second anti-cancer agent is a MELK Inhibitor. In certain embodiments, the second anti-cancer agent is a MELK Inhibitor comprising ARN-7016, APTO-500, or OTS-167. In certain embodiments, the second anti-cancer agent is a MTH1 Inhibitor. In certain embodiments, the second anti-cancer agent is a MTH1 Inhibitor comprising (S)-crizotinib, TH287, or TH588.

In certain embodiments, the second anti-cancer agent is a PARP Inhibitor. In certain embodiments, the second anti-cancer agent is a PARP Inhibitor comprising MP-124, olaparib, BGB-290, talazoparib, veliparib, niraparib, E7449, rucaparb, or ABT-767. In certain embodiments, the second anti-cancer agent is a Phosphoinositide 3-Kinase Inhibitor. In certain embodiments, the second anti-cancer agent is a Phosphoinositide 3-Kinase Inhibitor comprising idelalisib. In certain embodiments, the second anti-cancer agent is an inhibitor of both PARP1 and DHODH (i.e., an agent that inhibits both poly ADP ribose polymerase 1 and dihydroorotate dehydrogenase).

In certain embodiments, the second anti-cancer agent is a Proteasome Inhibitor. In certain embodiments, the second anti-cancer agent is a Proteasome Inhibitor comprising bortezomib or carfilzomib. In certain embodiments, the second anti-cancer agent is a Topoisomerase-II Inhibitor. In certain embodiments, the second anti-cancer agent is a Topoisomerase-II Inhibitor comprising vosaroxin.

In certain embodiments, the second anti-cancer agent is a Tyrosine Kinase Inhibitor. In certain embodiments, the second anti-cancer agent is a Tyrosine Kinase Inhibitor comprising bosutinib, cabozantinib, imatinib or ponatinib. In certain embodiments, the second anti-cancer agent is a VEGFR Inhibitor. In certain embodiments, the second anti-cancer agent is a VEGFR Inhibitor comprising regorafenib. In certain embodiments, the second anti-cancer agent is a WEE1 Inhibitor. In certain embodiments, the second anti-cancer agent is a WEE1 Inhibitor comprising AZD1775.

In certain embodiments, the second anti-cancer agent is an agonist of OX40, CD137, CD40, GITR, CD27, HVEM, TNFRSF25, or ICOS. In certain embodiments, the second anti-cancer agent is a therapeutic antibody selected from the group consisting of rituximab, ibritumomab tiuxetan, tositumomab, obinutuzumab, ofatumumab, brentuximab vedotin, gemtuzumab ozogamicin, alemtuzumab, IGN101, adecatumumab, labetuzumab, huA33, pemtumomab, oregovomab, minetumomab, cG250, J591, Mov18, farletuzumab, 3F8, ch14.18, KW-2871, hu3S193, lgN311, bevacizumab, IM-2C₆, pazopanib, sorafenib, axitinib, CDP791, lenvatinib, ramucirumab, etaracizumab, volociximab, cetuximab, panitumumab, nimotuzumab, 806, afatinib, erlotinib, gefitinib, osimertinib, vandetanib, trastuzumab, pertuzumab, MM-121, AMG 102, METMAB, SCH 900105, AVE1642, IMC-A12, MK-0646, R1507, CP 751871, KB004, IIIA-4, mapatumumab, HGS-ETR2, CS-1008, denosumab, sibrotuzumab, F19, 81C6, MEDI551, lirilumab, MEDI9447, daratumumab, belimumab, canakinumab, dinutuximab, siltuximab, and tocilizumab.

In certain embodiments, the second anti-cancer agent is a placental growth factor. In certain embodiments, the second anti-cancer agent is a placental growth factor comprising ziv-aflibercept. In certain embodiments, the second anti-cancer agent is an antibody-drug conjugate. In certain embodiments, the second anti-cancer agent is an antibody-drug conjugate selected from the group consisting of brentoxumab vedotin and trastuzumab emtransine.

In certain embodiments, the second anti-cancer agent is an oncolytic virus. In certain embodiments, the second anti-cancer agent is the oncolytic virus talimogene laherparepvec. In certain embodiments, the second anti-cancer agent is an anti-cancer vaccine. In certain embodiments, the second anti-cancer agent is an anti-cancer vaccine selected from the group consisting of a GM-CSF tumor vaccine, a STING/GM-CSF tumor vaccine, and NY-ESO-1. In certain embodiments, the second anti-cancer agent is a cytokine selected from IL-12, IL-15, GM-CSF, and G-CSF.

In certain embodiments, the second anti-cancer agent is a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine.

The doses and dosage regimen of the active ingredients used in the combination therapy may be determined by an attending clinician. In certain embodiments, the GCN2 modulator (inhibitor/activator) or related compound (e.g., a compound of any one of Formula I) and the additional therapeutic agent(s) are administered in doses commonly employed when such agents are used as monotherapy for treating the disorder. In other embodiments, the GCN2 modulator (inhibitor/activator) or related compound (e.g., a compound of any one of Formula I) and the additional therapeutic agent(s) are administered in doses lower than the doses commonly employed when such agents are used as monotherapy for treating the disorder. In certain embodiments, GCN2 modulator (inhibitor/activator) or related compound (e.g., a compound of any one of Formula I) and the additional therapeutic agent(s) are present in the same composition, which is suitable for oral administration.

In certain embodiments, GCN2 modulator (inhibitor/activator) or related compound (e.g., a compound of any one of Formula I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of the GCN2 modulator (inhibitor/activator) or related compound (e.g., a compound of any one of Formula I), a pharmaceutically acceptable carrier, vehicle or diluent, and optionally at least one additional therapeutic agent listed above.

### Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis), powders, granules, or as a solution or a suspension in an aqueous or non-aqueous liquid, or as an oil-in-water or water-in-oil liquid emulsion, or as an elixir or syrup, or as pastilles (using an inert base) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers and/or any of the following: (1) fillers or extenders; (2) binders; (3) humectants; (4) disintegrating agents; (5) solution retarding agents; (6) absorption accelerators, such as quaternary ammonium compounds and surfactants; (7) wetting agents; (8) absorbents; (9) lubricants; (10) coloring agents; and (11) controlled release agents. In the case of capsules, tablets and pills, the pharmaceutical compositions may also comprise buffering agents.

A tablet may be made by compression or molding, optionally with one or more accessory ingredients. Compressed tablets may be prepared using one or more binders, lubricants, inert diluents, preservatives, disintegrants, and/or surface-active or dispersing agents. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents.

Formulations of the pharmaceutical compositions of the invention for rectal or vaginal administration may be presented as a suppository, which may be prepared by mixing one or more compounds of the invention with one or more suitable nonirritating excipients or carriers which are solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Proper fluidity of the pharmaceutical compositions can be maintained, for example, by the use of coating materials, by the maintenance of the required particle size in the case of dispersions, and/or by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents. It may also be desirable to include isotonic agents into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers. Depending on the ratio of drug to polymer, and the nature of the particular polymer employed, the rate of drug release can be controlled. Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising an (aza)indazolyl-aryl sulfonamide or related compound described herein in a therapeutically effective amount for the treatment of a medical disorder described herein.

### EXAMPLES

The representative examples that follow are intended to help illustrate the invention, and are not intended to, nor should they be construed to, limit the scope of the invention.

The compounds provided herein can be prepared from readily available starting materials using the following general methods and procedures. It will be appreciated that where

typical or preferred process conditions (i.e., reaction temperatures, times, mole ratios of reactants, solvents, pressures, etc.) are given, other process conditions can also be used unless otherwise stated. Optimal reaction conditions may vary with the particular reactants or solvent
used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include recrystallization, filtration, flash chromatography, trituration, high pressure liquid chromatography (HPLC), or supercritical fluid chromatography

(SFC). Note that flash chromatography may either be performed manually or via an automated
system. The compounds provided herein may be characterized by known standard procedures,
such as nuclear magnetic resonance spectroscopy (NMR) or liquid chromatography mass spectrometry (LCMS). NMR chemical shifts are reported in part per million (ppm) and are generated using methods well known to those of skill in the art.

### LIST OF ABBREVIATIONS

Ac acetyl
ACN acetonitrile
AcOH acetic acid
AIBN azobisisobutyronitrile
BAST bis(2-methoxyethyl)aminosulfur trifluoride
9-BBN 9-Borabicyclo[3.3.1]nonane
Boc tert-butyloxycarbonyl
BINAP 2,2′-bis(diphenylphosphino)-1,1′-binaphthyl
BINOL 1,1′-bi-2-naphthol
BPO benzoyl peroxide
CDI carbonyldiimidazole
DAST diethylaminosulfur trifluoride
dba dibenzylideneacetone
DBU 1,8-diazabicyclo[5.4.0]undec-7-ene
DCE dichloroethane
DCM dichloromethane
DDQ 2,3-dichloro-5,6-dicyano-1,4-benzoquinone
DEAD Diethyl azodicarboxylate
DIEA diisopropylethylamine
DMA dimethylacetamide
DMAP 4-dimethylaminopyridine
DMF dimethylformamide
DMSO dimethyl sulfoxide
dppf 1,1′-bis(diphenylphosphino)ferrocene
EA ethyl acetate
EDTA Ethylenediaminetetraacetic acid
EtOAc ethyl acetate
FA formic acid
HATU 1-[Bis(dimethylamino)methylene]-1H-1,2,3-triazolo[4,5-b]pyridinium-3-oxide hexafluorophosphate
HPLC high performance liquid chromatography
IBX 2-iodoxybenzoic acid
IPA isopropyl alcohol
KOAc potassium acetate
LAH lithium aluminum hydride
LCMS liquid chromatography-mass spectrometry
LDA lithium diisopropylamide
MeCN acetonitrile
MTBE methyl tert-butyl ether
NBS N-bromosuccinimide
NCS N-chlorosuccinimide
NIS N-iodosuccinimide
NMR nuclear magnetic resonance spectroscopy
PCy₃ tricyclohexylphosphine
PE petroleum ether
Pin pinacolato
PMB p-methoxybenzyl
Py pyridine
SEM trimethylsilylethoxymethyl
TBAF tetrabutylammonium fluoride
TBHP tert-butyl hydroperoxide
TBS tert-butyldimethylsilyl
TEA triethylamine
Tf triflate/trifluoromethanesulfonate
TFA trifluoroacetic acid
TFAA trifluoroacetic anhydride
TMAD N,N,N′,N′-Tetramethylazodicarboxamide

### General Schemes

In some embodiments, compounds of the present disclosure may be manufactured using a process comprising one or more of Schemes 1-9 as set out below. Reaction steps represented by dashed arrows are to be understood to be optional. Unless otherwise specified, the variables of the Schemes are as defined herein. Reaction conditions should be understood to be exemplary and non-limiting, and may occur in the presence of an appropriate solvent.

In some embodiments, compounds of the present disclosure may be manufactured using a process comprising Scheme 1, wherein Z is halide, Y is amino or a functional group that may be readily converted to amino (e.g., nitro), and R^{a} is hydrogen or methyl when q is 1; p is 0 or 1, q is 0 or 1, and the sum of p and q is 1.

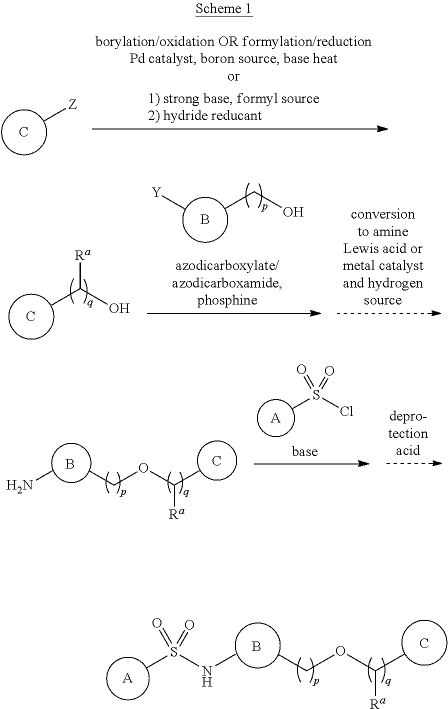

In some embodiments, compounds of the present disclosure may be manufactured using a process comprising Scheme 2, wherein Z is halide and Y is halide or hydroxyl.

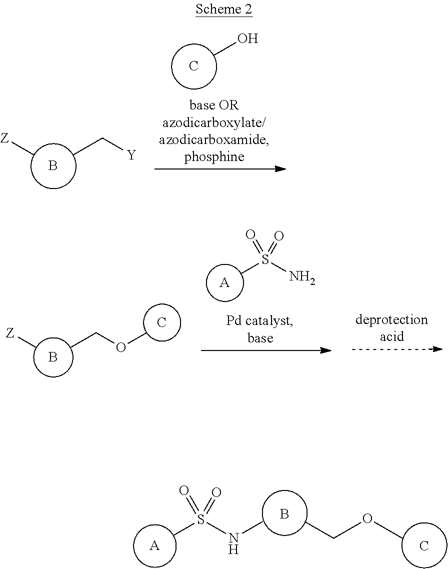

In some embodiments, compounds of the present disclosure may be manufactured using a process comprising Scheme 3, wherein Z is a functional group that may be readily converted to amino (e.g., nitro) and Y is halide or hydroxyl.

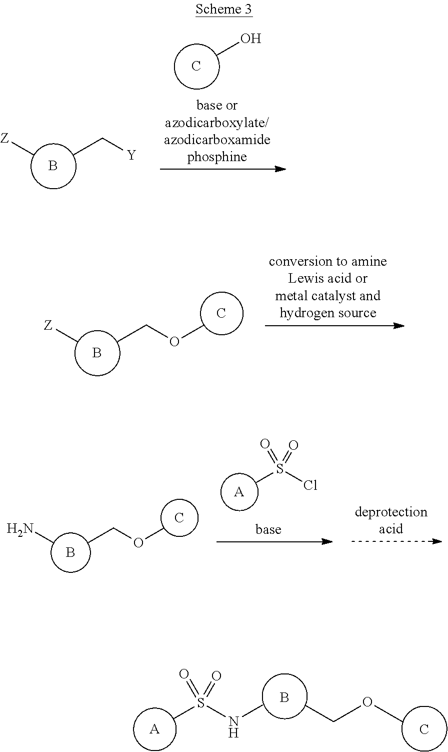

In some embodiments, compounds of the present disclosure may be manufactured using a process comprising Scheme 4, wherein Z is halide.

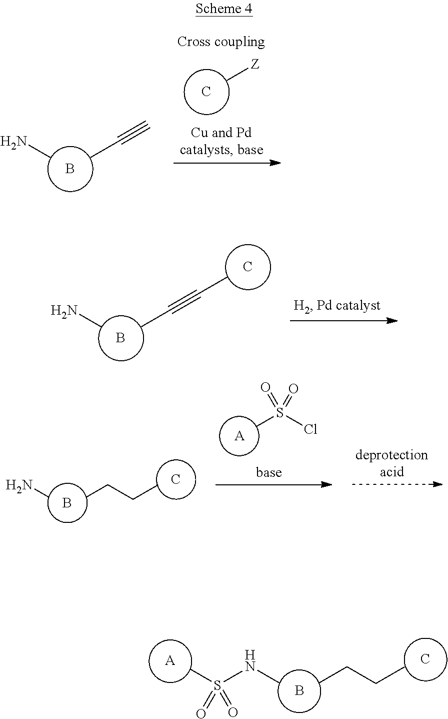

In some embodiments, compounds of the present disclosure may be manufactured using a process comprising Scheme 5, wherein Z is halide.

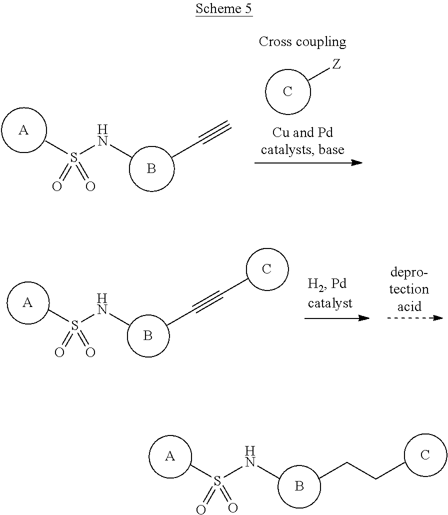

In some embodiments, compounds of the present disclosure may be manufactured using a process comprising Scheme 6, wherein Y is an amino or a functional group readily converted to amino (e.g., nitro) and R^{b} is hydrogen or methyl.

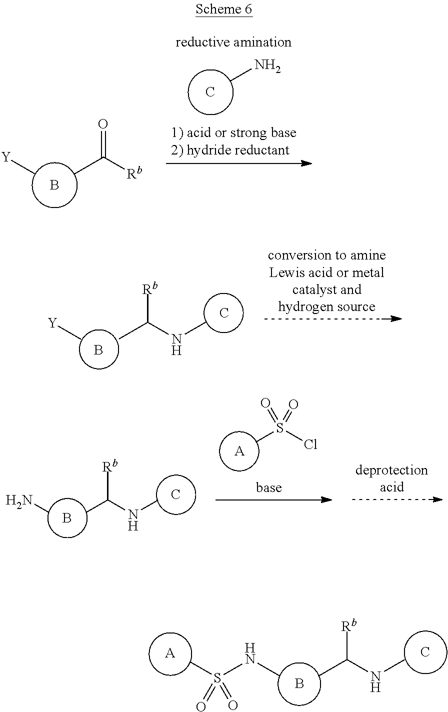

In some embodiments, compounds of the present disclosure may be synthesized using a process comprising Scheme 7, wherein Z is halide.

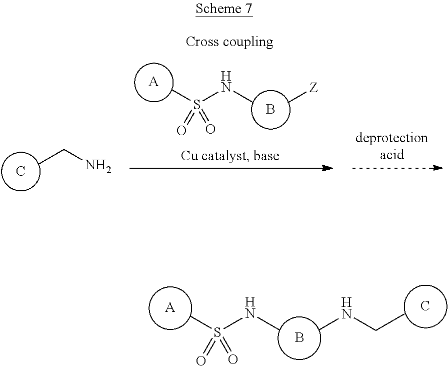

In some embodiments, compounds of the present disclosure may be synthesized using a process comprising Scheme 8, wherein Y is amino or a functional group that may be readily converted to amino (e.g., nitro).

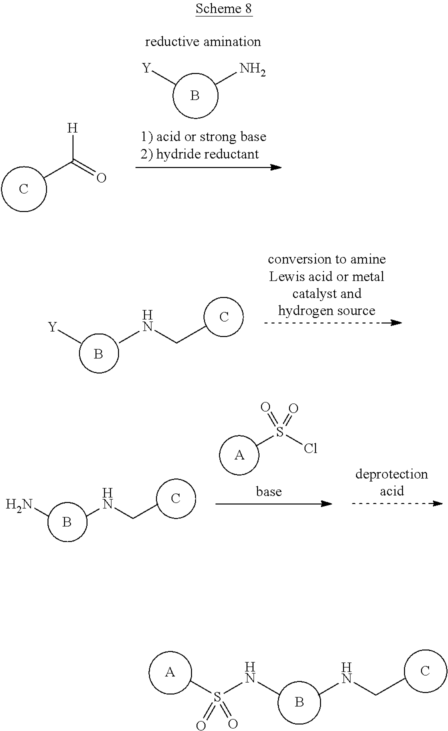

In some embodiments, compounds of the present disclosure may be synthesized using a process comprising Scheme 9, wherein Y is amino or a functional group that may be readily converted to amino (e.g., nitro).

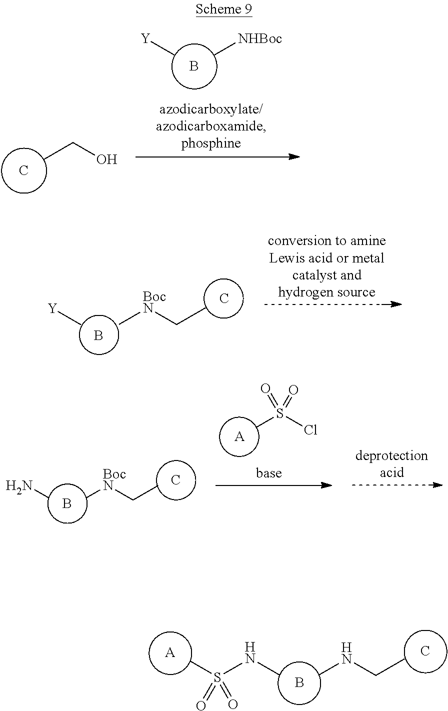

### Example 1: Synthesis of 5-fluoro-2-methoxypyridine-3-sulfonyl chloride (Intermediate

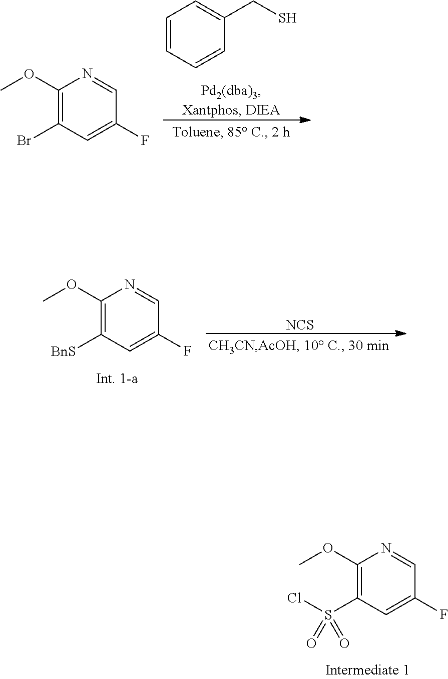

### Synthesis of Int. 1-a: 3-(benzylsulfanyl)-5-fluoro-2-methoxypyridine

Into a 2000 mL 3-necked round-bottom flask purged and maintained with an inert atmosphere of nitrogen, was placed 3-bromo-5-fluoro-2-methoxypyridine (150 g, 728 mmol, 1 equiv.), benzyl mercaptan (109 g, 878 mmol, 1.2 equiv), Pd₂(dba)₃ (41.9 g, 36 mmol, 0.05 equiv.), Xantphos (30 g, 52 mmol, 0.07 equiv), DIEA (189 g, 1.46 mol) and toluene (1.2 L). The resulting solution was stirred for 2 h at 85° C. in an oil bath, then concentrated under vacuum. The residue was applied to a silica gel column, eluting with ethyl acetate/petroleum ether (1:10) to give 3-(benzylsulfanyl)-5-fluoro-2-methoxypyridine (172 g, 95% yield) as a colorless oil.

### Synthesis of Int. 1: 5-fluoro-2-methoxypyridine-3-sulfonyl chloride

Into a 2000 mL 3-necked round-bottom flask purged and maintained with an inert atmosphere of nitrogen, was placed 3-(benzylsulfanyl)-5-fluoro-2-methoxypyridine (172 g, 690 mmol, 1 equiv.) and CH₃CN (1000 mL). This was followed by the addition of HCl (57 mL) at 10° C. To this was added NCS (368.5 g, 2760 mmol, 4 equiv) in portions at 10° C. The resulting solution was stirred for 30 min at 10˜20° C. in a water/ice bath. The resulting solution was diluted with 2000 mL of H₂O and extracted with 2×1.5 L of dichloromethane. The combined organics were washed with 2000 ml of brine and dried over anhydrous sodium sulfate, before being concentrated. The residue was applied to a silica gel column which was eluted with PE. 5-Fluoro-2-methoxypyridine-3-sulfonyl chloride (50.5 g, 32% yield) was isolated as a white solid.

LC-MS: (ES, m/z): [M+H]⁺=284.0.
¹H-NMR: (300 MHz, Chloroform-d, ppm): δ 8.37 (d, J=3.0 Hz, 1H), 8.04-8.01 (m, 1H), 4.18 (s, 3H).

### Example 2: Synthesis of 5-chloro-2-methoxypyridine-3-sulfonyl chloride (Intermediate

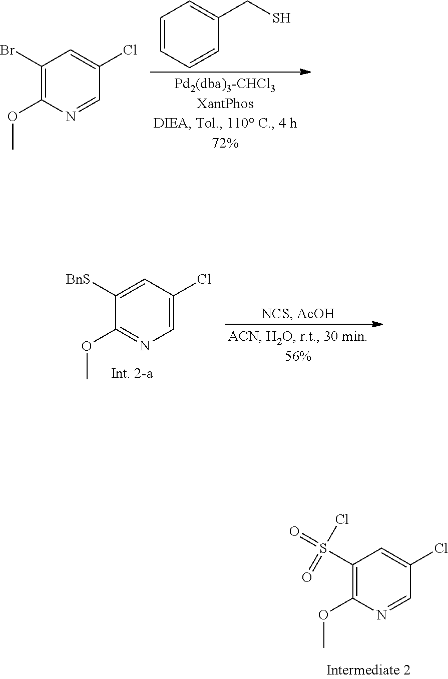

### Synthesis of Int. 2-a: 3-(benzylsulfanyl)-5-chloro-2-methoxypyridine

Into a 3000 mL round-bottom flask purged and maintained with an inert atmosphere of nitrogen, was placed 3-bromo-5-chloro-2-methoxypyridine (150 g, 678 mmol, 1.0 equiv), toluene (1500 mL), phenylmethanethiol (92.6 g, 746 mmol, 1.1 equiv), DIEA (175.4 g, 1357 mmol, 2.0 equiv), XantPhos (3.92 g, 6 mmol, 0.01 equiv) and Pd₂(dba)₃-CHCl₃ (5.27 g, 5 mmol 0.0075 equiv). The resulting solution was stirred for 4 h at 110° C. in an oil bath. The solids were removed by filtration and the filtrate concentrated. The residue was applied to a silica gel column, eluting with PE:EA=20:1. Concentration of the appropriate fractions gave a solid which was slurried with PE (3 V). The solid was removed by filtration and dried to give 3-(benzylsulfanyl)-5-chloro-2-methoxypyridine (130 g, 72% yield) as a yellow solid.

### Synthesis of Int. 2: 5-chloro-2-methoxypyridine-3-sulfonyl chloride

Into a 4000 mL round-bottom flask, was placed 3-(benzylsulfanyl)-5-chloro-2-methoxypyridine (130 g, 490 mmol, 1 equiv), MeCN (2600 mL, 20 V), H₂O (130 mL, 1 V), acetic acid (294 g, 491 mmol, 10 equiv) and NCS (196 g, 1470 mmol, 3.0 equiv). The resulting solution was stirred for 30 min at 25° C. The reaction was quenched by the addition 1000 mL of water and extracted with 2×1000 mL of ethyl acetate. The combined organics were washed with H₂O and concentrated. The resulting solution was diluted with 500 mL of diethyl ether and the solids removed by filtration. The filtrate was concentrated, and the residue purified by silica gel column chromatography, eluting with PE/THF (100:1). The concentrated product was slurried with 300 mL hexane and kept at 0° C. for 1 h. The solids were removed by filtration and dried to give 5-chloro-2-methoxypyridine-3-sulfonyl chloride (66.5 g, 56% yield) as a white solid.

LCMS: (ES, m/z): 313 [M+1]⁺
¹H-NMR (300 MHz, CDCl₃, ppm): δ 8.45 (d, J=2.5 Hz, 1H), 8.23 (d, J=2.6 Hz, 1H), 4.21 (s, 3H).

### Example 3: Synthesis of 5-cyano-2-methoxypyridine-3-sulfonyl chloride (Intermediate

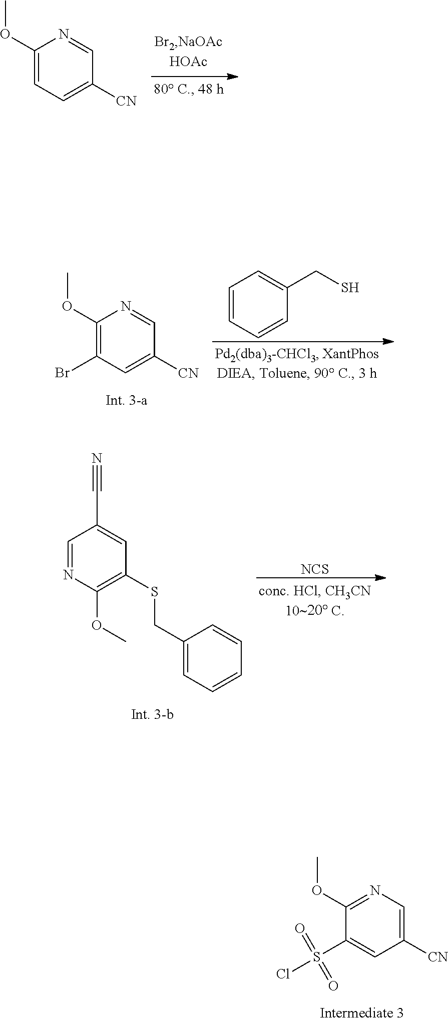

### Synthesis of Int. 3-a: 5-bromo-6-methoxypyridine-3-carbonitrile

Into a 2 L round-bottom flask were added 6-methoxypyridine-3-carbonitrile (100 g, 746 mmol, 1 equiv), HOAc (1000 mL), NaOAc (61.1 g, 746 mmol, 1 equiv) and Br₂ (235 g, 1490 mmol, 2 equiv) at room temperature. The resulting mixture was stirred for 48 h at 80° C. The mixture was allowed to cool to room temperature and was diluted with ice water (3 L). The precipitated solids were collected by filtration and suspended in 1 L of PE:EA=5:1 which was stirred for 1 h at room temperature. The precipitated solids were collected by filtration and dried to give 5-bromo-6-methoxypyridine-3-carbonitrile (70 g, 44% yield) as a light yellow solid.

### Synthesis of Int. 3-b: 5-(benzylsulfanyl)-6-methoxypyridine-3-carbonitrile

Into a 3 L 3-necked round-bottom flask, were added 5-bromo-6-methoxypyridine-3-carbonitrile (70 g, 330 mmol, 1 equiv), toluene (1400 mL) and benzyl mercaptan (43 g, 347 mmol, 1.05 equiv) at room temperature. To this was added Pd₂(dba)₃·CHCl₃ (17 g, 16.5 mmol, 0.05 equiv), Xantphos (19 g, 33 mmol, 0.1 equiv) and DIEA (128 g, 990 mmol, 3 equiv) under nitrogen atmosphere. The resulting mixture was stirred for 3 h at 90° C., then was cooled and filtered. The filtrate was concentrated under reduced pressure, and the residue purified by silica gel column chromatography, eluting with PE/THF (10:1). 5-(Benzylsulfanyl)-6-methoxypyridine-3-carbonitrile (65 g, 77% yield) was obtained as a light brown solid.

### Synthesis of Int. 3: 5-cyano-2-methoxypyridine-3-sulfonyl chloride

Into a 2 L 3-necked round-bottom flask were added 5-(benzylsulfanyl)-6-methoxypyridine-3-carbonitrile (65 g, 2540 mmol, 1 equiv), MeCN (520 g), H₂O (260 g) and HCl (21 mL, 254 mmol, 1 equiv) at room temperature. To this was added NCS (101 g, 762 mmol, 3 equiv) in portions at 0° C. The resulting mixture was stirred for 0.5 h at room temperature, then was cooled to 0° C. and stirred for 1 h. The precipitated solids were collected by filtration and dried to afford 5-cyano-2-methoxypyridine-3-sulfonyl chloride (25 g, 42% yield) as a white solid.

LC-MS: (ES, m/z): 302 [M−1]⁻
¹H-NMR: (300 MHz, CDCl3, ppm): δ 8.78 (d, J=2.2 Hz, 1H), 8.51 (d, J=2.2 Hz, 1H), 4.31 (s, 3H).

### Example 4: Synthesis of 5-fluoro-2-methylpyridine-3-sulfonyl chloride (Intermediate 4)

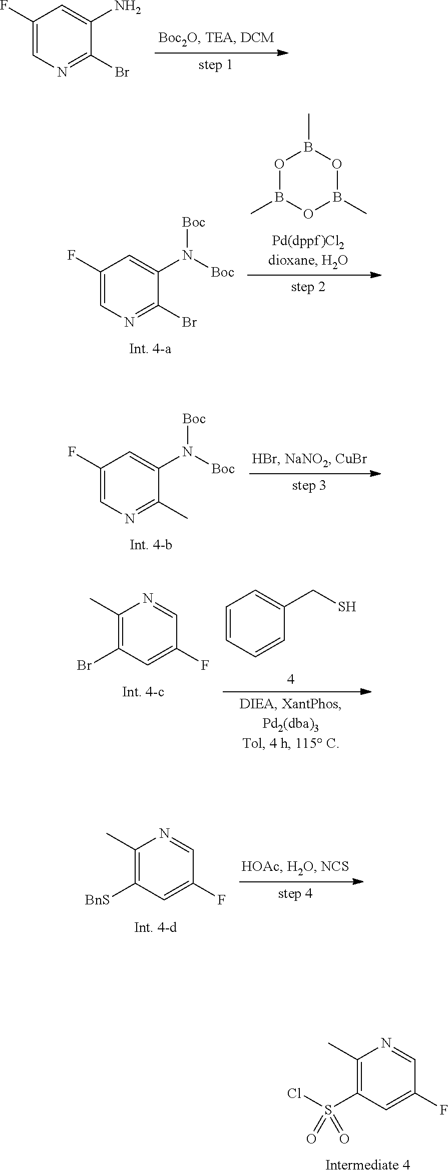

### Synthesis of Int. 4-a: tert-butyl N-(2-bromo-5-fluoropyridin-3-yl)-N-(tert-butoxycarbonyl)carbamate

Into a 500 mL 3-necked round-bottom flask was placed 2-bromo-5-fluoropyridin-3-amine (20 g, 1 equiv), DCM (220 mL) and TEA (44 mL, 3 equiv). This was followed by the addition of Boc₂O (57 g, 2.5 equiv) in several batches. The resulting solution was stirred for 14 h at room temperature. The reaction mixture was concentrated, and the residue was applied to a silica gel column, eluting with ethyl acetate/petroleum ether (1:5). This resulted in tert-butyl N-(2-bromo-5-fluoropyridin-3-yl)-N-(tert-butoxycarbonyl)carbamate (33 g) as a white solid.

### Synthesis of Int. 4-b: tert-butyl N-(tert-butoxycarbonyl)-N-(5-fluoro-2-methylpyridin-3-yl)carbamate

Into a 500 mL 3-necked round-bottom flask purged and maintained with an inert atmosphere of nitrogen was placed tert-butyl N-(2-bromo-5-fluoropyridin-3-yl)-N-(tert-butoxycarbonyl)carbamate (33 g, 84 mmol, 1 equiv), 1,4-dioxane (150 mL), trimethyl-1,3,5,2,4,6-trioxatriborinane (21.2 g, 169 mmol, 2 equiv), K₂CO₃ (35 g, 253 mmol, 3 equiv) and Pd(dppf)Cl₂ (3.09 g, 4.2 mmol, 0.05 equiv). The resulting solution was stirred overnight at 110° C. under N₂ atmosphere in an oil bath. The reaction mixture was cooled to room temperature and filtered. The filtrate was diluted with 200 mL of H₂O and extracted with 3×100 mL of ethyl acetate. The combined organics were concentrated, and the residue applied to a silica gel column, eluting with ethyl acetate/petroleum ether (1:10-1:5). Tert-butyl N-(tert-butoxycarbonyl)-N-(5-fluoro-2-methylpyridin-3-yl)carbamate (16 g, 58% yield) was isolated as a yellow solid.

### Synthesis of Int. 4-c: 3-bromo-5-fluoro-2-methylpyridine

Into a 2 L 3-necked round-bottom flask, was placed tert-butyl N-(tert-butoxycarbonyl)-N-(5-fluoro-2-methylpyridin-3-yl)carbamate (50 g, 153 mmol, 1 equiv) and HBr (1 L, 48%). This was followed by the addition of a solution of NaNO₂ (11.6 g, 169 mmol, 1.1 equiv) in H₂O (100 mL) dropwise with stirring at 0-5° C. The resulting solution was stirred for 30 min in an ice bath. To this was added CuBr (24.2 g, 169 mmol, 1.1 equiv) at 0° C. The resulting solution was stirred for 1 h at room temperature, then quenched by the addition of 1 L water/ice. The pH of the solution was adjusted to 8 with Na₂CO₃ and the resulting mixture was extracted with 3×200 mL of ethyl acetate. The combined organics were dried over anhydrous sodium sulfate and concentrated. The residue was applied to a silica gel column, eluting with ethyl acetate/petroleum ether (1:50). 3-Bromo-5-fluoro-2-methylpyridine (13 g, 45% yield) was isolated as a white solid.

### Synthesis of Int. 4-d: 3-(benzylsulfanyl)-5-fluoro-2-methylpyridine

Into a 250 mL 3-necked round-bottom flask purged and maintained with an inert atmosphere of nitrogen was placed 3-bromo-5-fluoro-2-methylpyridine (13 g, 68 mmol, 1 equiv), toluene (130 mL), benzyl mercaptan (12.8 g, 103 mmol, 1.5 equiv), DIEA (17.7 g, 137 mmol, 2 equiv), XantPhos (3.96 g, 6.8 mmol, 0.1 equiv) and Pd₂(dba)₃ (3.13 g, 3.4 mmol, 0.05 equiv). The resulting solution was stirred for 4 h at 115° C., then cooled and concentrated. The residue was applied to a silica gel column, eluting with ethyl acetate/petroleum ether (1:20) to give 3-(benzylsulfanyl)-5-fluoro-2-methylpyridine (11 g, 69% yield) as a yellow solid.

### Synthesis of Intermediate 4: 5-fluoro-2-methylpyridine-3-sulfonyl chloride

Into a 2000 mL 3-necked round-bottom flask, was placed 3-(benzylsulfanyl)-5-fluoro-2-methylpyridine (45 g, 193 mmol, 1 equiv), HOAc (700 mL) and H₂O (200 mL). This was followed by the addition of NCS (103 g, 772 mmol, 4 equiv), the temperature being maintained under 20° C. The resulting solution was stirred for 2 h at room temperature. The reaction was quenched by the addition of 700 mL of water, and the resulting solution was extracted with 3×300 mL of dichloromethane. The combined organics were concentrated, and the residue applied to a silica gel column, eluting with ethyl acetate/petroleum ether (1:10). 5-Fluoro-2-methylpyridine-3-sulfonyl chloride (25.4 g, 63% yield) was obtained as a yellow oil.

LCMS: (ES, m/z): 210 [M+1]⁺
¹H-NMR: (300 MHz, CDCl₃, ppm): δ 8.72-8.71 (d, J=3.0 Hz, 1H), 8.11-8.08 (dd, J=3.0 Hz, 1H), 3.01 (s, 3H).

### Example 5: Synthesis of 5-chloro-N-[3,5-difluoro-4-[1-(1H-imidazol-2-yl)imidazo[1,5-a]pyridin-6-yl]pyridin-2-yl]-2-methoxypyridine-3-sulfonamide (Intermediate 5)

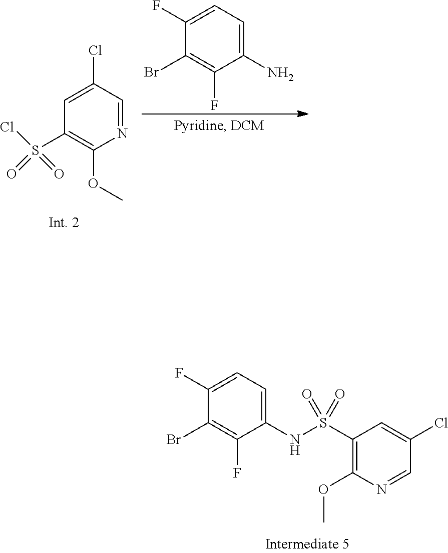

### Synthesis of Intermediate 5: N-(3-bromo-2,4-difluorophenyl)-5-chloro-2-methoxypyridine-3-sulfonamide

To a solution of 3-bromo-2,4-difluoroaniline (5 g, 24 mmol, 1 eq) in DCM (100 mL) were added 5-chloro-2-methoxypyridine-3-sulfonyl chloride (8.73 g, 36 mmol, 1.5 eq) and pyridine (5.7 g, 72 mmol, 3 eq). The resulting solution was stirred for 1 hour at room temperature. The reaction was concentrated and purified by column chromatography over silica gel (eluent: PE:EA=8:1) to afford N-(3-bromo-2,4-difluorophenyl)-5-chloro-2-methoxypyridine-3-sulfonamide (7 g, 70% yield) as a white solid.

LCMS (ES, m/z): [M+H]⁺: 412
¹H NMR (300 MHz, Chloroform-d) δ 8.30 (d, J=2.6 Hz, 1H), 8.05 (d, J=2.6 Hz, 1H), 7.56 (td, J=8.9, 5.4 Hz, 1H), 7.28 (d, J=3.4 Hz, 1H), 6.96 (ddd, J=9.4, 7.6, 2.1 Hz, 1H), 4.16 (s, 3H).

### Example 6: Synthesis of 3-ethynyl-2,4-difluoroaniline & 5-chloro-N-(3-ethynyl-2,4-difluorophenyl)-2-methoxypyridine-3-sulfonamide (Intermediate 6 & Intermediate 7)

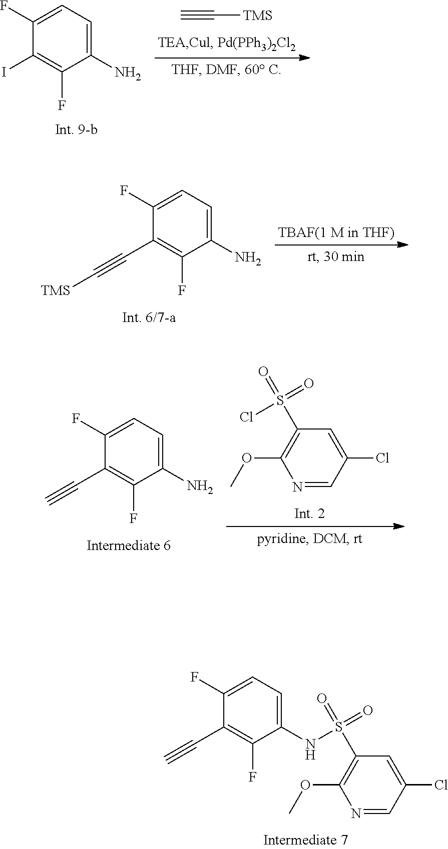

### Synthesis of Int. 6/7-a: 2,4-difluoro-3-[2-(trimethylsilyl)ethynyl]aniline

Into a 100 mL round-bottom flask purged and maintained with an inert atmosphere of nitrogen, was placed 2,4-difluoro-3-iodoaniline (1.8 g, 6.9 mmol, 1 equiv), THF (14 mL), DMF (14 mL), TEA (2.1 g, 20.6 mmol, 3 equiv), CuI (0.1 g, 0.7 mmol, 0.1 equiv), Pd(PPh₃)₂Cl₂ (0.5 g, 0.7 mmol, 0.1 equiv) and trimethylsilylacetylene (2.0 g, 20.6 mmol, 3 equiv). The resulting solution was stirred overnight at 60° C. in an oil bath. The reaction mixture was cooled and diluted with 50 mL of water. The resulting solution was extracted with 2×50 mL of ethyl acetate and the organic layers combined. This was washed with 3×50 ml of water, dried over anhydrous sodium sulfate and concentrated. The residue was applied onto a silica gel column, eluting with ethyl acetate/petroleum ether (1:10). The appropriate fractions were combined and concentrated to give 2,4-difluoro-3-[2-(trimethylsilyl)ethynyl]aniline (2 g, 97% yield) as a brown oil.

LCMS (ES, m/z): [M+H]⁺: 226

### Synthesis of Int. 6: 3-ethynyl-2,4-difluoroaniline

Into a 50 mL round-bottom flask, was placed 2,4-difluoro-3-[2-(trimethylsilyl)ethynyl]aniline (1.7 g, 7.5 mmol, 1 equiv). TBAF (15 mL, 1 M in THF) was added and the solution was stirred for 30 min at 25° C. The resulting mixture was concentrated, and the residue applied to a silica gel column, eluting with ethyl acetate/petroleum ether (1:5). The collected fractions were combined and concentrated to give 3-ethynyl-2,4-difluoroaniline (1.05 g, 91% yield) as a brown solid.

LCMS (ES, m/z): [M+H]⁺: 154

### Synthesis of Int. 7: 5-chloro-N-(3-ethynyl-2,4-difluorophenyl)-2-methoxypyridine-3-sulfonamide

Into an 8 mL vial was placed 3-ethynyl-2,4-difluoroaniline (300 mg, 2 mmol, 1 equiv), DCM (10 mL), pyridine (1.5 g, 19.6 mmol, 10 equiv) and 5-chloro-2-methoxypyridine-3-sulfonyl chloride (569 mg, 2.4 mmol, 1.2 equiv). The resulting solution was stirred for 1 h then concentrated. The crude product was purified by Flash-Prep-HPLC with the following conditions: Column, WelFlash TM C18-I, Spherical C18 20-40 m, 120 g; mobile phase: 5-95% MeCN/0.1% aqueous formic acid; Detector, 220 nm; to give 5-chloro-N-(3-ethynyl-2,4-difluorophenyl)-2-methoxypyridine-3-sulfonamide (560 mg, 80% yield) as a brown solid.

LCMS (ES, m/z): [M+H]⁺: 359

### Example 7: Synthesis of 2,6-difluoro-3-nitrobenzaldehyde (Intermediate 8)

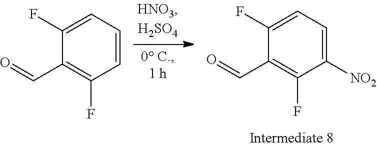 [SMILES: O=Cc1c(F)cccc1F ; O=Cc1c(F)ccc([N+](=O)[O-])c1F]

### Example 7: Synthesis of 2,6-difluoro-3-nitrobenzaldehyde (Intermediate 8)

To a stirred solution of 2,6-difluorobenzaldehyde (1 g, 7 mmol, 1 equiv) in H₂SO₄ (2.5 mL) was added HNO₃ (1 mL, 68%) dropwise at 0° C. under air atmosphere. The resulting mixture was stirred for 1 h at 0° C. The reaction was quenched with water/ice at 0° C., and filtered. The filter cake was washed with water (2×10 mL), and the resulting solid was dried under infrared light to afford crude 2,6-difluoro-3-nitrobenzaldehyde (1 g, 76% yield) as a yellow solid which was used in next step directly without further purification.

### Example 8: 2,4-difluoro-3-(2-[1H-pyrazolo[3,4-b]pyridin-5-yl]ethyl)aniline (Intermediate 9)

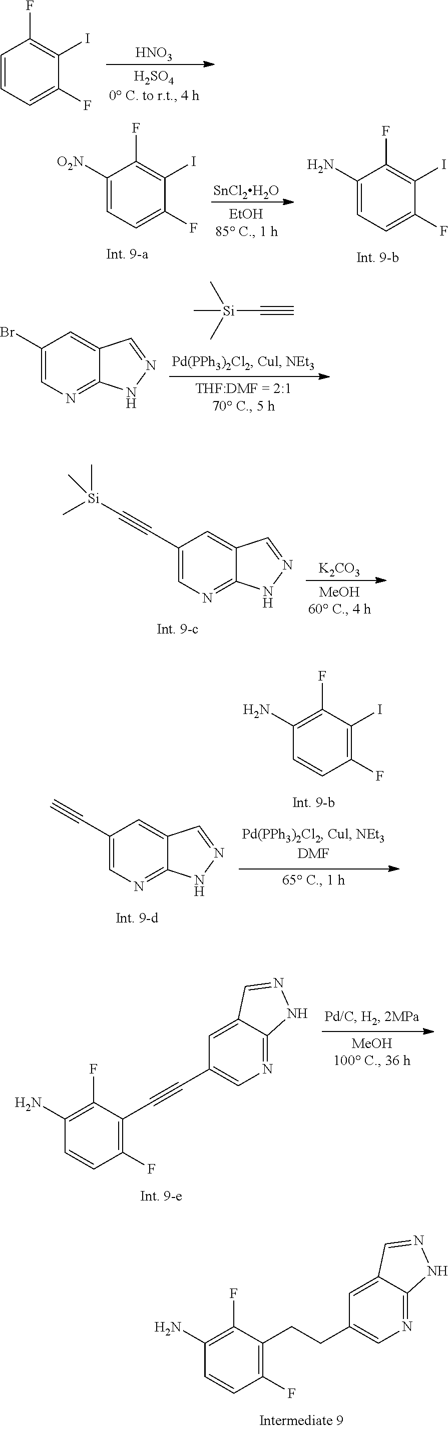

### Synthesis of Int. 9-a: 1,3-difluoro-2-iodo-4-nitrobenzene

To a stirred suspension of 1,3-difluoro-2-iodobenzene (25 g, 100 mmol, 1 equiv) in H₂SO₄ (100 mL) was added fuming HNO₃ (16.4 g, 260 mmol, 2.5 equiv) dropwise at 0° C. The mixture was stirred for 4 h at room temperature. The reaction mixture was poured into ice water (1 L) and the resulting mixture extracted with EA (3×500 mL). The combined organic layers were washed with saturated aqueous NaHCO₃ (2×200 mL), brine (2×200 mL), dried over anhydrous Na₂SO₄ and filtered. The filtrate was concentrated under reduced pressure to afford 1,3-difluoro-2-iodo-4-nitrobenzene (28.3 g, 95% yield) as a yellow solid which was used directly in next step without further purification.

### Synthesis of Int. 9-b: 2,4-difluoro-3-iodoaniline

To a stirred suspension of 1,3-difluoro-2-iodo-4-nitrobenzene (26 g, 92 mmol, 1 equiv) in EtOH (300 mL) was added SnCl₂·2H₂O (62 g, 280 mmol, 3 equiv) portion wise at room temperature. The resulting solution was stirred for 1 h at 85° C., cooled to room temperature and concentrated under reduced pressure. The residue was basified to pH >9 with saturated aqueous NaOH and the resulting mixture extracted with EtOAc (3×500 mL). The combined organic extracts were washed with brine (2×200 mL), dried over anhydrous Na₂SO₄, filtered and concentrated under reduced pressure. The residue was purified by silica gel column chromatography, eluting with PE:EtOAc (9:1 to 4:1) to afford 2,4-difluoro-3-iodoaniline (19 g, 80% yield) as a black solid.

### Synthesis of Int. 9-c: 5-[2-(trimethylsilyl)ethynyl]-1H-pyrazolo[3,4-b]pyridine

To a mixture of 5-bromo-1H-pyrazolo[3,4-b]pyridine (7.6 g, 38 mmol, 1 equiv) and trimethylsilylacetylene (11.3 g, 115 mmol, 3 equiv) in THE (180 mL) and DMF (90 mL) under a nitrogen atmosphere was added CuI (730 mg, 3.8 mmol, 0.1 equiv), Pd(PPh₃)₂Cl₂ (2.7 g, 3.8 mmol, 0.1 equiv) and TEA (38.6 g, 382 mmol, 10 equiv). The resulting mixture was stirred for 5 h at 70° C. under nitrogen atmosphere. The mixture was allowed to cool to room temperature, then poured onto the ice water (500 mL) and the resulting mixture diluted with EtOAc (500 mL). The aqueous layer was extracted with EtOAc (3×100 mL) and the combined organic extracts were washed with water (3×200 mL) and brine (1×100 mL), dried over anhydrous Na₂SO₄ and concentrated under reduced pressure. The residue was purified by silica gel column chromatography, eluting with PE/EtOAc (20:1 to 1:2) to afford 5-[2-(trimethylsilyl)ethynyl]-1H-pyrazolo[3,4-b]pyridine (8 g, 97% yield) as a light yellow solid.

LCMS (ES, m/z): [M+H]⁺: 216

### Synthesis of Int. 9-d: 5-ethynyl-1H-pyrazolo[3,4-b]pyridine

To a stirred solution of 5-[2-(trimethylsilyl)ethynyl]-1H-pyrazolo[3,4-b]pyridine (8.0 g, 37 mmol, 1 equiv) in MeOH (190 mL) was added K₂CO₃ (10 g, 74 mmol, 2 equiv) in portions under a nitrogen atmosphere. The resulting mixture was stirred for 4 h at 60° C. and then allowed to cool to room temperature. The resulting mixture was filtered and the filter cake washed with MeOH (3×100 mL). The filtrate was concentrated under reduced pressure. The residue was purified by silica gel column chromatography, eluting with PE/EtOAc (5:1 to 0:1) to afford 5-ethynyl-1H-pyrazolo[3,4-b]pyridine (4.1 g, 77% yield) as a light yellow solid.

LCMS (ES, m/z): [M+H]⁺: 144

### Synthesis of Int. 9-e: 2,4-difluoro-3-(2-[1H-pyrazolo[3,4-b]pyridin-5-yl]ethynyl)aniline

Into a 250 mL round-bottom flask were added 5-ethynyl-1H-pyrazolo[3,4-b]pyridine (1.5 g, 11 mmol, 1.2 equiv), 2,4-difluoro-3-iodoaniline (2.3 g, 8.8 mmol, 1 equiv), CuI (170 mg, 0.9 mmol, 0.1 equiv) and Pd(PPh₃)₂Cl₂ (620 mg, 0.9 mmol, 0.1 equiv), followed by DMF (80 mL) at room temperature. The mixture was sparged with N₂ for 10 min and then TEA (2.7 g, 27 mmol, 3 equiv) was added. The resulting mixture was stirred for 1 h at 65° C. and then allowed to cool to room temperature. The reaction was poured into the ice water (200 mL) and diluted with EtOAc (200 mL). The aqueous layer was extracted with EtOAc (3×100 mL) and the combined organic layers were washed with water (3×100 mL), dried over anhydrous Na₂SO₄ and concentrated under reduced pressure. The residue was purified by silica gel column chromatography, eluting with PE/EtOAc (5:1 to 1:1) to afford 2,4-difluoro-3-(2-[1H-pyrazolo[3,4-b]pyridin-5-yl]ethynyl)aniline (1.7 g, 71% yield) as a light yellow solid.

LCMS (ES, m/z): [M+H]⁺: 271

### Synthesis of Intermediate 9: 2,4-difluoro-3-(2-[1H-pyrazolo[3,4-b]pyridin-5-yl]ethyl)aniline

To a stirred solution of 2,4-difluoro-3-(2-[1H-pyrazolo[3,4-b]pyridin-5-yl]ethynyl)aniline (1.1 g, 4 mmol, 1 equiv) in MeOH (180 mL) was added Pd/C (220 mg). The mixture was stirred at 100° C. for 36 h under a hydrogen atmosphere (2 MPa). The mixture was allowed to cool down to 30° C. and the resulting mixture filtered. The filter cake was washed with hot MeOH (3×150 mL) and the filtrate concentrated under reduced pressure to afford 2,4-difluoro-3-(2-[1H-pyrazolo[3,4-b]pyridin-5-yl]ethyl)aniline (600 mg, 54% yield).

LCMS (ES, m/z): [M+H]⁺: 275

### Example 9: Synthesis of 2,4-difluoro-3-(4,4,5,5-tetramethyl-1,3,2-dioxaborolan-2-yl)aniline (Intermediate 10)

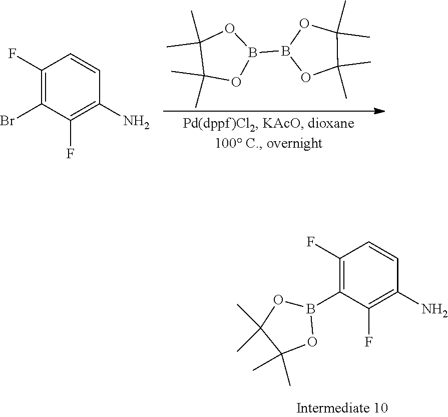

### Synthesis of Intermediate 10: 2,4-difluoro-3-(4,4,5,5-tetramethyl-1,3,2-dioxaborolan-2-yl)aniline

3-Bromo-2,4-difluoroaniline (10 g, 48 mmol, 1 equiv), Pd(dppf)Cl₂ (3.5 g, 4.8 mmol, 0.1 equiv), bis(pinacolato)diboron (18.3 g, 72 mmol, 1.5 equiv) and KOAc (14.2 g, 144.2 mmol, 3 equiv) were dissolved in dioxane (240 mL). The resulting solution was stirred overnight at 100° C. in an oil bath. The reaction mixture was cooled and the solids removed by filtration. The filtrate was concentrated and diluted with DCM (100 mL), then washed with 2×100 mL of water and 1×100 mL of brine. The organic phase was dried over anhydrous sodium sulfate and concentrated under reduced pressure. The residue was applied to a silica gel column, eluting with ethyl acetate/petroleum ether (1/10). 2,4-Difluoro-3-(4,4,5,5-tetramethyl-1,3,2-dioxaborolan-2-yl)aniline (8 g, 65% yield) was isolated as a yellow solid.

LCMS (ES, m/z): [M+H]⁺: 256

### Example 10: Synthesis of 3,5-difluoro-4-iodopyridin-2-amine (Intermediate 11)

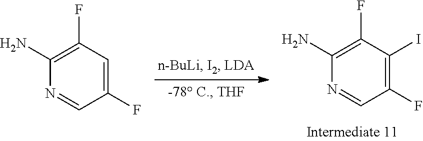 [SMILES: Nc1ncc(F)cc1F ; Nc1ncc(F)c(I)c1F]

### Synthesis of Intermediate 11: 3,5-difluoro-4-iodopyridin-2-amine

To a stirred solution of 3,5-difluoropyridin-2-amine (5 g, 38 mmol, 1 equiv) in THE (200 mL) was added LDA (61 mL, 123 mmol, 3 equiv) dropwise at −78° C. under N₂ atmosphere. The solution was stirred for 1.5 h at −78° C. To the resulting mixture was added a solution of I₂ (34 g, 135 mmol, 3.5 equiv) in THE (50 mL) dropwise at −78° C. This mixture was stirred for additional 0.5 h at −78° C., then was quenched with saturated aqueous Na₂S₂O₃ (100 mL). The reaction was extracted with EA (3×50 mL), and the combined organics were washed with brine (2×50 mL) and dried over anhydrous sodium sulfate before being concentrated. The residue was purified by silica gel column chromatography, eluting with PE:EA (3:1) to afford 3,5-difluoro-4-iodopyridin-2-amine (7.8 g, 79% yield) as a yellow solid.

LCMS (ES, m/z): [M+H]⁺: 257

### Example 11: Synthesis of 4-chloro-3-fluoropicolinaldehyde (Intermediate 12)

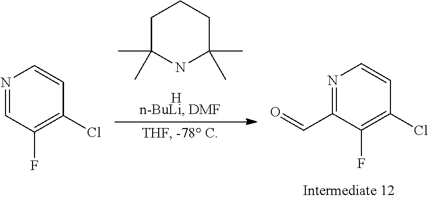

### Synthesis of Intermediate 12: 4-chloro-3-fluoropicolinaldehyde

To a stirred solution of n-BuLi (38 mL, 92 mmol, 1.2 equiv) in THF (100 mL) was added 2,2,6,6-tetramethylpiperidine (13 g, 92 mmol, 1.2 equiv) dropwise at −78° C. under nitrogen atmosphere. The resulting mixture was stirred for 10 min at −78° C., then 4-chloro-3-fluoropyridine (10 g, 76 mmol, 1 equiv) was added dropwise over 10 min. The resulting mixture was stirred for additional 0.5 h at −78° C., before DMF (6.1 g, 84 mmol, 1.1 equiv) was added dropwise over 10 min. The resulting mixture was stirred for 0.5 h at −78° C. then quenched by the addition of saturated aqueous NaHCO₃ solution (50 mL). The resulting mixture was extracted with EA (3×100 mL) and the combined organics were washed with brine (1×100 mL), then dried over anhydrous sodium sulfate. The solution was concentrated under reduced pressure to give crude 4-chloro-3-fluoropicolinaldehyde (10 g) as a yellow oil, which was used in the next step directly without further purification.

LCMS (ES, m/z): [M+H]⁺: 160

### Example 12: Synthesis of 5-chloro-N-[2,4-difluoro-3-([1H-pyrazolo[3,4-b]pyridin-5-yloxy]methyl)phenyl]-2-methoxypyridine-3-sulfonamide (Compound 1)

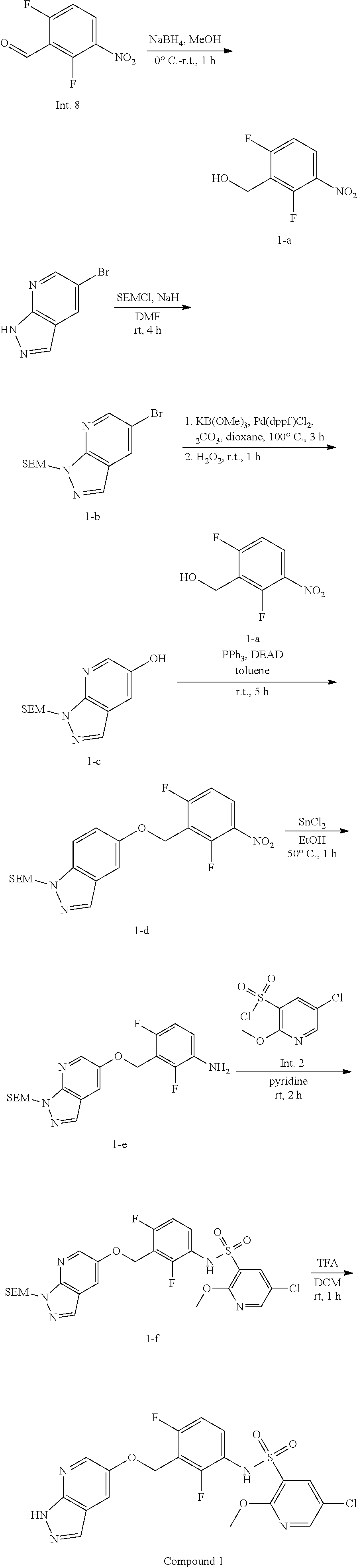

### Synthesis of 1-a: (2,6-difluoro-3-nitrophenyl)methanol

To a stirred solution of 2,6-difluoro-3-nitrobenzaldehyde (1 g, 5.3 mmol, 1 equiv) in MeOH (10 mL) was added NaBH₄ (0.4 g, 10.6 mmol, 2 equiv) in portions at 0° C. under nitrogen atmosphere and the mixture stirred for 1 h. The resulting mixture was concentrated under vacuum, and the residue was purified by silica gel column chromatography, eluting with PE/EtOAc (5:1) to afford (2,6-difluoro-3-nitrophenyl)methanol (720 mg, 71% yield) as a yellow oil.

### Synthesis of 1-b: 5-bromo-1-[[2-(trimethylsilyl)ethoxy]methyl]pyrazolo[3,4-b]pyridine

To a stirred solution of 5-bromo-1H-pyrazolo[3,4-b]pyridine (1 g, 5 mmol, 1 equiv) in DMF (15 mL) was added NaH (157 mg, 6.5 mmol, 1.3 equiv, 60% in oil) in portions at 0° C. under nitrogen atmosphere. The reaction was stirred for 15 min then SEMCl (1 g, 6 mmol, 1.2 equiv) was added dropwise at 0° C. The resulting mixture was stirred for additional 4 h at room temperature. The reaction was quenched with water at room temperature and the aqueous layer extracted with EtOAc (2×10 mL). The combined organic layers were washed with brine (2×10 mL) and concentrated under vacuum. The residue was purified by silica gel column chromatography, eluting with PE/EtOAc (5:1) to afford 5-bromo-1-[[2-(trimethylsilyl)ethoxy]methyl]pyrazolo[3,4-b]pyridine (1.2 g, 72% yield) as a yellow solid.

LCMS (ES, m/z): [M+H]⁺: 328

### Synthesis of 1-c: 1-[[2-(trimethylsilyl)ethoxy]methyl]pyrazolo[3,4-b]pyridin-5-ol

To a stirred mixture of 5-bromo-1-[[2-(trimethylsilyl)ethoxy]methyl]pyrazolo[3,4-b]pyridine (1 g, 3 mmol, 1 equiv) and B(OMe)₃ (633 mg, 6 mmol, 2 equiv) in dioxane (20 mL) were added K₂CO₃ (1 g, 7.6 mmol, 2.5 equiv) and Pd(dppf)Cl₂ (223 mg, 0.3 mmol, 0.1 equiv) in portions at room temperature under nitrogen atmosphere. The resulting mixture was stirred at 100° C. for 3 h, then was allowed to cool to room temperature. To this was added H₂O₂ (30%) (207 mg, 6 mmol, 2 equiv) dropwise over 5 min at room temperature. The resulting mixture was stirred for additional 1 h, then concentrated under vacuum. The residue was purified by silica gel column chromatography, eluting with hexane/EtOAc (1:1) to afford 1-[[2-(trimethylsilyl)ethoxy]methyl]pyrazolo[3,4-b]pyridin-5-ol (0.9 g, 92% yield) as an off-white solid.

LCMS (ES, m/z): [M+H]⁺: 266

### Synthesis of 1-d: 5-[(2,6-difluoro-3-nitrophenyl)methoxy]-1-[[2-(trimethylsilyl)ethoxy]methyl]pyrazolo[3,4-b]pyridine

To a stirred mixture of (2,6-difluoro-3-nitrophenyl)methanol (1.2 g, 6.4 mmol, 2 equiv) and 1-[[2-(trimethylsilyl)ethoxy]methyl]pyrazolo[3,4-b]pyridin-5-ol (860 mg, 3.2 mmol, 1 equiv) in toluene (50 mL) was added PPh₃ (2.1 g, 8 mmol, 2.5 equiv) in portions at room temperature under nitrogen atmosphere. The resulting mixture was stirred for 20 min, then DEAD (1.7 g, 9.6 mmol, 3 equiv) was added dropwise over 5 min. The resulting mixture was stirred for 5 h at room temperature, then diluted with water (50 mL). This was extracted with EtOAc (2×30 mL). The combined organic layers were washed with brine (2×20 mL) and dried over anhydrous Na₂SO₄. After filtration, the filtrate was concentrated under reduced pressure and the residue was purified by silica gel column chromatography, eluting with PE/EtOAc (1:3) to afford 5-[(2,6-difluoro-3-nitrophenyl)methoxy]-1-[[2-(trimethylsilyl)ethoxy]methyl]pyrazolo[3,4-b]pyridine (560 mg, 31% yield) as a yellow solid.

LCMS (ES, m/z): [M+H]⁺: 437

### Synthesis of 1-e: 2,4-difluoro-3-[[(1-[[2-(trimethylsilylethoxy]methyl]pyrazolo[3,4-b]pyridin-5-yl)oxy]methyl]aniline

To a stirred solution of 5-[(2,6-difluoro-3-nitrophenyl)methoxy]-1-[[2-(trimethylsilyl)ethoxy]methyl]pyrazolo[3,4-b]pyridine (540 mg, 1.2 mmol, 1 equiv) in EtOH (10 mL) was added SnCl₂ (469 mg, 2.4 mmol, 2 equiv) in portions at room temperature under nitrogen atmosphere. The resulting mixture was stirred for 1 h at 50° C., then was concentrated under vacuum. The residue was purified by silica gel column chromatography, eluting with PE/EtOAc (1:1) to afford 2,4-difluoro-3-[[(1-[[2-(trimethylsilyl)ethoxy]methyl]pyrazolo[3,4-b]pyridin-5-yl)oxy]methyl]aniline (280 mg, 55% yield) as a yellow solid.

LCMS (ES, m/z): [M+H]⁺: 407

### Synthesis of 1-f: 5-chloro-N-(2,4-difluoro-3-(((1-((2-(trimethylsilyl)ethoxy)methyl)-1H-pyrazolo[3,4-b]pyridin-5-yl)oxy)methyl)phenyl)-2-methoxypyridine-3-sulfonamide

To a stirred solution of 2,4-difluoro-3-[[(1-[[2-(trimethylsilyl)ethoxy]methyl]pyrazolo[3,4-b]pyridin-5-yl)oxy]methyl]aniline (260 mg, 0.64 mmol, 1 equiv) in pyridine (5 mL) was added 5-chloro-2-methoxypyridine-3-sulfonyl chloride (232 mg, 0.96 mmol, 1.5 equiv) dropwise at room temperature under nitrogen atmosphere. The resulting mixture was stirred for 2 h, then concentrated under vacuum. The residue was purified by silica gel column chromatography, eluting with PE/EtOAc (1:1) to afford 5-chloro-N-(2,4-difluoro-3-[[(1-[[2-(trimethylsilyl)ethoxy]methyl]pyrazolo[3,4-b]pyridin-5-yl)oxy]methyl]phenyl)-2-methoxypyridine-3-sulfonamide (360 mg, 92% yield) as a yellow solid.

LCMS (ES, m/z): [M+H]⁺: 612 Synthesis of Compound 1: 5-chloro-N-[2,4-difluoro-3-([1H-pyrazolo[3,4-b]pyridin-5-yloxy]methyl)phenyl]-2-methoxypyridine-3-sulfonamide

To a stirred solution of 5-chloro-N-(2,4-difluoro-3-[[(1-[[2-(trimethylsilyl)ethoxy]methyl]pyrazolo[3,4-b]pyridin-5-yl)oxy]methyl]phenyl)-2-methoxypyridine-3-sulfonamide (250 mg, 0.4 mmol, 1 equiv) in DCM (0.5 mL) was added TFA (0.5 mL, 6.7 mmol, 16 equiv) dropwise at room temperature under nitrogen atmosphere. The resulting mixture was stirred for 1 h, then quenched with sat. NaHCO₃ (aq.). The organics were dried over sodium sulphate, concentrated under vacuum and the residue was purified by prep-HPLC with the following conditions: Column, welch Vltimate XB—C18, 50×250 mm, 10 μm mobile phase, Mobile Phase A: 0.1% FA in Water, Mobile Phase B: CAN (10% up to 60% in 10 min) Detector, 220 nm. 5-Chloro-N-[2,4-difluoro-3-([1H-pyrazolo[3,4-b]pyridin-5-yloxy]methyl)phenyl]-2-methoxypyridine-3-sulfonamide (73 mg, 37% yield) was isolated as a white solid.

LCMS (ES, m/z): [M+H]⁺: 482

### ¹H NMR (300 MHz, DMSO-d6) δ 13.55 (s, 1H), 10.45 (s, 1H), 8.44 (d, J=2.6 Hz, 1H), 8.23 (d, J=2.7 Hz, 1H), 8.08-7.98 (m, 2H), 7.84 (d, J=2.8 Hz, 1H), 7.35 (td, J=9.0, 6.0 Hz, 1H), 7.11 (t, J=8.9 Hz, 1H), 5.12 (s, 2H), 3.90 (s, 3H).

### Example 13: Synthesis of N-[2,4-difluoro-3-[([3-methyl-1H-pyrazolo[3,4-b]pyridin-5-yl]oxy)methyl]phenyl]-5-fluoro-2-methoxypyridine-3-sulfonamide (Compound 2)

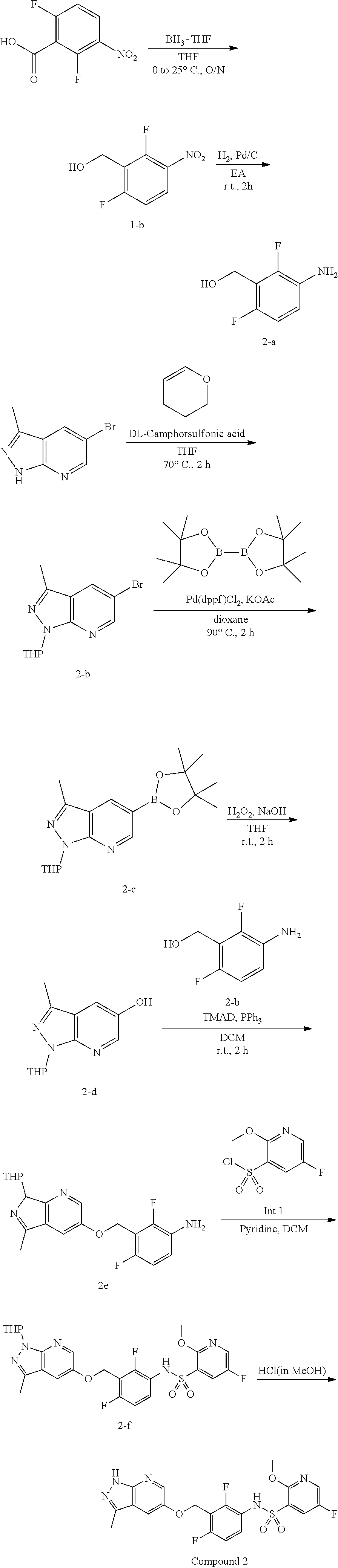

### Synthesis of 2-a: (3-amino-2,6-difluorophenyl)methanol

To a solution of (2,6-difluoro-3-nitrophenyl)methanol (11.6 g, 61 mmol, 1 equiv) in EtOAc (240 mL) was added Pd/C (10%, 3.8 g). The mixture was hydrogenated at room temperature for 2 h under hydrogen atmosphere using a hydrogen balloon. After completion, the reaction was filtered through a Celite pad and the filtrate was concentrated under reduced pressure to afford crude (3-amino-2,6-difluorophenyl)methanol (9.5 g, 97% yield) as a light brown solid which was used in next step directly without further purification.

LCMS (ES, m/z): [M+H]⁺: 160.

### Synthesis of 2-b: 5-bromo-3-methyl-1-(oxan-2-yl)pyrazolo[3,4-b]pyridine

A solution of 5-bromo-3-methyl-1H-pyrazolo[3,4-b]pyridine (5 g, 23.5 mmol, 1 equiv), dihydropyran (9.9 g, 117 mmol, 5 equiv) and DL-camphorsulfonic acid (0.8 g, 3.6 mmol, 0.15 equiv) in THE (50 mL) was stirred for 2 h at 70° C. The mixture was allowed to cool to room temperature. The resulting mixture was diluted with EtOAc (250 mL) and washed with 100 mL of aqueous NaHCO₃. The organic layer was washed with water (2×100 mL) and brine (100 mL), then dried over anhydrous Na₂SO₄. After filtration, the filtrate was concentrated under reduced pressure. The residue was purified by silica gel column chromatography, eluting with EtOAc/PE (1:4) to afford 5-bromo-3-methyl-1-(oxan-2-yl)pyrazolo[3,4-b]pyridine (6.7 g, 96% yield) as a colorless solid.

### Synthesis of 2-c: 3-methyl-1-(tetrahydro-2H-pyran-2-yl)-5-(4,4,5,5-tetramethyl-1,3,2-dioxaborolan-2-yl)-1H-pyrazolo[3,4-b]pyridine

A solution of 5-bromo-3-methyl-1-(oxan-2-yl)pyrazolo[3,4-b]pyridine (7 g, 23.6 mmol, 1 equiv), KOAc (4.6 g, 47.2 mmol, 2 equiv), Pd(dppf)Cl₂·CH₂Cl₂ (96 mg, 1.18 mmol, 0.05 equiv) and bis(pinacolato)diboron (9 g, 35.5 mmol, 1.5 equiv) in dioxane (70 mL) was stirred at 90° C. for 2 h under nitrogen atmosphere. The mixture was allowed to cool to room temperature; the resulting mixture was used in the next step directly without further purification.

LCMS (ES, m/z): [M+H]⁺: 344.

### Synthesis of 2-d: 3-methyl-1-(oxan-2-yl)pyrazolo[3,4-b]pyridin-5-ol

To the reaction solution of (3-methyl-1-(oxan-2-yl)-5-(4,4,5,5-tetramethyl-1,3,2-dioxaborolan-2-yl) pyrazolo[3,4-b]pyridine (23.6 mmol, 1 equiv) were added THE (80 mL), NaOH (2%, 80 mL) and H₂O₂ (30%, 8 g, 70.8 mmol, 3 equiv) dropwise at room temperature. The resulting mixture was stirred at room temperature for 2 h under air atmosphere. The reaction was quenched with aqueous Na₂S₂O₄ (200 mL). The resulting mixture was extracted with EtOAc (3×150 mL). The combined organic layers were washed with water (2×150 mL) and brine (150 mL), then dried over anhydrous Na₂SO₄. After filtration, the filtrate was concentrated under reduced pressure. The residue was purified by silica gel column chromatography, eluting with EtOAc/PE (1:1) to afford 3-methyl-1-(oxan-2-yl)pyrazolo[3,4-b]pyridin-5-ol (4.5 g, 81% yield) as a white solid.

LCMS (ES, m/z): [M+H]⁺: 234.

### Synthesis of 2-e: 2,4-difluoro-3-([[3-methyl-1-(oxan-2-yl)pyrazolo[3,4-b]pyridin-5-yl]oxy]methyl)aniline

A solution of 3-methyl-1-(oxan-2-yl)pyrazolo[3,4-b]pyridin-5-ol (5.4 g, 23 mmol, 1 equiv), TMAD (5.1 g, 29 mmol, 1.3 equiv), (3-amino-2,6-difluorophenyl)methanol (4 g, 25 mmol, 1.1 equiv) and PPh₃ (7.8 g, 29 mmol, 1.3 equiv) in DCM (60 mL) was stirred at room temperature for 2 h under nitrogen atmosphere. The resulting mixture was diluted with water (100 mL) and extracted with DCM (3×100 mL). The combined organic layers were washed with water (2×150 mL) and brine (150 mL), then dried over anhydrous Na₂SO₄. After filtration, the filtrate was concentrated under reduced pressure. The residue was purified by silica gel column chromatography, eluting with EtOAc/PE (1:1) to afford 2,4-difluoro-3-([[3-methyl-1-(oxan-2-yl)pyrazolo[3,4-b]pyridin-5-yl]oxy]methyl)aniline (6.4 g, 74% yield) as a white solid.

LCMS (ES, m/z): [M+H]⁺: 375.

### Synthesis of 2-f: N-[2,4-difluoro-3-([[3-methyl-1-(oxan-2-yl)pyrazolo[3,4-b]pyridin-5-yl]oxy]methyl)phenyl]-5-fluoro-2-methoxypyridine-3-sulfonamide

To a stirred solution of 2,4-difluoro-3-([[3-methyl-1-(oxan-2-yl)pyrazolo[3,4-b]pyridin-5-yl]oxy]methyl)aniline (5.5 g, 14.7 mmol, 1 equiv) and pyridine (4.65 g, 58 mmol, 4 equiv) in DCM (110 mL) was added 5-fluoro-2-methoxypyridine-3-sulfonyl chloride (4.97 g, 22 mmol, 1.5 equiv) in portions at room temperature. The resulting mixture was stirred for 4 h at room temperature, then diluted with water (100 mL). The mixture was extracted with DCM (3×100 mL) and the combined organic layers were washed with water (2×150 mL) and brine (150 mL), then dried over anhydrous Na₂SO₄. After filtration, the filtrate was concentrated under reduced pressure. The residue was purified by silica gel column chromatography, eluting with EtOAc/PE (0-50%) to afford N-[2,4-difluoro-3-([[3-methyl-1-(oxan-2-yl)pyrazolo[3,4-b]pyridin-5-yl]oxy]methyl)phenyl]-5-fluoro-2-methoxypyridine-3-sulfonamide (6.4 g, 77% yield) as a yellow solid.

LCMS (ES, m/z): [M+H]⁺: 564.

### Synthesis of Compound 2: N-[2,4-difluoro-3-[([3-methyl-1H-pyrazolo[3,4-b]pyridin-5-yl]oxy)methyl]phenyl]-5-fluoro-2-methoxypyridine-3-sulfonamide

To a stirred mixture of N-[2,4-difluoro-3-([[3-methyl-1-(oxan-2-yl)pyrazolo[3,4-b]pyridin-5-yl]oxy]methyl)phenyl]-5-fluoro-2-methoxypyridine-3-sulfonamide (6.4 g, 11.6 mmol, 1 equiv) was added HCl in MeOH (64 mL, 256 mmol, 22 equiv, 4 M) dropwise at 0-5° C. The resulting mixture was stirred for 1 h at room temperature. After concentration under vacuum, the residue was neutralized to pH 7 with NH₃·H₂O (5%) and this was dissolved in DMF (30 mL). The crude product was purified by Prep-HPLC with the following conditions: Column: Welch XB—C18, 50×250 mm, mobile phase: 200 mL/min, Mobile Phase A: 0.1% FA in Water, Mobile Phase B: ACN (20% up to 65% in 30 min). N-[2,4-Difluoro-3-[([3-methyl-1H-pyrazolo[3,4-b]pyridin-5-yl]oxy)methyl]phenyl]-5-fluoro-2-methoxypyridine-3-sulfonamide (5.1 g, 94%) was isolated as a white solid.

LCMS (ES, m/z): [M+H]⁺: 480.
¹H NMR (300 MHz, DMSO-d6) δ 13.09 (s, 1H), 10.36 (s, 1H), 8.44 (d, J=3.0 Hz, 1H), 8.18 (d, J=2.7 Hz, 1H), 7.98 (dd, J=7.3, 3.0 Hz, 1H), 7.86 (d, J=2.8 Hz, 1H), 7.38 (td, J=8.9, 6.0 Hz, 1H), 7.16 (td, J=9.0, 1.6 Hz, 1H), 5.14 (s, 2H), 3.91 (s, 3H), 2.48 (s, 3H).

### Example 14: Synthesis of 5-chloro-N-[2,4-difluoro-3-([1H-pyrazolo[3,4-b]pyridin-5-ylamino]methyl)phenyl]-2-methoxypyridine-3-sulfonamide (Compound 3)

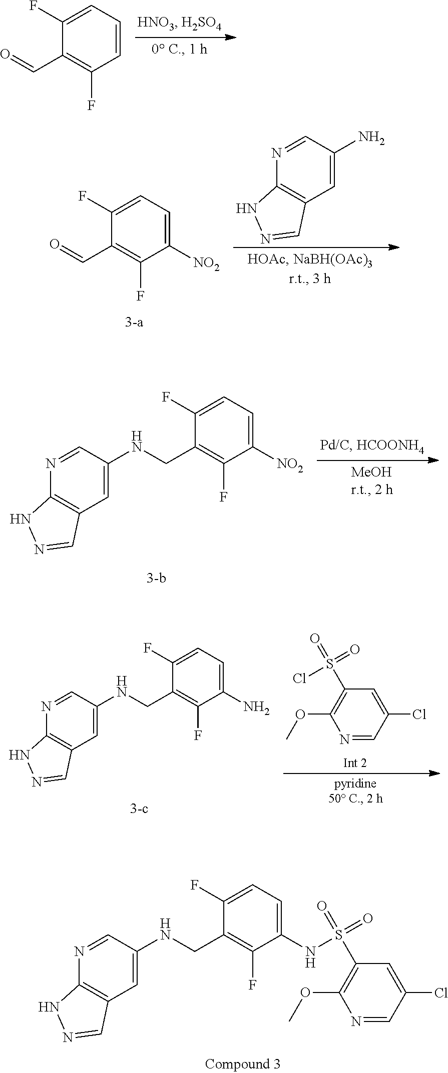

### Synthesis of 3-a: 2,6-difluoro-3-nitrobenzaldehyde

To a stirred solution of 2,6-difluorobenzaldehyde (1.0 g, 7.0 mmol, 1 equiv) in H₂SO₄ (2.5 mL) was added HNO₃ (1.0 mL, 68%) dropwise at 0° C. under air atmosphere. The resulting mixture was stirred for 1 h at 0° C. and then poured onto ice water. The resulting mixture was filtered and the filter cake washed with water (2×10 mL). The resulting solid was dried under infrared light to afford crude 2,6-difluoro-3-nitrobenzaldehyde (1 g, 76% yield) as a yellow solid which was used in next step directly without further purification.

### Synthesis of 3-b: N-[(2,6-difluoro-3-nitrophenyl)methyl]-1H-pyrazolo[3,4-b]pyridin-5-amine

NaBH(OAc)₃ (566 mg, 2.6 mmol, 2 equiv) was added portion wise to a stirred solution of 2,6-difluoro-3-nitrobenzaldehyde (250 mg, 1.3 mmol, 1 equiv) and 1H-pyrazolo[3,4-b]pyridin-5-amine (179 mg, 1.3 mmol, 1 equiv) in MeOH (10 mL) and HOAc (8 mg, 0.13 mmol, 0.1 equiv) at room temperature under nitrogen atmosphere. The reaction mixture was stirred for 3 h and then concentrated under reduced pressure. The residue was purified by silica gel column chromatography, eluting with PE/EtOAc (5:1) to afford N-[(2,6-difluoro-3-nitrophenyl)methyl]-1H-pyrazolo[3,4-b]pyridin-5-amine (250 mg, 61% yield) as a yellow solid.

LCMS (ES, m/z): [M+H]⁺: 306

### Synthesis of 3-c: N-[(3-amino-2,6-difluorophenyl)methyl]-1H-pyrazolo[3,4-b]pyridin-5-amine

Pd/C (14 mg, 0.013 mmol, 0.02 equiv, 10% w/w) was added portion wise to a stirred solution of N-[(2,6-difluoro-3-nitrophenyl)methyl]-1H-pyrazolo[3,4-b]pyridin-5-amine (200 mg, 0.65 mmol, 1 equiv) and HCOONH₄ (206 mg, 3.3 mmol, 5 equiv) in MeOH (10 mL) at room temperature. The reaction mixture was stirred for 2 h at room temperature and then filtered. The filter cake was washed with MeOH (2×10 mL) and the filtrate concentrated under reduced pressure. The residue was purified by silica gel column chromatography, eluting with PE/EtOAc (1:1) to afford N-[(3-amino-2,6-difluorophenyl)methyl]-1H-pyrazolo[3,4-b]pyridin-5-amine (160 mg, 88% yield) as a yellow solid.

LCMS (ES, m/z): [M+H]⁺: 276

### Synthesis of Compound 3: 5-chloro-N-[2,4-difluoro-3-([1H-pyrazolo[3,4-b]pyridin-5-ylamino]methyl)phenyl]-2-methoxypyridine-3-sulfonamide

To a stirred solution of N-[(3-amino-2,6-difluorophenyl)methyl]-1H-pyrazolo[3,4-b]pyridin-5-amine (200 mg, 0.73 mmol, 1 equiv) in pyridine (6 mL) was added 5-chloro-2-methoxypyridine-3-sulfonyl chloride (350 mg, 1.5 mmol, 2 equiv) portion wise at room temperature under a nitrogen atmosphere. The reaction was stirred for 2 h at 50° C. and then concentrated under reduced pressure. The crude product was purified by prep-HPLC with the following conditions: Column: welch Vltimate XB—C18, 50×250 mm, 10 μm; Mobile Phase: 10-50% MeCN/0.1% aqueous formic acid; Detector: 220 nm; to afford 5-chloro-N-[2,4-difluoro-3-([1H-pyrazolo[3,4-b]pyridin-5-ylamino]methyl)phenyl]-2-methoxypyridine-3-sulfonamide (57 mg, 16% yield) as a white solid.

LCMS (ES, m/z): [M+H]⁺: 481
¹H NMR (300 MHz, DMSO-d6) δ 13.17 (s, 1H), 10.31 (s, 1H), 8.33 (d, J=2.6 Hz, 1H), 8.10 (d, J=2.6 Hz, 1H), 7.97 (d, J=2.6 Hz, 1H), 7.84 (d, J=1.4 Hz, 1H), 7.17 (d, J=7.1 Hz, 2H), 6.96 (t, J=9.1 Hz, 1H), 5.99 (t, J=5.9 Hz, 1H), 4.21 (d, J=5.7 Hz, 2H), 3.85 (s, 3H).

### Example 15: Synthesis of 5-chloro-N-(2,4-difluoro-3-[1H-pyrazolo[3,4-b]pyridin-5-ylmethoxy]phenyl)-2-methoxypyridine-3-sulfonamide (Compound 4)

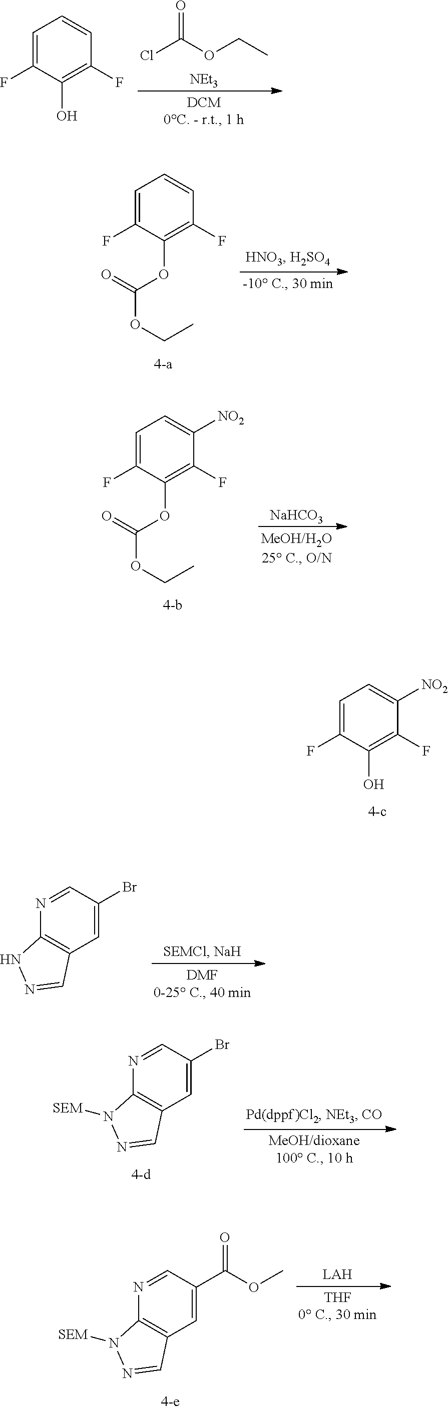

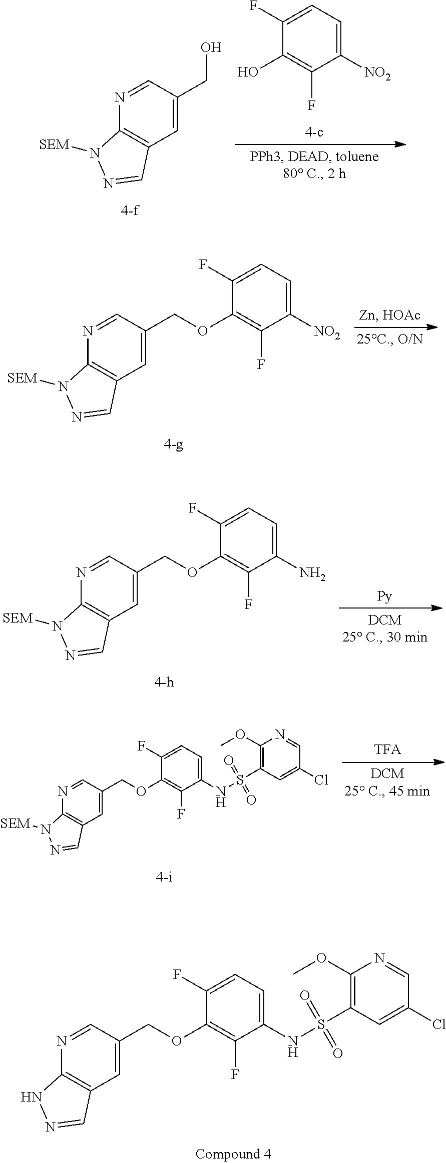 [SMILES: C[Si](C)(C)CCOCn1ncc2cc(CO)cnc21 ; O=[N+]([O-])c1ccc(F)c(O)c1F ; C[Si](C)(C)CCOCn1ncc2cc(COc3c(F)ccc([N+](=O)[O-])c3F)cnc21 ; C[Si](C)(C)CCOCn1ncc2cc(COc3c(F)ccc(N)c3F)cnc21 ; COc1ncc(Cl)cc1S(=O)(=O)Nc1ccc(F)c(OCc2cnc3c(cnn3COCC[Si](C)(C)C)c2)c1F ; COc1ncc(Cl)cc1S(=O)(=O)Nc1ccc(F)c(OCc2cnc3[nH]ncc3c2)c1F]

### Synthesis of 4-a: 2,6-difluorophenyl ethyl carbonate

Ethyl carbonochloridate (3.7 g, 34 mmol, 1.5 equiv) was added to a solution of 2,6-difluorophenol (3.0 g, 23 mmol, 1 equiv) in DCM (30 mL) and NEt₃ (4.7 g, 46 mmol, 2 equiv) at 0° C. The resulting solution was stirred at room temperature for 1 h and then quenched by the addition of ice water (10 mL). The resulting mixture was extracted with DCM (3×40 mL), dried over anhydrous Na₂SO₄, filtered and concentrated under reduced pressure to afford crude 2,6-difluorophenyl ethyl carbonate (3.6 g, 77% yield) as a light yellow oil, which was used directly in next step without further purification.

LCMS (ES, m/z): [M+H]⁺: 203

### Synthesis of 4-b: 2,6-difluoro-3-nitrophenyl ethyl carbonate

HNO₃ (13 mL) was added to a solution of 2,6-difluorophenyl ethyl carbonate (3.5 g, 17 mmol, 1 equiv) in H₂SO₄ (5 mL) at 0° C. The resulting solution was stirred at −10° C. for 30 min and then diluted with H₂O (20 mL). The resulting solution was extracted with EtOAc (3×20 mL), dried over anhydrous Na₂SO₄, filtered and concentrated under reduced pressure to afford crude 2,6-difluoro-3-nitrophenyl ethyl carbonate (4.8 g) as light yellow oil, which was used directly in next step without further purification.

LCMS (ES, m/z): [M+H]⁺: 248

### Synthesis of 4-c: 2,6-difluoro-3-nitrophenol

Into a 250 mL round-bottom flask, was placed 2,6-difluoro-3-nitrophenyl ethyl carbonate (4.7 g, 19 mmol, 1 equiv), NaHCO₃ (2.4 g, 28 mmol, 1.5 equiv), MeOH (20 mL) and H₂O (20 mL). The resulting solution was stirred at room temperature overnight. The solids were filtered and the filtrate was concentrated under reduced pressure. The residue was purified by silica gel chromatography, eluting with PE/EtOAc (3:1), to afford 2,6-difluoro-3-nitrophenol (2.3 g, 69% yield) as a yellow solid.

LCMS (ES, m/z): [M+H]⁺: 176

### Synthesis of 4-d: 5-bromo-1-[[2-(trimethylsilyl)ethoxy]methyl]pyrazolo[3,4-b]pyridine

NaH (0.6 g, 25.2 mmol, 2 equiv) was added portion wise to a solution of 5-bromo-1H-pyrazolo[3,4-b]pyridine (2.5 g, 12.6 mmol, 1 equiv) in DMF (50 mL) at 0° C. SEMCl (3.2 g, 19 mmol, 1.5 equiv) was added at 0° C. and the reaction mixture stirred at room temperature for 40 minutes, then quenched with ice water (100 mL). The mixture was extracted with EA (3×100 mL), dried over anhydrous Na₂SO₄, filtered, and concentrated under reduced pressure. The residue was purified by silica gel chromatography, eluting with PE/EA (5:1), to afford 5-bromo-1-[[2-(trimethylsilyl)ethoxy]methyl]pyrazolo[3,4-b]pyridine (2.1 g, 50% yield) as light yellow oil.

LCMS (ES, m/z): [M+H]⁺: 328

### Synthesis of 4-e: methyl-1-[[2-(trimethylsilyl)ethoxy]methyl]pyrazolo[3,4-b]pyridine-5-carboxylate

Into a 50 mL sealed tube purged and maintained with an inert atmosphere of nitrogen was placed 5-bromo-1-[[2-(trimethylsilyl)ethoxy]methyl]pyrazolo[3,4-b]pyridine (2.1 g, 6.4 mmol, 1 equiv), Pd(dppf)Cl₂ (0.94 g, 1.28 mmol, 0.2 equiv), TEA (3.2 g, 32 mmol, 5 equiv), MeOH (20 mL), dioxane (5 mL), and CO. The resulting solution was stirred at 100° C. for 10 h. The reaction mixture was cooled to room temperature and concentrated under reduced pressure. The residue was purified by silica gel chromatography, eluting with PE/EA (7:1), to afford methyl-1-[[2-(trimethylsilyl)ethoxy]methyl]pyrazolo[3,4-b]pyridine-5-carboxylate (1.4 g, 71% yield) as light yellow oil.

LCMS (ES, m/z): [M+H]⁺: 308

### Synthesis of 4-f: (1-[[2-(trimethylsilyl)ethoxy]methyl]pyrazolo[3,4-b]pyridin-5-yl)methanol

LAH (0.18 g, 4.7 mmol, 1.2 equiv) was added to a stirred solution of methyl-1-[[2-(trimethylsilyl)ethoxy]methyl]pyrazolo[3,4-b]pyridine-5-carboxylate (1.2 g, 3.9 mmol, 1 equiv) in THE (10 mL) at 0° C. The reaction mixture was stirred at 0° C. for 30 min and then quenched by the addition of ice water (15 mL). The solution was adjusted to pH 9 with aqueous NaOH (2 M, 10 mL). The mixture was extracted with EA (3×50 mL), dried over anhydrous Na₂SO₄, filtered and concentrated under reduced pressure. The residue was purified by silica gel chromatography, eluting with PE/EA (5:1), to afford (1-[[2-(trimethylsilyl)ethoxy]methyl]pyrazolo[3,4-b]pyridin-5-yl)methanol (460 mg, 42% yield) as light yellow oil.

LCMS (ES, m/z): [M+H]⁺: 280

### Synthesis of 4-g: 5-(2,-difluoro-3-nitrophenoxymethyl)-1-[[2-(trimethylsilyl)ethoxy]methyl]pyrazolo[3,4-b]pyridine

PPh₃ (470 mg, 1.8 mmol, 1.2 equiv) and DEAD (390 mg, 2.2 mmol, 1.5 equiv) were added to a solution of (1-[[2-(trimethylsilyl)ethoxy]methyl]pyrazolo[3,4-b]pyridin-5-yl)methanol (42-mg, 1.5 mmol, 1 equiv) and 2,6-difluoro-3-nitrophenol (260 mg, 1.5 mmol, 1 equiv) in toluene (12 mL) under an atmosphere of nitrogen. The reaction mixture was stirred at 80° C. for 2 h and then concentrated. The residue was purified by silica gel chromatography, eluting with PE/EA (2:1), to afford 5-(2,-difluoro-3-nitrophenoxymethyl)-1-[[2-(trimethylsilyl)ethoxy]methyl]pyrazolo[3,4-b]pyridine (320 mg, 48% yield) as a brown oil.

LCMS (ES, m/z): [M+H]⁺: 437

### Synthesis of 4-h: 2,4-difluoro-3-[(1-[[2-(trimethylsilyl)ethoxy]methyl]pyrazolo[3,4-b]pyridin-5-yl)methoxy]aniline

Zn (90 mg, 1.4 mmol, 2 equiv) was added to a solution of 5-(2,6-difluoro-3-nitrophenoxymethyl)-1-[[2-(trimethylsilyl)ethoxy]methyl]pyrazolo[3,4-b]pyridine (300 mg, 0.7 mmol, 1 equiv) in HOAc (6 mL) and then stirred at 25° C. overnight. The resulting mixture was concentrated and the residue diluted with water (5 mL). The solution was adjusted to pH 8 with saturated aqueous NaHCO₃ and then extracted with EA (3×15 mL). The combined organic extracts were dried over anhydrous Na₂SO₄, filtered, and concentrated under reduced pressure. The residue was purified by silica gel chromatography, eluting with PE/EA (1:1), to afford 2,4-difluoro-3-[(1-[[2-(trimethylsilyl)ethoxy]methyl]pyrazolo[3,4-b]pyridin-5-yl)methoxy]aniline (138 mg, 49% yield) as a yellow solid.

LCMS (ES, m/z): [M+H]⁺: 407

### Synthesis of 4-i: 5-chloro-N-[2,4-difluoro-3-[(1-[[2-(trimethylsilyl)ethoxy]methyl]pyrazolo[3,4-b]pyridin-5-yl)methoxy]phenyl]-2-methoxypyridine-3-sulfonamide

Into an 8 mL vial was placed 2,4-difluoro-3-[(1-[[2-(trimethylsilyl)ethoxy]methyl]pyrazolo[3,4-b]pyridin-5-yl)methoxy]aniline (120 mg, 0.3 mmol, 1 equiv), DCM (3 mL), pyridine (230 mg, 3 mmol, 10 equiv) and 5-chloro-2-methoxypyridine-3-sulfonyl chloride (110 mg, 0.45 mmol, 1.5 equiv). The reaction mixture was stirred at 25° C. for 30 min and then diluted with water (15 mL). The mixture was extracted with DCM (3×15 mL) and the combined organic extracts dried over anhydrous Na₂SO₄, filtered, and concentrated under reduced pressure to afford crude 5-chloro-N-[2,4-difluoro-3-[(1-[[2-(trimethylsilyl)ethoxy]methyl]pyrazolo[3,4-b]pyridin-5-yl)methoxy]phenyl]-2-methoxypyridine-3-sulfonamide (150 mg, 83% yield) as a yellow solid.

LCMS (ES, m/z): [M+H]⁺: 612

### Synthesis of Compound 4: 5-chloro-N-(2,4-difluoro-3-[1H-pyrazolo[3,4-b]pyridin-5-ylmethoxy]phenyl)-2-methoxypyridine-3-sulfonamide

A solution of 5-chloro-N-[2,4-difluoro-3-[(1-[[2-(trimethylsilyl)ethoxy]methyl]pyrazolo[3,4-b]pyridin-5-yl)methoxy]phenyl]-2-methoxypyridine-3-sulfonamide (130 mg) in DCM (2 mL) and TFA (2 mL) was stirred at room temperature for 45 min and then then quenched with ice water (10 mL). The solution was adjusted to pH 8 with saturated aqueous NaHCO₃ and extracted with DCM (3×10 mL). The combined organic extracts were dried over anhydrous Na₂SO₄, filtered, and concentrated. The residue was purified by prep-HPLC with the following conditions: Column: welch Vltimate XB—C18, 50×250 mm, 10 μm; Mobile Phase: 30-50% MeCN/0.1% aqueous formic acid; Detector: 220 nm; to afford 5-chloro-N-(2,4-difluoro-3-[1H-pyrazolo[3,4-b]pyridin-5-ylmethoxy]phenyl)-2-methoxypyridine-3-sulfonamide (32 mg, 31% yield) as a white solid.

LCMS (ES, m/z): [M+H]⁺: 482

### ¹H NMR (300 MHz, DMSO-d6) δ 13.72 (s, 1H), 10.31 (s, 1H), 8.46 (s, 2H), 8.12 (s, 2H), 7.92 (s, 1H), 7.05 (d, J=10.2 Hz, 1H), 6.91 (s, 1H), 5.19 (s, 2H), 3.91 (s, 3H).

### Example 16: Synthesis of 5-chloro-N-[2,4-difluoro-3-(2-[5H,6H,8H-imidazo[2,1-c][1,4]oxazin-3-yl]ethyl)phenyl]-2-methoxypyridine-3-sulfonamide (Compound 5)

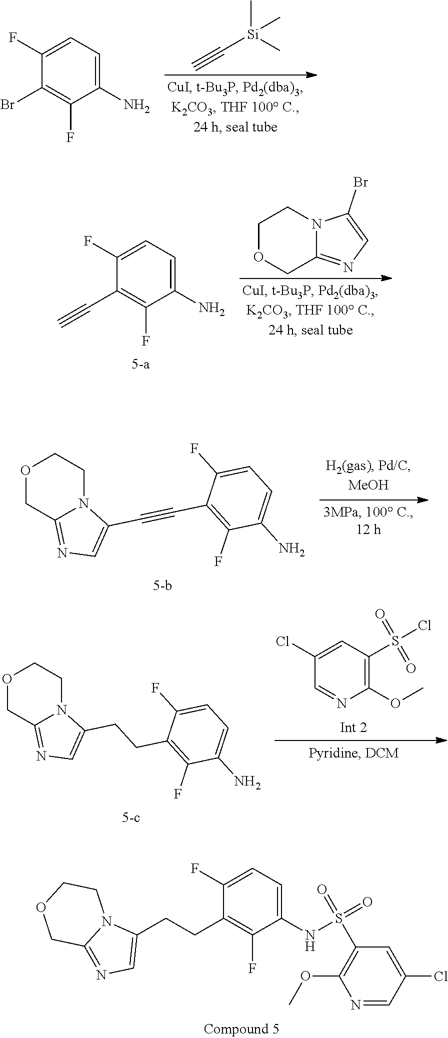

### Synthesis of 5-a: 3-ethynyl-2,4-difluoroaniline

Into a 50 mL sealed tube purged and maintained with an inert atmosphere of nitrogen, were placed 3-bromo-2,4-difluoroaniline (2 g, 9.7 mmol, 1 equiv), trimethylsilylacetylene (1.4 g, 14.5 mmol, 1.5 equiv), CuI (180 mg, 0.97 mmol, 0.1 equiv), Pd₂(dba)₃ (560 mg, 0.48 mmol, 0.05 equiv), K₂CO₃ (4.0 g, 29 mmol, 3 equiv), t-Bu₃P/Toluene (390 mg, 0.97 mmol, 0.1 equiv), and THE (15 mL). The resulting solution was stirred for 24 h at 100° C. and then concentrated under reduced pressure. The residue was purified by silica gel column chromatography, eluting with PE:EA (10:1) to afford 3-ethynyl-2,4-difluoroaniline (900 mg, 70% yield) as a red solid.

### Synthesis of 5-b: 2,4-difluoro-3-(2-[5H,6H,8H-imidazo[2,1-c][1,4]oxazin-3-yl]ethynyl)aniline

Into a 30 mL sealed tube purged and maintained with an inert atmosphere of nitrogen, were placed 3-ethynyl-2,4-difluoroaniline (300 mg, 2.0 mmol, 1 equiv), 3-bromo-5H,6H,8H-imidazo[2,1-c][1,4]oxazine (400 mg, 2.0 mmol, 1 equiv), CuI (37 mg, 0.2 mmol, 0.1 equiv), Pd₂(dba)₃ (110 mg, 0.1 mmol, 0.05 equiv), K₂CO₃ (810 mg, 5.9 mmol, 3 equiv), t-Bu₃P/Toluene (79 mg, 0.2 mmol, 0.1 equiv) and THE (5 mL). The reaction mixture was stirred for 24 h at 100° C. and then concentrated under reduced pressure. The residue was purified by silica gel column chromatography, eluting with PE:EA (1:1) to afford 2,4-difluoro-3-(2-[5H,6H,8H-imidazo[2,1-c][1,4]oxazin-3-yl]ethynyl)aniline (190 mg, 80% yield) as a light yellow solid.

LCMS (ES, m/z): [M+H]⁺:276

### Synthesis of 5-c: 2,4-difluoro-3-(2-[5H,6H,8H-imidazo[2,1-c][1,4]oxazin-3-yl]ethyl)aniline

Into a 25 mL pressure tank reactor, was placed 2,4-difluoro-3-(2-[5H,6H,8H-imidazo[2,1-c][1,4]oxazin-3-yl]ethynyl)aniline (70 mg, 0.25 mmol, 1 equiv), 10% Pd/C (7 mg, 0.025 mmol, 0.1 equiv) and MeOH (5 mL). The mixture was stirred at 100° C. for 12 hours under H₂ (30 atm). The mixture was filtered and the filtrate concentrated under reduced pressure to afford 2,4-difluoro-3-(2-[5H,6H,8H-imidazo[2,1-c][1,4]oxazin-3-yl]ethyl)aniline (70 mg, 90% yield) as a light yellow solid that was used directly in the next step without further purification.

LCMS (ES, m/z): [M+H]⁺: 280

### Synthesis of Compound 5: 5-chloro-N-[2,4-difluoro-3-(2-[5H,6H,8H-imidazo[2,1-c][1,4]oxazin-3-yl]ethyl)phenyl]-2-methoxypyridine-3-sulfonamide

5-Chloro-2-methoxypyridine-3-sulfonyl chloride (60 mg, 0.25 mmol, 1 equiv) was added to a solution of 2,4-difluoro-3-(2-[5H,6H,8H-imidazo[2,1-c][1,4]oxazin-3-yl]ethyl)aniline (70 mg, 0.25 mmol, 1 equiv) in pyridine (40 mg, 0.5 mmol, 2 equiv) and DCM (5 mL). The reaction mixture was stirred for 1 h at room temperature and then concentrated under reduced pressure. The crude product was purified by Prep-HPLC with the following conditions: Column: welch Vltimate XB—C18, 50×250 mm, 10 μm; Mobile Phase: 35-70% MeCN/0.05% aqueous NH₄HCO₃; Detector: 220 nm; to afford 5-chloro-N-[2,4-difluoro-3-(2-[5H,6H,8H-imidazo[2,1-c][1,4]oxazin-3-yl]ethyl)phenyl]-2-methoxypyridine-3-sulfonamide (78 mg, 97% yield) as a white solid.

LCMS (ES, m/z): [M+H]⁺: 485

### ¹H NMR (300 MHz, DMSO-d6) δ 10.28 (s, 1H), 8.51 (s, 1H), 8.00 (s, 1H), 7.12-6.98 (m, 2H), 6.48 (s, 1H), 4.66 (s, 2H), 3.96 (s, 5H), 3.82 (s, 2H), 2.81-2.50 (m, 4H).

### Example 17: Synthesis of 5-chloro-N-[2,4-difluoro-3-([1H-pyrazolo[3,4-b]pyridin-5-ylmethyl]amino)phenyl]-2-methoxypyridine-3-sulfonamide (Compound 6)

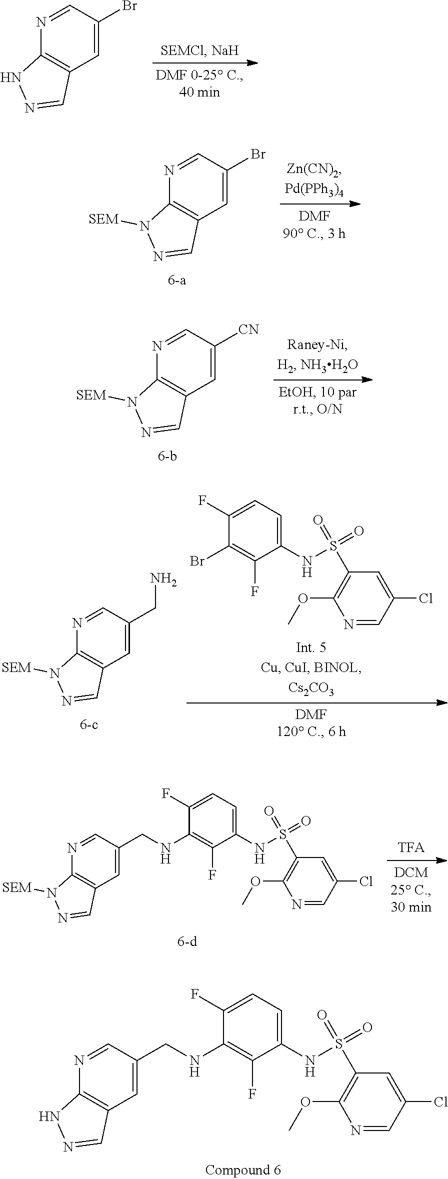

### Synthesis of 6-a: 5-bromo-1-[[2-(trimethylsilyl)ethoxy]methyl]pyrazolo[3,4-b]pyridine

NaH (1.2 g, 50 mmol, 2 equiv) was added portion wise to a solution of 5-bromo-1H-pyrazolo[3,4-b]pyridine (5.0 g, 25 mmol, 1 equiv) in DMF (100 mL) at 0° C. SEMCl (6.3 g, 38 mmol, 1.5 equiv) was then added at 0° C. and the reaction mixture stirred for 40 min at room temperature. The reaction was quenched with ice water (20 mL) and extracted with EA (3×200 mL). The combined organic extracts were dried over Na₂SO₄, filtered, and concentrated under reduced pressure. The residue was purified by silica gel column chromatography, eluting with PE:EA (5:1), to afford 5-bromo-1-[[2-(trimethylsilyl)ethoxy]methyl]pyrazolo[3,4-b]pyridine (5.2 g, 63% yield) as light yellow oil.

LCMS (ES, m/z): [M+H]⁺: 328

### Synthesis of 6-b: 1-[[2-(trimethylsilyl)ethoxy]methyl]pyrazolo[3,4-b]pyridine-5-carbonitrile

DMF (20 mL) was added to a flask containing 5-bromo-1-[[2-(trimethylsilyl)ethoxy]methyl]pyrazolo[3,4-b]pyridine (2.0 g, 6.0 mmol, 1 equiv), Zn(CN)₂ (1.0 g, 9.0 mmol, 1.5 equiv), Pd(PPh₃)₄ (1.4 g, 1.2 mmol, 0.2 equiv) under an atmosphere of N₂. The reaction mixture was stirred at 90° C. for 3 h and then concentrated under reduced pressure. The residue was purified by silica gel chromatography, eluting with PE:EA (7:1), to afford 1-[[2-(trimethylsilyl)ethoxy]methyl]pyrazolo[3,4-b]pyridine-5-carbonitrile (1.2 g, 74% yield) as brown oil.

LCMS (ES, m/z): [M+H]⁺: 275

### Synthesis of 6-c: 1-(1-[[2-(trimethylsilyl)ethoxy]methyl]pyrazolo[3,4-b]pyridin-5-yl)methanamine

Into a 50 mL sealed tube were placed 1-[[2-(trimethylsilyl)ethoxy]methyl]pyrazolo[3,4-b]pyridine-5-carbonitrile (1.1 g, 4.0 mmol, 1 equiv), EtOH (30 mL), NH₃·H₂O (0.5 mL), Raney-Ni (0.2 g) and the mixture was stirred at room temperature overnight under an atmosphere of H₂ (10 atm). The mixture was filtered carefully and the filtrate was concentrated to afford 1-(1-[[2-(trimethylsilyl)ethoxy]methyl]pyrazolo[3,4-b]pyridin-5-yl)methanamine (0.8 g, 72% yield) as a brown solid which was used in the next step without further purification.

LCMS (ES, m/z): [M+H]⁺: 279

### Synthesis of 6-d: 5-chloro-N-(2,4-difluoro-3-[[(1-[[2-(trimethylsilyl)ethoxy]methyl]pyrazolo[3,4-b]pyridin-5-yl)methyl]amino]phenyl)-2-methoxypyridine-3-sulfonamide

DMF (15 mL) was added to a 1-(1-[[2-(trimethylsilyl)ethoxy]methyl]pyrazolo[3,4-b]pyridin-5-yl)methanamine (500 mg, 1.8 mmol, 1 equiv), N-(3-bromo-2,4-difluorophenyl)-5-chloro-2-methoxypyridine-3-sulfonamide (740 mg, 1.8 mmol, 1 equiv), BINOL (770 mg, 2.7 mmol, 1.5 equiv), Cu (110 mg, 1.8 mmol, 1 equiv), CuI (340 mg, 1.8 mmol, 1 equiv), Cs₂CO₃ (1.2 g, 3.6 mmol, 2 equiv) under an atmosphere of N₂ and the resulting solution was stirred for at 120° C. for 6 h. The reaction mixture was cooled to room temperature and then filtered. The filtrate was concentrated under reduced pressure and the residue purified by prep-HPLC with the following conditions Column: welch Vltimate XB—C18, 50×250 mm, 10 μm; Mobile Phase: 10-65% MeCN/0.1% aqueous formic acid; Detector: 220 nm; to afford 5-chloro-N-(2,4-difluoro-3-[[(1-[[2-(trimethylsilyl)ethoxy]methyl]pyrazolo[3,4-b]pyridin-5-yl)methyl]amino]phenyl)-2-methoxypyridine-3-sulfonamide (72 mg, 6.6% yield) as a white solid.

LCMS (ES, m/z): [M+H]⁺: 611

### Synthesis of Compound 6: 5-chloro-N-[2,4-difluoro-3-([1H-pyrazolo[3,4-b]pyridin-5-ylmethyl]amino)phenyl]-2-methoxypyridine-3-sulfonamide

A solution of 5-chloro-N-(2,4-difluoro-3-[[(1-[[2-(trimethylsilyl)ethoxy]methyl]pyrazolo[3,4-b]pyridin-5-yl)methyl]amino]phenyl)-2-methoxypyridine-3-sulfonamide (59 mg, 0.097 mmol, 1 equiv) in DCM (2 mL) and TFA (2 mL) was stirred at room temperature for 30 min. The reaction mixture was quenched with ice water (10 mL). The solution was adjusted to pH 8 with saturated aqueous NaHCO₃ and extracted with DCM (3×10 mL). The combined organic extracts were dried over Na₂SO₄, filtered, and concentrated under reduced pressure. The residue was purified by prep-HPLC with the following conditions: Column: welch Vltimate XB—C18; 50×250 mm, 10 μm, Mobile Phase: 10-50% MeCN/0.1% aqueous formic acid; Detector: 220 nm; to afford 5-chloro-N-[2,4-difluoro-3-([1H-pyrazolo[3,4-b]pyridin-5-ylmethyl]amino)phenyl]-2-methoxypyridine-3-sulfonamide (7 mg, 15% yield) as a white solid.

LCMS (ES, m/z): [M+H]⁺: 481

### ¹H NMR (300 MHz, DMSO-d6) δ 13.56 (s, 1H), 10.03 (s, 1H), 8.45-8.37 (m, 2H), 8.08 (s, 1H), 7.99 (s, 1H), 7.91 (d, J=2.6 Hz, 1H), 6.81 (t, J=10.2 Hz, 1H), 6.56-6.40 (m, 1H), 6.03 (s, 1H), 4.43 (d, J=6.9 Hz, 2H), 3.88 (3H, s)

### Example 18: Synthesis of 5-chloro-N-[2,4-difluoro-3-(2-[1H-pyrazolo[3,4-b]pyridin-5 yl]ethyl)phenyl]-2-methoxypyridine-3-sulfonamide (Compound 7)

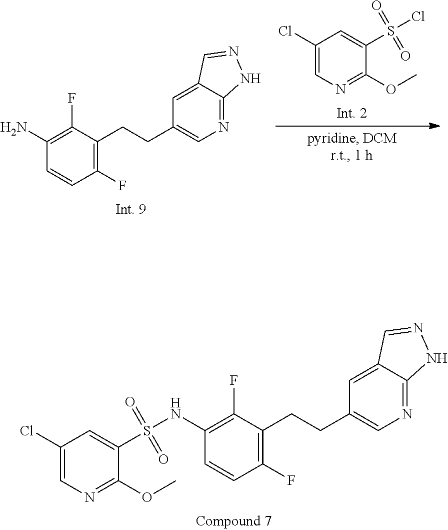 [SMILES: Nc1ccc(F)c(CCc2cnc3[nH]ncc3c2)c1F ; COc1ncc(Cl)cc1S(=O)(=O)Cl ; COc1ncc(Cl)cc1S(=O)(=O)Nc1ccc(F)c(CCc2cnc3[nH]ncc3c2)c1F]

### Synthesis of Compound 7: 5-chloro-N-[2,4-difluoro-3-(2-[1H-pyrazolo[3,4-b]pyridin-5-yl]ethyl)phenyl]-2-methoxypyridine-3-sulfonamide

To a solution of 2,4-difluoro-3-(2-[1H-pyrazolo[3,4-b]pyridin-5-yl]ethyl)aniline (500 mg, 1.8 mmol, 1 equiv) in DCM (10 mL) was added pyridine (430 mg, 5.5 mmol, 3 equiv) and 5-chloro-2-methoxypyridine-3-sulfonyl chloride (660 mg, 2.7 mmol, 1.5 eq). The reaction was stirred at room temperature for 1 h and then the solution was concentrated. The residue was purified by prep-HPLC with the following conditions: Column: welch Vltimate XB—C18, 50×250 mm, 10 μm; Mobile Phase 10-65% MeCN/0.1% aqueous formic acid; Detector: 220 nm; to afford 5-chloro-N-[2,4-difluoro-3-(2-[1H-pyrazolo[3,4-b]pyridin-5-yl]ethyl)phenyl]-2-methoxypyridine-3-sulfonamide (200 mg, 22% yield) as a white solid.

LCMS (ES, m/z): [M+H]⁺: 480
¹H NMR (300 MHz, DMSO-d₆) δ 13.51 (s, 1H), 10.23 (s, 1H), 8.51 (d, J=2.6 Hz, 1H), 8.21 (d, J=2.1 Hz, 1H), 8.03 (d, J=1.2 Hz, 1H), 7.99 (d, J=2.6 Hz, 1H), 7.83 (d, J=2.1 Hz, 1H), 7.11 (td, J=8.9, 5.9 Hz, 1H), 6.94 (td, J=9.0, 1.6 Hz, 1H), 3.95 (s, 3H), 2.87 (m, 4H).

### Example 19: Synthesis of 5-[2-[3-(5-chloro-2-methoxypyridine-3-sulfonamido)-2,6-difluorophenyl]ethyl]-N-methyl-1H-pyrazole-3-carboxamide (Compound 8)

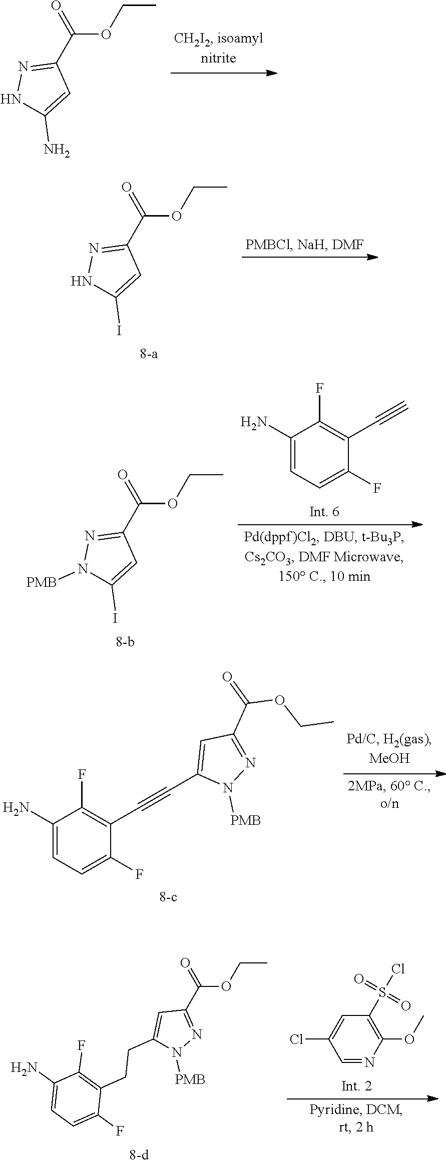

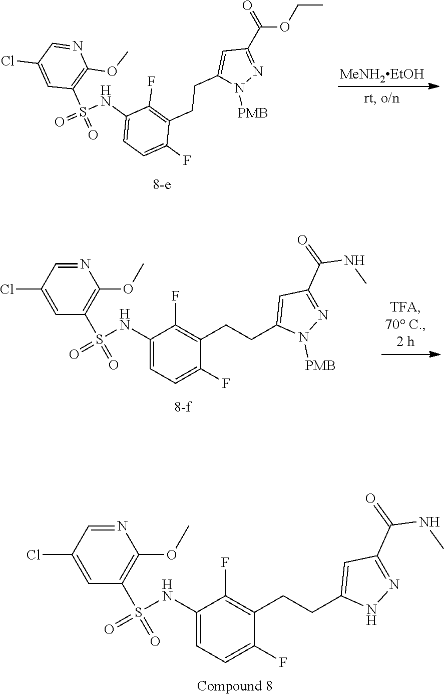

### Synthesis of 8-a: ethyl 5-iodo-1H-pyrazole-3-carboxylate

To a suspension of ethyl 3-amino-1H-pyrazole-5-carboxylate (10 g) in diiodomethane (200 mL) at −10° C. under N₂ atmosphere, was slowly added isoamyl nitrite (78 ml) over 20 min. The mixture was stirred at room temperature for 1 h, then heated at 90° C. and stirred at that temperature for 1 h. After cooling, the mixture was diluted with AcOEt and washed with Na₂S₂O₄ and 1M HC. The organic phases collected were dried and evaporated under vacuum. The residue was purified by silica chromatography eluting with 0-30% AcOEt/petroleum ether to give the ethyl 5-iodo-1H-pyrazole-3-carboxylate (7.5 g) as a yellow solid.

LCMS (ES, m/z): [M+H]⁺: 267

### Synthesis of 8-b: ethyl 5-iodo-1-(4-methoxybenzyl)-1H-pyrazole-3-carboxylate

To a suspension of NaH (1.7 g, 42 mmol, 1.5 equiv, 60%) in DMF (10 ml) was added a solution of ethyl 5-iodo-1H-pyrazole-3-carboxylate (7.5 g, 28 mmol, 1 equiv) in DMF (100 ml) dropwise over 10 min. The resulting solution was stirred for 30 min at room temperature, then a solution of PMB-Cl (5.3 g, 34 mmol, 1.20 equiv) in DMF (10 ml) was added over 10 min. The resulting solution was stirred overnight at room temperature. The reaction mixture was quenched with water/ice (100 ml). The mixture was extracted three times with 200 ml of ether and the organic layers combined and dried over Na₂SO₄. The residue was purified by eluting through a silica gel column with 1:5 EtOAc/PE. This resulted in ethyl 5-iodo-1-(4-methoxybenzyl)-1H-pyrazole-3-carboxylate (6.5 g, 60% yield) as a yellow oil.

LCMS (ES, m/z): [M+H]⁺: 387

### Synthesis of 8-c: ethyl 5-[2-(3-amino-2,6-difluorophenyl)ethynyl]-1-[(4-methoxyphenyl)methyl]pyrazole-3-carboxylate

Into a 10 mL microwave tube was placed ethyl 5-iodo-1-[(4-methoxyphenyl)methyl]pyrazole-3-carboxylate (300 mg, 0.8 mmol, 1 equiv), 3-ethynyl-2,4-difluoroaniline (119 mg, 0.8 mmol, 1 equiv), Pd(dppf)Cl₂ (57 mg, 0.08 mmol, 0.1 equiv), DBU (12 mg, 0.08 mmol, 0.1 equiv), Cs₂CO₃ (253 mg, 0.8 mmol, 1 equiv) and DMF (3 mL). The resulting solution was stirred for 10 min at 150° C. in a microwave reactor. The residue was purified on silica gel eluting with ethyl acetate/petroleum ether (1:2). This gave ethyl 5-[2-(3-amino-2,6-difluorophenyl) ethynyl]-1-[(4-methoxyphenyl)methyl]pyrazole-3-carboxylate (200 mg, 63% yield) as a white solid.

LCMS (ES, m/z): [M+H]⁺: 412

### Synthesis of 8-d: ethyl 5-[2-(3-amino-2,6-difluorophenyl)ethyl]-1-[(4-methoxyphenyl)methyl]pyrazole-3-carboxylate

Into a 25 mL pressure tank reactor, was placed ethyl 5-[2-(3-amino-2,6-difluorophenyl)ethynyl]-1-[(4-methoxyphenyl)methyl]pyrazole-3-carboxylate (200 mg, 0.5 mmol, 1 equiv), MeOH (5 mL), 10% Pd/C (20 mg, 0.1 equiv) and H₂ (2 MPa). The resulting solution was stirred for 12 h at 60° C. in an oil bath. The solids were filtered off and the resulting mixture was concentrated under vacuum to give ethyl 5-[2-(3-amino-2,6-difluorophenyl)ethyl]-1-[(4-methoxyphenyl)methyl]pyrazole-3-carboxylate (171 mg, 85% yield) as a white solid.

LCMS (ES, m/z): [M+H]⁺: 416

### Synthesis of 8-e: ethyl 5-[2-[3-(5-chloro-2-methoxypyridine-3-sulfonamido)-2,6-difluorophenyl]ethyl]-1-[(4-methoxyphenyl)methyl]pyrazole-3-carboxylate

Into a 25 mL 3-necked round-bottom flask, was placed ethyl 5-[2-(3-amino-2,6-difluorophenyl)ethyl]-1-[(4-methoxyphenyl)methyl]pyrazole-3-carboxylate (171 mg, 0.4 mmol, 1 equiv), pyridine (98 mg, 1.2 mmol, 3 equiv), 5-chloro-2-methoxypyridine-3-sulfonyl chloride (100 mg, 0.4 mmol, 1 equiv) and DCM (10 mL). The resulting solution was stirred for 2 h at room temperature. The mixture was concentrated under vacuum and the residue purified on a silica gel column eluting with ethyl acetate/petroleum ether (1:3). Ethyl 5-[2-[3-(5-chloro-2-methoxypyridine-3-sulfonamido)-2,6-difluorophenyl]ethyl]-1-[(4-methoxyphenyl)methyl]pyrazole-3-carboxylate (194 mg, 76% yield) was isolated as a white solid.

LCMS (ES, m/z): [M+H]⁺: 621

### Synthesis of 8-f: 5-[2-[3-(5-chloro-2-methoxypyridine-3-sulfonamido)-2,6-difluorophenyl]ethyl]-1-[(4-methoxyphenyl)methyl]-N-methylpyrazole-3-carboxamide

Into a 25 mL 3-necked round-bottom flask, was placed ethyl 5-[2-[3-(5-chloro-2-methoxypyridine-3-sulfonamido)-2,6-difluorophenyl]ethyl]-1-[(4-methoxyphenyl)methyl]pyrazole-3-carboxylate (194 mg, 0.3 mmol, 1 equiv), and methylamine solution (5 mL, 30% in EtOH). The resulting solution was stirred for 12 h at room temperature. The mixture was concentrated under vacuum to give 5-[2-[3-(5-chloro-2-methoxypyridine-3-sulfonamido)-2,6-difluorophenyl]ethyl]-1-[(4-methoxyphenyl)methyl]-N-methylpyrazole-3-carboxamide (130 mg, 69% yield) as a yellow oil.

LCMS (ES, m/z): [M+H]⁺: 606

### Synthesis of Compound 8: 5-[2-[3-(5-chloro-2-methoxypyridine-3-sulfonamido)-2,6-difluorophenyl]ethyl]-N-methyl-1H-pyrazole-3-carboxamide

5-[2-[3-(5-Chloro-2-methoxypyridine-3-sulfonamido)-2,6-difluorophenyl]ethyl]-1-[(4-methoxyphenyl)methyl]-N-methylpyrazole-3-carboxamide (130 mg, 0.2 mmol, 1 equiv) in trifluoroacetic acid (3 ml) was stirred for 2 h at 70° C. in an oil bath. The resulting mixture was concentrated under vacuum. The crude product (200 mg) was purified by Flash-Prep-HPLC with the following conditions: welch Vltimate XB—C18, 50×250 mm, 10 μm mobile phase, Mobile Phase A: Water (10 mmol/L NH₄HCO₃), Mobile Phase B: ACN (10-60% over 15 min); Detector, 220 nm. 5-[2-[3-(5-Chloro-2-methoxypyridine-3-sulfonamido)-2,6-difluorophenyl]ethyl]-N-methyl-1H-pyrazole-3-carboxamide (43 mg, 41% yield) was isolated as a white solid.

LCMS (ES, m/z): [M+H]⁺: 486
¹H NMR (300 MHz, DMSO-d₆) δ 10.25 (s, 1H), 8.50 (d, J=2.6 Hz, 1H), 8.01 (d, J=2.6 Hz, 1H), 7.96 (s, 1H), 7.14 (td, J=8.7, 5.9 Hz, 1H), 7.07-6.95 (m, 1H), 6.28 (s, 1H), 3.93 (s, 3H), 2.86 (s, 2H), 2.72 (d, J=4.8 Hz, 5H).

### Example 20: Synthesis of N-(3-(2-(1H-pyrazolo[3,4-b]pyridin-5-yl)ethyl)-2,4-difluorophenyl)-5-fluoro-2-methylpyridine-3-sulfonamide (Compound 9)

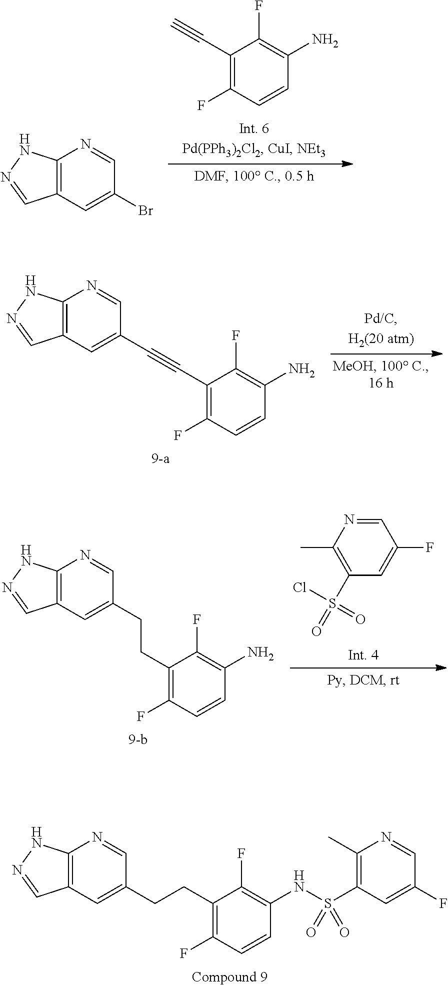 [SMILES: C#Cc1c(F)ccc(N)c1F ; Brc1cnc2[nH]ncc2c1 ; Nc1ccc(F)c(C#Cc2cnc3[nH]ncc3c2)c1F ; Nc1ccc(F)c(CCc2cnc3[nH]ncc3c2)c1F ; Cc1ncc(F)cc1S(=O)(=O)Cl ; Cc1ncc(F)cc1S(=O)(=O)Nc1ccc(F)c(CCc2cnc3[nH]ncc3c2)c1F]

### Synthesis of 9-a: 2,4-difluoro-3-(2-[1H-pyrazolo[3,4-b]pyridin-5-yl]ethynyl)aniline

To a solution of 5-bromo-1H-pyrazolo[3,4-b]pyridine (500 mg, 2.5 mmol, 1 equiv) in DMF (10 mL) was added 3-ethynyl-2,4-difluoroaniline (463 mg, 3.0 mmol, 1.2 equiv), TEA (766 mg, 7.5 mmol, 3 equiv), CuI (48 mg, 0.2 mmol, 0.1 equiv) and Pd(PPh₃)₂Cl₂ (177 mg, 0.2 mmol, 0.1 equiv) at room temperature under a N₂ atmosphere. The resulting mixture was stirred at 100° C. for 0.5 h. After cooling to room temperature, water (20 mL) was added and mixture was extracted with ethyl acetate (3×20 mL). The combined organic extracts were washed with brine (2×10 mL), dried over anhydrous Na₂SO₄, filtered, and concentrated under reduced pressure. The residue was purified by silica gel chromatography (eluting with PE:EA=3:1) to afford 2,4-difluoro-3-(2-[1H-pyrazolo[3,4-b]pyridin-5-yl]ethynyl)aniline (500 mg, 73% yield) as a brown solid.

LCMS (ES, m/z): [M+H]⁺: 271

### Synthesis of 9-b: 2,4-difluoro-3-(2-[1H-pyrazolo[3,4-b]pyridin-5-yl]ethyl)aniline

10% Pd/C (20 mg) was added to a solution of 2,4-difluoro-3-(2-[1H-pyrazolo[3,4-b]pyridin-5-yl]ethynyl)aniline (200 mg, 0.7 mmol, 1 equiv) in MeOH (20 mL) in a pressure tank. The mixture was heated to 100° C. under H₂ (20 atm) for 16 h, and then filtered through a Celite pad and concentrated under reduced pressure. The residue was purified by silica gel chromatography, eluting with PE:EA (2:1) to afford 2,4-difluoro-3-(2-[1H-pyrazolo[3,4-b]pyridin-5-yl]ethyl)aniline (100 mg, 49% yield) as a white solid.

LCMS (ES, m/z): [M+H]⁺: 274

### Synthesis of Compound 9: N-(3-(2-(1H-pyrazolo[3,4-b]pyridin-5-yl)ethyl)-2,4-difluorophenyl)-5-fluoro-2-methylpyridine-3-sulfonamide

Pyridine (86 mg, 1 mmol, 3 equiv) and then 5-fluoro-2-methylpyridine-3-sulfonyl chloride (130 mg, 0.45 mmol, 1.5 equiv) were added to a solution of 2,4-difluoro-3-(2-[1H-pyrazolo[3,4-b]pyridin-5-yl]ethyl)aniline (100 mg, 0.3 mmol, 1 equiv) in DCM (5 mL) at room temperature. The reaction mixture was stirred for 1 h at room temperature and then concentrated under reduced pressure and the crude residue was purified by prep-HPLC with the following conditions: Column: welch Vltimate XB—C18, 50×250 mm, 10 μm; Mobile Phase: 20-50% MeCN/0.1% aqueous formic acid; Detector: 220 nm; to afford N-(3-(2-(1H-pyrazolo[3,4-b]pyridin-5-yl)ethyl)-2,4-difluorophenyl)-5-fluoro-2-methylpyridine-3-sulfonamide (60 mg, 34% yield) as a white solid.

LCMS (ES, m/z): [M+H]⁺: 448
¹H NMR (300 MHz, DMSO-d₆) δ 13.51 (s, 1H), 8.67 (d, J=2.8 Hz, 1H), 8.21 (d, J=2.1 Hz, 1H), 8.03 (s, 1H), 7.90-7.75 (m, 2H), 7.08 (td, J=9.0, 6.0 Hz, 1H), 6.89 (td, J=9.0, 1.7 Hz, 1H), 2.85 (t, J=4.8 Hz, 4H), 2.73 (d, J=1.2 Hz, 3H).

### Example 21: Synthesis of 5-cyano-N-[2,4-difluoro-3-(2-[1H-pyrazolo[3,4-b]pyridin-5-yl]ethyl)phenyl]-2-methoxypyridine-3-sulfonamide (Compound 10)

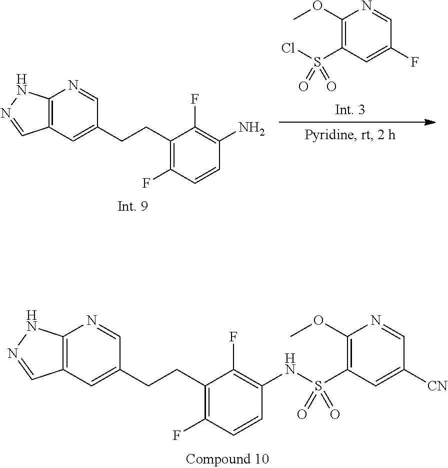 [SMILES: Nc1ccc(F)c(CCc2cnc3[nH]ncc3c2)c1F ; COc1ncc(F)cc1S(=O)(=O)Cl ; COc1ncc(C#N)cc1S(=O)(=O)Nc1ccc(F)c(CCc2cnc3[nH]ncc3c2)c1F]

### Synthesis of Compound 10: 5-cyano-N-[2,4-difluoro-3-(2-[1H-pyrazolo[3,4-b]pyridin-5-yl]ethyl)phenyl]-2-methoxypyridine-3-sulfonamide

To a stirred solution of 2,4-difluoro-3-(2-[1H-pyrazolo[3,4-b]pyridin-5-yl]ethyl)aniline (100 mg, 0.4 mmol, 1 equiv) in DCM (10 mL) was added 5-cyano-2-methoxypyridine-3-sulfonyl chloride (85 mg, 0.4 mmol, 1 equiv) and pyridine (87 mg, 1 mmol, 3 equiv). The resulting solution was stirred for 2 h at room temperature and then concentrated under reduced pressure. The residue was purified by prep-HPLC with the following conditions: Column: welch Vltimate XB—C18, 50×250 mm, 10 μm; Mobile Phase: 10-65% MeCN/0.1% aqueous formic acid; Detector: 220 nm; to afford 5-cyano-N-[2,4-difluoro-3-(2-[1H-pyrazolo[3,4-b]pyridin-5-yl]ethyl)phenyl]-2-methoxypyridine-3-sulfonamide (59 mg, 35% yield) as a white solid.

LCMS (ES, m/z): [M+H]⁺: 471
¹H NMR (300 MHz, DMSO-d₆) δ 13.52 (s, 1H), 10.33 (s, 1H), 8.93 (d, J=2.3 Hz, 1H), 8.42 (d, J=2.3 Hz, 1H), 8.21 (d, J=2.1 Hz, 1H), 8.04 (s, 1H), 7.84 (d, J=2.1 Hz, 1H), 7.12 (td, J=8.9, 5.9 Hz, 1H), 6.94 (t, J=9.0 Hz, 1H), 4.03 (s, 3H), 2.87 (p, J=7.2, 6.0 Hz, 4H).

### Example 22: Synthesis of 5-chloro-N-[2,4-difluoro-3-(2-[1H-pyrazolo[4,3-b]pyridin-5-yl]ethyl)phenyl]-2-methoxypyridine-3-sulfonamide (Compound 11)

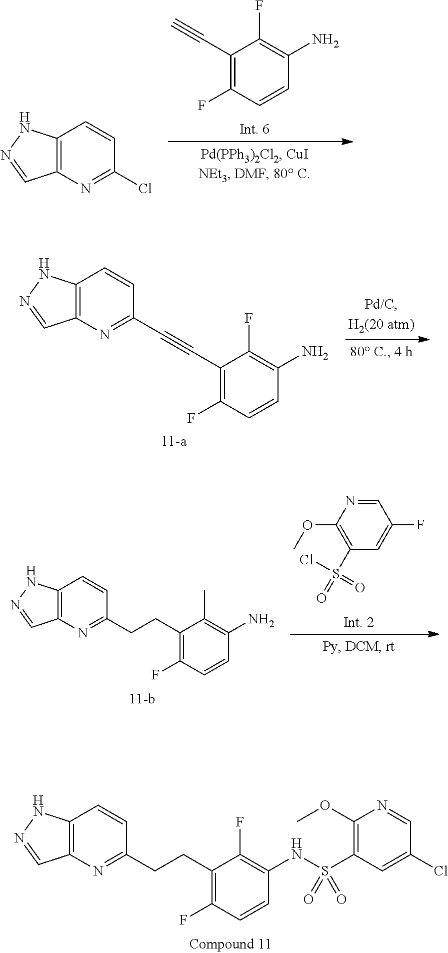 [SMILES: C#Cc1c(F)ccc(N)c1F ; Clc1ccc2[nH]ncc2n1 ; Nc1ccc(F)c(C#Cc2ccc3[nH]ncc3n2)c1F ; Cc1c(N)ccc(F)c1CCc1ccc2[nH]ncc2n1 ; COc1ncc(F)cc1S(=O)(=O)Cl ; COc1ncc(Cl)cc1S(=O)(=O)Nc1ccc(F)c(CCc2ccc3[nH]ncc3n2)c1F]

### Synthesis of 11-a: 2,4-difluoro-3-(2-[1H-pyrazolo[4,3-b]pyridin-5-yl]ethynyl)aniline

CuI (0.25 g, 1.3 mmol, 0.1 equiv) and Pd(PPh₃)₂Cl₂ (0.92 g, 1.3 mmol, 0.1 equiv) were added to a stirred solution of 5-chloro-1H-pyrazolo[4,3-b]pyridine (2 g, 13 mmol, 1 equiv), TEA (3.30 g, 32.6 mmol, 2.5 equiv) and 3-ethynyl-2,4-difluoroaniline (2 g, 13 mmol, 1 equiv) in DMF (20 mL) under a nitrogen atmosphere. After stirring for 0.5 h at 80° C. under nitrogen atmosphere, the reaction mixture was concentrated under reduced pressure. The residue was purified by silica gel chromatography, eluting with PE:EA (1:1-0:1) to afford 2,4-difluoro-3-(2-[1H-pyrazolo[4,3-b]pyridin-5-yl]ethynyl)aniline (2.2 g, 62% yield) as a yellow solid.

LCMS (ES, m/z): [M+H]⁺: 271

### Synthesis of 11-b: 2,4-difluoro-3-(2-[1H-pyrazolo[4,3-b]pyridin-5-yl]ethyl)aniline

10% Pd/C (78 mg) was added to a solution of 2,4-difluoro-3-(2-[1H-pyrazolo[4,3-b]pyridin-5-yl]ethynyl)aniline (200 mg, 0.7 mmol, 1 equiv) in MeOH (20 mL). The mixture was heated to 80° C. for 4 h under H₂ (20 atm). The reaction mixture was cooled to room temperature, filtered through a Celite pad and concentrated under reduced pressure. The residue was purified by silica gel chromatography (eluting with PE:EA=1:1) to afford 2,4-difluoro-3-(2-[1H-pyrazolo[4,3-b]pyridin-5-yl]ethyl)aniline (90 mg, 44% yield) as a yellow oil.

LCMS (ES, m/z): [M+H]⁺: 275

### Synthesis of Compound 11: 5-chloro-N-[2,4-difluoro-3-(2-[1H-pyrazolo[4,3-b]pyridin-5-yl]ethyl)phenyl]-2-methoxypyridine-3-sulfonamide

Pyridine (86 mg, 1.1 mmol, 3 equiv) and 5-chloro-2-methoxypyridine-3-sulfonyl chloride (132 mg, 0.45 mmol, 1.5 equiv) were added to a solution of 2,4-difluoro-3-(2-[1H-pyrazolo[4,3-b]pyridin-5-yl]ethyl)aniline (100 mg, 0.3 mmol, 1 equiv) in DCM (5 mL) at room temperature. The reaction mixture was stirred for 2 hrs and then concentrated under reduced pressure. The crude residue was purified by prep-HPLC with the following conditions: Column: welch Vltimate XB—C18; 50×250 mm; 10 μm; Mobile Phase: 25-50% MeCN/0.1% aqueous formic acid; Detector: 220 nm; to afford 5-chloro-N-[2,4-difluoro-3-(2-[1H-pyrazolo[4,3-b]pyridin-5-yl]ethyl)phenyl]-2-methoxypyridine-3-sulfonamide (40 mg, 23% yield) as a white solid.

LCMS (ES, m/z): [M+H]⁺: 480
¹H NMR (300 MHz, DMSO-d₆) δ 13.20 (s, 1H), 8.47 (d, J=2.6 Hz, 1H), 8.18 (s, 1H), 7.98 (d, J=2.6 Hz, 1H), 7.87 (dd, J=8.6, 1.1 Hz, 1H), 7.11 (dq, J=8.9, 3.2 Hz, 2H), 6.94 (t, J=8.8 Hz, 1H), 3.92 (s, 3H), 2.98 (s, 4H).

### Example 23: Synthesis of 5-chloro-N-[2,4-difluoro-3-(2-[1H-pyrrolo[2,3-b]pyridin-5-yl]ethyl)phenyl]-2-methoxypyridine-3-sulfonamide (Compound 12)

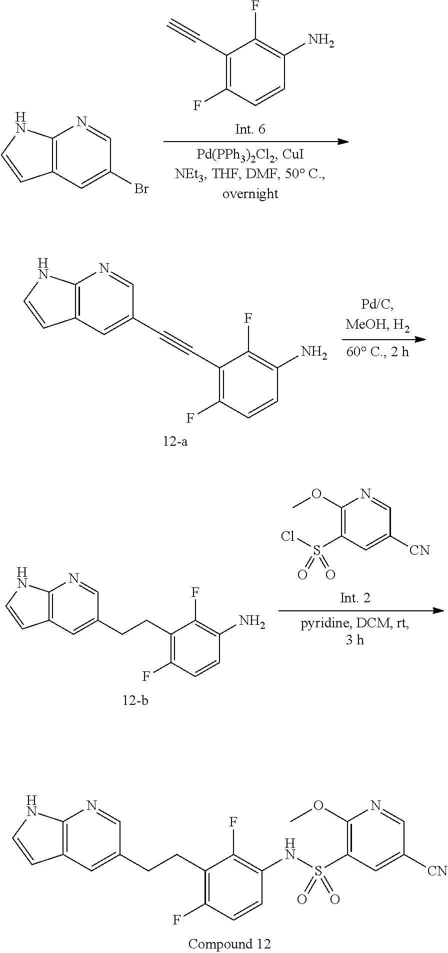 [SMILES: C#Cc1c(F)ccc(N)c1F ; Brc1cnc2[nH]ccc2c1 ; Nc1ccc(F)c(C#Cc2cnc3[nH]ccc3c2)c1F ; COc1ncc(C#N)cc1S(=O)(=O)Cl ; Nc1ccc(F)c(CCc2cnc3[nH]ccc3c2)c1F ; COc1ncc(C#N)cc1S(=O)(=O)Nc1ccc(F)c(CCc2cnc3[nH]ccc3c2)c1F]

### Synthesis of 12-a: 2,4-difluoro-3-(2-[1H-pyrrolo[2,3-b]pyridin-5-yl]ethynyl)aniline

5-Bromo-1H-pyrrolo[2,3-b]pyridine (196 mg, 0.1 mmol, 1 equiv), DMF (2.5 mL), THE (2.5 mL), NEt₃ (302 mg, 3 mmol, 3 equiv), CuI (19 mg, 0.1 mmol, 0.1 equiv), Pd(PPh₃)₂Cl₂ (70 mg, 0.1 mmol, 0.1 equiv) and 3-ethynyl-2,4-difluoroaniline (183 mg, 1.2 mmol, 1.2 equiv) were placed into a 40 mL vial. The reaction mixture was stirred at 50° C. for 16 h and then diluted with water (20 mL). The resulting solution was extracted with EtOAc (2×20 mL) and the combined organic extracts washed water (3×20 mL), dried over anhydrous sodium sulfate and concentrated. The crude residue was purified by Flash-Prep-HPLC with the following conditions: Column: WelFlash TM C18-I; Spherical C18 20-40 m; 120 g; mobile phase 5-80% MeCN/0.1% aqueous formic acid; Detector 220 nm; to afford 2,4-difluoro-3-(2-[1H-pyrrolo[2,3-b]pyridin-5-yl]ethynyl) aniline (105 mg, 39% yield) as a yellow solid.

LCMS (ES, m/z): [M+H]⁺: 270

### Synthesis of 12-b: 2,4-difluoro-3-(2-[1H-pyrrolo[2,3-b]pyridin-5-yl]ethyl)aniline

10% Pd/C (45 mg, 0.4 mmol, 1.3 equiv) was added to a solution of 2,4-difluoro-3-(2-[1H-pyrrolo[2,3-b]pyridin-5-yl]ethynyl)aniline (90 mg, 0.3 mmol, 1 equiv) in MeOH (5 mL) in a 50 mL sealed tube. The suspension was heated to 60° C. for 2 h under H₂ (20 atm). The reaction mixture was cooled to room temperature, filtered, and concentrated under reduced pressure to afford 2,4-difluoro-3-(2-[1H-pyrrolo[2,3-b]pyridin-5-yl]ethyl)aniline (80 mg, 88% yield) as a white solid which was used directly in next step without further purification.

LCMS (ES, m/z): [M+H]⁺: 274

### Synthesis of Compound 12: 5-chloro-N-[2,4-difluoro-3-(2-[1H-pyrrolo[2,3-b]pyridin-5-yl]ethyl)phenyl]-2-methoxypyridine-3-sulfonamide

Pyridine (230 mg, 3.0 mmol, 10 equiv) and then 5-chloro-2-methoxypyridine-3-sulfonyl chloride (99 mg, 0.4 mmol, 1.4 equiv) were added to a solution of 2,4-difluoro-3-(2-[1H-pyrrolo[2,3-b]pyridin-5-yl]ethyl)aniline (80 mg, 0.3 mmol, 1 equiv) in DCM (3 mL) and the reaction mixture stirred for 3 hours at room temperature. The reaction mixture was concentrated and the crude residue purified by Prep-HPLC with the following conditions: Column: welch Vltimate XB—C18, 50×250 mm, 10 μm; Mobile Phase: 42-65% MeCN/0.1% aqueous formic acid; Detector: 220 nm; to afford 5-chloro-N-[2,4-difluoro-3-(2-[1H-pyrrolo[2,3-b]pyridin-5-yl]ethyl)phenyl]-2-methoxypyridine-3-sulfonamide (41 mg, 29% yield) as a white solid.

LCMS (ES, m/z): [M+H]⁺: 479
¹H NMR (300 MHz, DMSO-d₆) δ 11.48 (s, 1H), 8.48 (d, J=2.6 Hz, 1H), 8.17 (s, 1H), 7.99 (d, J=2.6 Hz, 1H), 7.90 (d, J=2.1 Hz, 1H), 7.61 (d, J=2.0 Hz, 1H), 7.41 (dd, J=3.4, 2.5 Hz, 1H), 7.10 (td, J=8.9, 5.9 Hz, 1H), 6.91 (td, J=9.1, 1.6 Hz, 1H), 6.34 (dd, J=3.4, 1.8 Hz, 1H), 3.94 (s, 3H), 2.86 (m, 2H), 2.80 (m, 2H).

### Example 24: Synthesis of 5-chloro-N-[2,4-difluoro-3-(2-[1H-pyrazolo[3,4-c]pyridin-5-yl]ethyl)phenyl]-2-methoxypyridine-3-sulfonamide (Compound 13)

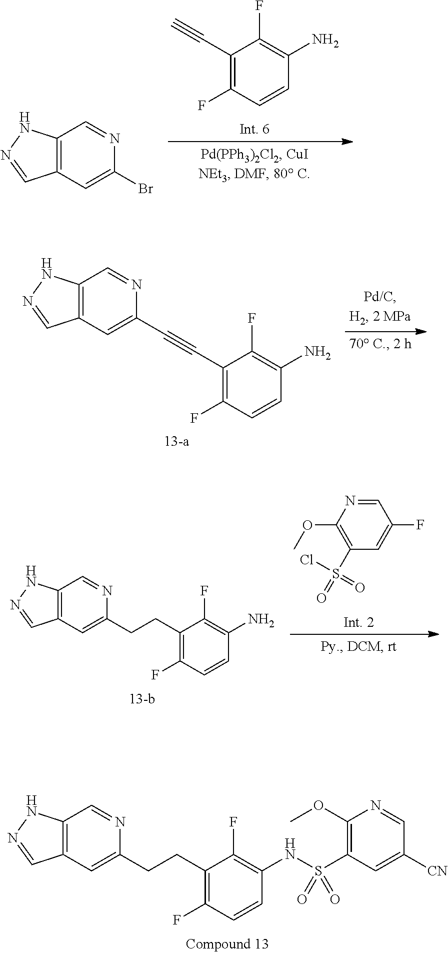 [SMILES: C#Cc1c(F)ccc(N)c1F ; Brc1cc2cn[nH]c2cn1 ; Nc1ccc(F)c(C#Cc2cc3cn[nH]c3cn2)c1F ; Nc1ccc(F)c(CCc2cc3cn[nH]c3cn2)c1F ; COc1ncc(F)cc1S(=O)(=O)Cl ; COc1ncc(C#N)cc1S(=O)(=O)Nc1ccc(F)c(CCc2cc3cn[nH]c3cn2)c1F]

### Synthesis of 13-a: 3-((1H-pyrazolo[3,4-c]pyridin-5-yl)ethynyl)-2,4-difluoroaniline

3-Ethynyl-2,4-difluoroaniline (770 mg, 5.1 mmol, 1 equiv), TEA (1.5 g, 15 mmol, 3 equiv), CuI (96 mg, 0.5 mmol, 0.1 equiv) and Pd(PPh₃)₂Cl₂ (350 mg, 0.5 mmol, 0.1 equiv) were added to a solution of 5-bromo-1H-pyrazolo[3,4-c]pyridine (1.0 g, 5 mmol, 1 equiv) in DMF (10 mL) under an atmosphere of nitrogen. The reaction mixture was heated to 80° C. for 16 hours. After cooling to room temperature, water (200 mL) was added and the mixture extracted with ethyl acetate (3×200 mL). The combined organic extracts were washed with brine, dried over Na₂SO₄, filtered, and concentrated under reduced pressure. The crude residue was purified by silica gel chromatography, eluting with PE:EA=1:1, to afford 3-((1H-pyrazolo[3,4-c]pyridin-5-yl)ethynyl)-2,4-difluoroaniline (1.0 g, 73% yield) as a brown solid.

LCMS (ES, m/z): [M+H]⁺: 271

### Synthesis of 13-b: 2,4-difluoro-3-(2-[1H-pyrazolo[3,4-c]pyridin-5-yl]ethyl)aniline

10% Pd/C (78 mg) was added to a solution of 2,4-difluoro-3-(2-[1H-pyrazolo[3,4-c]pyridin-5-yl]ethynyl)aniline (200 mg, 0.7 mmol, 1 equiv) in MeOH (20 mL) and THE (3 mL). The suspension was stirred at 70° C. for 2 h under a H₂ atmosphere (2 MPa). The mixture was filtered through Celite, concentrated under reduced pressure, and the residue purified by silica gel chromatography, eluting with PE:EA=1:1, to afford product 2,4-difluoro-3-(2-[1H-pyrazolo[3,4-c]pyridin-5-yl]ethyl)aniline (100 mg, 49% yield) as a yellow oil.

LCMS (ES, m/z): [M+H]⁺: 275

### Synthesis of Compound 13: 5-chloro-N-[2,4-difluoro-3-(2-[1H-pyrazolo[3,4-c]pyridin-5-yl]ethyl)phenyl]-2-methoxypyridine-3-sulfonamide

Pyridine (52 mg, 0.6 mmol, 3 equiv) and 5-chloro-2-methoxypyridine-3-sulfonyl chloride (80 mg, 0.3 mmol, 1.5 equiv) were added to a solution of 2,4-difluoro-3-(2-[1H-pyrazolo[3,4-c]pyridin-5-yl]ethyl)aniline (60 mg, 0.2 mmol, 1 equiv) in DCM (5 mL) and the reaction mixture stirred at room temperature for 1 h. The mixture was concentrated under reduced pressure and the residue was purified by prep-HPLC with the following conditions: Column: welch Vltimate XB—C18; 50×250 mm; 10 μm mobile phase; Mobile Phase: 25-40% MeCN/0.1% aqueous formic acid; Detector: 220 nm; to afford 5-chloro-N-[2,4-difluoro-3-(2-[1H-pyrazolo[3,4-c]pyridin-5-yl]ethyl)phenyl]-2-methoxypyridine-3-sulfonamide (15 mg, 14% yield) as an off-white solid.

LCMS (ES, m/z): [M+H]⁺: 480
¹H NMR (300 MHz, DMSO-d₆) δ 13.50 (s, 1H), 8.96 (s, 1H), 8.38 (d, J=2.5 Hz, 1H), 8.10 (s, 1H), 7.96 (d, J=2.6 Hz, 1H), 7.43 (s, 1H), 7.05 (q, J=8.4, 7.7 Hz, 1H), 6.83 (t, J=9.1 Hz, 1H), 3.89 (s, 3H), 2.95 (s, 4H).

### Example 25: Synthesis of 5-chloro-N-[2,4-difluoro-3-[([3-methyl-1H-pyrazolo[3,4-b]pyridin-5-yl]oxy)methyl]phenyl]-2-methoxypyridine-3-sulfonamide (Compound 14)

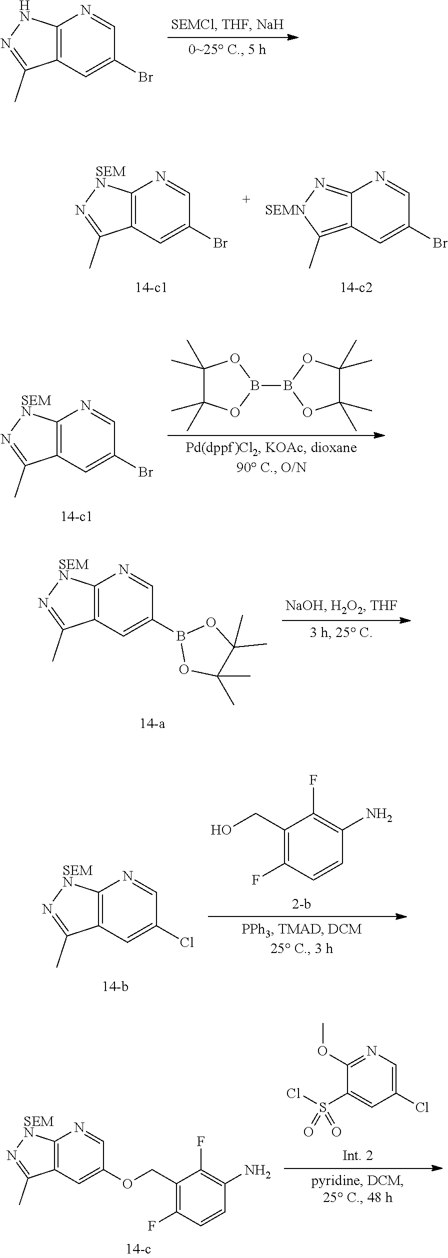

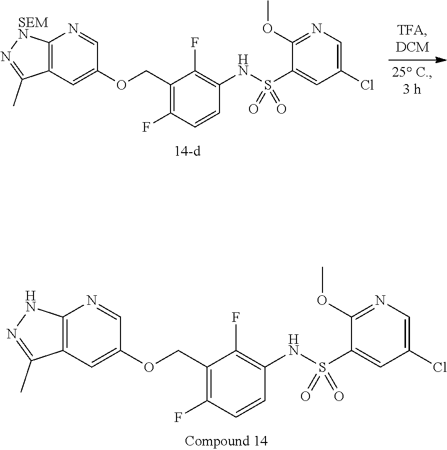 [SMILES: COc1ncc(Cl)cc1S(=O)(=O)Nc1ccc(F)c(COc2cnc3c(c2)c(C)nn3COCC[Si](C)(C)C)c1F ; COc1ncc(Cl)cc1S(=O)(=O)Nc1ccc(F)c(COc2cnc3[nH]nc(C)c3c2)c1F]

### Synthesis of 14-c1 & 14-c2: 5-bromo-3-methyl-1-[[2-(trimethylsilyl)ethoxy]methyl]pyrazolo[3,4-b]pyridine and 5-bromo-3-methyl-2-((2-(trimethylsilyl)ethoxy)methyl)-2H-pyrazolo[3,4-b]pyridine

NaH (0.23 g, 9.5 mmol, 2 equiv) was added portion wise to a stirred solution of 5-bromo-3-methyl-1H-pyrazolo[3,4-b]pyridine (1 g, 4.7 mmol, 1 equiv) and [2-(chloromethoxy)ethyl]trimethylsilane (1.6 g, 9.4 mmol, 2 equiv) in THE (30 mL) at 0° C. The reaction mixture was stirred at room temperature for 5 hours and then quenched with water (50 mL). The mixture was extracted with EA (3×30 mL) and the combined organic extracts washed with brine (50 mL), dried over anhydrous sodium sulfate, and concentrated under reduced pressure. The crude residue was purified by silica gel chromatography, eluting with PE:EA (5:1-2:1) to afford 5-bromo-3-methyl-1-[[2-(trimethylsilyl)ethoxy]methyl]pyrazolo[3,4-b]pyridine (0.8 g, 50% yield) as colorless oil and 5-bromo-3-methyl-2-((2-(trimethylsilyl)ethoxy)methyl)-2H-pyrazolo[3,4-b]pyridine (0.64 g, 37% yield).

LCMS (ES, m/z): [M+H]⁺: 342

### Synthesis of 14-a: 3-methyl-5-(4,4,5,5-tetramethyl-1,3,2-dioxaborolan-2-yl)-1-[[2-(trimethylsilyl)ethoxy]methyl]pyrazolo[3,4-b]pyridine

5-Bromo-3-methyl-1-[[2-(trimethylsilyl)ethoxy]methyl]pyrazolo[3,4-b]pyridine (0.8 g, 2.3 mmol, 1 equiv), bis(pinacolato)diboron (1.2 g, 4.7 mmol, 2 equiv), KOAc (0.46 g, 4.7 mmol, 2 equiv), Pd(dppf)Cl₂ (0.32 g, 0.45 mmol, 0.2 equiv) were suspended in dioxane (12 mL) under an atmosphere of nitrogen and stirred at 90° C. for 16 h. The reaction mixture was cooled to room temperature, diluted with water (50 mL), and extracted with EA (2×30 mL). The combined organic extracts were washed with water (50 mL), dried over anhydrous sodium sulfate, filtered, and concentrated under reduced pressure. The residue was purified by silica gel column chromatography, eluting with PE:EA (3:1), to afford 3-methyl-5-(4,4,5,5-tetramethyl-1,3,2-dioxaborolan-2-yl)-1-[[2-(trimethylsilyl)ethoxy]methyl]pyrazolo[3,4-b]pyridine (1.6 g, 172%, 60% purity) as colorless oil.

LCMS (ES, m/z): [M+H]⁺: 390

### Synthesis of 14-b: 3-methyl-1-[[2-(trimethylsilyl)ethoxy]methyl]pyrazolo[3,4-b]pyridin-5-ol

NaOH (1 M, 16 mL, 16 mmol) and aqueous H₂O₂ (30%, 1.5 g, 14 mmol, 6 equiv) were added to a solution of 3-methyl-5-(4,4,5,5-tetramethyl-1,3,2-dioxaborolan-2-yl)-1-[[2-(trimethylsilyl)ethoxy]methyl]pyrazolo[3,4-b]pyridine (1.6 g, 2.5 mmol, 1 equiv) in THE (16 mL). The resulting solution was stirred for 3 h at 25° C. and then diluted with water (50 mL). The mixture was extracted with EA (2×30 mL) and the combined organic extracts washed with brine (50 mL), dried over anhydrous sodium sulfate, filtered, and concentrated under reduced pressure. The residue was purified by silica gel chromatography, eluting with PE:EA (2:1) to afford 3-methyl-1-[[2-(trimethylsilyl)ethoxy]methyl]pyrazolo[3,4-b]pyridin-5-ol (0.25 g, 37% yield) as a white solid.

LCMS (ES, m/z): [M+H]⁺: 280

### Synthesis of 14-c: 2,4-difluoro-3-[[(3-methyl-1-[[2-(trimethylsilyl)ethoxy]methyl]pyrazolo[3,4-b]pyridin-5-yl)oxy]methyl]aniline

PPh₃ (300 mg, 1.1 mmol, 1.5 equiv) and TMAD (190 mg, 1.1 mmol, 1.5 equiv) were added to a solution of 3-methyl-1-[[2-(trimethylsilyl)ethoxy]methyl]pyrazolo[3,4-b]pyridin-5-ol (210 mg, 0.8 mmol, 1 equiv) and (3-amino-2,6-difluorophenyl)methanol (130 mg, 0.8 mmol, 1.1 equiv) in DCM (10 mL) under an atmosphere of nitrogen. The reaction mixture was stirred for 3 h at 25° C. and then concentrated under reduced pressure. The residue was purified by silica gel column chromatography, eluting with PE:EA (3:1) to afford 2,4-difluoro-3-[[(3-methyl-1-[[2-(trimethylsilyl)ethoxy]methyl]pyrazolo[3,4-b]pyridin-5-yl)oxy]methyl]aniline (316 mg, 100% yield) as a white solid.

LCMS (ES, m/z): [M+H]⁺: 421

### Synthesis of 14-d: 5-chloro-N-(2,4-difluoro-3-[[(3-methyl-1-[[2-(trimethylsilyl)ethoxy]methyl]pyrazolo[3,4-b]pyridin-5-yl)oxy]methyl]phenyl)-2-methoxypyridine-3-sulfonamide

Pyridine (98 mg, 1.2 mmol, 4 equiv) and 5-chloro-2-methoxypyridine-3-sulfonyl chloride (135 mg, 0.6 mmol, 1.8 equiv) were added to a solution of 2,4-difluoro-3-[[(3-methyl-1-[[2-(trimethylsilyl)ethoxy]methyl]pyrazolo[3,4-b]pyridin-5-yl)oxy]methyl]aniline (130 mg, 0.3 mmol, 1 equiv) in DCM (8 mL) and the reaction mixture was stirred for 48 h at 25° C. The resulting mixture was concentrated under reduced pressure and the residue purified by prep-HPLC with the following conditions: Column: WelFlash TM C18-I; Spherical C18 20-40 μm; 120 g; Mobile Phase: 10-80% MeCN/0.1% aqueous formic acid; Detector 220 nm; to afford 5-chloro-N-(2,4-difluoro-3-[[(3-methyl-1-[[2-(trimethylsilyl)ethoxy]methyl]pyrazolo[3,4-b]pyridin-5-yl)oxy]methyl]phenyl)-2-methoxypyridine-3-sulfonamide (90 mg, 47% yield) as a white solid.

LCMS (ES, m/z): [M+H]⁺: 626

### Synthesis of Compound 14: 5-chloro-N-[2,4-difluoro-3-[([3-methyl-1H-pyrazolo[3,4-b]pyridin-5-yl]oxy)methyl]phenyl]-2-methoxypyridine-3-sulfonamide

A solution of 5-chloro-N-(2,4-difluoro-3-[[(3-methyl-1-[[2-(trimethylsilyl)ethoxy]methyl]pyrazolo[3,4-b]pyridin-5-yl)oxy]methyl]phenyl)-2-methoxypyridine-3-sulfonamide (90 mg, 0.14 mmol, 1 equiv) in TFA (0.8 mL) and DCM (5 mL) was stirred at room temperature for 3 h and then concentrated under reduced pressure. The crude product was purified by Prep-HPLC with the following conditions: Column: welch Vltimate XB—C18, 50×250 mm, 10 μm; Mobile Phase: 20-60% MeCN/0.1% aqueous formic acid; Detector: 220 nm; to afford 5-chloro-N-[2,4-difluoro-3-[([3-methyl-1H-pyrazolo[3,4-b]pyridin-5-yl]oxy)methyl]phenyl]-2-methoxypyridine-3-sulfonamide (44 mg, 64% yield) as a white solid.

LCMS (ES, m/z): [M+H]⁺: 496
¹H NMR (300 MHz, DMSO-d₆) δ 13.09 (s, 1H), 10.38 (s, 1H), 8.46 (d, J=2.6 Hz, 1H), 8.18 (d, J=2.7 Hz, 1H), 8.03 (d, J=2.6 Hz, 1H), 7.86 (d, J=2.7 Hz, 1H), 7.37 (q, J=8.5, 8.1 Hz, 1H), 7.14 (t, J=8.9 Hz, 1H), 5.13 (s, 2H), 3.91 (s, 3H), 2.47 (s, 3H).

### Example 26: Synthesis of N-[2,4-difluoro-3-(2-[1H-pyrazolo[3,4-b]pyridin-5-yl]ethyl)phenyl]-5-fluoro-2-methoxypyridine-3-sulfonamide (Compound 15)

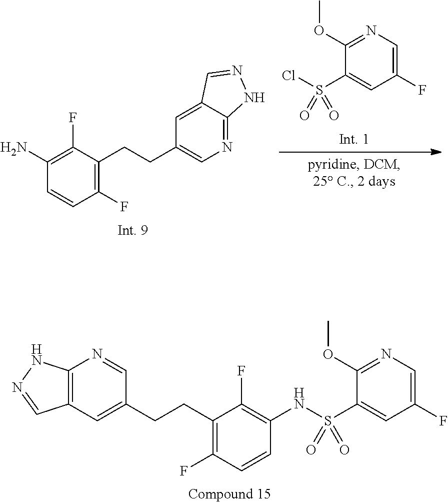 [SMILES: Nc1ccc(F)c(CCc2cnc3[nH]ncc3c2)c1F ; COc1ncc(F)cc1S(=O)(=O)Cl ; COc1ncc(F)cc1S(=O)(=O)Nc1ccc(F)c(CCc2cnc3[nH]ncc3c2)c1F]

### Synthesis of Compound 15: N-[2,4-difluoro-3-(2-[1H-pyrazolo[3,4-b]pyridin-5-yl]ethyl)phenyl]-5-fluoro-2-methoxypyridine-3-sulfonamide

Pyridine (93 mg, 1.2 mmol, 4 equiv) and 5-fluoro-2-methoxypyridine-3-sulfonyl chloride (99 mg, 0.4 mmol, 1.5 equiv) were added to a solution of 2,4-difluoro-3-(2-[1H-pyrazolo[3,4-b]pyridin-5-yl]ethyl)aniline (80 mg, 0.3 mmol, 1 equiv) in DCM (5 mL) and the reaction mixture stirred for 2 days at 25° C. The reaction mixture was concentrated and the residue purified by prep-HPLC with the following conditions: Column: welch Vltimate XB—C18, 50×250 mm, 10 μm; Mobile Phase: 20-60% MeCN/0.1% aqueous formic acid; Detector: 220 nm; to afford N-[2,4-difluoro-3-(2-[1H-pyrazolo[3,4-b]pyridin-5-yl]ethyl)phenyl]-5-fluoro-2-methoxypyridine-3-sulfonamide (38 mg, 28% yield) as a white solid.

LCMS (ES, m/z): [M+H]⁺: 464
¹H NMR (300 MHz, DMSO-d₆) δ 13.51 (s, 1H), 10.22 (s, 1H), 8.45 (d, J=3.0 Hz, 1H), 8.19 (s, 1H), 8.02 (s, 1H), 7.93 (dd, J=7.4, 3.1 Hz, 1H), 7.82 (s, 1H), 7.09 (s, 1H), 6.92 (t, J=9.1 Hz, 1H), 3.93 (s, 3H), 2.88 (s, 4H).

### Example 27: Synthesis of 3-cyano-N-[2,4-difluoro-3-(2-[1H-pyrazolo[3,4-b]pyridin-5-yl]ethyl)phenyl]-5-(trifluoromethyl)benzenesulfonamide (Compound 16)

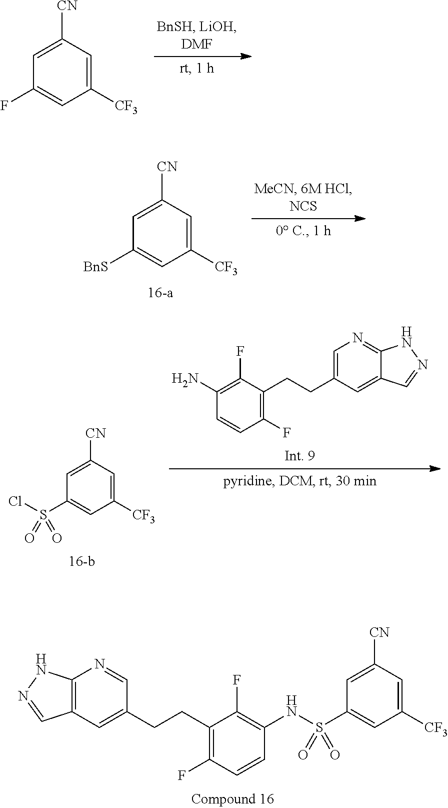

### Synthesis of 16-a: 3-(benzylsulfanyl)-5-(trifluoromethyl)benzonitrile

A solution of 3-fluoro-5-(trifluoromethyl)benzonitrile (2.0 g, 10 mmol, 1 equiv), LiOH (0.5 g, 21 mmol, 2 equiv) and benzyl mercaptan (1.6 g, 13 mmol, 1.2 equiv) in DMF (50 mL) was stirred for 1 h at room temperature. The reaction mixture was diluted with water (100 mL) and then extracted with EA (2×100 mL). The combined organic extracts were washed with water (3×100 mL), dried over anhydrous sodium sulfate, filtered, and concentrated. This afforded 3-(benzylsulfanyl)-5-(trifluoromethyl)benzonitrile (3.0 g, 97% yield) as a yellow oil, which was used directly in next step without further purification.

### Synthesis of 16-b: 3-cyano-5-(trifluoromethyl)benzenesulfonyl chloride

NCS (910 mg, 6.8 mmol, 4 equiv) was added portion wise to a mixture of MeCN (7.5 mL) and aqueous HCl (6 M, 1.5 mL) at 0° C. To this was added 3-(benzylsulfanyl)-5-(trifluoromethyl)benzonitrile (500 mg, 1.7 mmol, 1 equiv) portion wise at 0° C. and the reaction mixture stirred at 0° C. for 1 hour. The reaction mixture was quenched with ice water (20 mL) and extracted with DCM (2×20 mL). The combined organic extracts were washed with water (2×20 mL), dried over anhydrous sodium sulfate, filtered, and concentrated under reduced pressure to afford of 3-cyano-5-(trifluoromethyl)benzenesulfonyl chloride (700 mg, 152% yield, 60% purity) as yellow oil which was used directly in next step without further purification.

### Synthesis of Compound 16: 3-cyano-N-[2,4-difluoro-3-(2-[1H-pyrazolo[3,4-b]pyridin-5-yl]ethyl)phenyl]-5-(trifluoromethyl)benzenesulfonamide

Pyridine (170 mg, 2.2 mmol, 5 equiv) and 3-cyano-5-(trifluoromethyl)benzenesulfonyl chloride (350 mg, 1.3 mmol, 3 equiv) were added to a solution of 2,4-difluoro-3-(2-[1H-pyrazolo[3,4-b]pyridin-5-yl]ethyl)aniline (120 mg, 0.4 mmol, 1 equiv) in DCM (2 mL) and the reaction mixture stirred at room temperature for 30 minutes. The reaction mixture was concentrated and the residue purified by Prep-HPLC with the following conditions: Column: welch Vltimate XB—C18, 50×250 mm, 10 μm; Mobile Phase: 33-53% MeCN/0.1% aqueous formic acid; Detector: 220 nm; to afford 3-cyano-N-[2,4-difluoro-3-(2-[1H-pyrazolo[3,4-b]pyridin-5-yl]ethyl)phenyl]-5-(trifluoromethyl)benzenesulfonamide (85 mg, 38% yield) as a white solid.

LCMS (ES, m/z): [M+H]⁺: 508
¹H NMR (300 MHz, DMSO-d₆) δ 13.51 (s, 1H), 10.51 (s, 1H), 8.75 (s, 1H), 8.42 (d, J=1.7 Hz, 1H), 8.24-8.15 (m, 2H), 8.03 (d, J=1.3 Hz, 1H), 7.86 (d, J=2.1 Hz, 1H), 7.10 (td, J=8.8, 5.9 Hz, 1H), 7.03-6.92 (m, 1H), 2.93-2.81 (m, 4H).

### Example 28: Synthesis of 5-chloro-N-[2,4-difluoro-3-(2-[2-methylpyrazolo[4,3-b]pyridin-6-yl]ethyl)phenyl]-2-methoxypyridine-3-sulfonamide (Compound 17)

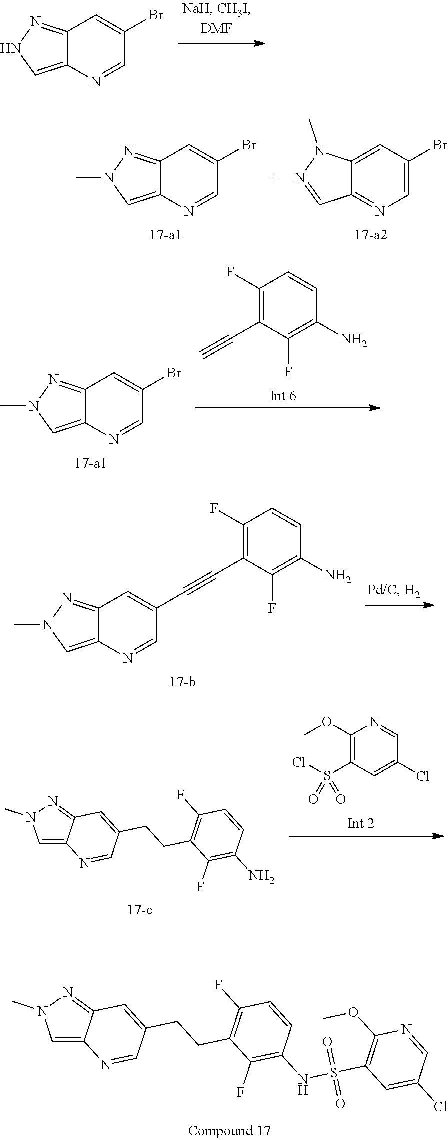

### Synthesis of 17-a: 6-bromo-2-methylpyrazolo[4,3-b]pyridine and 6-bromo-1-methyl-1H-pyrazolo[4,3-b]pyridine

NaH (60% in mineral oil, 218 mg, 9.1 mmol, 2 equiv) was added to a solution of 6-bromo-2H-pyrazolo[4,3-b]pyridine (900 mg, 4.5 mmol, 1 equiv) and methyl iodide (774 mg, 5.4 mmol, 1.2 equiv) in DMF (20 mL) and the reaction mixture stirred at room temperature for 3 hours. The reaction was quenched with water (50 mL) and then extracted EA (3×30 mL). The organic layer was washed with 50 mL of brine. The mixture was dried over anhydrous sodium sulfate and concentrated under vacuum. The crude product was purified by Prep-HPLC with the following conditions: Column: WelFlash TM C18-I; Spherical C18 20-m; 120 g; Mobile Phase: 5-55% MeCN/0.1% aqueous formic acid; Detector 220 nm; to afford 6-bromo-2-methylpyrazolo[4,3-b]pyridine (210 mg, 22% yield) as a white solid and 6-bromo-1-methyl-1H-pyrazolo[4,3-b]pyridine (150 mg, 15% yield) as a white solid. LC-MS (ES, m/z): [M+H]⁺=212

### Synthesis of 17-b: 2,4-difluoro-3-[2-(2-methylindazol-6-yl)ethynyl]aniline

A solution of 6-bromo-2-methylindazole (180 mg, 0.9 mmol, 1 equiv), CuI (17 mg, 0.09 mmol, 0.1 equiv 3-ethynyl-2,4-difluoroaniline (131 mg), TEA (259 mg, 2.6 mmol, 3 equiv) and Pd(PPh₃)₂Cl₂ (60 mg, 0.09 mmol, 0.1 equiv) in DMF (10 mL) under a nitrogen atmosphere was heated to 80° C. for 2 h. The reaction was then quenched by the addition of water (50 mL). The solids were collected by filtration and dried to give 2,4-difluoro-3-[2-(2-methylindazol-6-yl)ethynyl]aniline (260 mg, 110% yield, 80% purity) as a light yellow solid. LC-MS (ES, m/z): [M+H]⁺=285

### Synthesis of 17-c: 5-chloro-N-[2,4-difluoro-3-(2-[2-methylpyrazolo[4,3-b]pyridin-5-yl]ethynyl)phenyl]-2-methoxypyridine-3-sulfonamide

5-Chloro-N-[2,4-difluoro-3-(2-[2-methylpyrazolo[4,3-b]pyridin-6-yl]ethynyl)phenyl]-2-methoxypyridine-3-sulfonamide (100 mg, 0.2 mmol, 1 equiv) and 10% Pd/C (30 mg) in MeOH (10 mL) were stirred at room temperature for 1 h under a H₂ atmosphere (1 atm). The reaction mixture was filtered and the filtrate concentrated under reduced pressure. The crude residue was purified by Prep-HPLC with the following conditions: Column: WelFlash TM C18-I; Spherical C18 20-40 μm; 120 g; mobile phase 10-70% MeCN/0.1% aqueous formic acid Detector 220 nm; to afford 5-chloro-N-[2,4-difluoro-3-(2-[2-methylpyrazolo[4,3-b]pyridin-6-yl]ethyl)phenyl]-2-methoxypyridine-3-sulfonamide (14 mg, 14% yield) as a white solid.

LC-MS (ES, m/z): [M+H]⁺=289

### Synthesis of Compound 17: 5-chloro-N-[2,4-difluoro-3-(2-[2-methylpyrazolo[4,3-b]pyridin-6-yl]ethyl)phenyl]-2-methoxypyridine-3-sulfonamide

Pyridine (150 mg, 1.9 mmol, 3 equiv) and then 5-chloro-2-methoxypyridine-3-sulfonyl chloride (150 mg, 0.6 mmol, 1 equiv) were added to a solution of 2,4-difluoro-3-(2-[2-methylpyrazolo[4,3-b]pyridin-5-yl]ethynyl)aniline (180 mg, 0.6 mmol, 1 equiv) in DCM (8 mL). The reaction mixture was stirred at 30° C. for 16 hours, then concentrated and the residue purified by Prep-HPLC with the following conditions: Column: welch Vltimate XB—C18, 50×25 0 mm, 10 μm; Mobile Phase: 35-65% MeCN/0.1% aqueous formic acid; Detector: 220 nm; to afford 5-chloro-N-[2,4-difluoro-3-(2-[2-methylpyrazolo[4,3-b]pyridin-6-yl]ethyl)phenyl]-2-methoxypyridine-3-sulfonamide (100 mg, 32% yield) as a white solid.

LC-MS (ES, m/z): [M+H]494

### H NMR (300 MHz, DMSO-d₆) δ 8.54 (s, 1H), 8.45 (d, J=2.6 Hz, 1H), 8.22 (s, 1H), 7.98 (d, J=2.6 Hz, 1H), 7.63 (s, 1H), 7.14-7.00 (m, 1H), 6.87 (t, J=9.0 Hz, 1H), 4.18 (s, 3H), 3.92 (s, 3H), 2.95-2.84 (m, 4H).

### Example 29: Synthesis of 5-chloro-N-[2,4-difluoro-3-(2-[1-methylpyrazolo[4,3-b]pyridin-6-yl]ethyl)phenyl]-2-methoxypyridine-3-sulfonamide (Compound 18)

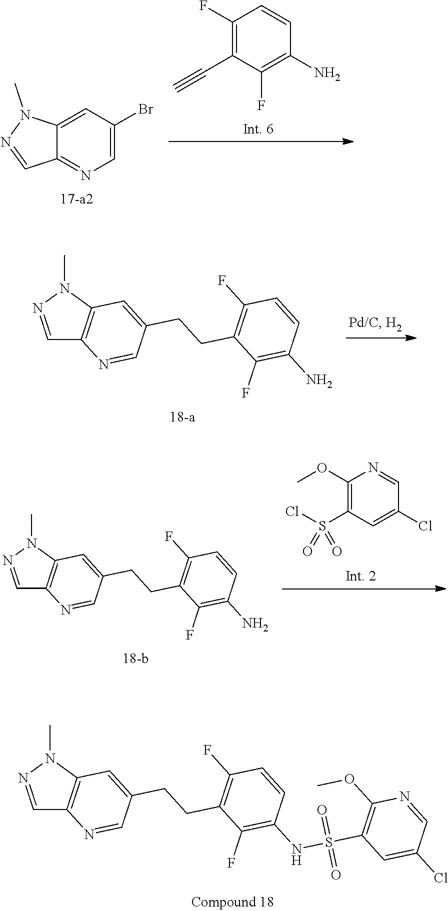

### Synthesis of 18-a: 2,4-difluoro-3-(2-[1-methylpyrazolo[4,3-b]pyridin-6-yl]ethynyl)aniline

A solution of 6-bromo-1-methylindazole (360 mg, 1.7 mmol, 1 equiv), 3-ethynyl-2,4-difluoroaniline (261 mg, 1.7 mmol, 1 equiv), TEA (518 mg, 5.1 mmol, 3 equiv), Pd(PPh₃)₂Cl₂ (120 mg, 0.17 mmol, 0.1 equiv), and CuI (33 mg, 0.17 mmol, 0.1 equiv) in DMF (15 mL) under N₂ atmosphere was stirred at 80° C. for 1 h. The reaction mixture was then quenched by the addition of water and the mixture filtered. The filter cake was dried and afforded 2,4-difluoro-3-(2-[1-methylpyrazolo[4,3-b]pyridin-6-yl]ethynyl)aniline (490 mg, crude) as a light yellow solid.

LC-MS (ES, m/z): [M+H]⁺285

### Synthesis of 18-b: 2,4-difluoro-3-(2-[1-methylpyrazolo[4,3-b]pyridin-6-yl]ethyl)aniline

A suspension of 2,4-difluoro-3-(2-[1-methylpyrazolo[4,3-b]pyridin-6-yl]ethynyl)aniline (100 mg, 0.35 mmol, 1 equiv), and 10% Pd/C (100 mg, 0.94 mmol, 2.7 equiv) in MeOH (5 mL) was stirred at 60° C. for 1 h under H₂ (30 atm). The mixture was filtered and the filtrate concentrated under reduced pressure. The residue was purified by Prep-HPLC with the following conditions: Column: WelFlash TM C18-I, Spherical C18 20-40 μm, 120 g; mobile phase 25-68% MeCN/0.1% aqueous formic acid; Detector 220 nm; to afford 2,4-difluoro-3-(2-[1-methylpyrazolo[4,3-b]pyridin-6-yl]ethyl)aniline (20 mg, 20% yield) as a white solid.

LC-MS (ES, m/z): [M+H]⁺289

### Synthesis of Compound 18: 5-chloro-N-[2,4-difluoro-3-(2-[1-methylpyrazolo[4,3-b]pyridin-6-yl]ethyl)phenyl]-2-methoxypyridine-3-sulfonamide

Pyridine (9 mg, 0.1 mmol, 3 equiv) and 5-chloro-2-methoxypyridine-3-sulfonyl chloride (13 mg, 0.05 mmol, 1.5 equiv) were added to a solution of 2,4-difluoro-3-(2-[1-methylpyrazolo[4,3-b]pyridin-6-yl]ethyl)aniline (10 mg, 0.035 mmol, 1 equiv) in DCM (1 mL) and the reaction mixture stirred at 30° C. for 16 hours. The reaction mixture was concentrated under reduced pressure and the residue purified by Prep-HPLC with the following conditions: Column: welch Vltimate XB—C18, 50×250 mm, 10 μm; Mobile phase: 5-40% MeCN/0.1% aqueous NH₃; Detector: 220 nm; to afford 5-chloro-N-[2,4-difluoro-3-(2-[1-methylpyrazolo[4,3-b]pyridin-6-yl]ethyl)phenyl]-2-methoxypyridine-3-sulfonamide (2.3 mg, 13% yield) as a white solid.

LC-MS (ES, m/z): [M+H]⁺494
¹H NMR (300 MHz, Methanol-d₄) δ 8.31 (d, J=2.6 Hz, 1H), 8.24 (d, J=1.8 Hz, 1H), 8.13 (d, J=1.0 Hz, 1H), 7.97 (d, J=2.6 Hz, 1H), 7.77 (s, 1H), 7.39-7.20 (m, 1H), 6.93-6.80 (m, 1H), 4.03 (d, J=14.2 Hz, 6H), 3.05 (s, 4H).

### Example 30: Synthesis of 5-chloro-N-[2,4-difluoro-3-(2-[imidazo[1,5-al pyridin-7-yl]ethyl)phenyl]-2-methoxypyridine-3-sulfonamide (Compound 19)

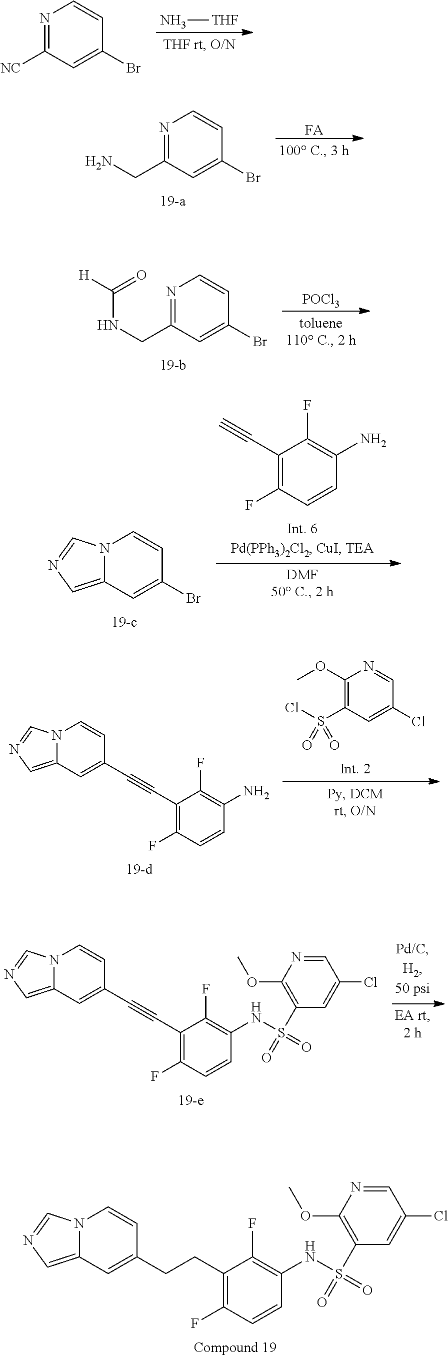

### Synthesis of 19-a: 1-(4-bromopyridin-2-yl)methanamine

BH₃·THF (82 mL, 1 M, 82 mmol, 5 equiv) was added dropwise to a stirred solution of 4-bromopyridine-2-carbonitrile (3.0 g, 16 mmol, 1 equiv) in THF (33 mL) at 0° C. under N₂ atmosphere and stirred at room temperature for 16 hours. The mixture was cooled to 0° C., and 2 M aqueous HCl (83 mL, 170 mmol, 10 eq) was added dropwise. The mixture was heated to reflux for 30 min and then cooled to room temperature. The mixture was basified to pH 8 with 2 M aqueous NaOH (100 mL).

The resulting mixture was extracted with THF (50 mL×3) and the combined organic layers washed with brine (50 mL), dried over anhydrous Na₂SO₄, filtered, and concentrated under reduced pressure to afford crude 1-(4-bromopyridin-2-yl)methanamine (3 g, crude) as a yellow oil which was used directly in next step without further purification.
LCMS (ES, m/z): [M+H]⁺: 187

### Synthesis of 19-b: N-[(4-bromopyridin-2-yl)methyl]formamide

1-(4-Bromopyridin-2-yl)methanamine (2.8 g, 15 mmol, 1 equiv) was dissolved in formic acid (8 mL) and stirred at 100° C. for 3 h. The mixture was concentrated under reduced pressure, saturated NaHCO₃ aqueous (45 mL) was added, and the resulting mixture extracted with DCM (3×45 mL). The combined organic extracts were washed with brine (30 mL), dried over anhydrous Na₂SO₄, filtered, and concentrated under reduced pressure. The crude residue was purified by silica gel column chromatography, eluting with PE:EA (2:1) to afford N-[(4-bromopyridin-2-yl)methyl]formamide (1.0 g, 30% yield for 2 steps) as a grey solid.

LCMS (ES, m/z): [M+H]⁺: 215

### Synthesis of 19-c: 7-bromoimidazo[1,5-a]pyridine

POCl₃ (784 mg, 5 mmol, 1.1 equiv) was added to a stirred solution of N-[(4-bromopyridin-2-yl)methyl]formamide (1.0 g, 4.6 mmol, 1 equiv) in toluene (10 mL) and the reaction mixture stirred at 110° C. for 2 h. The solution was concentrated under reduced pressure and the residue purified by silica gel chromatography, eluting with PE:EA (1:1) to afford 7-bromoimidazo[1,5-a]pyridine (750 mg, 82% yield) as a grey solid.

LCMS (ES, m/z): [M+H]⁺: 197

### Synthesis of 19-d: 2,6-difluoro-3-(2-[imidazo[1,5-a]pyridin-7-yl]ethynyl)aniline

TEA (340 mg, 3.3 mmol, 3 equiv), CuI (21 mg, 0.11 mmol, 0.1 equiv) and Pd(PPh₃)₂Cl₂ (78 mg, 0.11 mmol, 0.1 equiv) were added to a stirred solution of 7-bromoimidazo[1,5-a]pyridine (220 mg, 1.1 mmol, 1 equiv) and 3-ethynyl-2,6-difluoroaniline (200 mg, 1.3 mmol, 1.2 equiv) in DMF (5 mL) and the mixture purged with N₂ three times. The reaction mixture was then heated to 50° C. for 2 hours and then filtered. The filtrate was diluted with H₂O (10 mL) and extracted with EA (3×10 mL). The combined organic extracts were dried over anhydrous Na₂SO₄, filtered, and concentrated under reduced pressure. The residue was purified by silica gel chromatography, eluting with PE:EA (1:1) to afford 2,6-difluoro-3-(2-[imidazo[1,5-a]pyridin-7-yl]ethynyl)aniline (270 mg, 90% yield) as a brown solid.

LCMS (ES, m/z): [M+H]⁺: 270

### Synthesis of 19-e: 5-chloro-N-[2,4-difluoro-3-(2-[imidazo[1,5-a]pyridin-7-yl]ethynyl)phenyl]-2-methoxypyridine-3-sulfonamide

Pyridine (240 mg, 3 mmol, 3 equiv) and 5-chloro-2-methoxypyridine-3-sulfonyl chloride (290 mg, 1.2 mmol, 1.2 equiv) were added to a stirred solution of 2,4-difluoro-3-(2-[imidazo[1,5-a]pyridin-7-yl]ethynyl)aniline (270 mg, 1.0 mmol, 1 equiv) in DCM (5 mL) and the reaction mixture stirred at room temperature for 16 hours. The mixture was concentrated under reduced pressure and the residue purified by prep-HPLC with the following conditions: Column: welch Vltimate XB—C18, 50×250 mm, 10 μm; 20-60% MeCN/0.1% aqueous formic acid; Detector 220 nm; to afford 5-chloro-N-[2,4-difluoro-3-(2-[imidazo[1,5-a]pyridin-7-yl]ethynyl)phenyl]-2-methoxypyridine-3-sulfonamide (150 mg, 32% yield) as a brown solid.

LCMS (ES, m/z): [M+H]⁺: 475.
¹H NMR (300 MHz, DMSO-d₆) δ 10.48 (s, 1H), 8.53 (d, J=2.6 Hz, 1H), 8.49 (s, 1H), 8.38 (d, J=7.3 Hz, 1H), 8.09 (d, J=2.6 Hz, 1H), 7.94 (s, 1H), 7.53 (s, 1H), 7.40-7.33 (m, 1H), 7.23 (td, J=8.9, 1.5 Hz, 1H), 6.69 (dd, J=7.3, 1.6 Hz, 1H), 3.96 (s, 3H).

### Synthesis of Compound 19: 5-chloro-N-[2,4-difluoro-3-(2-[imidazo[1,5-a]pyridin-7-yl]ethyl)phenyl]-2-methoxypyridine-3-sulfonamide

10% Pd/C (10 mg) was added to a solution of 5-chloro-N-[2,4-difluoro-3-(2-[imidazo[1,5-a]pyridin-7-yl]ethynyl)phenyl]-2-methoxypyridine-3-sulfonamide (50 mg, 0.1 mmol, 1 equiv) in EA (5 mL) in a pressure tank. The mixture was stirred at room temperature for 2 h under H₂ (50 psi), then filtered through a Celite pad and concentrated under reduced pressure. The residue was purified by prep-HPLC with the following conditions Column: welch Vltimate XB—C18, 50×250 mm, 10 μm; Mobile Phase: 30-60% MeCN/aqueous formic acid; Detector 220 nm; to afford 5-chloro-N-[2,4-difluoro-3-(2-[imidazo[1,5-a]pyridin-7-yl]ethyl)phenyl]-2-methoxypyridine-3-sulfonamide (15 mg, 30% yield) as a white solid.

LCMS (ES, m/z): [M+H]⁺: 479.
¹H NMR (300 MHz, Methanol-d₄) δ 8.32 (d, J=2.6 Hz, 1H), 8.25 (s, 1H), 8.11 (d, J=7.2 Hz, 1H), 7.98 (d, J=2.6 Hz, 1H), 7.31-7.23 (m, 1H), 7.21 (s, 1H), 7.06 (s, 1H), 6.86 (td, J=9.0, 1.9 Hz, 1H), 6.46 (dd, J=7.3, 1.7 Hz, 1H), 4.03 (s, 3H), 2.95 (t, J=7.3 Hz, 2H), 2.75 (t, J=7.3 Hz, 2H).

### Example 31: Synthesis of N-[2,4-difluoro-3-[([4-methyl-1H-pyrazolo[3,4-b]pyridin-5-yl]oxy)methyl]phenyl]-5-fluoro-2-methoxypyridine-3-sulfonamide (Compound 20)

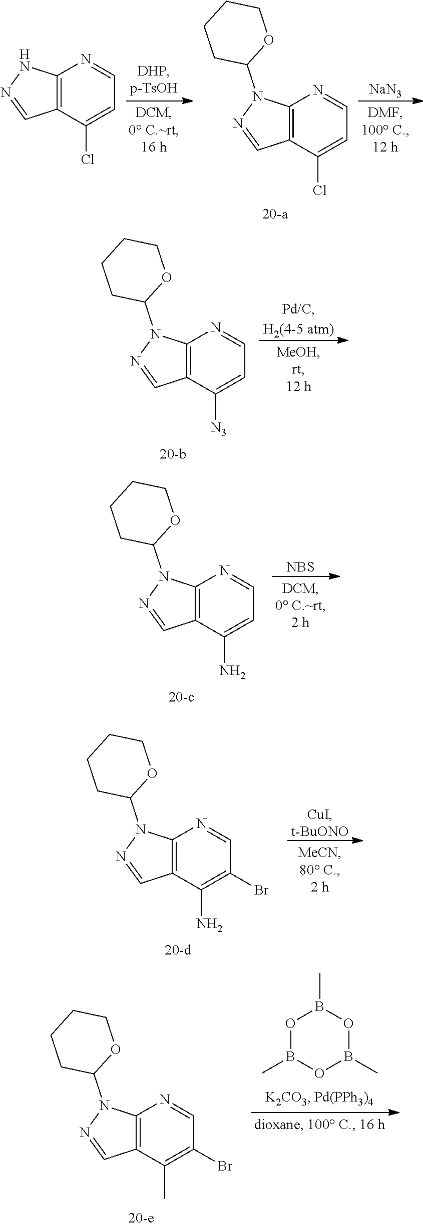

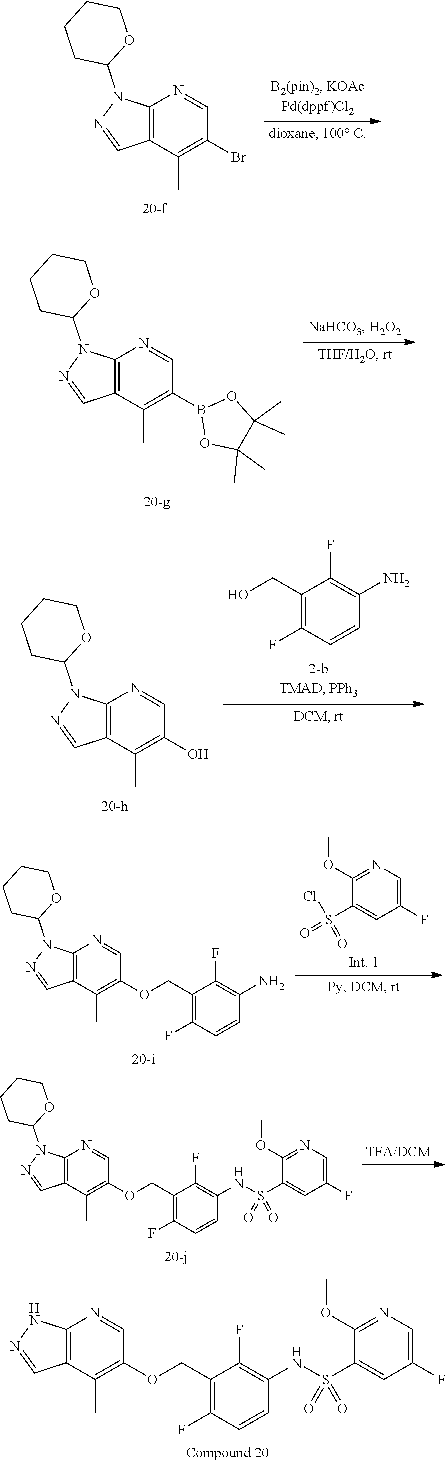

### Synthesis of 20-a: 4-chloro-1-(oxan-2-yl)pyrazolo[3,4-b]pyridine

Into a 1000 mL 3-necked round-bottom flask, was placed 4-chloro-1H-pyrazolo[3,4-b]pyridine (40 g, 260 mmol, 1 equiv), DCM (500 mL) and p-toluenesulfonic acid (9 g, 52 mmol, 0.2 equiv). This was followed by the addition of dihydropyran (65.7 g, 781 mmol, 3 equiv) dropwise with stirring at 0° C. The resulting solution was stirred for 16 h at room temperature, then concentrated under vacuum. The residue was applied to a silica gel column, eluting with ethyl acetate/petroleum ether (1:1) to afford 4-chloro-1-(oxan-2-yl)pyrazolo[3,4-b]pyridine (39 g, 57% yield) as a white solid.

LCMS (ES, m/z): [M+H]⁺: 238

### Synthesis of 20-b: 4-azido-1-(oxan-2-yl)pyrazolo[3,4-b]pyridine

Into a 500 mL 3-necked round-bottom flask, was placed 4-chloro-1-(oxan-2-yl)pyrazolo[3,4-b]pyridine (39 g, 164 mmol, 1 equiv), DMF (200 mL) and sodium azide (16 g, 0.25 mmol, 1.5 equiv). The resulting solution was stirred for 12 h at 100° C. in an oil bath. The resulting solution was diluted with 600 mL of H₂O and extracted with 2×300 mL of ethyl acetate. The combined organics were washed with 2×500 ml of brine and dried over anhydrous sodium sulfate, then concentrated under vacuum. The residue was applied to a silica gel column, eluting with ethyl acetate/petroleum ether (1:1) to afford 4-azido-1-(oxan-2-yl)pyrazolo[3,4-b]pyridine (24 g, 54% yield) as a white solid.

LCMS (ES, m/z): [M+H]⁺: 245

### Synthesis of 20-c: 1-(oxan-2-yl)pyrazolo[3,4-b]pyridin-4-amine

Into a 500 mL round-bottom flask, was placed 4-azido-1-(oxan-2-yl)pyrazolo[3,4-b]pyridine (24 g, 98 mmol, 1 equiv), methanol (200 mL) and Pd/C (2.4 g, 10%). This was hydrogenated at 4 atm hydrogen for 12 hrs, then filtered. The filtrate was concentrated under vacuum, and the residue applied to a silica gel column, eluting with ethyl acetate/petroleum ether (1:1) to afford 1-(oxan-2-yl)pyrazolo[3,4-b]pyridin-4-amine (13 g, 55% yield) as a white solid.

LCMS (ES, m/z): [M+H]⁺: 219

### Synthesis of 20-d: 5-bromo-1-(oxan-2-yl)pyrazolo[3,4-b]pyridin-4-amine

To a solution of 1-(oxan-2-yl)pyrazolo[3,4-b]pyridin-4-amine (5 g, 23 mmol, 1 equiv) in DCM (50 mL) was added NBS (4 g, 23 mmol, 1 equiv) in portions at 0° C. The resulting solution was stirred for 0.5 h at 0° C., then concentrated under reduced pressure. The residue was purified by column chromatography over silica gel (eluent: PE:EA=1:1) to afford 5-bromo-1-(oxan-2-yl)pyrazolo[3,4-b]pyridin-4-amine (6 g, 88% yield) as a yellow solid.

LCMS (ES, m/z): [M+H]⁺: 297

### Synthesis of 20-e: 5-bromo-4-iodo-1-(oxan-2-yl)pyrazolo[3,4-b]pyridine

A mixture of CuI (7.7 g, 40 mmol, 2 equiv) in MeCN (150 mL) was heated at 50° C. followed by addition of tBuONO (10.4 g, 101 mmol, 5 equiv). The reaction mixture was stirred at 50° C. for 0.5 h, when 5-bromo-1-(oxan-2-yl)pyrazolo[3,4-b]pyridin-4-amine (6 g, 20 mmol, 1 equiv) was added. The temperature was raised to 80° C. for 2 h, then cooled. The reaction was quenched with an aqueous saturated solution of NaHCO₃ (500 mL) and extracted with ethyl acetate (3×200 mL). The combined organics were washed with brine (2×100 mL), dried over anhydrous Na₂SO₄ and concentrated under reduced pressure. The residue was purified by column chromatography over silica gel (eluent: PE:EA=4:1) to afford 5-bromo-4-iodo-1-(oxan-2-yl)pyrazolo[3,4-b]pyridine (6 g, 73% yield) as a yellow solid.

LCMS (ES, m/z): [M+H]⁺: 408

### Synthesis of 20-f: 5-bromo-4-methyl-1-(oxan-2-yl)pyrazolo[3,4-b]pyridine

To a solution of 5-bromo-4-iodo-1-(oxan-2-yl)pyrazolo[3,4-b]pyridine (2 g, 4.9 mmol, 1 equiv) in dioxane (50 mL) were added a solution of trimethylboroxine in THE (1.23 g, 4.9 mmol, 1 equiv, 50%), K₂CO₃ (1.03 g, 14.7 mmol, 3 equiv) and Pd(PPh₃)₄ (566 mg, 0.49 mmol, 0.1 equiv) at room temperature under nitrogen atmosphere. The reaction mixture was stirred under nitrogen atmosphere at 100° C. for 16 h. The mixture was cooled to room temperature, water (200 mL) was added and this extracted with ethyl acetate (3×60 mL). The combined organics were washed with brine (2×50 mL), dried over anhydrous Na₂SO₄ and concentrated under reduced pressure. The crude product was purified by column chromatography over silica gel (eluent: PE:EA=3:1) to afford 5-bromo-4-methyl-1-(oxan-2-yl)pyrazolo[3,4-b]pyridine (1.2 g, 82% yield) as a yellow solid.

LCMS (ES, m/z): [M+H]⁺: 296

### Synthesis of 20-g: 4-methyl-1-(tetrahydro-2H-pyran-2-yl)-5-(4,4,5,5-tetramethyl-1,3,2-dioxaborolan-2-yl)-1H-pyrazolo[3,4-b]pyridine

To a solution of 5-bromo-4-methyl-1-(oxan-2-yl)pyrazolo[3,4-b]pyridine (1.2 g, 4.1 mmol, 1 equiv) in dioxane (50 mL) were added bis(pinacolato)diboron (2.06 g, 8.1 mmol, 2 equiv), KOAc (795 mg, 8.1 mmol, 2 equiv) and Pd(dppf)Cl₂ (297 mg, 0.41 mmol, 0.1 equiv) at room temperature under nitrogen atmosphere. The reaction mixture was heated at 100° C. for 8 h, then cooled. The mixture was diluted with water (100 mL) and extracted with ethyl acetate (3×100 mL). The combined organics were washed with brine (2×50 mL), dried over anhydrous Na2SO₄ and concentrated under reduced pressure to afford 4-methyl-1-(tetrahydro-2H-pyran-2-yl)-5-(4,4,5,5-tetramethyl-1,3,2-dioxaborolan-2-yl)-1H-pyrazolo[3,4-b]pyridine (2 g, crude) as a black solid which was used in the next step without further purification.

LCMS (ES, m/z): [M+H]⁺: 344

### Synthesis of 20-h: 4-methyl-1-(oxan-2-yl)pyrazolo[3,4-b]pyridin-5-ol

To a solution of 4-methyl-1-(oxan-2-yl)-5-(4,4,5,5-tetramethyl-1,3,2-dioxaborolan-2-yl)pyrazolo[3,4-b]pyridine (1.4 g, 4.1 mmol, 1 equiv) in THE (20 mL) and H₂O (20 mL) were added NaHCO₃ (860 mg, 10 mmol, 2.5 equiv) followed by addition of H₂O₂ (4.6 g, 41 mmol, 10 equiv, 30%) dropwise at room temperature. The resulting solution was stirred at room temperature for 1 h, then water (200 mL) was added. The mixture was extracted with ethyl acetate (3×60 mL), and the combined organics were washed with brine (2×50 mL), dried over anhydrous Na₂SO₄ and concentrated under reduced pressure. The residue was purified by column chromatography over silica gel (eluent: PE:EA=1:1) to afford 4-methyl-1-(oxan-2-yl)pyrazolo[3,4-b]pyridin-5-ol (600 mg, 63% yield) as a yellow solid.

LCMS (ES, m/z): [M+H]⁺: 234

### Synthesis of 20-i: 2,4-difluoro-3-([[4-methyl-1-(oxan-2-yl)pyrazolo[3,4-b]pyridin-5-yl]oxy]methyl)aniline

To a solution of 4-methyl-1-(oxan-2-yl)pyrazolo[3,4-b]pyridin-5-ol (400 mg, 1.72 mmol, 1 equiv) in DCM (10 mL) were added (3-amino-2,6-difluorophenyl)methanol (272 mg, 1.72 mmol, 1 equiv) and PPh₃ (674 mg, 2.6 mmol, 1.5 equiv) followed by addition of TMAD (442 mg, 2.57 mmol, 1.5 equiv) in portions at room temperature. The resulting solution was stirred at room temperature for 1 h. Water (200 mL) was added and the mixture extracted with ethyl acetate (3×80 mL). The combined organics were washed with brine (2×50 mL), dried over anhydrous Na₂SO₄ and concentrated under reduced pressure. The residue was purified by column chromatography over silica gel (eluent: PE:EA=1:1) to afford 2,4-difluoro-3-([[4-methyl-1-(oxan-2-yl)pyrazolo[3,4-b]pyridin-5-yl]oxy]methyl)aniline (300 mg, 46% yield) as a white solid.

LCMS (ES, m/z): [M+H]⁺: 375

### Synthesis of 20-j: N-[2,4-difluoro-3-([[4-methyl-1-(oxan-2-yl)pyrazolo[3,4-b]pyridin-5-yl]oxy]methyl)phenyl]-5-fluoro-2-methoxypyridine-3-sulfonamide

To a solution of 2,4-difluoro-3-([[4-methyl-1-(oxan-2-yl)pyrazolo[3,4-b]pyridin-5-yl]oxy]methyl)aniline (150 mg, 0.4 mmol, 1 equiv) in DCM (5 mL) was added pyridine (95 mg, 1.2 mmol, 3 equiv) and 5-fluoro-2-methoxypyridine-3-sulfonyl chloride (99 mg, 0.4 mmol, 1.1 equiv). The resulting solution was stirred at room temperature for 1 h, then concentrated under reduced pressure. The residue was purified by column chromatography over silica gel (eluent: PE:EA=2:1) to afford N-[2,4-difluoro-3-([[4-methyl-1-(oxan-2-yl)pyrazolo[3,4-b]pyridin-5-yl]oxy]methyl)phenyl]-5-fluoro-2-methoxypyridine-3-sulfonamide (80 mg, 35% yield) as a white solid.

LCMS (ES, m/z): [M+H]⁺: 564

### Synthesis of Compound 20: N-[2,4-difluoro-3-[([4-methyl-1H-pyrazolo[3,4-b]pyridin-5-yl]oxy)methyl]phenyl]-5-fluoro-2-methoxypyridine-3-sulfonamide

To a solution of N-[2,4-difluoro-3-([[4-methyl-1-(oxan-2-yl)pyrazolo[3,4-b]pyridin-5-yl]oxy]methyl)phenyl]-5-fluoro-2-methoxypyridine-3-sulfonamide (80 mg, 0.14 mmol, 1 equiv) in DCM (4 mL) was added TFA (1 mL) at room temperature. The resulting solution was stirred for 1 h, then concentrated under reduced pressure. The residue was purified by prep-HPLC with the following conditions: Column, welch Vltimate XB—C18, 50×250 mm, 10 μm; Mobile Phase: 25-60% MeCN/0.1% aqueous formic acid; Detector, 220 nm; to afford N-[2,4-difluoro-3-[([4-methyl-1H-pyrazolo[3,4-b]pyridin-5-yl]oxy)methyl]phenyl]-5-fluoro-2-methoxypyridine-3-sulfonamide (33 mg, 48% yield) as a white solid.

LCMS (ES, m/z): [M+H]⁺: 480
¹H NMR (300 MHz, DMSO-d₆) δ 13.47 (s, 1H), 10.36 (s, 1H), 8.45 (d, J=3.0 Hz, 1H), 8.34 (s, 1H), 8.12 (d, J=1.3 Hz, 1H), 7.94 (dd, J=7.3, 3.0 Hz, 1H), 7.35 (td, J=8.9, 6.0 Hz, 1H), 7.11 (td, J=9.0, 1.6 Hz, 1H), 5.13 (s, 2H), 3.91 (s, 3H), 2.25 (s, 3H).

### Example 32: Synthesis of N-[2,4-difluoro-3-([1H-pyrazolo[3,4-b]pyridin-5-yloxy]methyl)phenyl]-5-fluoro-2-methoxypyridine-3-sulfonamide (Compound 21)

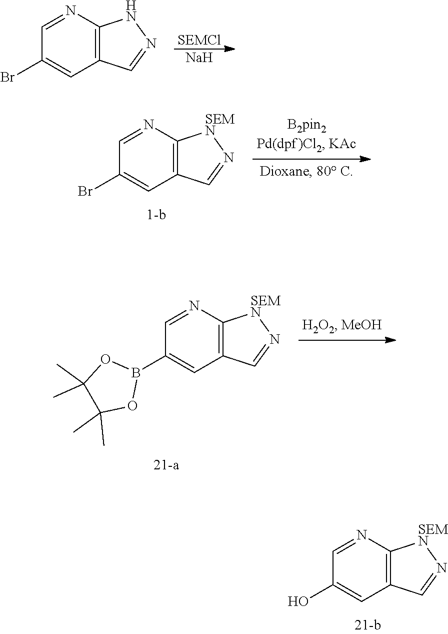

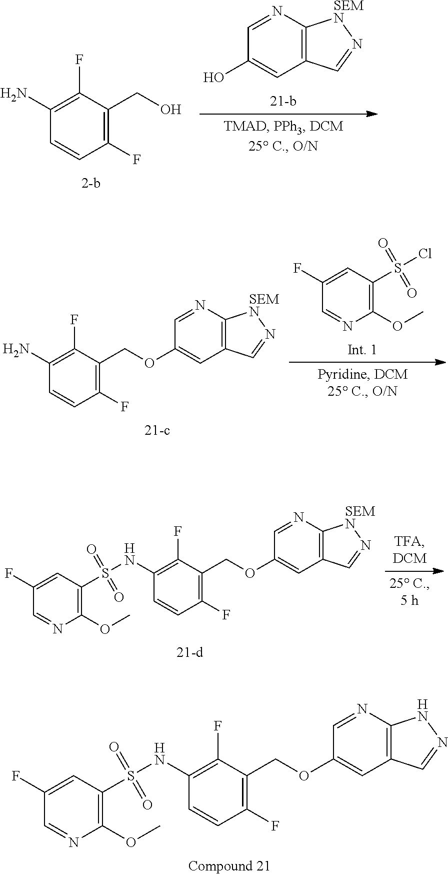 [SMILES: Nc1ccc(F)c(CO)c1F ; C[Si](C)(C)CCOCn1ncc2cc(O)cnc21 ; C[Si](C)(C)CCOCn1ncc2cc(OCc3c(F)ccc(N)c3F)cnc21 ; COc1ncc(F)cc1S(=O)(=O)Cl ; COc1ncc(F)cc1S(=O)(=O)Nc1ccc(F)c(COc2cnc3c(cnn3COCC[Si](C)(C)C)c2)c1F ; COc1ncc(F)cc1S(=O)(=O)Nc1ccc(F)c(COc2cnc3[nH]ncc3c2)c1F]

### Synthesis of 21-a: 5-(4,4,5,5-tetramethyl-1,3,2-dioxaborolan-2-yl)-1-[[2-(trimethylsilyl)ethoxy]methyl]pyrazolo[3,4-b]pyridine

Into a 500 mL vial was placed 5-bromo-1-[[2-(trimethylsilyl)ethoxy]methyl]pyrazolo[3,4-b]pyridine (8 g, 24 mmol, 1 equiv), dioxane (200 mL), bis(pinacolato)diboron (12.4 g, 49 mmol, 2 equiv), Pd(dppf)Cl₂ (1.78 g, 2 mmol, 0.1 equiv) and KOAc (4.8 g, 50 mmol, 2 equiv). The resulting solution was stirred for 2 h at 80° C. under N₂ atmosphere. The solids were removed by filtration and the filtrate was concentrated. The residue was applied to a silica gel column, eluting with ethyl acetate/petroleum ether (1:3) to give 5-(4,4,5,5-tetramethyl-1,3,2-dioxaborolan-2-yl)-1-[[2-(trimethylsilyl)ethoxy]methyl]pyrazolo[3,4-b]pyridine (11 g, crude) as an oil. The crude product was used directly.

LC-MS (ES, m/z): 376 [M+H]⁺

### Synthesis of 21-b: 1-[[2-(trimethylsilyl)ethoxy]methyl]pyrazolo[3,4-b]pyridin-5-ol

Into a 500 mL vial, was placed 5-(4,4,5,5-tetramethyl-1,3,2-dioxaborolan-2-yl)-1-[[2-(trimethylsilyl)ethoxy]methyl]pyrazolo[3,4-b]pyridine (12 g, 32 mmol, 1 equiv), MeOH (100 mL), Na₂SO₃ (252 mg, 2 mmol, 0.06 equiv), H₂O (100 mL) and H₂O₂ (35% in water) (32.8 g, 964 mmol, 30 equiv). The resulting solution was stirred overnight, then quenched by the addition of water (100 mL). The solids were collected by filtration and dried to give 1-[[2-(trimethylsilyl)ethoxy]methyl]pyrazolo[3,4-b]pyridin-5-ol (3.7 g, 44% yield) as a white solid.

LC-MS (ES, m/z): 266 [M+H]⁺

### Synthesis of 21-c: 2,4-difluoro-3-[[(1-[[2-(trimethylsilyl)ethoxy]methyl]pyrazolo[3,4-b]pyridin-5-yl)oxy]methyl]aniline

Into a 40 mL vial was placed 1-[[2-(trimethylsilyl)ethoxy]methyl]pyrazolo[3,4-b]pyridin-5-ol (133 mg, 0.5 mmol, 1 equiv), DCM (15 mL), (3-amino-2,6-difluorophenyl)methanol (88 mg, 0.6 mmol, 1.1 equiv), PPh₃ (197 mg, 0.8 mmol, 1.5 equiv) and TMAD (130 mg, 0.8 mmol, 1.5 equiv). The resulting solution was stirred overnight, then concentrated under vacuum. The crude product was purified by Flash-Prep-HPLC with the following conditions: Column, WelFlash TM C18-I, Spherical C18 20-40 μm, 120 g; mobile phase: 10-80% MeCN/0.1% aqueous formic acid; to afford 2,4-difluoro-3-[[(1-[[2-(trimethylsilyl)ethoxy]methyl]pyrazolo[3,4-b]pyridin-5-yl)oxy]methyl]aniline (130 mg, 64% yield) as a white solid.

LCMS (ES, m/z): 407 [M+H]⁺

### Synthesis of 21-d: N-(2,4-difluoro-3-[[(1-[[2-(trimethylsilyl)ethoxy]methyl]pyrazolo[3,4-b]pyridin-5-yl)oxy]methyl]phenyl)-5-fluoro-2-methoxypyridine-3-sulfonamide

Into a 40 mL vial was placed 2,4-difluoro-3-[[(1-[[2-(trimethylsilyl)ethoxy]methyl]pyrazolo[3,4-b]pyridin-5-yl)oxy]methyl]aniline (130 mg, 0.3 mmol, 1 equiv), DCM (8 mL), pyridine (79 mg, 1 mmol, 3 equiv) and 5-fluoro-2-methoxypyridine-3-sulfonyl chloride (108 mg, 0.5 mmol, 1.5 equiv). The resulting solution was stirred overnight, then concentrated under vacuum. The crude product was purified by Flash-Prep-HPLC with the following conditions: Column, WelFlash TM C18-I, Spherical C18 20-40 μm, 120 g; mobile phase: 5-85% MeCN/0.1% aqueous formic acid; Detector, 220 nm; to give N-(2,4-difluoro-3-[[(1-[[2-(trimethylsilyl)ethoxy]methyl]pyrazolo[3,4-b]pyridin-5-yl)oxy]methyl]phenyl)-5-fluoro-2-methoxypyridine-3-sulfonamide (110 mg, 58% yield) as a white solid.

LCMS (ES, m/z): 596 [M+H]⁺

### Synthesis of Compounds 21: N-[2,4-difluoro-3-([1H-pyrazolo[3,4-b]pyridin-5-yloxy]methyl)phenyl]-5-fluoro-2-methoxypyridine-3-sulfonamide

Into a 40 mL vial, was placed N-(2,4-difluoro-3-[[(1-[[2-(trimethylsilyl)ethoxy]methyl]pyrazolo[3,4-b]pyridin-5-yl)oxy]methyl]phenyl)-5-fluoro-2-methoxypyridine-3-sulfonamide (130 mg, 0.2 mmol, 1 equiv), DCM (5 mL) and TFA (1 mL). The resulting solution was stirred for 5 h, then concentrated under vacuum. The crude product was purified by Prep-HPLC with the following conditions: Column, welch Vltimate XB—C18, 50×250 mm, 10 μm; Mobile Phase: 20-60% MeCN/0.1% aqueous formic acid; to afford N-[2,4-difluoro-3-([1H-pyrazolo[3,4-b]pyridin-5-yloxy]methyl)phenyl]-5-fluoro-2-methoxypyridine-3-sulfonamide (53 mg, 52% yield) as a white solid.

LCMS (ES, m/z): [M+H]⁺: 466
¹H NMR (300 MHz, DMSO-d₆) δ 13.56 (s, 1H), 10.45 (s, 1H), 8.42 (d, J=3.0 Hz, 1H), 8.23 (d, J=2.7 Hz, 1H), 8.05 (s, 1H), 7.96 (dd, J=7.4, 3.0 Hz, 1H), 7.84 (d, J=2.8 Hz, 1H), 7.42-7.29 (m, 1H), 7.19-7.07 (m, 1H), 5.13 (s, 2H), 3.90 (s, 3H).

### Example 33: Synthesis of 5-chloro-N-[2,4-difluoro-3-[([4-methyl-1H-pyrazolo[3,4-b]pyridin-5-yl]oxy)methyl]phenyl]-2-methoxypyridine-3-sulfonamide (Compound 22)

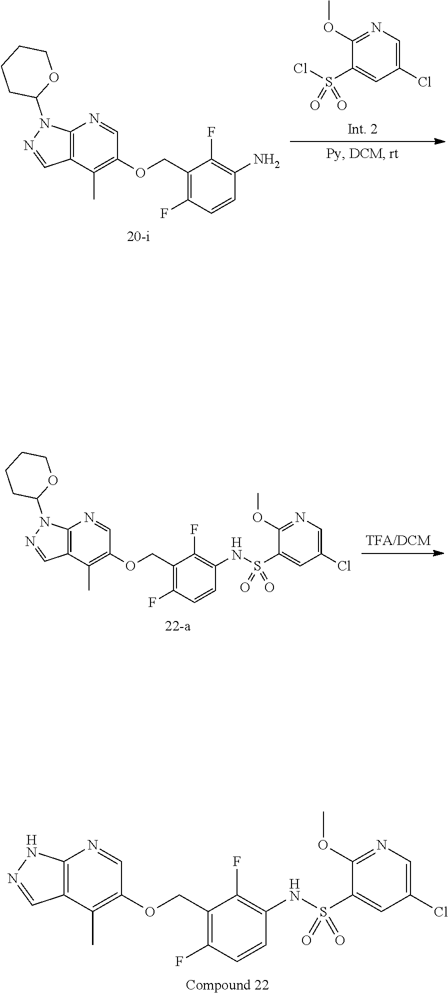 [SMILES: Cc1c(OCc2c(F)ccc(N)c2F)cnc2c1cnn2C1CCCCO1 ; COc1ncc(Cl)cc1S(=O)(=O)Cl ; COc1ncc(Cl)cc1S(=O)(=O)Nc1ccc(F)c(COc2cnc3c(cnn3C3CCCCO3)c2C)c1F ; COc1ncc(Cl)cc1S(=O)(=O)Nc1ccc(F)c(COc2cnc3[nH]ncc3c2C)c1F]

### Synthesis of 22-a: 5-chloro-N-[2,4-difluoro-3-([[4-methyl-1-(oxan-2-yl)pyrazolo[3,4-b]pyridin-5-yl]oxy]methyl)phenyl-2-methoxypyridine-3-sulfonamide

To a stirred solution of 2,4-difluoro-3-([[4-methyl-1-(oxan-2-yl)pyrazolo[3,4-b]pyridin-5-yl]oxy]methyl)aniline (150 mg, 0.4 mmol, 1 equiv) in DCM (5 mL) were added pyridine (95 mg, 1.2 mmol, 3 equiv) and 5-chloro-2-methoxypyridine-3-sulfonyl chloride (145 mg, 0.6 mmol, 1.5 equiv) at room temperature. The resulting solution was stirred for 1 h, then concentrated under reduced pressure. The residue was purified by column chromatography over silica gel (eluent: PE:EA=2:1) to afford 5-chloro-N-[2,4-difluoro-3-([[4-methyl-1-(oxan-2-yl)pyrazolo[3,4-b]pyridin-5-yl]oxy]methyl)phenyl]-2-methoxypyridine-3-sulfonamide (150 mg, 65% yield) as a white solid.

LCMS (ES, m/z): [M+H]⁺: 580

### Synthesis of Compound 22: 5-chloro-N-[2,4-difluoro-3-[([4-methyl-1H-pyrazolo[3,4-b]pyridin-5-yl]oxy)methyl]phenyl]-2-methoxypyridine-3-sulfonamide

To a stirred solution of 5-chloro-N-[2,4-difluoro-3-([[4-methyl-1-(oxan-2-yl)pyrazolo[3,4-b]pyridin-5-yl]oxy]methyl)phenyl]-2-methoxypyridine-3-sulfonamide (160 mg, 0.276 mmol, 1 equiv) in DCM (8 mL) was added TFA (2 mL) at room temperature. The resulting solution was stirred for 1 h, then concentrated under reduced pressure. The residue was purified by prep-HPLC with following conditions: Column, welch Vltimate XB—C18, 50×250 mm, 10 μm; Mobile Phase: 50-80% MeCN 0.1% aqueous formic acid; Detector, 220 nm; to afford 5-chloro-N-[2,4-difluoro-3-[([4-methyl-1H-pyrazolo[3,4-b]pyridin-5-yl]oxy)methyl]phenyl]-2-methoxypyridine-3-sulfonamide (80 mg, 58% yield) as a white solid.

LCMS (ES, m/z): [M+H]⁺: 496
¹H NMR (300 MHz, DMSO-d₆) δ 13.47 (s, 1H), 10.38 (s, 1H), 8.49 (d, J=2.6 Hz, 1H), 8.35 (s, 1H), 8.12 (s, 1H), 8.00 (d, J=2.6 Hz, 1H), 7.36 (td, J=8.9, 5.9 Hz, 1H), 7.12 (td, J=8.9, 1.6 Hz, 1H), 5.13 (s, 2H), 3.93 (s, 3H), 2.25 (s, 3H).

### Example 34: Synthesis of N-[2,4-difluoro-3-[([4-methoxy-1H-pyrazolo[3,4-b]pyridin-5-yl]oxy)methyl]phenyl]-5-fluoro-2-methoxypyridine-3-sulfonamide (Compound 23)

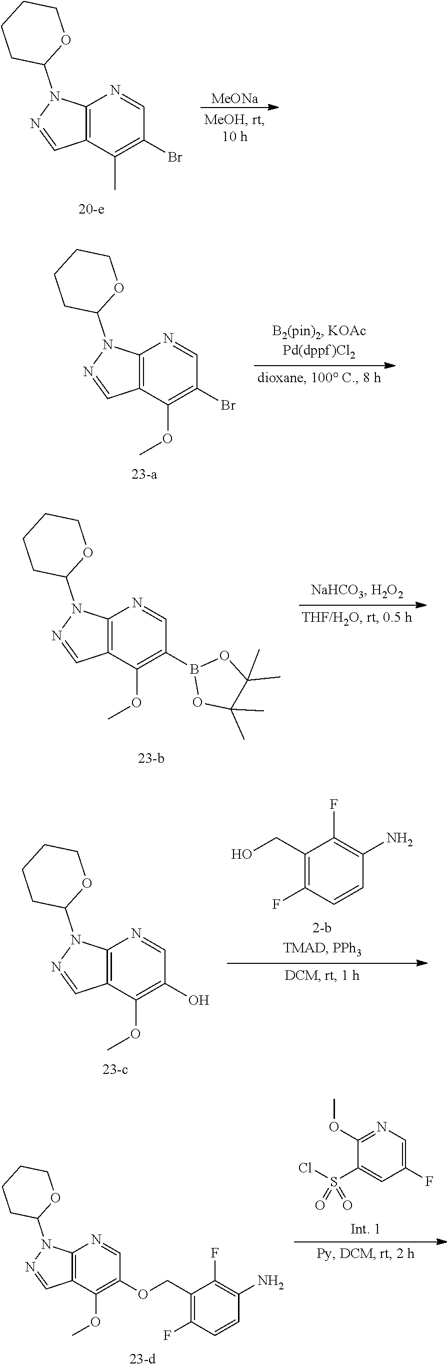

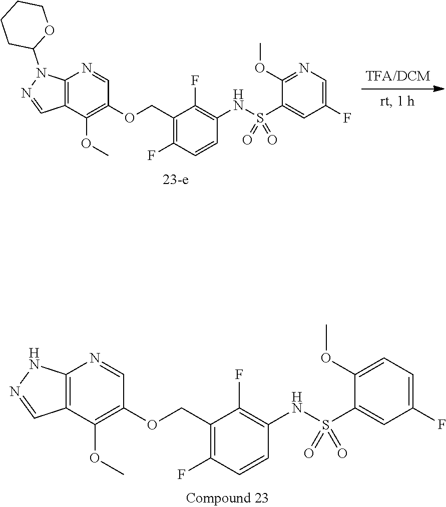 [SMILES: COc1ncc(F)cc1S(=O)(=O)Nc1ccc(F)c(COc2cnc3c(cnn3C3CCCCO3)c2OC)c1F ; COc1ccc(F)cc1S(=O)(=O)Nc1ccc(F)c(COc2cnc3[nH]ncc3c2OC)c1F]

### Synthesis of 23-a: 5-bromo-4-methoxy-1-(oxan-2-yl)pyrazolo[3,4-b]pyridine

To a solution of 5-bromo-4-iodo-1-(oxan-2-yl)pyrazolo[3,4-b]pyridine (1 g, 2.5 mmol, 1 equiv) in MeOH (20 mL) was added a solution of 30% NaOMe in MeOH (1.32 g, 7.35 mmol, 3 equiv) at room temperature. The resulting solution was stirred for 10 h then concentrated under reduced pressure. Water (100 mL) was added to the residue and this was extracted with ethyl acetate (3×60 mL). The combined organics were washed with brine (2×100 mL), dried over anhydrous Na₂SO₄, and concentrated under reduced pressure. The residue was purified by column chromatography over silica gel (eluent: PE:EA=4:1) to afford 5-bromo-4-methoxy-1-(oxan-2-yl)pyrazolo[3,4-b]pyridine (700 mg, 92% yield) as an off-white solid.

LCMS (ES, m/z): [M+H]⁺: 312

### Synthesis of 23-b: 4-methoxy-1-(oxan-2-yl)-5-(4,4,5,5-tetramethyl-1,3,2-dioxaborolan-2-yl)pyrazolo[3,4-b]pyridine

To a solution of 5-bromo-4-methoxy-1-(oxan-2-yl)pyrazolo[3,4-b]pyridine (600 mg, 1.9 mmol, 1 equiv) in dioxane (20 mL) were added bis(pinacolato)diboron (976 mg, 3.8 mmol, 2 equiv) and KOAc (377 mg, 3.8 mmol, 2 equiv) followed by addition of Pd(dppf)Cl₂ (140 mg, 0.2 mmol, 0.1 equiv) at room temperature under nitrogen atmosphere. The resulting solution was stirred at 100° C. for 8 h. After the reaction mixture was cooled and quenched with water (300 mL), it was extracted with ethyl acetate (3×100 mL). The combined organics were washed with brine (2×50 mL), dried over anhydrous Na₂SO₄, and concentrated under reduced pressure to afford 4-methoxy-1-(oxan-2-yl)-5-(4,4,5,5-tetramethyl-1,3,2-dioxaborolan-2-yl)pyrazolo[3,4-b]pyridine (600 mg) as a brown solid which was used in the next step directly without purification.

LCMS (ES, m/z): [M+H]⁺: 360

### Synthesis of 23-c: 4-methyl-1-(oxan-2-yl)pyrazolo[3,4-b]pyridin-5-ol

To a stirred solution of 4-methoxy-1-(oxan-2-yl)-5-(4,4,5,5-tetramethyl-1,3,2-dioxaborolan-2-yl)pyrazolo[3,4-b]pyridine (400 mg, 1.1 mmol, 1 equiv) in THE (5 mL) and water (5 mL) were added NaHCO₃ (233 mg, 2.7 mmol, 2.5 equiv) followed by addition of H₂O₂ (1.26 g, 11.1 mmol, 10 equiv, 30%) dropwise at room temperature. The resulting mixture was stirred for 0.5 h, then water (200 mL) was added. The mixture was extracted with ethyl acetate (3×60 mL). The combined organics were washed with brine (2×20 mL), dried over anhydrous Na₂SO₄, and concentrated under reduced pressure. The residue was purified by column chromatography over silica gel (eluent: PE:EA=1:1) to afford 4-methyl-1-(oxan-2-yl)pyrazolo[3,4-b]pyridin-5-ol (270 mg, 97% yield) as a yellow solid.

LCMS (ES, m/z): [M+H]⁺: 250

### Synthesis of 23-d: 2,4-difluoro-3-([[4-methoxy-1-(oxan-2-yl)pyrazolo[3,4-b]pyridin-5-yl]oxy]methyl)aniline

To a solution of 4-methoxy-1-(oxan-2-yl)pyrazolo[3,4-b]pyridin-5-ol (400 mg, 1.6 mmol, 1 equiv) in DCM (10 mL) were added (3-amino-2,6-difluorophenyl)methanol (255 mg, 1.6 mmol, 1 equiv) and PPh₃ (631 mg, 2.4 mmol, 1.5 equiv) followed by TMAD (414 mg, 2.4 mmol, 1.5 equiv) in portions at room temperature. The resulting solution was stirred for 1 h. Water (50 mL) was added and extracted with DCM (3×50 mL). The combined organics were washed with brine (2×20 mL), dried over anhydrous Na₂SO₄, and concentrated under reduced pressure. The residue was purified by column chromatography over silica gel (eluent: PE:EA=1:1) to afford 2,4-difluoro-3-([[4-methoxy-1-(oxan-2-yl)pyrazolo[3,4-b]pyridin-5-yl]oxy]methyl)aniline (300 mg, 47% yield) as a yellow solid.

LCMS (ES, m/z): [M+H]⁺: 391

### Synthesis of 23-e: N-[2,4-difluoro-3-([[4-methoxy-1-(oxan-2-yl)pyrazolo[3,4-b]pyridin-5-yl]oxy]methyl)phenyl]-5-fluoro-2-methoxypyridine-3-sulfonamide

To a stirred solution of 2,4-difluoro-3-([[4-methoxy-1-(oxan-2-yl)pyrazolo[3,4-b]pyridin-5-yl]oxy]methyl)aniline (150 mg, 0.3 mmol, 1 equiv) in DCM (5 mL) were added pyridine (91 mg, 1 mmol, 3 equiv) and 5-fluoro-2-methoxypyridine-3-sulfonyl chloride (130 mg, 0.45 mmol, 1.5 equiv) at room temperature. The resulting solution was stirred 2 h, then concentrated under reduced pressure. The residue was purified by column chromatography over silica gel (eluent: PE:EA=2:1) to afford N-[2,4-difluoro-3-([[4-methoxy-1-(oxan-2-yl)pyrazolo[3,4-b]pyridin-5-yl]oxy]methyl)phenyl]-5-fluoro-2-methoxypyridine-3-sulfonamide (180 mg, 81% yield) as a yellow solid.

LCMS (ES, m/z): [M+H]⁺: 580

### Synthesis of Compound 23: N-[2,4-difluoro-3-[([4-methoxy-1H-pyrazolo[3,4-b]pyridin-5-yl]oxy)methyl]phenyl]-5-fluoro-2-methoxypyridine-3-sulfonamide

To a stirred solution of N-[2,4-difluoro-3-([[4-methoxy-1-(oxan-2-yl)pyrazolo[3,4-b]pyridin-5-yl]oxy]methyl)phenyl]-5-fluoro-2-methoxypyridine-3-sulfonamide (100 mg, 0.17 mmol, 1 equiv) in DCM (8 mL) was added TFA (2 mL) at room temperature. The resulting solution was stirred for 1 h, then concentrated under reduced pressure. The residue was purified by prep-HPLC with the following conditions: Column, welch Vltimate XB—C18, 50×250 mm, 10 μm; Mobile Phase: 5-40% MeCN/0.1% aqueous ammonia; Detector: 220; nm to afford N-[2,4-difluoro-3-[([4-methoxy-1H-pyrazolo[3,4-b]pyridin-5-yl]oxy)methyl]phenyl]-5-fluoro-2-methoxypyridine-3-sulfonamide (40 mg, 29% yield) as a white solid.

LCMS (ES, m/z): [M+H]⁺: 496
¹H NMR (300 MHz, DMSO-d₆) δ 13.46 (s, 1H), 10.32 (s, 1H), 8.45 (d, J=3.0 Hz, 1H), 8.38 (d, J=1.4 Hz, 1H), 7.94 (q, J=3.3 Hz, 2H), 7.30 (td, J=8.8, 5.8 Hz, 1H), 7.14-6.94 (m, 1H), 5.04 (s, 2H), 4.25 (s, 3H), 3.92 (s, 3H).

### Example 35: Synthesis of 5-chloro-N-[2,4-difluoro-3-[([4-methoxy-1H-pyrazolo[3,4-b]pyridin-5-yl]oxy)methyl]phenyl]-2-methoxypyridine-3-sulfonamide (Compound 24)

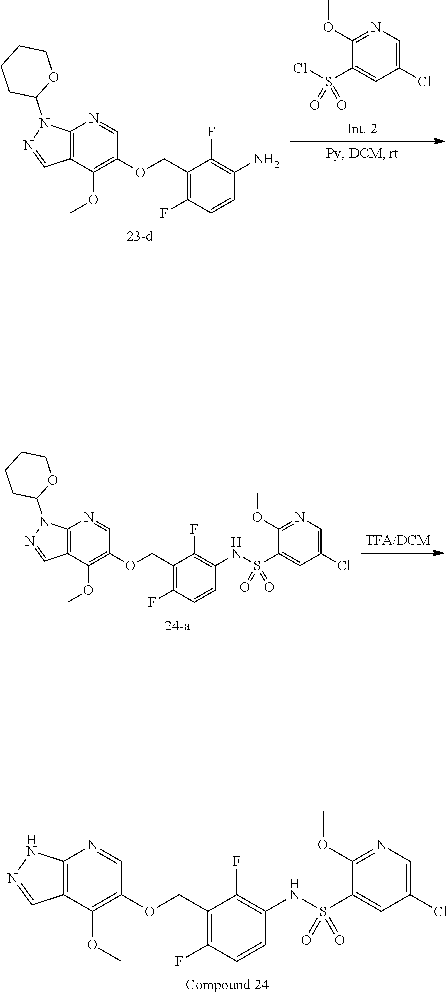 [SMILES: COc1c(OCc2c(F)ccc(N)c2F)cnc2c1cnn2C1CCCCO1 ; COc1ncc(Cl)cc1S(=O)(=O)Cl ; COc1ncc(Cl)cc1S(=O)(=O)Nc1ccc(F)c(COc2cnc3c(cnn3C3CCCCO3)c2OC)c1F ; COc1ncc(Cl)cc1S(=O)(=O)Nc1ccc(F)c(COc2cnc3[nH]ncc3c2OC)c1F]

### Synthesis of 24-a: 5-chloro-N-[2,4-difluoro-3-([[4-methoxy-1-(oxan-2-yl)pyrazolo[3,4-b]pyridin-5-yl]oxy]methyl)phenyl]-2-methoxypyridine-3-sulfonamide

To a stirred solution of 2,4-difluoro-3-([[4-methyl-1-(oxan-2-yl)pyrazolo[3,4-b]pyridin-5-yl]oxy]methyl)aniline (150 mg, 0.4 mmol, 1 equiv) in DCM (5 mL) was added pyridine (95 mg, 1.2 mmol, 3 equiv) and 5-chloro-2-methoxypyridine-3-sulfonyl chloride (139 mg, 0.5 mmol, 1.5 equiv) at room temperature. The resulting solution was stirred for 1 hour, then concentrated under reduced pressure. The residue was purified by column chromatography over silica gel (eluent: PE:EA=2:1) to afford 5-chloro-N-[2,4-difluoro-3-([[4-methoxy-1-(oxan-2-yl)pyrazolo[3,4-b]pyridin-5-yl]oxy]methyl)phenyl]-2-methoxypyridine-3-sulfonamide (170 mg, 74% yield) as a white solid.

LCMS (ES, m/z): [M+H]⁺: 596

### Synthesis of Compound 24: 5-chloro-N-[2,4-difluoro-3-[([4-methoxy-1H-pyrazolo[3,4-b]pyridin-5-yl]oxy)methyl]phenyl]-2-methoxypyridine-3-sulfonamide

To a stirred solution of 5-chloro-N-[2,4-difluoro-3-([[4-methoxy-1-(oxan-2-yl)pyrazolo[3,4-b]pyridin-5-yl]oxy]methyl)phenyl]-2-methoxypyridine-3-sulfonamide (100 mg, 168 μmol, 1 equiv) in DCM (8 mL) was added TFA (2 mL) at room temperature. The resulting solution was stirred for 1 hour then concentrated under reduced pressure. The residue was purified by prep-HPLC with the following conditions: Column, welch Vltimate XB—C18, 50×250 mm, 10 μm; Mobile Phase: 5-40% MeCN/0.1% aqueous ammonia; Detector, 220 nm; to afford 5-chloro-N-[2,4-difluoro-3-[([4-methoxy-1H-pyrazolo[3,4-b]pyridin-5-yl]oxy)methyl]phenyl]-2-methoxypyridine-3-sulfonamide (40 mg, 46% yield) as a white solid.

LCMS (ES, m/z): [M+H]⁺: 512
¹H NMR (300 MHz, DMSO-d₆) δ 13.46 (s, 1H), 10.34 (s, 1H), 8.49 (d, J=2.6 Hz, 1H), 8.38 (d, J=1.3 Hz, 1H), 8.01 (d, J=2.6 Hz, 1H), 7.96 (s, 1H), 7.38-7.22 (m, 1H), 7.07 (t, J=9.0 Hz, 1H), 5.04 (s, 2H), 4.25 (s, 3H), 3.92 (s, 3H).

### Example 36: Synthesis of 5-chloro-N-[2,4-difluoro-3-(1-[1H-pyrazolo[3,4-b]pyridin-5-yloxy]ethyl)phenyl]-2-methoxypyridine-3-sulfonamide (Compound 25)

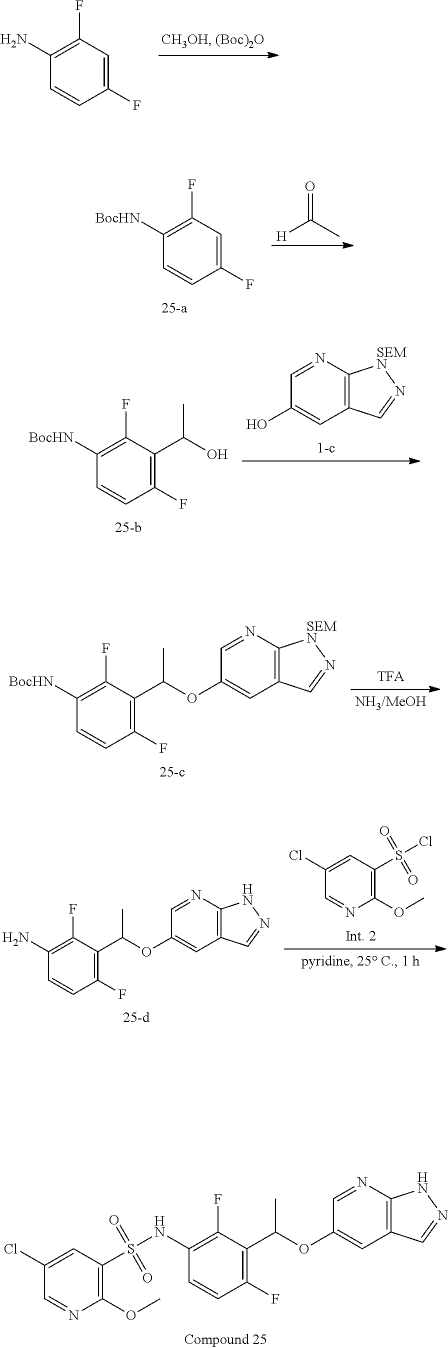

### Synthesis of 25-a: tert-butyl N-(2,4-difluorophenyl)carbamate

Into a 250 mL vial was placed 2,4-difluoroaniline (8 g, 62 mmol, 1 equiv), MeOH (150 mL) and di-tert-butyl dicarbonate (14.9 g, 74 mmol, 1.2 equiv). The resulting solution was stirred overnight at 40° C., then concentrated. The residue was applied to a silica gel column, eluting with ethyl acetate/petroleum ether (1:5) to give tert-butyl N-(2,4-difluorophenyl)carbamate (13 g, 92% yield) as a light yellow solid.

LC-MS (ES, m/z): 230 [M+H]⁺

### Synthesis of 25-b: tert-butyl N-[2,4-difluoro-3-(1-hydroxyethyl)phenyl]carbamate

Into a 250 mL vial was placed tert-butyl N-(2,4-difluorophenyl)carbamate (5 g, 22 mmol, 1 equiv) in THE (100 mL), and 2.5M n-BuLi in hexane (20 mL, 50 mmol, 2.3 equiv) was added dropwise at −78° C. The reaction was stirred for 1 h at −78° C., then acetaldehyde (880 mg, 22 mmol, 1 equiv) was dropwise at −78 degrees. The resulting solution was stirred for 1 h at −78° C. The reaction was quenched with water (50 mL) at low temperature, and the resulting solution extracted with ethyl acetate (50 mL×3). The combined organics were dried (Na₂SO₄) and concentrated. The residue was applied to a silica gel column, eluting with ethyl acetate/hexane (1:5) to give tert-butyl N-[2,4-difluoro-3-(1-hydroxyethyl)phenyl]carbamate (2.8 g, 47% yield) as a light yellow solid.

LC-MS (ES, m/z): 273 [M+H]⁺

### Synthesis of 25-c: tert-butyl-N-(2,4-difluoro-3-[1-[(1-[[2-(trimethylsilyl)ethoxy]methyl]pyrazolo[3,4-b]pyridin-5-yl)oxy]ethyl]phenyl)carbamate

Into a 40 mL vial was placed 1-[[2-(trimethylsilyl)ethoxy]methyl]pyrazolo[3,4-b]pyridin-5-ol (1.7 g, 6.4 mmol, 1 equiv), DCM (25 mL), tert-butyl N-[2,4-difluoro-3-(1-hydroxyethyl)phenyl]carbamate (1.9 g, 7.1 mmol, 1.1 equiv), TMAD (1.65 g, 10 mmol, 1.5 equiv) and PPh₃ (2.5 g, 10 mmol, 1.5 equiv). The resulting solution was stirred for 2 h then concentrated. The crude product was purified by Flash-Prep-HPLC with the following conditions: Column, WelFlash TM C18-I, Spherical C18 20-40 μm, 120 g; mobile phase: 10-80% MeCN/0.1% aqueous formic acid; Detector, 220 nm; to give tert-butyl N-(2,4-difluoro-3-[1-[(1-[[2-(trimethylsilyl)ethoxy]methyl]pyrazolo[3,4-b]pyridin-5-yl)oxy]ethyl]phenyl)carbamate (1.6 g, crude) as a light yellow oil.

LC-MS (ES, m/z): 521 [M+H]⁺

### Synthesis of 25-d: 2,4-difluoro-3-(1-[1H-pyrazolo[3,4-b]pyridin-5-yloxy]ethyl)aniline

Into a 100 mL 3-necked flask was placed tert-butyl N-(2,4-difluoro-3-[1-[(1-[[2-(trimethylsilyl)ethoxy]methyl]pyrazolo[3,4-b]pyridin-5-yl)oxy]ethyl]phenyl)carbamate (1.4 g, 2.7 mmol, 1 equiv), DCM (30 mL) and TFA (5 mL). The resulting solution was stirred for 20 min then concentrated. The crude product was purified by Flash-Prep-HPLC with the following conditions: Column, WelFlash TM C18-I, Spherical C18 20-40 μm, 120 g; mobile phase: 10-80% MeCN/0.1% aqueous formic acid; Detector, 220 nm; to give 2,4-difluoro-3-(1-[1H-pyrazolo[3,4-b]pyridin-5-yloxy]ethyl)aniline (310 mg, 40% yield) as a white solid.

LC-MS (ES, m/z): 291 [M+H]⁺

### Synthesis of Compound 25: Synthesis of 5-chloro-N-[2,4-difluoro-3-(1-[1H-pyrazolo[3,4-b]pyridin-5-yloxy]ethyl)phenyl]-2-methoxypyridine-3-sulfonamide

Into a 40 mL vial was placed 2,4-difluoro-3-(1-[1H-pyrazolo[3,4-b]pyridin-5-yloxy]ethyl)aniline (60 mg, 0.2 mmol, 1 equiv), pyridine (4 mL) and 5-chloro-2-methoxypyridine-3-sulfonyl chloride (100 mg, 0.4 mmol, 2 equiv). The resulting solution was stirred for 1 h then concentrated. The crude product was purified by Prep-HPLC with the following conditions: Column, welch Vltimate XB—C18, 50×250 mm, 10 μm; Mobile Phase: 25-65% MeCN/0.1% aqueous formic acid; Detector, 220 nm. This resulted in 5-chloro-N-[2,4-difluoro-3-(1-[1H-pyrazolo[3,4-b]pyridin-5-yloxy]ethyl)phenyl]-2-methoxypyridine-3-sulfonamide (32 mg, 31% yield) as a white solid.

LC-MS (ES, m/z): 496 [M+H]⁺
¹H NMR (300 MHz, DMSO-d₆) δ 13.51 (s, 1H), 10.32 (s, 1H), 8.34 (d, J=2.6 Hz, 1H), 8.19-8.11 (m, 1H), 7.98 (s, 1H), 7.89 (d, J=2.6 Hz, 1H), 7.55 (d, J=2.7 Hz, 1H), 7.29-7.14 (m, 1H), 7.01 (t, J=9.5 Hz, 1H), 5.80-5.68 (m, 1H), 3.84 (s, 3H), 1.66 (d, J=6.5 Hz, 3H).

### Example 37: Synthesis of 5-cyano-N-[2,4-difluoro-3-([1H-pyrazolo[3,4-b]pyridin-5-yloxy]methyl)phenyl]-2-methoxypyridine-3-sulfonamide (Compound 26)

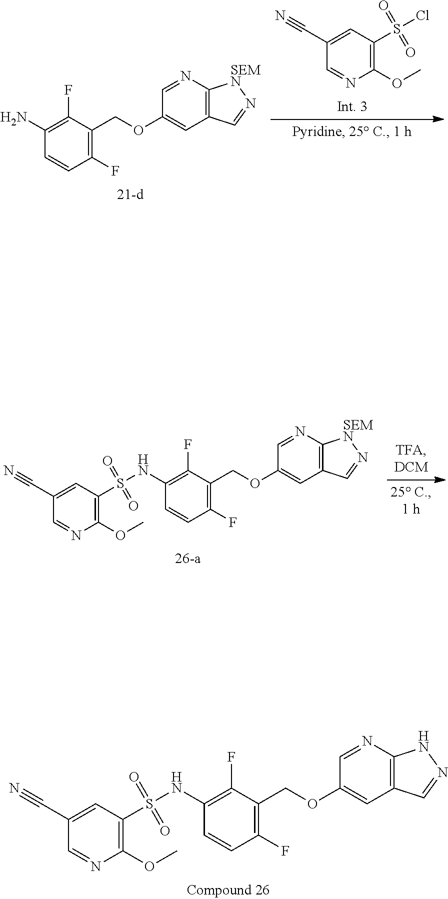 [SMILES: C[Si](C)(C)CCOCn1ncc2cc(OCc3c(F)ccc(N)c3F)cnc21 ; COc1ncc(C#N)cc1S(=O)(=O)Cl ; COc1ncc(C#N)cc1S(=O)(=O)Nc1ccc(F)c(COc2cnc3c(cnn3COCC[Si](C)(C)C)c2)c1F ; COc1ncc(C#N)cc1S(=O)(=O)Nc1ccc(F)c(COc2cnc3[nH]ncc3c2)c1F]

### Synthesis of 26-a: 5-cyano-N-(2,4-difluoro-3-[[(1-[[2-(trimethylsilyl)ethoxy]methyl]pyrazolo[3,4-b]pyridin-5-yl)oxy]methyl]phenyl)-2-methoxypyridine-3-sulfonamide

Into a 40 mL vial was placed 2,4-difluoro-3-[[(1-[[2-(trimethylsilyl)ethoxy]methyl]pyrazolo[3,4-b]pyridin-5-yl)oxy]methyl]aniline (150 mg, 0.37 mmol, 1 equiv), pyridine (4 mL) and 5-cyano-2-methoxypyridine-3-sulfonyl chloride (172 mg, 0.74 mmol, 2 equiv). The resulting solution was stirred for 1 h then concentrated. The crude product was purified by Flash-Prep-HPLC with the following conditions: Column, WelFlash TM C18-I, Spherical C18 20-40 μm, 120 g; mobile phase: 10-85% MeCN/0.1% aqueous formic acid; Detector, 220 nm; to give 5-cyano-N-(2,4-difluoro-3-[[(1-[[2-(trimethylsilyl)ethoxy]methyl]pyrazolo[3,4-b]pyridin-5-yl)oxy]methyl]phenyl)-2-methoxypyridine-3-sulfonamide (170 mg, 76% yield) as light yellow oil.

### Synthesis of Compound 26: 5-cyano-N-[2,4-difluoro-3-([1H-pyrazolo[3,4-b]pyridin-5-yloxy]methyl)phenyl]-2-methoxypyridine-3-sulfonamide

Into a 40 mL vial was placed 5-cyano-N-(2,4-difluoro-3-[[(1-[[(trimethylsilyl)methoxy]methyl]pyrazolo[3,4-b]pyridin-5-yl)oxy]methyl]phenyl)-2-methoxypyridine-3-sulfonamide (140 mg, 0.24 mmol, 1 equiv), DCM (5 mL) and TFA (1 mL). The resulting solution was stirred for 1 h then concentrated, and the pH of the residue was adjusted to 8 with ammonia (7 M in MeOH). The resulting solution was purified by Prep-HPLC with the following conditions: Column, welch Vltimate XB—C18, 50×250 mm, 10 μm; Mobile Phase: 5-30% MeCN/0.1% aqueous ammonia; Detector, 220 nm; to give 5-cyano-N-[2,4-difluoro-3-([1H-pyrazolo[3,4-b]pyridin-5-yloxy]methyl)phenyl]-2-methoxypyridine-3-sulfonamide (60 mg, 53% yield) as a white solid.

LC-MS (ES, m/z): [M+H]⁺=473 ¹H NMR (300 MHz, DMSO-d₆) δ 13.56 (s, 1H), 10.48 (s, 1H), 8.91 (d, J=2.2 Hz, 1H), 8.46 (d, J=2.2 Hz, 1H), 8.23 (d, J=2.7 Hz, 1H), 8.05 (s, 1H), 7.85 (d, J=2.7 Hz, 1H), 7.45-7.31 (m, 1H), 7.21-7.09 (m, 1H), 5.12 (s, 2H), 4.01 (s, 3H).

### Example 38: Synthesis of N-[3-[([4-cyano-1H-pyrazolo[3,4-b]pyridin-5-yl]oxy)methyl]-2,4-difluorophenyl]-5-fluoro-2-methoxypyridine-3-sulfonamide (Compound 27)

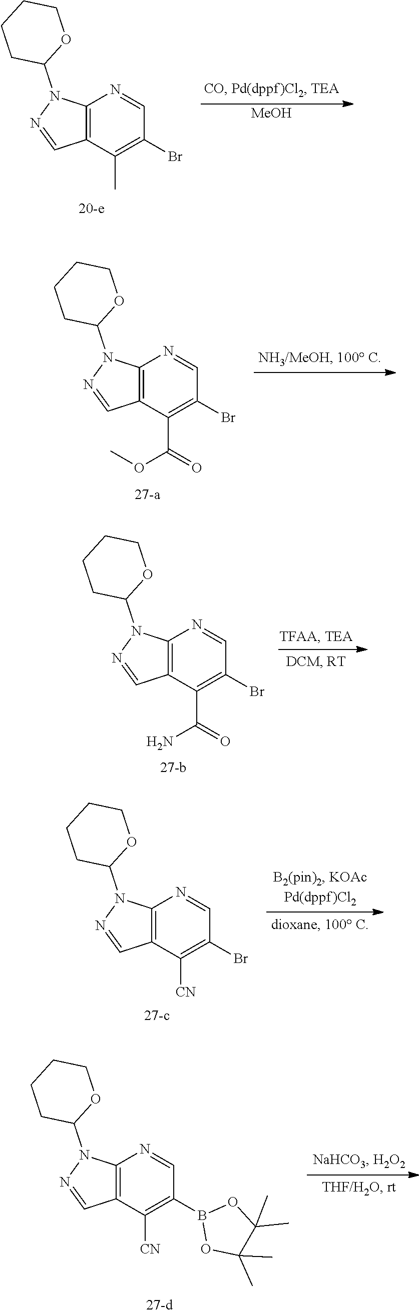 [SMILES: "MeOH" ; Cc1c(Br)cnc2c1cnn2C1CCCCO1 ; COC(=O)c1c(Br)cnc2c1cnn2C1CCCCO1 ; NC(=O)c1c(Br)cnc2c1cnn2C1CCCCO1 ; N#Cc1c(Br)cnc2c1cnn2C1CCCCO1 ; CC1(C)OB(c2cnc3c(cnn3C3CCCCO3)c2C#N)OC1(C)C]

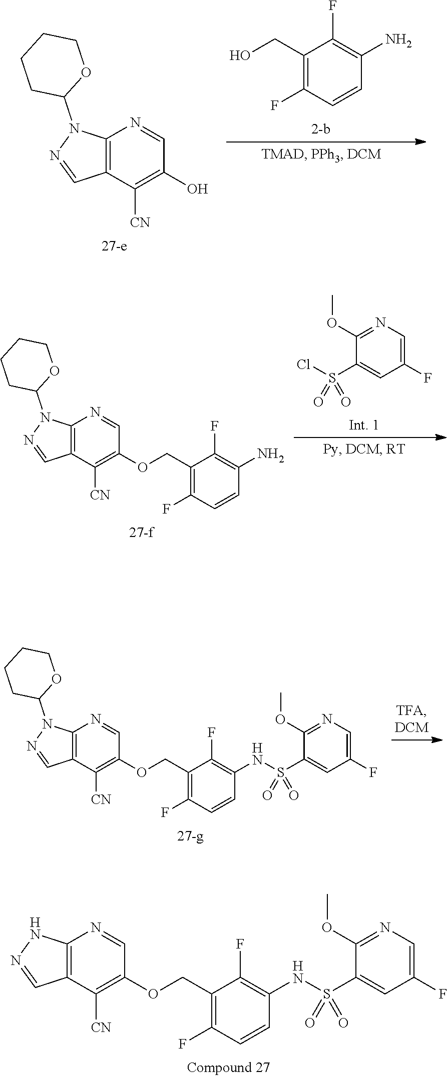 [SMILES: N#Cc1c(O)cnc2c1cnn2C1CCCCO1 ; Nc1ccc(F)c(CO)c1F ; N#Cc1c(OCc2c(F)ccc(N)c2F)cnc2c1cnn2C1CCCCO1 ; COc1ncc(F)cc1S(=O)(=O)Cl ; COc1ncc(F)cc1S(=O)(=O)Nc1ccc(F)c(COc2cnc3c(cnn3C3CCCCO3)c2C#N)c1F ; COc1ncc(F)cc1S(=O)(=O)Nc1ccc(F)c(COc2cnc3[nH]ncc3c2C#N)c1F]

### Synthesis of 27-a: methyl 5-bromo-1-(oxan-2-yl)pyrazolo[3,4-b]pyridine-4-carboxylate

To a solution of 5-bromo-4-iodo-1-(oxan-2-yl)pyrazolo[3,4-b]pyridine (1.5 g, 3.6 mmol, 1 equiv) in MeOH (20 mL) were added TEA (1.1 g, 11 mmol, 3 equiv) and Pd(dppf)Cl₂ (268 mg, 0.3 mmol, 0.1 equiv). The resulting solution was stirred at 60° C. under carbon monoxide atmosphere for 6 hrs. The mixture was filtered through celite and concentrated, and the residue was purified by column chromatography over silica gel (eluent: PE:EA=4:1) to afford methyl 5-bromo-1-(oxan-2-yl)pyrazolo[3,4-b]pyridine-4-carboxylate (1.2 g, 96% yield) as a brown solid.

LCMS (ES, m/z): [M+H]⁺: 340

### Synthesis of 27-b: 5-bromo-1-(oxan-2-yl)pyrazolo[3,4-b]pyridine-4-carboxamide

A solution of methyl 5-bromo-1-(oxan-2-yl)pyrazolo[3,4-b]pyridine-4-carboxylate (1.2 g, 3.5 mmol, 1 equiv) in 7 M ammonia solution in MeOH (20 ml) was stirred at 100° C. for 16 h. After cooling to room temperature, it was concentrated to give 5-bromo-1-(oxan-2-yl)pyrazolo[3,4-b]pyridine-4-carboxamide (800 mg, 70% yield) as a brown solid, which was used in next step directly without any purification.

LCMS (ES, m/z): [M+H]⁺: 325

### Synthesis of 27-c: 5-bromo-1-(oxan-2-yl)pyrazolo[3,4-b]pyridine-4-carbonitrile

To a solution of 5-bromo-1-(oxan-2-yl)pyrazolo[3,4-b]pyridine-4-carboxamide (800 mg, 2.4 mmol, 1 equiv) in THE (10 mL) were added TEA (622 mg, 6.1 mmol, 2.5 equiv) and TFA (1033 mg, 4.9 mmol, 2 equiv). The resulting solution was stirred for 2 hr, then concentrated. The residue was purified by column chromatography over silica gel (eluent: PE:EA=1:1) to afford 5-bromo-1-(oxan-2-yl)pyrazolo[3,4-b]pyridine-4-carbonitrile (700 mg, 93% yield) as a brown solid.

LCMS (ES, m/z): [M+H]⁺: 307

### Synthesis of 27-d: 1-(oxan-2-yl)-5-(4,4,5,5-tetramethyl-1,3,2-dioxaborolan-2-yl)pyrazolo[3,4-b]pyridine-4-carbonitrile

To a solution of 5-bromo-1-(oxan-2-yl)pyrazolo[3,4-b]pyridine-4-carbonitrile (800 mg, 2.6 mmol, 1 equiv) in dioxane (10 mL) were added bis(pinacolato)diboron (1.3 g, 5.2 mmol, 2 equiv), KOAc (511 mg, 5.2 mmol, 2 equiv) and Pd(dppf)Cl₂ (190 mg, 0.3 mmol, 0.1 equiv). The resulting solution was stirred at 100° C. under nitrogen for 4 h. After cooling, water (300 mL) was added and the mixture extracted with ethyl acetate (3×100 mL). The organic phase was washed with brine, dried over anhydrous Na₂SO₄, and concentrated to give the crude 1-(oxan-2-yl)-5-(4,4,5,5-tetramethyl-1,3,2-dioxaborolan-2-yl)pyrazolo[3,4-b]pyridine-4-carbonitrile (850 mg) as a brown solid which was used in next step directly without further purification.

LCMS (ES, m/z): [M+H]⁺: 355

### Synthesis of 27-e: 5-hydroxy-1-(oxan-2-yl)pyrazolo[3,4-b]pyridine-4-carbonitrile

To a solution of 1-(oxan-2-yl)-5-(4,4,5,5-tetramethyl-1,3,2-dioxaborolan-2-yl)pyrazolo[3,4-b]pyridine-4-carbonitrile (800 mg, 2.2 mmol, 1 equiv) in THE (5 mL) and H₂O (5 mL) were added NaHCO₃ (474 mg, 5.6 mmol, 2.5 equiv) and 30% H₂O₂ (2.5 g, 22.5 mmol, 10 equiv). The resulting solution was stirred for 4 hours, then diluted with water (200 mL). The mixture was extracted with ethyl acetate (3×100 mL), and the organics were washed with brine (3×50 mL), dried over anhydrous Na₂SO₄, before being concentrated. The residue was purified by column chromatography over silica gel (eluent: PE:EA=1:1) to give 5-hydroxy-1-(oxan-2-yl)pyrazolo[3,4-b]pyridine-4-carbonitrile (500 mg, 91% yield) as a white solid.

LCMS (ES, m/z): [M+H]⁺: 245

### Synthesis of 27-f: 5-[(3-amino-2,6-difluorophenyl)methoxy]-1-(oxan-2-yl)pyrazolo[3,4-b]pyridine-4-carbonitrile

To a solution of 5-hydroxy-1-(oxan-2-yl)pyrazolo[3,4-b]pyridine-4-carbonitrile (300 mg, 1.2 mmol, 1 equiv) in DCM (10 mL) were added (3-amino-2,6-difluorophenyl)methanol (234 mg, 1.4 mmol, 1.2 equiv), TMAD (317 mg, 1.8 mmol, 1.5 equiv) and PPh₃ (483 mg, 1.8 mmol, 1.5 equiv). The resulting solution was stirred at room temperature for 1 hour. The solution was concentrated, water (300 mL) was added and the mixture extracted with ethyl acetate (3×100 mL). The combined organics were washed with brine (3×50 mL), dried over anhydrous Na₂SO₄ and concentrated. The residue was purified by column chromatography over silica gel (eluent: PE:EA=2:1) to give 5-[(3-amino-2,6-difluorophenyl)methoxy]-1-(oxan-2-yl)pyrazolo[3,4-b]pyridine-4-carbonitrile (400 mg, 85% yield) as a white solid.

LCMS (ES, m/z): [M+H]⁺: 386

### Synthesis of 27-g: N-[3-([[4-cyano-1-(oxan-2-yl)pyrazolo[3,4-b]pyridin-5-yl]oxy]methyl)-2,4-difluorophenyl]-5-fluoro-2-methoxypyridine-3-sulfonamide

To a solution of 5-[(3-amino-2,6-difluorophenyl)methoxy]-1-(oxan-2-yl)pyrazolo[3,4-b]pyridine-4-carbonitrile (150 mg, 389 μmol, 1 equiv) in DCM (5 mL) were added 5-fluoro-2-methoxypyridine-3-sulfonyl chloride (131 mg, 584 μmol, 1.5 equiv) and pyridine (93 mg, 1.2 mmol, 3 equiv). The resulting solution was stirred for 1 hour at room temperature. The resulting solution was concentrated and purified by column chromatography over silica gel (eluent: PE:EA=4:1) to afford N-[3-([[4-cyano-1-(oxan-2-yl)pyrazolo[3,4-b]pyridin-5-yl]oxy]methyl)-2,4-difluorophenyl]-5-fluoro-2-methoxypyridine-3-sulfonamide (130 mg, 58% yield) as a white solid.

LCMS (ES, m/z): [M+H]⁺: 575

### Synthesis of Compound 27: N-[3-[([4-cyano-1H-pyrazolo[3,4-b]pyridin-5-yl]oxy)methyl]-2,4-difluorophenyl]-5-fluoro-2-methoxypyridine-3-sulfonamide

To a solution of N-[3-([[4-cyano-1-(oxan-2-yl)pyrazolo[3,4-b]pyridin-5-yl]oxy]methyl)-2,4-difluorophenyl]-5-fluoro-2-methoxypyridine-3-sulfonamide (100 mg, 174 μmol, 1 equiv) in DCM (8 mL) was added TFA (2 mL). The resulting solution was stirred at room temperature for 1 h. The mixture was concentrated, and the residue purified by prep-HPLC with following conditions: Column, welch Vltimate XB—C18, 50×250 mm, 10 μm; Mobile Phase: 20-50% MeCN/0.1% aqueous formic acid; Detector, 220 nm; to afford N-[3-[([4-cyano-1H-pyrazolo[3,4-b]pyridin-5-yl]oxy)methyl]-2,4-difluorophenyl]-5-fluoro-2-methoxypyridine-3-sulfonamide (40 mg, 46% yield) as a white solid.

LCMS (ES, m/z): [M+H]⁺: 491
¹H NMR (300 MHz, DMSO-d₆) δ 14.23 (s, 1H), 10.38 (s, 1H), 8.85 (s, 1H), 8.40 (d, J=3.0 Hz, 1H), 8.25 (s, 1H), 7.94 (dd, J=7.3, 3.0 Hz, 1H), 7.39 (td, J=9.0, 6.0 Hz, 1H), 7.17 (t, J=9.0 Hz, 1H), 5.47 (s, 2H), 3.87 (s, 3H).

### Example 39: Synthesis of 5-chloro-N-[3-[([4-cyano-1H-pyrazolo[3,4-b]pyridin-5-yl]oxy)methyl]-2,4-difluorophenyl]-2-methoxypyridine-3-sulfonamide (Compound 28)

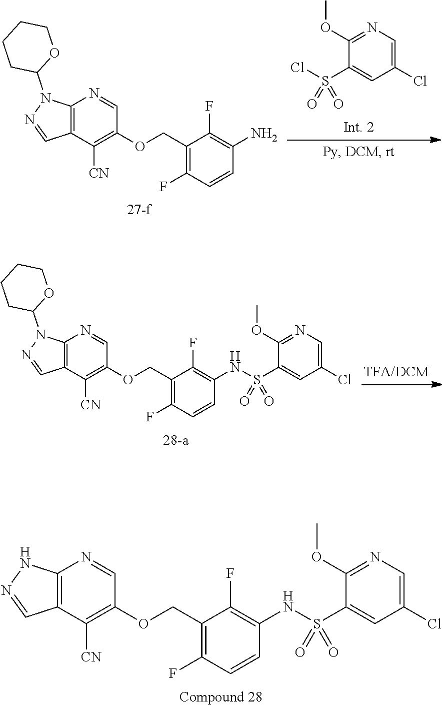 [SMILES: N#Cc1c(OCc2c(F)ccc(N)c2F)cnc2c1cnn2C1CCCCO1 ; COc1ncc(Cl)cc1S(=O)(=O)Cl ; COc1ncc(Cl)cc1S(=O)(=O)Nc1ccc(F)c(COc2cnc3c(cnn3C3CCCCO3)c2C#N)c1F ; COc1ncc(Cl)cc1S(=O)(=O)Nc1ccc(F)c(COc2cnc3[nH]ncc3c2C#N)c1F]

### Synthesis of 28-a: 5-chloro-N-[3-([[4-cyano-1-(oxan-2-yl)pyrazolo[3,4-b]pyridin-5-yl]oxy]methyl)-2,4-difluorophenyl]-2-methoxypyridine-3-sulfonamide

To a solution of 5-[(3-amino-2,6-difluorophenyl)methoxy]-1-(oxan-2-yl)pyrazolo[3,4-b]pyridine-4-carbonitrile (150 mg, 0.4 mmol, 1 equiv) in DCM (5 mL) were added 5-chloro-2-methoxypyridine-3-sulfonyl chloride (141 mg, 584 μmol, 1.5 equiv) and pyridine (93 mg, 1.2 mmol, 3 equiv). The resulting solution was stirred for 1 h at room temperature then concentrated under reduced pressure. The residue was purified by column chromatography over silica gel (eluent: PE:EA=5:1) to afford 5-chloro-N-[3-([[4-cyano-1-(oxan-2-yl)pyrazolo[3,4-b]pyridin-5-yl]oxy]methyl)-2,4-difluorophenyl]-2-methoxypyridine-3-sulfonamide (130 mg, 56% yield) as a white solid.

LCMS (ES, m/z): [M+H]⁺: 591

### Synthesis of Compound 28: 5-chloro-N-[3-[([4-cyano-1H-pyrazolo[3,4-b]pyridin-5-yl]oxy)methyl]-2,4-difluorophenyl]-2-methoxypyridine-3-sulfonamide

To a solution of 5-chloro-N-[3-([[4-cyano-1-(oxan-2-yl)pyrazolo[3,4-b]pyridin-5-yl]oxy]methyl)-2,4-difluorophenyl]-2-methoxypyridine-3-sulfonamide (120 mg, 0.2 mmol, 1 equiv) in DCM (8 mL) was added TFA (2 mL) at room temperature. The resulting solution was stirred for 1 h then concentrated under reduced pressure. The residue was purified by prep-HPLC with following conditions: Column, welch Vltimate XB—C18, 50×250 mm, 10 μm; Mobile Phase: 25-55% MeCN/0.1% aqueous formic acid; Detector, 220 nm; to afford 5-chloro-N-[3-[([4-cyano-1H-pyrazolo[3,4-b]pyridin-5-yl]oxy)methyl]-2,4-difluorophenyl]-2-methoxypyridine-3-sulfonamide (25 mg, 24% yield) as a white solid.

LCMS (ES, m/z): [M+H]⁺: 507
¹H NMR (300 MHz, DMSO-d₆) δ 14.23 (s, 1H), 10.40 (s, 1H), 8.85 (s, 1H), 8.43 (d, J=2.6 Hz, 1H), 8.25 (s, 1H), 8.00 (d, J=2.6 Hz, 1H), 7.51-7.32 (m, 1H), 7.17 (t, J=9.0 Hz, 1H), 5.47 (s, 2H), 3.88 (s, 3H).

### Example 40: Synthesis of 5-chloro-N-[2,4-difluoro-3-(1-[1H-pyrazolo[3,4-b]pyridin-5-ylamino]ethyl)phenyl]-2-methoxypyridine-3-sulfonamide (Compound 29)

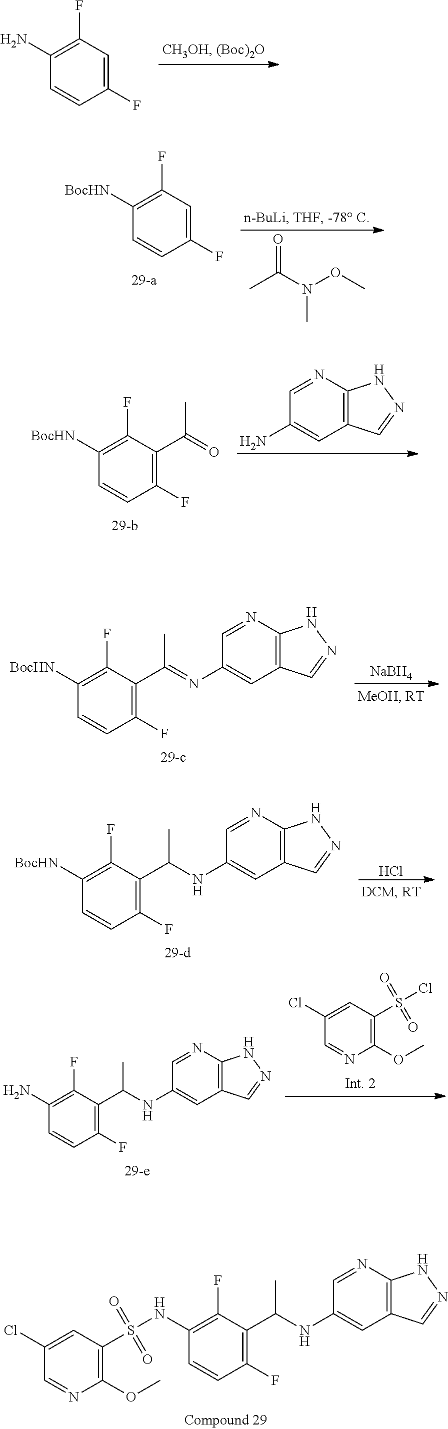

### Synthesis of 29-a: tert-butyl N-(2,4-difluorophenyl)carbamate

Into a 250 mL vial was placed 2,4-difluoroaniline (8 g, 62 mmol, 1 equiv), MeOH (150 mL) and tert-butyl 4,4-dimethyl-3-oxopentanoate (14.9 g, 74 mmol, 1.2 equiv). The resulting solution was stirred overnight at 40° C. The reaction mixture was concentrated and the residue was applied onto a silica gel column, eluting with ethyl acetate/petroleum ether (1:5) to give tert-butyl N-(2,4-difluorophenyl)carbamate (13 g, 92% yield) as a light yellow solid.

LC-MS (ES, m/z): [M+H]⁺=230

### Synthesis of 29-b: tert-butyl N-(3-acetyl-2,4-difluorophenyl)carbamate

Into a 50 mL 3-necked round-bottom flask, purged and maintained with an inert atmosphere of nitrogen, was placed tert-butyl N-(2,4-difluorophenyl)carbamate (1 g, 4.4 mmol, 1 equiv) and THE (20 mL). 2.5 M n-BuLi in hexane (3.9 mL, 9.6 mmol, 2.2 equiv) was added dropwise with stirring at −78° C. To this was added a solution of N-methoxy-N-methylacetamide (0.67 g, 6.5 mmol, 1.5 equiv) in THE (5 mL) dropwise with stirring at −78° C. The resulting solution was stirred for 5 h at 25° C. The reaction was then quenched by the addition of 50 mL of water. The resulting solution was extracted with 3×30 mL of ethyl acetate, and the combined organics washed with 50 ml of water and 50 mL of brine, then dried over anhydrous sodium sulfate and concentrated. The residue was purified by silica gel column chromatography, eluting with PE/EA (8:1) to afford tert-butyl N-(3-acetyl-2,4-difluorophenyl)carbamate (0.22 g, 19% yield) as colorless oil.

LCMS (ES, m/z): [M+H]⁺: 272

### Synthesis of 29-c: tert-butyl N-[2,4-difluoro-3-[(1E)-1-[1H-pyrazolo[3,4-b]pyridin-5-ylimino]ethyl]phenyl]carbamate

Into a 40 mL vial was placed tert-butyl N-(3-acetyl-2,4-difluorophenyl)carbamate (420 mg, 1.5 mmol, 1 equiv), 1H-pyrazolo[3,4-b]pyridin-5-amine (270 mg, 2 mmol, 1.3 equiv), DCE (15 mL) and Ti(Oi-Pr)₃Cl (1.2 g, 4.6 mmol, 3 equiv). The resulting solution was stirred for 48 h at 70° C., then cooled and diluted with 30 mL of MeOH. The resulting mixture was concentrated under vacuum. The residue was purified by silica gel column chromatography, eluting with PE:EA (2:1) to afford tert-butyl N-[2,4-difluoro-3-[(1E)-1-[1H-pyrazolo[3,4-b]pyridin-5-ylimino]ethyl]phenyl]carbamate (330 mg, 55% yield) as an off-white solid.

LCMS (ES, m/z): [M+H]⁺: 388

### Synthesis of 29-d: tert-butyl N-[2,4-difluoro-3-(1-[1H-pyrazolo[3,4-b]pyridin-5-ylamino]ethyl)phenyl]carbamate

Into a 100 mL 3-necked round-bottom flask was placed tert-butyl N-[2,4-difluoro-3-[(1E)-1-[1H-pyrazolo[3,4-b]pyridin-5-ylimino]ethyl]phenyl]carbamate (300 mg, 0.8 mmol, 1 equiv), THE (15 mL) and MeOH (15 mL). NaBH₄ (117 mg, 3.1 mmol, 4 equiv) was added in portions at room temperature. The resulting solution was stirred overnight, then quenched by the addition of 10 mL of MeOH. The resulting mixture was concentrated under vacuum. The residue was purified by silica gel column chromatography, eluting with PE:EA (1:1) to afford tert-butyl N-[2,4-difluoro-3-(1-[1H-pyrazolo[3,4-b]pyridin-5-ylamino]ethyl)phenyl]carbamate (100 mg, 33% yield) as an off-white solid.

LCMS (ES, m/z): [M+H]⁺: 390

### Synthesis of 29-e: N-[1-(3-amino-2,6-difluorophenyl)ethyl]-1H-pyrazolo[3,4-b]pyridin-5-amine

Into a 40 mL vial was placed tert-butyl N-[2,4-difluoro-3-(1-[1H-pyrazolo[3,4-b]pyridin-5-ylamino]ethyl)phenyl]carbamate (100 mg, 0.26 mmol, 1 equiv), DCM (5 mL) and 4M HCl in 1,4-dioxane (1 mL, 4 mmol). The resulting solution was stirred for 5 h then concentrated under vacuum. This resulted in N-[1-(3-amino-2,6-difluorophenyl)ethyl]-1H-pyrazolo[3,4-b]pyridin-5-amine hydrochloride (80 mg, crude) as a light brown solid.

LCMS (ES, m/z): [M+H]⁺: 290

### Synthesis of Compound 29: 5-chloro-N-[2,4-difluoro-3-(1-[1H-pyrazolo[3,4-b]pyridin-5-ylamino]ethyl)phenyl]-2-methoxypyridine-3-sulfonamide

Into a 40 mL vial was placed N-[1-(3-amino-2,6-difluorophenyl)ethyl]-1H-pyrazolo[3,4-b]pyridin-5-amine (70 mg, crude, 1 equiv), DCM (5 mL), Pyridine (79 mg, 1 mmol, 4 equiv) and 5-chloro-2-methoxypyridine-3-sulfonyl chloride (70 mg, 0.3 mmol, 1.2 equiv). The resulting solution was stirred overnight, then concentrated under vacuum. The crude product was purified by Prep-HPLC with the following conditions: Column, welch Vltimate XB—C18, 50×250 mm, 10 μm; Mobile Phase: 10-40% MeCN/0.1% aqueous formic acid; to afford 5-chloro-N-[2,4-difluoro-3-(1-[1H-pyrazolo[3,4-b]pyridin-5-ylamino]ethyl)phenyl]-2-methoxypyridine-3-sulfonamide (14 mg, 11% yield) as a white solid.

LCMS (ES, m/z): [M+H]⁺: 495
¹H NMR (300 MHz, DMSO-d₆) δ 13.13 (s, 1H), 10.20 (s, 1H), 8.32 (d, J=2.6 Hz, 1H), 8.08 (d, J=2.6 Hz, 1H), 7.87 (d, J=2.6 Hz, 1H), 7.77 (s, 1H), 7.20-6.94 (m, 2H), 6.81 (s, 1H), 6.10 (d, J=8.6 Hz, 1H), 4.85-4.74 (m, 1H), 3.86 (s, 3H), 1.52 (d, J=6.8 Hz, 3H).

### Example 41: Synthesis of N-(3-(((1H-pyrazolo[3,4-b]pyridin-5-yl)oxy)methyl)-2,4-difluorophenyl)-5-fluoro-2-methylpyridine-3-sulfonamide (Compound 30)

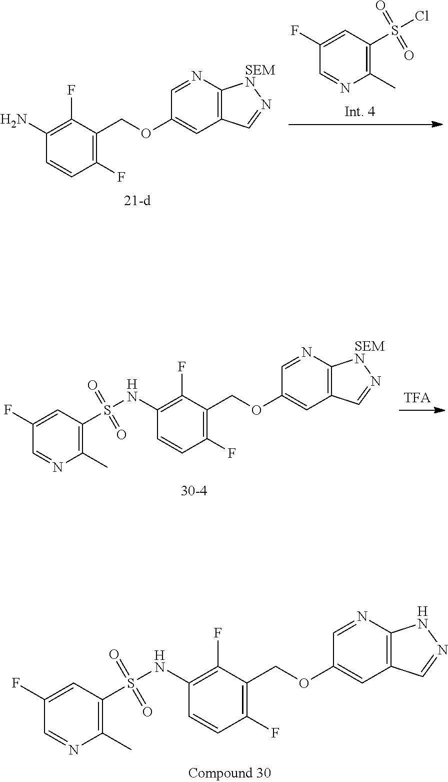 [SMILES: C[Si](C)(C)CCOCn1ncc2cc(OCc3c(F)ccc(N)c3F)cnc21 ; Cc1ncc(F)cc1S(=O)(=O)Cl ; Cc1ncc(F)cc1S(=O)(=O)Nc1ccc(F)c(COc2cnc3c(cnn3COCC[Si](C)(C)C)c2)c1F ; Cc1ncc(F)cc1S(=O)(=O)Nc1ccc(F)c(COc2cnc3[nH]ncc3c2)c1F]

### Synthesis of 30-a: N-(2,4-difluoro-3-(((1-((2-(trimethylsilyl)ethoxy)methyl)-1H-pyrazolo[3,4-b]pyridin-5-yl)oxy)methyl)phenyl)-5-fluoro-2-methylpyridine-3-sulfonamide

Into a 40 mL vial was placed 2,4-difluoro-3-[[(1-[[2-(trimethylsilyl)ethoxy]methyl]pyrazolo[3,4-b]pyridin-5-yl)oxy]methyl]aniline (150 mg, 0.37 mmol, 1 equiv), pyridine (4 mL) and 5-fluoro-2-methylpyridine-3-sulfonyl chloride (155 mg, 0.74 mmol, 2 equiv). The resulting solution was stirred for 1 h then concentrated. The crude product was purified by Flash-Prep-HPLC with the following conditions: Column, WelFlash TM C18-I, Spherical C18 20-40 μm, 120 g; mobile phase: 5-85% MeCN/0.1% aqueous formic acid; Detector, 220 nm; to give N-(2,4-difluoro-3-[[(1-[[2-(trimethylsilyl)ethoxy]methyl]pyrazolo[3,4-b]pyridin-5-yl)oxy]methyl]phenyl)-5-fluoro-2-methylpyridine-3-sulfonamide (170 mg, 80% yield) as light yellow oil.

LCMS (ES, m/z): [M+H]⁺: 580

### Synthesis of Compound 30: N-(3-(((1H-pyrazolo[3,4-b]pyridin-5-yl)oxy)methyl)-2,4-difluorophenyl)-5-fluoro-2-methylpyridine-3-sulfonamide

Into a 40 mL vial was placed 5-cyano-N-(2,4-difluoro-3-[[(1-[[(trimethylsilyl)methoxy]methyl]pyrazolo[3,4-b]pyridin-5-yl)oxy]methyl]phenyl)-2-methoxypyridine-3-sulfonamide (140 mg, 0.24 mmol, 1 equiv), DCM (5 mL) and TFA (1 mL). The resulting solution was stirred for 1 h then concentrated. The crude product was purified by Prep-HPLC with the following conditions: Column, welch Vltimate XB—C18, 50×250 mm, 10 μm; Mobile Phase: 15-70% MeCN/0.1% aqueous formic acid; Detector, 220 nm; to give N-(3-(((1H-pyrazolo[3,4-b]pyridin-5-yl)oxy)methyl)-2,4-difluorophenyl)-5-fluoro-2-methylpyridine-3-sulfonamide (60 mg, 54% yield) as a white solid.

LCMS (ES, m/z): [M+H]⁺: 450
¹H NMR (300 MHz, DMSO-d6) δ 13.55 (s, 1H), 10.71 (s, 1H), 8.70 (d, J=2.9 Hz, 1H), 8.21 (d, J=2.7 Hz, 1H), 8.05 (s, 1H), 7.87 (dd, J=8.3, 2.9 Hz, 1H), 7.82 (d, J=2.8 Hz, 1H), 7.42-7.28 (m, 1H), 7.16 (t, J=9.1 Hz, 1H), 5.11 (s, 2H), 2.75 (d, J=1.3 Hz, 3H).

### Example 42: Synthesis of 5-chloro-N-(2,4-difluoro-3-(2-(8-(methylamino)imidazo[1,2-a]pyrazin-3-yl)ethyl)phenyl)-2-methoxypyridine-3-sulfonamide (Compound 31)

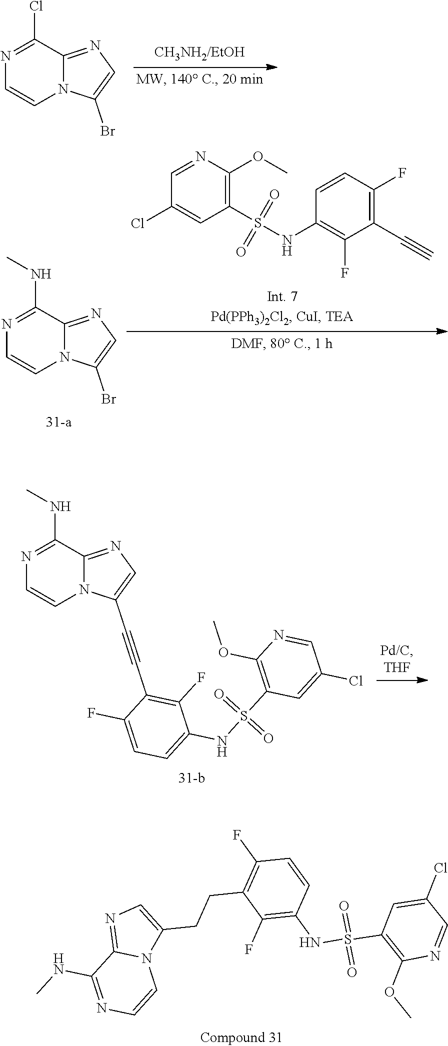

### Synthesis of 31-a: 3-bromo-N-methylimidazo[1,2-a]pyrazin-8-amine

A solution of 3-bromo-8-chloroimidazo[1,2-a]pyrazine (500 mg, 2.2 mmol, 1 equiv) in 33 wt. % CH₃NH₂/EtOH (5 mL) was heated by microwave at 150° C. for 0.5 h. After the mixture was cooled to room temperature, the mixture was filtered to afforded 3-bromo-N-methylimidazo[1,2-a]pyrazin-8-amine (450 mg, 92% yield) as a brown solid.

LCMS (ES, m/z): [M+H]⁺: 227

### Synthesis of 31-b: 5-chloro-N-(2,4-difluoro-3-[2-[8-(methylamino)imidazo[1,2-a]pyrazin-3-yl]ethynyl]phenyl)-2-methoxypyridine-3-sulfonamide

To a solution of 5-chloro-N-(3-ethynyl-2,4-difluorophenyl)-2-methoxypyridine-3-sulfonamide (500 mg, 1.4 mmol, 1 equiv) in DMF (10 mL) was added TEA (423 mg, 4.18 mmol, 3 equiv), 3-bromo-N-methylimidazo[1,2-a]pyrazin-8-amine (632 mg, 2.8 mmol, 2 equiv), CuI (26 mg, 139 μmol, 0.1 equiv) and Pd(PPh₃)₂Cl₂ (97 mg, 139 μmol, 0.1 equiv). The reaction mixture was stirred at 100° C. for 0.5 h, then cooled and quenched with water (200 mL). This was extracted with ethyl acetate (200 mL), and the organics washed with brine, dried over Na₂SO₄, and concentrated. The residue was purified by column chromatography over silica gel (eluent: PE:EA=1:1) to afford 5-chloro-N-(2,4-difluoro-3-[2-[8-(methylamino)imidazo[1,2-a]pyrazin-3-yl]ethynyl]phenyl)-2-methoxypyridine-3-sulfonamide (300 mg, 42% yield) as a yellow solid.

LCMS (ES, m/z): [M+H]⁺: 505
¹H NMR (300 MHz, DMSO-d₆) δ 10.53 (s, 1H), 8.50 (d, J=2.6 Hz, 1H), 8.08 (d, J=2.6 Hz, 1H), 8.00 (s, 1H), 7.76 (q, J=4.7 Hz, 1H), 7.58 (d, J=4.6 Hz, 1H), 7.53 (d, J=4.6 Hz, 1H), 7.39 (td, J=8.9, 5.9 Hz, 1H), 7.27-7.18 (m, 1H), 3.94 (s, 3H), 2.96 (d, J=4.7 Hz, 3H).

### Synthesis of Compound 31: 5-chloro-N-(2,4-difluoro-3-(2-(8-(methylamino)imidazo[1,2-a]pyrazin-3-yl)ethyl)phenyl)-2-methoxypyridine-3-sulfonamide

To a solution of 5-chloro-N-(2,4-difluoro-3-[2-[8-(methylamino)imidazo[1,2-a]pyrazin-3-yl]ethynyl]phenyl)-2-methoxypyridine-3-sulfonamide (100 mg, 0.2 mol, 1 equiv) in THE (10 mL) was added 10% Pd/C (43 mg). The resulting solution was stirred at room temperature under H₂ for 2 hours. The mixture was filtered through celite and concentrated, and the residue purified by HPLC with the following conditions: Column, welch Vltimate XB—C18, 50×250 mm, 10 μm; Mobile Phase: 30-60% MeCN/0.5% aqueous ammonia; to afford 5-chloro-N-(2,4-difluoro-3-(2-(8-(methylamino)imidazo[1,2-a]pyrazin-3-yl)ethyl)phenyl)-2-methoxypyridine-3-sulfonamide (20 mg, 20% yield) as a white solid.

LCMS (ES, m/z): [M+H]⁺: 509
¹H NMR (300 MHz, DMSO-d₆) δ 10.27 (s, 1H), 8.47 (s, 1H), 7.99 (d, J=2.6 Hz, 1H), 7.47 (d, J=4.7 Hz, 1H), 7.34 (d, J=4.9 Hz, 1H), 7.27 (d, J=4.7 Hz, 1H), 7.11 (d, J=6.9 Hz, 1H), 7.06 (s, 1H), 6.92 (t, J=9.0 Hz, 1H), 3.93 (s, 3H), 2.95 (m, 7H).

### Example 43: Synthesis of N-[3-(2-[8-aminoimidazo[1,2-a]pyrazin-3-yl]ethyl)-2,4-difluorophenyl]-5-chloro-2-methoxypyridine-3-sulfonamide (Compound 32)

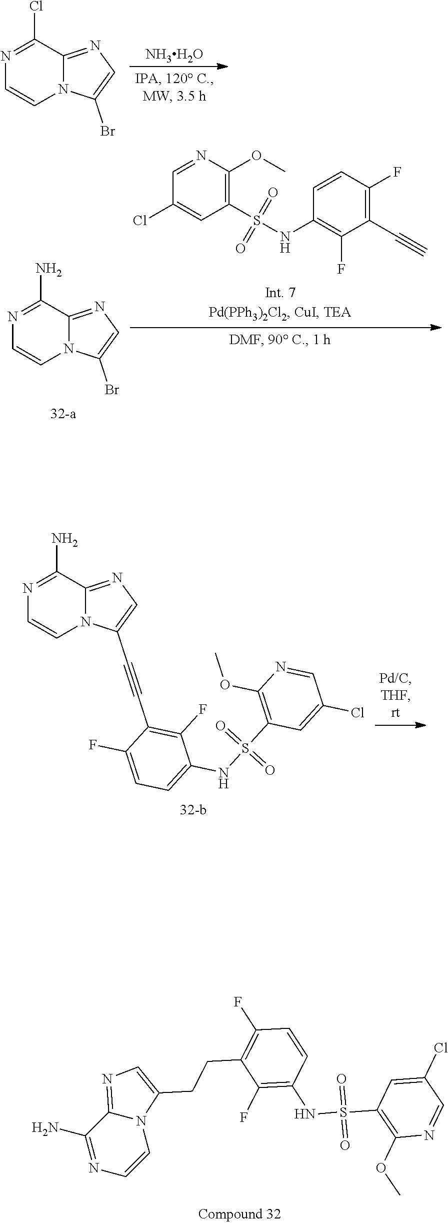

### Synthesis of 32-a: 3-bromoimidazo[1,2-a]pyrazin-8-amine

A solution of 3-bromo-8-chloroimidazo[1,2-a]pyrazine (1 g, 4.3 mmol, 1 equiv) and NH₃·H₂O (14 mL) in IPA (3 mL) was stirred for 3.5 h at 120° C. in a microwave reactor. The mixture was cooled, and the resulting solids were collected by filtration and washed with DCM (3×10 mL), then dried under vacuum to afford 3-bromoimidazo[1,2-a]pyrazin-8-amine (526 mg, 57% yield) as a white solid.

LCMS (ES, m/z): [M+H]⁺: 213

### Synthesis of 32-b: N-[3-(2-[8-aminoimidazo[1,2-a]pyrazin-3-yl]ethynyl)-2,4-difluorophenyl]-5-chloro-2-methoxypyridine-3-sulfonamide

To a stirred solution of 3-bromoimidazo[1,2-a]pyrazin-8-amine (100 mg, 0.5 mmol, 1 equiv), TEA (143 mg, 1.41 mmol, 3 equiv) and 5-chloro-N-(3-ethynyl-2,4-difluorophenyl)-2-methoxypyridine-3-sulfonamide (169 mg, 0.5 mmol, 1 equiv) in dimethylformamide (2 mL) were added Pd(PPh₃)₂Cl₂ (33 mg, 0.05 mmol, 0.1 equiv) and CuI (18 mg, 0.1 mmol, 0.2 equiv) in portions at room temperature under nitrogen atmosphere. The resulting mixture was stirred for 1 h at 90° C. then cooled. After filtration, the filtrate was concentrated under reduced pressure. The residue was purified by prep-HPLC with the following conditions: Column, welch Vltimate XB—C18, 50×250 mm, 10 μm; Mobile Phase: 10-55% MeCN/0.1% aqueous formic acid; Detector, 220 nm; to afford N-[3-(2-[8-aminoimidazo[1,2-a]pyrazin-3-yl]ethynyl)-2,4-difluorophenyl]-5-chloro-2-methoxypyridine-3-sulfonamide (33 mg, 14% yield) as a white solid.

LCMS (ES, m/z): [M+H]⁺: 491
¹H NMR (300 MHz, DMSO-d₆) δ 10.53 (s, 1H), 8.54 (d, J=2.6 Hz, 1H), 8.10 (d, J=2.7 Hz, 1H), 8.03 (s, 1H), 7.66-7.59 (m, 1H), 7.41 (td, J=8.9, 5.9 Hz, 1H), 7.26 (dd, J=17.1, 6.7 Hz, 3H), 3.96 (s, 3H), 2.48 (s, 1H).

### Synthesis of Compound 32: N-[3-(2-[8-aminoimidazo[1,2-a]pyrazin-3-yl]ethyl)-2,4-difluorophenyl]-5-chloro-2-methoxypyridine-3-sulfonamide

To a solution of N-[3-(2-[8-aminoimidazo[1,2-a]pyrazin-3-yl]ethynyl)-2,4-difluorophenyl]-5-chloro-2-methoxypyridine-3-sulfonamide (100 mg, 0.2 mol, 1 equiv) in THE (10 mL) was added 10% Pd/C (108 mg). The resulting solution was stirred at room temperature under a balloon of H₂ for 2 hours. The mixture was filtered through celite and the filtrate concentrated. The residue was purified by Flash-Prep-HPLC with the following conditions: Column, welch Vltimate XB—C18, 50×250 mm, 10 μm; Mobile Phase: 30-60% MeCN/0.5% aqueous NH₄HCO₃; Detector 254 nm; to afford N-[3-(2-[8-aminoimidazo[1,2-a]pyrazin-3-yl]ethyl)-2,4-difluorophenyl]-5-chloro-2-methoxypyridine-3-sulfonamide (50 mg, 50% yield) as a white solid.

LCMS (ES, m/z): [M+H]⁺: 495
¹H NMR (300 MHz, DMSO-d₆) δ 8.51 (d, J=2.6 Hz, 1H), 8.15 (s, 1H), 8.00 (d, J=2.6 Hz, 1H), 7.50 (d, J=4.7 Hz, 1H), 7.21 (d, J=4.7 Hz, 1H), 7.18-7.06 (m, 2H), 7.01-6.92 (m, 1H), 6.78 (s, 2H), 3.95 (s, 3H), 2.96 (q, J=4.9 Hz, 4H).

### Example 44: Synthesis of 5-chloro-N-[3-(2-[1-ethylpyrazolo[4,3-b]pyridin-6-yl]ethyl)-2,4-difluorophenyl]-2-methoxypyridine-3-sulfonamide (Compound 33)

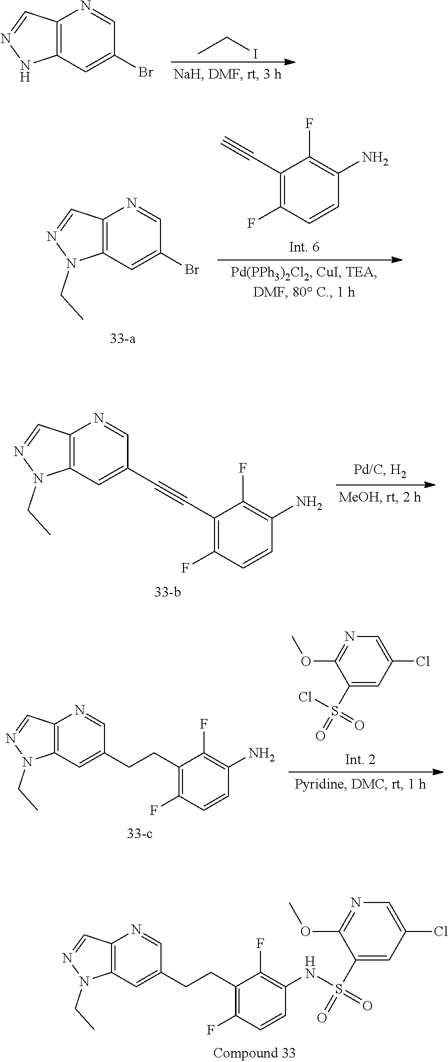

### Synthesis of 33-a: 6-bromo-1-ethylpyrazolo[4,3-b]pyridine

Into a 50 mL round-bottom flask was placed 6-bromo-1H-pyrazolo[4,3-b]pyridine (1 g, 5 mmol, 1 equiv), DMF (25 mL), NaH (404 mg, 10 mmol, 2 equiv, 60%) and ethyl iodide (945 mg, 6 mmol, 1.2 equiv). The resulting solution was stirred for 3 h at room temperature, then concentrated under vacuum. The residue was purified by silica gel column chromatography, eluting with PE:EA (3:1) to afford 6-bromo-1-ethylpyrazolo[4,3-b]pyridine (470 mg, 41% yield) as a white solid.

LCMS (ES, m/z): [M+H]⁺: 226

### Synthesis of 33-b: 3-(2-[1-ethylpyrazolo[4,3-b]pyridin-6-yl]ethynyl)-2,4-difluoroaniline

Into a 50 mL round-bottom flask was placed 6-bromo-1-ethylpyrazolo[4,3-b]pyridine (200 mg, 0.9 mmol, 1 equiv), DMF (10 mL), TEA (269 mg, 2.7 mmol, 3 equiv), CuI (17 mg, 0.09 mmol, 0.1 equiv), Pd(PPh₃)₂Cl₂ (62 mg, 0.09 mmol, 0.1 equiv) and 3-ethynyl-2,4-difluoroaniline (271 mg, 1.8 mmol, 2 equiv) in portions at room temperature under N₂ atmosphere. The reaction was stirred for 1 h at 80° C., then was cooled and diluted with 10 mL of H₂O. The resulting solution was extracted with 2×10 mL of ethyl acetate and the organics combined, washed with 3×10 mL of brine, dried over anhydrous sodium sulfate, then concentrated under vacuum. The residue was purified by silica gel column chromatography, eluting with PE:EA (3:1) to afford 3-(2-[1-ethylpyrazolo[4,3-b]pyridin-6-yl]ethynyl)-2,4-difluoroaniline (210 mg, 80% yield) as a dark yellow oil.

LCMS (ES, m/z): [M+H]⁺: 299

### Synthesis of 33-c: 3-(2-[1-ethylpyrazolo[4,3-b]pyridin-6-yl]ethyl)-2,4-difluoroaniline

Into a 30 mL sealed tube was placed 3-(2-[1-ethylpyrazolo[4,3-b]pyridin-6-yl]ethynyl)-2,4-difluoroaniline (210 mg, 0.7 mmol, 1 equiv), MeOH (4 mL), Pd/C (100 mg) and H₂ (20 atm.). The reaction was stirred for 2 h at room temperature. The mixture was filtered, and the filtrate concentrated under vacuum to give crude 3-(2-[1-ethylpyrazolo[4,3-b]pyridin-6-yl]ethyl)-2,4-difluoroaniline (202 mg) as a colorless oil which was used in next step directly without further purification.

LCMS (ES, m/z): [M+H]⁺: 303

### Synthesis of Compound 33: 5-chloro-N-[3-(2-[1-ethylpyrazolo[4,3-b]pyridin-6-yl]ethyl)-2,4-difluorophenyl]-2-methoxypyridine-3-sulfonamide

Into a 50 mL round-bottom flask was placed 3-(2-[1-ethylpyrazolo[4,3-b]pyridin-6-yl]ethyl)-2,4-difluoroaniline (100 mg, 0.3 mmol, 1 equiv), pyridine (5 mL), 5-chloro-2-methoxypyridine-3-sulfonyl chloride (80 mg, 0.3 mmol, 1 equiv) and DCM (0.1 mL). The reaction was stirred for 2 h at room temperature, then concentrated under vacuum. The residue was purified by prep-HPLC with the following conditions: Column, welch Vltimate XB—C18, 50×250 mm, 10 μm; Mobile Phase: 25-60% MeCN/0.1% aqueous formic acid; to afford 5-chloro-N-[3-(2-[1-ethylpyrazolo[4,3-b]pyridin-6-yl]ethyl)-2,4-difluorophenyl]-2-methoxypyridine-3-sulfonamide (30 mg, 18% yield) as a white solid.

LCMS (ES, m/z): [M+H]⁺: 508
¹H NMR (300 MHz, DMSO-d₆) δ 10.24 (s, 1H), 8.50 (d, J=2.6 Hz, 1H), 8.22 (dd, J=10.9, 1.4 Hz, 2H), 7.99 (d, J=2.6 Hz, 1H), 7.91-7.84 (m, 1H), 7.13 (td, J=8.9, 5.9 Hz, 1H), 6.98 (td, J=9.0, 1.6 Hz, 1H), 4.41 (q, J=7.2 Hz, 2H), 3.94 (s, 3H), 2.97-2.87 (m, 4H), 1.37 (t, J=7.2 Hz, 3H).

### Example 45: Synthesis of N-[3-(2-[1-ethylpyrazolo[4,3-b]pyridin-6-yl]ethyl)-2,4-difluorophenyl]-5-fluoro-2-methoxypyridine-3-sulfonamide (Compound 34)

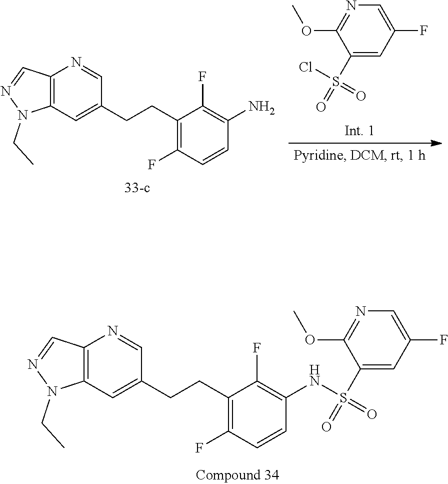 [SMILES: CCn1ncc2ncc(CCc3c(F)ccc(N)c3F)cc21 ; COc1ncc(F)cc1S(=O)(=O)Cl ; CCn1ncc2ncc(CCc3c(F)ccc(NS(=O)(=O)c4cc(F)cnc4OC)c3F)cc21]

### Synthesis of Compound 34: N-[3-(2-[1-ethylpyrazolo[4,3-b]pyridin-6-yl]ethyl)-2,4-difluorophenyl]-5-fluoro-2-methoxypyridine-3-sulfonamide

Into an 8 mL vial was placed 3-(2-[1-ethylpyrazolo[4,3-b]pyridin-6-yl]ethyl)-2,4-difluoroaniline (100 mg, 0.3 mmol, 1 equiv), pyridine (2 mL) and 5-fluoro-2-methoxypyridine-3-sulfonyl chloride (112 mg, 0.5 mmol, 1.5 equiv) in DCM (0.1 mL). The resulting solution was stirred for 1 h at room temperature, then concentrated under vacuum. The residue was purified by prep-HPLC with the following conditions: Column, welch Vltimate XB—C18, 50×250 mm, 10 μm; Mobile Phase: 20-50% MeCN/0.1% aqueous formic acid; to afford N-[3-(2-[1-ethylpyrazolo[4,3-b]pyridin-6-yl]ethyl)-2,4-difluorophenyl]-5-fluoro-2-methoxypyridine-3-sulfonamide (30 mg, 18% yield) as a white solid.

LCMS (ES, m/z): [M+H]⁺: 492
¹H NMR (300 MHz, DMSO-d₆) δ 10.22 (s, 1H), 8.46 (d, J=3.0 Hz, 1H), 8.21 (dd, J=8.9, 1.4 Hz, 2H), 7.97-7.84 (m, 2H), 7.12 (td, J=8.8, 5.9 Hz, 1H), 6.97 (td, J=9.0, 1.6 Hz, 1H), 4.40 (q, J=7.2 Hz, 2H), 3.93 (s, 3H), 2.94 (d, J=10.8 Hz, 4H), 1.36 (t, J=7.2 Hz, 3H).

### Example 46: Synthesis of 5-chloro-N-[2,4-difluoro-3-(2-[1-isopropylpyrazolo[4,3-b]pyridin-6-yl]ethyl)phenyl]-2-methoxypyridine-3-sulfonamide (Compound 35)

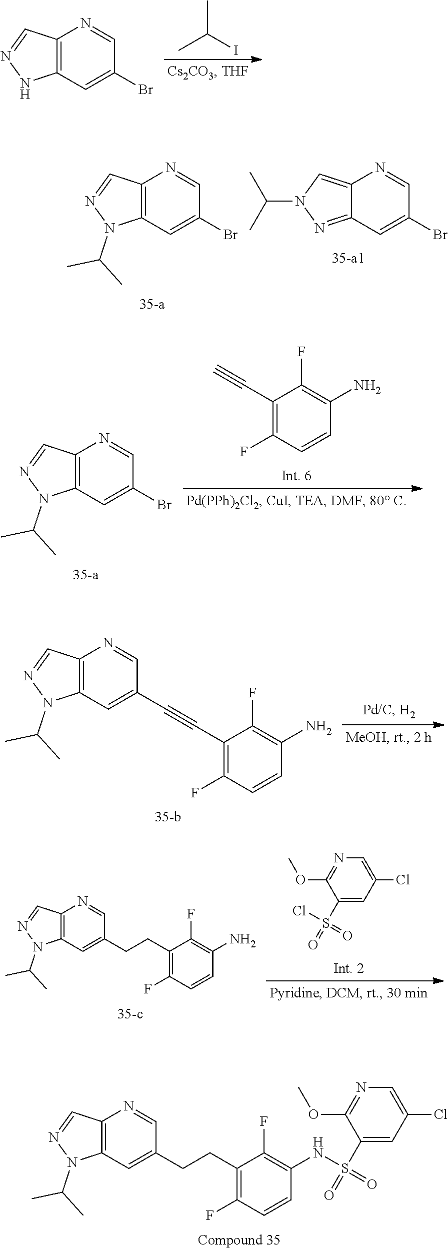

### Synthesis of 35-a: 6-bromo-1-isopropylpyrazolo[4,3-b]pyridine and 6-bromo-2-isopropyl-2H-pyrazolo[4,3-b]pyridine

Into a 50 mL round-bottom flask was placed 6-bromo-1H-pyrazolo[4,3-b]pyridine (1 g, 5 mmol, 1 equiv), THE (25 mL), Cs₂CO₃ (3.3 g, 10 mmol, 2 equiv) and 2-iodopropane (1 g, 6 mmol, 1.2 equiv). The resulting solution was stirred for 12 h at 60° C. in an oil bath, then cooled and concentrated under vacuum. The residue was purified by silica gel column chromatography, eluting with PE:EA (3:1) to afford 6-bromo-1-isopropylpyrazolo[4,3-b]pyridine (600 mg, 50% yield) and 6-bromo-2-isopropyl-2H-pyrazolo[4,3-b]pyridine (450 mg) as light brown solids.

LCMS (ES, m/z): [M+H]⁺: 240

### Synthesis of 35-b: 2,4-difluoro-3-(2-[1-isopropylpyrazolo[4,3-b]pyridin-6-yl]ethynyl)aniline

Into a 50 mL round-bottom flask was placed 6-bromo-1-isopropylpyrazolo[4,3-b]pyridine (600 mg, 2.5 mmol, 1 equiv), DMF (30 mL), CuI (47 mg, 0.25 mmol, 0.1 equiv), Pd(PPh₃)₂Cl₂ (175 mg, 0.25 mmol, 0.1 equiv), TEA (758 mg, 7.5 mmol, 3 equiv) and 3-ethynyl-2,4-difluoroaniline (765 mg, 5 mmol, 2 equiv) in portions at room temperature under N₂ atmosphere. The reaction was stirred for 2 h at 80° C. in an oil bath, then cooled and diluted with 10 mL of H₂O. The resulting solution was extracted with 2×10 mL of ethyl acetate and the organics combined, washed with 3×10 mL of brine, dried over anhydrous sodium sulfate and concentrated. The residue was purified by prep-HPLC with the following conditions: Column, WelFlash TM C18-I, Spherical C18 20-40 μm, 120 g; mobile phase: 30-80% MeCN/0.1% aqueous formic acid; to afford 2,4-difluoro-3-(2-[1-isopropylpyrazolo[4,3-b]pyridin-6-yl]ethynyl)aniline (570 mg, 73% yield) as a white solid.

LCMS (ES, m/z): [M+H]⁺: 313

### Synthesis of 35-c: 2,4-difluoro-3-(2-[1-isopropylpyrazolo[4,3-b]pyridin-6-yl]ethyl)aniline

Into a 30 mL sealed tube was placed 2,4-difluoro-3-(2-[1-isopropylpyrazolo[4,3-b]pyridin-6-yl]ethynyl)aniline (560 mg, 1.8 mmol, 1 equiv), MeOH (20 mL) and 10% Pd/C (300 mg) under H₂ (20 atm.). The reaction was stirred for 2 h at room temperature, then filtered and the filtrate concentrated. Crude 2,4-difluoro-3-(2-[1-isopropylpyrazolo[4,3-b]pyridin-6-yl]ethyl)aniline (470 mg) was isolated as a white solid which was used in next step directly without further purification.

LCMS (ES, m/z): [M+H]⁺: 317

### Synthesis of Compound 35: 5-chloro-N-[2,4-difluoro-3-(2-[1-isopropylpyrazolo[4,3-b]pyridin-6-yl]ethyl)phenyl]-2-methoxypyridine-3-sulfonamide

Into a 40 mL vial was placed 2,4-difluoro-3-(2-[1-isopropylpyrazolo[4,3-b]pyridin-6-yl]ethyl)aniline (100 mg, 0.3 mmol, 1 equiv), pyridine (5 mL), DCM (0.1 mL) and 5-chloro-2-methoxypyridine-3-sulfonyl chloride (115 mg, 0.5 mmol, 1.5 equiv). The reaction was stirred for 30 min then concentrated under vacuum. The residue was purified by prep-HPLC with the following conditions: Column, welch Vltimate XB—C18, 50×250 mm, 10 μm; Mobile Phase: 30-65% MeCN/0.1% aqueous formic acid; to afford 5-chloro-N-[2,4-difluoro-3-(2-[1-isopropylpyrazolo[4,3-b]pyridin-6-yl]ethyl)phenyl]-2-methoxypyridine-3-sulfonamide (30 mg, 18% yield) as a white solid.

LCMS (ES, m/z): [M+H]⁺: 522
¹H NMR (300 MHz, DMSO-d₆) δ 10.24 (s, 1H), 8.49 (d, J=2.6 Hz, 1H), 8.21 (dd, J=8.9, 1.3 Hz, 2H), 7.99 (d, J=2.6 Hz, 1H), 7.87 (dd, J=1.9, 1.0 Hz, 1H), 7.12 (td, J=8.9, 6.0 Hz, 1H), 6.96 (td, J=9.0, 1.6 Hz, 1H), 4.94 (h, J=6.7 Hz, 1H), 3.94 (s, 3H), 2.94 (s, 4H), 1.45 (d, J=6.7 Hz, 6H).

### Example 47: Synthesis of N-[2,4-difluoro-3-(2-[1-isoproyloyrazolo[4,3-b]pyridin-6-yl]ethyl)phenyl]-5-fluoro-2-methoxypyridine-3-sulfonamide (Compound 36)

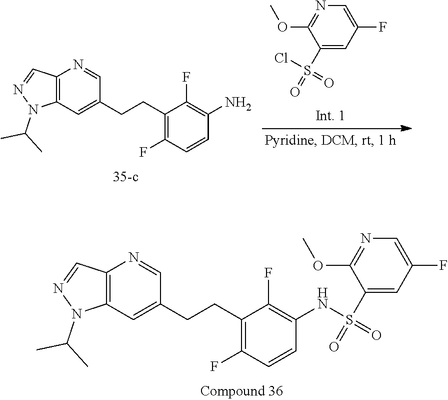 [SMILES: CC(C)n1ncc2ncc(CCc3c(F)ccc(N)c3F)cc21 ; COc1ncc(F)cc1S(=O)(=O)Cl ; COc1ncc(F)cc1S(=O)(=O)Nc1ccc(F)c(CCc2cnc3cnn(C(C)C)c3c2)c1F]

### Synthesis of Compound 36: N-[2,4-difluoro-3-(2-[1-isopropylpyrazolo[4,3-b]pyridin-6-yl]ethyl)phenyl]-5-fluoro-2-methoxypyridine-3-sulfonamide

Into a 40 mL vial was placed 2,4-difluoro-3-(2-[1-isopropylpyrazolo[4,3-b]pyridin-6-yl]ethyl)aniline (100 mg, 0.3 mmol, 1 equiv), pyridine (4 mL), DCM (0.1 mL) and 5-fluoro-2-methoxypyridine-3-sulfonyl chloride (107 mg, 0.5 mmol, 1.5 equiv). The resulting solution was stirred for 1 h then concentrated. The residue was purified by prep-HPLC with the following conditions: Column, welch Vltimate XB—C18, 50×250 mm, 10 μm; Mobile Phase: 25-60% MeCN/0.1% aqueous formic acid; to afford N-[2,4-difluoro-3-(2-[1-isopropylpyrazolo[4,3-b]pyridin-6-yl]ethyl)phenyl]-5-fluoro-2-methoxypyridine-3-sulfonamide (30 mg, 19% yield) as a white solid.

LCMS (ES, m/z): [M+H]⁺: 506
¹H NMR (300 MHz, DMSO-d₆) δ 10.21 (s, 1H), 8.45 (d, J=3.0 Hz, 1H), 8.25-8.11 (m, 2H), 7.97-7.84 (m, 2H), 7.11 (td, J=8.9, 6.0 Hz, 1H), 6.95 (t, J=8.9 Hz, 1H), 4.92 (p, J=6.6 Hz, 1H), 3.92 (s, 3H), 2.94 (s, 4H), 1.45 (d, J=6.6 Hz, 6H).

### Example 48: Synthesis of N-[2,4-difluoro-3-(2-[2-isopropylpyrazolo[4,3-b]pyridin-6-yl]ethyl)phenyl]-5-fluoro-2-methoxypyridine-3-sulfonamide (Compound 37)

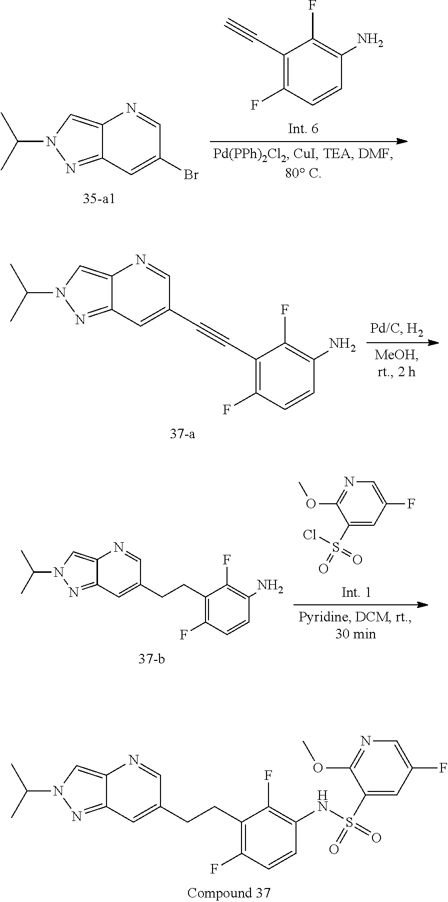 [SMILES: C#Cc1c(F)ccc(N)c1F ; CC(C)n1cc2ncc(Br)cc2n1 ; CC(C)n1cc2ncc(C#Cc3c(F)ccc(N)c3F)cc2n1 ; CC(C)n1cc2ncc(CCc3c(F)ccc(N)c3F)cc2n1 ; COc1ncc(F)cc1S(=O)(=O)Cl ; COc1ncc(F)cc1S(=O)(=O)Nc1ccc(F)c(CCc2cnc3cn(C(C)C)nc3c2)c1F]

### Synthesis of 37-a: 2,4-difluoro-3-(2-[2-isopropylpyrazolo[4,3-b]pyridin-6-yl]ethynyl)aniline

Into a 50 mL round-bottom flask was placed 6-bromo-2-isopropylpyrazolo[4,3-b]pyridine (410 mg, 1.7 mmol, 1 equiv), DMF (10 mL), CuI (33 mg, 0.17 mmol, 0.1 equiv), Pd(PPh₃)₂Cl₂ (120 mg, 0.17 mmol, 0.1 equiv), TEA (518 mg, 5 mmol, 3 equiv) and 3-ethynyl-2,4-difluoroaniline (392. mg, 2.6 mmol, 1.5 equiv) at room temperature under N₂ atmosphere. The reaction was stirred for 2 h at 80° C. then cooled and diluted with 10 mL of H₂O. The resulting solution was extracted with 2×10 mL of ethyl acetate and the organics combined, washed with 3×10 mL of brine, dried over anhydrous sodium sulfate and concentrated. The residue was purified by prep-HPLC Flash-Prep-HPLC with the following conditions: Column, WelFlash TM C18-I, Spherical C18 20-40 μm, 120 g; mobile phase: 30-80% MeCN/0.1% aqueous formic acid; to afford 2,4-difluoro-3-(2-[2-isopropylpyrazolo[4,3-b]pyridin-6-yl]ethynyl)aniline (400 mg, 75% yield) as a white solid.

LCMS (ES, m/z): [M+H]⁺: 313

### Synthesis of 37-b: 2,4-difluoro-3-(2-[2-isopropylpyrazolo[4,3-b]pyridin-6-yl]ethyl)aniline

Into a 30 mL sealed tube was placed 2,4-difluoro-3-(2-[2-isopropylpyrazolo[4,3-b]pyridin-6-yl]ethynyl)aniline (400 mg, 1.3 mmol, 1 equiv), CH₃OH (10 mL) and 10% Pd/C (200 mg) under H₂ (20 atm.). The resulting solution was stirred for 2 h then filtered through celite. The filtrate was concentrated to give 2,4-difluoro-3-(2-[2-isopropylpyrazolo[4,3-b]pyridin-6-yl]ethyl)aniline (350 mg, 86% yield) as a white solid which was used in next step directly without further purification.

LCMS (ES, m/z): [M+H]⁺: 317

### Synthesis of Compound 37: N-[2,4-difluoro-3-(2-[2-isopropylpyrazolo[4,3-b]pyridin-6-yl]ethyl)phenyl]-5-fluoro-2-methoxypyridine-3-sulfonamide

Into a 40 mL vial was placed 2,4-difluoro-3-(2-[2-isopropylpyrazolo[4,3-b]pyridin-6-yl]ethyl)aniline (110 mg, 0.3 mmol, 1 equiv), pyridine (8 mL) and 5-fluoro-2-methoxypyridine-3-sulfonyl chloride (108 mg, 0.6 mmol, 1.5 equiv). The reaction was stirred for 30 min then concentrated. The residue was purified by prep-HPLC with the following conditions: Column, welch Vltimate XB—C18, 50×250 mm, 10 μm; Mobile Phase: 20-50% MeCN/0.1% aqueous formic acid; to afford N-[2,4-difluoro-3-(2-[2-isopropylpyrazolo[4,3-b]pyridin-6-yl]ethyl)phenyl]-5-fluoro-2-methoxypyridine-3-sulfonamide (30 mg, 17% yield) as a white solid.

LCMS (ES, m/z): [M+H]⁺: 506
¹H NMR (300 MHz, DMSO-d₆) δ 10.22 (s, 1H), 8.62 (d, J=2.8 Hz, 1H), 8.45 (t, J=3.2 Hz, 1H), 8.20 (q, J=1.9 Hz, 1H), 7.93 (dt, J=6.8, 3.2 Hz, 1H), 7.67 (s, 1H), 7.08 (td, J=7.6, 6.7, 3.5 Hz, 1H), 6.99-6.87 (m, 1H), 4.88-4.77 (m, 1H), 3.97-3.90 (m, 3H), 2.94-2.83 (m, 4H), 1.65-1.51 (m, 6H).

### Example 49: Synthesis of N-[2,4-difluoro-3-[([3-methyl-1H-pyrazolo[3,4-b]pyridin-5-yl]oxy)methyl]phenyl]-5-fluoro-2-methylpyridine-3-sulfonamide (Compound 38)

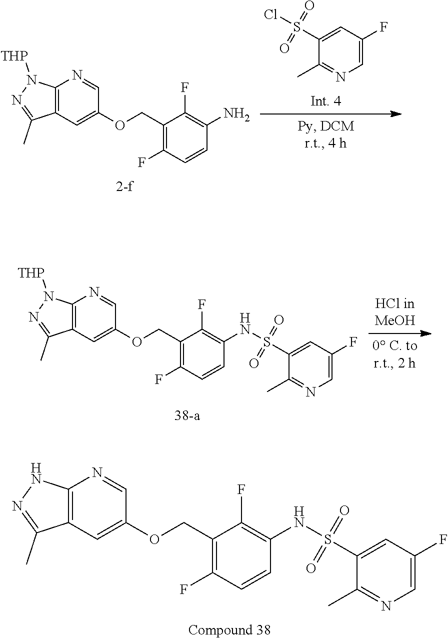

### Synthesis of 38-a: N-[2,4-difluoro-3-([[3-methyl-1-(oxan-2-yl)pyrazolo[3,4-b]pyridin-5-yl]oxy]methyl)phenyl]-5-fluoro-2-methoxypyridine-3-sulfonamide

To a stirred solution of 2,4-difluoro-3-([[3-methyl-1-(oxan-2-yl)pyrazolo[3,4-b]pyridin-5-yl]oxy]methyl)aniline (100 mg, 0.26 mmol, 1 equiv) and pyridine (85 mg, 1.1 mmol, 4 equiv) in DCM (2 mL) was added 5-fluoro-2-methylpyridine-3-sulfonyl chloride (84 mg, 0.4 mmol, 1.5 equiv) at room temperature. The reaction was stirred for 4 h and diluted with water (2 mL). The resulting mixture was extracted with DCM (3×10 mL), and the combined organics washed with water (2×10 mL), brine (10 mL), dried over anhydrous Na₂SO₄ and concentrated. The residue was purified by silica gel column chromatography, eluting with EtOAc/PE (1:1) to afford N-[2,4-difluoro-3-([[3-methyl-1-(oxan-2-yl)pyrazolo[3,4-b]pyridin-5-yl]oxy]methyl)phenyl]-5-fluoro-2-methoxypyridine-3-sulfonamide (110 mg, 73% yield) as a yellow solid.

LCMS (ES, m/z): [M+H]⁺: 548.

### Synthesis of Compound 38: N-[2,4-difluoro-3-[([3-methyl-1H-pyrazolo[3,4-b]pyridin-5-yl]oxy)methyl]phenyl]-5-fluoro-2-methylpyridine-3-sulfonamide

N-[2,4-difluoro-3-([[3-methyl-1-(oxan-2-yl)pyrazolo[3,4-b]pyridin-5-yl]oxy]methyl)phenyl]-5-fluoro-2-methylpyridine-3-sulfonamide (90 mg, 0.16 mmol, 1 equiv) was added to 4 M HCl in MeOH (1 mL) at 0° C. and the resulting mixture warmed to room temperature over 2 h. The resulting mixture was concentrated under vacuum, and the residue was neutralized to pH 7 with NH₃·H₂O (5%). This was purified by Prep-HPLC with the following conditions: Column: welch Vltimate XB—C18, 50×250 mm, 10 m; mobile phase: 90 mL/min, 20-50% MeCN/0.1% aqueous formic acid; to afford N-[2,4-difluoro-3-[([3-methyl-1H-pyrazolo[3,4-b]pyridin-5-yl]oxy)methyl]phenyl]-5-fluoro-2-methylpyridine-3-sulfonamide (37 mg, 48% yield) as a white solid.

LCMS (ES, m/z): [M+H]⁺: 464.
¹H NMR (300 MHz, DMSO-d₆) δ 13.09 (s, 1H), 10.72 (s, 1H), 8.69 (d, J=2.8 Hz, 1H), 8.16 (d, J=2.7 Hz, 1H), 7.93-7.80 (m, 2H), 7.37 (td, J=8.9, 5.9 Hz, 1H), 7.16 (t, J=9.0 Hz, 1H), 5.11 (s, 2H), 2.76 (d, J=1.2 Hz, 3H), 2.48 (s, 3H).

### Example 50: Synthesis of N-[3-[([3-ethyl-1H-Pyrazolo[3,4-b]pyridin-5-yl]oxy)methyl]-2,4-difluorophenyl]-5-fluoro-2-methoxypyridine-3-sulfonamide (Compound 39)

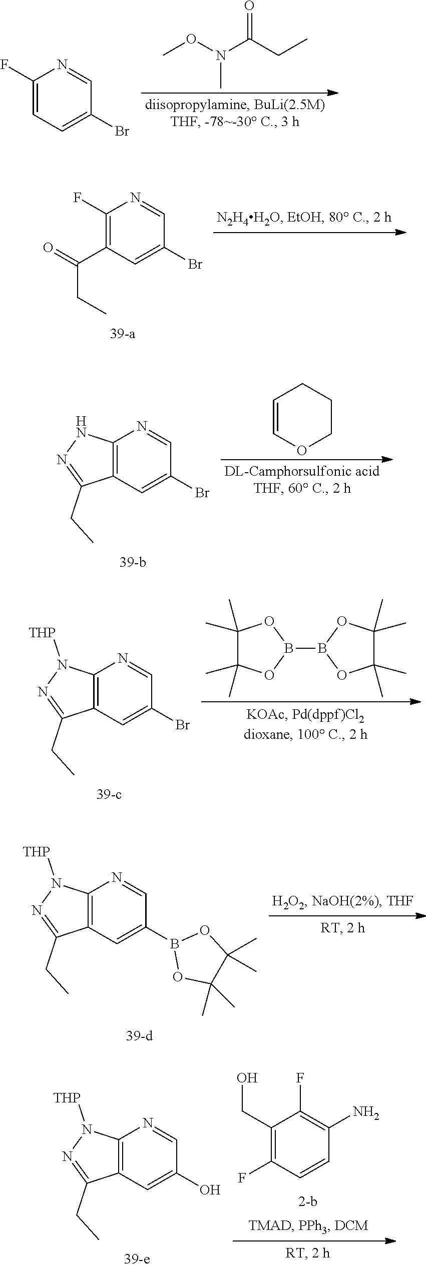

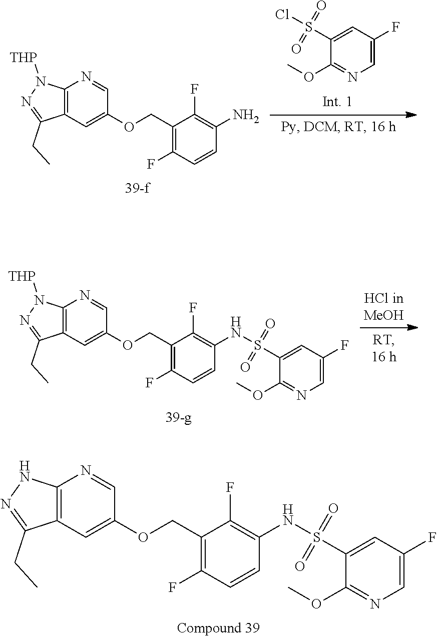

### Synthesis of 39-a: 1-(5-bromo-2-fluoropyridin-3-yl)propan-1-one

To a stirred solution of diisopropylamine (15 g, 148 mmol, 1.3 equiv) in THF (300 mL) was added 2.5 M butyllithium in hexanes (59 mL, 148 mmol, 1.3 equiv) dropwise at −78° C. under nitrogen atmosphere. The LDA solution was stirred for 0.5 h at −78° C., then 5-bromo-2-fluoropyridine (20 g, 114 mmol, 1 equiv) was added dropwise over 10 min at −78° C. The resulting mixture was stirred for 1 h at −65° C., then N-methoxy-N-methylpropanamide (14.6 g, 125 mmol, 1.1 equiv) in THF (20 mL) was added dropwise over 10 min at −78° C. The solution was stirred for 1 hr at −30° C., before being quenched with sat. NH₄Cl (aq.) at low temperature. The resulting mixture was extracted with EtOAc (3×300 mL), and the combined organics washed with brine (500 mL), dried over anhydrous Na₂SO₄ and concentrated under reduced pressure. The residue was purified by silica gel column chromatography, eluting with PE/EtOAc (8:1) to afford 1-(5-bromo-2-fluoropyridin-3-yl)propan-1-one (25 g, 95% yield) as a light yellow oil.

LCMS (ES, m/z): [M+H]⁺: 232

### Synthesis of 39-b: 5-bromo-3-ethyl-1H-pyrazolo[3,4-b]pyridine

To a stirred solution of 1-(5-bromo-2-fluoropyridin-3-yl)propan-1-one (25 g, 108 mmol, 1 equiv) in EtOH (500 mL) was added NH₂NH₂·H₂O (16.2 g, 323 mmol, 3 equiv) dropwise. The reaction was stirred for 16 h at 80° C. under nitrogen atmosphere, then cooled and concentrated under reduced pressure. The residue was purified by silica gel column chromatography, eluting with PE/EtOAc (6:1) to afford 5-bromo-3-ethyl-1H-pyrazolo[3,4-b]pyridine (12 g, 49% yield) as a light yellow oil.

LCMS (ES, m/z): [M+H]⁺: 226.

### Synthesis of 39-c: 5-bromo-3-ethyl-1-(oxan-2-yl)pyrazolo[3,4-b]pyridine

To a stirred solution of 5-bromo-3-ethyl-1H-pyrazolo[3,4-b]pyridine (12 g, 53 mmol, 1 equiv) and dihydropyran (22 g, 265 mmol, 5 equiv) in THE (200 mL) was added DL-Camphor sulfonic acid (1.85 g, 8 mmol, 0.15 equiv). The reaction mixture was stirred for 2 h at 60° C. then concentrated under reduced pressure. The residue was purified by silica gel column chromatography, eluting with PE/EtOAc (8:1) to afford 5-bromo-3-ethyl-1-(oxan-2-yl)pyrazolo[3,4-b]pyridine (11 g, 67% yield) as a light yellow oil.

LCMS (ES, m/z): [M+H]⁺: 310.

### Synthesis of 39-d: 3-ethyl-1-(oxan-2-yl)-5-(4,4,5,5-tetramethyl-1,3,2-dioxaborolan-2-yl) pyrazolo[3,4-b]pyridine

To a solution of 5-bromo-3-ethyl-1-(oxan-2-yl)pyrazolo[3,4-b]pyridine (10 g, 32 mmol, 1 equiv) and bis(pinacolato)diboron (12 g, 48 mmol, 1.5 equiv) in dioxane (150 mL) were added KOAc (6.3 g, 64 mmol, 2 equiv) and Pd(dppf)Cl₂ (1.32 g, 1.6 mmol, 0.05 equiv). After stirring for 2 h at 100° C., the resulting mixture was concentrated under reduced pressure. The residue was purified by silica gel column chromatography, eluting with PE/EtOAc (8:1) to afford 3-ethyl-1-(oxan-2-yl)-5-(4,4,5,5-tetramethyl-1,3,2-dioxaborolan-2-yl)pyrazolo[3,4-b]pyridine (10 g, 87% yield) as a light yellow oil.

LCMS (ES, m/z): [M+H]⁺: 358.

### Synthesis of 39-e: 3-ethyl-1-(oxan-2-yl)pyrazolo[3,4-b]pyridin-5-ol

To a stirred solution of 3-ethyl-1-(oxan-2-yl)-5-(4,4,5,5-tetramethyl-1,3,2-dioxaborolan-2-yl)pyrazolo[3,4-b]pyridine (2.6 g, 7.3 mmol, 1 equiv) in THE (20 mL) and NaOH (20 mL, 2% aq.) was added 30% H₂O₂ (8.2 g, 73 mmol, 10 eq) dropwise. The reaction mixture was stirred for 2 h then quenched with sat. Na₂S₂O₃ (aq.). The resulting mixture was extracted with EtOAc (2×50 mL), and the combined organics were washed with brine (50 mL), dried over anhydrous Na₂SO₄ and concentrated. The residue was purified by silica gel column chromatography, eluting with PE/EtOAc (4:1) to afford 3-ethyl-1-(oxan-2-yl)pyrazolo[3,4-b]pyridin-5-ol (1.2 g, 67% yield) as a light yellow oil.

LCMS (ES, m/z): [M+H]⁺: 248.

### Synthesis of 39-f: 3-([[3-ethyl-1-(oxan-2-yl)pyrazolo[3,4-b]pyridin-5-yl]oxy]methyl)-2,4-difluoroaniline

To a stirred solution of 3-ethyl-1-(oxan-2-yl)pyrazolo[3,4-b]pyridin-5-ol (1.2 g, 4.8 mmol, 1 equiv), (3-amino-2,6-difluorophenyl)methanol (0.93 g, 5.8 mmol, 1.2 equiv) and PPh₃ (1.9 g, 7.3 mmol, 1.5 equiv) in DCM (30 mL) was added TMAD (1.25 g, 7.3 mmol, 1.5 equiv). The reaction was stirred for 2 h then diluted with water (50 mL) and extracted with DCM (2×50 mL). The combined organics were washed with brine (50 mL), dried over anhydrous Na₂SO₄ and concentrated. The residue was purified by silica gel column chromatography, eluting with PE/EtOAc (2:1) to afford 3-([[3-ethyl-1-(oxan-2-yl)pyrazolo[3,4-b]pyridin-5-yl]oxy]methyl)-2,4-difluoroaniline (1.1 g, 58% yield) as a light yellow solid.

LCMS (ES, m/z): [M+H]⁺: 389.

### Synthesis of 39-g: N-[3-([[3-ethyl-1-(oxan-2-yl)pyrazolo[3,4-b]pyridin-5-yl]oxy]methyl)-2,4-difluorophenyl]-5-fluoro-2-methoxypyridine-3-sulfonamide

To a stirred solution of 3-([[3-ethyl-1-(oxan-2-yl)pyrazolo[3,4-b]pyridin-5-yl]oxy]methyl)-2,4-difluoroaniline (100 mg, 0.26 mmol, 1 equiv) and pyridine (81 mg, 1 mmol, 4 equiv) in DCM (3 mL) was added 5-fluoro-2-methoxypyridine-3-sulfonylchloride (75 mg, 0.33 mmol, 1.3 equiv) in DCM (0.5 mL) dropwise. The resulting mixture was stirred for 16 h then diluted with water (10 mL). This was extracted with DCM (3×10 mL), and the combined organics were washed with brine (20 mL), dried over anhydrous Na₂SO₄ and concentrated. The residue was purified by silica gel column chromatography, eluting with PE/EtOAc (2:1) to afford N-[3-([[3-ethyl-1-(oxan-2-yl)pyrazolo[3,4-b]pyridin-5-yl]oxy]methyl)-2,4-difluorophenyl]-5-fluoro-2-methoxypyridine-3-sulfonamide (130 mg, 87% yield) as a white solid.

### Synthesis of Compound 39: N-[3-[([3-ethyl-1H-pyrazolo[3,4-b]pyridin-5-yl]oxy)methyl]-2,4-difluorophenyl]-5-fluoro-2-methoxypyridine-3-sulfonamide

A mixture of N-[3-([[3-ethyl-1-(oxan-2-yl)pyrazolo[3,4-b]pyridin-5-yl]oxy]methyl)-2,4-difluorophenyl]-5-fluoro-2-methoxypyridine-3-sulfonamide (110 mg, 0.19 mmol, 1 equiv) in 4 M HCl in MeOH (2 mL) was stirred for 2 h then diluted with water (10 mL). The resulting mixture was extracted with EtOAc (2×10 mL). The combined organics were dried (MgSO₄), concentrated under reduced pressure and the residue purified by Prep-HPLC with the following conditions: Column: XbridgeC18, 19*150 mm, 5 m; mobile phase: 20 mL/min, 2-27% MeCN/0.05% aqueous ammonia; to afford N-[3-[([3-ethyl-1H-pyrazolo[3,4-b]pyridin-5-yl]oxy)methyl]-2,4-difluorophenyl]-5-fluoro-2-methoxypyridine-3-sulfonamide (55 mg, 59% yield) as a white solid.

LCMS (ES, m/z): [M+H]⁺: 494.
¹H NMR (300 MHz, DMSO-d₆) δ 13.09 (s, 1H), 10.36 (s, 1H), 8.44 (d, J=2.9 Hz, 1H), 8.17 (d, J=2.7 Hz, 1H), 7.97 (dd, J=7.3, 3.0 Hz, 1H), 7.87 (d, J=2.8 Hz, 1H), 7.44-7.30 (m, 1H), 7.21-7.09 (m, 1H), 5.14 (s, 2H), 3.90 (s, 3H), 2.90 (q, J=7.6 Hz, 2H), 1.32 (t, J=7.6 Hz, 3H).

### Example 51: Synthesis of 5-chloro-N-[3-[([3-ethyl-1H-pyrazolo[3,4-b]pyridin-5-yl]oxy)methyl]-2,4-difluorophenyl]-2-methoxypyridine-3-sulfonamide (Compound 40)

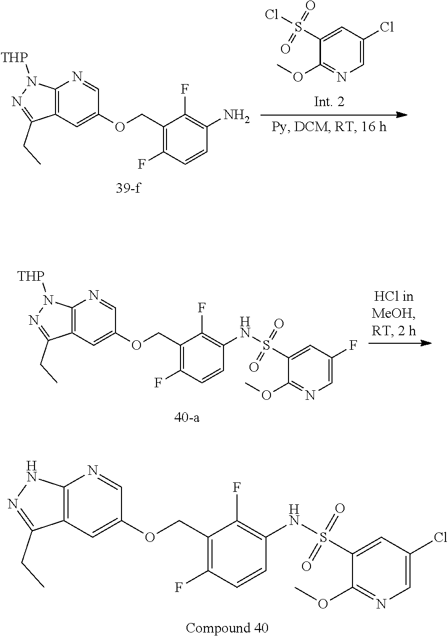

### Synthesis of 40-a: 5-chloro-N-[3-([[3-ethyl-1-(oxan-2-yl)pyrazolo[3,4-b]pyridin-5-yl]oxy]methyl)-2,4-difluorophenyl]-2-methoxypyridine-3-sulfonamide

To a stirred solution of 3-([[3-ethyl-1-(oxan-2-yl)pyrazolo[3,4-b]pyridin-5-yl]oxy]methyl)-2,4-difluoroaniline (100 mg, 0.26 mmol, 1 equiv) and pyridine (82 mg, 1.1 mmol, 4 equiv) in DCM (3 mL) was added 5-chloro-2-methoxypyridine-3-sulfonyl chloride (81 mg, 0.3 mmol, 1.3 equiv) in DCM (0.5 mL) dropwise. The resulting mixture was stirred for 16 h at room temperature then diluted with water (10 mL). This was extracted with DCM (3×10 mL), and the combined organics washed with brine (20 mL), dried over anhydrous Na₂SO₄ and concentrated. The residue was purified by silica gel column chromatography, eluting with PE/EtOAc (2:1) to afford 5-chloro-N-[3-([[3-ethyl-1-(oxan-2-yl)pyrazolo[3,4-b]pyridin-5-yl]oxy]methyl)-2,4-difluorophenyl]-2-methoxypyridine-3-sulfonamide (130 mg, 85% yield) as a white solid.

LCMS (ES, m/z): [M+H]⁺: 594.

### Synthesis of Compound 40: 5-chloro-N-[3-[([3-ethyl-1H-pyrazolo[3,4-b]pyridin-5-yl]oxy)methyl]-2,4-difluorophenyl]-2-methoxypyridine-3-sulfonamide

A mixture of 5-chloro-N-[3-([[3-ethyl-1-(oxan-2-yl)pyrazolo[3,4-b]pyridin-5-yl]oxy]methyl)-2,4-difluorophenyl]-2-methoxypyridine-3-sulfonamide (80 mg, 0.13 mmol, 1 equiv) in 4 M HCl in MeOH (1.5 mL) was stirred for 2 h at room temperature then concentrated. The solid formed was washed with MeCN (2×3 mL) and dried to give 5-chloro-N-[3-[([3-ethyl-1H-pyrazolo[3,4-b]pyridin-5-yl]oxy)methyl]-2,4-difluorophenyl]-2-methoxypyridine-3-sulfonamide (31 mg, 46% yield) as a white solid.

LCMS (ES, m/z): [M+H]⁺: 510.
¹H NMR (300 MHz, DMSO-d₆) δ 13.09 (s, 1H), 10.38 (s, 1H), 8.48 (d, J=2.6 Hz, 1H), 8.18 (d, J=2.7 Hz, 1H), 8.03 (d, J=2.6 Hz, 1H), 7.87 (d, J=2.7 Hz, 1H), 7.38 (td, J=8.9, 6.0 Hz, 1H), 7.23-7.11 (m, 1H), 5.15 (s, 2H), 3.92 (s, 3H), 2.90 (q, J=7.6 Hz, 2H), 1.32 (t, J=7.6 Hz, 3H).

### Example 52: Synthesis of 5-cyano-N-[3-[([3-ethyl-1H-pyrazolo[3,4-b]pyridin-5-yl]oxy)methyl]-2,4-difluorophenyl]-2-methoxypyridine-3-sulfonamide (Compound 41)

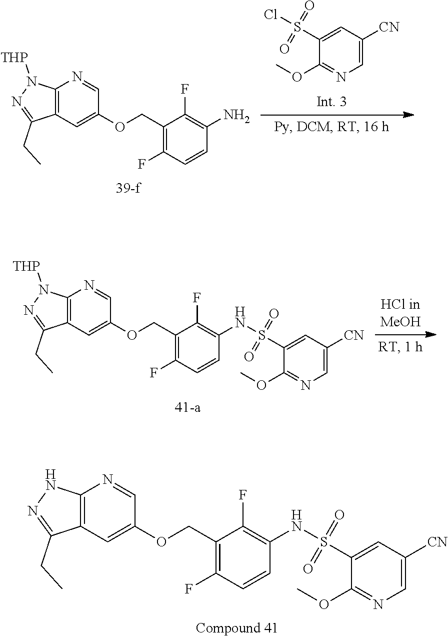

### Synthesis of 41-a: 5-cyano-N-[3-([[3-ethyl-1-(oxan-2-yl)pyrazolo[3,4-b]pyridin-5-yl]oxy]methyl)-2,4-difluorophenyl]-2-methoxypyridine-3-sulfonamide

To a stirred solution of 3-([[3-ethyl-1-(oxan-2-yl)pyrazolo[3,4-b]pyridin-5-yl]oxy]methyl)-2,4-difluoroaniline (100 mg, 0.26 mmol, 1 equiv) and pyridine (82 mg, 1.1 mmol, 4 equiv) in DCM (3 mL) was added 5-cyano-2-methoxypyridine-3-sulfonyl chloride (78 mg, 0.36 mmol, 1.3 equiv) in DCM (0.5 mL). The reaction was stirred for 16 h then diluted with water (10 mL). The resulting mixture was extracted with DCM (3×10 mL), and the combined organics washed with brine (20 mL), dried over anhydrous Na₂SO₄ and concentrated. The residue was purified by silica gel column chromatography, eluting with PE/EtOAc (2:1) to afford 5-cyano-N-[3-([[3-ethyl-1-(oxan-2-yl)pyrazolo[3,4-b]pyridin-5-yl]oxy]methyl)-2,4-difluorophenyl]-2-methoxypyridine-3-sulfonamide (140 mg, 93% yield) as a white solid.

LCMS (ES, m/z): [M+H]⁺:585.

### Synthesis of Compound 41: 5-cyano-N-[3-[([3-ethyl-1H-pyrazolo[3,4-b]pyridin-5-yl]oxy)methyl]-2,4-difluorophenyl]-2-methoxypyridine-3-sulfonamide

A mixture of 5-cyano-N-[3-([[3-ethyl-1-(oxan-2-yl)pyrazolo[3,4-b]pyridin-5-yl]oxy]methyl)-2,4-difluorophenyl]-2-methoxypyridine-3-sulfonamide (130 mg, 0.22 mmol, 1 equiv) in 4 M HCl in MeOH (2 mL) was stirred for 2 h then diluted with water (10 mL). The resulting mixture was extracted with EtOAc (3×10 mL), and the combined organics concentrated. The crude product was purified by Prep-HPLC with the following conditions: Column: XbridgeC18, 19*150 mm, 5 μm; Flowrate 20 mL/min, Mobile Phase: 14-36% MeCN/0.05% aqueous ammonia; to afford 5-cyano-N-[3-[([3-ethyl-1H-pyrazolo[3,4-b]pyridin-5-yl]oxy)methyl]-2,4-difluorophenyl]-2-methoxypyridine-3-sulfonamide (45 mg, 41% yield) as a white solid.

LCMS (ES, m/z): [M+H]⁺: 501.
¹H NMR (300 MHz, DMSO-d₆) δ 13.08 (s, 1H), 8.86 (d, J=2.2 Hz, 1H), 8.42 (d, J=2.3 Hz, 1H), 8.19 (d, J=2.7 Hz, 1H), 7.88 (d, J=2.7 Hz, 1H), 7.42-7.28 (m, 1H), 7.08 (t, J=8.9 Hz, 1H), 5.13 (s, 2H), 3.98 (s, 3H), 2.90 (q, J=7.6 Hz, 2H), 1.32 (t, J=7.6 Hz, 3H).

### Example 53: Synthesis of 5-chloro-N-[2,4-difluoro-3-[([1-methylpyrazolo[4,3-b]pyridin-6-yl]oxy)methyl]phenyl]-2-methoxypyridine-3-sulfonamide (Compound 42)

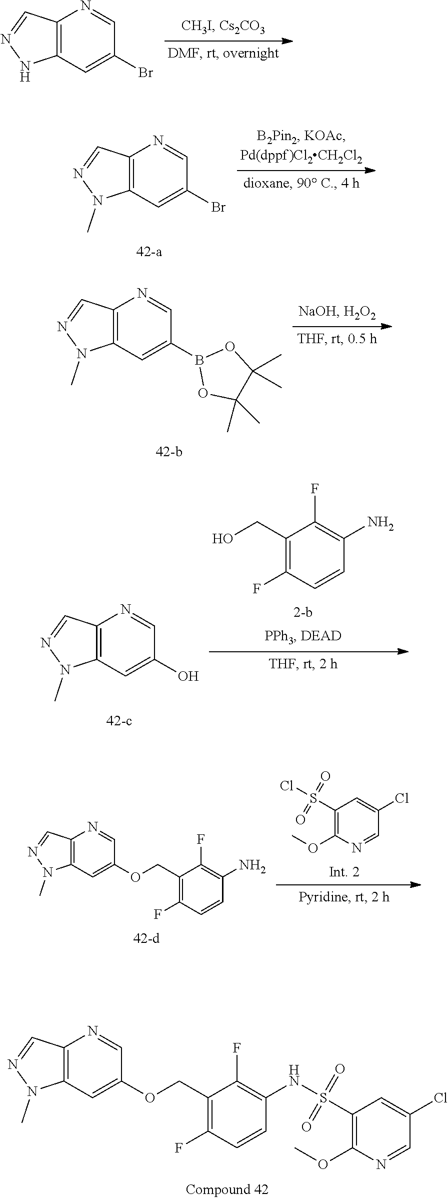

### Synthesis of 42-a: 6-bromo-1-methylpyrazolo[4,3-b]pyridine

To a stirred solution of 6-bromo-1H-pyrazolo[4,3-b]pyridine (700 mg, 3.5 mmol, 1 equiv) and Cs₂CO₃ (2.3 g, 7 mmol, 2 equiv) in DMF (7 mL) was added methyl iodide (552 mg, 3.9 mmol, 1.1 equiv) dropwise at room temperature. The resulting solution was stirred overnight, then quenched with water (20 mL). The mixture was extracted with EA (3×10 mL), and the combined organics washed with brine (2×5 mL), dried over anhydrous Na₂SO₄ and concentrated. The residue was purified by silica gel column chromatography, eluting with PE:EA (3:1) to afford 6-bromo-1-methylpyrazolo[4,3-b]pyridine (200 mg, 26% yield) as a white solid.

LCMS (ES, m/z): [M+H]⁺: 212

### Synthesis of 42-b/c: 1-methylpyrazolo[4,3-b]pyridin-6-ol

To a stirred mixture of 6-bromo-1-methylpyrazolo[4,3-b]pyridine (324 mg, 1.5 mmol, 1 equiv) and bis(pinacolato)diboron (582 mg, 2.3 mmol, 1.5 equiv) in dioxane (3 mL) were added KOAc (300 mg, 3.1 mmol, 2 equiv) and Pd(dppf)Cl₂·CH₂Cl₂ (62 mg, 0.076 mmol, 0.05 equiv) under N₂ atmosphere. The resulting mixture was stirred for 4 h at 90° C. under N₂ atmosphere, then cooled. THF (1 mL) was added, followed by NaOH (1 mL, 2%) and 30% H₂O₂ (1.73 g, 15.3 mmol, 10 equiv) at room temperature. The resulting mixture was stirred for 0.5 h then concentrated. The residue was purified by silica gel chromatography, eluting with PE:EA (3:1) to give 1-methylpyrazolo[4,3-b]pyridin-6-ol (189 mg, 82% yield) as a white solid.

LCMS (ES, m/z): [M+H]⁺: 150

### Synthesis of 42-d: 2,4-difluoro-3-[([1-methylpyrazolo[4,3-b]pyridin-6-yl]oxy)methyl]aniline

To a stirred mixture of 1-methylpyrazolo[4,3-b]pyridin-6-ol (189 mg, 1.3 mmol, 1 equiv), (3-amino-2,6-difluorophenyl)methanol (302 mg, 1.9 mmol, 1.5 equiv) and PPh₃ (500 mg, 1.9 mmol, 1.5 equiv) in THE (2 mL) was added DEAD (330 mg, 1.9 mmol, 1.5 equiv) dropwise at 0° C. The resulting mixture was stirred for 2 h then concentrated. The residue was purified by silica gel chromatography, eluting with PE:EA (1:1) to give 2,4-difluoro-3-[([1-methylpyrazolo[4,3-b]pyridin-6-yl]oxy)methyl]aniline (330 mg, 89% yield) as a white solid.

### Synthesis of Compound 42: 5-chloro-N-(2,4-difluoro-3-(((1-methyl-1H-pyrazolo[4,3-b]pyridin-6-yl)oxy)methyl)phenyl)-2-methoxypyridine-3-sulfonamide

To a stirred solution of 2,4-difluoro-3-[([1-methylpyrazolo[4,3-b]pyridin-6-yl]oxy)methyl]aniline (100 mg, 0.34 mmol, 1 equiv) in pyridine (2 mL) was added 5-chloro-2-methoxypyridine-3-sulfonyl chloride (91 mg, 0.38 mmol, 1.1 equiv) in portions at room temperature. The resulting mixture was stirred for 1 h then concentrated. The residue was purified by prep-HPLC with the following conditions: Column, welch Vltimate XB—C18, 50×250 mm, 10 μm; Mobile Phase: 15-60% MeCN/0.1% aqueous TFA; to give 5-chloro-N-(2,4-difluoro-3-(((1-methyl-1H-pyrazolo[4,3-b]pyridin-6-yl)oxy)methyl)phenyl)-2-methoxypyridine-3-sulfonamide (120 mg, 70% yield) as an off-white solid.

LCMS (ES, m/z): [M+H]⁺: 496
¹H NMR (300 MHz, DMSO-d₆) δ 10.41 (s, 1H), 8.48 (d, J=2.6 Hz, 1H), 8.18 (dd, J=9.2, 1.7 Hz, 2H), 8.04 (d, J=2.6 Hz, 1H), 7.84 (d, J=2.6 Hz, 1H), 7.42 (td, J=9.0, 6.0 Hz, 1H), 7.20 (td, J=9.0, 1.5 Hz, 1H), 5.20 (s, 2H), 4.04 (s, 3H), 3.92 (s, 3H).

### Example 54: Synthesis of N-[2,4-difluoro-3-[([1-methylpyrazolo[4,3-b]pyridin-6-yl]oxy)methyl]phenyl]-5-fluoro-2-methoxypyridine-3-sulfonamide (Compound 43)

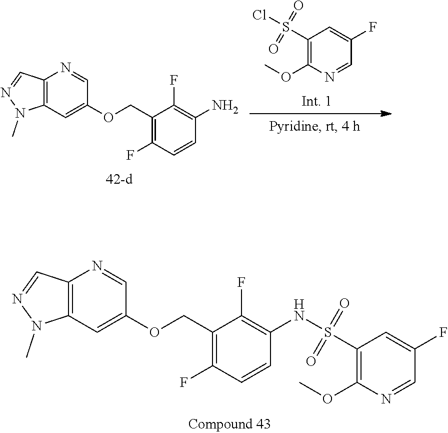 [SMILES: Cn1ncc2ncc(OCc3c(F)ccc(N)c3F)cc21 ; COc1ncc(F)cc1S(=O)(=O)Cl ; COc1ncc(F)cc1S(=O)(=O)Nc1ccc(F)c(COc2cnc3cnn(C)c3c2)c1F]

### Synthesis of Compound 43: N-[2,4-difluoro-3-[([1-methylpyrazolo[4,3-b]pyridin-6-yl]oxy)methyl]phenyl-5-fluoro-2-methoxypyridine-3-sulfonamide

To a stirred solution of 2,4-difluoro-3-[([1-methylpyrazolo[4,3-b]pyridin-6-yl]oxy)methyl]aniline (100 mg, 0.35 mmol, 1 equiv) in pyridine (4 mL) was added 5-fluoro-2-methoxypyridine-3-sulfonyl chloride (86 mg, 0.38 mmol, 1.1 equiv) in portions at room temperature. The resulting mixture was stirred for 4 h then concentrated. The residue was purified by prep-HPLC with the following conditions: Column, welch Vltimate XB—C18, 50×250 mm, 10 μm; Mobile Phase: 30-70% MeCN/0.1% aqueous TFA; to give N-[2,4-difluoro-3-[([1-methylpyrazolo[4,3-b]pyridin-6-yl]oxy)methyl]phenyl]-5-fluoro-2-methoxypyridine-3-sulfonamide (120 mg, 73% yield) as an off-white solid.

LCMS (ES, m/z): [M+H]⁺: 480
¹H NMR (300 MHz, DMSO-d₆) δ 10.40 (s, 1H), 8.45 (d, J=2.9 Hz, 1H), 8.18 (dd, J=8.8, 1.7 Hz, 2H), 7.99 (dd, J=7.3, 3.0 Hz, 1H), 7.84 (dd, J=2.6, 1.0 Hz, 1H), 7.41 (td, J=8.9, 5.9 Hz, 1H), 7.19 (td, J=9.1, 1.6 Hz, 1H), 5.19 (s, 2H), 4.04 (s, 3H), 3.91 (s, 3H).

### Example 55: Synthesis of N-[2,4-difluoro-3-([[3-(trifluoromethyl)-1H-pyrazolo[3,4-b]pyridin-5-yl]oxy]methyl)phenyl]-5-fluoro-2-methoxypyridine-3-sulfonamide (Compound 44)

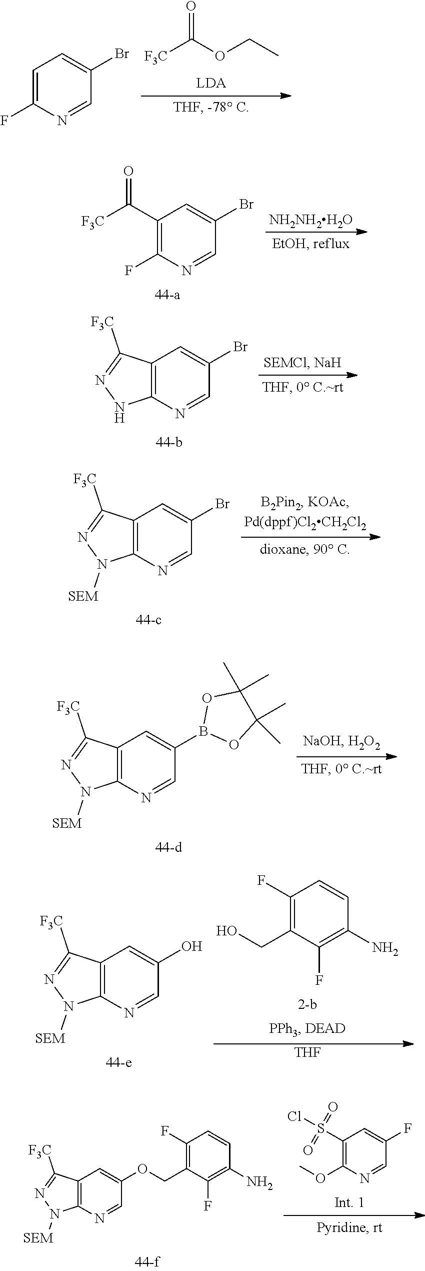

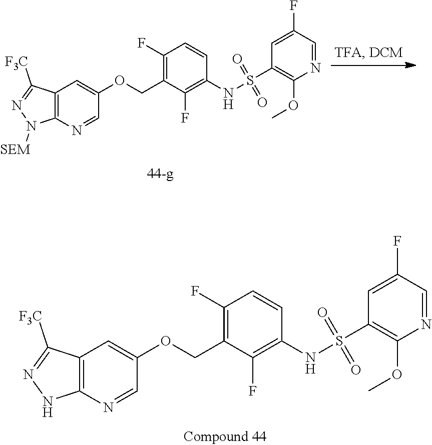 [SMILES: COc1ncc(F)cc1S(=O)(=O)Nc1ccc(F)c(COc2cnc3c(c2)c(C(F)(F)F)nn3COCC[Si](C)(C)C)c1F ; COc1ncc(F)cc1S(=O)(=O)Nc1ccc(F)c(COc2cnc3[nH]nc(C(F)(F)F)c3c2)c1F]

### Synthesis of 44-a: 1-(5-bromo-2-fluoropyridin-3-yl)-2,2,2-trifluoroethanone

To a stirred solution of 5-bromo-2-fluoropyridine (5 g, 28.4 mmol, 1 equiv) in THF (35 mL) were added 2 M LDA solution in THF (15.6 mL, 31 mmol, 1.1 equiv) dropwise at −78° C. under N₂ atmosphere. The resulting solution was stirred for 1.5 h at −78° C., then trifluoroethyl acetate (5.6 g, 40 mmol, 1.4 equiv) was added dropwise at −78° C. The cooling bath was removed and the mixture was stirred for 1 h at room temperature. The reaction mixture was quenched with 1 M aqueous HCl (10 mL) and extracted with EA (3×50 mL). The combined organics were washed with brine (2×25 mL), dried over anhydrous Na₂SO₄ and concentrated to afford 1-(5-bromo-2-fluoropyridin-3-yl)-2,2,2-trifluoroethanone (8 g, crude) as a red oil which was used in the next step directly without further purification.

LCMS (ES, m/z): [M+H]⁺: 272

### Synthesis of 44-b: 5-bromo-3-(trifluoromethyl)-1H-pyrazolo[3,4-b]pyridine

To a stirred solution of 1-(5-bromo-2-fluoropyridin-3-yl)-2,2,2-trifluoroethanone (8 g, 29 mmol, 1 equiv) in EtOH (80 mL) was added NH₂NH₂·H₂O (3 mL, 59 mmol, 2 equiv) at room temperature. The resulting mixture was stirred at reflux for 2 h, then cooled and concentrated. The residue was dissolved in EA (200 mL), washed with water (2×50 mL), brine (2×20 mL) and dried over anhydrous Na₂SO₄. Concentration afforded 5-bromo-3-(trifluoromethyl)-1H-pyrazolo[3,4-b]pyridine (7 g, crude) as a brown oil which was used in the next step directly without further purification.

LCMS (ES, m/z): [M+H]⁺: 266

### Synthesis of 44-c: 5-bromo-3-(trifluoromethyl)-1-[[2-(trimethylsilyl)ethoxy]methyl]pyrazolo[3,4-b]pyridine

To a stirred solution of 5-bromo-3-(trifluoromethyl)-1H-pyrazolo[3,4-b]pyridine (7 g, 26 mmol, 1 equiv) in THE (70 mL) was added 60% NaH in oil (2.1 g, 52.6 mmol, 2 equiv) in portions at 0° C. The resulting solution was stirred for 0.5 h at 0° C., then SEMCl (5.3 g, 31.6 mmol, 1.2 equiv) was added dropwise at 0° C. The reaction mixture was stirred for 2 h at room temperature, then quenched with water (50 mL). The resulting mixture was extracted with EA (3×50 mL), and the combined organics were washed with brine (2×50 mL), dried over anhydrous Na₂SO₄ and concentrated. The residue was purified by silica gel column chromatography, eluting with PE:EA (1:1) to afford 5-bromo-3-(trifluoromethyl)-1-[[2-(trimethylsilyl)ethoxy]methyl]pyrazolo[3,4-b]pyridine (6.5 g, 62% yield) as a light yellow oil.

LCMS (ES, m/z): [M+H]⁺: 396

### Synthesis of 44-d/e: 3-(trifluoromethyl)-1-[[2-(trimethylsilyl)ethoxy]methyl]pyrazolo[3,4-b]pyridin-5-ol

To a solution of 5-bromo-3-(trifluoromethyl)-1-[[2-(trimethylsilyl)ethoxy]methyl]pyrazolo[3,4-b]pyridine (6.5 g, 16 mmol, 1 equiv) and bis(pinacolato)diboron (6.3 g, 25 mmol, 1.5 equiv) in dioxane (70 mL) were added KOAc (3.3 g, 33 mmol, 2 equiv) and Pd(dppf)Cl₂·CH₂Cl₂ (670 mg, 0.82 mmol, 0.05 equiv). After stirring for 2 h at 90° C. under a nitrogen atmosphere, the reaction mixture was allowed to cool. THE (50 mL) was added, followed by NaOH (30 mL, 2% aqueous) at room temperature. To this was added 30% H₂O₂ (18.5 g, 164 mmol, 10 equiv) dropwise at 0° C. The resulting mixture was stirred for 0.5 h then concentrated under reduced pressure. The residue was purified by Flash-Prep-HPLC with the following conditions: Column, WelFlash TM C18-I, Spherical C18 20-40 μm, 120 g; mobile phase: 30-80% MeCN/water; to afford 3-(trifluoromethyl)-1-[[2-(trimethylsilyl)ethoxy]methyl]pyrazolo[3,4-b]pyridin-5-ol (2.3 g, 42% yield) as a white solid.

LCMS (ES, m/z): [M+H]⁺: 334

### Synthesis of 44-f: 2,4-difluoro-3-([[3-(trifluoromethyl)-1-[[2-(trimethylsilyl)ethoxy]methyl]pyrazolo[3,4-b]pyridin-5-yl]oxy]methyl)aniline

To a stirred mixture of 3-(trifluoromethyl)-1-[[2-(trimethylsilyl)ethoxy]methyl]pyrazolo[3,4-b]pyridin-5-ol (550 mg, 1.7 mmol, 1 equiv), (3-amino-2,6-difluorophenyl)methanol (315 mg, 2 mmol, 1.2 equiv) and PPh₃ (649 mg, 2.5 mmol, 1.5 equiv) in THE (10 mL) was added DEAD (430 mg, 2.5 mmol, 1.5 equiv) dropwise at 0° C. under N₂ atmosphere. The resulting solution was stirred for 2 h then quenched with water (20 mL). This was extracted with EA (3×30 mL), and the combined organics were washed with brine (2×20 mL), dried over anhydrous Na₂SO₄ and concentrated. The residue was purified by Flash-Prep-HPLC with the following conditions: Column, WelFlash TM C18-I, Spherical C18 20-40 μm, 120 g; mobile phase: 10-55% MeCN/water; to afford 2,4-difluoro-3-([[3-(trifluoromethyl)-1-[[2-(trimethylsilyl)ethoxy]methyl]pyrazolo[3,4-b]pyridin-5-yl]oxy]methyl)aniline (480 mg, 61% yield) as a white solid.

LCMS (ES, m/z): [M+H]⁺: 475

### Synthesis of 44-g: N-[2,4-difluoro-3-([[3-(trifluoromethyl)-1-[[2-(trimethylsilyl)ethoxy]methyl]pyrazolo[3,4-b]pyridin-5-yl]oxy]methyl)phenyl]-5-fluoro-2-methoxypyridine-3-sulfonamide

To a stirred solution of 2,4-difluoro-3-([[3-(trifluoromethyl)-1-[[2-(trimethylsilyl)ethoxy]methyl]pyrazolo[3,4-b]pyridin-5-yl]oxy]methyl)aniline (260 mg, 0.55 mmol, 1 equiv) in pyridine (5 mL) was added 5-fluoro-2-methoxypyridine-3-sulfonyl chloride (185 mg, 0.82 mmol, 1.5 equiv) in portions. The resulting solution was stirred for 1 h at room temperature, then quenched with water (20 mL). This mixture was extracted with EA (3×10 mL), and the combined organics were washed with brine (2×5 mL), dried over anhydrous Na₂SO₄ and concentrated. The residue was purified by Flash-Prep-HPLC with the following conditions: Column, WelFlash TM C18-I, Spherical C18 20-40 μm, 120 g; mobile phase: 20-65% MeCN/water; to afford N-[2,4-difluoro-3-([[3-(trifluoromethyl)-1-[[2-(trimethylsilyl)ethoxy]methyl]pyrazolo[3,4-b]pyridin-5-yl]oxy]methyl)phenyl]-5-fluoro-2-methoxypyridine-3-sulfonamide (250 mg, 68% yield) as a white solid.

### Synthesis of Compound 44: N-[2,4-difluoro-3-([[3-(trifluoromethyl)-1H-pyrazolo[3,4-b]pyridin-5-yl]oxy]methyl)phenyl]-5-fluoro-2-methoxypyridine-3-sulfonamide

A solution of N-[2,4-difluoro-3-([[3-(trifluoromethyl)-1-[[2-(trimethylsilyl)ethoxy]methyl]pyrazolo[3,4-b]pyridin-5-yl]oxy]methyl)phenyl]-5-fluoro-2-methoxypyridine-3-sulfonamide (200 mg, 0.3 mmol, 1 equiv) in TFA (2 mL) and DCM (2 mL) was stirred for 1 h at room temperature. The resulting mixture was concentrated under reduced pressure. The residue was purified by prep-HPLC with the following conditions: Column, welch Vltimate XB—C18, 50×250 mm, 10 μm; Mobile Phase: 30-75% MeCN/0.1% aqueous TFA; to afford N-[2,4-difluoro-3-([[3-(trifluoromethyl)-1H-pyrazolo[3,4-b]pyridin-5-yl]oxy]methyl)phenyl]-5-fluoro-2-methoxypyridine-3-sulfonamide (140 mg, 87% yield) as an off-white solid.

LCMS (ES, m/z): [M+H]⁺: 534
¹H NMR (300 MHz, DMSO-d₆) δ 8.47-8.34 (m, 2H), 7.96 (dd, J=7.3, 3.0 Hz, 1H), 7.88 (d, J=2.7 Hz, 1H), 7.38 (q, J=8.2, 7.5 Hz, 1H), 7.14 (t, J=8.9 Hz, 1H), 5.23 (s, 2H), 3.88 (s, 3H).

### Example 56: Synthesis of N-[2,4-difluoro-3-([3-methyl-1H-pyrazolo[3,4-b]pyridin-5-yl]methoxy)phenyl]-5-fluoro-2-methoxypyridine-3-sulfonamide (Compound 45)

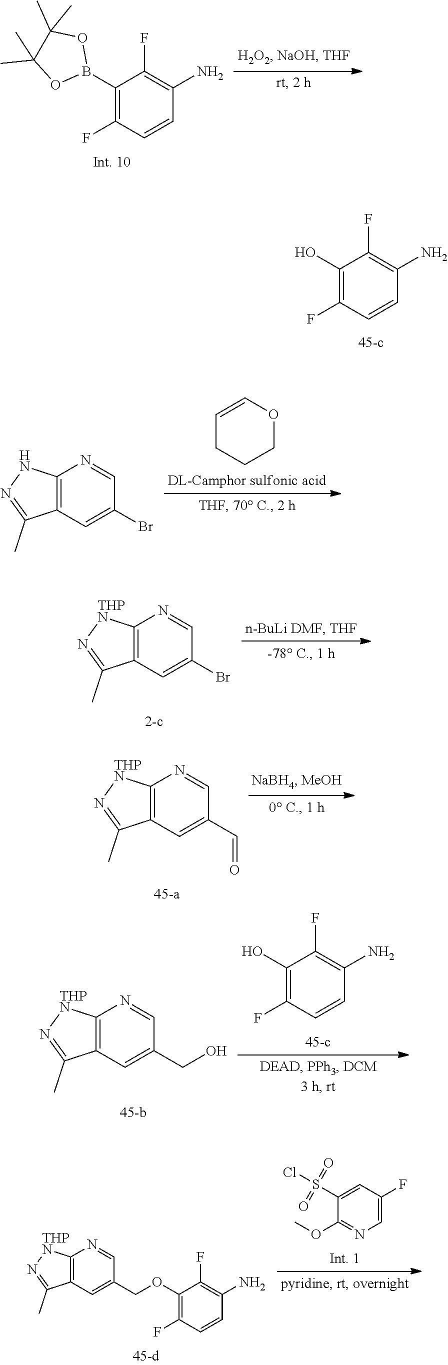

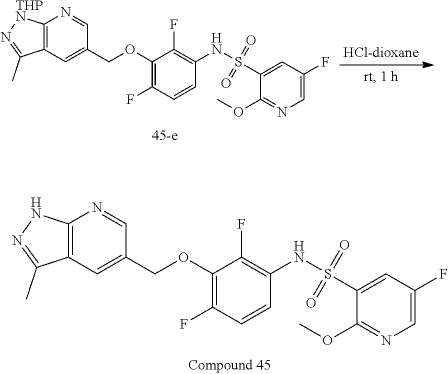

### Synthesis of 45-a: 3-methyl-1-(oxan-2-yl)pyrazolo[3,4-b]pyridine-5-carbaldehyde

Into a 500 mL 3-necked round-bottom flask was placed 5-bromo-3-methyl-1-(oxan-2-yl)pyrazolo[3,4-b]pyridine (15 g, 51 mmol, 1 equiv) and tetrahydrofuran (160 mL). This was followed by the addition of 2.5 M n-BuLi in hexane (24.4 mL, 61 mmol, 1.2 equiv) dropwise with stirring at −78° C. The solution was stirred for 1 h at −78° C., when dimethylformamide (7.4 g, 101 mmol, 2 equiv) was added dropwise at −78° C. The resulting solution was stirred for 1 h at −78° C., then quenched by the addition of 200 mL of saturated NH₄Cl/ice. This mixture was extracted with 2×200 mL of ethyl acetate and the organics were combined, dried over anhydrous sodium sulfate and concentrated. The residue was applied to a silica gel column, eluting with ethyl acetate/PE (2/1) to give 3-methyl-1-(oxan-2-yl)pyrazolo[3,4-b]pyridine-5-carbaldehyde (11 g, 88% yield) as a yellow solid.

LCMS (ES, m/z): [M+H]⁺:246

### Synthesis of 45-b: [3-methyl-1-(oxan-2-yl)pyrazolo[3,4-b]pyridin-5-yl]methanol

Into a 50 mL 3-necked round-bottom flask was placed 3-methyl-1-(oxan-2-yl)pyrazolo[3,4-b]pyridine-5-carbaldehyde (2 g, 8 mmol, 1 equiv) in MeOH (20 mL). NaBH₄ (0.37 g, 9.78 mmol, 1.2 equiv) was added in portions at 0° C. The resulting solution was stirred for 1 hr in a water/ice bath. The reaction was quenched by the addition of 50 mL of water/ice, then extracted with 3×50 mL of ethyl acetate. The combined organics were dried over anhydrous sodium sulfate and concentrated. The residue was applied to a silica gel column, eluting with ethyl acetate/petroleum ether (2/1) to give [3-methyl-1-(oxan-2-yl)pyrazolo[3,4-b]pyridin-5-yl]methanol (1.6 g, 79% yield) as a yellow solid.

LCMS (ES, m/z): [M+H]⁺:248

### Synthesis 45-c: of 3-amino-2,6-difluorophenol

Into a 50 mL round-bottom flask was placed 2,4-difluoro-3-(4,4,5,5-tetramethyl-1,3,2-dioxaborolan-2-yl)aniline (1.9 g, 7 mmol, 1 equiv), THE (10 mL), NaOH (10 mL) and 30% H₂O₂ (2.5 g, 74 mmol, 10 equiv). The reaction was stirred for 2 hr then quenched by the addition of 20 mL of saturated Na₂S₂O₄. The resulting solution was extracted with 3×20 mL of ethyl acetate, and the extracts washed with 20 ml of NaCl solution, then dried over anhydrous sodium sulfate and concentrated. The residue was applied to a silica gel column, eluting with ethyl acetate/petroleum ether (1/1) to give 3-amino-2,6-difluorophenol (0.9 g, 83% yield) as a yellow solid.

LCMS (ES, m/z): [M+H]⁺: 146

### Synthesis of 45-d: 2,4-difluoro-3-[[3-methyl-1-(oxan-2-yl)pyrazolo[3,4-b]pyridin-5-yl]methoxy]aniline

Into a 50 mL round-bottom flask was placed[3-methyl-1-(oxan-2-yl)pyrazolo[3,4-b]pyridin-5-yl]methanol (1.5 g, 6 mmol, 1 equiv), DCM (30 mL), PPh₃ (2.4 g, 9 mmol, 1.5 equiv), DEAD (1.6 g, 9 mmol, 1.5 equiv) and 3-amino-2,6-difluorophenol (0.88 g, 6 mmol, 1 equiv). The resulting solution was stirred for 3 hr then concentrated. The residue was applied to a silica gel column, eluting with ethyl acetate/petroleum ether (2/1) to give 2,4-difluoro-3-[[3-methyl-1-(oxan-2-yl)pyrazolo[3,4-b]pyridin-5-yl]methoxy]aniline (1.6 g, 70% yield) as a yellow solid.

LCMS (ES, m/z): [M+H]⁺:375

### Synthesis of 45-e: N-(2,4-difluoro-3-[[3-methyl-1-(oxan-2-yl)pyrazolo[3,4-b]pyridin-5-yl]methoxyphenyl)-5-fluoro-2-methoxypyridine-3-sulfonamide

Into an 8 mL vial was placed 2,4-difluoro-3-[[3-methyl-1-(oxan-2-yl)pyrazolo[3,4-b]pyridin-5-yl]methoxy]aniline (200 mg, 0.5 mmol, 1 equiv), pyridine (3 mL) and 5-fluoro-2-methoxypyridine-3-sulfonyl chloride (181 mg, 0.8 mmol, 1.5 equiv). The resulting solution was stirred overnight then was concentrated. The crude product was purified by Flash-Prep-HPLC with the following conditions: Column, WelFlash TM C18-I, Spherical C18 20-40 μm, 120 g; mobile phase: 5-60% MeCN/0.1% aqueous formic acid; Detector 220 nm; to give N-(2,4-difluoro-3-[[3-methyl-1-(oxan-2-yl)pyrazolo[3,4-b]pyridin-5-yl]methoxy]phenyl)-5-fluoro-2-methoxypyridine-3-sulfonamide (180 mg, 60% yield) as a yellow solid.

LCMS (ES, m/z): [M+H]⁺:564

### Synthesis of Compound 45: N-[2,4-difluoro-3-([3-methyl-1H-pyrazolo[3,4-b]pyridin-5-yl]methoxy)phenyl]-5-fluoro-2-methoxypyridine-3-sulfonamide

Into an 8 mL vial was placed N-(2,4-difluoro-3-[[3-methyl-1-(oxan-2-yl)pyrazolo[3,4-b]pyridin-5-yl]methoxy]phenyl)-5-fluoro-2-methoxypyridine-3-sulfonamide (170 mg, 0.3 mmol, 1 equiv) and 4 M HCl in 1,4-dioxane (2 mL). The resulting solution was stirred for 12 hr then concentrated. The crude product was purified by Prep-HPLC with the following conditions: Column, welch Vltimate XB—C18, 50×250 mm, 10 μm; mobile phase: 18-58% MeCN/0.1% aqueous formic acid; Detector, 220 nm; to give N-[2,4-difluoro-3-([3-methyl-1H-pyrazolo[3,4-b]pyridin-5-yl]methoxy)phenyl]-5-fluoro-2-methoxypyridine-3-sulfonamide (67 mg, 46% yield) as a white solid.

LCMS (ES, m/z): [M+H]⁺480
¹H NMR (300 MHz, DMSO-d₆) δ 13.28 (s, 1H), 10.28 (s, 1H), 8.42 (d, J=3.0 Hz, 2H), 8.16 (d, J=2.0 Hz, 1H), 7.91 (dd, J=7.3, 3.0 Hz, 1H), 7.13-7.01 (m, 1H), 6.94 (td, J=8.7, 5.4 Hz, 1H), 5.22 (s, 2H), 3.90 (s, 3H), 2.51 (s, 3H).

### Example 57: Synthesis of 5-chloro-N-[2,4-difluoro-3-([3-methyl-1H-Pyrazolo[3,4-b]pyridin-5-yl]methoxy)phenyl]-2-methoxypyridine-3-sulfonamide (Compound 46)

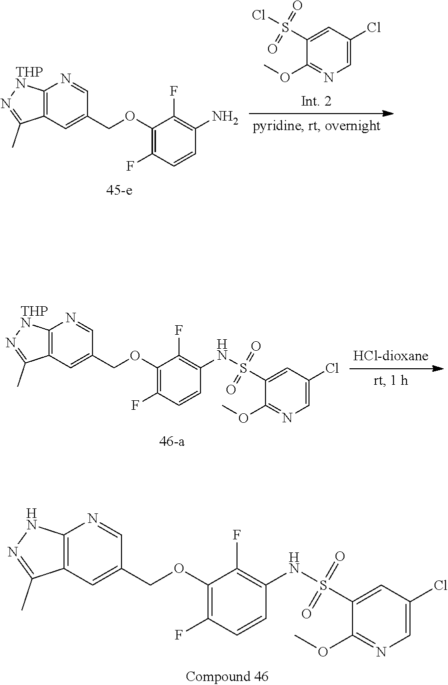

### Synthesis of 46-a: 5-chloro-N-(2,4-difluoro-3-[[3-methyl-1-(oxan-2-yl)pyrazolo[3,4-b]pyridin-5-yl]methoxyphenyl)-2-methoxypyridine-3-sulfonamide

Into an 8 mL vial was placed 2,4-difluoro-3-[[3-methyl-1-(oxan-2-yl)pyrazolo[3,4-b]pyridin-5-yl]methoxy]aniline (200 mg, 0.5 mmol, 1 equiv), pyridine (3 mL) and 5-chloro-2-methoxypyridine-3-sulfonyl chloride (194 mg, 0.8 mmol, 1.5 equiv). The resulting solution was stirred overnight then concentrated. The crude product was purified by Flash-Prep-HPLC with the following conditions: Column, WelFlash TM C18-I, Spherical C18 20-40 μm, 120 g; mobile phase: 5-60% MeCN/0.1% aqueous formic acid; Detector UV 254 nm; to give 5-chloro-N-(2,4-difluoro-3-[[3-methyl-1-(oxan-2-yl)pyrazolo[3,4-b]pyridin-5-yl]methoxy]phenyl)-2-methoxypyridine-3-sulfonamide (170 mg, 55% yield) as a yellow solid.

LCMS (ES, m/z): [M+H]⁺:580

### Synthesis of Compound 46: 5-chloro-N-[2,4-difluoro-3-([3-methyl-1H-pyrazolo[3,4-b]pyridin-5-yl]methoxy)phenyl]-2-methoxypyridine-3-sulfonamide

Into an 8 mL vial was placed 5-chloro-N-(2,4-difluoro-3-[[3-methyl-1-(oxan-2-yl)pyrazolo[3,4-b]pyridin-5-yl]methoxy]phenyl)-2-methoxypyridine-3-sulfonamide (165 mg, 0.3 mmol, 1 equiv) and 2 M HCl in 1,4-dioxane (2 mL). The resulting solution was stirred for 12 h then concentrated. The crude product was purified by Prep-HPLC with the following conditions: Column, welch Vltimate XB—C18, 50×250 mm, 10 μm; mobile phase: 25-60% MeCN/0.1% aqueous formic acid; Detector, 220 nm; to give 5-chloro-N-[2,4-difluoro-3-([3-methyl-1H-pyrazolo[3,4-b]pyridin-5-yl]methoxy)phenyl]-2-methoxypyridine-3-sulfonamide (57 mg, 47% yield) as a white solid.

LCMS (ES, m/z): [M+H]⁺: 496
¹H NMR (300 MHz, DMSO-d₆) δ 13.28 (s, 1H), 10.30 (s, 1H), 8.45 (dd, J=5.3, 2.3 Hz, 2H), 8.17 (d, J=2.0 Hz, 1H), 7.98 (d, J=2.6 Hz, 1H), 7.13-7.01 (m, 1H), 6.95 (td, J=8.7, 5.5 Hz, 1H), 5.23 (s, 2H), 3.91 (s, 3H), 2.51 (s, 3H).

### Example 58: Synthesis of N-[3-[([3-ethyl-1H-pyrazolo[3,4-b]pyridin-5-yl]oxy)methyl]-2,4-difluorophenyl]-5-fluoro-2-methylpyridine-3-sulfonamide (Compound 47)

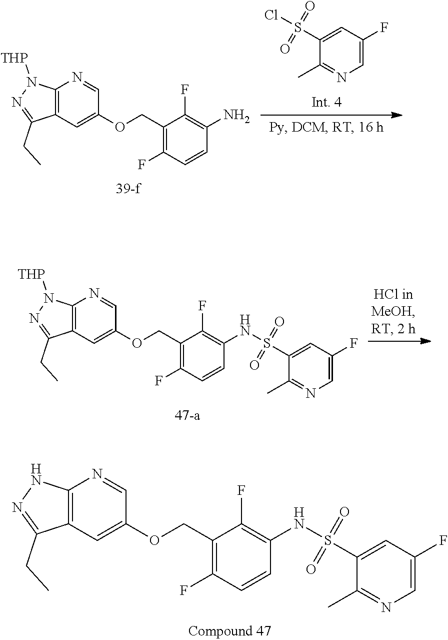

### Synthesis of 47-a: N-[3-([[3-ethyl-1-(oxan-2-yl)pyrazolo[3,4-b]pyridin-5-yl]oxy]methyl)-2,4-difluorophenyl]-5-fluoro-2-methylpyridine-3-sulfonamide

To a stirred solution of 3-([[3-ethyl-1-(oxan-2-yl)pyrazolo[3,4-b]pyridin-5-yl]oxy]methyl)-2,4-difluoroaniline (100 mg, 0.26 mmol, 1 equiv) and pyridine (163 mg, 2 mmol, 8 equiv) in DCM (3 mL) was added 5-fluoro-2-methylpyridine-3-sulfonyl chloride (162 mg, 0.77 mmol, 3 equiv) in DCM (0.5 mL) dropwise. The reaction mixture was stirred for 16 h then diluted with water (10 mL). This was extracted with DCM (3×10 mL), and the combined organics were washed with brine (20 mL), dried over anhydrous Na₂SO₄ and concentrated under reduced pressure. The residue was purified by silica gel column chromatography, eluting with PE/EtOAc (2:1) to afford N-[3-([[3-ethyl-1-(oxan-2-yl)pyrazolo[3,4-b]pyridin-5-yl]oxy]methyl)-2,4-difluorophenyl]-5-fluoro-2-methylpyridine-3-sulfonamide (110 mg, 76% yield) as a white solid.

LCMS (ES, m/z): [M+H]⁺:562.

### Synthesis of Compound 47: N-[3-[([3-ethyl-1H-pyrazolo[3,4-b]pyridin-5-yl]oxy)methyl]-2,4-difluorophenyl]-5-fluoro-2-methylpyridine-3-sulfonamide

A mixture of N-[3-([[3-ethyl-1-(oxan-2-yl)pyrazolo[3,4-b]pyridin-5-yl]oxy]methyl)-2,4-difluorophenyl]-5-fluoro-2-methylpyridine-3-sulfonamide (110 mg, 0.2 mmol, 1 equiv) in 1.25 M HCl in MeOH (2 mL) was stirred for 2 h then diluted with water (10 mL). The resulting mixture was extracted with EtOAc (3×10 mL). The combined organics were dried (MgSO₄) and concentrated under reduced pressure. The crude product was purified by Prep-HPLC with the following conditions: Column: XbridgeC18, 19*150 mm, 5 m; mobile phase: 20 mL/min, 12-28% MeCN/0.05% aqueous ammonia; to afford N-[3-[([3-ethyl-1H-pyrazolo[3,4-b]pyridin-5-yl]oxy)methyl]-2,4-difluorophenyl]-5-fluoro-2-methylpyridine-3-sulfonamide (46 mg, 45% yield) as a white solid.

LCMS (ES, m/z): [M+H]⁺: 478.
¹H NMR (300 MHz, DMSO-d₆) δ 13.09 (s, 1H), 10.71 (s, 1H), 8.70 (d, J=2.8 Hz, 1H), 8.16 (d, J=2.8 Hz, 1H), 7.93-7.82 (m, 2H), 7.37 (td, J=8.9, 5.9 Hz, 1H), 7.17 (td, J=9.0, 1.6 Hz, 1H), 5.12 (s, 2H), 2.90 (q, J=7.6 Hz, 2H), 2.75 (d, J=1.2 Hz, 3H), 1.31 (t, J=7.6 Hz, 3H).

### Example 59: Synthesis of: 5-cyano-N-(2,4-difluoro-3-((3-methyl-1H-pyrazolo[3,4-b]pyridin-5-yl)methoxy)phenyl)-2-methoxypyridine-3-sulfonamide (Compound 48)

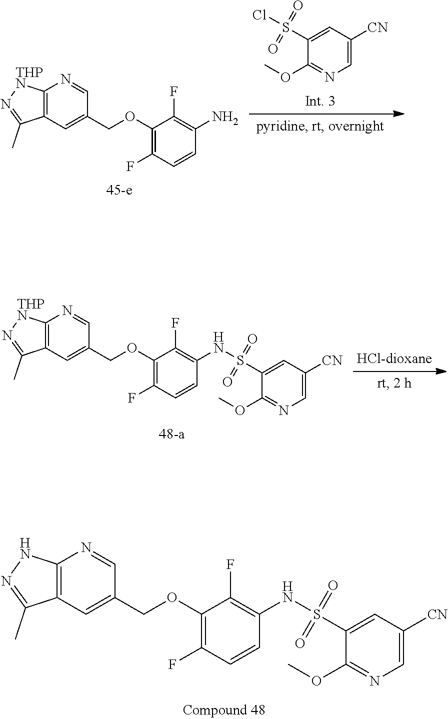

### Synthesis of 48-a: 5-cyano-N-(2,4-difluoro-3-[[3-methyl-1-(oxan-2-yl)pyrazolo[3,4-b]pyridin-5-yl]methoxy]phenyl)-2-methoxypyridine-3-sulfonamide

Into an 8 mL vial was placed 2,4-difluoro-3-[[3-methyl-1-(oxan-2-yl)pyrazolo[3,4-b]pyridin-5-yl]methoxy]aniline (200 mg, 0.5 mmol, 1 equiv), pyridine (3 mL) and 5-cyano-2-methoxypyridine-3-sulfonyl chloride (186 mg, 0.8 mmol, 1.5 equiv). The resulting solution was stirred overnight then concentrated. The crude product was purified by Flash-Prep-HPLC with the following conditions: Column, WelFlash TM C18-I, Spherical C18 20-40 μm, 120 g; mobile phase: 5-60% MeCN/0.5% aqueous formic acid; Detector 220 nm; to give 5-cyano-N-(2,4-difluoro-3-[[3-methyl-1-(oxan-2-yl)pyrazolo[3,4-b]pyridin-5-yl]methoxy]phenyl)-2-methoxypyridine-3-sulfonamide (185 mg, 61% yield) as a yellow solid.

LCMS (ES, m/z): [M+H]⁺:571

### Synthesis of Compound 48: 5-cyano-N-(2,4-difluoro-3-((3-methyl-1H-pyrazolo[3,4-b]pyridin-5-yl)methoxy)phenyl)-2-methoxypyridine-3-sulfonamide

Into a 50 mL round-bottom flask was placed 5-cyano-N-(2,4-difluoro-3-[[3-methyl-1-(oxan-2-yl)pyrazolo[3,4-b]pyridin-5-yl]methoxy]phenyl)-2-methoxypyridine-3-sulfonamide (185 mg, 0.3 mmol, 1 equiv) and 4 M HCl in 1,4-dioxane (2 mL). The resulting solution was stirred for 2 h then concentrated. The crude product was purified by Prep-HPLC with the following conditions: Column, welch Vltimate XB—C18, 50×250 mm, 10 μm; mobile phase: 15-55% MeCN/0.1% aqueous formic acid; Detector, 220 nm; to give 5-cyano-N-(2,4-difluoro-3-((3-methyl-1H-pyrazolo[3,4-b]pyridin-5-yl)methoxy)phenyl)-2-methoxypyridine-3-sulfonamide (72 mg, 46% yield) as a white solid.

LCMS (ES, m/z): [M+H]⁺: 487
¹H NMR (300 MHz, DMSO-d₆) δ 13.28 (s, 1H), 10.40 (s, 1H), 8.88 (d, J=2.3 Hz, 1H), 8.43 (dd, J=8.7, 2.1 Hz, 2H), 8.25-8.10 (m, 1H), 7.18-6.84 (m, 2H), 5.22 (s, 2H), 3.99 (s, 3H), 2.49 (d, J=1.2 Hz, 3H).

### Example 60: Synthesis of 5-cyano-N-[2,4-difluoro-3-[([3-methyl-11H-pyrazolo[3,4-b]pyridin-5-yl]methyl)amino]phenyl]-2-methoxypyridine-3-sulfonamide (Compound 49)

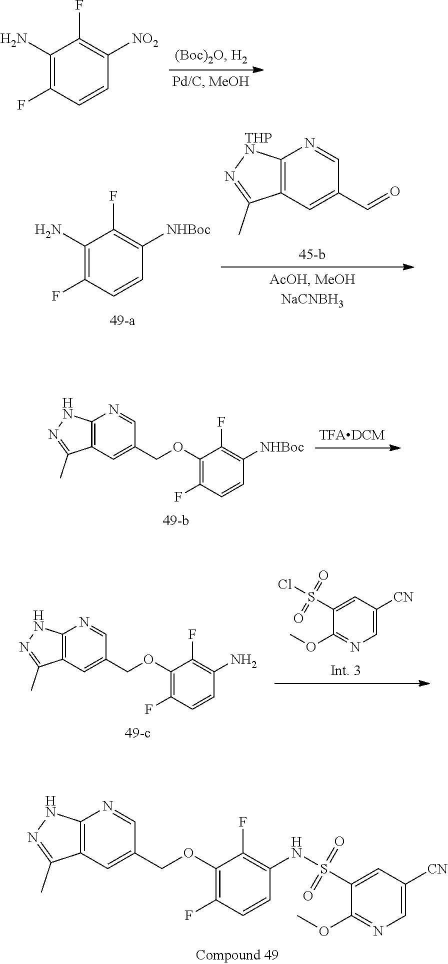

### Synthesis of 49-a: tert-butyl (3-amino-2,4-difluorophenyl)carbamate

To a stirred mixture of 2,6-difluoro-3-nitroaniline (300 mg, 1.7 mmol, 1 equiv) and Boc₂O (414 mg, 1.9 mmol, 1.1 equiv) in MeOH (10 mL) was added 10% Pd/C (30 mg, 0.28 mmol, 0.16 equiv). The reaction mixture was stirred for 5 h at room temperature under a hydrogen atmosphere. The resulting mixture was filtered; the filter cake was washed with MeOH (3×3 mL) and the filtrate concentrated. The residue was purified by silica gel column chromatography, eluting with PE/EA (3/1) to afford tert-butyl N-(3-amino-2,4-difluorophenyl)carbamate (212 mg, 50% yield) as a yellow solid.

LCMS (ES, m/z): [M+H]⁺: 245

### Synthesis of 49-b: tert-butyl N-[2,4-difluoro-3-([[3-methyl-1-(oxan-2-yl)pyrazolo[3,4-b]pyridin-5-yl]methyl]amino)phenyl]carbamate

Into a 40 mL vial were added tert-butyl N-(3-amino-2,4-difluorophenyl)carbamate (500 mg, 2 mmol, 1 equiv), 3-methyl-1-(oxan-2-yl)pyrazolo[3,4-b]pyridine-5-carbaldehyde (502 mg, 2 mmol, 1 equiv), MeOH (10 mL), HOAc (2 mL) and NaBH₃CN (386 mg, 6.1 mmol, 3 equiv). The reaction mixture was stirred overnight at 60° C., then concentrated under reduced pressure. The residue was purified by Flash-Prep-HPLC with the following conditions: Column, WelFlash TM C18-I, Spherical C18 20-40 μm, 120 g; mobile phase: 10-85% MeCN/0.1% aqueous formic acid; Detector, 220 nm; to give tert-butyl N-[2,4-difluoro-3-([[3-methyl-1-(oxan-2-yl)pyrazolo[3,4-b]pyridin-5-yl]methyl]amino)phenyl]carbamate (100 mg, 10% yield) as a white solid.

LCMS (ES, m/z): [M+H]⁺: 474

### Synthesis of 49-c: 2,6-difluoro-N1-([3-methyl-1H-pyrazolo[3,4-b]pyridin-5-yl]methyl)benzene-1,3-diamine

Into a 40 mL vial was placed tert-butyl N-[2,4-difluoro-3-([[3-methyl-1-(oxan-2-yl)pyrazolo[3,4-b]pyridin-5-yl]methyl]amino)phenyl]carbamate (100 mg, 0.2 mmol, 1 equiv), DCM (3 mL) and TFA (1 mL). The resulting solution was stirred for 3 h then concentrated. The pH was adjusted to 8 with ammonia solution and this was purified by Flash-Prep-HPLC with the following conditions: Column, WelFlash TM C18-I, Spherical C18 20-40 μm, 120 g; mobile phase: 5-55% MeCN/0.1% aqueous formic acid; Detector, 220 nm; to give 2,6-difluoro-N1-([3-methyl-1H-pyrazolo[3,4-b]pyridin-5-yl]methyl)benzene-1,3-diamine (40 mg, 65% yield) as an off-white solid.

LCMS (ES, m/z): [M+H]⁺: 290

### Synthesis of Compound 49: 5-cyano-N-[2,4-difluoro-3-[([3-methyl-1H-pyrazolo[3,4-b]pyridin-5-yl]methyl)amino]phenyl]-2-methoxypyridine-3-sulfonamide

Into an 8 mL vial were added 2,6-difluoro-N1-([3-methyl-1H-pyrazolo[3,4-b]pyridin-5-yl]methyl)benzene-1,3-diamine (40 mg, 0.14 mmol, 1 equiv) and DCM (2 mL), followed by pyridine (110 mg, 1.4 mmol, 10 equiv) and 5-cyano-2-methoxypyridine-3-sulfonyl chloride (48 mg, 0.2 mmol, 1.5 equiv). The mixture was stirred for 1 h, then concentrated. The crude product was purified by Prep-HPLC with the following conditions: Column, welch Vltimate XB—C18, 50×250 mm, 10 μm; Mobile Phase: 5-50% MeCN/0.1% aqueous formic acid; detector 220 nm; to give 5-cyano-N-[2,4-difluoro-3-[([3-methyl-1H-pyrazolo[3,4-b]pyridin-5-yl]methyl)amino]phenyl]-2-methoxypyridine-3-sulfonamide (9 mg, 13% yield) as a white solid.

LCMS (ES, m/z): [M+H]⁺: 486
¹H NMR (300 MHz, DMSO-d₆) δ 13.11 (s, 1H), 10.14 (s, 1H), 8.80 (d, J=2.2 Hz, 1H), 8.36 (d, J=2.1 Hz, 2H), 7.97 (d, J=2.0 Hz, 1H), 6.82 (t, J=10.3 Hz, 1H), 6.51-6.40 (m, 1H), 6.00-5.91 (m, 1H), 4.42 (s, 2H), 3.95 (s, 3H), 2.46 (s, 3H).

### Example 61: Synthesis of 5-cyano-N-[2,4-difluoro-3-[([3-methyl-1H-pyrazolo[3,4-b]pyridin-5-yl]oxy)methyl]phenyl]-2-methoxypyridine-3-sulfonamide (Compound 50)

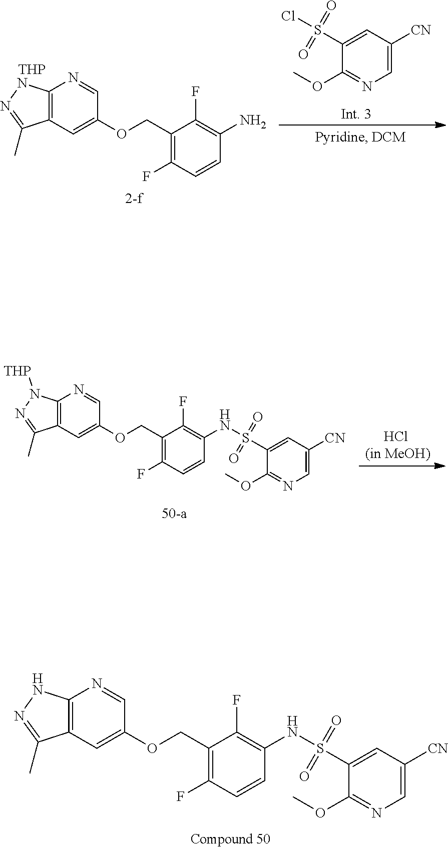

### Synthesis of 50-a: N-[2,4-difluoro-3-([[3-methyl-1-(oxan-2-yl)pyrazolo[3,4-b]pyridin-5-yl]oxy]methyl)phenyl]-5-fluoro-2-methoxypyridine-3-sulfonamide

To a stirred solution of 2,4-difluoro-3-([[3-methyl-1-(oxan-2-yl)pyrazolo[3,4-b]pyridin-5-yl]oxy]methyl) aniline (100 mg, 0.27 mmol, 1 equiv) and pyridine (84 mg, 1.1 mmol, 4 equiv) in DCM (2 mL) was added 5-cyano-2-methoxypyridine-3-sulfonyl chloride (93 mg, 0.4 mmol, 1.5 equiv) in portions at room temperature. The resulting mixture was stirred for 4 h then diluted with water (2 mL). This was extracted with DCM (3×10 mL), and the combined organics were washed with water (2×10 mL) and brine (10 mL), dried over anhydrous Na₂SO₄ and concentrated. The residue was purified by silica gel column chromatography, eluted with EtOAc/PE (0-50%) to afford N-[2,4-difluoro-3-([[3-methyl-1-(oxan-2-yl)pyrazolo[3,4-b]pyridin-5-yl]oxy]methyl)phenyl]-5-fluoro-2-methoxypyridine-3-sulfonamide (120 mg, 80% yield) as a yellow solid.

LCMS (ES, m/z): [M+H]⁺: 571.

### Synthesis of Compound 50: 5-cyano-N-[2,4-difluoro-3-[([3-methyl-1H-pyrazolo[3,4-b]pyridin-5-yl]oxy)methyl]phenyl]-2-methoxypyridine-3-sulfonamide

To a stirred mixture of N-[2,4-difluoro-3-([[3-methyl-1-(oxan-2-yl)pyrazolo[3,4-b]pyridin-5-yl]oxy]methyl)phenyl]-5-fluoro-2-methoxypyridine-3-sulfonamide (100 mg, 0.17 mmol, 1 equiv) was added 4 M HCl in MeOH (1 mL, 4 mmol, 22 equiv) dropwise at 0-5° C. The resulting mixture was stirred for 1 h at room temperature, then concentrated. The residue was neutralized to pH 7 with 5% ammonia solution and purified by Prep-HPLC with the following conditions: Column: welch Vltimate XB—C18, 50×250 mm, 10 m; mobile phase: 90 mL/min, 20-50% MeCN/0.1% aqueous formic acid; to afford 5-cyano-N-[2,4-difluoro-3-[([3-methyl-1H-pyrazolo[3,4-b]pyridin-5-yl]oxy)methyl]phenyl]-2-methoxypyridine-3-sulfonamide (34 mg, 40% yield) as a white solid.

LCMS (ES, m/z): [M+H]⁺: 487.
¹H NMR (300 MHz, DMSO-d₆) δ 13.09 (s, 1H), 8.89 (d, J=2.3 Hz, 1H), 8.45 (d, J=2.2 Hz, 1H), 8.19 (d, J=2.7 Hz, 1H), 7.86 (d, J=2.8 Hz, 1H), 7.38 (td, J=9.0, 5.9 Hz, 1H), 7.14 (t, J=8.9 Hz, 1H), 5.13 (s, 2H), 4.00 (s, 3H), 2.48 (s, 3H).

### Example 62: Synthesis of N-[2,4-difluoro-3-([3-methyl-1H-pyrazolo[3,4-b]pyridin-5-yl]methoxy)phenyl]-5-fluoro-2-methylpyridine-3-sulfonamide (Compound 51)

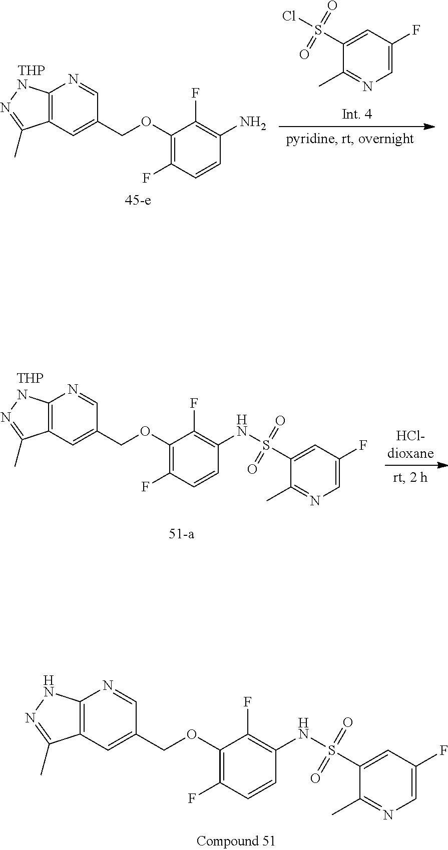 [SMILES: Cc1n[nH]c2ncc(COc3c(F)ccc(N)c3F)cc12 ; Cc1ncc(F)cc1S(=O)(=O)Cl ; Cc1ncc(F)cc1S(=O)(=O)Nc1ccc(F)c(OCc2cnc3[nH]nc(C)c3c2)c1F ; Cc1ncc(F)cc1S(=O)(=O)Nc1ccc(F)c(OCc2cnc3[nH]nc(C)c3c2)c1F ; "THP" ; "THP"]

### Synthesis of 51-a: N-(2,4-difluoro-3-[[3-methyl-oxan-2-yl)pyrazolo[3,4-b]pyridin-5-yl]methoxy]phenyl)-5-fluoro-2-methylpyridine-3-sulfonamide

Into an 8 mL vial was placed 2,4-difluoro-3-[[3-methyl-1-(oxan-2-yl)pyrazol o[3,4-b]pyri din-5-yl]methoxy]aniline (200 mg, 0.5 mmol, 1 equiv), pyridine (3 mL) and 5-fluoro-2-methylpyridine-3-sulfonyl chloride (224 mg, 1 mmol, 2 equiv) and the solution stirred overnight. The reaction mixture was concentrated, and the crude product purified by Flash-Prep-HPLC with the following conditions: Column, WelFlash TM C18-I, Spherical C18 20-40 μm, 120 g; mobile phase: 5-60% MeCN/0.5% aqueous formic acid; Detector 220 nm; to give N-(2,4-difluoro-3-[[3-methyl-1-(oxan-2-yl)pyrazolo[3,4-b]pyridin-5-yl]methoxy]phenyl)-5-fluoro-2-methylpyridine-3-sulfonamide (210 mg, 72% yield) as a yellow solid.

LCMS (ES, m/z): [M+H]⁺: 548

### Synthesis of Compound 51: N-[2,4-difluoro-3-([3-methyl-1H-pyrazolo[3,4-b]pyridin-5-yl]methoxy)phenyl]-5-fluoro-2-methylpyridine-3-sulfonamide

Into an 8 mL vial was placed N-(2,4-difluoro-3-[[3-methyl-1-(oxan-2-yl)pyrazolo[3,4-b]pyridin-5-yl]methoxy]phenyl)-5-fluoro-2-methylpyridine-3-sulfonamide (205 mg, 0.4 mmol, 1 equiv) and 4 M HCl in 1,4-dioxane (2 mL). The resulting solution was stirred for 2 h then concentrated. The residue was purified by prep-HPLC with the following conditions: Column, welch Vltimate XB—C18, 50×250 mm, 10 μm; Mobile Phase: 20 MeCN/0.1% aqueous formic acid; detector, 220 nm; to give N-[2,4-difluoro-3-([3-methyl-1H-pyrazolo[3,4-b]pyridin-5-yl]methoxy)phenyl]-5-fluoro-2-methylpyridine-3-sulfonamide (100 mg, 58% yield) as a white solid.

LCMS (ES, m/z): [M+H]⁺: 464
¹H NMR (300 MHz, DMSO-d₆) δ 13.28 (s, 1H), 10.61 (s, 1H), 8.69 (d, J=2.8 Hz, 1H), 8.43 (d, J=2.0 Hz, 1H), 8.16 (d, J=2.0 Hz, 1H), 7.83 (dd, J=8.2, 2.9 Hz, 1H), 7.07 (td, J=10.5, 9.9, 1.9 Hz, 1H), 6.93 (td, J=8.8, 5.5 Hz, 1H), 5.21 (s, 2H), 2.69 (d, J=1.2 Hz, 3H), 2.49 (d, J=1.2 Hz, 3H).

### Example 63: Synthesis of N-[2,4-difluoro-3-[([3-isopropyl-1H-pyrazolo[3,4-b]pyridin-5-yl]oxy)methyl]phenyl]-5-fluoro-2-methoxypyridine-3-sulfonamide (Compound 52)

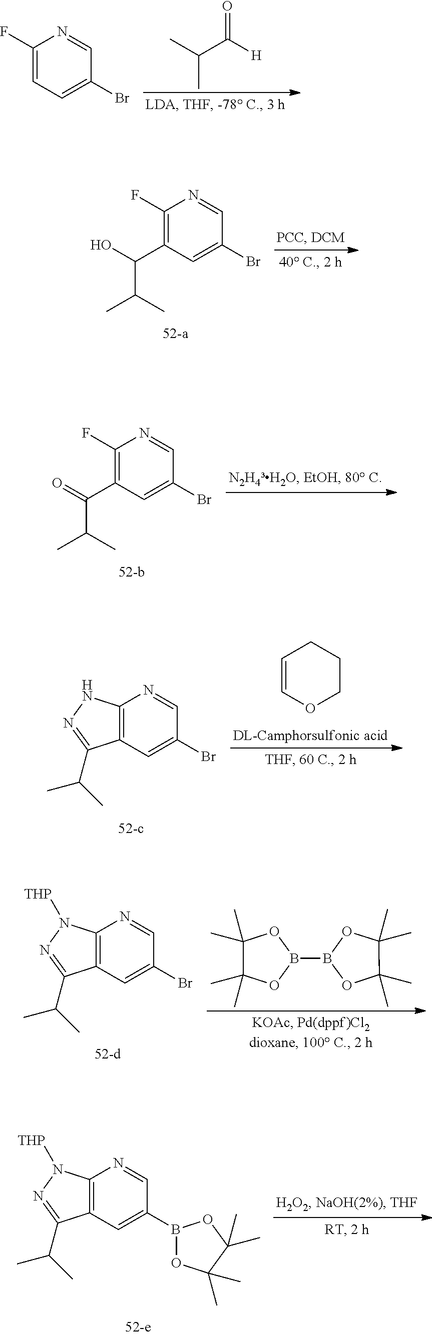

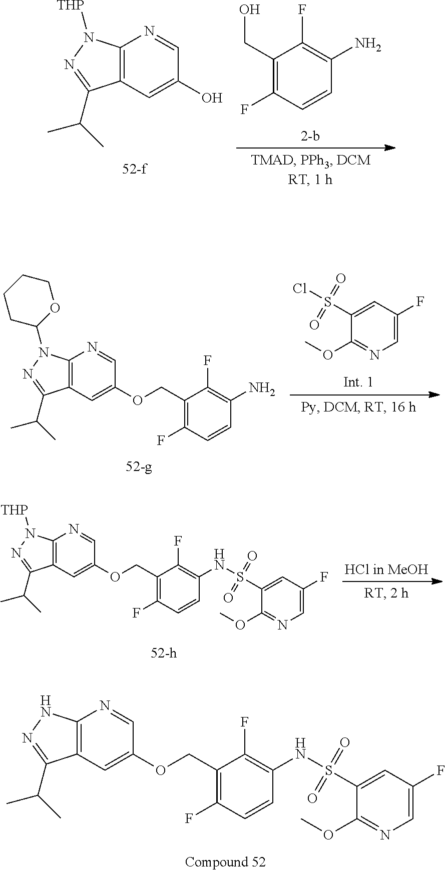

### Synthesis of 52-a: 1-(5-bromo-2-fluoropyridin-3-yl)-2-methylpropan-1-ol

To a stirred solution of 5-bromo-2-fluoropyridine (10 g, 57 mmol, 1 equiv) in THF (200 mL) was added 2M LDA in THF solution (37 mL, 74 mmol, 1.3 equiv) dropwise at −78° C. under nitrogen atmosphere. The solution was stirred for 30 min at −60° C., then isobutyraldehyde (4.9 g, 68 mmol, 1.2 equiv) was added dropwise over 5 min at −78° C. The resulting mixture was stirred for 1 h at −30° C., then quenched with sat. NH₄Cl (aq.) at low temperature. This was extracted with EtOAc (3×100 mL), and the combined organics were washed with brine (300 mL), dried over anhydrous Na₂SO₄ and concentrated under reduced pressure. The residue was purified by silica gel column chromatography, eluting with PE/EtOAc (7:1) to afford 1-(5-bromo-2-fluoropyridin-3-yl)-2-methylpropan-1-ol (8 g, 57% yield) as a light yellow oil.

LCMS (ES, m/z): [M+H]⁺: 248.

### Synthesis of 52-b: 1-(5-bromo-2-fluoropyridin-3-yl)-2-methylpropan-1-one

To a stirred solution of 1-(5-bromo-2-fluoropyridin-3-yl)-2-methylpropan-1-ol (8 g, 32 mmol, 1 equiv) in DCM (150 mL) was added pyridinium chlorochromate (13.9 g, 65 mmol, 2 equiv). The resulting mixture was stirred for 3 h at 40° C., then filtered, the filter cake being washed with DCM (3×20 mL). The filtrate was concentrated and the residue was purified by silica gel column chromatography, eluting with PE/EtOAc (5:1) to afford 1-(5-bromo-2-fluoropyridin-3-yl)-2-methylpropan-1-one (5 g, 63% yield) as a light yellow solid.

LCMS (ES, m/z): [M+H]⁺: 246.

### Synthesis of 52-c: 5-bromo-3-isopropyl-1H-pyrazolo[3,4-b]pyridine

To a stirred solution of 1-(5-bromo-2-fluoropyridin-3-yl)-2-methylpropan-1-one (5 g, 20 mmol, 1 equiv) in EtOH (60 mL) was added hydrazine hydrate (3.05 g, 61 mmol, 3 equiv) at room temperature under nitrogen atmosphere. The resulting mixture was stirred for 2 h at 80° C., then concentrated. The residue was purified by silica gel column chromatography, eluting with PE/EtOAc (2:1) to afford 5-bromo-3-isopropyl-1H-pyrazolo[3,4-b]pyridine (2.5 g, 51% yield) as a light yellow solid.

LCMS (ES, m/z): [M+H]⁺: 240.

### Synthesis of 52-d: 5-bromo-3-isopropyl-1-(oxan-2-yl)pyrazolo[3,4-b]pyridine

To a stirred solution of 5-bromo-3-isopropyl-1H-pyrazolo[3,4-b]pyridine (2.4 g, 10 mmol, 1 equiv) and dihydropyran (4.2 g, 50 mmol, 5 equiv) in THE (40 mL) was added DL-Camphorsulfonic acid (347 mg, 1.5 mmol, 0.15 equiv). The reaction was stirred for 2 h at 60° C., then concentrated under reduced pressure. The residue was purified by silica gel column chromatography, eluting with PE/EtOAc (5:1) to afford 5-bromo-3-isopropyl-1-(oxan-2-yl)pyrazolo[3,4-b]pyridine (2.5 g, 77% yield) as a light yellow oil.

LCMS (ES, m/z): [M+H]⁺: 324.

### Synthesis of 52-e: 3-isopropyl-1-(tetrahydro-2H-pyran-2-yl)-5-(4,4,5,5-tetramethyl-1,3,2-dioxaborolan-2-yl)-1H-pyrazolo[3,4-b]pyridine

To a solution of 5-bromo-3-isopropyl-1-(oxan-2-yl)pyrazolo[3,4-b]pyridine (2.6 g, 8 mmol, 1 equiv) and bis(pinacolato)diboron (3.05 g, 12 mmol, 1.5 equiv) in dioxane (50 mL) were added KOAc (1.57 g, 16 mmol, 2 equiv) and Pd(dppf)Cl₂ (587 mg, 0.8 mmol, 0.1 equiv). After stirring for 2 h at 100° C. under nitrogen atmosphere, desired product could be detected by LCMS. The resulting mixture was used in the next step directly without any workup.

LCMS (ES, m/z): [M+H]⁺: 372.

### Synthesis of 52-f: 3-isopropyl-1-(oxan-2-yl)pyrazolo[3,4-b]pyridin-5-ol

To the solution from the previous step was added THE (50 mL) and NaOH (2% aq., 40 mL), followed by 30% H₂O₂ (7.6 g, 67 mmol, 8 equiv) dropwise at room temperature. The mixture was stirred for 2 h at room temperature under air, then diluted with water (50 mL). This was extracted with EtOAc (3×30 mL), and the combined organics were washed with brine (50 mL), dried over anhydrous Na₂SO₄ and concentrated. The residue was purified by silica gel column chromatography, eluting with PE/EtOAc (1:1) to afford 3-isopropyl-1-(oxan-2-yl)pyrazolo[3,4-b]pyridin-5-ol (1.5 g, 85% yield) as a yellow oil.

LCMS (ES, m/z): [M+H]⁺:262.

### Synthesis of 52-g: 2,4-difluoro-3-([[3-isopropyl-1-(oxan-2-yl)pyrazolo[3,4-b]pyridin-5-yl]oxy]methyl)aniline

To a stirred solution of 3-isopropyl-1-(oxan-2-yl)pyrazolo[3,4-b]pyridin-5-ol (750 mg, 2.8 mmol, 1 equiv), (3-amino-2,6-difluorophenyl)methanol (548 mg, 3.4 mmol, 1.2 equiv) and PPh₃ (1.13 g, 4.3 mmol, 1.5 equiv) in DCM (15 mL) was added TMAD (741 mg, 4.3 mmol, 1.5 equiv) at room temperature. The reaction mixture was stirred for 1 h, then diluted with water (30 mL). The resulting mixture was extracted with DCM (2×30 mL), and the combined organics were washed with brine (20 mL), dried over anhydrous Na₂SO₄ and concentrated under reduced pressure. The residue was purified by silica gel column chromatography, eluting with PE/EtOAc (3:1) to afford 2,4-difluoro-3-([[3-isopropyl-1-(oxan-2-yl)pyrazolo[3,4-b]pyridin-5-yl]oxy]methyl)aniline (600 mg, 52% yield) as off-white solid.

LCMS (ES, m/z): [M+H]⁺: 403.

### Synthesis of 52-h: N-[2,4-difluoro-3-([[3-isopropyl-1-(oxan-2-yl)pyrazolo[3,4-b]pyridin-5-yl]oxyl methyl)phenyl-5-fluoro-2-methoxypyridine-3-sulfonamide

To a stirred solution of 2,4-difluoro-3-([[3-isopropyl-1-(oxan-2-yl)pyrazolo[3,4-b]pyridin-5-yl]oxy]methyl)aniline (100 mg, 0.25 mmol, 1 equiv) and pyridine (78 mg, 1 mmol, 4 equiv) in DCM (3 mL) was added 5-fluoro-2-methoxypyridine-3-sulfonyl chloride (73 mg, 0.32 mmol, 1.3 equiv) in DCM (0.5 mL) dropwise. The resulting mixture was stirred for 16 h then concentrated. The residue was purified by silica gel column chromatography, eluting with PE/EtOAc (4:1) to afford N-[2,4-difluoro-3-([[3-isopropyl-1-(oxan-2-yl)pyrazolo[3,4-b]pyridin-5-yl]oxy]methyl)phenyl]-5-fluoro-2-methoxypyridine-3-sulfonamide (110 mg, 75% yield) as a light yellow solid.

LCMS (ES, m/z): [M+H]⁺: 592.

### Synthesis of Compound 52: N-[2,4-difluoro-3-[([3-isopropyl-1H-pyrazolo[3,4-b]pyridin-5-yl]oxy)methyl]phenyl]-5-fluoro-2-methoxypyridine-3-sulfonamide

A solution of N-[2,4-difluoro-3-([[3-isopropyl-1-(oxan-2-yl)pyrazolo[3,4-b]pyridin-5-yl]oxy]methyl)phenyl]-5-fluoro-2-methoxypyridine-3-sulfonamide (110 mg, 0.2 mmol, 1 equiv) in 4 M HCl in MeOH (2 mL) was stirred for 2 h then concentrated. The residue was purified by Prep-HPLC with the following conditions: Column: XbridgeC18, 19*150 mm, 5 μm; mobile phase: 20 mL/min, 16-38% MeCN/0.05% aqueous ammonia; to afford N-[2,4-difluoro-3-[([3-isopropyl-1H-pyrazolo[3,4-b]pyridin-5-yl]oxy)methyl]phenyl]-5-fluoro-2-methoxypyridine-3-sulfonamide (50 mg, 49% yield) as a white solid.

LCMS (ES, m/z): [M+H]⁺: 508.
¹H NMR (300 MHz, DMSO-d₆) δ 13.06 (s, 1H), 10.35 (s, 1H), 8.43 (d, J=3.0 Hz, 1H), 8.16 (s, 1H), 8.02-7.93 (m, 1H), 7.88 (s, 1H), 7.37 (d, J=7.8 Hz, 1H), 7.15 (t, J=8.9 Hz, 1H), 5.16 (s, 2H), 3.90 (s, 3H), 3.30-3.28 (m, 1H), 1.36 (d, J=6.9 Hz, 6H).

### Example 64: Synthesis of 5-chloro-N-[2,4-difluoro-3-[([3-isopropyl-1H-pyrazolo[3,4-b]pyridin-5-yl]oxy)methyl]phenyl]-2-methoxypyridine-3-sulfonamide (Compound 53)

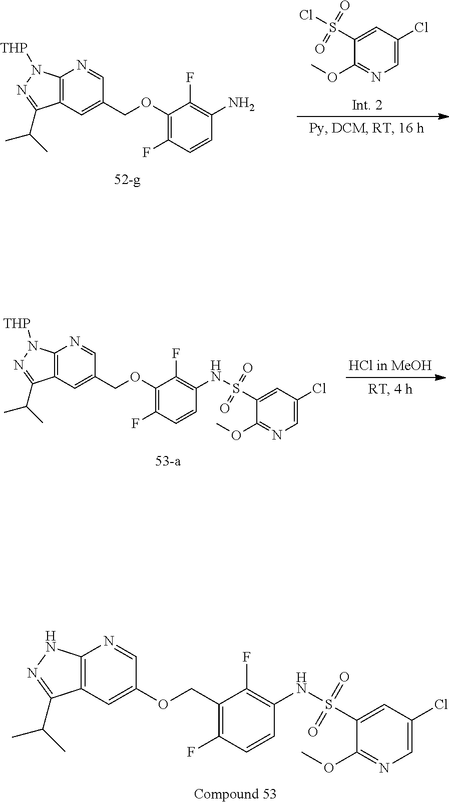

### Synthesis of 53-a: 5-chloro-N-[2,4-difluoro-3-([[3-isopropyl-1-(oxan-2-yl)pyrazolo[3,4-b]pyridin-5-yl]oxyl methyl)phenyl]-2-methoxypyridine-3-sulfonamide

To a stirred solution of 2,4-difluoro-3-([[3-isopropyl-1-(oxan-2-yl)pyrazolo[3,4-b]pyridin-5-yl]oxy]methyl)aniline (100 mg, 0.25 mmol, 1 equiv) and pyridine (78 mg, 0.1 mmol, 4 equiv) in DCM (3 mL) was added 5-chloro-2-methoxypyridine-3-sulfonyl chloride (78 mg, 0.32 mmol, 1.3 equiv) in DCM (0.5 mL) dropwise, and the reaction was stirred for 16 h, before being concentrated. The residue was purified by silica gel column chromatography, eluting with PE/EtOAc (5:1) to afford 5-chloro-N-[2,4-difluoro-3-([[3-isopropyl-1-(oxan-2-yl)pyrazolo[3,4-b]pyridin-5-yl]oxy]methyl)phenyl]-2-methoxypyridine-3-sulfonamide (120 mg, 79% yield) as a light yellow solid.

LCMS (ES, m/z): [M+H]⁺:608.

### Synthesis of Compound 53: 5-chloro-N-[2,4-difluoro-3-[([3-isopropyl-1H-pyrazolo[3,4-b]pyridin-5-yl]oxy)methyl]phenyl]-2-methoxypyridine-3-sulfonamide

A solution of 5-chloro-N-[2,4-difluoro-3-([[3-isopropyl-1-(oxan-2-yl)pyrazolo[3,4-b]pyridin-5-yl]oxy]methyl)phenyl]-2-methoxypyridine-3-sulfonamide (120 mg, 0.2 mmol, 1 equiv) in 4N HCl in MeOH (2 mL) was stirred for 2 h then diluted with water (10 mL). The aqueous layer was extracted with EtOAc (2×10 mL), and the combined organics were washed with brine (10 mL), dried over anhydrous Na₂SO₄ and concentrated under reduced pressure. The crude product was purified by Prep-HPLC with the following conditions: Column: XbridgeC18, 19*150 mm, 5 m; mobile phase: 20 mL/min, 17-42% MeCN/0.05% aqueous ammonia; to afford 5-chloro-N-[2,4-difluoro-3-[([3-isopropyl-1H-pyrazolo[3,4-b]pyridin-5-yl]oxy)methyl]phenyl]-2-methoxypyridine-3-sulfonamide (50 mg, 45% yield) as a white solid.

LCMS (ES, m/z): [M+H]⁺: 524.
¹H NMR (300 MHz, DMSO-d₆) δ13.05 (s, 1H), 10.37 (s, 1H), 8.47 (d, J=2.5 Hz, 1H), 8.17 (s, 1H), 8.03 (d, J=2.5 Hz, 1H), 7.88 (s, 1H), 7.37 (d, J=8.3 Hz, 1H), 7.16 (t, J=9.1 Hz, 1H), 5.16 (s, 2H), 3.91 (s, 3H), 3.30-3.28 (m, 1H), 1.37 (d, J=7.0 Hz, 6H).

### Example 65: Synthesis of N-[2,4-difluoro-3-[([3-isopropyl-1H-pyrazolo[3,4-b]pyridin-5-yl]oxy)methyl]phenyl]-5-fluoro-2-methylpyridine-3-sulfonamide (Compound 54)

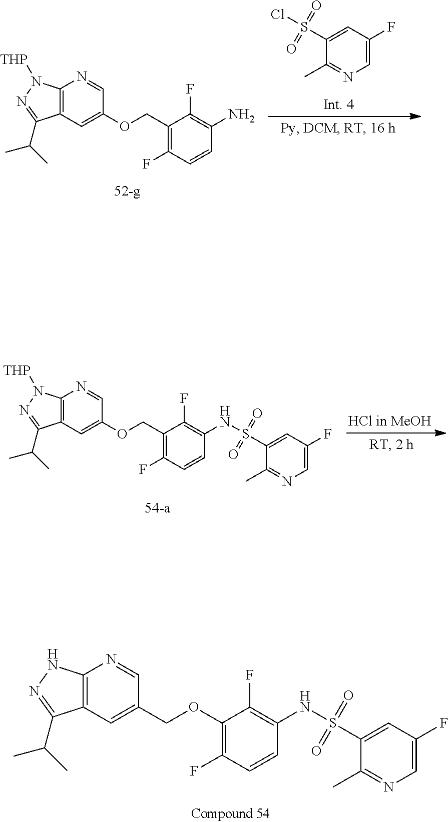

### Synthesis of 54-a: N-[2,4-difluoro-3-([[3-isopropyl-1-(oxan-2-yl)pyrazolo[3,4-b]pyridin-5-yl]oxy]methyl)phenyl]-5-fluoro-2-methylpyridine-3-sulfonamide

To a stirred solution of 2,4-difluoro-3-([[3-isopropyl-1-(oxan-2-yl)pyrazolo[3,4-b]pyridin-5-yl]oxy]methyl)aniline (100 mg, 0.25 mmol, 1 equiv) and DIEA (129 mg, 1 mmol, 4 equiv) in DCM (3 mL) was added 5-fluoro-2-methylpyridine-3-sulfonyl chloride (156 mg, 0.75 mmol, 3 equiv) in DCM (0.5 mL) dropwise. The reaction mixture was stirred for 20 h then diluted with water (10 mL). This was extracted with DCM (2×10 mL), and the combined organics washed with brine (2×10 mL), dried over anhydrous Na₂SO₄ and concentrated under reduced pressure. The residue was purified by silica gel column chromatography, eluting with PE/EtOAc (4:1) to afford N-[2,4-difluoro-3-([[3-isopropyl-1-(oxan-2-yl)pyrazolo[3,4-b]pyridin-5-yl]oxy]methyl)phenyl]-5-fluoro-2-methylpyridine-3-sulfonamide (100 mg, 70% yield) as a yellow solid.

LCMS (ES, m/z): [M+H]⁺: 576.

### Synthesis of Compound 54: N-[2,4-difluoro-3-[([3-isopropyl-1H-pyrazolo[3,4-b]pyridin-5-yl]oxy)methyl]phenyl]-5-fluoro-2-methylpyridine-3-sulfonamide

A solution of N-[2,4-difluoro-3-([[3-isopropyl-1-(oxan-2-yl)pyrazolo[3,4-b]pyridin-5-yl]oxy]methyl)phenyl]-5-fluoro-2-methylpyridine-3-sulfonamide (100 mg, 0.19 mmol, 1 equiv) in 4 M HCl in MeOH (1.5 mL) was stirred for 2 h then diluted with water (10 mL). The resulting mixture was extracted with EtOAc (2×10 mL). The combined organics were washed with brine (10 mL), dried over anhydrous Na₂SO₄ and concentrated under reduced pressure. The crude product was purified by Prep-HPLC with the following conditions: Column: XbridgeC18, 19*150 mm, 5 m; mobile phase: 20 mL/min, 13-35% MeCN/0.05% aqueous ammonia; to afford N-[2,4-difluoro-3-[([3-isopropyl-1H-pyrazolo[3,4-b]pyridin-5-yl]oxy)methyl]phenyl]-5-fluoro-2-methylpyridine-3-sulfonamide (37 mg, 39% yield) as a white solid.

LCMS (ES, m/z): [M+H]⁺: 492.
¹H NMR (300 MHz, DMSO-d₆) δ13.05 (s, 1H), 10.70 (s, 1H), 8.70 (d, J=2.8 Hz, 1H), 8.15 (d, J=2.7 Hz, 1H), 7.92-7.83 (m, 2H), 7.37 (td, J=8.9, 5.9 Hz, 1H), 7.16 (td, J=9.0, 1.7 Hz, 1H), 5.14 (s, 2H), 3.30-3.26 (m, 1H), 2.78-2.72 (m, 3H), 1.36 (d, J=6.9 Hz, 6H).

### Example 66: Synthesis of 5-cyano-N-[2,4-difluoro-3-[([3-isopropyl-1H-pyrazolo[3,4-b]pyridin-5-yl]oxy)methyl]phenyl]-2-methoxypyridine-3-sulfonamide (Compound 55)

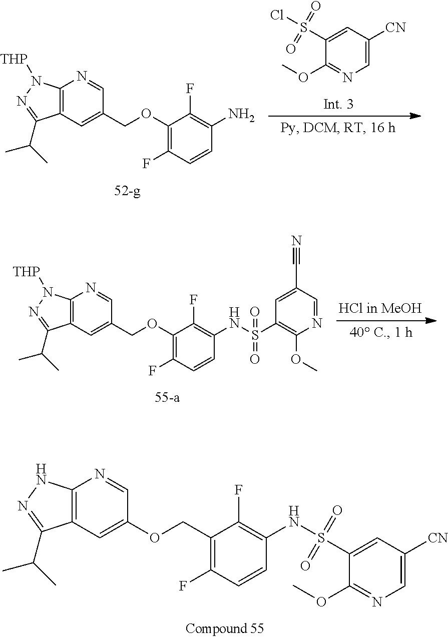

### Synthesis of 55-a: 5-cyano-N-[2,4-difluoro-3-([[3-isopropyl-1-(oxan-2-yl)pyrazolo[3,4-b]pyridin-5-yl]oxy]methyl)phenyl]-2-methoxypyridine-3-sulfonamide

To a stirred solution of 2,4-difluoro-3-([[3-isopropyl-1-(oxan-2-yl)pyrazolo[3,4-b]pyridin-5-yl]oxy]methyl)aniline (100 mg, 0.25 mmol, 1 equiv) and pyridine (79 mg, 1 mmol, 4 equiv) in DCM (3 mL) was added 5-cyano-2-methoxypyridine-3-sulfonyl chloride (75 mg, 0.32 mmol, 1.3 equiv) in DCM (0.5 mL) dropwise. The resulting mixture was stirred for 16 h then concentrated. The residue was purified by silica gel column chromatography, eluting with PE/EtOAc (4:1) to afford 5-cyano-N-[2,4-difluoro-3-([[3-isopropyl-1-(oxan-2-yl)pyrazolo[3,4-b]pyridin-5-yl]oxy]methyl)phenyl]-2-methoxypyridine-3-sulfonamide (120 mg, 81% yield) as a yellow solid.

LCMS (ES, m/z): [M+H]⁺:599.

### Synthesis of Compound 55: 5-cyano-N-[2,4-difluoro-3-[([3-isopropyl-1H-pyrazolo[3,4-b]pyridin-5-yl]oxy)methyl]phenyl]-2-methoxypyridine-3-sulfonamide

A solution of 5-cyano-N-[2,4-difluoro-3-([[3-isopropyl-1-(oxan-2-yl)pyrazolo[3,4-b]pyridin-5-yl]oxy]methyl)phenyl]-2-methoxypyridine-3-sulfonamide (120 mg, 0.2 mmol, 1 equiv) in 4 M HCl in MeOH (2 mL) was stirred for 1 h at 40° C. The resulting mixture was diluted with water (10 mL) and extracted with EtOAc (2×10 mL). The combined organics were washed with brine (10 mL), dried over anhydrous Na₂SO₄ and concentrated. The crude product was purified by Prep-HPLC with the following conditions: Column: XbridgeC18, 19*150 mm, 5 μm; mobile phase: 20 mL/min, 17-47% MeCN/0.05% aqueous ammonia; to afford 5-cyano-N-[2,4-difluoro-3-[([3-isopropyl-1H-pyrazolo[3,4-b]pyridin-5-yl]oxy)methyl]phenyl]-2-methoxypyridine-3-sulfonamide (55 mg, 53% yield) as a white solid.

LCMS (ES, m/z): [M+H]⁺: 515.
¹H NMR (300 MHz, DMSO-d₆) δ13.05 (s, 1H), 10.47 (s, 1H), 8.91 (d, J=2.3 Hz, 1H), 8.45 (d, J=2.3 Hz, 1H), 8.17 (d, J=2.7 Hz, 1H), 7.88 (d, J=2.8 Hz, 1H), 7.46-7.32 (m, 1H), 7.21-7.09 (m, 1H), 5.15 (s, 2H), 4.00 (s, 3H), 3.27, (m, 1H), 1.37 (d, J=6.9 Hz, 6H).

### Example 67: Synthesis of N-[3,5-difluoro-4-[([3-methyl-1H-pyrazolo[3,4-b]pyridin-5-yl]oxy)methyl]pyridin-2-yl]-5-fluoro-2-methoxypyridine-3-sulfonamide (Compound 56)

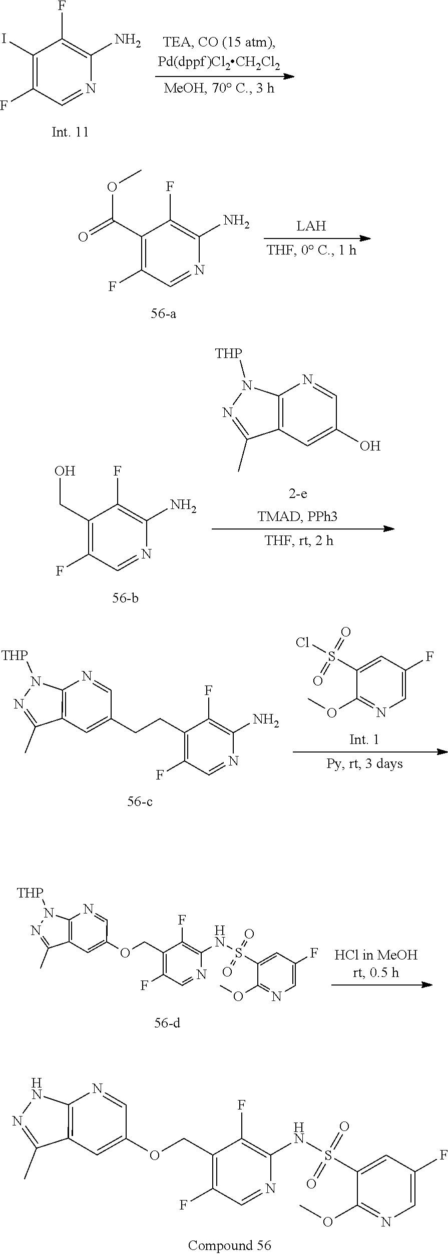

### Synthesis of 56-a: methyl 2-amino-3,5-difluoropyridine-4-carboxylate

To a solution of 3,5-difluoro-4-iodopyridin-2-amine (640 mg, 2.5 mmol, 1 equiv) and TEA (760 mg, 7.5 mmol, 3 equiv) in MeOH (30 mL) was added Pd(dppf)Cl₂·CH₂Cl₂ (204 mg, 0.25 mmol, 0.1 equiv) in a pressure tank. The mixture was purged with nitrogen for 3 min, then pressurized to 15 atm with carbon monoxide at 70° C. for 3 hrs. The reaction mixture was cooled and filtered. The filtrate was concentrated and the residue purified by silica gel column chromatography, eluting with PE:EA (4:1) to afford methyl 2-amino-3,5-difluoropyridine-4-carboxylate (260 mg, 55% yield) as a yellow solid.

LCMS (ES, m/z): [M+H]⁺: 189

### Synthesis of 56-b: (2-amino-3,5-difluoropyridin-4-yl)methanol

To a stirred solution of methyl 2-amino-3,5-difluoropyridine-4-carboxylate (250 mg, 1.3 mmol, 1 equiv) in THE (3 mL) was added LAH (76 mg, 2 mmol, 1.5 equiv) in portions at 0° C. The resulting solution was stirred for 1 h at 0° C., then quenched with HOAc (0.5 mL) and concentrated. The residue was purified by Prep-HPLC with following conditions: Column, welch Vltimate XB—C18, 50×250 mm, 10 μm; Mobile Phase: 5-30% MeCN/0.05% aqueous ammonia; to afford (2-amino-3,5-difluoropyridin-4-yl)methanol (120 mg, 56% yield) as a white solid.

LCMS (ES, m/z): [M+H]⁺: 161

### Synthesis of 56-c: 3,5-difluoro-4-([[3-methyl-1-(oxan-2-yl)pyrazolo[3,4-b]pyridin-5-yl]oxy]methyl) pyridin-2-amine

To a stirred mixture of 3-methyl-1-(oxan-2-yl)pyrazolo[3,4-b]pyridin-5-ol (110 mg, 0.47 mmol, 1 equiv) and (2-amino-3,5-difluoropyridin-4-yl)methanol (90 mg, 0.56 mmol, 1.2 equiv) in THE (3 mL) were added PPh₃ (148 mg, 0.56 mmol, 1.2 equiv) and TMAD (98 mg, 0.56 mmol, 1.2 equiv) at room temperature. The resulting solution was stirred for 2 h then diluted with water (10 mL). This mixture was extracted with EA (3×10 mL), and the combined organics were washed with brine (2×5 mL), dried over anhydrous Na₂SO₄ and concentrated. The residue was purified by Prep-HPLC with following conditions: Column, welch Vltimate XB—C18, 50×250 mm, 10 μm; Mobile Phase: 20-60% MeCN/0.1% aqueous TFA; to give 3,5-difluoro-4-([[3-methyl-1-(oxan-2-yl)pyrazolo[3,4-b]pyridin-5-yl]oxy]methyl)pyridin-2-amine (130 mg, 73% yield) as a white solid.

LCMS (ES, m/z): [M+H]⁺: 376

### Synthesis of 56-d: N-[3,5-difluoro-4-([[3-methyl-1-(oxan-2-yl)pyrazolo[3,4-b]pyridin-5-yl]oxy]methyl)pyridin-2-yl]-S-(5-fluoro-2-methoxypyridin-3-yl)methanesulfinamide

To a stirred mixture of 3,5-difluoro-4-([[3-methyl-1-(oxan-2-yl)pyrazolo[3,4-b]pyridin-5-yl]oxy]methyl)pyridin-2-amine (40 mg, 0.11 mmol, 1 equiv) in pyridine (2 mL) was added 5-fluoro-2-methoxypyridine-3-sulfonyl chloride (36 mg, 0.16 mmol, 1.5 equiv) at room temperature. The reaction mixture was stirred for 3 day at 40° C. then concentrated. The residue was purified by Prep-HPLC with following conditions: Column, welch Vltimate XB—C18, 50×250 mm, 10 μm; Mobile Phase: 30-80%/0.1% aqueous formic acid; to afford N-[3,5-difluoro-4-([[3-methyl-1-(oxan-2-yl)pyrazolo[3,4-b]pyridin-5-yl]oxy]methyl)pyridin-2-yl]-S-(5-fluoro-2-methoxypyridin-3-yl)methanesulfinamide (24 mg, 40% yield) as a white solid.

LCMS (ES, m/z): [M+H]⁺: 565

### Synthesis of Compound 56: N-[3,5-difluoro-4-[([3-methyl-1H-pyrazolo[3,4-b]pyridin-5-yl]oxy)methyl]pyridin-2-yl]-5-fluoro-2-methoxypyridine-3-sulfonamide

A mixture of N-[3,5-difluoro-4-([[3-methyl-1-(oxan-2-yl)pyrazolo[3,4-b]pyridin-5-yl]oxy]methyl)pyridin-2-yl]-5-fluoro-2-methoxypyridine-3-sulfonamide (22 mg, 39 μmol, 1 equiv) in 4M HCl in MeOH (1 mL) was stirred for 0.5 h then concentrated. The residue was purified by Prep-HPLC with following conditions: Column, welch Vltimate XB—C18, 50×250 mm, 10 μm; Mobile Phase: 30-60% MeCN/0.1% aqueous formic acid; to afford N-[3,5-difluoro-4-[([3-methyl-1H-pyrazolo[3,4-b]pyridin-5-yl]oxy)methyl]pyridin-2-yl]-5-fluoro-2-methoxypyridine-3-sulfonamide (8.7 mg, 46% yield) as a white solid.

LCMS (ES, m/z): [M+H]⁺: 481
¹H NMR (300 MHz, DMSO-d₆) δ 13.12 (s, 1H), 11.44 (s, 1H), 8.46 (d, J=2.9 Hz, 1H), 8.25 (d, J=2.7 Hz, 1H), 8.20 (s, 1H), 8.13 (dd, J=7.5, 3.1 Hz, 1H), 7.90 (d, J=2.8 Hz, 1H), 5.28 (s, 2H), 3.90 (s, 3H), 2.48 (s, 3H).

### Example 68: Synthesis of N-[2,4-difluoro-3-[([3-methyl-1H-pyrazolo[3,4-b]pyridin-5-yl]methyl)amino]phenyl]-5-fluoro-2-methoxypyridine-3-sulfonamide (Compound 57)

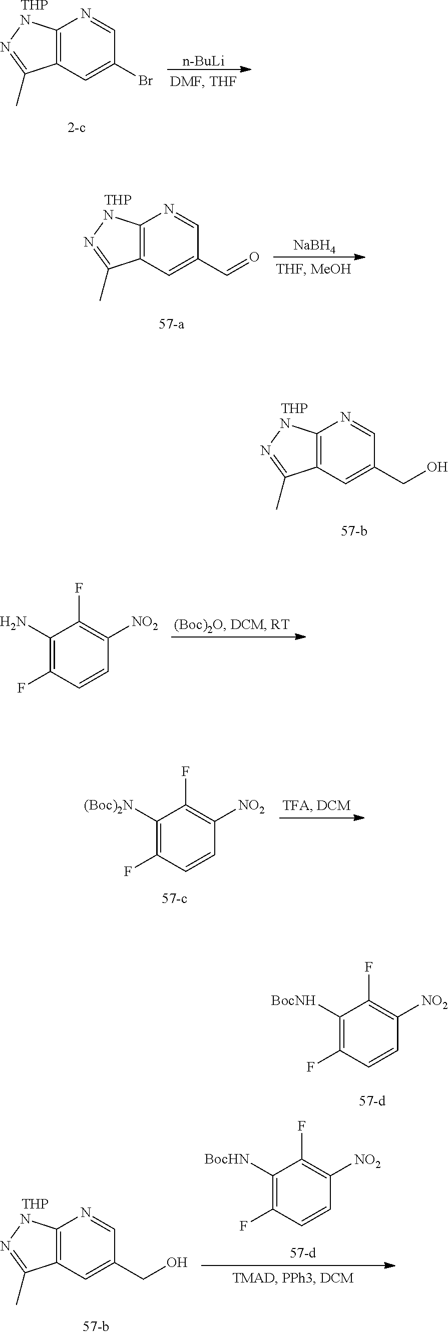

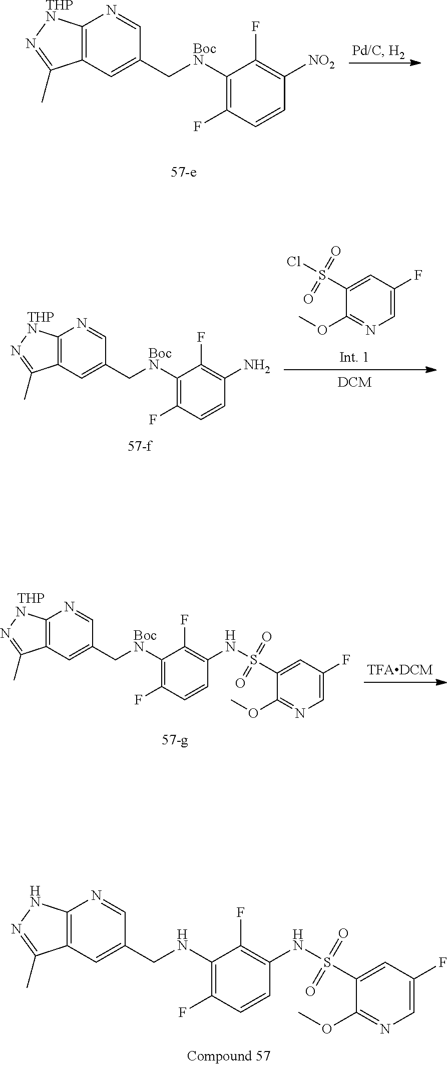

### Synthesis of 57-a. 3-methyl-1-(oxan-2-yl)pyrazolo[3,4-b]pyridine-5-carbaldehyde

To a solution of 5-bromo-3-methyl-1-(oxan-2-yl)pyrazolo[3,4-b]pyri dine (3 g, 10 mmol, 1 equiv) in THF (60 mL) at −78° C. was added 2.5 M n-BuLi in hexanes (5.3 mL, 13 mmol, 1.3 equiv) dropwise, and the resulting solution was stirred for 1 h at −78° C. DMF (1.5 g, 20 mmol, 2 equiv) was added dropwise at the same temperature, and the mixture stirred to room temperature over 3 h. The reaction was quenched with 4 M HCl in dioxane to pH 6, and water (100 mL) was added. The resulting solution was extracted with 3×50 mL of ethyl acetate. The organics were washed with 50 ml of water and 50 mL of brine, then dried (MgSO₄) and concentrated. The residue was purified by silica gel column chromatography, eluting with PE:EA (1:1) to afford 3-methyl-1-(oxan-2-yl)pyrazolo[3,4-b]pyridine-5-carbaldehyde (1.7 g, 68% yield) as a light yellow solid.

LCMS (ES, m/z): [M+H]⁺: 246

### Synthesis of 57-b: [3-methyl-1-(oxan-2-yl)pyrazolo[3,4-b]pyridin-5-yl]methanol

Into a 100 mL 3-necked round-bottom flask in an ice bath was placed 3-methyl-1-(oxan-2-yl)pyrazolo[3,4-b]pyridine-5-carbaldehyde (1.5 g, 6.1 mmol, 1 equiv), THF (30 mL), MeOH (6 mL) and NaBH₄ (0.28 g, 7.4 mmol, 1.2 equiv). The resulting solution was stirred for 5 h at 0° C. The reaction was quenched by 20 mL of MeOH and concentrated. The residue was purified by silica gel column chromatography, eluting with PE:EA (1:1) to afford [3-methyl-1-(oxan-2-yl)pyrazolo[3,4-b]pyridin-5-yl]methanol (1 g, 66% yield) as a light yellow solid.

LCMS (ES, m/z): [M+H]⁺: 248

### Synthesis of 57-c: tert-butyl N-(tert-butoxycarbonyl)-N-(2,6-difluoro-3-nitrophenyl)carbamate

Into a 100 mL 3-necked round-bottom flask was placed 2,6-difluoro-3-nitroaniline (2 g, 11.5 mmol, 1 equiv), DCM (40 mL), Boc₂O (5.5 g, 25 mmol, 2.2 equiv), TEA (3.5 g, 35 mmol, 3.0 equiv) and DMAP (0.28 g, 2.3 mmol, 0.2 equiv). The resulting solution was stirred for 1 h then quenched by the addition of 100 mL of water. This was extracted with 3×30 mL of dichloromethane, and the organics were washed with 50 ml of water and 50 mL of brine. The organic solution was dried over anhydrous sodium sulfate and concentrated. The residue was purified by silica gel column chromatography, eluting with PE:EA (3:1) to afford tert-butyl N-(tert-butoxycarbonyl)-N-(2,6-difluoro-3-nitrophenyl)carbamate (3.9 g, 91% yield) as a white solid.

### Synthesis of 57-d: tert-butyl N-(2,6-difluoro-3-nitrophenyl)carbamate

Into a 250 mL 3-necked round-bottom flask was placed tert-butyl N-(tert-butoxycarbonyl)-N-(2,6-difluoro-3-nitrophenyl)carbamate (4.6 g, 12 mmol, 1 equiv) and DCM (90 mL). This was followed by the addition of TFA (3.7 mL, 49 mmol, 4 equiv) dropwise with stirring at 0° C. The reaction was stirred for 2 h in an ice bath, then was quenched by the addition of 150 mL of saturated aqueous NaHCO₃. The resulting solution was extracted with 3×100 mL of dichloromethane, and the organics washed with 150 ml of water and 150 mL of brine. After drying over anhydrous sodium sulfate, it was concentrated. The residue was purified by silica gel column chromatography, eluting with PE:EA (4:1) to afford tert-butyl N-(2,6-difluoro-3-nitrophenyl)carbamate (2.8 g, 83% yield) as a light yellow solid.

LCMS (ES, m/z): [M+H]⁺: 275

### Synthesis of 57-e: tert-butyl N-(2,6-difluoro-3-nitrophenyl)-N-[[3-methyl-1-(oxan-2-yl)pyrazolo[3,4-b]pyridin-5-yl]methyl]carbamate

Into a 100 mL 3-necked round-bottom flask was placed tert-butyl N-(2,6-difluoro-3-nitrophenyl)carbamate (1.06 g, 3.9 mmol, 1.2 equiv), DCM (30 mL), [3-methyl-1-(oxan-2-yl)pyrazolo[3,4-b]pyridin-5-yl]methanol (0.8 g, 3.2 mmol, 1 equiv), TMAD (0.84 g, 4.9 mmol, 1.5 equiv) and PPh₃ (1.27 g, 4.9 mmol, 1.5 equiv). The resulting solution was stirred overnight then concentrated. The residue was purified by silica gel column chromatography, eluting with PE:EA (3:1) to afford tert-butyl N-(2,6-difluoro-3-nitrophenyl)-N-[[3-methyl-1-(oxan-2-yl)pyrazolo[3,4-b]pyridin-5-yl]methyl]carbamate (1.5 g, 92% yield) as a white solid.

LCMS (ES, m/z): [M+H]⁺: 504

### Synthesis of 57-f: tert-butyl N-(3-amino-2,6-difluorophenyl)-N-[[3-methyl-1-(oxan-2-yl)pyrazolo[3,4-b]pyridin-5-yl]methyl]carbamate

Into a 250 mL round-bottom flask was placed tert-butyl N-(2,6-difluoro-3-nitrophenyl)-N-[[3-methyl-1-(oxan-2-yl)pyrazolo[3,4-b]pyridin-5-yl]methyl]carbamate (1.9 g, 3.8 mmol, 1 equiv) and THE (50 mL). To the resulting solution was added 10% Pd/C (100 mg) and the mixture stirred for 2 h under 2 atm of hydrogen. The solids were removed by filtration and the filtrate concentrated to give tert-butyl N-(3-amino-2,6-difluorophenyl)-N-[[3-methyl-1-(oxan-2-yl)pyrazolo[3,4-b]pyridin-5-yl]methyl]carbamate (1.45 g, 80% yield) as an off-white solid.

LCMS (ES, m/z): [M+H]⁺: 474

### Synthesis of 57-g: tert-butyl N-[2,6-difluoro-3-(5-fluoro-2-methoxypyridine-3-sulfonamido)phenyl]-N-[[3-methyl-1-(oxan-2-yl)pyrazolo[3,4-b]pyridin-5-yl]methyl]carbamate

Into a 40 mL vial was placed tert-butyl N-(3-amino-2,6-difluorophenyl)-N-[[3-methyl-1-(oxan-2-yl)pyrazolo[3,4-b]pyridin-5-yl]methyl]carbamate (150 mg, 0.32 mmol, 1 equiv), DCM (4 mL), pyridine (75 mg, 0.95 mmol, 3 equiv) and 5-fluoro-2-methoxypyridine-3-sulfonyl chloride (100 mg, 0.44 mmol, 1.4 equiv). The resulting solution was stirred overnight then concentrated. The crude product was purified by Flash-Prep-HPLC with the following conditions: Column, WelFlash TM C18-I, Spherical C18 20-40 μm, 120 g; mobile phase: 5-90% MeCN/0.1% aqueous formic acid; Detector, 220 nm; to give tert-butyl N-[2,6-difluoro-3-(5-fluoro-2-methoxypyridine-3-sulfonamido)phenyl]-N-[[3-methyl-1-(oxan-2-yl)pyrazolo[3,4-b]pyridin-5-yl]methyl]carbamate (100 mg, 48% yield) as an off-white solid.

LCMS (ES, m/z): [M+H]⁺: 663

### Synthesis of Compound 57: N-[2,4-difluoro-3-[([3-methyl-1H-pyrazolo[3,4-b]pyridin-5-yl]methyl)amino]phenyl]-5-fluoro-2-methoxypyridine-3-sulfonamide

Into a 40 mL vial was placed tert-butyl N-[2,6-difluoro-3-(5-fluoro-2-methoxypyridine-3-sulfonamido)phenyl]-N-[[3-methyl-1-(oxan-2-yl)pyrazolo[3,4-b]pyridin-5-yl]methyl]carbamate (90 mg, 0.14 mmol, 1 equiv), DCM (3 mL) and TFA (1 mL). The resulting solution was stirred for 3 h then concentrated. The residue was purified by Prep-HPLC with the following conditions: Column, welch Vltimate XB—C18, 50×250 mm, 10 μm; Mobile Phase: 20-50% MeCN/0.1% aqueous formic acid; Detector, 220 nm; to give N-[2,4-difluoro-3-[([3-methyl-1H-pyrazolo[3,4-b]pyridin-5-yl]methyl)amino]phenyl]-5-fluoro-2-methoxypyridine-3-sulfonamide (60 mg, 92% yield) as a white solid.

LCMS (ES, m/z): [M+H]⁺: 479
¹H NMR (300 MHz, DMSO-d₆) δ 13.12 (s, 1H), 10.01 (s, 1H), 8.35 (dd, J=5.5, 2.5 Hz, 2H), 7.97 (d, J=2.0 Hz, 1H), 7.85 (dd, J=7.3, 3.0 Hz, 1H), 6.88-6.74 (m, 1H), 6.49-6.37 (m, 1H), 5.97 (s, 1H), 4.42 (s, 2H), 3.87 (s, 3H), 2.45 (s, 3H).

### Example 69: Synthesis of 5-chloro-N-[2,4-difluoro-3-[([3-methyl-1H-pyrazolo[3,4-b]pyridin-5-yl]methyl)amino]phenyl]-2-methoxypyridine-3-sulfonamide (Compound 58)

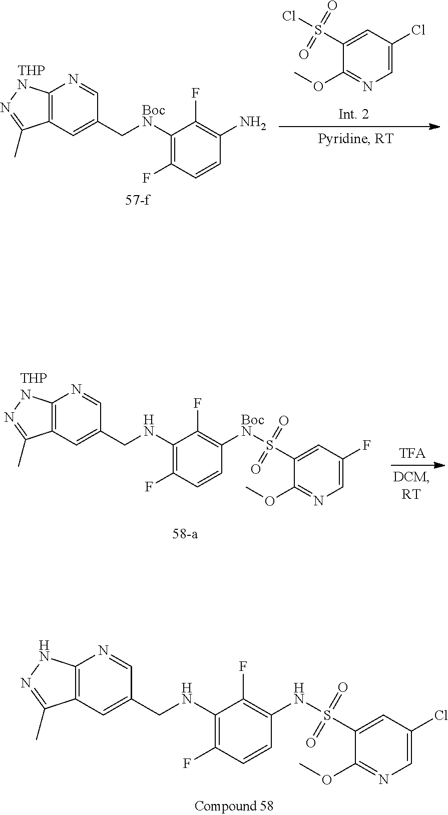 [SMILES: Cc1n[nH]c2ncc(CN(C(=O)OC(C)(C)C)c3c(F)ccc(N)c3F)cc12 ; COc1ncc(Cl)cc1S(=O)(=O)Cl ; COc1ncc(F)cc1S(=O)(=O)N(C(=O)OC(C)(C)C)c1ccc(F)c(NCc2cnc3[nH]nc(C)c3c2)c1F ; COc1ncc(Cl)cc1S(=O)(=O)Nc1ccc(F)c(NCc2cnc3[nH]nc(C)c3c2)c1F ; "THP" ; "THP"]

### Synthesis of 58-a: tert-butyl N-(5-chloro-2-methoxypyridin-3-ylsulfonyl)-N-[2,4-difluoro-3-([[3-methyl-1-(oxan-2-yl)pyrazolo[3,4-b]pyridin-5-yl]methyl]amino)phenyl]carbamate

Into a 40 mL vial was placed tert-butyl N-(3-amino-2,6-difluorophenyl)-N-[[3-methyl-1-(oxan-2-yl)pyrazolo[3,4-b]pyridin-5-yl]methyl]carbamate (150 mg, 0.32 mmol, 1 equiv), DCM (4 mL), pyridine (75 mg, 0.95 mmol, 3 equiv) and 5-chloro-2-methoxypyridine-3-sulfonyl chloride (107 mg, 0.44 mmol, 1.4 equiv). The resulting solution was stirred overnight then concentrated under vacuum. The crude product was purified by Flash-Prep-HPLC with the following conditions: Column, WelFlash TM C18-I, Spherical C18 20-40 μm, 120 g; mobile phase: 20-90% MeCN/0.1% aqueous formic acid; Detector, 220 nm; to give tert-butyl N-(5-chloro-2-methoxypyridin-3-ylsulfonyl)-N-[2,4-difluoro-3-([[3-methyl-1-(oxan-2-yl)pyrazolo[3,4-b]pyridin-5-yl]methyl]amino)phenyl]carbamate (120 mg, 56% yield) as a white solid.

LCMS (ES, m/z): [M+H]⁺: 679

### Synthesis of Compound 58: 5-chloro-N-[2,4-difluoro-3-[([3-methyl-1H-pyrazolo[3,4-b]pyridin-5-yl]methyl)amino]phenyl]-2-methoxypyridine-3-sulfonamide

Into an 8 mL vial was placed tert-butyl N-(5-chloro-2-methoxypyridin-3-ylsulfonyl)-N-[2,4-difluoro-3-([[3-methyl-1-(oxan-2-yl)pyrazolo[3,4-b]pyridin-5-yl]methyl]amino)phenyl]carbamate (90 mg, 0.13 mmol, 1 equiv), DCM (3 mL) and TFA (1 mL). The resulting solution was stirred for 3 h then concentrated under vacuum. The crude product was purified by Prep-HPLC with the following conditions: Column, welch Vltimate XB—C18, 50×250 mm, 10 μm; Mobile Phase: 25-55% MeCN/0.1% aqueous formic acid; Detector, 220 nm; to give 5-chloro-N-[2,4-difluoro-3-[([3-methyl-1H-pyrazolo[3,4-b]pyridin-5-yl]methyl)amino]phenyl]-2-methoxypyridine-3-sulfonamide (64 mg, 98% yield) as a white solid.

LCMS (ES, m/z): [M+H]⁺: 495
¹H NMR (300 MHz, DMSO-d₆) δ 13.11 (s, 1H), 10.04 (s, 1H), 8.37 (dd, J=2.5, 1.4 Hz, 2H), 7.97 (d, J=2.0 Hz, 1H), 7.91 (d, J=2.6 Hz, 1H), 6.88-6.75 (m, 1H), 6.51-6.37 (m, 1H), 5.98 (s, 1H), 4.42 (d, J=7.1 Hz, 2H), 3.87 (s, 3H), 2.45 (s, 3H).

### Example 70: Synthesis of N-[2,4-difluoro-3-[([3-methyl-1H-pyrazolo[3,4-b]pyridin-5-yl]methyl)amino]phenyl]-5-fluoro-2-methylpyridine-3-sulfonamide (Compound 59)

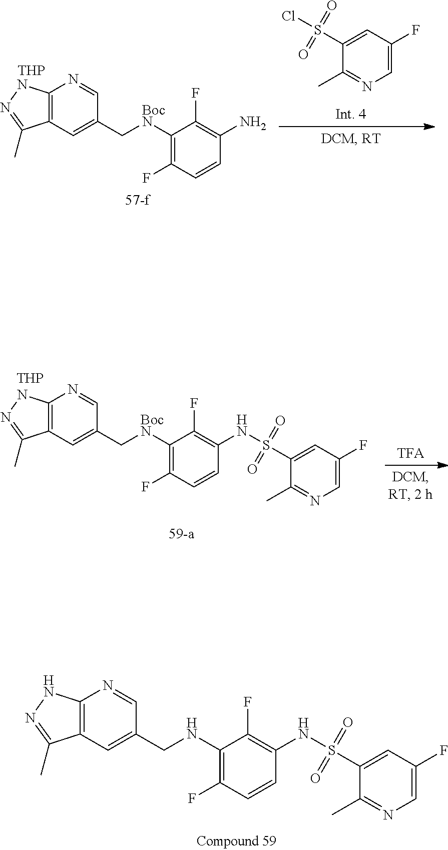 [SMILES: Cc1n[nH]c2ncc(CN(C(=O)OC(C)(C)C)c3c(F)ccc(N)c3F)cc12 ; Cc1ncc(F)cc1S(=O)(=O)Cl ; Cc1ncc(F)cc1S(=O)(=O)Nc1ccc(F)c(N(Cc2cnc3[nH]nc(C)c3c2)C(=O)OC(C)(C)C)c1F ; Cc1ncc(F)cc1S(=O)(=O)Nc1ccc(F)c(NCc2cnc3[nH]nc(C)c3c2)c1F ; "THP" ; "THP"]

### Synthesis of 59-a: tert-butyl N-[2,6-difluoro-3-(5-fluoro-2-methylpyridine-3-sulfonamido)phenyl]-N-[[3-methyl-1-(oxan-2-yl)pyrazolo[3,4-b]pyridin-yl]methyl]carbamate

Into a 40 mL vial was placed tert-butyl N-(3-amino-2,6-difluorophenyl)-N-[[3-methyl-1-(oxan-2-yl)pyrazolo[3,4-b]pyridin-5-yl]methyl]carbamate (150 mg, 0.32 mmol, 1 equiv), DCM (4 mL), pyridine (75 mg, 0.95 mmol, 3 equiv) and 5-fluoro-2-methylpyridine-3-sulfonyl chloride (93 mg, 0.44 mmol, 1.4 equiv). The resulting solution was stirred overnight then concentrated under vacuum. The residue was purified by Flash-Prep-HPLC with the following conditions: Column, WelFlash TM C18-I, Spherical C18 20-40 μm, 120 g; mobile phase: 20-90% MeCN/0.1% aqueous formic acid; Detector, 220 nm; to give tert-butyl N-[2,6-difluoro-3-(5-fluoro-2-methylpyridine-3-sulfonamido)phenyl]-N-[[3-methyl-1-(oxan-2-yl)pyrazolo[3,4-b]pyridin-yl]methyl]carbamate (75 mg, 37% yield) as a white solid.

LCMS (ES, m/z): [M+H]⁺: 647

### Synthesis of Compound 59: N-[2,4-difluoro-3-[([3-methyl-1H-pyrazolo[3,4-b]pyridin-5-yl]methyl)amino]phenyl]-5-fluoro-2-methylpyridine-3-sulfonamide

Into a 40 mL vial was placed tert-butyl N-[2,6-difluoro-3-(5-fluoro-2-methylpyridine-3-sulfonamido)phenyl]-N-[[3-methyl-1-(oxan-2-yl)pyrazolo[3,4-b]pyridin-5-yl]methyl]carbamate (75 mg, 0.12 mmol, 1 equiv), DCM (3 mL) and TFA (1 mL). The resulting solution was stirred for 3 h, then concentrated under vacuum. The crude product was purified by Prep-HPLC with the following conditions Column, welch Vltimate XB—C18, 50×250 mm, 10 μm; Mobile Phase: 25-55% MeCN/0.1% aqueous formic acid; Detector, 220 nm; to give N-[2,4-difluoro-3-[([3-methyl-1H-pyrazolo[3,4-b]pyridin-5-yl]methyl)amino]phenyl]-5-fluoro-2-methylpyridine-3-sulfonamide (40 mg, 75% yield) as a white solid.

LCMS (ES, m/z): [M+H]⁺: 463
¹H NMR (300 MHz, DMSO-d₆) δ 13.11 (s, 1H), 10.04 (s, 1H), 8.37 (dd, J=2.5, 1.4 Hz, 2H), 7.97 (d, J=2.0 Hz, 1H), 7.91 (d, J=2.6 Hz, 1H), 6.88-6.75 (m, 1H), 6.51-6.37 (m, 1H), 5.98 (s, 1H), 4.42 (d, J=7.1 Hz, 2H), 3.87 (s, 3H), 2.45 (s, 3H).

### Example 71: Synthesis of 5-chloro-N-(2,4-difluoro-3-((imidazo[1,5-b]pyridazin-3-yloxy)methyl)phenyl)-2-methoxypyridine-3-sulfonamide (Compound 60)

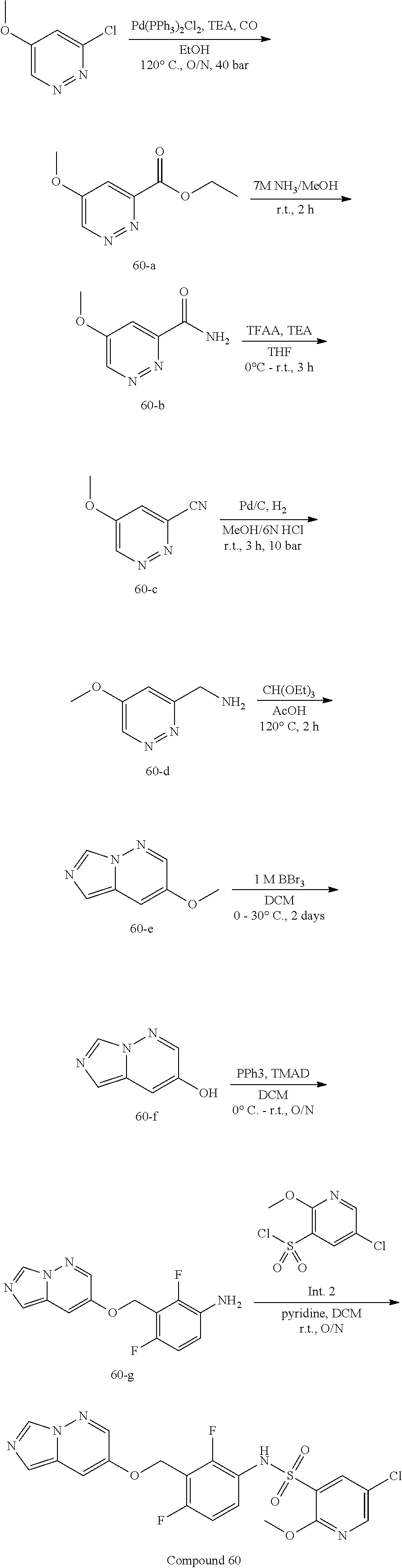

### Synthesis of 60-a: ethyl 5-methoxypyridazine-3-carboxylate

To a solution of 3-chloro-5-methoxypyridazine (16 g, 110 mmol, 1 equiv) in EtOH (190 mL) was added Pd(PPh₃)₂Cl₂ (7.8 g, 11 mmol, 0.1 equiv) and TEA (16.8 g, 166 mmol, 1.5 equiv) in a pressure tank. The mixture was purged with nitrogen, then pressurized to 40 bar with carbon monoxide. The reaction was stirred at 120° C. overnight, then cooled and filtered. The filtrate was concentrated and the residue purified by silica gel column chromatography, eluting with PE:EA (1:1) to afford ethyl 5-methoxypyridazine-3-carboxylate (8 g, 40% yield) as a yellow solid.

LCMS (ES, m/z): [M+H]⁺: 183

### Synthesis of 60-b: 5-methoxypyridazine-3-carboxamide

Ethyl 5-methoxypyridazine-3-carboxylate (9 g, 49 mmol, 1 equiv) was dissolved in 7 M NH₃/MeOH (45 mL) and stirred at room temperature for 2 h. The reaction solution was concentrated under vacuum directly and the residue was triturated with DCM (50 mL). The precipitated solids were collected by filtration, washed with DCM (50 mL) and dried to afford 5-methoxypyridazine-3-carboxamide (6 g, 79% yield) as a yellow solid.

LCMS (ES, m/z): [M+H]⁺: 154

### Synthesis of 60-c: 5-methoxypyridazine-3-carbonitrile

To a stirred solution of 5-methoxypyridazine-3-carboxamide (6 g, 39 mmol, 1 equiv) in THE (120 mL) was added TFAA (16 g, 78 mmol, 2 equiv) and TEA (10 g, 98 mmol, 2.5 equiv) dropwise at 0° C. The reaction was stirred at room temperature for 3 h, then concentrated. The residue was diluted with H₂O (10 mL) and saturated NaHCO₃ (15 mL), then extracted with EA (3×20 mL). The combined organics were washed with brine (3×10 mL), dried over anhydrous Na₂SO₄ and concentrated. The residue was purified by silica gel column chromatography, eluting with PE:EA (2:1) to afford 5-methoxypyridazine-3-carbonitrile (5 g, 94% yield) as a yellow solid.

LCMS (ES, m/z): [M+H]⁺: 136

### Synthesis of 60-d: 1-(5-methoxypyridazin-3-yl)methanamine

To a solution of 5-methoxypyridazine-3-carbonitrile (5.4 g, 40 mmol, 1 equiv) in MeOH (500 mL) was added Pd/C (10%, 0.54 g) and 6 N HCl (5 mL) in a pressure tank. The mixture was hydrogenated at room temperature under 10 bar of hydrogen pressure for 3 h. After completion, the reaction solution was filtered through a Celite pad and concentrated under reduced pressure to afford 1-(5-methoxypyridazin-3-yl)methanamine (5 g, crude) as a red solid, which was used for next step directly without further purification.

LCMS (ES, m/z): [M+H]⁺: 140

### Synthesis of 60-e: 3-methoxyimidazo[1,5-b]pyridazine

1-(5-Methoxypyridazin-3-yl)methanamine (4 g, 28.7 mmol, 1 equiv), (diethoxymethoxy)ethane (30 mL) and AcOH (7.5 mL) were stirred at 120° C. for 2 h in a sealed tube. The reaction solution was cooled and concentrated, and the residue diluted with H₂O (10 mL). The mixture was basified to pH 8 with saturated NaHCO₃ aqueous, then extracted with EA (3×10 mL). The combined organics were washed with brine (2×5 mL), dried over anhydrous Na₂SO₄ and concentrated under reduced pressure. The residue was purified by silica gel column chromatography, eluting with PE:EA (1:2) to afford 3-methoxyimidazo[1,5-b]pyridazine (1 g, 23% yield) as a yellow solid.

LCMS (ES, m/z): [M+H]⁺: 150

### Synthesis of 60-f: imidazo[1,5-b]pyridazin-3-ol

To a stirred solution of 3-methoxyimidazo[1,5-b]pyridazine (1.1 g, 7.4 mmol, 1 equiv) in DCM (14 mL) was added 1M BBr₃ (44 mL, 44 mmol, 6 equiv) dropwise at 0° C., and the mixture stirred at 30° C. for 2 days. Stirring was stopped and the mixture allowed to stand for 30 mins. The supernatant liquid was removed by decanting, and the precipitate diluted with DCM (20 mL). MeOH (10 mL) was added dropwise at 0° C. until all the precipitate was dissolved. The resulting solution was concentrated and the residue triturated with MTBE (10 mL). The precipitated solids were collected by filtration, washed with MTBE (1×10 mL) and dried to give imidazo[1,5-b]pyridazin-3-ol (300 mg, 30% yield) as a grey solid.

LCMS (ES, m/z): [M+H]⁺: 136

### Synthesis of 60-g: 2,4-difluoro-3-((imidazo[1,5-b]pyridazin-3-yloxy)methyl)aniline

To a stirred solution of imidazo[1,5-b]pyridazin-3-ol (180 mg, 1.3 mmol, 1 equiv) in DCM (18 mL) was added (3-amino-2,6-difluorophenyl)methanol (233 mg, 1.4 mmol, 1.1 equiv) and PPh₃ (384 mg, 1.4 mmol, 1.1 equiv). The solution was cooled to 0° C. and TMAD (252 mg, 1.4 mmol, 1.1 equiv) in DCM (5 mL) was added dropwise. The reaction was stirred at room temperature overnight. The resulting mixture was diluted with H₂O (5 mL) and extracted with DCM (3×5 mL). The combined organics were washed with brine (2×5 mL), dried over anhydrous Na₂SO₄ and concentrated under reduced pressure. The residue was purified by silica gel column chromatography, eluting with PE:EA (1:1) to afford 2,4-difluoro-3-([imidazo[1,5-b]pyridazin-3-yloxy]methyl)aniline (160 mg, 44% yield) as a red solid.

LCMS (ES, m/z): [M+H]⁺: 277

### Synthesis of Compound 60: 5-chloro-N-(2,4-difluoro-3-((imidazo[1,5-b]pyridazin-3-yloxy)methyl)phenyl)-2-methoxypyridine-3-sulfonamide

To a stirred solution of 2,4-difluoro-3-([imidazo[1,5-b]pyridazin-3-yloxy]methyl)aniline (150 mg, 0.54 mmol, 1 equiv) in DCM (3 mL) was added 5-chloro-2-methoxypyridine-3-sulfonyl chloride (144 mg, 0.6 mmol, 1.1 equiv) and pyridine (129 mg, 1.6 mmol, 3 equiv). The reaction was stirred at room temperature overnight. The resulting solution was concentrated and the residue was purified by prep-HPLC with the following conditions: Column, welch Vltimate XB—C18, 50×250 mm, 10 μm; Mobile Phase: 35-85% MeCN/0.1% aqueous formic acid; Detector, 220 nm; to afford 5-chloro-N-[2,4-difluoro-3-([imidazo[1,5-b]pyridazin-3-yloxy]methyl)phenyl]-2-methoxypyridine-3-sulfonamide (50 mg, 19% yield) as a white solid.

LCMS (ES, m/z): [M+H]⁺: 482
¹H NMR (300 MHz, DMSO-d₆) δ 8.51 (s, 1H), 8.44 (d, J=2.6 Hz, 1H), 8.11 (d, J=2.8 Hz, 1H), 8.03 (d, J=2.6 Hz, 1H), 7.58 (d, J=2.9 Hz, 1H), 7.38 (td, J=9.0, 6.0 Hz, 1H), 7.24 (s, 1H), 7.13 (t, J=9.0 Hz, 1H), 5.11 (s, 2H), 3.90 (s, 3H).

### Example 72: Synthesis of N-[2-chloro-4-fluoro-3-[([3-methyl-1H-pyrazolo[3,4-b]pyridin-5-yl]oxy)methyl]phenyl]-5-fluoro-2-methoxypyridine-3-sulfonamide (Compound 61)

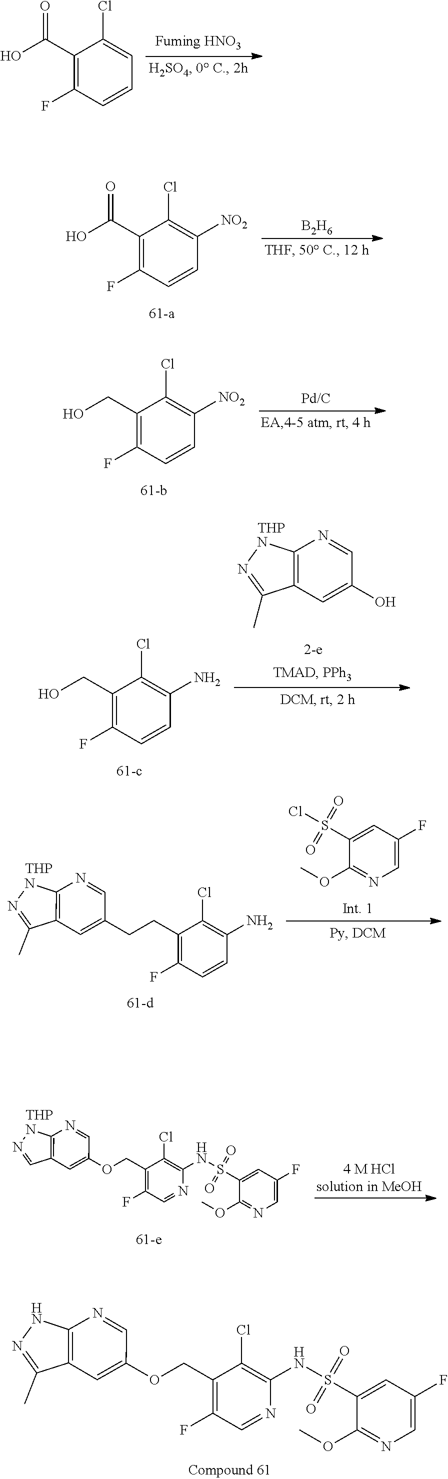

### Synthesis of 61-a: 2-chloro-6-fluoro-3-nitrobenzoic acid

To a stirred solution of 2-chloro-6-fluorobenzoic acid (10 g, 57 mmol, 1 equiv) in H₂SO₄ (200 mL) was added HNO₃ (4 g, 63 mmol, 1.1 equiv, 98%) dropwise at 0° C. The resulting mixture was stirred for 2 h at low temperature, then poured into ice-water (1 L) and extracted with EA (3×200 mL). The combined organics were washed with brine (3×100 mL), dried over anhydrous Na₂SO₄ and concentrated under reduced pressure. The residue was purified by silica gel column chromatography, eluting with EA:MeOH (10:1) to afford 2-chloro-6-fluoro-3-nitrobenzoic acid (8.2 g, 65% yield) as a light yellow solid.

### Synthesis of 61-b: (2-chloro-6-fluoro-3-nitrophenyl)methanol

A solution of 2-chloro-6-fluoro-3-nitrobenzoic acid (5 g) in BH₃-THF (100 mL, 1 mol/L in THF) was stirred for 12 h at 50° C. The reaction was quenched by the addition of MeOH (100 mL) dropwise at room temperature. The resulting solution was concentrated under reduced pressure and the residue purified by silica gel column chromatography, eluting with PE:EA (4:1) to afford (2-chloro-6-fluoro-3-nitrophenyl)methanol (3.7 g, 79% yield) as a light yellow solid.

LCMS (ES, m/z): [M+H]⁺: 206

### Synthesis of 61-c: (3-amino-2-chloro-6-fluorophenyl)methanol

To a solution of (2-chloro-6-fluoro-3-nitrophenyl)methanol (1.6 g, 7.7 mmol, 1 equiv) in EA (30 mL) was added Pd/C (10%, 80 mg) under nitrogen atmosphere. The mixture was hydrogenated at 4 atm for 4 h under hydrogen atmosphere, then filtered through a Celite pad and concentrated under reduced pressure. The residue was purified by silica gel column chromatography, eluting with PE:EA (2:1) to afford (3-amino-2-chloro-6-fluorophenyl)methanol (300 mg, 22% yield) as a yellow solid.

LCMS (ES, m/z): [M+H]⁺: 176

### Synthesis of 61-d: 2-chloro-4-fluoro-3-([[3-methyl-1-(oxan-2-yl)pyrazolo[3,4-b]pyridin-5-yl]oxy]methyl)aniline

To a solution of (3-amino-2-chloro-6-fluorophenyl)methanol (500 mg, 2.8 mmol, 1 equiv) in DCM (10 mL) were added 3-methyl-1-(oxan-2-yl)pyrazolo[3,4-b]pyridin-5-ol (664 mg, 2.8 mmol, 1 equiv), PPh₃ (1.1 g, 4.2 mmol, 1.5 equiv) and TMAD (735. mg, 4.2 mmol, 1.5 equiv) at room temperature. The resulting solution was stirred at room temperature for 2 h, then water (300 mL) was added. The mixture was extracted with ethyl acetate (3×100 mL), and the combined organics washed with brine (2×50 mL), dried over anhydrous Na₂SO₄ and concentrated under reduced pressure. The crude product was purified by column chromatography over silica gel (eluent: PE:EA=3:1) to afford 2-chloro-4-fluoro-3-([[3-methyl-1-(oxan-2-yl)pyrazolo[3,4-b]pyridin-5-yl]oxy]methyl)aniline (1.1 g, 98% yield) as a white solid.

LCMS (ES, m/z): [M+H]⁺: 391

### Synthesis of 61-e: N-[2-chloro-4-fluoro-3-([[3-methyl-1-(oxan-2-yl)pyrazolo[3,4-b]pyridin-5-yl]oxy]methyl)phenyl]-5-fluoro-2-methoxypyridine-3-sulfonamide

To a stirred solution of 2-chloro-4-fluoro-3-([[3-methyl-1-(oxan-2-yl)pyrazolo[3,4-b]pyridin-5-yl]oxy]methyl)aniline (150 mg, 0.38 mmol, 1 equiv) and pyridine (91 mg, 1.1 mmol, 3 equiv) in DCM (5 mL) were added 5-fluoro-2-methoxypyridine-3-sulfonyl chloride (103 mg, 0.46 mmol, 1.2 equiv) in portions at room temperature. The reaction mixture was stirred for 2 h then concentrated under reduced pressure. The residue was purified by silica gel column chromatography, eluting with PE:EA (2:1) to afford N-[2-chloro-4-fluoro-3-([[3-methyl-1-(oxan-2-yl)pyrazolo[3,4-b]pyridin-5-yl]oxy]methyl)phenyl]-5-fluoro-2-methoxypyridine-3-sulfonamide (200 mg, 90% yield) as a white solid.

LCMS (ES, m/z): [M+H]⁺: 566

### Synthesis of Compound 61: N-[2-chloro-4-fluoro-3-[([3-methyl-1H-pyrazolo[3,4-b]pyridin-5-yl]oxy)methyl]phenyl]-5-fluoro-2-methoxypyridine-3-sulfonamide

A mixture of N-[2-chloro-4-fluoro-3-([[3-methyl-1-(oxan-2-yl)pyrazolo[3,4-b]pyridin-5-yl]oxy]methyl)phenyl]-5-fluoro-2-methoxypyridine-3-sulfonamide (200 mg, 1 equiv) and 4M HCl in MeOH (10 mL) was stirred for 1 h at room temperature. The resulting mixture was concentrated under vacuum. The residue was purified by Prep-HPLC with the following conditions: Column, welch Vltimate XB—C18, 50×250 mm, 10 m; Mobile Phase: 30-65% MeCN 0.1% aqueous formic acid; Detector, 220 nm; to afford N-[2-chloro-4-fluoro-3-[([3-methyl-1H-pyrazolo[3,4-b]pyridin-5-yl]oxy)methyl]phenyl]-5-fluoro-2-methoxypyridine-3-sulfonamide (130 mg, 76% yield) as a white solid.

LCMS (ES, m/z): [M+H]⁺: 496
¹H NMR (300 MHz, DMSO-d₆) δ 13.09 (s, 1H), 10.35 (s, 1H), 8.46 (d, J=3.0 Hz, 1H), 8.19 (d, J=2.7 Hz, 1H), 7.97 (dd, J=7.3, 3.0 Hz, 1H), 7.89 (d, J=2.7 Hz, 1H), 7.44 (dd, J=9.0, 5.7 Hz, 1H), 7.33 (t, J=8.9 Hz, 1H), 5.19 (d, J=1.9 Hz, 2H), 3.86 (s, 3H), 2.48 (s, 3H).

### Example 73: Synthesis of N-[2-cyano-4-fluoro-3-[([3-methyl-1H-pyrazolo[3,4-b]pyridin-5-yl]oxy)methyl]phenyl]-5-fluoro-2-methoxypyridine-3-sulfonamide (Compound 62)

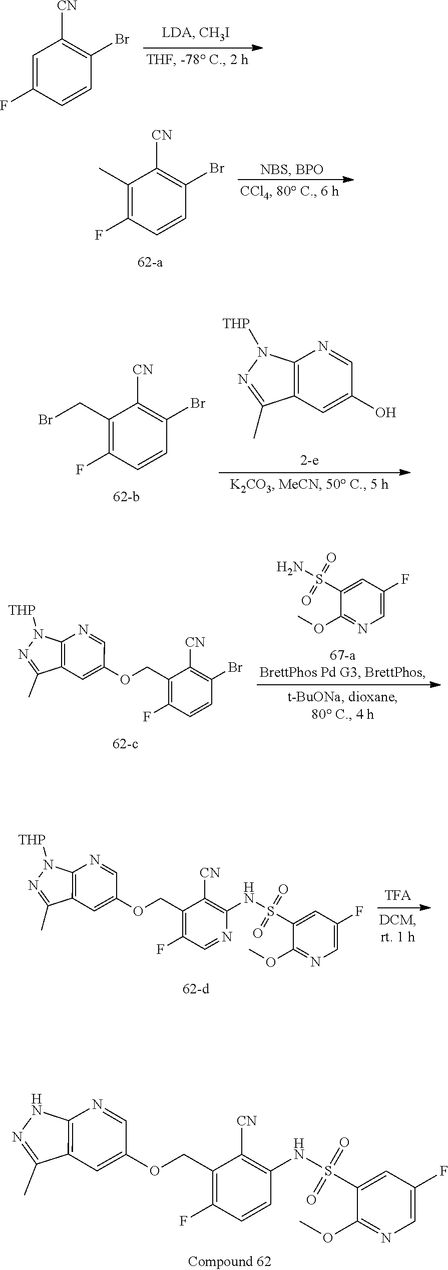

### Synthesis of 62-a: 6-bromo-3-fluoro-2-methylbenzonitrile

To a stirred solution of 2-bromo-5-fluorobenzonitrile (1 g, 5 mmol, 1 equiv) in THF (10 mL) was added LDA (3.8 mL, 7.5 mmol, 1.5 equiv, 2 M solution in THF) dropwise at −78° C. under nitrogen atmosphere. The resulting mixture was stirred for 1 h at low temperature, then CH₃I (1.42 g, 10 mmol, 2 equiv) was added dropwise over 10 min at −78° C. The resulting mixture was stirred for additional 1 h at −78° C., before being quenched with 1 M hydrochloric acid (10 mL). The mixture was extracted with EA (3×20 mL). The combined organics were washed with brine (1×20 mL), dried over anhydrous Na₂SO₄ and concentrated under reduced pressure. The residue was purified by Flash-Prep-HPLC with the following conditions: Column, WelFlash TM C18-I, Spherical C18 20-40 μm, 120 g; mobile phase: 30-70% MeCN/0.1% aqueous formic acid to afford 6-bromo-3-fluoro-2-methylbenzonitrile (408 mg, 38% yield) as a brown solid.

### Synthesis of 62-b: 6-bromo-2-(bromomethyl)-3-fluorobenzonitrile

To a stirred solution of 6-bromo-3-fluoro-2-methylbenzonitrile (380 mg, 1.8 mmol, 1 equiv) and BPO (46 mg, 0.18 mmol, 0.1 equiv) in CCl₄ (10 mL) was added NBS (474 mg, 2.7 mmol, 1.5 equiv) in portions at room temperature. The resulting mixture was stirred for 6 h at 80° C. The resulting mixture was concentrated under reduced pressure. The residue was purified by Flash-Prep-HPLC with the following conditions: Column, WelFlash TM C18-I, Spherical C18 20-40 μm, 120 g; mobile phase: 30-70% MeCN/0.1% aqueous formic acid; to afford 6-bromo-2-(bromomethyl)-3-fluorobenzonitrile (362 mg, 70% yield) as a light yellow solid.

### Synthesis of 62-c: 6-bromo-3-fluoro-2-(((3-methyl-1-(tetrahydro-2H-pyran-2-yl)-1H-pyrazolo[3,4-b]pyridin-5-yl)oxy)methyl)benzonitrile

To a stirred solution of 6-bromo-2-(bromomethyl)-3-fluorobenzonitrile (340 mg, 1.16 mmol, 1 equiv) and 3-methyl-1-(oxan-2-yl)pyrazolo[3,4-b]pyridin-5-ol (271 mg, 1.16 mmol, 1 equiv) in MeCN (10 mL) was added K₂CO₃ (481 mg, 3.48 mmol, 3 equiv) in portions at room temperature. The mixture was stirred for 5 h at 50° C. The resulting mixture was concentrated under reduced pressure. The residue was purified by Flash-Prep-HPLC with the following conditions: Column, WelFlash TM C18-I, Spherical C18 20-40 μm, 120 g; mobile phase: 30-80% MeCN/0.1% aqueous formic acid; to afford 6-bromo-3-fluoro-2-([[3-methyl-1-(oxan-2-yl)pyrazolo[3,4-b]pyridin-5-yl]oxy]methyl)benzonitrile (380 mg, 74% yield) as a white solid.

LCMS (ES, m/z): [M+H]⁺: 445

### Synthesis of 62-d: N-(2-cyano-4-fluoro-3-(((3-methyl-1-(tetrahydro-2H-pyran-2-yl)-1H-pyrazolo[3,4-b]pyridin-5-yl)oxy)methyl)phenyl)-5-fluoro-2-methoxypyridine-3-sulfonamide

To a stirred solution of 6-bromo-3-fluoro-2-([[3-methyl-1-(oxan-2-yl)pyrazolo[3,4-b]pyridin-5-yl]oxy]methyl)benzonitrile (350 mg, 0.79 mmol, 1 equiv) and 5-fluoro-2-methoxypyridine-3-sulfonamide (178 mg, 0.86 mmol, 1.1 equiv) in dioxane (10 mL) were added BrettPhos Pd G3 (214 mg, 0.24 mmol, 0.3 equiv), t-BuONa (151 mg, 1.6 mmol, 2 equiv) and BrettPhos (127 mg, 0.24 mmol, 0.3 equiv) in portions at room temperature under nitrogen atmosphere. The resulting mixture was stirred for 4 h at 80° C. then concentrated under reduced pressure to give N-[2-cyano-4-fluoro-3-([[3-methyl-1-(oxan-2-yl)pyrazolo[3,4-b]pyridin-5-yl]oxy]methyl)phenyl]-5-fluoro-2-methoxypyridine-3-sulfonamide (500 mg, crude) as a brown solid which was used in the next step directly without further purification.

LCMS (ES, m/z): [M+H]⁺: 571

### Synthesis of Compound 62: N-[2-cyano-4-fluoro-3-[([3-methyl-1H-pyrazolo[3,4-b]pyridin-5-yl]oxy)methyl]phenyl]-5-fluoro-2-methoxypyridine-3-sulfonamide

A solution of N-[2-cyano-4-fluoro-3-([[3-methyl-1-(oxan-2-yl)pyrazolo[3,4-b]pyridin-5-yl]oxy]methyl)phenyl]-5-fluoro-2-methoxypyridine-3-sulfonamide (500 mg, 0.88 mmol, 1 equiv) in TFA (1 mL) and DCM (3 mL) was stirred for 1 h at room temperature. The resulting mixture was concentrated under reduced pressure. The residue was dissolved in EA (5 mL) and water (10 mL) added. The mixture was basified to pH 8 with saturated aqueous NaHCO₃ then extracted with EA (3×5 mL). The combined organics were washed with brine (1×10 mL), dried over anhydrous Na₂SO₄ and concentrated under reduced pressure. The residue was purified by prep-HPLC with the following conditions: Column, welch Vltimate XB—C18, 50×250 mm, 10 μm; Mobile Phase: 50-100% MeCN/0.1% aqueous formic acid to afford N-[2-cyano-4-fluoro-3-[([3-methyl-1H-pyrazolo[3,4-b]pyridin-5-yl]oxy)methyl]phenyl]-5-fluoro-2-methoxypyridine-3-sulfonamide (130 mg, 31% yield) as a light yellow solid.

LCMS (ES, m/z): [M+H]⁺: 487
¹H NMR (300 MHz, DMSO-d₆) δ 13.13 (s, 1H), 10.89 (s, 1H), 8.50 (d, J=2.9 Hz, 1H), 8.24 (d, J=2.8 Hz, 1H), 8.01 (dd, J=7.3, 3.0 Hz, 1H), 7.93 (d, J=2.8 Hz, 1H), 7.67 (t, J=9.1 Hz, 1H), 7.41 (dd, J=9.1, 4.8 Hz, 1H), 5.28-5.22 (m, 2H), 3.87 (s, 3H), 2.49 (s, 3H).

### Example 74: Synthesis of 5-fluoro-N-[3-fluoro-2-[([3-methyl-1H-pyrazolo[3,4-b]pyridin-5-yl]oxy)methyl]pyridin-4-yl]-2-methoxypyridine-3-sulfonamide (Compound 63)

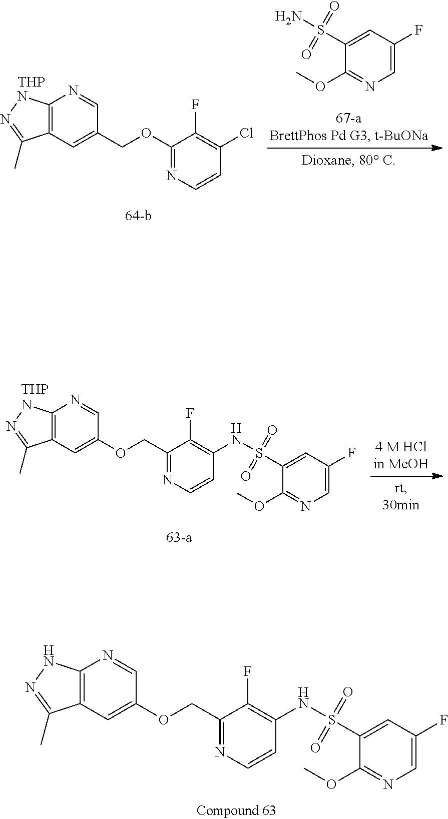 [SMILES: COc1ncc(F)cc1S(N)(=O)=O ; Cc1n[nH]c2ncc(COc3nccc(Cl)c3F)cc12 ; COc1ncc(F)cc1S(=O)(=O)Nc1ccnc(COc2cnc3[nH]nc(C)c3c2)c1F ; COc1ncc(F)cc1S(=O)(=O)Nc1ccnc(COc2cnc3[nH]nc(C)c3c2)c1F ; "THP" ; "THP"]

### Synthesis of 63-a: 5-fluoro-N-[3-fluoro-2-([[3-methyl-1-(oxan-2-yl)pyrazolo[3,4-b]pyridin-5-yl]oxy]methyl)pyridin-4-yl]-2-methoxypyridine-3-sulfonamide

Into a 40 mL vial was placed 4-chloro-3-fluoro-2-([[3-methyl-1-(oxan-2-yl)pyrazolo[3,4-b]pyridin-5-yl]oxy]methyl)pyridine (500 mg, 1.3 mmol, 1 equiv), dioxane (15 mL), 5-fluoro-2-methoxypyridine-3-sulfonamide (274 mg, 1.3 mmol, 1 equiv), t-BuONa (255 mg, 2.6 mmol, 2 equiv) and BrettPhos Pd G3 (481 mg, 0.5 mmol, 0.4 equiv) at room temperature under nitrogen atmosphere. The reaction was stirred for 2 h at 80° C. then concentrated. The residue was purified by Flash-Prep-HPLC with the following conditions: Column, WelFlash TM C18-I, Spherical C18 20-40 μm, 120 g; mobile phase: 20-70% MeCN/0.1% aqueous formic acid; to afford 5-fluoro-N-[3-fluoro-2-([[3-methyl-1-(oxan-2-yl)pyrazolo[3,4-b]pyridin-5-yl]oxy]methyl)pyridin-4-yl]-2-methoxypyridine-3-sulfonamide (400 mg, 55% yield) as a light yellow solid.

LCMS (ES, m/z): [M+H]⁺: 547

### Synthesis of Compound 63: 5-fluoro-N-[3-fluoro-2-[([3-methyl-1H-pyrazolo[3,4-b]pyridin-5-yl]oxy)methyl]pyridin-4-yl]-2-methoxypyridine-3-sulfonamide

Into a 40 mL vial was placed 5-fluoro-N-[3-fluoro-2-([[3-methyl-1-(oxan-2-yl)pyrazolo[3,4-b]pyridin-5-yl]oxy]methyl)pyridin-4-yl]-2-methoxypyridine-3-sulfonamide (100 mg, 0.2 mmol, 1 equiv) and 4 M HCl solution in MeOH (5 mL) at room temperature. The resulting solution was stirred for 1 h then concentrated. The residue was purified by prep-HPLC with the following conditions: Column, welch Vltimate XB—C18, 50×250 mm, 10 μm; Mobile Phase: 5-45% MeCN/0.1% aqueous formic acid; to afford 5-fluoro-N-[3-fluoro-2-[([3-methyl-1H-pyrazolo[3,4-b]pyridin-5-yl]oxy)methyl]pyridin-4-yl]-2-methoxypyridine-3-sulfonamide (50 mg, 59%) as a white solid.

LCMS (ES, m/z): [M+H]⁺: 463
¹H NMR (300 MHz, DMSO-d₆) δ 13.12 (s, 1H), 8.39 (s, 1H), 8.31 (s, 1H), 8.13 (s, 2H), 7.91 (s, 1H), 7.56 (s, 1H), 5.32 (s, 2H), 3.81 (s, 3H), 2.47 (s, 3H).

### Example 75: Synthesis of 5-chloro-N-[3-fluoro-2-[([3-methyl-1H-pyrazolo[3,4-b]pyridin-5-yl]oxy)methyl]pyridin-4-yl]-2-methoxypyridine-3-sulfonamide (Compound 64)

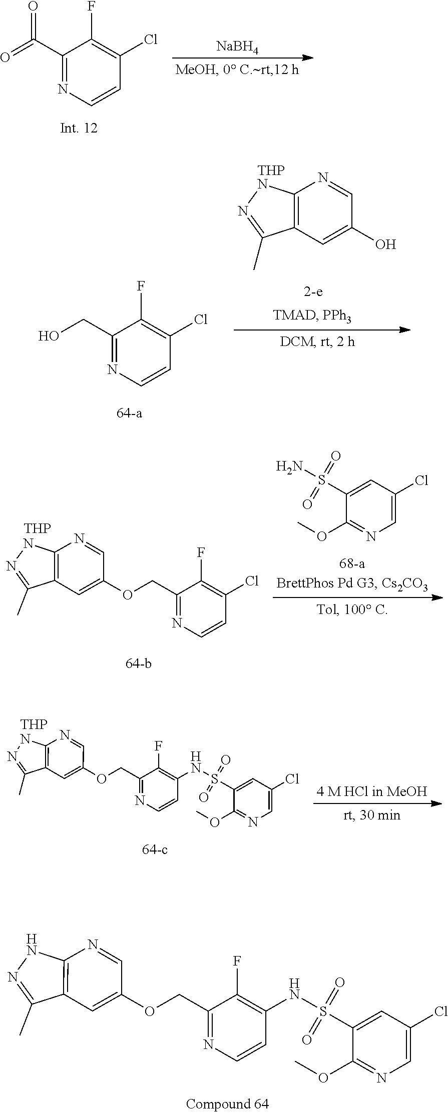

### Synthesis of 64-a: (4-chloro-3-fluoropyridin-2-yl)methanol

To a stirred solution of 4-chloro-3-fluoropyridine-2-carbaldehyde (10 g, 63 mmol, 1 equiv) in THE (150 mL) and MeOH (50 mL) was added NaBH₄ (6 g, 156 mmol, 2.5 equiv) in portions at 0° C. The resulting solution was stirred for 12 h at room temperature, then was quenched with water (10 mL) and extracted with ethyl acetate (4×50 mL). The combined organics were washed with brine (10 mL), dried over anhydrous sodium sulfate and concentrated. The residue was purified by silica gel column chromatography, eluting with PE:EA (5:1) to afford (4-chloro-3-fluoropyridin-2-yl)methanol (8 g, 79% yield) as light yellow oil.

LCMS (ES, m/z): [M+H]⁺: 162

### Synthesis of 64-b: 4-chloro-3-fluoro-2-([[3-methyl-1-(oxan-2-yl)pyrazolo[3,4-b]pyridin-5-yl]oxy]methyl)pyridine

Into a 40 mL vial was placed (4-chloro-3-fluoropyridin-2-yl)methanol (100 mg, 0.6 mmol, 1 equiv), DCM (6 mL), 3-methyl-1-(oxan-2-yl)pyrazolo[3,4-b]pyridin-5-ol (144 mg, 0.6 mmol, 1 equiv), PPh₃ (195 mg, 0.7 mmol, 1.2 equiv), and TMAD (128 mg, 0.7 mmol, 1.2 equiv) at room temperature. The reaction was stirred for 2 h, then concentrated. The residue was purified by Flash-Prep-HPLC with the following conditions: Column, WelFlash TM C18-I, Spherical C18 20-40 μm, 120 g; mobile phase: 30-80% MeCN/0.1% aqueous formic acid; to afford 4-chloro-3-fluoro-2-([[3-methyl-1-(oxan-2-yl)pyrazolo[3,4-b]pyridin-5-yl]oxy]methyl)pyridine (180 mg, 77% yield) as a white solid.

LCMS (ES, m/z): [M+H]⁺: 377

### Synthesis of 64-c: 5-chloro-N-[3-fluoro-2-([[3-methyl-1-(oxan-2-yl)pyrazolo[3,4-b]pyridin-5-yl]oxy]methyl)pyridin-4-yl]-2-methoxypyridine-3-sulfonamide

Into a 40 mL vial was placed 4-chloro-3-fluoro-2-([[3-methyl-1-(oxan-2-yl)pyrazolo[3,4-b]pyridin-5-yl]oxy]methyl)pyridine (500 mg, 1.3 mmol, 1 equiv), toluene (5 mL), 5-chloro-2-methoxypyridine-3-sulfonamide (591 mg, 2.6 mmol, 2 equiv), Cs₂CO₃ (865 mg, 2.6 mmol, 2 equiv) and BrettPhos Pd G3 (481 mg, 0.5 mmol, 0.4 equiv). The resulting solution was stirred for 12 h at 100° C. under nitrogen atmosphere. The reaction mixture was concentrated and the residue purified by Flash-Prep-HPLC with the following conditions: Column, WelFlash TM C18-I, Spherical C18 20-40 μm, 120 g; mobile phase: 30-80% MeCN/0.1% aqueous formic acid; to afford 5-chloro-N-[3-fluoro-2-([[3-methyl-1-(oxan-2-yl)pyrazolo[3,4-b]pyridin-5-yl]oxy]methyl)pyridin-4-yl]-2-methoxypyridine-3-sulfonamide (300 mg, 40% yield) as a light yellow solid.

LCMS (ES, m/z): [M+H]⁺: 563

### Synthesis of Compound 64: 5-chloro-N-[3-fluoro-2-[([3-methyl-1H-pyrazolo[3,4-b]pyridin-5-yl]oxy)methyl]pyridin-4-yl]-2-methoxypyridine-3-sulfonamide

Into a 40 mL vial was placed 5-chloro-N-[3-fluoro-2-([[3-methyl-1-(oxan-2-yl)pyrazolo[3,4-b]pyridin-5-yl]oxy]methyl)pyridin-4-yl]-2-methoxypyridine-3-sulfonamide (100 mg, 0.2 mmol, 1 equiv) and 4 M HCl solution in MeOH (5 mL) at room temperature. The resulting solution was stirred for 1 h, then was concentrated. The residue was purified by prep-HPLC with the following conditions: Column, welch Vltimate XB—C18, 50×250 mm, 10 μm; Mobile Phase: 10-45% MeCN/0.1 aqueous formic acid; to afford 5-chloro-N-[3-fluoro-2-[([3-methyl-1H-pyrazolo[3,4-b]pyridin-5-yl]oxy)methyl]pyridin-4-yl]-2-methoxypyridine-3-sulfonamide (50 mg, 59% yield) as a white solid.

LCMS (ES, m/z): [M+H]⁺: 479
¹H NMR (300 MHz, DMSO-d₆) δ 13.13 (s, 1H), 8.43 (s, 1H), 8.32 (s, 1H), 8.21 (s, 1H), 8.14 (s, 1H), 7.91 (s, 1H), 7.57 (s, 1H), 5.33 (s, 2H), 3.82 (s, 3H), 2.47 (s, 3H)

### Example 76: Synthesis of 5-chloro-N-[3,5-difluoro-4-[([3-methyl-1H-pyrazolo[3,4-b]pyridin-5-yl]oxy)methyl]pyridin-2-yl]-2-methoxypyridine-3-sulfonamide (Compound 65)

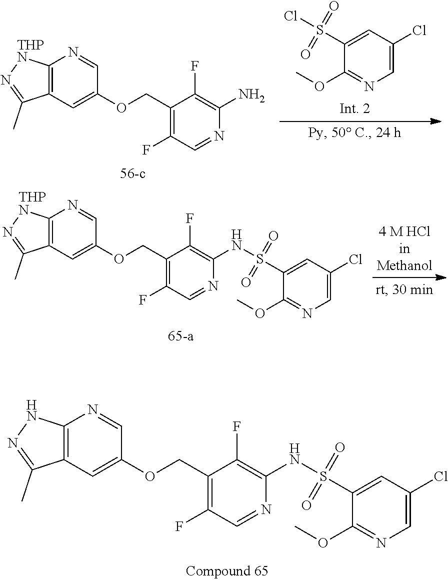

### Synthesis of 65-a: 5-chloro-N-[3,5-difluoro-4-([[3-methyl-1-(oxan-2-yl)pyrazolo[3,4-b]pyridin-5-yl]oxy]methyl)pyridin-2-yl]-2-methoxypyridine-3-sulfonamide

Into a 40 mL vial was placed 3,5-difluoro-4-([[3-methyl-1-(oxan-2-yl)pyrazolo[3,4-b]pyridin-5-yl]oxy]methyl)pyridin-2-amine (200 mg, 0.5 mmol, 1 equiv), pyridine (8 mL) and 5-chloro-2-methoxypyridine-3-sulfonyl chloride (387 mg, 1.5 mmol, 3 equiv). The resulting solution was stirred for 24 h at 50° C. then diluted with H₂O (50 mL) and extracted with ethyl acetate (3×20 mL). The combined organics were washed with brine (2×20 mL), dried over anhydrous sodium sulfate and concentrated. The residue was purified by Flash-Prep-HPLC with the following conditions: Column, WelFlash TM C18-I, Spherical C18 20-m, 120 g; mobile phase: 30-80% MeCN/0.1% aqueous formic acid; to afford 5-chloro-N-[3,5-difluoro-4-([[3-methyl-1-(oxan-2-yl)pyrazolo[3,4-b]pyridin-5-yl]oxy]methyl)pyridin-2-yl]-2-methoxypyridine-3-sulfonamide (157 mg, 51% yield) as a white solid.

LCMS (ES, m/z): [M+H]⁺: 581

### Synthesis of Compound 65: 5-chloro-N-[3,5-difluoro-4-[([3-methyl-1H-pyrazolo[3,4-b]pyridin-5-yl]oxy)methyl]pyridin-2-yl]-2-methoxypyridine-3-sulfonamide

Into a 40 mL vial was placed 5-chloro-N-[3,5-difluoro-4-([[3-methyl-1-(oxan-2-yl)pyrazolo[3,4-b]pyridin-5-yl]oxy]methyl)pyridin-2-yl]-2-methoxypyridine-3-sulfonamide (100 mg, 0.2 mmol, 1 equiv) and 4 M HCl solution in MeOH (5 mL). The resulting solution was stirred for 30 min then concentrated. The residue was purified by prep-HPLC with the following conditions: Column, welch Vltimate XB—C18, 50×250 mm, 10 μm; Mobile Phase: 30-60% MeCN/0.1% aqueous formic acid; to afford 5-chloro-N-[3,5-difluoro-4-[([3-methyl-1H-pyrazolo[3,4-b]pyridin-5-yl]oxy)methyl]pyridin-2-yl]-2-methoxypyridine-3-sulfonamide (50 mg, 58% yield) as a white solid.

LCMS (ES, m/z): [M+H]⁺: 497
¹H NMR (300 MHz, DMSO-d₆) δ 13.13 (s, 1H), 11.46 (s, 1H), 8.52 (d, J=2.6 Hz, 1H), 8.29-8.18 (m, 3H), 7.91 (d, J=2.8 Hz, 1H), 5.29 (s, 2H), 3.92 (s, 3H), 2.48 (s, 3H).

### Example 77: Synthesis of N-[3-chloro-5-fluoro-4-[([3-methyl-1H-pyrazolo[3,4-b]pyridin-5-yl]oxy)methyl]pyridin-2-yl]-5-fluoro-2-methoxypyridine-3-sulfonamide (Compound 66)

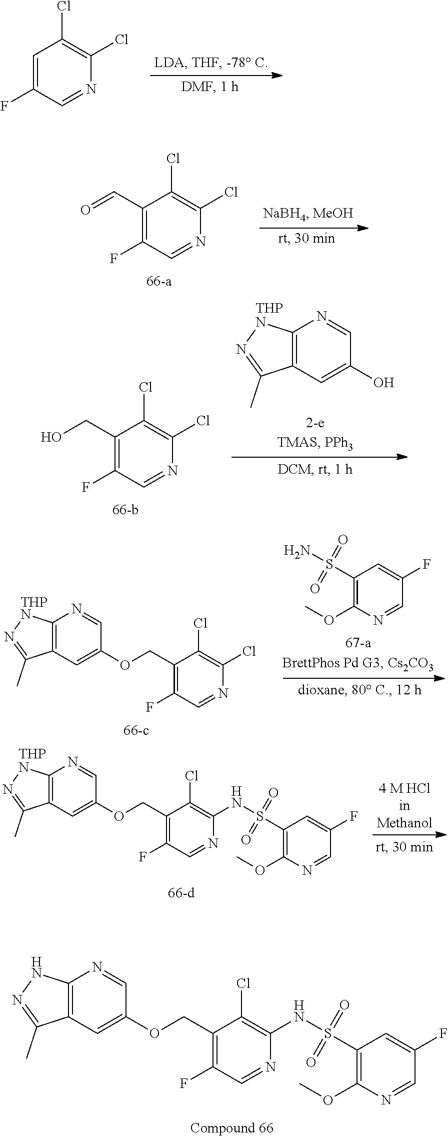

### Synthesis of 66-a: 2,3-dichloro-5-fluoropyridine-4-carbaldehyde

To a stirred solution of 2,3-dichloro-5-fluoropyridine (1 g, 6 mmol, 1 equiv) in THF (20 mL) was added 2 M LDA in THF (4.5 mL, 9 mmol, 1.5 equiv) dropwise at −78° C. The resulting solution was stirred for 1 h at −78° C., then DMF (881 mg, 12 mmol, 2 equiv) was added dropwise at −78° C. and the reaction stirred for 1 h at this temperature. The reaction was quenched with 1M aq. HCl (10 mL) at −5° C. and extracted with EA (3×50 mL). The combined organics were washed with brine (2×20 mL), dried over anhydrous sodium sulfate and concentrated to afford 2,3-dichloro-5-fluoropyridine-4-carbaldehyde (1 g, crude) as a yellow oil which was used in the next step directly without further purification.

### Synthesis of 66-b: (2,3-dichloro-5-fluoropyridin-4-yl)methanol

To a stirred solution of 2,3-dichloro-5-fluoropyridine-4-carbaldehyde (1 g, 5 mmol, 1 equiv) in MeOH (20 mL) was added NaBH₄ (0.6 g, 15 mmol, 3 equiv) in portions at 0° C. The reaction was stirred for 30 min at room temperature, then was quenched with saturated aqueous NH₄Cl (50 mL). The resulting mixture was extracted with EA (3×100 mL), and the combined organic layers were washed with brine (3×50 mL), dried over anhydrous Na₂SO₄ and concentrated. The residue was purified by silica gel column chromatography, eluting with PE:EA (2:1) to afford (2,3-dichloro-5-fluoropyridin-4-yl)methanol (300 mg, 30% yield) as a yellow oil.

LCMS (ES, m/z): [M+H]⁺: 196

### Synthesis of 66-c: 2,3-dichloro-5-fluoro-4-([[3-methyl-1-(oxan-2-yl)pyrazolo[3,4-b]pyridin-5-yl]oxymethyl)pyridine

To a stirred solution of (2,3-dichloro-5-fluoropyridin-4-yl)methanol (300 mg, 1.5 mmol, 1 equiv) in DCM (10 mL) were added 3-methyl-1-(oxan-2-yl)pyrazolo[3,4-b]pyridin-5-ol (393 mg, 1.7 mmol, 1.1 equiv), PPh₃ (602 mg, 2.3 mmol, 1.5 equiv), and TMAD (395 mg, 2.3 mmol, 1.5 equiv). The resulting mixture was stirred for 1 h, then concentrated under vacuum. The residue was purified by Flash-Prep-HPLC with the following conditions: Column, WelFlash TM C18-I, Spherical C18 20-40 μm, 120 g; mobile phase: 30-80% MeCN/0.1% aqueous formic acid; to afford 2,3-dichloro-5-fluoro-4-([[3-methyl-1-(oxan-2-yl)pyrazolo[3,4-b]pyridin-5-yl]oxy]methyl)pyridine (490 mg, 78% yield) as a white solid.

LCMS (ES, m/z): [M+H]⁺: 411

### Synthesis of 66-d: N-[3-chloro-5-fluoro-4-([[3-methyl-1-(oxan-2-yl)pyrazolo[3,4-b]pyridin-5-yl]oxymethyl)pyridin-2-yl]-5-fluoro-2-methoxypyridine-3-sulfonamide

Into a 40 mL vial was placed 2,3-dichloro-5-fluoro-4-([[3-methyl-1-(oxan-2-yl)pyrazolo[3,4-b]pyridin-5-yl]oxy]methyl)pyridine (800 mg, 2 mmol, 1 equiv), dioxane (9 mL), 5-fluoro-2-methoxypyridine-3-sulfonamide (602 mg, 3 mmol, 1.5 equiv), Cs₂CO₃ (1.3 g, 4 mmol, 2 equiv) and BrettPhos Pd G3 (705 mg, 0.8 mmol, 0.4 equiv) at room temperature under nitrogen atmosphere. The reaction was stirred for 12 h at 80° C. then diluted with H₂O (20 mL) and extracted with ethyl acetate (3×10 mL). The combined organics were washed with brine (10 mL), dried over anhydrous sodium sulfate and concentrated. The residue was purified by Flash-Prep-HPLC with the following conditions: Column, WelFlash TM C18-I, Spherical C18 20-40 μm, 120 g; mobile phase: 30-80% MeCN/0.1% aqueous formic acid; to afford N-[3-chloro-5-fluoro-4-([[3-methyl-1-(oxan-2-yl)pyrazolo[3,4-b]pyridin-5-yl]oxy]methyl)pyridin-2-yl]-5-fluoro-2-methoxypyridine-3-sulfonamide (100 mg, 9% yield) as a light yellow solid.

LCMS (ES, m/z): [M+H]⁺: 581

### Synthesis of Compound 66: N-[3-chloro-5-fluoro-4-[([3-methyl-1H-pyrazolo[3,4-b]pyridin-5-yl]oxy)methyl]pyridin-2-yl]-5-fluoro-2-methoxypyridine-3-sulfonamide

Into a 40 mL vial was placed N-[3-chloro-5-fluoro-4-([[3-methyl-1-(oxan-2-yl)pyrazolo[3,4-b]pyridin-5-yl]oxy]methyl)pyridin-2-yl]-5-fluoro-2-methoxypyridine-3-sulfonamide (100 mg, 0.2 mmol, 1 equiv) and 4 M HCl in MeOH (5 mL). The resulting solution was stirred for 30 min at room temperature, then concentrated. The residue was purified by prep-HPLC with the following conditions: Column, welch Vltimate XB—C18, 50×250 mm, 10 μm; Mobile Phase: 20-60% MeCN/0.1% aqueous formic acid; to afford N-[3-chloro-5-fluoro-4-[([3-methyl-1H-pyrazolo[3,4-b]pyridin-5-yl]oxy)methyl]pyridin-2-yl]-5-fluoro-2-methoxypyridine-3-sulfonamide (50 mg, 58% yield) as a white solid.

LCMS (ES, m/z): [M+H]⁺: 497
¹H NMR (300 MHz, DMSO-d₆) δ 13.13 (s, 1H), 11.17 (s, 1H), 8.48 (d, J=2.9 Hz, 1H), 8.35-8.23 (m, 2H), 8.12 (dd, J=7.5, 3.0 Hz, 1H), 7.93 (d, J=2.7 Hz, 1H), 5.28 (d, J=1.7 Hz, 2H), 3.93 (s, 3H), 2.49 (s, 3H).

### Example 78: Synthesis of 5-fluoro-N-[5-fluoro-6-[([3-methyl-1H-pyrazolo[3,4-b]pyridin-5-yl]oxy)methyl]pyridine-2-yl]-2-methoxypyridine-3-sulfonamide (Compound 67)

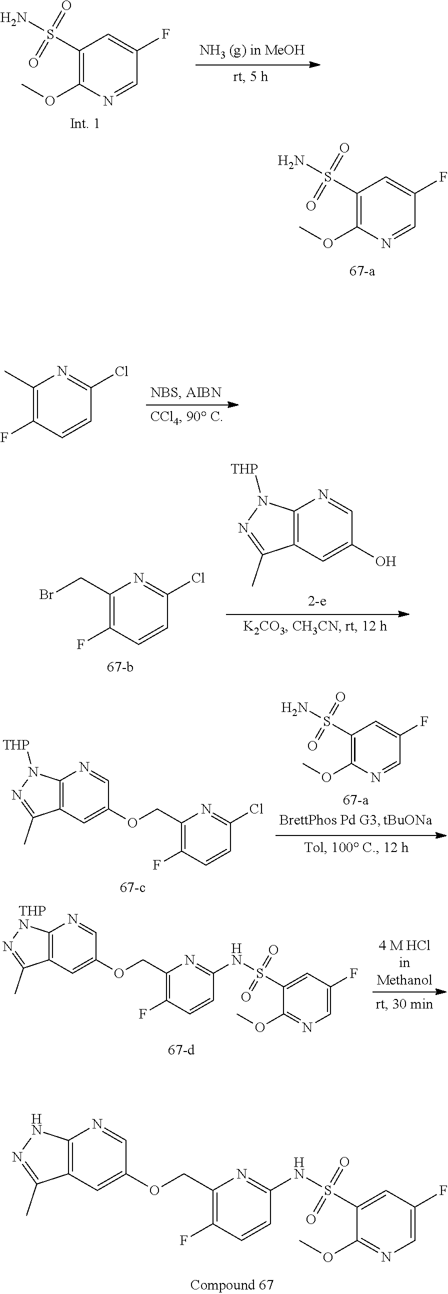

### Synthesis of 67-a: 5-fluoro-2-methoxypyridine-3-sulfonamide

A mixture of 5-fluoro-2-methoxypyridine-3-sulfonyl chloride (2.2 g, 9.8 mmol, 1 equiv) and 7 M NH₃ solution in MeOH (30 mL) was stirred for 5 h at room temperature. The resulting mixture was concentrated under reduced pressure. The residue was purified by silica gel column chromatography, eluting with PE:EA (2:1) to give 5-fluoro-2-methoxypyridine-3-sulfonamide (1.1 g, 55% yield) as a white solid.

LCMS (ES, m/z): [M+H]⁺: 207

### Synthesis of 67-b: 2-(bromomethyl)-6-chloro-3-fluoropyridine

To a stirred solution of 6-chloro-3-fluoro-2-methylpyridine (5 g, 34 mmol, 1 equiv) and NBS (7.4 g, 41 mmol, 1.2 equiv) in CCl₄ (50 mL) was added AIBN (1 g, 6.9 mmol, 0.2 equiv) in portions at room temperature. The reaction was stirred overnight at 90° C., then cooled and diluted with water (300 mL). The resulting solution was extracted with EA (3×100 mL), and the combined organics were washed with brine (50 mL), dried over anhydrous sodium sulfate and concentrated. The residue was purified by silica gel column chromatography, eluting with PE:EA (20:1) to afford 2-(bromomethyl)-6-chloro-3-fluoropyridine (3.6 g, 47% yield) as a yellow solid.

LCMS (ES, m/z): [M+H]⁺: 224

### Synthesis of 67-c: 6-chloro-3-fluoro-2-([[3-methyl-1-(oxan-2-yl)pyrazolo[3,4-b]pyridin-5-yl]oxy]methyl)pyridine

To a stirred solution of 2-(bromomethyl)-6-chloro-3-fluoropyridine (100 mg, 0.5 mmol, 1 equiv) in MeCN (5 mL) were added 3-methyl-1-(oxan-2-yl)pyrazolo[3,4-b]pyridin-5-ol (114 mg, 0.5 mmol, 1 equiv) and K₂CO₃ (123 mg, 0.9 mmol, 1.2 equiv). The reaction was stirred overnight then diluted with water (20 mL) and extracted with EA (3×20 mL). The combined organics were washed with brine (10 mL), dried over anhydrous sodium sulfate and concentrated. The residue was purified by silica gel column chromatography, eluting with PE:EA (1:1) to afford 6-chloro-3-fluoro-2-([[3-methyl-1-(oxan-2-yl)pyrazolo[3,4-b]pyridin-5-yl]oxy]methyl)pyridine (100 mg, 60% yield) as a yellow solid.

LCMS (ES, m/z): [M+H]⁺: 377

### Synthesis of 67-d: 5-fluoro-N-[5-fluoro-6-([[3-methyl-1-(oxan-2-yl)pyrazolo[3,4-b]pyridin-5-yl]oxy]methyl)pyridin-2-yl]-2-methoxypyridine-3-sulfonamide

Into a 40 mL vial was placed 6-chloro-3-fluoro-2-([[3-methyl-1-(oxan-2-yl)pyrazolo[3,4-b]pyridin-5-yl]oxy]methyl)pyridine (500 mg, 1.3 mmol, 1 equiv), toluene (15 mL), 5-fluoro-2-methoxypyridine-3-sulfonamide (411 mg, 2 mmol, 1.5 equiv), Cs₂CO₃ (865 mg, 2.7 mmol, 2 equiv) and BrettPhos Pd G3 (482 mg, 0.5 mmol, 0.4 equiv). The reaction was stirred for 12 h at 100° C. under nitrogen atmosphere, then concentrated. The residue was purified by Flash-Prep-HPLC with the following conditions: Column, WelFlash TM C18-I, Spherical C18 20-40 μm, 120 g; mobile phase: 30-80% MeCN/0.1% aqueous formic acid; to afford 5-fluoro-N-[5-fluoro-6-([[3-methyl-1-(oxan-2-yl)pyrazolo[3,4-b]pyridin-5-yl]oxy]methyl)pyridin-2-yl]-2-methoxypyridine-3-sulfonamide (180 mg, 25% yield) as a white solid.

LCMS (ES, m/z): [M+H]⁺: 547

### Synthesis of Compound 67: 5-fluoro-N-[5-fluoro-6-[([3-methyl-1H-pyrazolo[3,4-b]pyridin-5-yl]oxy)methyl]pyridine-2-yl]-2-methoxypyridine-3-sulfonamide

Into a 40 mL vial was placed 5-fluoro-N-[5-fluoro-6-([[3-methyl-1-(oxan-2-yl)pyrazolo[3,4-b]pyridin-5-yl]oxy]methyl)pyridin-2-yl]-2-methoxypyridine-3-sulfonamide (100 mg, 0.2 mmol, 1 equiv) and 4 M HCl solution in MeOH (5 mL). The resulting solution was stirred for 30 min then concentrated. The residue was purified by prep-HPLC with the following conditions: Column, welch Vltimate XB—C18, 50×250 mm, 10 μm; Mobile Phase: 20-55% MeCN/0.1% aqueous formic acid; to afford 5-fluoro-N-[5-fluoro-6-[([3-methyl-1H-pyrazolo[3,4-b]pyridin-5-yl]oxy)methyl]pyridine-2-yl]-2-methoxypyridine-3-sulfonamide (50 mg, 59% yield) as a white solid.

LCMS (ES, m/z): [M+H]⁺: 463
¹H NMR (300 MHz, DMSO-d₆) δ 13.07 (s, 1H), 11.44 (s, 1H), 8.37 (d, J=3.0 Hz, 1H), 8.16 (dt, J=5.2, 2.8 Hz, 2H), 7.80-7.68 (m, 2H), 7.10 (dd, J=8.9, 3.2 Hz, 1H), 5.06 (d, J=2.1 Hz, 2H), 3.84 (s, 3H), 2.46 (s, 3H).

### Example 79: Synthesis of 5-chloro-N-[5-fluoro-6-[([3-methyl-1H-pyrazolo[3,4-b]pyridin-5-yl]oxy)methyl]pyridin-2-yl]-2-methoxypyridine-3-sulfonamide (Compound 68)

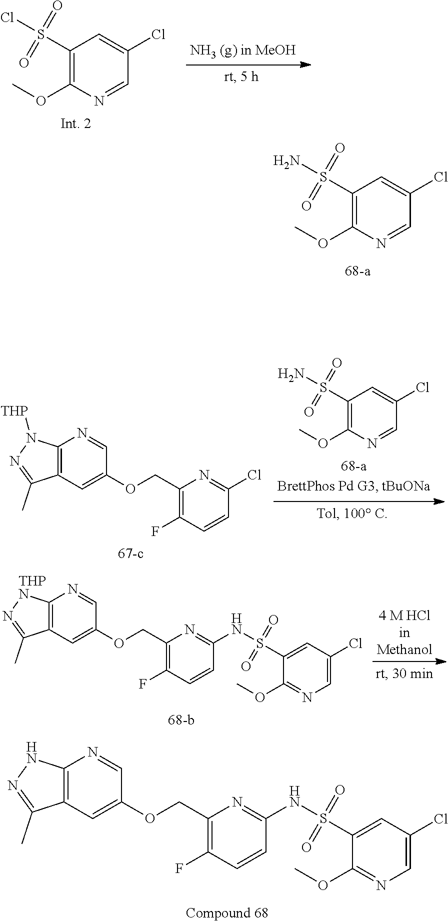

### Synthesis of 68-a: 5-chloro-2-methoxypyridine-3-sulfonamide

A solution of 5-chloro-2-methoxypyridine-3-sulfonyl chloride (2 g, 8.3 mmol, 1 equiv) and 7 M NH₃ solution in MeOH (40 mL) was stirred for 5 h at room temperature. The reaction mixture was concentrated and the residue purified by silica gel column chromatography, eluting with PE:EA (2:1) to afford 5-chloro-2-methoxypyridine-3-sulfonamide (1.7 g, 92% yield) as a white solid.

LCMS (ES, m/z): [M+H]⁺: 223

### Synthesis of 68-b: 5-chloro-N-[5-fluoro-6-([[3-methyl-1-(oxan-2-yl)pyrazolo[3,4-b]pyridin-5-yl]oxy]methyl)pyridin-2-yl]-2-methoxypyridine-3-sulfonamide

Into a 40 mL vial was placed 6-chloro-3-fluoro-2-([[3-methyl-1-(oxan-2-yl)pyrazolo[3,4-b]pyridin-5-yl]oxy]methyl)pyridine (800 mg, 2 mmol, 1 equiv), toluene (24 mL), 5-chloro-2-methoxypyridine-3-sulfonamide (709 mg, 3 mmol, 1.5 equiv), Cs₂CO₃ (1.4 mg, 4 mmol, 2 equiv) and BrettPhos Pd G3 (770 mg, 0.9 mmol, 0.4 equiv). The reaction was stirred for 12 h at 100° C. under nitrogen atmosphere, then concentrated. The residue was purified by Flash-Prep-HPLC with the following conditions: Column, WelFlash TM C18-I, Spherical C18 20-40 μm, 120 g; mobile phase: 30-80% MeCN/0.1% aqueous formic acid; to afford 5-chloro-N-[5-fluoro-6-([[3-methyl-1-(oxan-2-yl)pyrazolo[3,4-b]pyridin-5-yl]oxy]methyl)pyridin-2-yl]-2-methoxypyridine-3-sulfonamide (560 mg, 47% yield) as a yellow solid.

LCMS (ES, m/z): [M+H]⁺: 563

### Synthesis of Compound 68: 5-chloro-N-[5-fluoro-6-[([3-methyl-1H-pyrazolo[3,4-b]pyridin-5-yl]oxy)methyl]pyridin-2-yl]-2-methoxypyridine-3-sulfonamide

Into a 40 mL round-bottom flask was placed 5-chloro-N-[5-fluoro-6-([[3-methyl-1-(oxan-2-yl)pyrazolo[3,4-b]pyridin-5-yl]oxy]methyl)pyridin-2-yl]-2-methoxypyridine-3-sulfonamide (100 mg, 0.2 mmol, 1 equiv) and 4 M HCl solution in MeOH (5 mL). The resulting solution was stirred for 30 min then concentrated. The residue was purified by prep-HPLC with the following conditions: Column, welch Vltimate XB—C18, 50×250 mm, 10 μm; Mobile Phase: 20-60% MeCN/0.1% aqueous formic acid; to afford 5-chloro-N-[5-fluoro-6-[([3-methyl-1H-pyrazolo[3,4-b]pyridin-5-yl]oxy)methyl]pyridin-2-yl]-2-methoxypyridine-3-sulfonamide (50 mg, 59% yield) as a white solid.

LCMS (ES, m/z): [M+H]⁺: 479
¹H NMR (300 MHz, DMSO-d₆) δ 13.07 (s, 1H), 11.46 (s, 1H), 8.40 (d, J=2.6 Hz, 1H), 8.26 (d, J=2.7 Hz, 1H), 8.18 (d, J=2.7 Hz, 1H), 7.80-7.68 (m, 2H), 7.08 (dd, J=9.0, 3.2 Hz, 1H), 5.04 (d, J=2.0 Hz, 2H), 3.85 (s, 3H), 2.46 (s, 3H).

### Example 80: Synthesis of N-[3-[([3-ethynyl-1H-Pyrazolo[3,4-b]pyridin-5-yl]oxy)methyl]-2,4-difluorophenyl]-5-fluoro-2-methoxypyridine-3-sulfonamide (Compound 69)

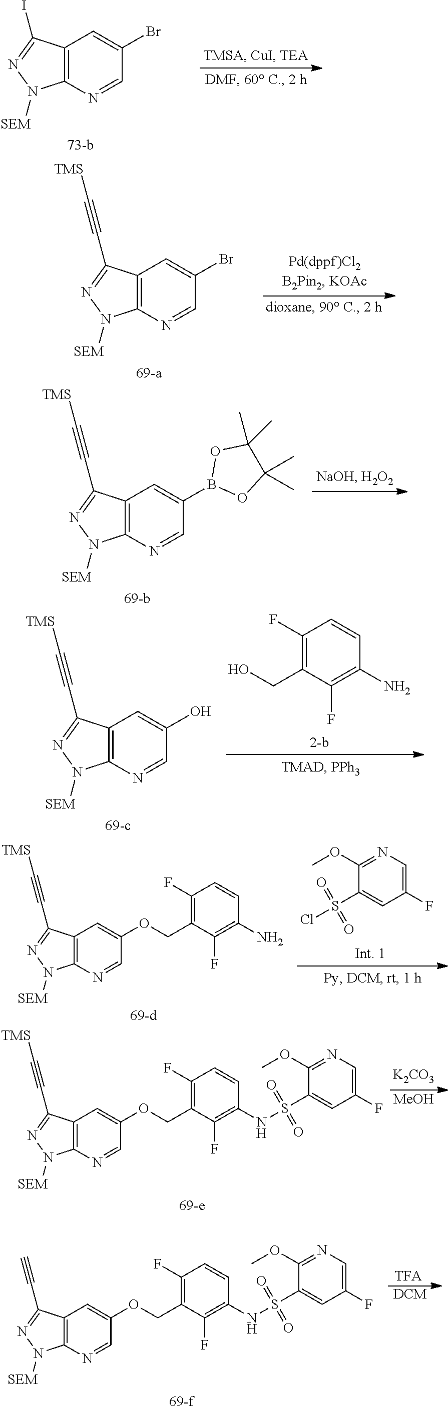

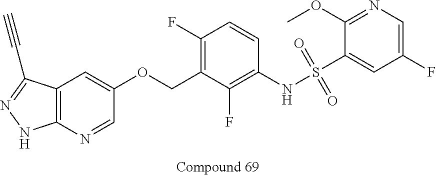 [SMILES: C#Cc1n[nH]c2ncc(OCc3c(F)ccc(NS(=O)(=O)c4cc(F)cnc4OC)c3F)cc12]

### Synthesis of 69-a: 5-bromo-1-[[2-(trimethylsilyl)ethoxy]methyl]-3-[2-(trimethylsilyl)ethynyl]pyrazolo[3,4-b]pyridine

To a stirred solution of 5-bromo-3-iodo-1-[[2-(trimethylsilyl)ethoxy]methyl]pyrazolo[3,4-b]pyridine (800 mg, 1.7 mmol, 1 equiv) and trimethylsilylacetylene (190 mg, 1.9 mmol, 1.1 equiv) in DMF (10 mL) were added TEA (534 mg, 5.2 mmol, 3 equiv), CuI (33 mg, 0.17 mmol, 0.1 equiv) and Pd(PPh₃)₂Cl₂ (123 mg, 0.17 mmol, 0.1 equiv) at room temperature under nitrogen atmosphere. The reaction was stirred for 2 h at 60° C. under nitrogen atmosphere, then cooled and quenched with water (50 mL). The resulting mixture was extracted with EA (3×100 mL), and the combined organics were washed with brine (3×50 mL), dried over anhydrous Na₂SO₄ and concentrated. The residue was purified by silica gel column chromatography, eluting with PE:EA (20:1) to afford 5-bromo-1-[[2-(trimethylsilyl)ethoxy]methyl]-3-[2-(trimethylsilyl)ethynyl]pyrazolo[3,4-b]pyridine (700 mg, 94% yield) as a colorless oil.

LCMS (ES, m/z): [M+H]⁺: 424

### Synthesis of 69-b/c: 1-((2-(trimethylsilyl)ethoxy)methyl)-3-((trimethylsilyl)ethynyl)-1H-pyrazolo[3,4-b]pyridin-5-ol

To a solution of 5-bromo-1-[[2-(trimethylsilyl)ethoxy]methyl]-3-[2-(trimethylsilyl)ethynyl]pyrazolo[3,4-b]pyridine (700 mg, 1.6 mmol, 1 equiv) and bis(pinacolato)diboron (837 mg, 3.2 mmol, 2 equiv) in dioxane (20 mL) were added KOAc (323 mg, 3.2 mmol, 2 equiv) and Pd(dppf)Cl₂ (120 mg, 0.16 mmol, 0.1 equiv) at room temperature under nitrogen atmosphere. After stirring for 2 h at 90° C., the resulting mixture was allowed to cool and was diluted with THE (10 mL). 2% aqueous NaOH (10 mL) was added, followed by 30% H₂O₂ (1.92 g, 16.9 mmol, 10 equiv) dropwise at room temperature. The reaction was stirred for 1 h, then diluted with water (50 mL) and extracted with EA (3×20 mL). The combined organics were washed with brine (3×50 mL), dried over anhydrous Na₂SO₄ and concentrated. The residue was purified by silica gel column chromatography, eluting with PE:EA (2:1) to afford 1-[[2-(trimethylsilyl)ethoxy]methyl]-3-[2-(trimethylsilyl)ethynyl]pyrazolo[3,4-b]pyridin-5-ol (500 mg, 84% yield) as a yellow oil.

LCMS (ES, m/z): [M+H]⁺: 362

### Synthesis of 69-d: 2,4-difluoro-3-[[(1-[[2-(trimethylsilyl)ethoxy]methyl]-3-[2-(trimethylsilyl)ethynyl]pyrazolo[3,4-b]pyridin-5-yl)oxy]methyl]aniline

To a stirred solution of 1-[[2-(trimethylsilyl)ethoxy]methyl]-3-[2-(trimethylsilyl)ethynyl]pyrazolo[3,4-b]pyridin-5-ol (500 mg, 1.4 mmol, 1 equiv) and (3-amino-2,6-difluorophenyl)methanol (220 mg, 1.4 mmol, 1 equiv) in DCM (10 mL) were added TMAD (357 mg, 2.1 mmol, 1.5 equiv) and PPh₃ (544 mg, 2.1 mmol, 1.5 equiv) at room temperature. The resulting mixture was stirred for 1 h, then concentrated. The residue was diluted with water (50 mL) and extracted with EA (3×100 mL). The combined organics were washed with brine (3×50 mL), dried over anhydrous Na₂SO₄ and concentrated. The residue was purified by silica gel column chromatography, eluting with PE:EA (3:1) to afford 2,4-difluoro-3-[[(1-[[2-(trimethylsilyl)ethoxy]methyl]-3-[2-(trimethylsilyl)ethynyl]pyrazolo[3,4-b]pyridin-5-yl)oxy]methyl]aniline (300 mg, 43% yield) as a yellow oil.

LCMS (ES, m/z): [M+H]⁺: 503

### Synthesis of 69-e: N-(2,4-difluoro-3-[[(1-[[2-(trimethylsilyl)ethoxy]methyl]-3-[2-(trimethylsilyl)ethynyl]pyrazolo[3,4-b]pyridin-5-yl)oxy]methyl]phenyl)-5-fluoro-2-methoxypyridine-3-sulfonamide

To a stirred solution of 2,4-difluoro-3-[[(1-[[2-(trimethylsilyl)ethoxy]methyl]-3-[2-(trimethylsilyl)ethynyl]pyrazolo[3,4-b]pyridin-5-yl)oxy]methyl]aniline (280 mg, 0.55 mmol, 1 equiv) and pyridine (132 mg, 1.6 mmol, 3 equiv) in DCM (5 mL) was added 5-fluoro-2-methoxypyridine-3-sulfonyl chloride (188 mg, 0.8 mmol, 1.5 equiv) in portions. The reaction was stirred for 1 h then concentrated. The residue was purified by silica gel column chromatography, eluting with PE:EA (1:1) to afford N-(2,4-difluoro-3-[[(1-[[2-(trimethylsilyl)ethoxy]methyl]-3-[2-(trimethylsilyl)ethynyl]pyrazolo[3,4-b]pyridin-5-yl)oxy]methyl]phenyl)-5-fluoro-2-methoxypyridine-3-sulfonamide (200 mg, 52% yield) as a yellow solid.

LCMS (ES, m/z): [M+H]⁺: 692

### Synthesis of 69-f: N-(3-[[(3-ethynyl-1-[[2-(trimethylsilyl)ethoxy]methyl]pyrazolo[3,4-b]pyridin-5-yl)oxy]methyl]-2,4-difluorophenyl)-5-fluoro-2-methoxypyridine-3-sulfonamide

To a stirred solution of N-(2,4-difluoro-3-[[(1-[[2-(trimethylsilyl)ethoxy]methyl]-3-[2-(trimethylsilyl)ethynyl]pyrazolo[3,4-b]pyridin-5-yl)oxy]methyl]phenyl)-5-fluoro-2-methoxypyridine-3-sulfonamide (200 mg, 0.29 mmol, 1 equiv) in MeOH (5 mL) was added K₂CO₃ (111 mg, 0.87 mmol, 3 equiv). The resulting mixture was stirred for 6 h at room temperature, then concentrated. The residue was purified by silica gel column chromatography, eluting with PE:EA (2:1) to afford N-(3-[[(3-ethynyl-1-[[2-(trimethylsilyl)ethoxy]methyl]pyrazolo[3,4-b]pyridin-5-yl)oxy]methyl]-2,4-difluorophenyl)-5-fluoro-2-methoxypyridine-3-sulfonamide (150 mg, 84% yield) as a yellow solid.

LCMS (ES, m/z): [M+H]⁺: 620

### Synthesis of Compound 69: N-[3-[([3-ethynyl-1H-pyrazolo[3,4-b]pyridin-5-yl]oxy)methyl]-2,4-difluorophenyl]-5-fluoro-2-methoxypyridine-3-sulfonamide

To a stirred solution of N-(3-[[(3-ethynyl-1-[[2-(trimethylsilyl)ethoxy]methyl]pyrazolo[3,4-b]pyridin-5-yl)oxy]methyl]-2,4-difluorophenyl)-5-fluoro-2-methoxypyridine-3-sulfonamide (120 mg, 0.19 mmol, 1 equiv) in DCM (4 mL) was added TFA (1 mL). The resulting mixture was stirred for 2 h then concentrated. The residue was purified by prep-HPLC with following conditions: Column, welch Vltimate XB—C18, 50×250 mm, 10 m; Mobile Phase: 25-60% MeCN/0.1% aqueous formic acid; to afford N-[3-[([3-ethynyl-1H-pyrazolo[3,4-b]pyridin-5-yl]oxy)methyl]-2,4-difluorophenyl]-5-fluoro-2-methoxypyridine-3-sulfonamide (60 mg, 42% yield) as a white solid.

LCMS (ES, m/z): [M+H]⁺: 490
¹H NMR (300 MHz, DMSO-d₆) δ 13.97 (s, 1H), 10.37 (s, 1H), 8.44 (d, J=3.0 Hz, 1H), 8.28 (d, J=2.7 Hz, 1H), 7.98 (dd, J=7.3, 3.0 Hz, 1H), 7.81 (d, J=2.7 Hz, 1H), 7.39 (td, J=8.9, 5.9 Hz, 1H), 7.17 (td, J=9.0, 1.6 Hz, 1H), 5.20 (s, 2H), 4.57 (s, 1H), 3.90 (3H, s).

### Example 81: Synthesis of N-(3-(((3-cyclopropyl-11H-Pyrazolo[3,4-b]pyridin-5-yl)oxy)methyl)-2,4-difluorophenyl)-5-fluoro-2-methoxypyridine-3-sulfonamide (Compound 70)

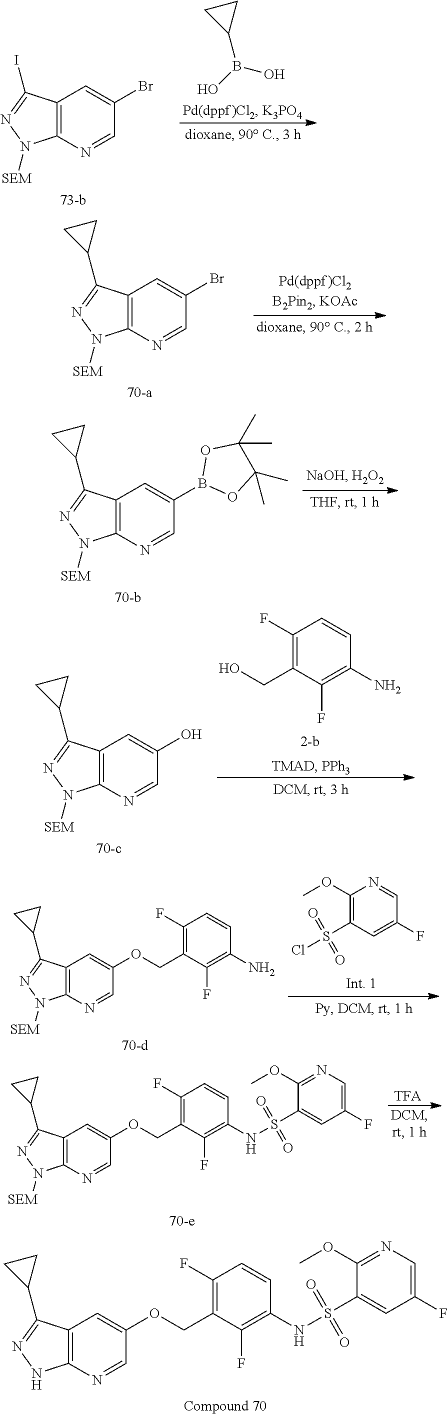

### Synthesis of 70-a: 5-bromo-3-cyclopropyl-1-((2-(trimethylsilyl)ethoxy)methyl)-1H-pyrazolo[3,4-b]pyridine

To a stirred solution of 5-bromo-3-iodo-1-[[2-(trimethylsilyl)ethoxy]methyl]pyrazolo[3,4-b]pyridine (500 mg, 1.1 mmol, 1 equiv) and cyclopropylboronic acid (104 mg, 1.2 mmol, 1.1 equiv) in dioxane (10 mL) were added K₃PO₄ (467 mg, 2.2 mmol, 2 equiv) and Pd(dppf)Cl₂ (81 mg, 0.11 mmol, 0.1 equiv) at room temperature, under nitrogen atmosphere. The reaction was stirred for 3 h at 90° C. then concentrated. The residue was purified by Flash-Prep-HPLC with the following conditions: Column, WelFlash TM C18-I, Spherical C18 20-40 μm, 120 g; mobile phase: 40-90% MeCN/0.1% aqueous formic acid; to afford 5-bromo-3-cyclopropyl-1-[[2-(trimethylsilyl)ethoxy]methyl]pyrazolo[3,4-b]pyridine (230 mg, 57% yield) as a brown oil.

LCMS (ES, m/z): [M+H]⁺: 368

### Synthesis of 70-b/c: 3-cyclopropyl-1-((2-(trimethylsilyl)ethoxy)methyl)-1H-pyrazolo[3,4-b]pyridin-5-ol

To a stirred solution of 5-bromo-3-cyclopropyl-1-[[2-(trimethylsilyl)ethoxy]methyl]pyrazolo[3,4-b]pyridine (220 mg, 0.6 mmol, 1 equiv) and 4,4,4′,4′,5,5,5′,5′-octamethyl-2,2′-bi(1,3,2-dioxaborolane) (156 mg, 0.66 mmol, 1.1 equiv) in dioxane (10 mL) were added KOAc (117 mg, 1.2 mmol, 2 equiv) and Pd(dppf)Cl₂ (44 mg, 0.06 mmol, 0.1 equiv) at room temperature, under nitrogen atmosphere. The reaction was stirred for 2 h at 90° C. then cooled to room temperature. THE (4 mL) was added, followed by 2% aqueous NaOH (4 mL) and 30% H₂O₂ (1.73 g, 15.3 mmol, 10 equiv) dropwise. The resulting mixture was stirred for 1 h at room temperature, then quenched with saturated aqueous Na₂S₂O₃ (10 mL) and extracted with EA (3×20 mL). The combined organics were washed with brine (10 mL), dried over anhydrous Na₂SO₄ and concentrated. The residue was purified by Flash-Prep-HPLC with the following conditions: Column, WelFlash TM C18-I, Spherical C18 20-40 μm, 120 g; mobile phase: 30-80% MeCN/0.1% aqueous formic acid; to afford 3-cyclopropyl-1-[[2-(trimethylsilyl)ethoxy]methyl]pyrazolo[3,4-b]pyridin-5-ol (140 mg, 76% yield) as a brown oil.

LCMS (ES, m/z): [M+H]⁺: 306

### Synthesis of 70-d: 3-(((3-cyclopropyl-1-((2-(trimethylsilyl)ethoxy)methyl)-1H-pyrazolo[3,4-b]pyridin-5-yl)oxy)methyl)-2,4-difluoroaniline

To a stirred solution of 3-cyclopropyl-1-[[2-(trimethylsilyl)ethoxy]methyl]pyrazolo[3,4-b]pyridin-5-ol (130 mg, 0.43 mmol, 1 equiv) and (3-amino-2,6-difluorophenyl)methanol (75 mg, 0.47 mmol, 1.1 equiv) in DCM (4 mL) were added TMAD (110 mg, 0.64 mmol, 1.5 equiv) and PPh₃ (167 mg, 0.64 mmol, 1.5 equiv). The reaction was stirred for 3 h, then concentrated. The residue was purified by Flash-Prep-HPLC with the following conditions: Column, WelFlash TM C18-I, Spherical C18 20-40 μm, 120 g; mobile phase: 50-95% MeCN/0.1% aqueous formic acid; to afford 3-[[(3-cyclopropyl-1-[[2-(trimethylsilyl)ethoxy]methyl]pyrazolo[3,4-b]pyridin-5-yl)oxy]methyl]-2,4-difluoroaniline (64 mg, 34% yield) as a brown oil.

LCMS (ES, m/z): [M+H]⁺: 447

### Synthesis of 70-e: N-(3-(((3-cyclopropyl-1-((2-(trimethylsilyl)ethoxy)methyl)-1H-pyrazolo[3,4-b]pyridin-5-yl)oxy)methyl)-2,4-difluorophenyl)-5-fluoro-2-methoxypyridine-3-sulfonamide

To a stirred solution of 3-[[(3-cyclopropyl-1-[[2-(trimethylsilyl)ethoxy]methyl]pyrazolo[3,4-b]pyridin-5-yl)oxy]methyl]-2,4-difluoroaniline (60 mg, 0.13 mmol, 1 equiv) in pyridine (2 mL) was added dropwise a solution of 5-fluoro-2-methoxypyridine-3-sulfonyl chloride (45 mg, 0.2 mmol, 1.5 equiv) in DCM (0.25 mL). The resulting mixture was stirred for 1 h, then concentrated. The residue was purified by Flash-Prep-HPLC with the following conditions: Column, WelFlash TM C18-I, Spherical C18 20-m, 120 g; mobile phase: 50-95% MeCN/0.1% aqueous formic acid; to afford N-(3-[[(3-cyclopropyl-1-[[2-(trimethylsilyl)ethoxy]methyl]pyrazolo[3,4-b]pyridin-5-yl)oxy]methyl]-2,4-difluorophenyl)-5-fluoro-2-methoxypyridine-3-sulfonamide (66 mg, 77% yield) as a brown oil.

LCMS (ES, m/z): [M+H]⁺: 636

### Synthesis of Compound 70: N-(3-(((3-cyclopropyl-1H-pyrazolo[3,4-b]pyridin-5-yl)oxy)methyl)-2,4-difluorophenyl)-5-fluoro-2-methoxypyridine-3-sulfonamide

A solution of N-(3-[[(3-cyclopropyl-1-[[2-(trimethylsilyl)ethoxy]methyl]pyrazolo[3,4-b]pyridin-5-yl)oxy]methyl]-2,4-difluorophenyl)-5-fluoro-2-methoxypyridine-3-sulfonamide (65 mg, 0.1 mmol, 1 equiv) in TFA (0.33 mL) and DCM (1 mL) was stirred for 1 h at room temperature, then concentrated. The residue was purified by prep-HPLC with the following conditions: Column, welch Vltimate XB—C18, 50×250 mm, 10 μm; Mobile Phase: 5-100% MeCN/0.1% aqueous formic acid; to afford N-[3-[([3-cyclopropyl-1H-pyrazolo[3,4-b]pyridin-5-yl]oxy)methyl]-2,4-difluorophenyl]-5-fluoro-2-methoxypyridine-3-sulfonamide (19 mg, 37% yield) as a light yellow solid.

LCMS (ES, m/z): [M+H]⁺: 506
¹H NMR (300 MHz, DMSO-d₆) δ 13.04 (s, 1H), 10.37 (s, 1H), 8.44 (d, J=3.0 Hz, 1H), 8.16 (d, J=2.8 Hz, 1H), 7.98 (dd, J=7.3, 3.0 Hz, 1H), 7.87 (d, J=2.8 Hz, 1H), 7.38 (td, J=8.8, 5.9 Hz, 1H), 7.22-7.10 (m, 1H), 5.15 (s, 2H), 3.90 (s, 3H), 2.25 (qd, J=7.9, 5.3 Hz, 1H), 0.98 (dp, J=7.8, 2.5 Hz, 4H).

### Example 82: Synthesis of N-[2,4-difluoro-3-[([1-methyl-2-oxo-3H-imidazo[4,5-b]pyridin-6-yl]oxy)methyl]phenyl]-5-fluoro-2-methoxypyridine-3-sulfonamide (Compound 71)

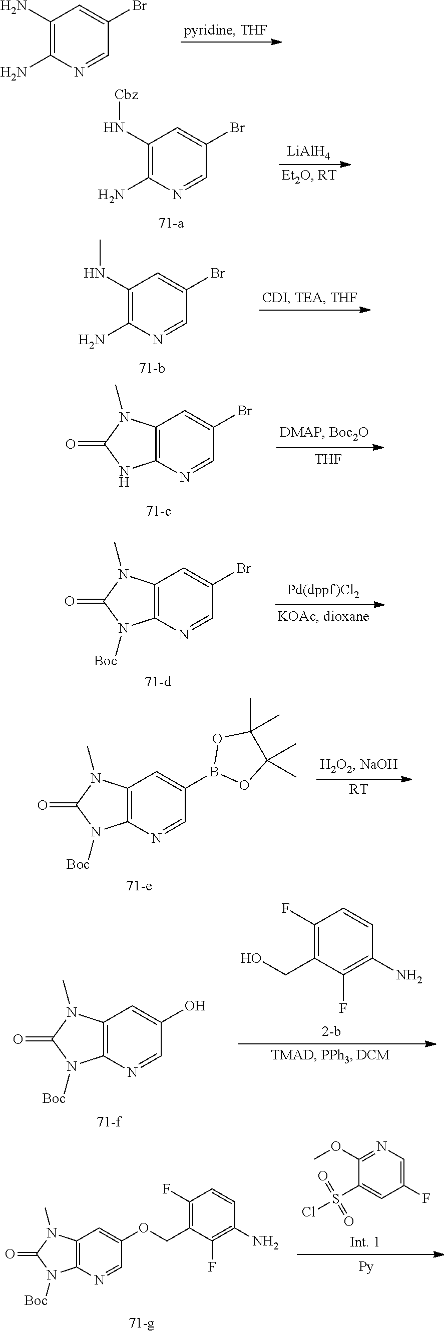

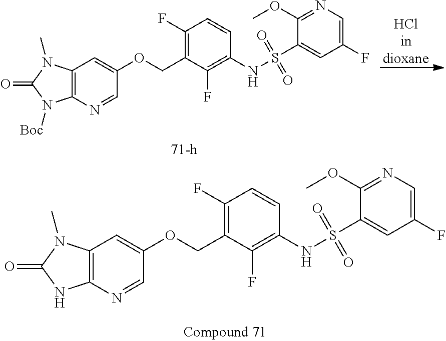 [SMILES: COc1ncc(F)cc1S(=O)(=O)Nc1ccc(F)c(COc2cnc3c(c2)n(C)c(=O)n3C(=O)OC(C)(C)C)c1F ; "HClindioxane" ; COc1ncc(F)cc1S(=O)(=O)Nc1ccc(F)c(COc2cnc3[nH]c(=O)n(C)c3c2)c1F]

### Synthesis of 71-a: benzyl N-(2-amino-5-bromopyridin-3-yl)carbamate

To a stirred solution of 5-bromopyridine-2,3-diamine (5 g, 27 mmol, 1 equiv) and pyridine (7.5 mL, 94 mmol, 3.5 equiv) in THE (125 mL) was added benzyl chloroformate (4.54 g, 27 mmol, 1 equiv) dropwise at 0° C. The reaction was stirred for 3 h then diluted with water (20 mL). The resulting mixture was extracted with EtOAc (3×50 mL), and the combined organics were washed with water, brine, dried over anhydrous Na₂SO₄, then concentrated. The residue was purified by silica gel column chromatography, eluting with EtOAc/PE (0-50%) to afford benzyl N-(2-amino-5-bromopyridin-3-yl)carbamate (3.7 g, 43% yield) as a yellow solid.

LCMS (ES, m/z): [M+H]⁺: 322.

### Synthesis of 71-b: 5-bromo-N³-methylpyridine-2,3-diamine

To a stirred mixture of LiAlH₄ (1.7 g, 45.9 mmol, 4 equiv) in Et₂O (40 mL) was added benzyl N-(2-amino-5-bromopyridin-3-yl)carbamate (3.7 g, 11.5 mmol, 1 equiv) in portions at 0° C. The reaction mixture was stirred overnight at room temperature, then cooled back to 0° C. MeOH (5 mL) was added followed by Na₂SO₄·10 H₂O (5 g) in portions. The resulting mixture was filtered, and the filter cake was washed with EtOAc (3×100 mL). The combined filtrate was concentrated and the residue was purified by silica gel column chromatography, eluting with EtOAc/PE (0-50%) to afford 5-bromo-N³-methylpyridine-2,3-diamine (1.8 g, 78% yield) as a yellow solid.

LCMS (ES, m/z): [M+H]⁺: 202.

### Synthesis of 71-c: 6-bromo-1-methyl-3H-imidazo[4,5-b]pyridin-2-one

A solution of 5-bromo-N³-methylpyridine-2,3-diamine (1.7 g, 8.4 mmol, 1 equiv), Et₃N (1.7 g, 17 mmol, 2 equiv) and CDI (2.05 g, 13 mmol, 1.5 equiv) in THE (20 mL) was stirred for 30 min, then heated for 3 h at 60° C. The mixture was allowed to cool to room temperature, then was diluted with water (50 mL). The resulting mixture was extracted with EtOAc (3×150 mL) and the combined organics were washed with water, brine, dried over anhydrous Na₂SO₄, then concentrated. The residue was purified by silica gel column chromatography, eluting with 0-70% EtOAc/PE to afford 6-bromo-1-methyl-3H-imidazo[4,5-b]pyridin-2-one (1.7 g, 89% yield) as a yellow solid.

LCMS (ES, m/z): [M+H]⁺: 228.

### Synthesis of 71-d: tert-butyl 6-bromo-1-methyl-2-oxoimidazo[4,5-b]pyridine-3-carboxylate

A solution of 6-bromo-1-methyl-3H-imidazo[4,5-b]pyridin-2-one (1.7 g, 7.5 mmol, 1 equiv), Boc₂O (2.44 g, 11 mmol, 1.5 equiv) and DMAP (182 mg, 1.5 mmol, 0.2 equiv) in MeCN (20 mL) was stirred overnight. The resulting mixture was diluted with water (20 mL) and extracted with EtOAc (3×50 mL). The combined organics were washed with water, brine, dried over anhydrous Na₂SO₄ and concentrated. The residue was purified by silica gel column chromatography, eluting with 0-30% EtOAc/PE to afford tert-butyl 6-bromo-1-methyl-2-oxoimidazo[4,5-b]pyridine-3-carboxylate (1.8 g, 74% yield) as a yellow solid.

LCMS (ES, m/z): [M+H]⁺: 328.

### Synthesis of 71-e: (tert-butyl 1-methyl-2-oxo-6-(4,4,5,5-tetramethyl-1,3,2-dioxaborolan-2-yl)imidazo[4,5-b]pyridine-3-carboxylate

A mixture of tert-butyl 6-bromo-1-methyl-2-oxoimidazo[4,5-b]pyridine-3-carboxylate (1 g, 3 mmol, 1 equiv), bis(pinacolato)diboron (1.2 g, 4.5 mmol, 1.5 equiv), KOAc (0.6 g, 6.1 mmol, 2 equiv) and Pd(dppf)Cl₂·CH₂Cl₂ (0.25 g, 0.3 mmol, 0.1 equiv) in dioxane (10 mL) was stirred for 2 h at 100° C. under nitrogen atmosphere. Desired product could be detected by LCMS. The resulting mixture was used in the next step directly without further purification.

LCMS (ES, m/z): [M+H]⁺: 376.

### Synthesis of 71-f: tert-butyl 6-hydroxy-1-methyl-2-oxoimidazo[4,5-b]pyridine-3-carboxylate

The solution of (tert-butyl-1-methyl-2-oxo-6-(4,4,5,5-tetramethyl-1,3,2-dioxaborolan-2-yl)imidazo[4,5-b]pyridine-3-carboxylate (2.9 mmol, 1 equiv) was diluted with THE (11 mL), and 2% aqueous NaOH (11 mL) was added, followed by 30% H₂O₂ (0.68 g, 5.9 mmol, 2 equiv) dropwise at room temperature. The reaction was stirred for 2 h then quenched with aqueous Na₂S₂O₄ (2 mL). The resulting mixture was extracted with EtOAc (3×30 mL). The combined organics were washed with water (2×30 mL) and brine (30 mL), dried over anhydrous Na₂SO₄ and concentrated. The residue was purified by silica gel column chromatography, eluting with 0-50% EtOAc/PE to afford tert-butyl 6-hydroxy-1-methyl-2-oxoimidazo[4,5-b]pyridine-3-carboxylate (450 mg, 57% yield) as a white solid.

LCMS (ES, m/z): [M+H]⁺: 266.

### Synthesis of 71-g: tert-butyl 6-[(3-amino-2,6-difluorophenyl)methoxy]-1-methyl-2-oxoimidazo[4,5-b]pyridine-3-carboxylate

A solution of tert-butyl 6-hydroxy-1-methyl-2-oxoimidazo[4,5-b]pyridine-3-carboxylate (450 mg, 1.7 mmol, 1 equiv), (3-amino-2,6-difluorophenyl)methanol (540 mg, 3.4 mmol, 2 equiv), TMAD (584 mg, 3.4 mmol, 2 equiv) and PPh₃ (890 mg, 3.4 mmol, 2 equiv) in DCM (4.5 mL) was stirred for 2 h then diluted with water (20 mL). The resulting mixture was extracted with DCM (3×50 mL). The combined organics were washed with water and brine, dried over anhydrous Na₂SO₄ and concentrated. The residue was purified by silica gel column chromatography, eluting with 0-50% EtOAc/PE to afford tert-butyl 6-[(3-amino-2,6-difluorophenyl)methoxy]-1-methyl-2-oxoimidazo[4,5-b]pyridine-3-carboxylate (306 mg, 44% yield) as a yellow solid.

LCMS (ES, m/z): [M+H]⁺: 407.

### Synthesis of 71-h: 6-[[2,6-difluoro-3-(5-fluoro-2-methoxypyridine-3-sulfonamido)phenyl]methoxy]-1-methyl-2-oxoimidazo[4,5-b]pyridine-3-carboxylate

To a stirred solution of tert-butyl 6-[(3-amino-2,6-difluorophenyl)methoxy]-1-methyl-2-oxoimidazo[4,5-b]pyridine-3-carboxylate (150 mg, 0.37 mmol, 1 equiv) in pyridine (1.5 mL) was added 5-fluoro-2-methoxypyridine-3-sulfonyl chloride (166 mg, 0.74 mmol, 2 equiv). The reaction was stirred for 2 h, then diluted with water (5 mL). The resulting mixture was extracted with EtOAc (3×20 mL). The combined organics were washed with water and brine, dried over anhydrous Na₂SO₄ and concentrated. The residue was purified by silica gel column chromatography, eluting with 0-50% EtOAc/PE to afford tert-butyl 6-[[2,6-difluoro-3-(5-fluoro-2-methoxypyridine-3-sulfonamido)phenyl]methoxy]-1-methyl-2-oxoimidazo[4,5-b]pyridine-3-carboxylate (130 mg, 59% yield) as a yellow solid.

LCMS (ES, m/z): [M+H]⁺: 596.

### Synthesis of Compound 71: N-[2,4-difluoro-3-[([1-methyl-2-oxo-3H-imidazo[4,5-b]pyridin-6-yl]oxy)methyl]phenyl]-5-fluoro-2-methoxypyridine-3-sulfonamide

To a stirred of tert-butyl 6-[[2,6-difluoro-3-(5-fluoro-2-methoxypyridine-3-sulfonamido)phenyl]methoxy]-1-methyl-2-oxoimidazo[4,5-b]pyridine-3-carboxylate (130 mg, 0.22 mmol, 1 equiv) was added 4 M HCl in 1,4-dioxane (1.3 mL) dropwise at 0-5° C. The resulting mixture was stirred for 2 h at room temperature, then concentrated. The residue was neutralized to pH 7 with saturated aqueous NaHCO₃ solution, and extracted with EtOAc (3×10 mL). The combined organics were washed with water and brine, dried over anhydrous Na₂SO₄ and concentrated. The crude product was purified by Prep-HPLC with the following conditions: Column, Sunfire Prep C18 OBD Column, 50*250 mm 5 μm 10 nm; mobile phase: 15-60% MeCN/0.1% aqueous formic acid; to afford N-[2,4-difluoro-3-[([1-methyl-2-oxo-3H-imidazo[4,5-b]pyridin-6-yl]oxy)methyl]phenyl]-5-fluoro-2-methoxypyridine-3-sulfonamide (41 mg, 38% yield) as a white solid.

LCMS (ES, m/z): [M+H]⁺: 496.
¹H NMR (300 MHz, DMSO-d₆) δ11.36 (s, 1H), 10.36 (s, 1H), 8.45 (d, J=2.9 Hz, 1H), 7.97 (dd, J=7.3, 3.0 Hz, 1H), 7.61 (d, J=2.5 Hz, 1H), 7.43-7.29 (m, 1H), 7.29 (d, J=2.5 Hz, 1H), 7.14 (td, J=9.0, 1.6 Hz, 1H), 5.08 (s, 2H), 3.91 (s, 3H), 3.27 (s, 3H).

### Example 83: Synthesis of N-[2,4-difluoro-3-([2-oxo-1H-spiro[cyclopropane-1,3-pyrrolo[2,3-b]pyridin]-5-yloxy]methyl)phenyl]-5-fluoro-2-methoxypyridine-3-sulfonamide (Compound 72)

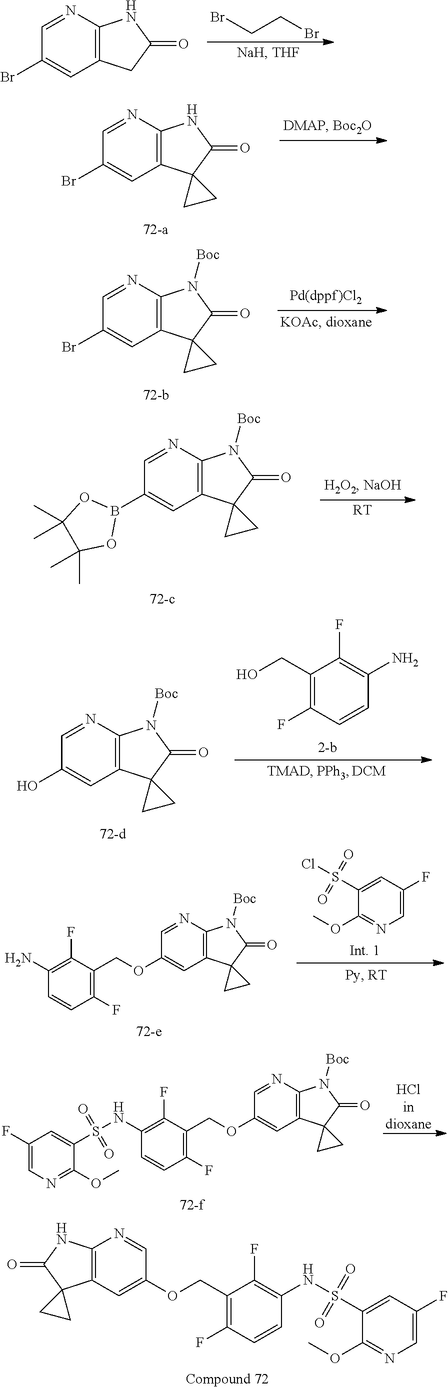

### Synthesis of 72-a: 5′-bromo-1′H-spiro[cyclopropane-1,3′-pyrrolo[2,3-b]pyridin]-2′-one

To a stirred solution of 5-bromo-1H,3H-pyrrolo[2,3-b]pyridin-2-one (5 g, 24 mmol, 1 equiv) and dibromoethane (13.2 g, 70 mmol, 3 equiv) in DMF (50 mL) was added NaH (5.63 g, 141 mmol, 6 equiv, 60% in oil) in portions at 0-5° C. The reaction was stirred for 2 h at room temperature, then cooled again to 0-5° C. and quenched with water (50 mL). The resulting mixture was extracted with EtOAc (3×150 mL). The combined organics were washed with water and brine, dried over anhydrous Na₂SO₄ and concentrated. The residue was purified by silica gel column chromatography, eluting with 0-40% EtOAc/PE to afford 5′-bromo-1′H-spiro[cyclopropane-1,3′-pyrrolo[2,3-b]pyridin]-2′-one (2.4 g, 43% yield) as a yellow solid.

LCMS (ES, m/z): [M+H]⁺: 239.

### Synthesis of 72-b: tert-butyl 5′-bromo-2′-oxospiro[cyclopropane-1,3′-pyrrolo[2,3-b]pyridine]-1′-carboxylate

A solution of 5′-bromo-1′H-spiro[cyclopropane-1,3′-pyrrolo[2,3-b]pyridin]-2′-one (2.3 g, 9.6 mmol, 1 equiv), di-tert-butyl dicarbonate (2.5 g, 12 mmol, 1.2 equiv) and DMAP (0.24 g, 1.9 mmol, 0.2 equiv) in MeCN (50 mL) was stirred overnight. The resulting mixture was diluted with water (20 mL) and extracted with EtOAc (3×50 mL). The combined organics were washed with water and brine, dried over anhydrous Na₂SO₄ and concentrated. The residue was purified by silica gel column chromatography, eluting with 0-30% EtOAc/PE to afford tert-butyl 5′-bromo-2′-oxospiro[cyclopropane-1,3′-pyrrolo[2,3-b]pyridine]-1′-carboxylate (2.4 g, 74% yield) as a yellow solid.

LCMS (ES, m/z): [M+H]⁺: 339.

### Synthesis of 72-c: (tert-butyl 2-oxo-5-(4,4,5,5-tetramethyl-1,3,2-dioxaborolan-2-yl)spiro[cyclopropane-1,3-pyrrolo[2,3-b]pyridine]-1-carboxylate

A mixture of tert-butyl 5′-bromo-2′-oxospiro[cyclopropane-1,3′-pyrrolo[2,3-b]pyridine]-1′-carboxylate (2.4 g, 7.1 mmol, 1 equiv), bis(pinacolato)diboron (2.7 g, 11 mmol, 1.5 equiv), KOAc (1.39 g, 14 mmol, 2 equiv) and Pd(dppf)Cl₂·CH₂Cl₂ (289 mg, 0.35 mmol, 0.05 equiv) in dioxane (25 mL) was stirred for 2 h at 100° C. Desired product could be detected by LCMS. The resulting mixture was used in the next step directly without further purification.

LCMS (ES, m/z): [M+H]⁺: 387.

### Synthesis of 72-d: tert-butyl 5-hydroxy-2-oxospiro[cyclopropane-1,3-pyrrolo[2,3-b]pyridine]-1-carboxylate

To the crude mixture (tert-butyl 2-oxo-5-(4,4,5,5-tetramethyl-1,3,2-dioxaborolan-2-yl)spiro[cyclopropane-1,3-pyrrolo[2,3-b]pyridine]-1-carboxylate (5.85 mmol, 1 equiv) were added THE (22 mL), 2% aqueous NaOH (22 mL) and 30% H₂O₂ (2.7 g, 23.4 mmol, 4 equiv) dropwise at room temperature. The reaction was stirred for 2 h then quenched with aqueous Na₂S₂O₄ (200 mL). The resulting mixture was extracted with EtOAc (3×150 mL). The combined organics were washed with water (2×150 mL) and brine (1×150 mL), dried over anhydrous Na₂SO₄ and concentrated. The residue was purified by silica gel column chromatography, eluting with 0-50% EtOAc/PE to afford tert-butyl 5-hydroxy-2-oxospiro[cyclopropane-1,3-pyrrolo[2,3-b]pyridine]-1-carboxylate (1 g, 62% yield) as a white solid.

LCMS (ES, m/z): [M+H]⁺: 277.

### Synthesis of 72-e: tert-butyl 5′-[(3-amino-2,6-difluorophenyl)methoxy]-2′-oxospiro[cyclopropane-1,3′-pyrrolo[2,3-b]pyridine]-1′-carboxylate

A solution of tert-butyl 5′-hydroxy-2′-oxospiro[cyclopropane-1,3′-pyrrolo[2,3-b]pyridine]-1′-carboxylate (1 g, 3.6 mmol, 1 equiv), (3-amino-2,6-difluorophenyl)methanol (0.63 g, 4 mmol, 1.1 equiv), TMAD (0.55 g, 5 mmol, 1.3 equiv) and PPh₃ (1.23 g, 5 mmol, 1.3 equiv) in DCM (10 mL) was stirred for 2 h then diluted with water (20 mL). The resulting mixture was extracted with DCM (3×50 mL). The combined organics were washed with water and brine, dried over anhydrous Na₂SO₄ and concentrated. The residue was purified by silica gel column chromatography, eluting with 0-60% EtOAc/PE to afford tert-butyl 5′-[(3-amino-2,6-difluorophenyl)methoxy]-2′-oxospiro[cyclopropane-1,3′-pyrrolo[2,3-b]pyridine]-1-carboxylate (700 mg, 46% yield) as a yellow solid

LCMS (ES, m/z): [M+H]⁺: 418.

### Synthesis of 72-f: tert-butyl 5′-[[2,6-difluoro-3-(5-fluoro-2-methoxypyridine-3-sulfonamido)phenyl]methoxy]-2′-oxospiro[cyclopropane-1,3′-pyrrolo[2,3-b]pyridine]-1′-carboxylate

To a stirred solution of tert-butyl 5′-[(3-amino-2,6-difluorophenyl)methoxy]-2′-oxospiro[cyclopropane-1,3′-pyrrolo[2,3-b]pyridine]-1′-carboxylate (150 mg, 0.36 mmol, 1 equiv) in pyridine (1.5 mL) was added 5-fluoro-2-methoxypyridine-3-sulfonyl chloride (162 mg, 0.72 mmol, 2 equiv). The reaction was stirred for 2 h then diluted with water (5 mL). The resulting mixture was extracted with EtOAc (3×20 mL). The combined organics were washed with water and brine, dried over anhydrous Na₂SO₄ and concentrated. The residue was purified by silica gel column chromatography, eluting with 0-100% EtOAc/PE to afford tert-butyl 5′-[[2,6-difluoro-3-(5-fluoro-2-methoxypyridine-3-sulfonamido)phenyl]methoxy]-2′-oxospiro[cyclopropane-1,3′-pyrrolo[2,3-b]pyridine]-1′-carboxylate (145 mg, 67% yield) as a yellow solid.

LCMS (ES, m/z): [M+H]⁺: 607.

### Synthesis of Compound 72: N-[2,4-difluoro-3-([2-oxo-1H-spiro[cyclopropane-1,3-pyrrolo[2,3-b]pyridin]-5-yloxy]methyl)phenyl]-5-fluoro-2-methoxypyridine-3-sulfonamide

Tert-butyl 5-[[2,6-difluoro-3-(5-fluoro-2-methoxypyridine-3-sulfonamido)phenyl]methoxy]-2-oxospiro[cyclopropane-1,3-pyrrolo[2,3-b]pyridine]-1-carboxylate (100 mg) was treated with 4 N HCl in 1,4-dioxane (1 mL) and stirred for 2 h at room temperature. The resulting mixture was concentrated under vacuum. The crude product was purified by Prep-HPLC with the following conditions: Column, Xselect CSH F-Phenyl OBD Column, 19*150 mm, 5 um; mobile phase: 15-42% MeCN/20 mmol/L NH₄HCO₃; to afford N-[2,4-difluoro-3-([2-oxo-1H-spiro[cyclopropane-1,3-pyrrolo[2,3-b]pyridin]-5-yloxy]methyl)phenyl]-5-fluoro-2-methoxypyridine-3-sulfonamide (12 mg, 14% yield) as a white solid.

LCMS (ES, m/z): [M+H]⁺: 507.
¹H NMR (300 MHz, DMSO-d₆) δ 11.00 (s, 1H), 8.30 (s, 1H), 7.90 (d, J=5.7 Hz, 1H), 7.77 (d, J=2.7 Hz, 1H), 7.31-7.19 (m, 1H), 6.94 (s, 1H), 5.01 (s, 2H), 3.83 (s, 3H), 1.63 (d, J=3.9 Hz, 2H), 1.51 (q, J=4.3, 3.7 Hz, 2H).

### Example 84: Synthesis of N-(3-(((3-cyano-1H-pyrazolo[3,4-b]pyridin-5-yl)oxy)methyl)-2,4-difluorophenyl)-5-fluoro-2-methoxypyridine-3-sulfonamide (Compound 73)

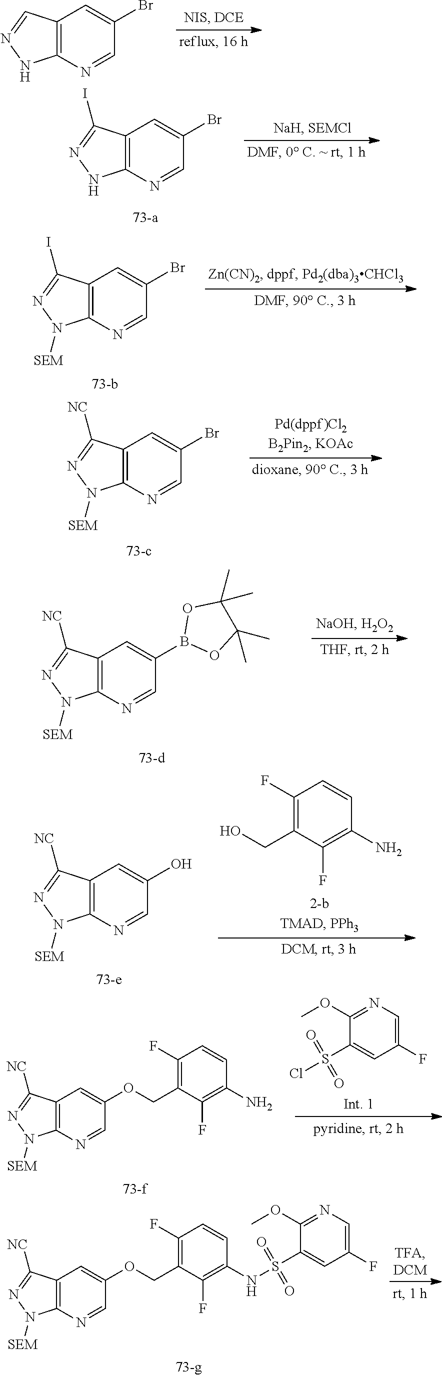

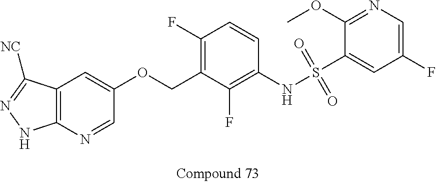 [SMILES: COc1ncc(F)cc1S(=O)(=O)Nc1ccc(F)c(COc2cnc3[nH]nc(C#N)c3c2)c1F]

### Synthesis of 73-a: 5-bromo-3-iodo-1H-pyrazolo[3,4-b]pyridine

A solution of 5-bromo-1H-pyrazolo[3,4-b]pyridine (10 g, 51 mmol, 1 equiv) and N-iodosuccinimide (11.9 g, 53 mmol, 1.05 equiv) in dry 1,2-dichloroethane (300 mL) was heated at reflux for 6 h. The reaction was cooled to room temperature and diluted with THF (300 mL). The resulting solution was washed with saturated aqueous Na₂S₂O₃ solution (2×100 mL), then brine (2×50 mL). The organic phase was dried over anhydrous Na₂SO₄ and concentrated. The residue was triturated with a 1:1 mixture of DCM/ether (50 mL), and then washed with ether (100 mL) to afford 5-bromo-3-iodo-1H-pyrazolo[3,4-b]pyridine as a yellow solid (12.4 g, crude).

LCMS (ES, m/z): [M+H]⁺: 324

### Synthesis of 73-b: 5-bromo-3-iodo-1-((2-(trimethylsilyl)ethoxy)methyl)-1H-pyrazolo[3,4-b]pyridine

To a stirred solution of 5-bromo-3-iodo-1H-pyrazolo[3,4-b]pyridine (5.2 g, 16 mmol, 1 equiv) in DMF (50 mL) was added 60% NaH in oil (1.93 g, 48 mmol, 3 equiv) in portions at 0° C. The reaction was stirred for 0.5 h at 0° C., then SEMCl (4 g, 24 mmol, 1.5 equiv) was added dropwise over 30 min at 0° C. The reaction was stirred for 1 h at room temperature, then quenched with water (300 mL). The resulting mixture was extracted with EA (3×100 mL). The combined organics were washed with brine (100 mL), dried over anhydrous Na₂SO₄ and concentrated. The residue was purified by silica gel column chromatography, eluting with PE:EA (20:1) to afford 5-bromo-3-iodo-1-[[2-(trimethylsilyl)ethoxy]methyl]pyrazolo[3,4-b]pyridine (3.2 g, 44% yield) as a white solid.

LCMS (ES, m/z): [M+H]⁺: 454

### Synthesis of 73-c: 5-bromo-1-((2-(trimethylsilyl)ethoxy)methyl)-1H-pyrazolo[3,4-b]pyridine-3-carbonitrile

To a stirred solution of 5-bromo-3-iodo-1-[[2-(trimethylsilyl)ethoxy]methyl]pyrazolo[3,4-b]pyridine (500 mg, 1.1 mmol, 1 equiv) and zinc cyanide (103 mg, 0.88 mmol, 0.8 equiv) in DMF (10 mL) were added dppf (122 mg, 0.22 mmol, 0.2 equiv) and Pd₂(dba)₃·CHCl₃ (114 mg, 0.11 mmol, 0.1 equiv) in portions at room temperature, under nitrogen atmosphere. The reaction was stirred for 3 h at 90° C., then cooled, filtered and the filtrate concentrated. The residue was purified by Flash-Prep-HPLC with the following conditions: Column, WelFlash TM C18-I, Spherical C18 20-40 μm, 120 g; mobile phase: 50-90% MeCN/0.1% aqueous formic acid; to afford 5-bromo-1-[[2-(trimethylsilyl)ethoxy]methyl]pyrazolo[3,4-b]pyridine-3-carbonitrile (260 mg, 67% yield) as a brown oil.

LCMS (ES, m/z): [M+H]⁺: 353

### Synthesis of 73-d/e: 5-hydroxy-1-((2-(trimethylsilyl)ethoxy)methyl)-1H-pyrazolo[3,4-b]pyridine-3-carbonitrile

To a stirred solution of 5-bromo-1-[[2-(trimethylsilyl)ethoxy]methyl]pyrazolo[3,4-b]pyridine-3-carbonitrile (255 mg, 0.72 mmol, 1 equiv) and bis(pinacolato)diboron (202 mg, 0.79 mmol, 1.1 equiv) in dioxane (8 mL) were added KOAc (142 mg, 1.44 mmol, 2 equiv) and Pd(dppf)Cl₂ (53 mg, 0.072 mmol, 0.1 equiv) in portions at room temperature, under nitrogen atmosphere. The reaction was stirred for 3 h at 90° C., then cooled. THE (4 mL) and 2% aqueous NaOH (4 mL) were added, followed by 30% H₂O₂ (246 mg, 7.24 mmol, 10 equiv) dropwise at room temperature. The reaction was stirred for 2 h, then quenched with saturated aqueous Na₂S₂O₃ (10 mL). The mixture was extracted with EA (3×20 mL). The combined organics were washed with brine (10 mL), dried over anhydrous Na₂SO₄ and concentrated. The residue was purified by Flash-Prep-HPLC with the following conditions: Column, WelFlash TM C18-I, Spherical C18 20-40 μm, 120 g; mobile phase: 30-80% MeCN/0.1% aqueous formic acid; to afford 5-hydroxy-1-[[2-(trimethylsilyl)ethoxy]methyl]pyrazolo[3,4-b]pyridine-3-carbonitrile (195 mg, 93% yield) as a brown oil.

LCMS (ES, m/z): [M+H]⁺: 291

### Synthesis of 73-f: 5-((3-amino-2,6-difluorobenzyl)oxy)-1-((2-(trimethylsilyl)ethoxy)methyl)-1H-pyrazolo[3,4-b]pyridine-3-carbonitrile

To a stirred solution of 5-hydroxy-1-[[2-(trimethylsilyl)ethoxy]methyl]pyrazolo[3,4-b]pyridine-3-carbonitrile (180 mg, 0.62 mmol, 1 equiv) and (3-amino-2,6-difluorophenyl)methanol (108 mg, 0.68 mmol, 1.1 equiv) in DCM (8 mL) were added TMAD (160 mg, 0.93 mmol, 1.5 equiv) and PPh₃ (244 mg, 0.93 mmol, 1.5 equiv). The reaction was stirred for 3 h then concentrated. The residue was purified by Flash-Prep-HPLC with the following conditions: Column, WelFlash TM C18-I, Spherical C18 20-40 μm, 120 g; mobile phase: 50-95% MeCN/0.1% aqueous formic acid; to afford 5-[(3-amino-2,6-difluorophenyl)methoxy]-1-[[2-(trimethylsilyl)ethoxy]methyl]pyrazolo[3,4-b]pyridine-3-carbonitrile (148 mg, 55% yield) as a brown oil.

LCMS (ES, m/z): [M+H]⁺: 432

### Synthesis of 73-g: N-(3-(((3-cyano-1-((2-(trimethylsilyl)ethoxy)methyl)-1H-pyrazolo[3,4-b]pyridin-5-yl)oxy)methyl)-2,4-difluorophenyl)-5-fluoro-2-methoxypyridine-3-sulfonamide

To a stirred solution of 5-[(3-amino-2,6-difluorophenyl)methoxy]-1-[[2-(trimethylsilyl)ethoxy]methyl]pyrazolo[3,4-b]pyridine-3-carbonitrile (140 mg, 0.32 mmol, 1 equiv) in pyridine (3 mL) was added 5-fluoro-2-methoxypyridine-3-sulfonyl chloride (110 mg, 0.49 mmol, 1.5 equiv). The reaction was stirred for 2 h then concentrated. The residue was purified by Flash-Prep-HPLC with the following conditions: Column, WelFlash TM C18-I, Spherical C18 20-40 μm, 120 g; mobile phase: 50-95% MeCN/0.1% aqueous formic acid; to afford N-(3-[[(3-cyano-1-[[2-(trimethylsilyl)ethoxy]methyl]pyrazolo[3,4-b]pyridin-5-yl)oxy]methyl]-2,4-difluorophenyl)-5-fluoro-2-methoxypyridine-3-sulfonamide (190 mg, 94% yield) as a brown oil.

LCMS (ES, m/z): [M+H]⁺: 621

### Synthesis of Compound 73: N-(3-(((3-cyano-1H-pyrazolo[3,4-b]pyridin-5-yl)oxy)methyl)-2,4-difluorophenyl)-5-fluoro-2-methoxypyridine-3-sulfonamide

A solution of N-(3-[[(3-cyano-1-[[2-(trimethylsilyl)ethoxy]methyl]pyrazolo[3,4-b]pyridin-5-yl)oxy]methyl]-2,4-difluorophenyl)-5-fluoro-2-methoxypyridine-3-sulfonamide (190 mg, 0.31 mmol, 1 equiv) in TFA (1 mL) and DCM (3 mL) was stirred for 1 h at room temperature. The resulting mixture was concentrated and the residue was purified by prep-HPLC with the following conditions: Column, welch Vltimate XB—C18, 50×250 mm, 10 μm; Mobile Phase: 5-100% MeCN/0.1% aqueous formic acid; to afford N-[3-[([3-cyano-1H-pyrazolo[3,4-b]pyridin-5-yl]oxy)methyl]-2,4-difluorophenyl]-5-fluoro-2-methoxypyridine-3-sulfonamide (72 mg, 48% yield) as a white solid.

LCMS (ES, m/z): [M+H]⁺: 491
¹H NMR (300 MHz, DMSO-d₆) δ 14.94 (s, 1H), 10.39 (s, 1H), 8.41 (dd, J=8.3, 2.9 Hz, 2H), 8.08 (d, J=2.7 Hz, 1H), 7.97 (dd, J=7.4, 3.1 Hz, 1H), 7.40 (td, J=8.9, 6.0 Hz, 1H), 7.18 (t, J=8.7 Hz, 1H), 5.22 (s, 2H), 3.90 (s, 3H).

### Example 85: Synthesis of N-[2-chloro-4-fluoro-3-[([3-methyl-1H-pyrazolo[3,4-b]pyridin-5-yl]oxy)methyl]phenyl]-5-fluoro-2-methylpyridine-3-sulfonamide (Compound 74)

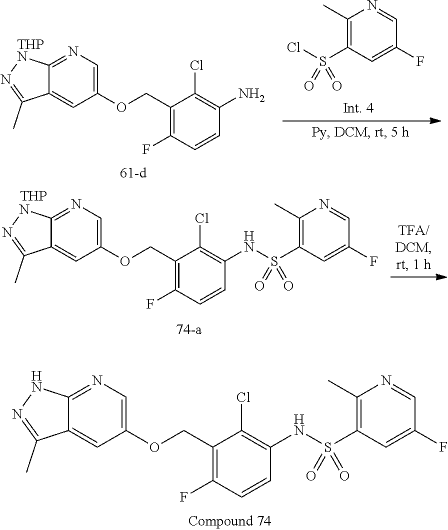

### Synthesis of 74-a: N-(2-chloro-4-fluoro-3-(((3-methyl-1-(tetrahydro-2H-pyran-2-yl)-1H-pyrazolo[3,4-b]pyridin-5-yl)oxy)methyl)phenyl)-5-fluoro-2-methylpyridine-3-sulfonamide

To a stirred solution of 2-chloro-4-fluoro-3-([[3-methyl-1-(oxan-2-yl)pyrazolo[3,4-b]pyridin-5-yl]oxy]methyl)aniline (300 mg, 0.7 mmol, 1 equiv) and pyridine (182 mg, 2.3 mmol, 3 equiv) in DCM (5 mL) was added 5-fluoro-2-methylpyridine-3-sulfonyl chloride (241 mg, 1.1 mmol, 1.5 equiv). The resulting mixture was stirred for 2 h then concentrated. The residue was purified by silica gel column chromatography, eluting with PE:EA (4:1) to afford N-[2-chloro-4-fluoro-3-([[3-methyl-1-(oxan-2-yl)pyrazolo[3,4-b]pyridin-5-yl]oxy]methyl)phenyl]-5-fluoro-2-methylpyridine-3-sulfonamide (120 mg, 28% yield) as a colorless oil.

LCMS (ES, m/z): [M+H]⁺: 564.

### Synthesis of Compound 74: N-[2-chloro-4-fluoro-3-[([3-methyl-1H-pyrazolo[3,4-b]pyridin-5-yl]oxy)methyl]phenyl]-5-fluoro-2-methylpyridine-3-sulfonamide

To a stirred solution of N-[2-chloro-4-fluoro-3-([[3-methyl-1-(oxan-2-yl)pyrazolo[3,4-b]pyridin-5-yl]oxy]methyl)phenyl]-5-fluoro-2-methylpyridine-3-sulfonamide (100 mg) in DCM (5 mL) was added TFA (1 mL). The resulting mixture was stirred for 2 h then concentrated. The crude product was purified by Prep-HPLC with following conditions: Column, welch Vltimate XB—C18, 50×250 mm, 10 μm; Mobile Phase: 20-60% MeCN/0.1% aqueous formic acid; to afford N-[2-chloro-4-fluoro-3-[([3-methyl-1H-pyrazolo[3,4-b]pyridin-5-yl]oxy)methyl]phenyl]-5-fluoro-2-methylpyridine-3-sulfonamide (50 mg, 59% yield) as a white solid.

LCMS (ES, m/z): [M+H]⁺: 480
¹H NMR (300 MHz, DMSO-d₆) δ 13.09 (s, 1H), 10.70 (s, 1H), 8.71 (d, J=2.8 Hz, 1H), 8.18 (d, J=2.7 Hz, 1H), 7.93-7.75 (m, 2H), 7.45-7.22 (m, 2H), 5.16 (s, 2H), 2.76 (s, 3H), 2.47 (s, 3H).

### Example 86: Synthesis of N-[3-([[3-(2,2-difluoroethyl)-1H-pyrazolo[3,4-b]pyridin-5-yl]oxy]methyl)-2,4-difluorophenyl]-5-fluoro-2-methoxypyridine-3-sulfonamide (Compound 75)

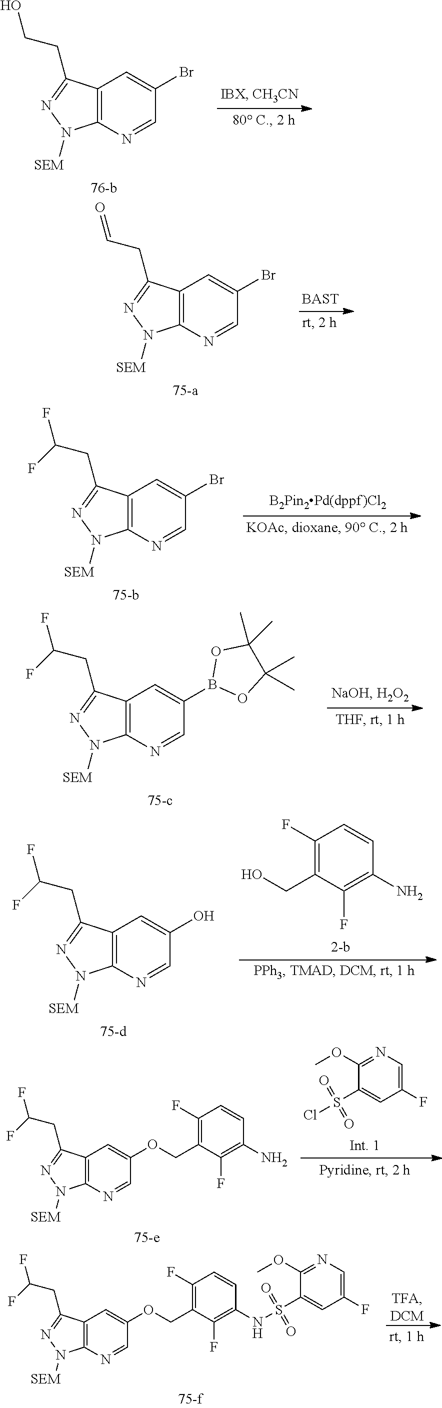

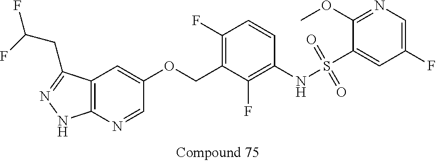 [SMILES: COc1ncc(F)cc1S(=O)(=O)Nc1ccc(F)c(COc2cnc3[nH]nc(CC(F)F)c3c2)c1F]

### Synthesis of 75-a: 2-(5-bromo-1-[[2-(trimethylsilyl)ethoxy]methyl]pyrazolo[3,4-b]pyridin-3-yl)acetaldehyde

Into a 40 mL vial was placed 2-(5-bromo-1-[[2-(trimethylsilyl)ethoxy]methyl]pyrazolo[3,4-b]pyridin-3-yl)ethanol (600 mg, 1.6 mmol, 1 equiv), CH₃CN (5 mL) and IBX (3.6 g, 13 mmol, 8 equiv). The resulting solution was stirred for 2 h at 80° C. then cooled and filtered. The filtrate was concentrated to give crude 2-(5-bromo-1-[[2-(trimethylsilyl)ethoxy]methyl]pyrazolo[3,4-b]pyridin-3-yl)acetaldehyde (400 mg) as off-white oil which was used in next step directly without further purification.

### Synthesis of 75-b: 5-bromo-3-(2,2-difluoroethyl)-1-[[2-(trimethylsilyl)ethoxy]methyl]pyrazolo[3,4-b]pyridine

Into a 40 mL vial was placed 2-(5-bromo-1-[[2-(trimethylsilyl)ethoxy]methyl]pyrazolo[3,4-b]pyridin-3-yl)acetaldehyde (400 mg, 1 mmol, 1 equiv), DCM (5 mL) and BAST (860 mg, 3.6 mmol, 3.6 equiv). The reaction was stirred for 2 h then diluted with NaHCO₃ solution (5 mL). The resulting mixture was extracted with ethyl acetate (3×5 mL) and the combined organics were dried over anhydrous sodium sulfate, then concentrated. The residue was purified by prep-HPLC with the following conditions: Column, WelFlash TM C18-I, Spherical C18 20-40 μm, 120 g; mobile phase: 60-95% MeCN/0.1% aqueous formic acid; to afford 5-bromo-3-(2,2-difluoroethyl)-1-[[2-(trimethylsilyl)ethoxy]methyl]pyrazolo[3,4-b]pyridine (300 mg, 71% yield) as a yellow oil.

LCMS (ES, m/z): [M+H]⁺: 392

### Synthesis of 75-c: 3-(2,2-difluoroethyl)-1-methyl-5-(4,4,5,5-tetramethyl-1,3,2-dioxaborolan-2-yl)pyrazolo[3,4-b]pyridine

To a stirred solution of 5-bromo-3-(2,2-difluoroethyl)-1-[[2-(trimethylsilyl)ethoxy]methyl]pyrazolo[3,4-b]pyridine (300 mg, 0.8 mmol, 1 equiv) and 4,4,5,5-tetramethyl-2-(tetramethyl-1,3,2-dioxaborolan-2-yl-1,3,2-dioxaborolane (213 mg, 0.8 mmol, 1 equiv) in dioxane (4 mL) was added Pd(dppf)Cl₂ (62 mg, 0.08 mmol, 0.1 equiv) and KOAc (150 mg, 1.6 mmol, 2 equiv) in portions at room temperature under nitrogen atmosphere. The resulting solution was stirred for 2 h at 90° C. then concentrated under vacuum to give crude 3-(2,2-difluoroethyl)-1-methyl-5-(4,4,5,5-tetramethyl-1,3,2-dioxaborolan-2-yl)pyrazolo[3,4-b]pyridine (240 mg) as brown oil which was used in next step directly without further purification.

LCMS (ES, m/z): [M+H]⁺: 440

### Synthesis of 75-d: 3-(2,2-difluoroethyl)-1-[[2-(trimethylsilyl)ethoxy]methyl]pyrazolo[3,4-b]pyridin-5-ol

To crude 3-(2,2-difluoroethyl)-1-methyl-5-(4,4,5,5-tetramethyl-1,3,2-dioxaborolan-2-yl)pyrazolo[3,4-b]pyridine (240 mg) was added THE (4 mL) and 2% aqueous NaOH (4 mL), followed by 30% H₂O₂ (139 mg, 4 mmol, 10 equiv) dropwise at room temperature. The resulting mixture was stirred for 1 h then quenched with saturated aqueous Na₂S₂O₃ (10 mL) and extracted with EA (3×10 mL). The combined organics were washed with brine (5 mL), dried over anhydrous Na₂SO₄ and concentrated. The residue was purified by Flash-Prep-HPLC with the following conditions: Column, WelFlash TM C18-I, Spherical C18 20-40 μm, 120 g; mobile phase: 30-80% MeCN/0.1% aqueous formic acid; to afford 3-(2,2-difluoroethyl)-1-[[2-(trimethylsilyl)ethoxy]methyl]pyrazolo[3,4-b]pyridin-5-ol (200 mg, 95% yield) as a brown oil.

LCMS (ES, m/z): [M+H]⁺: 330

### Synthesis of 75-e: 3-([[3-(2,2-difluoroethyl)-1-[[2-(trimethylsilyl)ethoxy]methyl]pyrazolo[3,4-b]pyridin-5-yl]oxy]methyl)-2,4-difluoroaniline

To a stirred solution of 3-(2,2-difluoroethyl)-1-[[2-(trimethylsilyl)ethoxy]methyl]pyrazolo[3,4-b]pyridin-5-ol (220 mg, 0.7 mmol, 1 equiv) in DCM (6 mL) was added (3-amino-2,6-difluorophenyl)methanol (117 mg, 0.7 mmol, 1 equiv), TMAD (138 mg, 0.8 mmol, 1.2 equiv) and PPh₃ (210 mg, 0.8 mmol, 1.2 equiv). The reaction was stirred for 1 h then diluted with H₂O (5 mL). The resulting mixture was extracted with ethyl acetate (3×5 mL), and the combined organics were dried over anhydrous sodium sulfate, then concentrated. The residue was purified by Flash-Prep-HPLC with the following conditions: Column, WelFlash TM C18-I, Spherical C18 20-40 μm, 120 g; mobile phase: 30-80% MeCN/0.1% aqueous formic acid; to afford 3-([[3-(2,2-difluoroethyl)-1-[[2-(trimethylsilyl)ethoxy]methyl]pyrazolo[3,4-b]pyridin-5-yl]oxy]methyl)-2,4-difluoroaniline (200 mg, 64% yield) as a brown solid.

LCMS (ES, m/z): [M+H]⁺: 471

### Synthesis of 75-f: N-[3-([[3-(2,2-difluoroethyl)-1-[[2-(trimethylsilyl)ethoxy]methyl]pyrazolo[3,4-b]pyridin-5-yl]oxy]methyl)-2,4-difluorophenyl]-5-fluoro-2-methoxypyridine-3-sulfonamide

Into an 8 mL vial was placed 3-([[3-(2,2-difluoroethyl)-1-[[2-(trimethylsilyl)ethoxy]methyl]pyrazolo[3,4-b]pyridin-5-yl]oxy]methyl)-2,4-difluoroaniline (190 mg, 0.4 mmol, 1 equiv), pyridine (2 mL) and 5-fluoro-2-methoxypyridine-3-sulfonyl chloride (110 mg, 0.5 mmol, 1.2 equiv). The resulting solution was stirred for 1 h then concentrated. The residue was purified by Flash-Prep-HPLC with the following conditions: Column, WelFlash TM C18-I, Spherical C18 20-40 μm, 120 g; mobile phase: 30-80% MeCN/0.1% aqueous formic acid; to afford N-[3-([[3-(2,2-difluoroethyl)-1-[[2-(trimethylsilyl)ethoxy]methyl]pyrazolo[3,4-b]pyridin-5-yl]oxy]methyl)-2,4-difluorophenyl]-5-fluoro-2-methoxypyridine-3-sulfonamide (230 mg, 86% yield) as a brown oil.

LCMS (ES, m/z): [M+H]⁺: 660

### Synthesis of Compound 75: N-[3-([[3-(2,2-difluoroethyl)-1H-pyrazolo[3,4-b]pyridin-5-yl]oxy]methyl)-2,4-difluorophenyl]-5-fluoro-2-methoxypyridine-3-sulfonamide

Into a 40 mL vial was placed N-[3-([[3-(2,2-difluoroethyl)-1-[[2-(trimethylsilyl)ethoxy]methyl]pyrazolo[3,4-b]pyridin-5-yl]oxy]methyl)-2,4-difluorophenyl]-5-fluoro-2-methoxypyridine-3-sulfonamide (230 mg, 0.3 mmol, 1 equiv), DCM (3 mL) and TFA (1 mL). The resulting solution was stirred for 1 h then concentrated. The residue was purified by prep-HPLC with the following conditions: Column, welch Vltimate XB—C18, 50×250 mm, 10 μm; Mobile Phase: 25-65% MeCN/0.1% aqueous formic acid; to afford N-[3-([[3-(2,2-difluoroethyl)-1H-pyrazolo[3,4-b]pyridin-5-yl]oxy]methyl)-2,4-difluorophenyl]-5-fluoro-2-methoxypyridine-3-sulfonamide (78 mg, 42% yield) as a white solid.

LCMS (ES, m/z): [M+H]⁺: 530
¹H NMR (300 MHz, DMSO-d₆) δ 13.48 (s, 1H), 10.37 (s, 1H), 8.45 (d, J=3.0 Hz, 1H), 8.23 (d, J=2.8 Hz, 1H), 8.02-7.90 (m, 2H), 7.38 (td, J=9.0, 6.0 Hz, 1H), 7.16 (t, J=9.0 Hz, 1H), 6.44 (t, J=4.5 Hz, 1H), 5.13 (s, 2H), 3.91 (s, 3H), 3.53 (td, J=17.7, 4.5 Hz, 2H).

### Example 87: Synthesis of N-[2,4-difluoro-3-([[3-(2-fluoroethyl)-1H-pyrazolo[3,4-b]pyridin-5-yl]oxy]methyl)phenyl]-5-fluoro-2-methoxypyridine-3-sulfonamide (Compound 76)

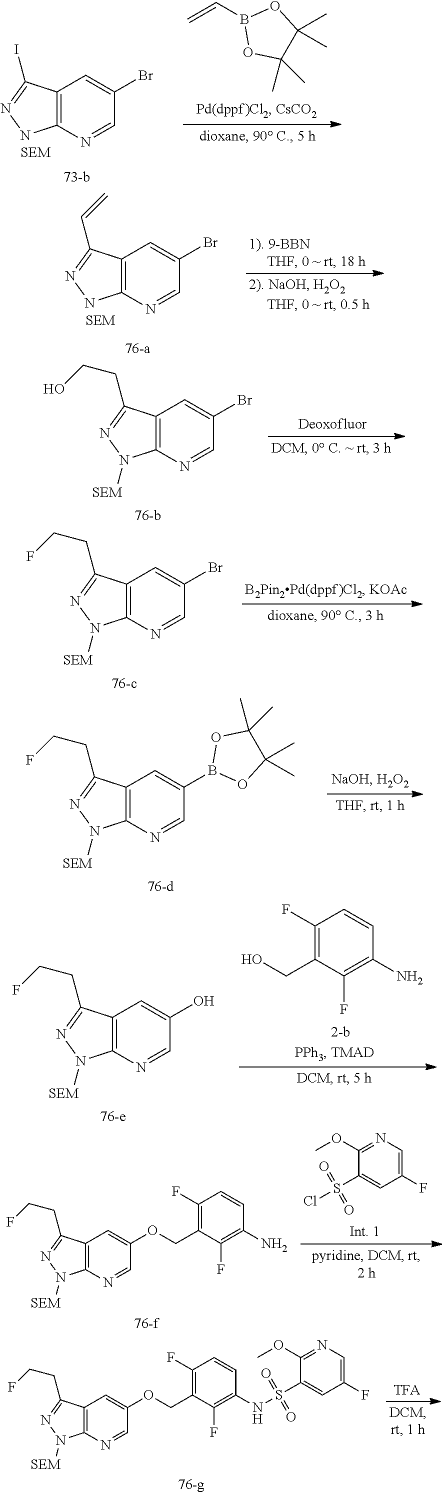

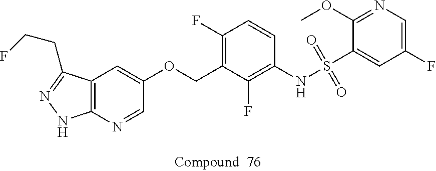 [SMILES: COc1ncc(F)cc1S(=O)(=O)Nc1ccc(F)c(COc2cnc3[nH]nc(CCF)c3c2)c1F]

### Synthesis of 76-a: 5-bromo-3-ethenyl-1-[[2-(trimethylsilyl)ethoxy]methyl]pyrazolo[3,4-b]pyridine

To a stirred solution of 5-bromo-3-iodo-1-[[2-(trimethylsilyl)ethoxy]methyl]pyrazolo[3,4-b]pyridine (1 g, 2.2 mmol, 1 equiv) and 2-ethenyl-4,4,5,5-tetramethyl-1,3,2-dioxaborolane (0.41 g, 2.64 mmol, 1.2 equiv) in dioxane (10 mL) were added Cs₂CO₃ (2.15 g, 6.6 mmol, 3 equiv) and Pd(dppf)Cl₂ (0.16 g, 0.22 mmol, 0.1 equiv) under nitrogen atmosphere. The reaction was stirred for 5 h at 90° C., before being cooled and quenched with H₂O (30 mL), then extracted with EtOAc (3×10 mL). The combined organics were washed with brine (30 mL), dried over anhydrous Na₂SO₄ and concentrated. The residue was purified by silica gel column chromatography, eluting with PE/EtOAc (5:1) to afford 5-bromo-3-ethenyl-1-[[2-(trimethylsilyl)ethoxy]methyl]pyrazolo[3,4-b]pyridine (400 mg, 51% yield) as a white oil.

### Synthesis of 76-b: 2-(5-bromo-1-[[2-(trimethylsilyl)ethoxy]methyl]pyrazolo[3,4-b]pyridin-3-yl)ethanol

To a stirred solution of 5-bromo-3-ethenyl-1-[[2-(trimethylsilyl)ethoxy]methyl]pyrazolo[3,4-b]pyridine (1.5 g, 4.23 mmol, 1 equiv) in THF (15 mL) was added 9-borabicyclo[3.3.1]nonane (2.07 g, 16.93 mmol, 4 equiv) dropwise at 0° C. The reaction was stirred for 18 h at room temperature, then cooled back to 0° C. and quenched with 2% aqueous NaOH (15 mL) dropwise over 1 min. 30% H₂O₂ (1.44 g, 42.33 mmol, 10 equiv) was added dropwise over 2 min and the reaction stirred to room temperature over 30 mins. Saturated aqueous Na₂S₂O₃ (10 mL) was added, and the mixture extracted with EA (3×10 mL). The combined organics were washed with brine (5 mL), dried over anhydrous Na₂SO₄ and concentrated. The residue was purified by silica gel column chromatography, eluting with PE:EA (3:1) to afford 2-(5-bromo-1-[[2-(trimethylsilyl)ethoxy]methyl]pyrazolo[3,4-b]pyridin-3-yl)ethanol (1.12 g, 71% yield) as a brown oil.

LCMS (ES, m/z): [M+H]⁺: 372

### Synthesis of 76-c: 5-bromo-3-(2-fluoroethyl)-1-[[2-(trimethylsilyl)ethoxy]methyl]pyrazolo[3,4-b]pyridine

To a stirred solution of 2-(5-bromo-1-[[2-(trimethylsilyl)ethoxy]methyl]pyrazolo[3,4-b]pyridin-3-yl)ethanol (500 mg, 1.3 mmol, 1 equiv) in DCM (10 mL) was added BAST (2.97 g, 13 mmol, 10 equiv) in portions at 0° C. The reaction was stirred for 3 h then concentrated. The residue was purified by Flash-Prep-HPLC with the following conditions: Column, WelFlash TM C18-I, Spherical C18 20-40 μm, 120 g; mobile phase: 50-95% MeCN/0.1% aqueous formic acid; to afford 5-bromo-3-(2-fluoroethyl)-1-[[2-(trimethylsilyl)ethoxy]methyl]pyrazolo[3,4-b]pyridine (380 mg, 76% yield) as a yellow oil.

LCMS (ES, m/z): [M+H]⁺: 374

### Synthesis of 76-d: 3-(2-fluoroethyl)-5-(4,4,5,5-tetramethyl-1,3,2-dioxaborolan-2-yl)-1-[[2-(trimethylsilyl)ethoxy]methyl]pyrazolo[3,4-b]pyridine

To a stirred solution of 5-bromo-3-(2-fluoroethyl)-1-[[2-(trimethylsilyl)ethoxy]methyl]pyrazolo[3,4-b]pyridine (39 mg, 0.1 mmol, 1 equiv) and bis(pinacolato)diboron (40 mg, 0.16 mmol, 1.5 equiv) in dioxane (2 mL) were added KOAc (21 mg, 0.21 mmol, 2 equiv) and Pd(dppf)Cl₂ (73 mg, 0.1 mmol, 0.1 equiv) at room temperature, under nitrogen atmosphere. The reaction was stirred for 3 h at 90° C. then cooled and quenched with H₂O (20 mL). This was extracted with EtOAc (3×10 mL), and the combined organics were washed with brine (20 mL), dried over anhydrous Na₂SO₄ then concentrated to afford 3-(2-fluoroethyl)-5-(4,4,5,5-tetramethyl-1,3,2-dioxaborolan-2-yl)-1-[[2-(trimethylsilyl)ethoxy]methyl]pyrazolo[3,4-b]pyridine (400 mg, 94% yield) as a brown oil, which was used in the next step directly without further purification.

LCMS (ES, m/z): [M+H]⁺: 422

### Synthesis of 76-e: 3-(2-fluoroethyl)-1-[[2-(trimethylsilyl)ethoxy]methyl]pyrazolo[3,4-b]pyridin-5-ol

To 3-(2-fluoroethyl)-5-(4,4,5,5-tetramethyl-1,3,2-dioxaborolan-2-yl)-1-[[2-(trimethylsilyl)ethoxy]methyl]pyrazolo[3,4-b]pyridine (400 mg, 0.95 mmol, 1 equiv) in tetrahydrofuran (4 mL) was added 2% aqueous NaOH solution (4 mL) and 30% H₂O₂ (0.32 g, 9.49 mmol, 10 equiv) dropwise at room temperature. The reaction was stirred for 1 h at room temperature, then quenched with H₂O (20 mL) and extracted with EtOAc (3×10 mL). The combined organics were washed with brine (20 mL), dried over anhydrous Na₂SO₄ and concentrated. The residue was purified by Flash-Prep-HPLC with the following conditions: Column, WelFlash TM C18-I, Spherical C18 20-40 μm, 120 g; mobile phase: 30-80% MeCN/0.1% aqueous formic acid; to afford 3-(2-fluoroethyl)-1-[[2-(trimethylsilyl)ethoxy]methyl]pyrazolo[3,4-b]pyridin-5-ol (290 mg, 98% yield) as a brown oil.

LCMS (ES, m/z): [M+H]⁺: 312

### Synthesis of 76-f: 2,4-difluoro-3-([[3-(2-fluoroethyl)-1-[[2-(trimethylsilyl)ethoxy]methyl]pyrazolo[3,4-b]pyridin-5-yl]oxy]methyl)aniline

A solution of 3-(2-fluoroethyl)-1-[[2-(trimethylsilyl)ethoxy]methyl]pyrazolo[3,4-b]pyridin-5-ol (290 mg, 0.93 mmol, 1 equiv), (3-amino-2,6-difluorophenyl)methanol (178 mg, 1.1 mmol, 1.2 equiv), PPh₃ (366 mg, 1.4 mmol, 1.5 equiv) and TMAD (241 mg, 1.4 mmol, 1.5 equiv) in DCM (10 mL) was stirred for 5 h at room temperature. The resulting mixture was quenched with H₂O (20 mL) and extracted with EtOAc (3×10 mL). The combined organics were washed with brine (20 mL), dried over anhydrous Na₂SO₄ and concentrated. The residue was purified by Flash-Prep-HPLC with the following conditions: Column, WelFlash TM C18-I, Spherical C18 20-40 μm, 120 g; mobile phase: 40-90% MeCN/0.1% aqueous formic acid; to afford 2,4-difluoro-3-([[3-(2-fluoroethyl)-1-[[2-(trimethylsilyl)ethoxy]methyl]pyrazolo[3,4-b]pyridin-5-yl]oxy]methyl)aniline (343 mg, 81% yield) as a brown oil.

LCMS (ES, m/z): [M+H]⁺: 453

### Synthesis of 76-g: N-[2,4-difluoro-3-([[3-(2-fluoroethyl)-1-[[2-(trimethylsilyl)ethoxy]methyl]pyrazolo[3,4-b]pyridin-5-yl]oxy]methyl)phenyl]-5-fluoro-2-methoxypyridine-3-sulfonamide

To a stirred solution of 2,4-difluoro-3-([[3-(2-fluoroethyl)-1-[[2-(trimethylsilyl)ethoxy]methyl]pyrazolo[3,4-b]pyridin-5-yl]oxy]methyl)aniline (348 mg, 0.77 mmol, 1 equiv) in pyridine (5 mL) was added 5-fluoro-2-methoxypyridine-3-sulfonyl chloride (260 mg, 1.2 mmol, 1.5 equiv) in DCM (0.5 mL) dropwise. The reaction was stirred for 2 h then concentrated to afford crude N-[2,4-difluoro-3-([[3-(2-fluoroethyl)-1-[[2-(trimethylsilyl)ethoxy]methyl]pyrazolo[3,4-b]pyridin-5-yl]oxy]methyl)phenyl]-5-fluoro-2-methoxypyridine-3-sulfonamide (450 mg, 91% yield) as a brown oil which was used in next step directly without further purification.

LCMS (ES, m/z): [M+H]⁺: 642

### Synthesis of Compound 76: N-[2,4-difluoro-3-([[3-(2-fluoroethyl)-1H-pyrazolo[3,4-b]pyridin-5-yl]oxy]methyl)phenyl]-5-fluoro-2-methoxypyridine-3-sulfonamide

A solution of N-[2,4-difluoro-3-([[3-(2-fluoroethyl)-1-[[2-(trimethylsilyl)ethoxy]methyl]pyrazolo[3,4-b]pyridin-5-yl]oxy]methyl)phenyl]-5-fluoro-2-methoxypyridine-3-sulfonamide (490 mg, 0.76 mmol, 1 equiv) in TFA (0.5 mL) and DCM (5 mL) was stirred for 1 h then concentrated. The residue was purified by prep-HPLC with the following conditions: Column, welch Vltimate XB—C18, 50×250 mm, 10 μm; Mobile Phase: 30-70% MeCN/0.1% aqueous formic acid; to afford N-[2,4-difluoro-3-([[3-(2-fluoroethyl)-1H-pyrazolo[3,4-b]pyridin-5-yl]oxy]methyl)phenyl]-5-fluoro-2-methoxypyridine-3-sulfonamide (82 mg, 21% yield) as a white solid.

LCMS (ES, m/z): [M+H]⁺: 512
¹H NMR (300 MHz, DMSO-d₆) δ 13.30 (s, 1H), 10.37 (s, 1H), 8.45 (d, J=2.9 Hz, 1H), 8.20 (d, J=2.8 Hz, 1H), 8.02-7.89 (m, 2H), 7.38 (td, J=8.9, 6.1 Hz, 1H), 7.16 (t, J=8.3 Hz, 1H), 5.13 (s, 2H), 4.90 (t, J=6.5 Hz, 1H), 4.74 (t, J=6.5 Hz, 1H), 3.91 (s, 3H), 3.39-3.21 (m, 2H).

### Example 88: Synthesis of N-(4-chloro-2-fluoro-3-(((3-methyl-1H-pyrazolo[3,4-b]pyridin-5-yl)oxy)methyl)phenyl)-5-fluoro-2-methylpyridine-3-sulfonamide (Compound 77)

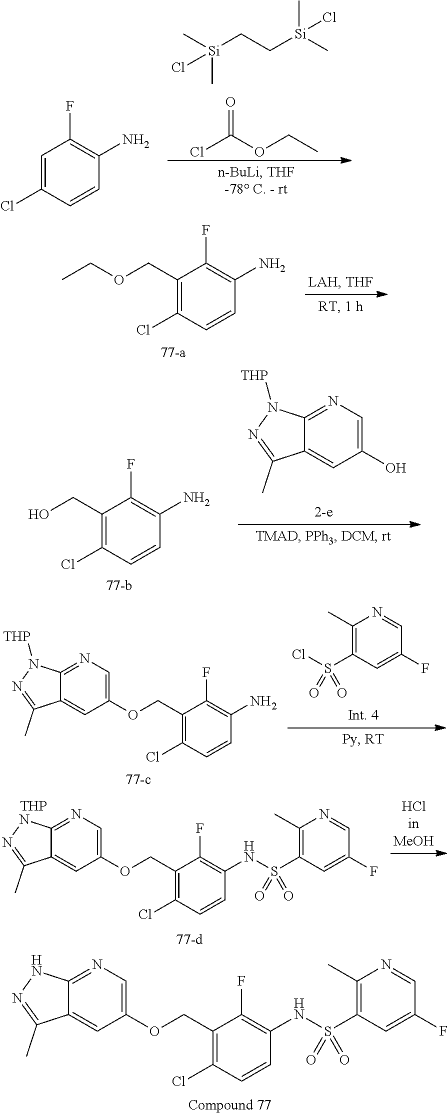

### Synthesis of 77-a: ethyl 3-amino-6-chloro-2-fluorobenzoate

4-Chloro-2-fluoroaniline (1 g, 6.9 mmol, 1 equiv) was dissolved in THE (2 mL) and cooled to −78° C. 2.5 M n-Butyllithium in hexanes (3 mL, 7.6 mmol) was slowly added dropwise, keeping the temperature below −70° C. The mixture was stirred at −70° C. for 30 minutes, then chloro[2-(chlorodimethylsilyl)ethyl]dimethylsilane (1.55 g, 7.2 mmol, 1.05 equiv) in THE (1 mL) was slowly added dropwise to the reaction mixture while keeping the temperature below −70° C. Once addition was complete, the mixture was stirred at −78° C. for 1 hour, then further 2.5 M n-butyllithium (3 mL, 7.6 mmol) was slowly added dropwise. The mixture was stirred for 30 minutes at −78° C., then allowed to warm to 15° C. over 1 hour. The reaction mixture was cooled to −78° C. again, and 2.5 M n-butyllithium (3 mL, 7.6 mmol) was slowly added dropwise. The mixture was stirred below −70° C. for 90 minutes, then ethyl chloroformate (970 mg, 8.9 mmol, 1.3 equiv) was added dropwise, again maintaining the temperature below −70° C. The cooling bath was removed and the reaction mixture was allowed to reach room temperature over 16 hrs. The reaction was cooled in an ice/water bath and quenched by addition of 3 M hydrochloric acid (50 mL). The mixture was stirred at room temperature for 2 hours, then made basic by addition of potassium carbonate solution. The mixture was extracted with ethyl acetate (3×100 mL), and the combined organics were washed with brine (50 mL), dried over magnesium sulfate and concentrated. The residue was purified with silica gel column chromatography eluting with 0-100% ethyl acetate in hexane to provide ethyl 3-amino-6-chloro-2-fluorobenzoate (1.3 g, 87% yield) as a light yellow oil.

LCMS (ES, m/z): [M+H]⁺: 218.

### Synthesis of 77-b: (3-amino-6-chloro-2-fluorophenyl)methanol

To a solution of ethyl 3-amino-6-chloro-2-fluorobenzoate (1.1 g, 5.1 mmol, 1 equiv) in THE (20 mL) was added LiAlH₄ (0.38 g, 0.12 mmol, 2 equiv) and the solution stirred for 1 h at room temperature. The reaction was cooled in an ice bath then quenched by the addition of MeOH (5 mL). Na₂SO₄·10H₂O (1 g) was added and the resulting mixture was stirred for 4 h. The mixture was filtered, and the filter cake washed with EA (3×20 mL). The combined filtrate was concentrated and the residue purified by silica gel column chromatography, eluting with PE/EA (1/1) to afford (3-amino-6-chloro-2-fluorophenyl)methanol (918 mg, 94% yield) as a light yellow solid.

LCMS (ES, m/z): [M+H]⁺: 176.

### Synthesis of 77-c: 4-chloro-2-fluoro-3-(((3-methyl-1-(tetrahydro-2H-pyran-2-yl)-1H-pyrazolo[3,4-b]pyridin-S-yl)oxy)methyl)aniline

A solution of (3-amino-6-chloro-2-fluorophenyl)methanol (818 mg, 4.7 mmol, 1 equiv), 3-methyl-1-(oxan-2-yl)pyrazolo[3,4-b]pyridin-5-ol (1.6 g, 7 mmol, 1.5 equiv) and PPh₃ (1.8 g, 7 mmol, 1.5 equiv) in DCM (10 mL) was treated with TMAD (1.2 g, 7 mmol, 1.5 equiv), then stirred for 1 h. The resulting mixture was diluted with EA (50 mL), and the organics were washed with water and brine, dried over anhydrous Na₂SO₄, then concentrated. The residue was purified by silica gel column chromatography, eluting with PE/EA (1/1) to afford 4-chloro-2-fluoro-3-([[3-methyl-1-(oxan-2-yl)pyrazolo[3,4-b]pyridin-5-yl]oxy]methyl)aniline (916 mg, 50% yield) as a light yellow solid.

LCMS (ES, m/z): [M+H]⁺: 391.

### Synthesis of 77-d: N-(4-chloro-2-fluoro-3-(((3-methyl-1-(tetrahydro-2H-pyran-2-yl)-1H-pyrazolo[3,4-b]pyridin-5-yl)oxy)methyl)phenyl)-5-fluoro-2-methylpyridine-3-sulfonamide

To a stirred solution of 4-chloro-2-fluoro-3-([[3-methyl-1-(oxan-2-yl)pyrazolo[3,4-b]pyridin-5-yl]oxy]methyl)aniline (250 mg, 0.64 mmol, 1 equiv) in pyridine (2.5 mL) was added 5-fluoro-2-methylpyridine-3-sulfonyl chloride (268 mg, 1.3 mmol, 2 equiv). The reaction was stirred for 1 h then diluted with water (2 mL). The resulting mixture was extracted with DCM (3×10 mL). The combined organics were washed with water (2×10 mL) and brine (10 mL), dried over anhydrous Na₂SO₄ then concentrated under reduced pressure. The residue was purified by silica gel column chromatography, eluting with 0-50% EtOAc/PE to afford N-[4-chloro-2-fluoro-3-([[3-methyl-1-(oxan-2-yl)pyrazolo[3,4-b]pyridin-5-yl]oxy]methyl)phenyl]-5-fluoro-2-methylpyridine-3-sulfonamide (163 mg, 45% yield) as a yellow solid.

LCMS (ES, m/z): [M+H]⁺:564.

### Synthesis of Compound 77: N-(4-chloro-2-fluoro-3-(((3-methyl-1H-pyrazolo[3,4-b]pyridin-5-yl)oxy)methyl)phenyl)-5-fluoro-2-methylpyridine-3-sulfonamide

N-[4-chloro-2-fluoro-3-([[3-methyl-1-(oxan-2-yl)pyrazolo[3,4-b]pyridin-5-yl]oxy]methyl)phenyl]-5-fluoro-2-methylpyridine-3-sulfonamide (153 mg, 0.27 mmol, 1 equiv) was treated with 4 N HCl in MeOH (4 mL) and stirred for 1 h at room temperature. The resulting mixture was concentrated and the residue purified by Prep-HPLC with the following conditions: Column: Sunfire Prep C18 OBD Column, 50*250 mm 5 μm 10 nm; Mobile Phase: 5-45% MeCN/0.05% aqueous ammonia; to afford N-[4-chloro-2-fluoro-3-[([3-methyl-1H-pyrazolo[3,4-b]pyridin-5-yl]oxy)methyl]phenyl]-5-fluoro-2-methylpyridine-3-sulfonamide (66 mg, 51% yield) as a white solid.

LCMS (ES, m/z): [M+H]⁺: 480
¹H NMR (300 MHz, DMSO-d₆) δ 13.09 (s, 1H), 10.93 (s, 1H), 8.67 (d, J=2.8 Hz, 1H), 8.19 (d, J=2.7 Hz, 1H), 7.94 (dd, J=8.3, 2.9 Hz, 1H), 7.87 (d, J=2.7 Hz, 1H), 7.34 (d, J=6.0 Hz, 2H), 5.14 (d, J=2.1 Hz, 2H), 2.76 (s, 3H), 2.48 (s, 3H).

### Example 89: Synthesis of N-(4-chloro-2-fluoro-3-(((3-methyl-1H-pyrazolo[3,4-b]pyridin-5-yl)oxy)methyl)phenyl)-5-fluoro-2-methoxypyridine-3-sulfonamide (Compound 78)

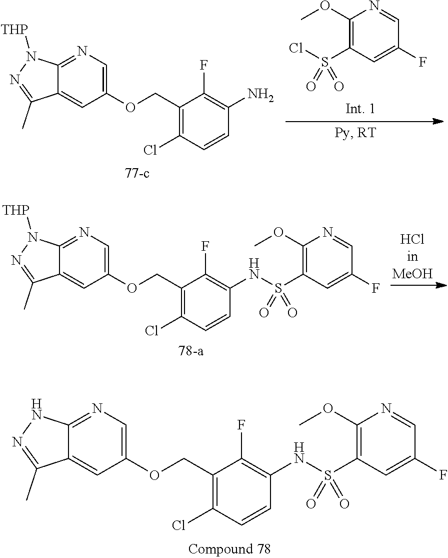

### Synthesis of 78-a: ethyl N-(4-chloro-2-fluoro-3-(((3-methyl-1-(tetrahydro-2H-pyran-2-yl)-1H-pyrazolo[3,4-b]pyridin-5-yl)oxy)methyl)phenyl)-5-fluoro-2-methoxypyridine-3-sulfonamide

To a stirred solution of 4-chloro-2-fluoro-3-([[3-methyl-1-(oxan-2-yl)pyrazolo[3,4-b]pyridin-5-yl]oxy]methyl)aniline (250 mg, 0.64 mmol, 1 equiv) in pyridine (2.5 mL) was added 5-fluoro-2-methoxypyridine-3-sulfonyl chloride (288 mg, 1.28 mmol, 2 equiv) in portions at room temperature. The resulting mixture was stirred 1 h at room temperature. The resulting mixture was diluted with water (2 mL). The resulting mixture was extracted with DCM (3×10 mL). The combined organic layers were washed with water (2×10 mL) and brine (10 mL), dried over anhydrous Na₂SO₄. After filtration, the filtrate was concentrated under reduced pressure. The residue was purified by silica gel column chromatography, eluted with EtOAc/PE (0-50%) to afford N-[4-chloro-2-fluoro-3-([[3-methyl-1-(oxan-2-yl)pyrazolo[3,4-b]pyridin-5-yl]oxy]methyl)phenyl]-5-fluoro-2-methoxypyridine-3-sulfonamide (186 mg, 50%) as a yellow solid.

LCMS (ES, m/z): [M+H]⁺: 580.

### Synthesis of Compound 78: N-(4-chloro-2-fluoro-3-(((3-methyl-1H-pyrazolo[3,4-b]pyridin-5-yl)oxy)methyl)phenyl)-5-fluoro-2-methoxypyridine-3-sulfonamide

A solution of N-[4-chloro-2-fluoro-3-([[3-methyl-1-(oxan-2-yl)pyrazolo[3,4-b]pyridin-5-yl]oxy]methyl)phenyl]-5-fluoro-2-methoxypyridine-3-sulfonamide (176 mg, 0.3 mmol, 1 equiv) in 4M HCl in MeOH (4 mL) was stirred for 1 h at room temperature. The resulting mixture was concentrated under vacuum. The crude product was purified by Prep-HPLC with the following conditions (Column: Sunfire Prep C₁₈ OBD Column, 50*250 mm, 5 μm, 10 nm; Mobile Phase A:Water (0.05% NH₃H₂O), Mobile Phase B:ACN; Flow rate: 90 mL/min; Gradient: 5 B to 45 B in 15 min;) to afford N-[4-chloro-2-fluoro-3-[([3-methyl-1H-pyrazolo[3,4-b]pyridin-5-yl]oxy)methyl]phenyl]-5-fluoro-2-methoxypyridine-3-sulfonamide (78 mg, 52%) as a light yellow solid.

LCMS (ES, m/z): [M+H]⁺: 496.
¹H NMR (300 MHz, DMSO-d₆) δ 13.09 (s, 1H), 10.09 (br, 1H) 8.42 (s, 1H), 8.20 (s, 1H), 8.02-7.99 (m, 1H), 7.89-7.88 (m, 1H), 7.04-7.31 (m, 2H), 5.16 (s, 2H), 3.88 (s, 3H), 2.46 (s, 3H).

### Example 90: Synthesis of 5-chloro-N-[3-chloro-5-fluoro-4-[([3-methyl-1H-pyrazolo[3,4-b]pyridin-5-yl]oxy)methyl]pyridin-2-yl]-2-methoxypyridine-3-sulfonamide (Compound 79)

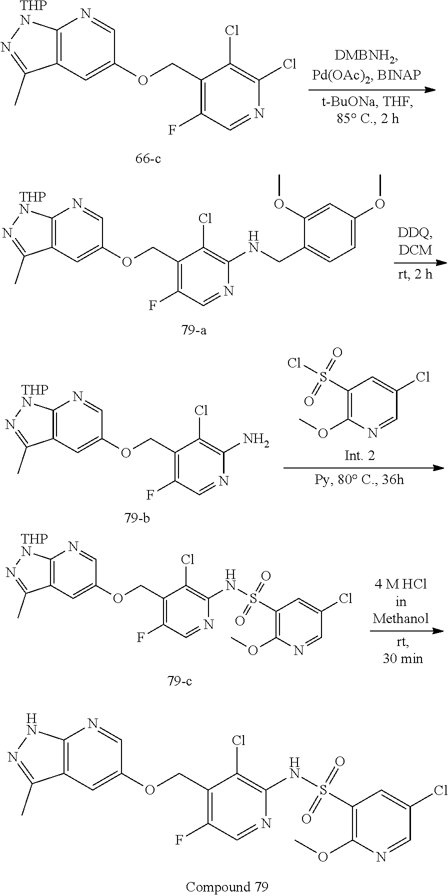

### Synthesis of 79-a: 3-chloro-N-[(2,4-dimethoxyphenyl)methyl]-5-fluoro-4-([[3-methyl-1-(oxan-2-yl)pyrazolo[3,4-b]pyridin-5-yl]oxy]methyl)pyridin-2-amine

To a stirred solution of 2,3-dichloro-5-fluoro-4-([[3-methyl-1-(oxan-2-yl)pyrazolo[3,4-b]pyridin-5-yl]oxy]methyl)pyridine (500 mg, 1.2 mmol, 1 equiv) in THF (30 mL) was added 1-(3,4-dimethylphenyl)methanamine (246 mg, 1.8 mmol, 1.5 equiv), Pd(OAc)₂ (55 mg, 0.2 mmol, 0.2 equiv), BINAP (152 mg, 0.2 mmol, 0.2 equiv) and t-BuONa (234 mg, 2.4 mmol, 2 equiv). The reaction was stirred for 2 h at 85° C. under nitrogen atmosphere, then cooled and diluted with H₂O (10 mL). The resulting mixture was extracted with ethyl acetate (4×10 mL). The combined organics were washed with brine (10 mL), dried over anhydrous sodium sulfate and concentrated. The residue was purified by Flash-Prep-HPLC with the following conditions: Column, WelFlash TM C18-I, Spherical C18 20-m, 120 g; mobile phase: 40-80% MeCN/0.1% aqueous formic acid; to afford 3-chloro-N-[(2,4-dimethoxyphenyl)methyl]-5-fluoro-4-([[3-methyl-1-(oxan-2-yl)pyrazolo[3,4-b]pyridin-5-yl]oxy]methyl)pyridin-2-amine (362 mg, 55% yield) as a white solid.

LCMS (ES, m/z): [M+H]⁺: 542

### Synthesis of 79-b: 3-chloro-5-fluoro-4-([[3-methyl-1-(oxan-2-yl)pyrazolo[3,4-b]pyridin-5-yl]oxy]methyl)pyridin-2-amine

To a stirred solution of 3-chloro-N-[(2,4-dimethoxyphenyl)methyl]-5-fluoro-4-([[3-methyl-1-(oxan-2-yl)pyrazolo[3,4-b]pyridin-5-yl]oxy]methyl)pyridin-2-amine (100 mg, 0.2 mmol, 1 equiv) in DCM (4 mL) was added DDQ (84 mg, 0.4 mmol, 2 equiv). The resulting solution was stirred for 2 h then concentrated. The residue was purified by Flash-Prep-HPLC with the following conditions: Column, WelFlash TM C18-I, Spherical C18 20-40 μm, 120 g; mobile phase: 30-80% MeCN/0.1% aqueous formic acid; to afford 3-chloro-5-fluoro-4-([[3-methyl-1-(oxan-2-yl)pyrazolo[3,4-b]pyridin-5-yl]oxy]methyl)pyridin-2-amine (50 mg, 69% yield) as a brown solid.

LCMS (ES, m/z): [M+H]⁺: 392

### Synthesis of 79-c: 5-chloro-N-[3-chloro-5-fluoro-4-([[3-methyl-1-(oxan-2-yl)pyrazolo[3,4-b]pyridin-5-yl]oxy]methyl)pyridin-2-yl]-2-methoxypyridine-3-sulfonamide

3-Chloro-5-fluoro-4-([[3-methyl-1-(oxan-2-yl)pyrazolo[3,4-b]pyridin-5-yl]oxy]methyl)pyridin-2-amine (40 mg, 0.1 mmol, 1 equiv) in pyridine (2 mL) was treated with 5-chloro-2-methoxypyridine-3-sulfonyl chloride (74 mg, 0.3 mmol, 3 equiv). The resulting solution was stirred for 36 h at 80° C. then concentrated. The residue was purified by Flash-Prep-HPLC with the following conditions: Column, WelFlash TM C18-I, Spherical C18 20-40 μm, 120 g; mobile phase: 50-80% MeCN/0.1% aqueous formic acid; to afford 5-chloro-N-[3-chloro-5-fluoro-4-([[3-methyl-1-(oxan-2-yl)pyrazolo[3,4-b]pyridin-5-yl]oxy]methyl)pyridin-2-yl]-2-methoxypyridine-3-sulfonamide (6 mg, 10% yield) as a white solid.

LCMS (ES, m/z): [M+H]⁺: 597

### Synthesis of Compound 79: 5-chloro-N-[3-chloro-5-fluoro-4-[([3-methyl-1H-pyrazolo[3,4-b]pyridin-5-yl]oxy)methyl]pyridin-2-yl]-2-methoxypyridine-3-sulfonamide

5-Chloro-N-[3-chloro-5-fluoro-4-([[3-methyl-1-(oxan-2-yl)pyrazolo[3,4-b]pyridin-5-yl]oxy]methyl)pyridin-2-yl]-2-methoxypyridine-3-sulfonamide (6 mg, 0.01 mmol, 1 equiv) was treated with 4 M HCl in MeOH (1 mL) at room temperature. The resulting solution was stirred for 30 min then concentrated. The residue was purified by prep-HPLC with the following conditions: Column, welch Vltimate XB—C18, 50×250 mm, 10 μm; Mobile Phase: 38-58% MeCN/0.1% aqueous formic acid; to afford 5-chloro-N-[3-chloro-5-fluoro-4-[([3-methyl-1H-pyrazolo[3,4-b]pyridin-5-yl]oxy)methyl]pyridin-2-yl]-2-methoxypyridine-3-sulfonamide (2 mg, 39% yield) as a white solid.

LCMS (ES, m/z): [M+H]⁺: 513
¹H NMR (300 MHz, Methanol-d₄) δ 8.29 (m, 3H), 8.03 (s, 1H), 7.87 (d, J=2.7 Hz, 1H), 5.33 (d, J=1.7 Hz, 2H), 3.99 (s, 3H), 2.57 (s, 3H).

### Example 91: Synthesis of N-[2,4-difluoro-3-([[3-(2,2,2-trifluoroethyl)-1H-pyrazolo[3,4-b]pyridin-5-yl]oxy]methyl)phenyl]-5-fluoro-2-methoxypyridine-3-sulfonamide (Compound 80)

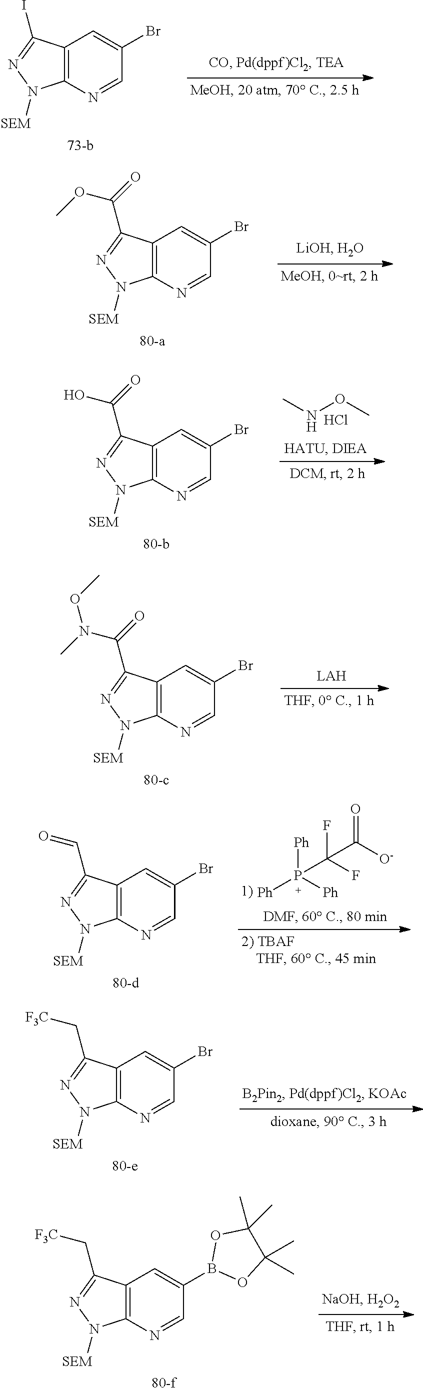

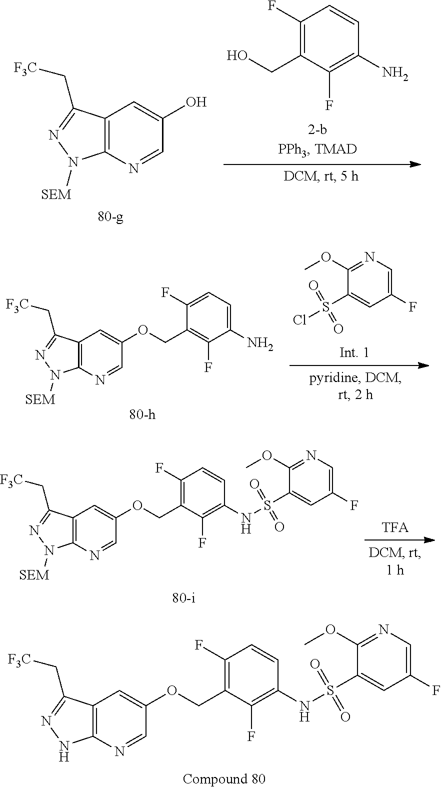 [SMILES: C[Si](C)(C)CCOCn1nc(CC(F)(F)F)c2cc(O)cnc21 ; Nc1ccc(F)c(CO)c1F ; C[Si](C)(C)CCOCn1nc(CC(F)(F)F)c2cc(OCc3c(F)ccc(N)c3F)cnc21 ; COc1ncc(F)cc1S(=O)(=O)Cl ; COc1ncc(F)cc1S(=O)(=O)Nc1ccc(F)c(COc2cnc3c(c2)c(CC(F)(F)F)nn3COCC[Si](C)(C)C)c1F ; COc1ncc(F)cc1S(=O)(=O)Nc1ccc(F)c(COc2cnc3[nH]nc(CC(F)(F)F)c3c2)c1F]

### Synthesis of 80-a: methyl 5-bromo-1-[[2-(trimethylsilyl)ethoxy]methyl]pyrazolo[3,4-b]pyridine-3-carboxylate

To a stirred solution of 5-bromo-3-iodo-1-[[2-(trimethylsilyl)ethoxy]methyl]pyrazolo[3,4-b]pyridine 5-bromo-3-iodo-1-[[2-(trimethylsilyl)ethoxy]methyl]pyrazolo[3,4-b]pyridine (3 g, 6.6 mmol, 1 equiv) and TEA (2 g, 20 mmol, 3 equiv) in MeOH (30 mL) was added Pd(dppf)Cl₂ (0.48 g, 0.66 mmol, 0.1 equiv) in portions. The reaction was stirred for 2.5 h at 70° C. under 20 atm CO pressure, then concentrated. The residue was purified by silica gel column chromatography, eluting with PE:EA (20:1) to afford methyl 5-bromo-1-[[2-(trimethylsilyl)ethoxy]methyl]pyrazolo[3,4-b]pyridine-3-carboxylate (2.4 g, 94% yield) as an oil.

LCMS (ES, m/z): [M+H]⁺: 386

### Synthesis of 80-b: 5-bromo-1-((2-(trimethylsilyl)ethoxy)methyl)-1H-pyrazolo[3,4-b]pyridine-3-carboxylic acid

To a stirred solution of methyl 5-bromo-1-[[2-(trimethylsilyl)ethoxy]methyl]pyrazolo[3,4-b]pyridine-3-carboxylate (2.2 g, 5.8 mmol, 1 equiv) in MeOH (20 mL) was added LiOH·H₂O (1.2 g, 29 mmol, 5 equiv) in H₂O (2 mL) dropwise at 0° C. The resulting mixture was stirred for 2 h then diluted with H₂O (40 mL) and EA (20 mL). The mixture was acidified to pH 5 with AcOH, and extracted with EA (3×20 mL). The combined organics were washed with brine (40 mL), dried over anhydrous Na₂SO₄ and concentrated to afford crude 5-bromo-1-((2-trimethylsilyl)ethoxy)methyl)-1H-pyrazolo[3,4-b]pyridine-3-carboxylic acid (2.1 g, 97% yield) as a white solid which was used in next step directly without further purification.

LCMS (ES, m/z): [M+H]⁺: 372

### Synthesis of 80-c: 5-bromo-N-methoxy-N-methyl-1-[[2-(trimethylsilyl)ethoxy]methyl]pyrazolo[3,4-b]pyridine-3-carboxamide

To a stirred solution of 5-bromo-1-[[2-(trimethylsilyl)ethoxy]methyl]pyrazolo[3,4-b]pyridine-3-carboxylic acid (2.2 g, 5.9 mmol, 1 equiv) and N,O-dimethylhydroxylamine (0.39 g, 6.4 mmol, 1.1 equiv) in DCM (40 mL) were added HATU (2.7 g, 7 mmol, 1.2 equiv) and DIEA (2.3 g, 17.6 mmol, 3 equiv). The resulting mixture was stirred for 2 h then diluted with H₂O (50 mL) and extracted with DCM (3×20 mL). The combined organics were washed with brine (50 mL), dried over anhydrous Na₂SO₄ and concentrated. The residue was purified by silica gel column chromatography, eluting with PE:EA (10:1) to afford 5-bromo-N-methoxy-N-methyl-1-[[2-(trimethylsilyl)ethoxy]methyl]pyrazolo[3,4-b]pyridine-3-carboxamide (2.24 g, 92% yield) as a light yellow oil.

LCMS (ES, m/z): [M+H]⁺: 415

### Synthesis of 80-d: 5-bromo-1-[[2-(trimethylsilyl)ethoxy]methyl]pyrazolo[3,4-b]pyridine-3-carbaldehyde

To a stirred solution of 5-bromo-N-methoxy-N-methyl-1-[[2-(trimethylsilyl)ethoxy]methyl]pyrazolo[3,4-b]pyridine-3-carboxamide (1 g, 2.4 mmol, 1 equiv) in THE (10 mL) was added LAH (0.11 g, 2.9 mmol, 1.2 equiv) in portions at 0° C. The resulting mixture was stirred for 1 h at 0° C. The reaction was quenched by the addition of sat. NaHSO₄ (aq.) (30 mL) at 0° C., then extracted with EtOAc (3×20 mL). The combined organics were washed with brine (20 mL), dried over anhydrous Na₂SO₄ and concentrated. The residue was purified by silica gel column chromatography, eluting with PE/EtOAc (20:1) to afford 5-bromo-1-[[2-(trimethylsilyl)ethoxy]methyl]pyrazolo[3,4-b]pyridine-3-carbaldehyde (703 mg, 82% yield) as a yellow oil.

LCMS (ES, m/z): [M+H]⁺: 356

### Synthesis of 80-e: 5-bromo-3-(2,2,2-trifluoroethyl)-1-[[2-(trimethylsilyl)ethoxy]methyl]pyrazolo[3,4-b]pyridine

To a stirred solution of 5-bromo-1-[[2-(trimethylsilyl)ethoxy]methyl]pyrazolo[3,4-b]pyridine-3-carbaldehyde (1.3 g, 3.7 mmol, 1 equiv) in DMF (10 mL) was added difluoro(triphenylphosphaniumyl)acetate (2.6 g, 7.3 mmol, 2 equiv) in portions at room temperature. The resulting mixture was stirred for 80 min at 60° C. The mixture was allowed to cool to room temperature and TBAF (5.6 mL, 11 mmol, 3 equiv) was added dropwise over 1 min at room temperature. The resulting mixture was stirred for additional 45 min at 60° C., then cooled and diluted with H₂O (50 mL), then extracted with EtOAc (3×10 mL). The combined organics were washed with brine (20 mL), dried over anhydrous Na₂SO₄ and concentrated. The residue was purified by Flash-Prep-HPLC with the following conditions: Column, WelFlash TM C18-I, Spherical C18 20-40 μm, 120 g; mobile phase: 50-95% MeCN/0.1% aqueous formic acid; to afford 5-bromo-3-(2,2,2-trifluoroethyl)-1-[[2-(trimethylsilyl)ethoxy]methyl]pyrazolo[3,4-b]pyridine (520 mg, 35% yield) as a yellow oil.

LCMS (ES, m/z): [M+H]⁺: 410

### Synthesis of 80-f: 5-(4,4,5,5-tetramethyl-1,3,2-dioxaborolan-2-yl)-3-(2,2,2-trifluoroethyl)-1-[[2-trimethylsilyl)ethoxy]methyl]pyrazolo[3,4-b]pyridine

To a stirred solution of 5-bromo-3-(2,2,2-trifluoroethyl)-1-[[2-(trimethylsilyl)ethoxy]methyl]pyrazolo[3,4-b]pyridine (495 mg, 1.2 mmol, 1 equiv) and bis(pinacolato)diboron (337 mg, 1.3 mmol, 1.1 equiv) in dioxane (6 mL) were added KOAc (237 mg, 2.4 mmol, 2 equiv) and Pd(dppf)Cl₂ (88 mg, 0.12 mmol, 0.1 equiv) in portions, at room temperature under nitrogen atmosphere. The resulting mixture was stirred for 3 h at 90° C. then concentrated to afford crude 5-(4,4,5,5-tetramethyl-1,3,2-dioxaborolan-2-yl)-3-(2,2,2-trifluoroethyl)-1-[[2-(trimethylsilyl)ethoxy]methyl]pyrazolo[3,4-b]pyridine (550 mg) as a brown oil which was used in next step directly without further purification.

LCMS (ES, m/z): [M+H]⁺: 458

### Synthesis of 80-g: 3-(2,2,2-trifluoroethyl)-1-[[2-(trimethylsilyl)ethoxy]methyl]pyrazolo[3,4-b]pyridin-5-ol

To 5-(4,4,5,5-tetramethyl-1,3,2-dioxaborolan-2-yl)-3-(2,2,2-trifluoroethyl)-1-[[2-(trimethylsilyl)ethoxy]methyl]pyrazolo[3,4-b]pyridine (495 mg, 1.1 mmol, 1 equiv) in THE (5 mL) was added 2% aqueous NaOH solution (5 mL) and 30% H₂O₂ (368 mg, 10.82 mmol, 10 equiv). The reaction was stirred for 1 h then concentrated. The residue was purified by Flash-Prep-HPLC with the following conditions: Column, WelFlash TM C18-I, Spherical C18 20-40 μm, 120 g; mobile phase: 30-75% MeCN/0.1% aqueous formic acid; to afford 3-(2,2,2-trifluoroethyl)-1-[[2-(trimethylsilyl)ethoxy]methyl]pyrazolo[3,4-b]pyridin-5-ol (207 mg, 55% yield) as a brown oil.

LCMS (ES, m/z): [M+H]⁺: 348

### Synthesis of 80-h: 2,4-difluoro-3-([[3-(2,2,2-trifluoroethyl)-1-[[2-(trimethylsilyl)ethoxy]methyl]pyrazolo[3,4-b]pyridin-5-yl]oxy]methyl)aniline

To a stirred solution of 3-(2,2,2-trifluoroethyl)-1-[[2-(trimethylsilyl)ethoxy]methyl]pyrazolo[3,4-b]pyridin-5-ol (200 mg, 0.58 mmol, 1 equiv) and (3-amino-2,6-difluorophenyl)methanol (110 mg, 0.69 mmol, 1.2 equiv) in DCM (2 mL) were added TMAD (149 mg, 0.86 mmol, 1.5 equiv) and PPh₃ (227 mg, 0.86 mmol, 1.5 equiv). The resulting mixture was stirred for 5 h then concentrated. The residue was purified by Flash-Prep-HPLC with the following conditions: Column, WelFlash TM C18-I, Spherical C18 20-m, 120 g; mobile phase: 40-90% MeCN/0.1% aqueous formic acid; to afford 2,4-difluoro-3-([[3-(2,2,2-trifluoroethyl)-1-[[2-(trimethylsilyl)ethoxy]methyl]pyrazolo[3,4-b]pyridin-5-yl]oxy]methyl)aniline (192 mg, 68% yield) as a brown solid.

LCMS (ES, m/z): [M+H]⁺: 489

### Synthesis of 80-i: N-[2,4-difluoro-3-([[3-(2,2,2-trifluoroethyl)-1-[[2-(trimethylsilyl)ethoxy]methyl]pyrazolo[3,4-b]pyridin-5-yl]oxy]methyl)phenyl]-5-fluoro-2-methoxypyridine-3-sulfonamide

To a stirred solution of 2,4-difluoro-3-([[3-(2,2,2-trifluoroethyl)-1-[[2-(trimethylsilyl)ethoxy]methyl]pyrazolo[3,4-b]pyridin-5-yl]oxy]methyl)aniline (187 mg, 0.38 mmol, 1 equiv) in pyridine (4 mL) was added 5-fluoro-2-methoxypyridine-3-sulfonyl chloride (130 mg, 0.57 mmol, 1.5 equiv) in DCM (0.5 mL). The resulting mixture was stirred for 2 h then concentrated. The residue was purified by Flash-Prep-HPLC with the following conditions: Column, WelFlash TM C18-I, Spherical C18 20-40 μm, 120 g; mobile phase: 50-95% MeCN/0.1% aqueous formic acid; to afford N-[2,4-difluoro-3-([[3-(2,2,2-trifluoroethyl)-1-[[2-(trimethylsilyl)ethoxy]methyl]pyrazolo[3,4-b]pyridin-5-yl]oxy]methyl)phenyl]-5-fluoro-2-methoxypyridine-3-sulfonamide (249 mg, 96% yield) as a brown oil.

LCMS (ES, m/z): [M+H]⁺: 678

### Synthesis of Compound 80: N-[2,4-difluoro-3-([[3-(2,2,2-trifluoroethyl)-1H-pyrazolo[3,4-b]pyridin-5-yl]oxy]methyl)phenyl]-5-fluoro-2-methoxypyridine-3-sulfonamide

A solution of N-[2,4-difluoro-3-([[3-(2,2,2-trifluoroethyl)-1-[[2-(trimethylsilyl)ethoxy]methyl]pyrazolo[3,4-b]pyridin-5-yl]oxy]methyl)phenyl]-5-fluoro-2-methoxypyridine-3-sulfonamide (256 mg, 0.38 mmol, 1 equiv) in TFA (1 mL) and DCM (3 mL) was stirred for 1 h then concentrated. The residue was purified by prep-HPLC with the following conditions: Column, welch Vltimate XB—C18, 50×250 mm, 10 μm; Mobile Phase: 30-60% MeCN/0.1% aqueous formic acid; to afford N-[2,4-difluoro-3-([[3-(2,2,2-trifluoroethyl)-1H-pyrazolo[3,4-b]pyridin-5-yl]oxy]methyl)phenyl]-5-fluoro-2-methoxypyridine-3-sulfonamide (92 mg, 44% yield) as a white solid.

LCMS (ES, m/z): [M+H]⁺: 548
¹H NMR (300 MHz, DMSO-d₆) δ 13.66 (s, 1H), 10.37 (s, 1H), 8.45 (d, J=3.0 Hz, 1H), 8.26 (d, J=2.8 Hz, 1H), 8.02-7.90 (m, 2H), 7.38 (td, J=8.9, 5.9 Hz, 1H), 7.22-7.10 (m, 1H), 5.13 (s, 2H), 4.05 (d, J=11.3 Hz, 1H), 3.98 (d, J=11.6 Hz, 1H), 3.91 (s, 3H).

### Example 92: Synthesis of N-[3-([[3-(1,1-difluoroethyl)-1H-pyrazolo[3,4-b]pyridin-5-yl]oxy]methyl)-2,4-difluorophenyl]-5-fluoro-2-methoxypyridine-3-sulfonamide (Compound 81)

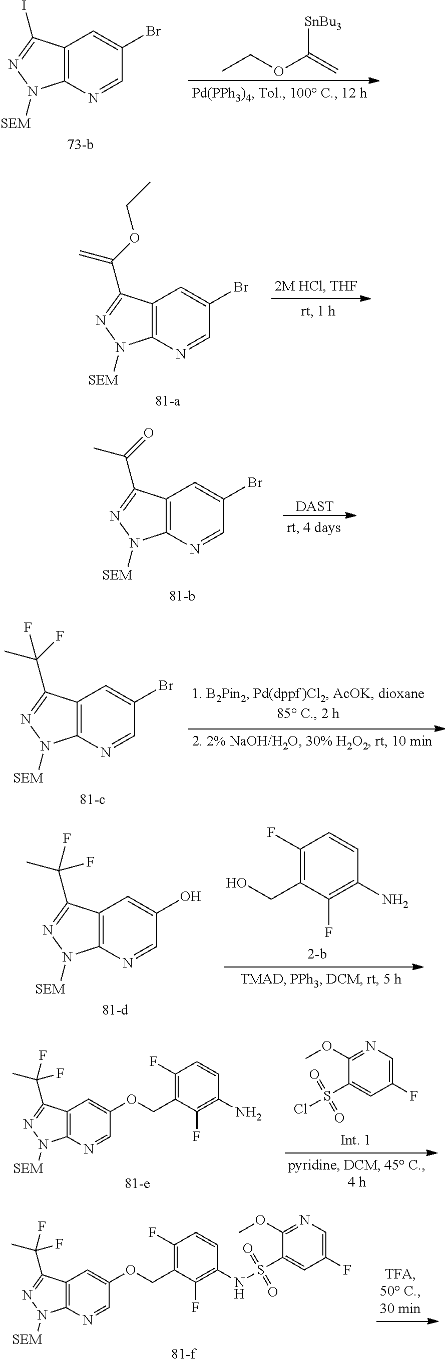

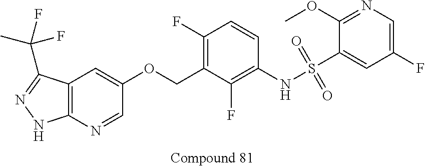 [SMILES: COc1ncc(F)cc1S(=O)(=O)Nc1ccc(F)c(COc2cnc3[nH]nc(C(C)(F)F)c3c2)c1F]

### Synthesis of 81-a: 5-bromo-3-(1-ethoxyethenyl)-1-[[2-(trimethylsilyl)ethoxy]methyl]pyrazolo[3,4-b]pyridine

To 5-bromo-3-iodo-1-[[2-(trimethylsilyl)ethoxy]methyl]pyrazolo[3,4-b]pyridine (400 mg, 0.88 mmol, 1 equiv) in toluene (10 mL) was added tributyl(1-ethoxyethenyl)stannane (318 mg, 0.88 mmol, 1 equiv) and Pd(PPh₃)₄ (1 mg, 0.044 mmol, 0.05 equiv). The resulting solution was stirred for 12 h at 100° C. then concentrated. The residue was applied onto a silica gel column, eluting with ethyl acetate/petroleum ether (1:10) to give 5-bromo-3-(1-ethoxyethenyl)-1-[[2-(trimethylsilyl)ethoxy]methyl]pyrazolo[3,4-b]pyridine (300 mg, 77% yield) as a yellow oil.

LCMS (ES, m/z): [M+H]⁺: 398

### Synthesis of 81-b: 1-(5-bromo-1-[[2-(trimethylsilyl)ethoxy]methyl]pyrazolo[3,4-b]pyridin-3-yl)ethanone

5-Bromo-3-(1-ethoxyethenyl)-1-[[2-(trimethylsilyl)ethoxy]methyl]pyrazolo[3,4-b]pyridine (300 mg, 0.75 mmol, 1 equiv) in THE (5 mL) was treated with 2 M HCl (2 mL). The resulting solution was stirred for 1 h, then the pH was adjusted to 7-8 with NaHCO₃/H₂O·H₂O (20 ml) and the mixture extracted with dichloromethane (2×20 mL). The combined organics were washed with brine (30 ml), dried over anhydrous sodium sulfate and concentrated to give 1-(5-bromo-1-[[2-(trimethylsilyl)ethoxy]methyl]pyrazolo[3,4-b]pyridin-3-yl)ethanone (260 mg, 84% yield) as a yellow oil.

LCMS (ES, m/z): [M+H]⁺: 370

### Synthesis of 81-c: 5-bromo-3-(1,1-difluoroethyl)-1-[[2-(trimethylsilyl)ethoxy]methyl]pyrazolo[3,4-b]pyridine

1-(5-Bromo-1-[[2-(trimethylsilyl)ethoxy]methyl]pyrazolo[3,4-b]pyridin-3-yl)ethanone (500 mg, 1.4 mmol, 1 equiv) was treated with DAST (3 mL) and the solution was stirred for 4 days at room temperature. The reaction was quenched with water/ice (50 mL), and the pH adjusted to 7-8 with NaHCO₃/H₂O. The resulting solution was extracted with dichloromethane (3×20 mL), and the combined organic layers were dried over anhydrous sodium sulfate then concentrated. The residue was applied to a silica gel column, eluting with ethyl acetate/petroleum ether (1:10). 5-Bromo-3-(1,1-difluoroethyl)-1-[[2-(trimethylsilyl)ethoxy]methyl]pyrazolo[3,4-b]pyridine (120 mg, 22% yield) was obtained as a colorless oil.

LCMS (ES, m/z): [M+H]⁺: 392.

### Synthesis of 81-d: 3-(1,1-difluoroethyl)-1-[[2-(trimethylsilyl)ethoxy]methyl]pyrazolo[3,4-b]pyridin-5-ol

5-Bromo-3-(1,1-difluoroethyl)-1-[[2-(trimethylsilyl)ethoxy]methyl]pyrazolo[3,4-b]pyridine (120 mg, 0.31 mmol, 1 equiv) in dioxane (5 mL) was treated with Pd(dppf)Cl₂ (45 mg, 0.062 mmol, 0.2 equiv), KOAc (90 mg, 0.92 mmol, 3 equiv) and B₂Pin₂ (117 mg, 0.46 mmol, 1.5 equiv). The solution was stirred for 2 h at 85° C. then cooled to room temperature. 2% aqueous NaOH solution (10 mL) was added, followed by 30% H₂O₂ (1 mL). The resulting solution was stirred for 10 min at room temperature, then diluted with H₂O (30 mL). The mixture was extracted with dichloromethane (3×20 mL) and the combined organics were dried over anhydrous sodium sulfate, then concentrated. The residue was applied to a silica gel column, eluting with ethyl acetate/PE (1:4) to give 3-(1,1-difluoroethyl)-1-[[2-(trimethylsilyl)ethoxy]methyl]pyrazolo[3,4-b]pyridin-5-ol (80 mg) as a colorless oil.

LCMS (ES, m/z): [M+H]⁺: 330

### Synthesis of 81-e: 3-([[3-(1,1-difluoroethyl)-1-[[2-(trimethylsilyl)ethoxy]methyl]pyrazolo[3,4-b]pyridin-5-yl]oxy]methyl)-2,4-difluoroaniline

To 3-(1,1-difluoroethyl)-1-[[2-(trimethylsilyl)ethoxy]methyl]pyrazolo[3,4-b]pyridin-5-ol (80 mg, 0.24 mmol, 1 equiv) in DCM (5 mL) was added (3-amino-2,6-difluorophenyl)methanol (58 mg, 0.36 mmol, 1.5 equiv), TMAD (63 mg, 0.36 mmol, 1.5 equiv) and PPh₃ (96 mg, 0.36 mmol, 1.5 equiv). The reaction was stirred for 5 h then concentrated. The crude product was purified by Flash-Prep-HPLC with the following conditions: Column, C₁₈ silica gel; mobile phase: 20% MeCN/H₂O; to afford 3-([[3-(1,1-difluoroethyl)-1-[[2-(trimethylsilyl)ethoxy]methyl]pyrazolo[3,4-b]pyridin-5-yl]oxy]methyl)-2,4-difluoroaniline (80 mg, 63% yield) as a colorless oil.

LCMS (ES, m/z): [M+H]⁺: 471

### Synthesis of 81-f: N-[3-([[3-(1,1-difluoroethyl)-1-[[2-(trimethylsilyl)ethoxy]methyl]pyrazolo[3,4-b]pyridin-5-yl]oxy]methyl)-2,4-difluorophenyl]-5-fluoro-2-methoxypyridine-3-sulfonamide

3-([[3-(1,1-Difluoroethyl)-1-[[2-(trimethylsilyl)ethoxy]methyl]pyrazolo[3,4-b]pyridin-5-yl]oxy]methyl)-2,4-difluoroaniline (80 mg, 0.17 mmol, 1 equiv) in DCM (3 mL) was treated with pyridine (0.5 mL) and 5-fluoro-2-methoxypyridine-3-sulfonyl chloride (57.5 mg, 0.26 mmol, 1.5 equiv). The resulting solution was stirred for 4 h at 45° C. then concentrated. The residue was applied to a silica gel column, eluting with ethyl acetate/petroleum ether (1:4) to give N-[3-([[3-(1,1-difluoroethyl)-1-[[2-(trimethylsilyl)ethoxy]methyl]pyrazolo[3,4-b]pyridin-5-yl]oxy]methyl)-2,4-difluorophenyl]-5-fluoro-2-methoxypyridine-3-sulfonamide (90 mg, 72% yield) as a brown oil.

LCMS (ES, m/z): [M+H]⁺: 660

### Synthesis of Compound 81: N-[3-([[3-(1,1-difluoroethyl)-1H-pyrazolo[3,4-b]pyridin-5-yl]oxy]methyl)-2,4-difluorophenyl]-5-fluoro-2-methoxypyridine-3-sulfonamide

N-[3-([[3-(1,1-Difluoroethyl)-1-[[2-(trimethylsilyl)ethoxy]methyl]pyrazolo[3,4-b]pyridin-5-yl]oxy]methyl)-2,4-difluorophenyl]-5-fluoro-2-methoxypyridine-3-sulfonamide (90 mg, 0.14 mmol, 1 equiv) in DCM (2 mL) was treated with trifluoroacetic acid (2 mL). The resulting solution was stirred for 30 min at 50° C. then concentrated. The residue was applied to a silica gel column, eluting with ethyl acetate/petroleum ether (1:1) to afford N-[3-([[3-(1,1-difluoroethyl)-1H-pyrazolo[3,4-b]pyridin-5-yl]oxy]methyl)-2,4-difluorophenyl]-5-fluoro-2-methoxypyridine-3-sulfonamide (21 mg, 29% yield) as a white solid.

LCMS (ES, m/z): [M+H]⁺: 530
¹H NMR (300 MHz, DMSO-d₆) δ 14.03 (s, 1H), 10.37 (s, 1H), 8.43 (d, J=3.0 Hz, 1H), 8.32 (d, J=2.7 Hz, 1H), 7.97 (dd, J=7.3, 3.0 Hz, 1H), 7.82 (d, J=2.8 Hz, 1H), 7.45-7.31 (m, 1H), 7.17 (t, J=9.2 Hz, 1H), 5.20 (s, 2H), 3.90 (s, 3H), 2.17 (t, J=18.9 Hz, 3H).

### Example 93: Synthesis of N-[2,4-difluoro-3-[([5-methylimidazo[1,5-b]pyridazin-3-yl]oxy)methyl]phenyl]-5-fluoro-2-methoxypyridine-3-sulfonamide (Compound 82)

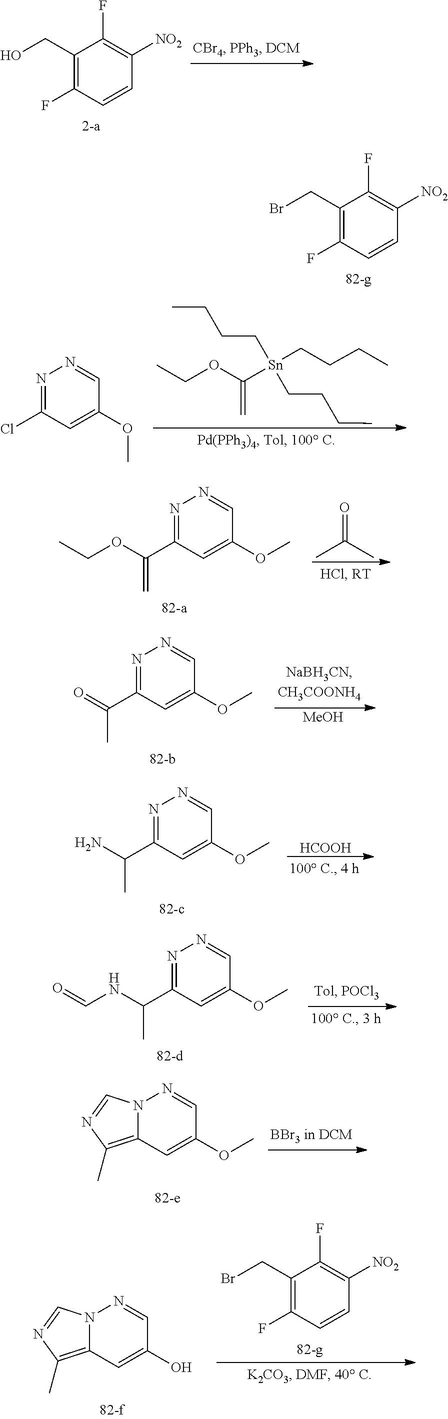

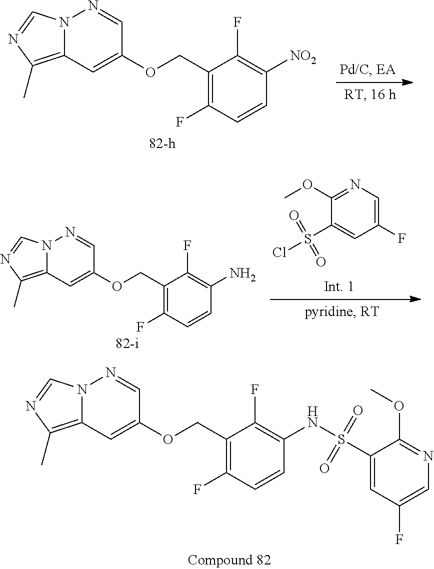 [SMILES: Cc1ncn2ncc(OCc3c(F)ccc([N+](=O)[O-])c3F)cc12 ; Cc1ncn2ncc(OCc3c(F)ccc(N)c3F)cc12 ; COc1ncc(F)cc1S(=O)(=O)Cl ; COc1ncc(F)cc1S(=O)(=O)Nc1ccc(F)c(COc2cnn3cnc(C)c3c2)c1F]

### Synthesis of 82-a: 3-(1-ethoxyvinyl)-5-methoxypyridazine

To a solution of 3-chloro-5-methoxypyridazine (5 g, 35 mmol, 1 equiv) and ethyl tributylstannanecarboxylate (18.8 g, 52 mmol, 1.5 equiv) in toluene (50 mL) was added Pd(PPh₃)₄ (4 g, 3.5 mmol, 0.1 equiv) under nitrogen atmosphere. The resulting mixture was stirred for 16 h at 110° C. under nitrogen atmosphere, then concentrated. The residue was purified by silica gel column chromatography, eluting with EA/PE (3/2) to afford 3-(1-ethoxyvinyl)-5-methoxypyridazine (3.6 g, 57% yield) as a light yellow solid.

LCMS (ES, m/z): [M+H]⁺: 181.

### Synthesis of 82-b: 1-(5-methoxypyridazin-3-yl)ethanone

To a solution of 3-(1-ethoxyethenyl)-5-methoxypyridazine (2.4 g, 13 mmol, 1 equiv) in acetone (20 mL) was added 3 M aqueous HCl (5 mL). The mixture was stirred for 4 h at room temperature, then neutralized to pH 7 with NaHCO₃ solution. The resulting mixture was extracted with EA (3×30 mL). The combined organics were washed with water and brine, dried over anhydrous Na₂SO₄ and concentrated. The residue was purified by silica gel column chromatography, eluting with PE/EA (1/1) to afford 1-(5-methoxypyridazin-3-yl)ethanone (1.48 g, 73% yield) as a light yellow oil.

LCMS (ES, m/z): [M+H]⁺: 153.

### Synthesis of 82-c: 1-(5-methoxypyridazin-3-yl)ethan-1-amine

A mixture of 1-(5-methoxypyridazin-3-yl)ethanone (1.3 g, 8.5 mmol, 1 equiv), NH₄₀Ac (6.6 g, 85 mmol, 10 equiv) and NaBH₃CN (0.54 g, 8.5 mmol, 1 equiv) in MeOH (13 mL) was stirred for 16 h at room temperature. The resulting mixture was filtered, the filter cake was washed with MeOH (3×10 mL). The filtrate was concentrated to give crude 1-(5-methoxypyridazin-3-yl)ethanamine (1.3 g).

LCMS (ES, m/z): [M+H]⁺: 154.

### Synthesis of 82-d: N-(1-(5-methoxypyridazin-3-yl)ethyl)formamide

A solution of 1-(5-methoxypyridazin-3-yl)ethanamine (1.3 g, crude) in HCOOH (13 mL) was stirred for 4 h at 100° C. The mixture was allowed to cool and was concentrated to give crude N-[1-(5-methoxypyridazin-3-yl)ethyl]formamide (1.4 g).

LCMS (ES, m/z): [M+H]⁺:182.

### Synthesis of 82-e: 3-methoxy-5-methylimidazo[1,5-b]pyridazine

To a solution of N-[1-(5-methoxypyridazin-3-yl)ethyl]formamide (1.4 g, crude) in toluene (5 mL) was added POCl₃ (1.2 g, 7.7 mmol, 1 equiv). After stirring for 4 h at 100° C. under nitrogen atmosphere, the resulting mixture was concentrated. The residue was purified by silica gel column chromatography, eluting with PE/EA (1/1) to afford 3-methoxy-5-methylimidazo[1,5-b]pyridazine (231 mg, 16% yield over 3 steps) as a light yellow solid.

LCMS (ES, m/z): [M+H]⁺: 164.

### Synthesis of 82-f: 5-methylimidazo[1,5-b]pyridazin-3-ol

A solution of 3-methoxy-5-methylimidazo[1,5-b]pyridazine (231 mg, 1.4 mmol, 1 equiv) in DCE (2 mL) was treated with 1 M BBr₃ in DCM (8.4 mL, 6 equiv) and the solution stirred for 16 h at 60° C. The reaction was concentrated, and the residue basified to pH 8 with aq. NaHCO₃. The resulting mixture was extracted with EA (3×10 mL). The combined organics were washed with brine (20 mL), dried over anhydrous Na₂SO₄ and concentrated. The residue was purified by silica gel column chromatography, eluting with EA to afford 5-methylimidazo[1,5-b]pyridazin-3-ol (110 mg, 52% yield) as a light yellow solid.

LCMS (ES, m/z): [M+H]⁺: 150.

### Synthesis of 82-g: 2-(bromomethyl)-1,3-difluoro-4-nitrobenzene

A solution of (2,6-difluoro-3-nitrophenyl)methanol (100 mg, 0.53 mmol, 1 equiv) and PPh₃ (152 mg, 0.58 mmol, 1.1 equiv) in THE (1 mL) was treated with CBr₄ (263 mg, 0.794 mmol, 1.5 equiv) in portions at 0° C., then stirred for 1 h at room temperature. The resulting mixture was diluted with EA (20 mL), and the organics were washed with water and brine, dried over anhydrous Na₂SO₄ and concentrated. The residue was purified by silica gel column chromatography, eluting with PE/EA (5/1) to afford 2-(bromomethyl)-1,3-difluoro-4-nitrobenzene (193 mg, 72% yield) as a light yellow oil.

### Synthesis of 82-h: 3-((2,6-difluoro-3-nitrobenzyl)oxy)-5-methylimidazo[1,5-b]pyridazine

To a mixture of 5-methylimidazo[1,5-b]pyridazin-3-ol (50 mg, 0.3 mmol, 1 equiv) and K₂CO₃ (46 mg, 0.3 mmol, 1 equiv) in DMF (1 mL) was added 2-(bromomethyl)-1,3-difluoro-4-nitrobenzene (84 mg, 0.3 mmol, 1 equiv) in DMF (1 ml) dropwise. The reaction was stirred for 1 h at 40° C., then diluted with EA (30 mL). The organics were washed with water and brine, dried over anhydrous Na₂SO₄ and concentrated. The residue was purified by silica gel column chromatography, eluting with EA/PE (3/2) to afford 3-[(2,6-difluoro-3-nitrophenyl)methoxy]-5-methylimidazo[1,5-b]pyridazine (68 mg, 63% yield) as a light yellow solid.

LCMS (ES, m/z): [M+H]⁺:321.

### Synthesis of 82-i: 2,4-difluoro-3-[([5-methylimidazo[1,5-b]pyridazin-3-yl]oxy)methyl]aniline

To a solution of 3-[(2,6-difluoro-3-nitrophenyl)methoxy]-5-methylimidazo[1,5-b]pyridazine (63 mg, 0.2 mmol, 1 equiv) in EA (1 mL) was added 10% Pd/C (6 mg) under nitrogen atmosphere. The mixture was hydrogenated for 16 h under a hydrogen balloon, then filtered through a Celite pad. The filtrate was concentrated to give 2,4-difluoro-3-[([5-methylimidazo[1,5-b]pyridazin-3-yl]oxy)methyl]aniline (33 mg, 58% yield) as a light yellow solid.

LCMS (ES, m/z): [M+H]⁺:291.

### Synthesis of Compound 82: N-[2,4-difluoro-3-[([5-methylimidazo[1,5-b]pyridazin-3-yl]oxy)methyl]phenyl]-5-fluoro-2-methoxypyridine-3-sulfonamide

To 2,4-difluoro-3-[([5-methylimidazo[1,5-b]pyridazin-3-yl]oxy)methyl]aniline (30 mg, 0.1 mmol, 1 equiv) in pyridine (1 mL) was added 5-fluoro-2-methoxypyridine-3-sulfonyl chloride (117 mg, 0.5 mmol, 5 equiv). This was stirred for 0.5 h at room temperature, then diluted with EA (20 mL). The organics were washed with water and brine, dried over anhydrous Na₂SO₄ and concentrated. The crude product was purified by Prep-HPLC with the following conditions: Column: XBridge Prep C₁₈ OBD Column, 5 μm, 19*150 mm; Mobile Phase: 20-36% MeCN/10 mM aqueous NH₄HCO₃; to afford N-[2,4-difluoro-3-[([5-methylimidazo[1,5-b]pyridazin-3-yl]oxy)methyl]phenyl]-5-fluoro-2-methoxypyridine-3-sulfonamide (18 mg, 36% yield) as a yellow solid.

LCMS (ES, m/z): [M+H]⁺: 515.
¹H NMR (300 MHz, DMSO-d₆) δ 10.40 (s, 1H), 8.47-8.36 (m, 2H), 8.04-7.94 (m, 2H), 7.58 (d, J=2.9 Hz, 1H), 7.47-7.33 (m, 1H), 7.17 (t, J=9.0 Hz, 1H), 5.11 (s, 2H), 3.90 (s, 3H), 2.37 (s, 3H).

### Example 94: Synthesis of N-[2,4-difluoro-3-([[5-(trifluoromethyl)imidazo[1,5-b]pyridazin-3-yl]oxy]methyl)phenyl]-5-fluoro-2-methoxypyridine-3-sulfonamide (Compound 83)

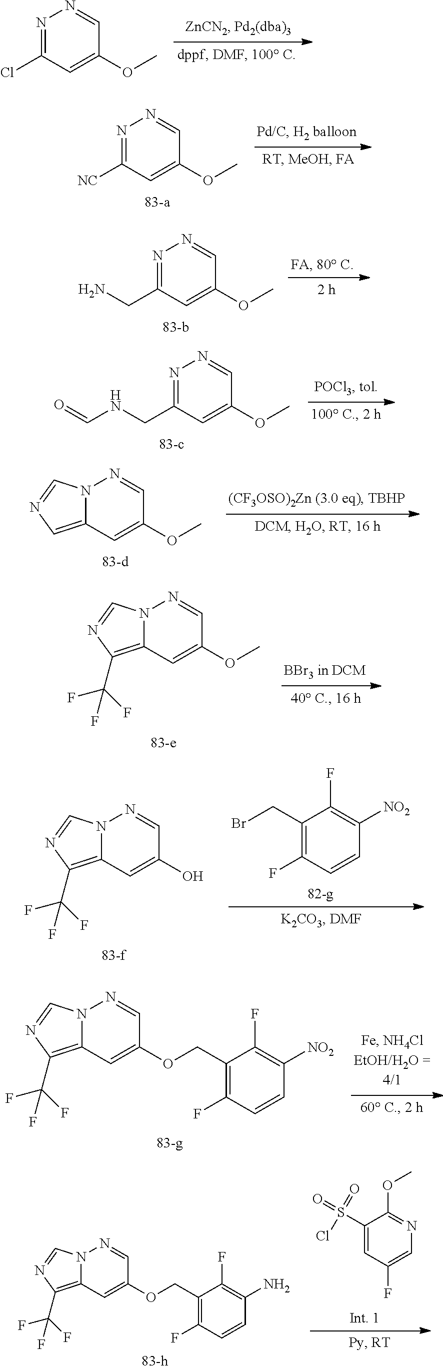

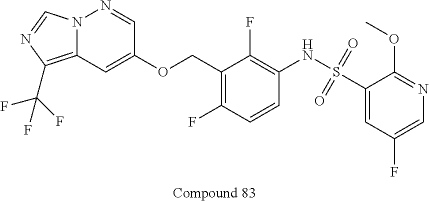 [SMILES: COc1ncc(F)cc1S(=O)(=O)Nc1ccc(F)c(COc2cnn3cnc(C(F)(F)F)c3c2)c1F]

### Synthesis of 83-a: 5-methoxypyridazine-3-carbonitrile

A mixture of 3-chloro-5-methoxypyridazine (2 g, 14 mmol, 1 equiv), Zn(CN)₂ (3.3 g, 28 mmol, 2 equiv), Pd₂(dba)₃ (1.2 g, 1.4 mmol, 0.1 equiv) and dppf (1.5 g, 2.8 mmol, 0.2 equiv) in DMF (20 mL) was stirred overnight at 100° C. under nitrogen atmosphere. The mixture was cooled and diluted with water (50 mL). This was extracted with EtOAc (3×200 mL), and the combined organics were washed with water and brine, dried over anhydrous Na₂SO₄ and concentrated. The residue was purified by silica gel column chromatography, eluting with 0-50% EtOAc/PE to afford 5-methoxypyridazine-3-carbonitrile (1.1 g, 59% yield) as a yellow solid.

LCMS (ES, m/z): [M+H]⁺: 136.

### Synthesis of 83-b: 1-(5-methoxypyridazin-3-yl)methanamine

To a solution of 5-methoxypyridazine-3-carbonitrile (1.1 g, 8.1 mmol, 1 equiv) in MeOH (11 mL) and 4 M HCl in MeOH (0.3 mL, 1.2 mmol, 0.15 equiv) was added 10% Pd/C (110 mg) under nitrogen atmosphere. The mixture was hydrogenated for 2 h under a hydrogen balloon, then was filtered through a Celite pad. The filtrate was concentrated to give crude 1-(5-methoxypyridazin-3-yl)methanamine (1.1 g).

LCMS (ES, m/z): [M+H]⁺: 140.

### Synthesis of 83-c: N-[(5-methoxypyridazin-3-yl)methyl]formamide

A solution of 1-(5-methoxypyridazin-3-yl)methanamine (1.1 g, 7.9 mmol, 1 equiv) in HCOOH (11 mL) was stirred overnight at 80° C. The resulting mixture was concentrated. The 1.3 g N-[(5-methoxypyridazin-3-yl)methyl]formamide crude product was used in the next step directly without further purification.

LCMS (ES, m/z): [M+H]⁺: 168.

### Synthesis of 83-d: 3-methoxyimidazo[1,5-b]pyridazine

To a stirred solution of N-[(5-methoxypyridazin-3-yl)methyl]formamide (1.3 g, 7.7 mmol, 1 equiv) in toluene (13 mL) was added phosphorus oxychloride (3.6 g, 23 mmol, 3 equiv) dropwise at 0-5° C. The reaction was stirred for 2 h at 100° C. then was cooled and concentrated. The reaction was quenched by the addition of water/ice (10 mL) and extracted with EtOAc (3×50 mL). The combined organics were washed with water and brine, dried over anhydrous Na₂SO₄ and concentrated. The residue was purified by silica gel column chromatography, eluting with 0-100% EtOAc/PE to afford 3-methoxyimidazo[1,5-b]pyridazine (300 mg, 26% yield) as a yellow solid.

LCMS (ES, m/z): [M+H]⁺:150.

### Synthesis of 83-e: 3-methoxy-5-(trifluoromethyl)imidazo[1,5-b]pyridazine

To a stirred solution of 3-methoxyimidazo[1,5-b]pyridazine (1 g, 6.7 mmol, 1 equiv) and trifluoromethanesulfinyl zinc (3.7 g, 20 mmol, 3 equiv) in DCM (40 mL) and H₂O (20 mL) was added 70% TBHP in H₂O (4.3 g, 34 mmol, 5 equiv) dropwise at 0-5° C. under nitrogen atmosphere. The reaction was stirred for 40 h at room temperature then diluted with water (20 mL). The resulting mixture was extracted with DCM (3×50 mL). The combined organics were washed with water and brine, dried over anhydrous Na₂SO₄ and concentrated. The residue was purified by silica gel column chromatography, eluting with 0-100% EtOAc/PE to afford 3-methoxy-5-(trifluoromethyl)imidazo[1,5-b]pyridazine (230 mg, 16% yield) as a yellow solid.

LCMS (ES, m/z): [M+H]⁺: 218.

### Synthesis of 83-f: 5-(trifluoromethyl)imidazo[1,5-b]pyridazin-3-ol

3-Methoxy-5-(trifluoromethyl)imidazo[1,5-b]pyridazine (150 mg, 0.69 mmol, 1 equiv) was treated with 1 M BBr₃ in DCM (4 mL, 4 mmol, 5.8 equiv) and stirred for 40 h at room temperature. The resulting mixture was concentrated. The 5-(trifluoromethyl)imidazo[1,5-b]pyridazin-3-ol (100 mg) crude product was used in the next step directly without further purification.

LCMS (ES, m/z): [M+H]⁺: 204.

### Synthesis of 83-g: 3-((2,6-difluoro-3-nitrobenzyl)oxy)-5-(trifluoromethyl)imidazo[1,5-b]pyridazine

To a stirred mixture of 5-(trifluoromethyl)imidazo[1,5-b]pyridazin-3-ol (50 mg, 0.1 mmol, 1 equiv) and 2-(bromomethyl)-1,3-difluoro-4-nitrobenzene (32 mg, 0.12 mmol, 1.2 equiv) in DMF (1 mL) was added K₂CO₃ (29 mg, 0.2 mmol, 2 equiv). The reaction was stirred for 4 h at 40° C., then cooled and diluted with EA (20 mL). The combined organics were washed with water and brine, dried over anhydrous Na₂SO₄ and concentrated. The residue was purified by silica gel column chromatography, eluting with PE/EA (3/1) to afford 3-[(2,6-difluoro-3-nitrophenyl)methoxy]-5-(trifluoromethyl)imidazo[1,5-b]pyridazine (13 mg, 33% yield) as a light yellow solid.

LCMS (ES, m/z): [M+H]⁺:375

### Synthesis of 83-h: 3-((2,6-difluoro-3-nitrobenzyl)oxy)-5-methylimidazo[1,5-b]pyridazine

To a stirred mixture of 3-[(2,6-difluoro-3-nitrophenyl)methoxy]-5-(trifluoromethyl)imidazo[1,5-b]pyridazine (26 mg, 0.07 mmol, 1 equiv) and Fe (12 mg, 0.21 mmol, 3 equiv) in EtOH (1 mL) and H₂O (0.25 mL) was added NH₄Cl (11 mg, 0.21 mmol, 3 equiv) at room temperature. The resulting mixture was stirred for 2 h at 60° C., then cooled and filtered. The filter cake was washed with EA (2×20 mL), and the combined filtrates washed with water and brine, dried over anhydrous Na₂SO₄ and concentrated. The residue was purified by silica gel column chromatography, eluting with EA to afford 2,4-difluoro-3-([[5-(trifluoromethyl)imidazo[1,5-b]pyridazin-3-yl]oxy]methyl)aniline (26 mg, 79% yield) as a light yellow solid.

LCMS (ES, m/z): [M+H]⁺:345.

### Synthesis of Compound 83: N-[2,4-difluoro-3-([[5-(trifluoromethyl)imidazo[1,5-b]pyridazin-3-yl]oxy]methyl)phenyl]-5-fluoro-2-methoxypyridine-3-sulfonamide

To a solution of 2,4-difluoro-3-([[5-(trifluoromethyl)imidazo[1,5-b]pyridazin-3-yl]oxy]methyl)aniline (20 mg, 0.058 mmol, 1 equiv) in pyridine (0.2 mL) was added 5-fluoro-2-methoxypyridine-3-sulfonyl chloride (52 mg, 0.23 mmol, 4 equiv) and the solution was stirred for 0.5 h at room temperature. The resulting mixture was diluted with EA (20 mL), and washed with aq NH₄Cl, water and brine. The organics were dried over anhydrous Na₂SO₄ and concentrated. The crude product was purified by Prep-HPLC with the following conditions: Column: XBridge Prep C₁₈ OBD Column, 5 μm, 19*150 mm; Mobile Phase: 28-49% MeCN/0.05% aqueous ammonia; to afford N-[2,4-difluoro-3-([[5-(trifluoromethyl)imidazo[1,5-b]pyridazin-3-yl]oxy]methyl)phenyl]-5-fluoro-2-methoxypyridine-3-sulfonamide (15 mg, 49% yield) as a white solid.

LCMS (ES, m/z): [M+H]⁺: 534.
¹H NMR (300 MHz, DMSO-d₆) δ 10.41 (s, 1H), 8.75 (s, 1H), 8.41 (d, J=3.0 Hz, 1H), 8.35 (d, J=2.7 Hz, 1H), 7.98 (dd, J=7.4, 3.0 Hz, 1H), 7.56 (d, J=2.7 Hz, 1H), 7.40 (td, J=9.0, 6.0 Hz, 1H), 7.16 (t, J=9.1 Hz, 1H), 5.26 (s, 2H), 3.89 (s, 3H).

### Example 95: Synthesis of N-[2,4-difluoro-3-[([3-phenyl-1H-pyrazolo[3,4-b]pyridin-5-yl]oxy)methyl]phenyl]-5-fluoro-2-methoxypyridine-3-sulfonamide (Compound 84)

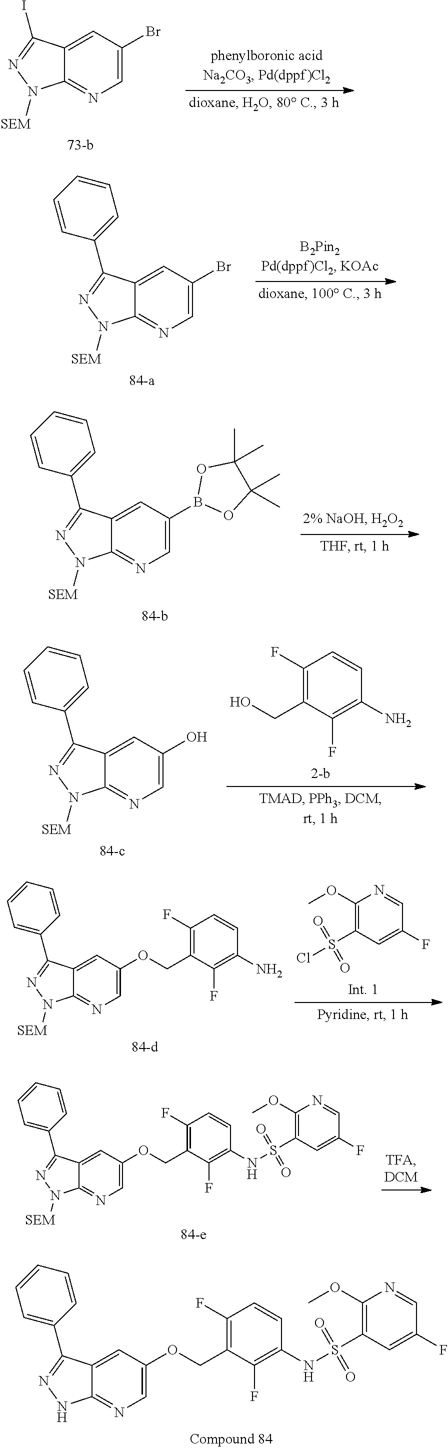

### Synthesis of 84-a: 5-bromo-3-phenyl-1-[[2(trimethylsilyl)ethoxy]methyl]pyrazolo[3,4-b]pyridine

To a solution of 5-bromo-3-iodo-1-[[2-(trimethylsilyl)ethoxy]methyl]pyrazolo[3,4-b]pyridine (800 mg, 1.8 mmol, 1 equiv) and phenyl boronic acid (215 mg, 1.8 mmol, 1 equiv) in dioxane (15 mL) and H₂O (3 mL) were added Na₂CO₃ (373 mg, 3.5 mmol, 2 equiv) and Pd(dppf)Cl₂ (129 mg, 0.17 mmol, 0.1 equiv). After stirring for 3 h at 80° C. under nitrogen atmosphere, the resulting mixture was concentrated. The residue was purified by silica gel column chromatography, eluting with PE:EA (10:1) to afford 5-bromo-3-phenyl-1-[[2 (trimethylsilyl)ethoxy]methyl]pyrazolo[3,4-b]pyridine (420 mg, 59% yield) as a yellow oil.

LCMS (ES, m/z): [M+H]⁺: 404

### Synthesis of 84 b/c: 3-phenyl-1-[[2-(trimethylsilyl) ethoxy]methyl]pyrazolo[3,4-b]pyridin-5-ol

To a solution of 5-bromo-3-phenyl-1-[[2-(trimethylsilyl)ethoxy]methyl]pyrazolo[3,4-b]pyridine (400 mg, 1 mmol, 1 equiv) and bis(pinacolato)diboron (502 mg, 2 mmol, 2 equiv) in dioxane (10 mL) were added KOAc (194 mg, 2 mmol, 2 equiv) and Pd(dppf)Cl₂ (72 mg, 0.1 mmol, 0.1 equiv). The reaction was stirred for 3 h at 100° C. under a nitrogen atmosphere, then cooled and diluted with THE (10 mL) and 2% aqueous NaOH solution (10 mL). 30% H₂O₂ (151 mg, 4.4 mmol, 5 equiv) was added dropwise, and the reaction stirred at room temperature for 1 hr. The mixture was quenched with saturated aqueous Na₂S₂O₃ solution (10 mL) and extracted with EA (3×20 mL). The combined organics were washed with brine (10 mL), dried over anhydrous Na₂SO₄ and concentrated. The residue was purified by silica gel column chromatography, eluting with PE:EA (4:1) to afford 3-phenyl-1-[[2-(trimethylsilyl) ethoxy]methyl]pyrazolo[3,4-b]pyridin-5-ol (260 mg, 86% yield) as a yellow oil.

LCMS (ES, m/z): [M+H]⁺: 342

### Synthesis of 84-d: 2,4-difluoro-3-[[(3-phenyl-1-[[2-(trimethylsilyl)ethoxy]methyl]pyrazolo[3,4-b]pyridin-5-yl)oxy]methyl]aniline

To a stirred solution of 3-phenyl-1-[[2-(trimethylsilyl)ethoxy]methyl]pyrazolo[3,4-b]pyridin-5-ol (400 mg, 1.2 mmol, 1 equiv) and (3-amino-2,6-difluorophenyl)methanol (186 mg, 1.2 mmol, 1 equiv) in DCM (20 mL) were added TMAD (303 mg, 1.8 mmol, 1.5 equiv) and PPh₃ (461 mg, 1.7 mmol, 1.5 equiv). The reaction was stirred for 1 h then concentrated. The residue was purified by Flash-Prep-HPLC with the following conditions: Column, WelFlash TM C18-I, Spherical C18 20-40 μm, 120 g; mobile phase: 30-80% MeCN/0.1% aqueous formic acid; to afford 2,4-difluoro-3-[[(3-phenyl-1-[[2-(trimethylsilyl)ethoxy]methyl]pyrazolo[3,4-b]pyridin-5-yl)oxy]methyl]aniline (380 mg, 67% yield) as a brown solid.

LCMS (ES, m/z): [M+H]⁺: 483

### Synthesis of 84-e: N-(2,4-difluoro-3-[[(3-phenyl-1-[[2-(trimethylsilyl)ethoxy]methyl]pyrazolo[3,4-b]pyridin-5-yl)oxy]methyl]phenyl)-5-fluoro-2-methoxypyridine-3-sulfonamide

To a stirred solution of 2,4-difluoro-3-[[(3-phenyl-1-[[2-(trimethylsilyl)ethoxy]methyl]pyrazolo[3,4-b]pyridin-5-yl)oxy]methyl]aniline (200 mg, 0.4 mmol, 1 equiv) in pyridine (4 mL) was added 5-fluoro-2-methoxypyridine-3-sulfonyl chloride (140 mg, 0.6 mmol, 1.5 equiv) in portions at room temperature. The resulting mixture was stirred for 1 h then concentrated. The residue was purified by Flash-Prep-HPLC with the following conditions: Column, WelFlash TM C18-I, Spherical C18 20-40 μm, 120 g; mobile phase: 30-70% MeCN/0.1% aqueous formic acid; to afford N-(2,4-difluoro-3-[[(3-phenyl-1-[[2-(trimethylsilyl)ethoxy]methyl]pyrazolo[3,4-b]pyridin-5-yl)oxy]methyl]phenyl)-5-fluoro-2-methoxypyridine-3-sulfonamide (200 mg, 72% yield) as a brown oil.

LCMS (ES, m/z): [M+H]⁺: 672

### Synthesis of Compound 84: N-[2,4-difluoro-3-[([3-phenyl-1H-pyrazolo[3,4-b]pyridin-5-yl]oxy)methyl]phenyl]-5-fluoro-2-methoxypyridine-3-sulfonamide

A solution of N-(2,4-difluoro-3-[[(3-phenyl-1-[[2-(trimethylsilyl)ethoxy]methyl]pyrazolo[3,4-b]pyridin-5-yl)oxy]methyl]phenyl)-5-fluoro-2-methoxypyridine-3-sulfonamide (200 mg, 0.3 mmol, 1 equiv) in TFA (2 mL) and DCM (4 mL) was stirred for 1 h at room temperature, then concentrated. The residue was purified by prep-HPLC with the following conditions: Column, welch Vltimate XB—C18, 50×250 mm, 10 μm; Mobile Phase: 35-70% MeCN/0.1% aqueous formic acid; to afford N-[2,4-difluoro-3-[([3-phenyl-1H-pyrazolo[3,4-b]pyridin-5-yl]oxy)methyl]phenyl]-5-fluoro-2-methoxypyridine-3-sulfonamide (20 mg, 12% yield) as a white solid.

LCMS (ES, m/z): [M+H]⁺: 542
¹H NMR (300 MHz, DMSO-d₆) δ 13.74 (s, 1H), 10.37 (s, 1H), 8.42 (d, J=2.9 Hz, 1H), 8.27 (d, J=2.6 Hz, 1H), 8.14 (d, J=2.7 Hz, 1H), 8.09-8.00 (m, 2H), 7.96 (dd, J=7.2, 2.9 Hz, 1H), 7.53 (dd, J=8.3, 6.7 Hz, 2H), 7.48-7.32 (m, 2H), 7.17 (t, J=8.9 Hz, 1H), 5.25 (s, 2H), 3.88 (s, 3H).

### Example 96: N-[2,4-difluoro-3-[([3-methyl-4-phenyl-1H-pyrazolo[3,4-b]pyridin-5-yl]oxy)methyl]phenyl]-5-fluoro-2-methoxypyridine-3-sulfonamide (Compound 85)

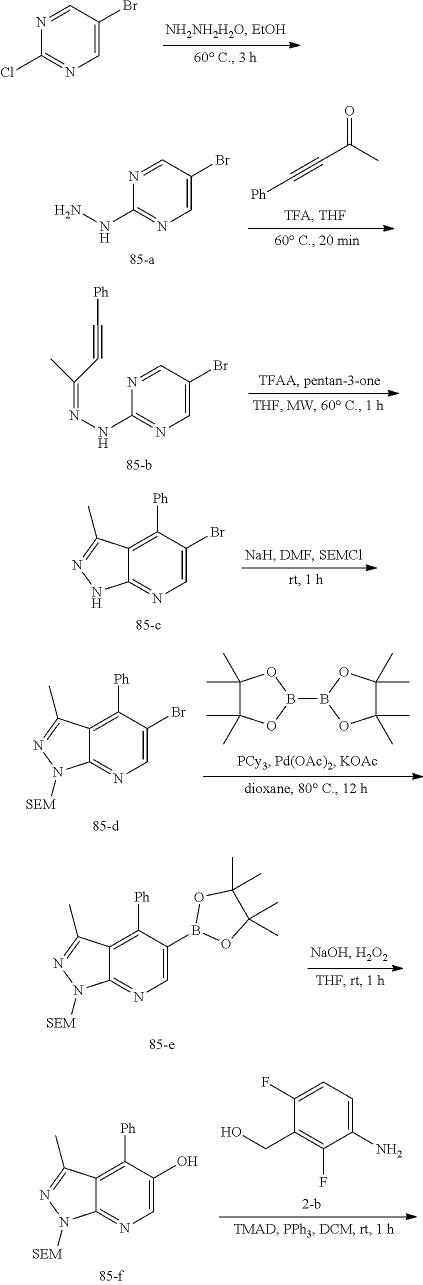

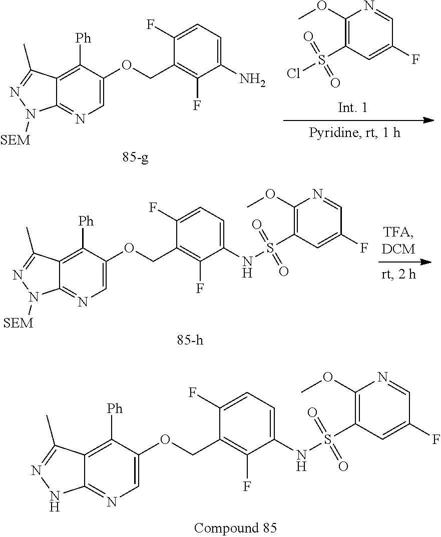 [SMILES: Cc1nn(COCC[Si](C)(C)C)c2ncc(OCc3c(F)ccc(N)c3F)c(-c3ccccc3)c12 ; COc1ncc(F)cc1S(=O)(=O)Cl ; COc1ncc(F)cc1S(=O)(=O)Nc1ccc(F)c(COc2cnc3c(c(C)nn3COCC[Si](C)(C)C)c2-c2ccccc2)c1F ; COc1ncc(F)cc1S(=O)(=O)Nc1ccc(F)c(COc2cnc3[nH]nc(C)c3c2-c2ccccc2)c1F]

### Synthesis of 85-a: 5-bromo-2-hydrazinylpyrimidine

To a solution of 5-bromo-2-chloropyrimidine (5 g, 26 mmol, 1 equiv) in EtOH (17 mL) was added NH₂NH₂·H₂O (2.6 g, 52 mmol, 2 equiv). The resulting solution was stirred for 3 h at 60° C., then cooled and diluted with H₂O (10 mL). The resulting solution was extracted with dichloromethane (8×10 mL). The combined organics were washed with brine (10 mL), dried over anhydrous Na₂SO₄ and concentrated. 5-Bromo-2-hydrazinylpyrimidine (4 g) was isolated as a white solid which was used in next step directly without further purification.

LCMS (ES, m/z): [M+H]⁺: 189

### Synthesis of 85-c: 5-bromo-3-methyl-4-phenyl-1H-pyrazolo[3,4-b]pyridine

To a solution of 5-bromo-2-hydrazinylpyrimidine (2 g, 11 mmol, 1 equiv) and 4-phenyl-3-butyn-2-one (1.5 g, 11 mmol, 1 equiv) in THE (30 mL) was added TFA (60 mg, 0.5 mmol, 0.05 equiv). The reaction was stirred for 20 min at 60° C., then concentrated. To the residue was added THE (20 mL), TFAA (3.56 g, 17 mmol, 3 equiv) and pentan-3-one (1.46 g, 17 mmol, 3 equiv). The resulting solution was heated with microwave irradiation for 1 h at 60° C., then diluted with EA (10 mL) and washed with Na₂CO₃ (2×10 mL). The organics were dried over anhydrous sodium sulfate and concentrated. The residue was purified by prep-HPLC with the following conditions: Column, WelFlash TM C18-I, Spherical C18 20-40 μm, 120 g; mobile phase: 30-80% MeCN/0.1% aqueous formic acid; to afford 5-bromo-3-methyl-4-phenyl-1H-pyrazolo[3,4-b]pyridine (1.2 g, 74% yield) as a brown solid.

LCMS (ES, m/z): [M+H]⁺: 288

### Synthesis of 85-d: 5-bromo-3-methyl-4-phenyl-1-[[2-(trimethylsilyl)ethoxy]methyl]pyrazolo[3,4-b]pyridine

To a solution of 5-bromo-3-methyl-4-phenyl-1H-pyrazolo[3,4-b]pyridine (1 g, 3.5 mmol, 1 equiv) in THE (20 mL) cooled in an ice bath was added NaH (166 mg, 4 mmol, 1.2 equiv, 60% in oil) in portions. After stirring for 30 min, the ice bath was removed and SEM-C₁ (694 mg, 4 mmol, 1.2 equiv) was added dropwise at room temperature. The resulting solution was stirred for 1 h then diluted with H₂O (10 mL). The mixture was extracted with ethyl acetate (3×10 mL), and the combined organics were washed with brine (10 mL), dried over anhydrous Na₂SO₄ and concentrated. The residue was purified by silica gel column chromatography, eluting with PE:EA (9:1) to afford 5-bromo-3-methyl-4-phenyl-1-[[2-(trimethylsilyl)ethoxy]methyl]pyrazolo[3,4-b]pyridine (1.4 g, 96% yield) as a red oil.

LCMS (ES, m/z): [M+H]⁺: 418

### Synthesis of 85-f: 3-methyl-4-phenyl-1-[[2-(trimethylsilyl)ethoxy]methyl]pyrazolo[3,4-b]pyridin-5-ol

To a solution of 5-bromo-3-methyl-4-phenyl-1-[[2-(trimethylsilyl)ethoxy]methyl]pyrazolo[3,4-b]pyridine (1.4 g, 3 mmol, 1 equiv) and bis(pinacolato)diboron (1.7 g, 6 mmol, 2 equiv) in dioxane (33 mL) were added Pd(OAc)₂ (75 mg, 0.34 mmol, 0.1 equiv), PCy₃ (188 mg, 0.67 mmol, 0.2 equiv) and KOAc (657 mg, 6.7 mmol, 2 equiv). The resulting mixture was stirred for 12 h at 80° C. under nitrogen atmosphere. The mixture was allowed to cool to room temperature and THE (15 mL) and aq. NaOH (15 mL, 2% aqueous solution) were added. 30% H₂O₂ (1 g, 30 mmol, 10 equiv) was then added dropwise at room temperature. The resulting mixture was stirred for 1 h, then quenched with saturated aqueous Na₂S₂O₃ (10 mL). The resulting mixture was extracted with EA (3×20 mL). The combined organics were washed with brine (10 mL), dried over anhydrous Na₂SO₄ and concentrated. The residue was purified by prep-HPLC Flash-Prep-HPLC with the following conditions: Column, WelFlash TM C18-I, Spherical C18 20-40 μm, 120 g; mobile phase: 30-80% MeCN/0.1% aqueous formic acid; to afford 3-methyl-4-phenyl-1-[[2-(trimethylsilyl)ethoxy]methyl]pyrazolo[3,4-b]pyridin-5-ol (788 mg, 74% yield) as a yellow solid.

LCMS (ES, m/z): [M+H]⁺: 356

### Synthesis of 85-g: 2,4-difluoro-3-[[(3-methyl-4-phenyl-1-[[2-(trimethylsilyl)ethoxy]methyl]pyrazolo[3,4-b]pyridin-5-yl)oxy]methyl]aniline

To a stirred solution of 3-methyl-4-phenyl-1-[[2-(trimethylsilyl)ethoxy]methyl]pyrazolo[3,4-b]pyridin-5-ol (780 mg, 2 mmol, 1 equiv) and (3-amino-2,6-difluorophenyl)methanol (699 mg, 4 mmol, 2 equiv) in DCM (20 mL) were added TMAD (567 mg, 3 mmol, 1.5 equiv) and PPh₃ (690 mg, 2.6 mmol, 1.2 equiv) in portions at room temperature. The resulting mixture was stirred for 1 h, then concentrated. The residue was purified by Flash-Prep-HPLC with the following conditions: Column, WelFlash TM C18-I, Spherical C18 20-40 μm, 120 g; mobile phase: 30-80% MeCN/0.1% aqueous formic acid; to afford 2,4-difluoro-3-[[(3-methyl-4-phenyl-1-[[2-(trimethylsilyl)ethoxy]methyl]pyrazolo[3,4-b]pyridin-5-yl)oxy]methyl]aniline (300 mg, 28% yield) as a yellow oil.

LCMS (ES, m/z): [M+H]⁺: 497

### Synthesis of 85-h: N-(2,4-difluoro-3-[[(3-methyl-4-phenyl-1-[[2-(trimethylsilyl)ethoxy]methyl]pyrazolo[3,4-b]pyridin-5-yl)oxy]methyl]phenyl)-5-fluoro-2-methoxypyridine-3-sulfonamide

To a stirred solution of 2,4-difluoro-3-[[(3-methyl-4-phenyl-1-[[2-(trimethylsilyl)ethoxy]methyl]pyrazolo[3,4-b]pyridin-5-yl)oxy]methyl]aniline (116 mg, 0.2 mmol, 1 equiv) in pyridine (2 mL) was added 5-fluoro-2-methoxypyridine-3-sulfonyl chloride (79 mg, 0.3 mmol, 1.5 equiv) in portions at room temperature. The resulting mixture was stirred for 1 h, then concentrated. The residue was purified by Flash-Prep-HPLC with the following conditions: Column, WelFlash TM C18-I, Spherical C18 20-40 μm, 120 g; mobile phase: 30-80% MeCN/0.1% aqueous formic acid; to afford N-(2,4-difluoro-3-[[(3-methyl-4-phenyl-1-[[2-(trimethylsilyl)ethoxy]methyl]pyrazolo[3,4-b]pyridin-5-yl)oxy]methyl]phenyl)-5-fluoro-2-methoxypyridine-3-sulfonamide (120 mg, 75% yield) as a yellow solid.

LCMS (ES, m/z): [M+H]⁺: 686

### Synthesis of Compound 85: N-[2,4-difluoro-3-[([3-methyl-4-phenyl-1H-pyrazolo[3,4-b]pyridin-5-yl]oxy)methyl]phenyl]-5-fluoro-2-methoxypyridine-3-sulfonamide

A solution of N-(2,4-difluoro-3-[[(3-methyl-4-phenyl-1-[[2-(trimethylsilyl)ethoxy]methyl]pyrazolo[3,4-b]pyridin-5-yl)oxy]methyl]phenyl)-5-fluoro-2-methoxypyridine-3-sulfonamide (120 mg, 0.17 mmol, 1 equiv) in TFA (1 mL) and DCM (2 mL) was stirred for 2 h at room temperature. The resulting mixture was concentrated and the residue purified by prep-HPLC with the following conditions: Column, welch Vltimate XB—C18, 50×250 mm, 10 μm; Mobile Phase: 5-40% MeCN/0.1% aqueous formic acid; to afford N-[2,4-difluoro-3-[([3-methyl-4-phenyl-1H-pyrazolo[3,4-b]pyridin-5-yl]oxy)methyl]phenyl]-5-fluoro-2-methoxypyridine-3-sulfonamide (30 mg, 31% yield) as a light yellow solid.

LCMS (ES, m/z): [M+H]⁺: 556
¹H NMR (300 MHz, DMSO-d₆) δ 13.25 (s, 1H), 10.27 (s, 1H), 8.38 (t, J=1.5 Hz, 2H), 7.93 (dd, J=7.3, 3.0 Hz, 1H), 7.45-7.28 (m, 3H), 7.32-7.15 (m, 1H), 7.21-7.12 (m, 2H), 6.97 (t, J=8.7 Hz, 1H), 4.93 (s, 2H), 3.79 (s, 3H), 1.89 (s, 3H).

### Example 97: Synthesis of N-[3-[([4-cyclopropyl-3-methyl-1H-Pyrazolo[3,4-b]pyridin-5-yl]oxy)methyl]-2,4-difluorophenyl]-5-fluoro-2-methoxypyridine-3-sulfonamide (Compound 86)

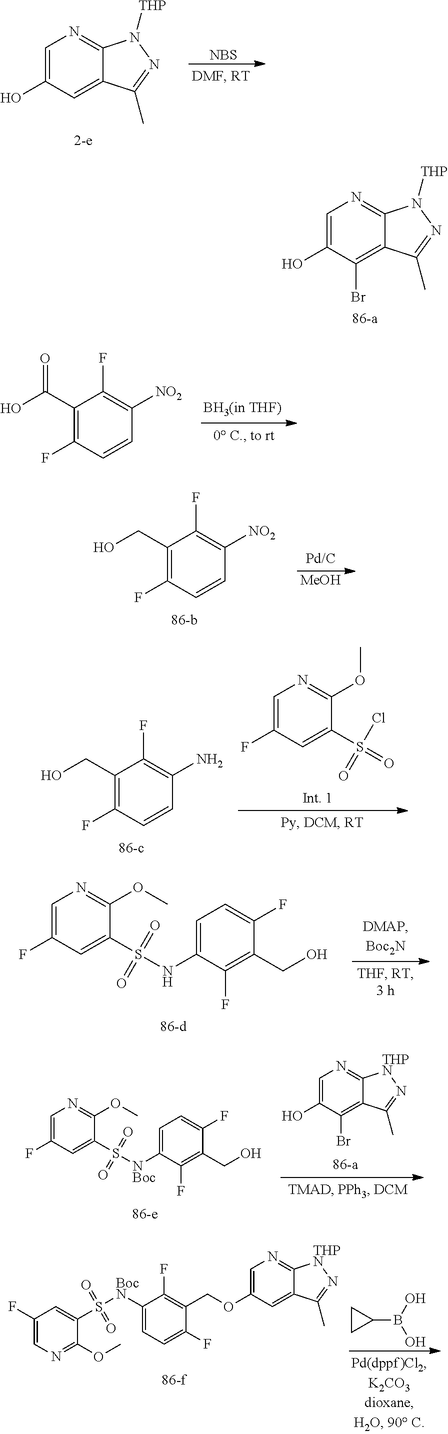

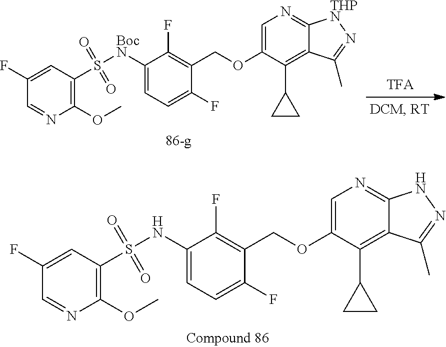

### Synthesis of 86-a: 4-bromo-3-methyl-1-(oxan-2-yl)pyrazolo[3,4-b]pyridin-5-ol

To 3-methyl-1-(oxan-2-yl)pyrazolo[3,4-b]pyridin-5-ol (18 g, 77 mmol, 1 equiv) in DMF (450 mL) was added a solution of NBS (14.8 g, 83 mmol, 1.1 equiv) in DMF (50 mL) dropwise. The resulting solution was stirred for 5 h, then diluted with ice water (1.2 L). The solids were collected by filtration, washed with water (3×200 mL) and dried under reduced pressure to give 4-bromo-3-methyl-1-(oxan-2-yl)pyrazolo[3,4-b]pyridin-5-ol (20 g, 83% yield) as a grey solid.

LCMS (ES, m/z): [M+H]⁺: 312

### Synthesis of 86-b: (2,6-difluoro-3-nitrophenyl)methanol

BH₃-THF (62 mL, 1 M, 62 mmol, 2.5 equiv) was added dropwise to a stirred solution of 2,6-difluoro-3-nitrobenzoic acid (5.0 g, 25 mmol, 1 equiv) in THE (50 mL) at 0° C. The reaction mixture was stirred 25° C. for 16 h and then quenched by the addition of MeOH (5 mL). The solution was diluted with water (100 mL) and extracted with EA (3×100 mL). The combined organic extracts were washed with brine (100 mL), dried over anhydrous sodium sulfate, filtered, and concentrated under reduced pressure. The residue was purified by silica gel column chromatography, eluting with PE:EA (5:1) to afford (2,6-difluoro-3-nitrophenyl)methanol (3.0 g, 64% yield) as a light brown solid.

LCMS (ES, m/z): [M+H]⁺: 190

### Synthesis of 86-c: (3-amino-2,6-difluorophenyl)methanol

Pd/C (0.5 g, 10% w/w) was added to a solution of (2,6-difluoro-3-nitrophenyl)methanol (1.5 g, 8 mmol, 1 equiv) in EA (30 mL) and the resulting suspension stirred at room temperature for 2 h under H₂ (2 atm). The reaction mixture was filtered and then concentrated under reduced pressure to afford (3-amino-2,6-difluorophenyl)methanol (1.0 g, 79% yield) as a brown solid.

LCMS (ES, m/z): [M+H]⁺: 160.

### Synthesis of 86-d: N-[2,4-difluoro-3-(hydroxymethyl)phenyl]-5-fluoro-2-methoxypyridine-3-sulfonamide

To (3-amino-2,6-difluorophenyl)methanol (5 g, 31 mmol, 1 equiv) in DCM (100 mL) was added 5-fluoro-2-methoxypyridine-3-sulfonyl chloride (8.5 g, 38 mmol, 1.2 equiv) and pyridine (7.5 g, 94 mmol, 3 equiv). The reaction was stirred for 1 h, then quenched with water (100 mL). The resulting solution was extracted with ethyl acetate (3×100 mL), and the combined organics washed with water (100 mL) and brine (100 mL), then dried over anhydrous sodium sulfate and concentrated. The residue was purified by silica gel chromatography, eluting with ethyl acetate/petroleum ether (1:3) to give N-[2,4-difluoro-3-(hydroxymethyl)phenyl]-5-fluoro-2-methoxypyridine-3-sulfonamide (6 g, 55% yield) as a solid.

LCMS (ES, m/z): [M+H]⁺: 349

### Synthesis of 86-e: tert-butyl N-[2,4-difluoro-3-(hydroxymethyl)phenyl]-N-(5-fluoro-2-methoxypyridin-3-ylsulfonyl)carbamate

To N-[2,4-difluoro-3-(hydroxymethyl)phenyl]-5-fluoro-2-methoxypyridine-3-sulfonamide (6.2 g, 18 mmol, 1 equiv) in THE (120 mL) was added TEA (3.6 g, 36 mmol, 2 equiv), DMAP (0.43 g, 3.5 mmol, 0.2 equiv) and BOC₂O (5.3 g, 27 mmol, 1.5 equiv). The reaction was stirred overnight, then concentrated. The residue was purified by silica gel column chromatography, eluting with ethyl acetate/petroleum ether (1:4) to give tert-butyl N-[2,4-difluoro-3-(hydroxymethyl)phenyl]-N-(5-fluoro-2-methoxypyridin-3-ylsulfonyl)carbamate (6 g, 75% yield) as a light yellow solid.

LCMS (ES, m/z): [M+H]⁺: 449

### Synthesis of 86-f: tert-butyl N-[3-([[4-bromo-3-methyl-1-(oxan-2-yl)pyrazolo[3,4-b]pyridin-5-yl]oxy]methyl)-2,4-difluorophenyl]-N-(5-fluoro-2-methoxypyridin-3-ylsulfonyl)carbamate

To tert-butyl N-[2,4-difluoro-3-(hydroxymethyl)phenyl]-N-(5-fluoro-2-methoxypyridin-3-ylsulfonyl)carbamate (4 g, 8.9 mmol, 1 equiv) in DCM (100 mL) was added 4-bromo-3-methyl-1-(oxan-2-yl)pyrazolo[3,4-b]pyridin-5-ol (2.8 g, 8.9 mmol, 1 equiv), PPh₃ (3.5 g, 13 mmol, 1.5 equiv) and TMAD (2.3 g, 13.38 mmol, 1.5 equiv). The reaction was stirred overnight, then concentrated. The residue was purified by silica gel column chromatography, eluting with ethyl acetate/petroleum ether (1:3) to give tert-butyl N-[3-([[4-bromo-3-methyl-1-(oxan-2-yl)pyrazolo[3,4-b]pyridin-5-yl]oxy]methyl)-2,4-difluorophenyl]-N-(5-fluoro-2-methoxypyridin-3-ylsulfonyl)carbamate (1.9 g, 29% yield) as a white solid.

LCMS (ES, m/z): [M+H]⁺: 742

### Synthesis of 86-g: tert-butyl N-[3-([[4-cyclopropyl-3-methyl-1-(oxan-2-yl)pyrazolo[3,4-b]pyridin-5-yl]oxy]methyl)-2,4-difluorophenyl]-N-(5-fluoro-2-methoxypyridin-3-ylsulfonyl)carbamate

To tert-butyl N-[3-([[4-bromo-3-methyl-1-(oxan-2-yl)pyrazolo[3,4-b]pyridin-5-yl]oxy]methyl)-2,4-difluorophenyl]-N-(5-fluoro-2-methoxypyridin-3-ylsulfonyl)carbamate (200 mg, 0.27 mmol, 1 equiv) in dioxane (6 mL) was added cyclopropylboronic acid (81 mg, 0.94 mmol, 3.5 equiv), Pd(dppf)Cl₂ (39 mg, 0.054 mmol, 0.2 equiv), K₂CO₃ (112 mg, 0.81 mmol, 3 equiv) and H₂O (1 mL). The reaction was stirred overnight at 90° C., then cooled and concentrated. The residue was purified by Flash-Prep-HPLC with the following conditions: Column, WelFlash TM C18-I, Spherical C18 20-40 μm, 120 g; mobile phase, 10-95% MeCN/0.1% aqueous formic acid. Concentration of the appropriate fractions gave tert-butyl N-[3-([[4-cyclopropyl-3-methyl-1-(oxan-2-yl)pyrazolo[3,4-b]pyridin-5-yl]oxy]methyl)-2,4-difluorophenyl]-N-(5-fluoro-2-methoxypyridin-3-ylsulfonyl)carbamate (120 mg, 63% yield) as a white solid.

LCMS (ES, m/z): [M+H]⁺: 704

### Synthesis of Compound 86: N-[3-[([4-cyclopropyl-3-methyl-1H-pyrazolo[3,4-b]pyridin-5-yl]oxy)methyl]-2,4-difluorophenyl]-5-fluoro-2-methoxypyridine-3-sulfonamide

To tert-butyl N-[3-([[4-cyclopropyl-3-methyl-1-(oxan-2-yl)pyrazolo[3,4-b]pyridin-5-yl]oxy]methyl)-2,4-difluorophenyl]-N-(5-fluoro-2-methoxypyridin-3-ylsulfonyl)carbamate (120 mg, 0.17 mmol, 1 equiv) in DCM (3 mL) was added TFA (1 mL). The resulting solution was stirred for 3 h, then concentrated. The pH of the solution was adjusted to 8 with NH₃ (7 mol/L in MeOH, 3 mL) before being concentrated. The residue was purified by Prep-HPLC with the following conditions: Column, welch Vltimate XB—C18, 50×250 mm, 10 μm mobile phase; Mobile Phase: 20-70% MeCN/0.05% aqueous ammonia. Concentration of the relevant fractions gave N-[3-[([4-cyclopropyl-3-methyl-1H-pyrazolo[3,4-b]pyridin-5-yl]oxy)methyl]-2,4-difluorophenyl]-5-fluoro-2-methoxypyridine-3-sulfonamide (35 mg, 40% yield) as a white solid.

LCMS (ES, m/z): [M+H]⁺: 520
¹H NMR (300 MHz, DMSO-d₆) δ 13.01 (s, 1H), 10.36 (s, 1H), 8.44 (d, 1H), 8.15 (s, 1H), 7.94 (dd, 1H), 7.36 (dt, 1H), 7.12 (dt, 1H), 5.09 (s, 2H), 3.93 (s, 3H), 2.64 (s, 3H), 2.11-1.94 (m, 1H), 1.00-0.78 (m, 4H).

### Example 98: Synthesis of N-[2,4-difluoro-3-([[3-methyl-4-(pyridin-4-yl)-1H-pyrazolo[3,4-b]pyridin-5-yl]oxy]methyl)phenyl]-5-fluoro-2-methoxypyridine-3-sulfonamide (Compound 87)

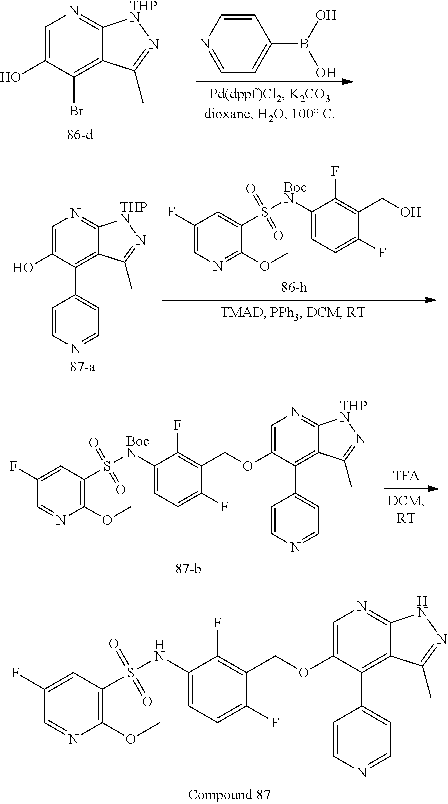

### Synthesis of 87-a: 3-methyl-1-(oxan-2-yl)-4-(pyridin-4-yl)pyrazolo[3,4-b]pyridin-5-ol

4-Bromo-3-methyl-1-(oxan-2-yl)pyrazolo[3,4-b]pyridin-5-ol (200 mg, 0.64 mmol), pyridin-4-ylboronic acid (158 mg, 1.3 mmol, 2 equiv), Pd(dppf)Cl₂ (94 mg, 0.13 mmol, 0.2 equiv) and K₂CO₃ (266 mg, 1.9 mmol, 3 equiv) in dioxane (10 mL) and H₂O (2 mL) were heated for 6 h at 100° C., then cooled and concentrated. The residue was purified by Prep-HPLC with the following conditions: Column, WelFlash TM C18-I, Spherical C18 20-40 μm, 120 g; mobile phase: 10-70% MeCN/0.1% aqueous formic acid. The appropriate fractions were concentrated to give 3-methyl-1-(oxan-2-yl)-4-(pyridin-4-yl)pyrazolo[3,4-b]pyridin-5-ol (130 mg, 6500 yield) as a white solid.

LCMS (ES, m/z): [M+H]⁺: 311

### Synthesis of 87-b: tert-butyl N-[2,4-difluoro-3-([[3-methyl-1-(oxan-2-yl)-4-(pyridin-4-yl]pyrazolo[3,4-b]pyridin-5-yl]oxy]methyl)phenyl]-N-(5-fluoro-2-methoxypyridin-3-ylsulfonyl)carbamate

3-Methyl-1-(oxan-2-yl)-4-(pyridin-4-yl)pyrazolo[3,4-b]pyridin-5-ol (150 mg, 0.48 mmol) and tert-butyl N-[2,4-difluoro-3-(hydroxymethyl)phenyl]-N-(5-fluoro-2-methoxypyridin-3-ylsulfonyl)carbamate (217 mg, 0.48 mmol, 1 equiv) in DCM (10 mL) were treated with PPh₃ (254 mg, 0.97 mmol, 2 equiv), then TMAD (167 mg, 0.97 mmol, 2 equiv). The resulting solution was stirred overnight, then concentrated. The residue was purified by Prep-HPLC with the following conditions: Column, WelFlash TM C18-I, Spherical C18 20-40 m, 120 g; mobile phase: 20-85% MeCN/0.1% aqueous formic acid. Concentration of the appropriate fractions gave tert-butyl N-[2,4-difluoro-3-([[3-methyl-1-(oxan-2-yl)-4-(pyridin-4-yl)pyrazolo[3,4-b]pyridin-5-yl]oxy]methyl)phenyl]-N-(5-fluoro-2-methoxypyridin-3-ylsulfonyl)carbamate (110 mg, 31% yield) as a white solid.

LCMS (ES, m/z): [M+H]⁺: 741

### Synthesis of Compound 87: N-[2,4-difluoro-3-([[3-methyl-4-(pyridin-4-yl)-1H-pyrazolo[3,4-b]pyridin-5-yl]oxy]methyl)phenyl]-5-fluoro-2-methoxypyridine-3-sulfonamide

Tert-butyl N-[2,4-difluoro-3-([[3-methyl-1-(oxan-2-yl)-4-(pyridin-4-yl)pyrazolo[3,4-b]pyridin-5-yl]oxy]methyl)phenyl]-N-(5-fluoro-2-methoxypyridin-3-ylsulfonyl)carbamate (140 mg, 0.19 mmol) in DCM (5 mL) was treated with TFA (1.5 mL). The resulting solution was stirred for 5 h, then concentrated. The crude product was purified by Prep-HPLC with the following conditions: Column, welch Vltimate XB—C18, 50×250 mm, 10 μm mobile phase; Mobile Phase: 20-70% MeCN/0.1% aqueous ammonia. The relevant fractions were concentrated to give N-[2,4-difluoro-3-([[3-methyl-4-(pyridin-4-yl)-1H-pyrazolo[3,4-b]pyridin-5-yl]oxy]methyl)phenyl]-5-fluoro-2-methoxypyridine-3-sulfonamide (60 mg, 57% yield) as a white solid.

LCMS (ES, m/z): [M+H]⁺: 557
¹H NMR (300 MHz, DMSO-d₆) δ 13.37 (s, 1H), 10.32 (br s, 1H), 8.62-8.50 (m, 3H), 8.36 (d, 1H), 7.92 (dd, 1H), 7.26 (td, 1H), 7.20-7.13 (m, 2H), 6.94 (td, 1H), 5.02 (s, 2H), 3.79 (s, 3H), 1.92 (s, 3H).

### Example 99: N-[2,4-difluoro-3-[([4-isopropyl-3-methyl-1H-pyrazolo[3,4-b]pyridin-5-yl]oxy)methyl]phenyl]-5-fluoro-2-methoxypyridine-3-sulfonamide (Compound 88)

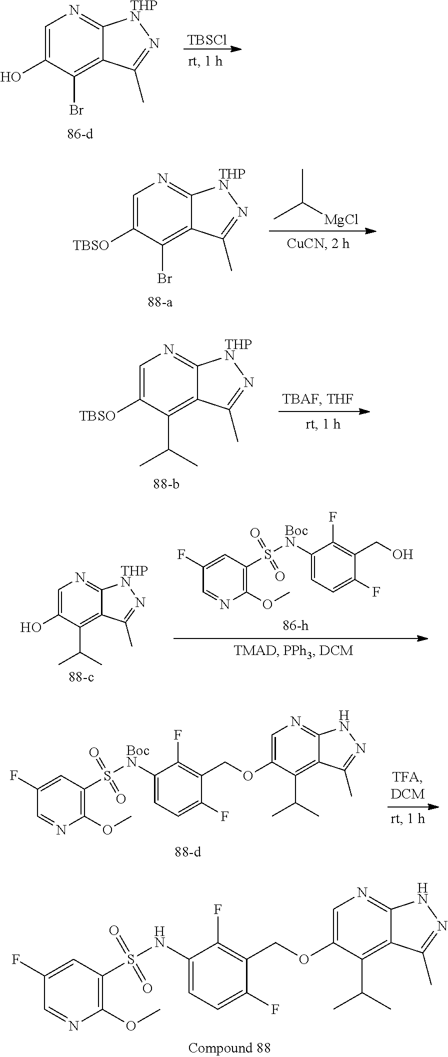

### Synthesis of 88-a: 4-bromo-5-[(tert-butyldimethylsilyl)oxy]-3-methyl-1-(oxan-2-yl)pyrazolo[3,4-b]pyridine

To a solution of 4-bromo-3-methyl-1-(oxan-2-yl)pyrazolo[3,4-b]pyridin-5-ol (8 g, 26 mmol, 1 equiv) and imidazole (5.2 g, 77 mmol, 3 equiv) in DCM (130 mL) was added TBSCl (5.79 g, 38 mmol, 1.5 equiv) at room temperature. The reaction was stirred for 1 h, then diluted with H₂O (50 mL). The resulting mixture was extracted with DCM (3×50 mL), and the combined organics were washed with brine (50 mL), dried over anhydrous Na₂SO₄, before being concentrated. The residue was purified by silica gel column chromatography, eluting with PE:EA (9:1) to afford 4-bromo-5-[(tert-butyldimethylsilyl)oxy]-3-methyl-1-(oxan-2-yl)pyrazolo[3,4-b]pyridine (10 g, 92% yield) as a white solid.

LCMS (ES, m/z): [M+H]⁺: 426

### Synthesis of 88-b: 5-[(tert-butyldimethylsilyl)oxy]-4-isopropyl-3-methyl-1-(oxan-2-yl)pyrazolo[3,4-b]pyridine

To a solution of 4-bromo-5-[(tert-butyldimethylsilyl)oxy]-3-methyl-1-(oxan-2-yl)pyrazolo[3,4-b]pyridine (2 g, 4.7 mmol, 1 equiv) and CuCN (1.3 g, 14 mmol, 3 equiv) in THE (10 mL) was added isopropyl magnesium chloride solution (2 M in THF, 14 mL, 28 mmol, 5 equiv) dropwise at 0° C. The reaction was stirred for 2 h at 0° C., then quenched with H₂O (20 mL). The resulting mixture was extracted with EA (3×20 mL), and the combined organics washed with brine (2×20 mL), dried over anhydrous Na₂SO₄, then concentrated. The residue was purified by prep-HPLC with the following conditions: Column, WelFlash TM C18-I, Spherical C18 20-40 μm, 120 g; mobile phase: 40-90% MeCN/0.1% aqueous formic acid. Concentration of the relevant fractions gave 5-[(tert-butyldimethylsilyl)oxy]-4-isopropyl-3-methyl-1-(oxan-2-yl)pyrazolo[3,4-b]pyridine (740 mg, 41% yield) as a yellow oil.

LCMS (ES, m/z): [M+H]⁺: 391

### Synthesis of 88-c: 4-isopropyl-3-methyl-1-(oxan-2-yl)pyrazolo[3,4-b]pyridin-5-ol

To a solution of 5-[(tert-butyldimethylsilyl)oxy]-4-isopropyl-3-methyl-1-(oxan-2-yl)pyrazolo[3,4-b]pyridine (700 mg, 1.8 mmol, 1 equiv) in THE (10 mL) was added TBAF (704 mg, 2.7 mmol, 1.5 equiv) in portions at room temperature. The resulting mixture was stirred for 1 h, then concentrated. The residue was purified by Flash-Prep-HPLC with the following conditions: Column, WelFlash TM C18-I, Spherical C18 20-40 μm, 120 g; mobile phase: 20-60% MeCN/0.1% aqueous formic acid. Concentration of the relevant fractions gave 4-isopropyl-3-methyl-1-(oxan-2-yl)pyrazolo[3,4-b]pyridin-5-ol (260 mg, 53% yield) as a white solid.

LCMS (ES, m/z): [M+H]⁺: 276

### Synthesis of 88-d: tert-butyl N-[2,4-difluoro-3-([[4-isopropyl-3-methyl-1-(oxan-2-yl)pyrazolo[3,4-b]pyridin-5-yl]oxy]methyl)phenyl]-N-(5-fluoro-2-methoxypyridin-3-ylsulfonyl)carbamate

To a solution of 4-isopropyl-3-methyl-1-(oxan-2-yl)pyrazolo[3,4-b]pyridin-5-ol (250 mg, 0.91 mmol, 1 equiv), tert-butyl N-[2,4-difluoro-3-(hydroxymethyl)phenyl]-N-(5-fluoro-2-methoxypyridin-3-ylsulfonyl)carbamate (448 mg, 1 mmol, 1.1 equiv) and PPh₃ (286 mg, 1.1 mmol, 1.2 equiv) in DCM (9 mL) was added TMAD (188 mg, 1.1 mmol, 1.2 equiv) at room temperature. The resulting mixture was stirred for 30 min, then concentrated. The residue was purified by silica gel column chromatography, eluting with PE:EA (4:1) to afford tert-butyl N-[2,4-difluoro-3-([[4-isopropyl-3-methyl-1-(oxan-2-yl)pyrazolo[3,4-b]pyridin-5-yl]oxy]methyl)phenyl]-N-(5-fluoro-2-methoxypyridin-3-ylsulfonyl)carbamate (600 mg, 94% yield) as a white solid.

LCMS (ES, m/z): [M+H]⁺: 622

### Synthesis of Compound 88: N-[2,4-difluoro-3-[([4-isopropyl-3-methyl-1H-pyrazolo[3,4-b]pyridin-5-yl]oxy)methyl]phenyl]-5-fluoro-2-methoxypyridine-3-sulfonamide

To a stirred solution of tert-butyl N-[2,4-difluoro-3-([[4-isopropyl-3-methyl-1-(oxan-2-yl)pyrazolo[3,4-b]pyridin-5-yl]oxy]methyl)phenyl]-N-(5-fluoro-2-methoxypyridin-3-ylsulfonyl) carbamate (300 mg, 0.425 mmol, 1 equiv) in DCM (4 mL) was added TFA (2 mL) dropwise at room temperature. The resulting mixture was stirred for 1 h, then concentrated. The residue was purified by prep-HPLC with the following conditions: Column, welch Vltimate XB—C18, 50×250 mm, 10 μm mobile phase; Mobile Phase 30-65% MeCN/0.1% aqueous ammonia. The relevant fractions were concentrated to give N-[2,4-difluoro-3-[([4-isopropyl-3-methyl-1H-pyrazolo[3,4-b]pyridin-5-yl]oxy)methyl]phenyl]-5-fluoro-2-methoxypyridine-3-sulfonamide (100 mg, 45% yield) as a white solid.

LCMS (ES, m/z): [M+H]⁺: 522
¹H NMR (300 MHz, DMSO-d₆) δ 13.02 (s, 1H), 10.39 (s, 1H), 8.41 (d, 2H), 7.94 (dd, 1H), 7.48-7.34 (m, 1H), 7.18 (t, 1H), 5.15 (s, 2H), 3.92 (s, 3H), 3.58 (p, 1H), 2.57 (s, 3H), 1.16 (d, 6H).

### Example 100: Synthesis of N-[3-[([4-benzyl-3-methyl-1H-pyrazolo[3,4-b]pyridin-5-yl]oxy)methyl]-2,4-difluorophenyl]-5-fluoro-2-methoxypyridine-3-sulfonamide (Compound 89)

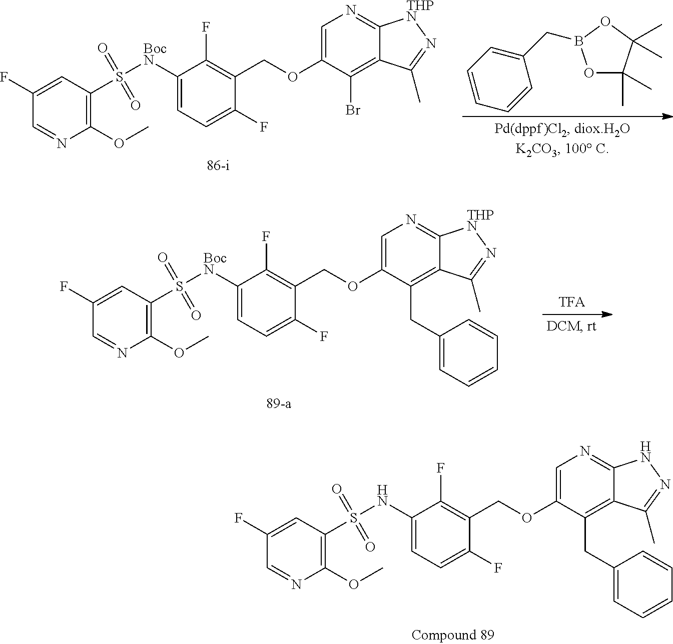

### Synthesis of 89-a: tert-butyl N-[3-([[4-benzyl-3-methyl-1-(oxan-2-yl)pyrazolo[3,4-b]pyridin-5-yl]oxy]methyl)-2,4-difluorophenyl]-N-(5-fluoro-2-methoxypyridin-3-ylsulfonyl)carbamate

Tert-butyl N-[3-([[4-bromo-3-methyl-1-(oxan-2-yl)pyrazolo[3,4-b]pyridin-5-yl]oxy]methyl)-2,4-difluorophenyl]-N-(5-fluoro-2-methoxypyridin-3-ylsulfonyl)carbamate (200 mg, 0.27 mmol), 2-benzyl-4,4,5,5-tetramethyl-1,3,2-dioxaborolane (206 mg, 0.94 mmol, 3.5 equiv), Pd(dppf)Cl₂ (59 mg, 0.08 mmol, 0.3 equiv) and K₂CO₃ (112 mg, 0.81 mmol, 3 equiv) in dioxane (6 mL) and H₂O (1 mL) were stirred for 5 h at 100° C. The mixture was concentrated in vacuo, and the residue purified by Prep-HPLC with the following conditions: Column, WelFlash TM C18-I, Spherical C18 20-40 μm, 120 g; mobile phase: 20-90% MeCN/0.1% aqueous formic acid. Concentration of the appropriate fractions gave tert-butyl N-[3-([[4-benzyl-3-methyl-1-(oxan-2-yl)pyrazolo[3,4-b]pyridin-5-yl]oxy]methyl)-2,4-difluorophenyl]-N-(5-fluoro-2-methoxypyridin-3-ylsulfonyl)carbamate (90 mg, 44% yield) as a light brown semi-solid.

LCMS (ES, m/z): [M+H]⁺: 754

### Synthesis of Compound 89: N-[3-[([4-benzyl-3-methyl-1H-pyrazolo[3,4-b]pyridin-5-yl]oxy)methyl]-2,4-difluorophenyl]-5-fluoro-2-methoxypyridine-3-sulfonamide

Tert-butyl N-[3-([[4-benzyl-3-methyl-1-(oxan-2-yl)pyrazolo[3,4-b]pyridin-5-yl]oxy]methyl)-2,4-difluorophenyl]-N-(5-fluoro-2-methoxypyridin-3-ylsulfonyl)carbamate (90 mg, 0.12 mmol) in DCM (3 mL) was treated with TFA (1 mL). The resulting solution was stirred for 3 h, then concentrated. The pH of the residue was adjusted to 8 with 7M NH₃ in MeOH, and concentrated again. The residue was purified by prep-HPLC with the following conditions: Column, welch Vltimate XB—C18, 50×250 mm, 10 μm mobile phase; Mobile Phase: 30-75% MeCN/0.1% aqueous ammonia. Concentration of the appropriate fractions gave N-[3-[([4-benzyl-3-methyl-1H-pyrazolo[3,4-b]pyridin-5-yl]oxy)methyl]-2,4-difluorophenyl]-5-fluoro-2-methoxypyridine-3-sulfonamide (15 mg 22% yield) as a white solid.

LCMS (ES, m/z): [M+H]⁺: 570
¹H NMR (300 MHz, DMSO-d₆) δ 13.12 (s, 1H), 10.35 (s, 1H), 8.45 (d, 1H), 8.38 (d, 1H), 7.93 (dd, 1H), 7.33 (dt, 1H), 7.25-7.01 (m, 4H), 6.90-6.99 (m, 2H), 5.13 (s, 2H), 4.19 (s, 2H), 3.87 (s, 3H), 2.35 (s, 3H).

### Example 101: Synthesis of N-[2,4-difluoro-3-([[3-methyl-4-(pyridin-2-yl)-1H-pyrazolo[3,4-b]pyridin-5-yl]oxy]methyl)phenyl]-5-fluoro-2-methoxypyridine-3-sulfonamide (Compound 90)

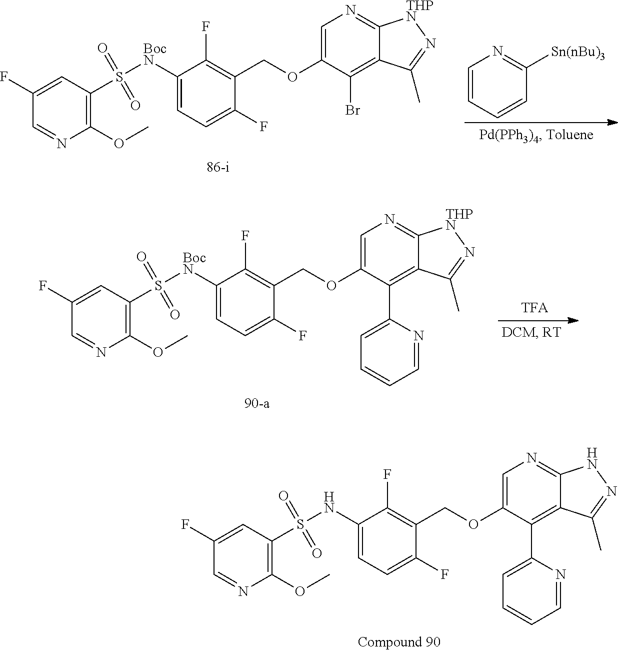

### Synthesis of 90-a: tert-butyl N-[2,4-difluoro-3-([[3-methyl-1-(oxan-2-yl)-4-(pyridin-2-yl)pyrazolo[3,4-b]pyridin-5-yl]oxy]methyl)phenyl]-N-(5-fluoro-2-methoxypyridin-3-ylsulfonyl)carbamate

Tert-butyl N-[3-([[4-bromo-3-methyl-1-(oxan-2-yl)pyrazolo[3,4-b]pyridin-5-yl]oxy]methyl)-2,4-difluorophenyl]-N-(5-fluoro-2-methoxypyridin-3-ylsulfonyl)carbamate (200 mg, 0.27 mmol) in toluene (10 mL) was treated with 2-(tributylstannyl)pyridine (198 mg, 0.54 mmol, 2 equiv) and Pd(PPh₃)₄ (63 mg, 0.054 mmol, 0.2 equiv). The resulting solution was stirred for overnight at 100° C., then concentrated. The residue was purified by Prep-HPLC with the following conditions: Column, WelFlash TM C18-I, Spherical C18 20-40 m, 120 g; mobile phase: 10-82% MeCN/0.1% aqueous formic acid. The appropriate fractions were concentrated to give tert-butyl N-[2,4-difluoro-3-([[3-methyl-1-(oxan-2-yl)-4-(pyridin-2-yl)pyrazolo[3,4-b]pyridin-5-yl]oxy]methyl)phenyl]-N-(5-fluoro-2-methoxypyridin-3-ylsulfonyl)carbamate (110 mg, 55% yield) as a light brown semi-solid.

LCMS (ES, m/z): [M+H]⁺: 741

### Synthesis of Compound 90: N-[2,4-difluoro-3-([[3-methyl-4-(pyridin-2-yl)-1H-pyrazolo[3,4-b]pyridin-5-yl]oxy]methyl)phenyl]-5-fluoro-2-methoxypyridine-3-sulfonamide

Tert-butyl N-[2,4-difluoro-3-([[3-methyl-1-(oxan-2-yl)-4-(pyridin-2-yl)pyrazolo[3,4-b]pyridin-5-yl]oxy]methyl)phenyl]-N-(5-fluoro-2-methoxypyridin-3-ylsulfonyl)carbamate (115 mg, 0.16 mmol) in DCM (3 mL) was treated with TFA (1 mL). The resulting solution was stirred for 3 h, then concentrated. The pH of the residue was adjusted to 8 with 7M NH₃ in MeOH, then concentrated again. The residue was purified by Prep-HPLC with the following conditions: Column, welch Vltimate XB—C18, 50×250 mm, 10 μm mobile phase, Mobile Phase: 20-65% MeCN/0.05% aqueous ammonia. The relevant fractions were combined and concentrated to give N-[2,4-difluoro-3-([[3-methyl-4-(pyridin-2-yl)-1H-pyrazolo[3,4-b]pyridin-5-yl]oxy]methyl)phenyl]-5-fluoro-2-methoxypyridine-3-sulfonamide (35 mg, 41% yield) as a white solid.

LCMS (ES, m/z): [M+H]⁺: 557
¹H NMR (300 MHz, DMSO-d₆) δ 13.27 (s, 1H), 10.42 (br s, 1H), 8.65 (d, 1H), 8.48 (d, 1H), 8.37 (d, 1H), 7.93 (dd, 1H), 7.82 (dt, 1H), 7.44 (dd, 1H), 7.35 (dd, 1H), 7.25 (dt, 1H), 6.95 (t, 1H), 5.00 (s, 2H), 3.80 (s, 3H), 1.88 (s, 3H).

### Example 102: Synthesis of N-[2,4-difluoro-3-([[3-methyl-4-(pyridin-3-yl)-1H-pyrazolo[3,4-b]pyridin-5-yl]oxy]methyl)phenyl]-5-fluoro-2-methoxypyridine-3-sulfonamide (Compound 91)

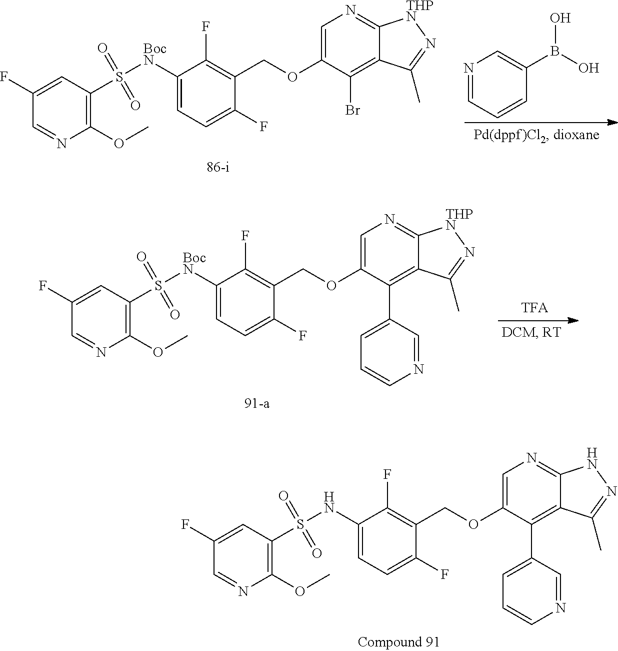

### Synthesis of 91-a: tert-butyl N-[2,4-difluoro-3-([[3-methyl-1-(oxan-2-yl)-4-(pyridin-3-yl)pyrazolo[3,4-b]pyridin-5-yl]oxy]methyl)phenyl]-N-(5-fluoro-2-methoxypyridin-3-ylsulfonyl)carbamate

Tert-butyl N-[3-([[4-bromo-3-methyl-1-(oxan-2-yl)pyrazolo[3,4-b]pyridin-5-yl]oxy]methyl)-2,4-difluorophenyl]-N-(5-fluoro-2-methoxypyridin-3-ylsulfonyl)carbamate (200 mg, 0.27 mmol), pyridin-3-ylboronic acid (66 mg, 0.54 mmol, 2 equiv), Pd(dppf)Cl₂ (60 mg, 0.08 mmol, 0.3 equiv) and K₂CO₃ (112 mg, 0.81 mmol, 3 equiv) in dioxane (6 mL) and H₂O (1 mL) were heated for 5 h at 90° C. After concentration, the residue was purified by Prep-HPLC with the following conditions: Column, WelFlash TM C18-I, Spherical C18 20-40 m, 120 g; mobile phase: 5-85% MeCN/0.1% aqueous formic acid. Concentration of the relevant fractions gave tert-butyl N-[2,4-difluoro-3-([[3-methyl-1-(oxan-2-yl)-4-(pyridin-3-yl)pyrazolo[3,4-b]pyridin-5-yl]oxy]methyl)phenyl]-N-(5-fluoro-2-methoxypyridin-3-ylsulfonyl)carbamate (130 mg, 65% yield) as a white solid.

LCMS (ES, m/z): [M+H]⁺: 741

### Synthesis of Compound 91: N-[2,4-difluoro-3-([[3-methyl-4-(pyridin-3-yl)-1H-pyrazolo[3,4-b]pyridin-5-yl]oxymethyl)phenyl]-5-fluoro-2-methoxypyridine-3-sulfonamide

Tert-butyl N-[2,4-difluoro-3-([[3-methyl-1-(oxan-2-yl)-4-(pyridin-3-yl)pyrazolo[3,4-b]pyridin-5-yl]oxy]methyl)phenyl]-N-(5-fluoro-2-methoxypyridin-3-ylsulfonyl)carbamate (140 mg, 0.19 mmol) in DCM (3 mL) was treated with TFA (1 mL). The resulting solution was stirred for 5 h, then concentrated. The pH of the residue was adjusted to 8 with 7M NH₃ in MeOH, then was concentrated again. The crude product was purified by Prep-HPLC with the following conditions: Column, welch Vltimate XB—C18, 50×250 mm, 10 μm mobile phase; Mobile Phase: 25-80% MeCN/0.05% aqueous ammonia. The relevant fractions were concentrated to give N-[2,4-difluoro-3-([[3-methyl-4-(pyridin-3-yl)-1H-pyrazolo[3,4-b]pyridin-5-yl]oxy]methyl)phenyl]-5-fluoro-2-methoxypyridine-3-sulfonamide (60 mg, 57% yield) as a white solid.

LCMS (ES, m/z): [M+H]⁺: 557
¹H NMR (300 MHz, DMSO-d₆) δ 13.34 (s, 1H), 10.35 (br s, 1H), 8.60 (d, 1H), 8.52 (s, 1H), 8.37 (dd, 2H), 7.92 (dd, 1H), 7.61 (dt, 1H), 7.41 (dd, 1H), 7.28-7.17 (m, 1H), 6.93 (t, 1H), 5.00 (s, 2H), 3.78 (s, 2H), 1.93 (s, 3H).

### Example 103: Synthesis of N-[3-[([4-cyclopentyl-3-methyl-1H-pyrazolo[3,4-b]pyridin-5-yl]oxy)methyl]-2,4-difluorophenyl]-5-fluoro-2-methoxypyridine-3-sulfonamide (Compound 92)

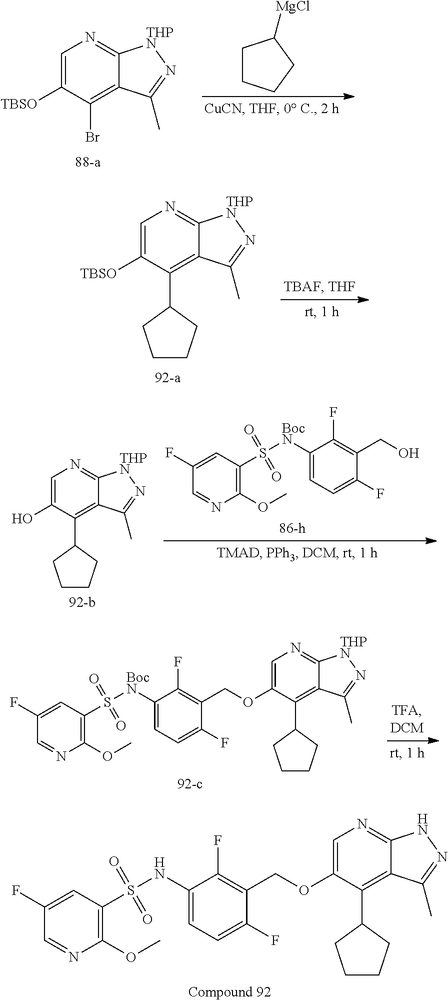

### Synthesis of 92-a: 5-[(tert-butyldimethylsilyl)oxy]-4-cyclopentyl-3-methyl-1-(oxan-2-yl)pyrazolo[3,4-b]pyridine

To a solution of 4-bromo-5-[(tert-butyldimethylsilyl)oxy]-3-methyl-1-(oxan-2-yl)pyrazolo[3,4-b]pyridine (2 g, 4.7 mmol, 1 equiv) and CuCN (1.26 g, 14 mmol, 3 equiv) in THE (20 mL) was added cyclopentyl magnesium chloride (2M in THF, 12 mL, 24 mmol, 5 equiv) dropwise at 0° C. The reaction was stirred for 2 h at 0° C., then quenched by the addition of H₂O (20 mL). The resulting mixture was extracted with EA (3×50 mL), and the combined organics washed with brine (50 mL), dried over anhydrous Na₂SO₄, before being concentrated. The residue was purified by prep-HPLC with the following conditions: Column, WelFlash TM C18-I, Spherical C18 20-40 μm, 120 g; mobile phase: 40-90% MeCN/0.1% aqueous formic acid. Concentration of the relevant fractions gave 5-[(tert-butyldimethylsilyl)oxy]-4-cyclopentyl-3-methyl-1-(oxan-2-yl)pyrazolo[3,4-b]pyridine (480 mg, 25% yield) as a yellow oil.

LCMS (ES, m/z): [M+H]⁺: 416

### Synthesis of 92-b: 4-cyclopentyl-3-methyl-1-(oxan-2-yl)pyrazolo[3,4-b]pyridin-5-ol

To a solution of 5-[(tert-butyldimethylsilyl)oxy]-4-cyclopentyl-3-methyl-1-(oxan-2-yl)pyrazolo[3,4-b]pyridine (480 mg, 1.2 mmol, 1 equiv) in THE (10 mL) was added TBAF (603 mg, 2.3 mmol, 2 equiv) in portions at room temperature. The resulting mixture was stirred for 1 h then concentrated. The residue was purified by prep-HPLC with the following conditions: Column, WelFlash TM C18-I, Spherical C18 20-40 μm, 120 g; mobile phase: 20-60% MeCN/0.1% aqueous formic acid. Concentration of the relevant fractions gave 4-cyclopentyl-3-methyl-1-(oxan-2-yl)pyrazolo[3,4-b]pyridin-5-ol (308 mg, 88% yield) as a white solid.

LCMS (ES, m/z): [M+H]⁺: 302

### Synthesis of 92-c: tert-butyl N-[3-([[4-cyclopentyl-3-methyl-1-(oxan-2-yl)pyrazolo[3,4-b]pyridin-5-yl]oxy]methyl)-2,4-difluorophenyl]-N-(5-fluoro-2-methoxypyridin-3-ylsulfonyl)carbamate

To a solution of 4-cyclopentyl-3-methyl-1-(oxan-2-yl)pyrazolo[3,4-b]pyridin-5-ol (300 mg, 1 mmol, 1 equiv), tert-butyl N-[2,4-difluoro-3-(hydroxymethyl)phenyl]-N-(5-fluoro-2-methoxypyridin-3-ylsulfonyl)carbamate (446 mg, 1 mmol, 1 equiv) and PPh₃ (313 mg, 1.2 mmol, 1.2 equiv) in DCM (10 mL) was added TMAD (205 mg, 1.2 mmol, 1.2 equiv) at room temperature. The resulting mixture was stirred for 1 h, then concentrated. The residue was purified by silica gel column chromatography, eluting with 4:1 PE:EA to afford tert-butyl N-[3-([[4-cyclopentyl-3-methyl-1-(oxan-2-yl)pyrazolo[3,4-b]pyridin-5-yl]oxy]methyl)-2,4-difluorophenyl]-N-(5-fluoro-2-methoxypyridin-3ylsulfonyl)carbamate (600 mg, 82% yield) as a white solid.

LCMS (ES, m/z): [M+H]⁺: 732

### Synthesis of Compound 92: N-[3-[([4-cyclopentyl-3-methyl-1H-pyrazolo[3,4-b]pyridin-5-yl]oxy)methyl]-2,4-difluorophenyl]-5-fluoro-2-methoxypyridine-3-sulfonamide

To a solution of tert-butyl N-[3-([[4-cyclopentyl-3-methyl-1-(oxan-2-yl)pyrazolo[3,4-b]pyridin-5-yl]oxy]methyl)-2,4-difluorophenyl]-N-(5-fluoro-2-methoxypyridin-3-ylsulfonyl)carbamate (300 mg, 0.41 mmol, 1 equiv) in DCM (4 mL) was added TFA (2 mL) dropwise at room temperature. The resulting mixture was stirred for 1 h, then concentrated. The residue was purified by prep-HPLC with the following conditions: Column, welch Vltimate XB—C18, 50×250 mm, 10 μm mobile phase; Mobile Phase: 40-60% MeCN/0.05% aqueous ammonia. Concentration of the relevant fractions gave N-[3-[([4-cyclopentyl-3-methyl-1H-pyrazolo[3,4-b]pyridin-5-yl]oxy)methyl]-2,4-difluorophenyl]-5-fluoro-2-methoxypyridine-3-sulfonamide (50 mg, 22% yield) as a white solid.

LCMS (ES, m/z): [M+H]⁺: 548
¹H NMR (300 MHz, DMSO-d₆) δ 12.98 (s, 1H), 10.38 (s, 1H), 8.44-8.37 (m, 2H), 7.93 (dd, 1H), 7.39 (dt, 1H), 7.16 (t, 1H), 5.15 (s, 2H), 3.92 (s, 3H), 3.63 (p, 1H), 2.56 (s, 3H), 1.77-1.67 (m, 4H), 1.49-1.46 (m, 4H).

### Example 104: Synthesis of N-[2,4-difluoro-3-([[3-methyl-4-(1-methylpiperidin-4-yl)-1H-pyrazolo[3,4-b]pyridin-5-yl]oxy]methyl)phenyl]-5-fluoro-2-methoxypyridine-3-sulfonamide (Compound 93)

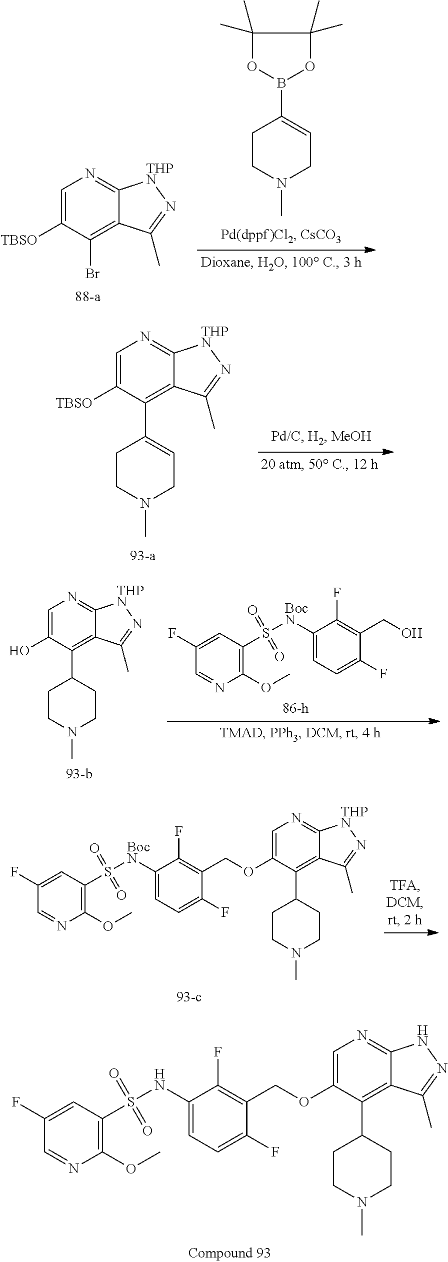

### Synthesis of 93-a: 4-[5-[(Tert-butyldimethylsilyl)oxyl-3-methyl-1-(oxan-2-yl)pyrazolo[3,4-b]pyridin-4-yl]-1-methyl-3,6-dihydro-2H-pyridine

To a solution of 4-bromo-5-[(tert-butyldimethylsilyl)oxy]-3-methyl-1-(oxan-2-yl)pyrazolo[3,4-b]pyridine (2 g, 4.7 mmol, 1 equiv), Cs₂CO₃ (4.3 g, 9.4 mmol, 2 equiv) and 1-methyl-4-(4,4,5,5-tetramethyl-1,3,2-dioxaborolan-2-yl)-3,6-dihydro-2H-pyridine (1.3 g, 5.6 mmol, 1.2 equiv) in dioxane (20 mL) and H₂O (2 mL) was added Pd(dppf)Cl₂ (0.34 g, 0.47 mmol, 0.1 equiv) in portions, at room temperature under a nitrogen atmosphere. The reaction was stirred for 3 h at 100° C., then cooled and quenched with H₂O (20 mL). The mixture was extracted with EtOAc (3×50 mL), and the combined organics washed with brine (50 mL), dried over anhydrous Na₂SO₄, and concentrated. The residue was purified by silica gel column chromatography, eluting with 3:1 PE/EtOAc to afford 4-[5-[(tert-butyldimethylsilyl)oxy]-3-methyl-1-(oxan-2-yl)pyrazolo[3,4-b]pyridin-4-yl]-1-methyl-3,6-dihydro-2H-pyridine (1.3 g, 63% yield) as a brown solid.

LCMS (ES, m/z): [M+H]⁺: 443

### Synthesis of 93-b: 3-Methyl-4-(1-methylpiperidin-4-yl)-1-(oxan-2-yl)pyrazolo[3,4-b]pyridin-5-ol

To a solution of 4-[5-[(tert-butyldimethylsilyl)oxy]-3-methyl-1-(oxan-2-yl)pyrazolo[3,4-b]pyridin-4-yl]-1-methyl-3,6-dihydro-2H-pyridine (240 mg, 0.54 mmol, 1 equiv) in MeOH (5 mL) was added and Pd/C (240 mg). The resulting mixture was stirred for 12 h at 50° C. under hydrogen (20 atm) atmosphere. After filtration, the filtrate was concentrated and the residue purified by Prep-HPLC with the following conditions: Column, WelFlash TM C18-I, Spherical C18 20-40 μm, 120 g; mobile phase: 10-50% MeCN/0.1% aqueous formic acid. Concentration of the appropriate fractions gave 3-methyl-4-(1-methylpiperidin-4-yl)-1-(oxan-2-yl)pyrazolo[3,4-b]pyridin-5-ol (100 mg, 56% yield) as a light brown solid.

LCMS (ES, m/z): [M+H]⁺: 331

### Synthesis of 93-c: Tert-butyl N-[2,4-difluoro-3-([[3-methyl-4-(1-methylpiperidin-4-yl)-1-(oxan-2-yl)pyrazolo[3,4-b]pyridin-5-yl]oxy]methyl)phenyl]-N-(5-fluoro-2-methoxypyridin-3-ylsulfonyl)carbamate

To a solution of 3-methyl-4-(1-methylpiperidin-4-yl)-1-(oxan-2-yl)pyrazolo[3,4-b]pyridin-5-ol (71 mg, 0.22 mmol), tert-butyl (2,4-difluoro-3-(hydroxymethyl)phenyl)((5-fluoro-2-methoxypyridin-3-yl)sulfonyl)carbamate (96 mg, 0.22 mmol, 1 equiv) in DCM (5 mL) were added PPh₃ (113 mg, 0.43 mmol, 2 equiv) and TMAD (74 mg, 0.43 mmol, 2 equiv) in portions at room temperature. The resulting mixture was stirred for 4 h, then quenched with H₂O (10 mL) and extracted with CH₂Cl₂ (3×20 mL). The combined organics were washed with brine (20 mL), dried over anhydrous Na₂SO₄, and concentrated. The residue was purified by Prep-HPLC with the following conditions: Column, WelFlash TM C18-I, Spherical C18 20-40 μm, 120 g; mobile phase: 20-70% MeCN/0.1% aqueous formic acid. Concentration of the appropriate fractions gave tert-butyl N-[2,4-difluoro-3-([[3-methyl-4-(1-methylpiperidin-4-yl)-1-(oxan-2-yl)pyrazolo[3,4-b]pyridin-5-yl]oxy]methyl)phenyl]-N-(5-fluoro-2-methoxypyridin-3-ylsulfonyl)carbamate (85 mg, 52% yield) as a white solid.

LCMS (ES, m/z): [M+H]⁺: 761

### Synthesis of Compound 93: N-[2,4-Difluoro-3-([[3-methyl-4-(1-methylpiperidin-4-yl)-1H-pyrazolo[3,4-b]pyridin-5-yl]oxy]methyl)phenyl]-5-fluoro-2-methoxypyridine-3-sulfonamide

A solution of tert-butyl N-[2,4-difluoro-3-([[3-methyl-4-(1-methylpiperidin-4-yl)-1-(oxan-2-yl)pyrazolo[3,4b]pyridin-5-yl]oxy]methyl)phenyl]-N-(5-fluoro-2-methoxypyridin-3-ylsulfonyl)carbamate (90 mg, 0.12 mmol) in DCM (1 mL) was treated with TFA (0.3 mL), then stirred for 2 h. The resulting mixture was concentrated and the residue purified by prep-HPLC with the following conditions: Column, welch Vltimate XB—C18, 50×250 mm, 10 μm mobile phase, Mobile Phase: 20-70% MeCN/0.1% aqueous formic acid. Concentration of the appropriate fractions gave N-[2,4-difluoro-3-([[3-methyl-4-(1-methylpiperidin-4-yl)-1H-pyrazolo[3,4-b]pyridin-5-yl]oxy]methyl)phenyl]-5-fluoro-2-methoxypyridine-3-sulfonamide (25 mg, 37% yield) as a white solid.

LCMS (ES, m/z): [M+H]⁺: 577
¹H NMR (300 MHz, DMSO-d₆) δ 13.09 (s, 1H), 8.40 (s, 1H), 8.31 (d, 1H), 7.93 (dd, 1H), 7.30 (dt, 1H), 7.03 (t, 1H), 5.16 (s, 2H), 3.84 (s, 3H), 3.27 (t, 1H), 2.92-3.04 (m, 2H), 2.62 (s, 3H), 2.32 (s, 3H), 2.31-2.03 (m, 4H), 1.48-1.62 (m, 2H).

### Example 105: Synthesis of N-[2,4-difluoro-3-([[3-methyl-4-(piperidin-1-yl)-1H-pyrazolo[3,4-b]pyridin-5-yl]oxy]methyl)phenyl]-5-fluoro-2-methoxypyridine-3-sulfonamide (Compound 94)

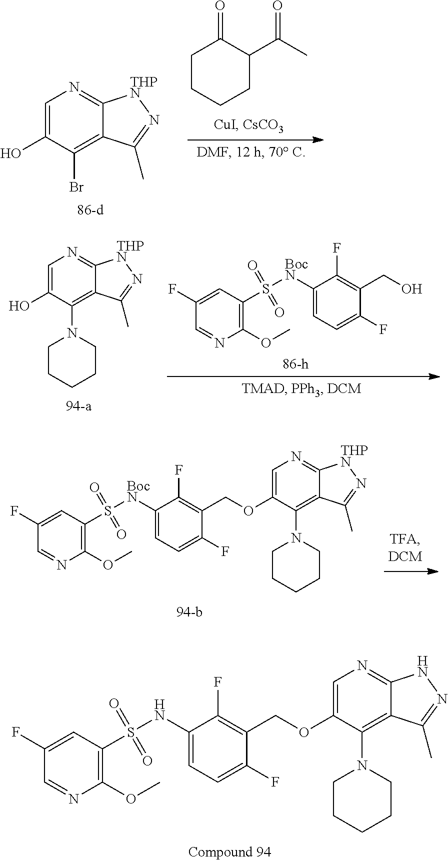

### Synthesis of 94-a: 1-[5-[(tert-butyldimethylsilyl)oxy]-3-methyl-1-(oxan-2-yl)pyrazolo[3,4-b]pyridin-4-yl]piperidine

To a solution of 4-bromo-5-[(tert-butyldimethylsilyl)oxy]-3-methyl-1-(oxan-2-yl)pyrazolo[3,4-b]pyridine (200 mg, 0.47 mmol, 1 equiv), piperidine (545 mg, 4.7 mmol, 10 equiv) and 2-acetylcyclohexan-1-one (66 mg, 0.47 mmol, 1 equiv) in DMF (4 mL) were added Cs2CO3 (458 mg, 1.4 mmol, 3 equiv) and copper (I) iodide (18 mg, 0.09 mmol, 0.2 equiv). After stirring for 12 h at 70° C. under nitrogen, the resulting mixture was concentrated. The residue was purified by Flash-Prep-HPLC with the following conditions: Column, WelFlash TM C18-I, Spherical C18 20-40 μm, 120 g; mobile phase: 30-80% MeCN/0.1% aqueous formic acid. Concentration of the relevant fractions gave 1-[5-[(tert-butyldimethylsilyl)oxy]-3-methyl-1-(oxan-2-yl)pyrazolo[3,4-b]pyridin-4-yl]piperidine (80 mg, 40% yield) as a yellow solid.

LCMS (ES, m/z): [M+H]⁺: 317

### Synthesis of 94-b: tert-butyl N-[2,4-difluoro-3-([[3-methyl-1-(oxan-2-yl)-4-(piperidin-1-yl)pyrazolo[3,4-b]pyridin-5-yl]oxy]methyl)phenyl]-N-(5-fluoro-2-methoxypyridin-3-ylsulfonyl)carbamate

To a solution of 3-methyl-1-(oxan-2-yl)-4-(piperidin-1-yl)pyrazolo[3,4-b]pyridin-5-ol (200 mg, 0.63 mmol, 1 equiv) and tert-butyl N-[2,4-difluoro-3-(hydroxymethyl)phenyl]-N-(5-fluoro-2-methoxypyridin-3-ylsulfonyl)carbamate (311 mg, 0.7 mmol, 1.1 equiv) in DCM (5 mL) were added PPh₃ (200 mg, 0.76 mmol, 1.2 equiv) and TMAD (130 mg, 0.76 mmol, 1.2 equiv) at room temperature. The resulting mixture was stirred for 2 h, then concentrated. The residue was purified by silica gel column chromatography, eluting with 1:1 PE:EA to afford tert-butyl N-[2,4-difluoro-3-([[3-methyl-1-(oxan-2-yl)-4-(piperidin-1-yl)pyrazolo[3,4-b]pyridin-5-yl]oxy]methyl)phenyl]-N-(5-fluoro-2-methoxypyridin-3-ylsulfonyl)carbamate (160 mg, 34% yield) as a yellow solid.

LCMS (ES, m/z): [M+H]⁺: 747

### Synthesis of Compound 94: N-[2,4-difluoro-3-([[3-methyl-4-(piperidin-1-yl)-1H-pyrazolo[3,4-b]pyridin-5-yl]oxy]methyl)phenyl]-5-fluoro-2-methoxypyridine-3-sulfonamide

To a solution of tert-butyl N-[2,4-difluoro-3-([[3-methyl-1-(oxan-2-yl)-4-(piperidin-1-yl)pyrazolo[3,4-b]pyridin-5-yl]oxy]methyl)phenyl]-N-(5-fluoro-2-methoxypyridin-3-ylsulfonyl)carbamate (150 mg, 0.2 mmol, 1 equiv) in DCM (5 mL) was added TFA (1 mL) dropwise. The resulting mixture was stirred for 2 h, then concentrated. The residue was purified by prep-HPLC with the following conditions: Column, welch Vltimate XB—C18, 50×250 mm, 10 um mobile phase; Mobile Phase 25-55% MeCN/0.1% aqueous formic acid. Concentration of the relevant fractions gave N-[2,4-difluoro-3-([[3-methyl-4-(piperidin-1-yl)-1H-pyrazolo[3,4-b]pyridin-5-yl]oxy]methyl)phenyl]-5-fluoro-2-methoxypyridine-3-sulfonamide (70 mg, 62% yield) as a white solid.

LCMS (ES, m/z): [M+H]⁺: 563
¹H NMR (300 MHz, DMSO-d₆) δ 12.93 (s, 1H), 10.40 (s, 1H), 8.41 (d, 1H), 8.10 (s, 1H), 7.95 (dd, 1H), 7.35 (q, 1H), 7.13 (dt, 1H), 5.00 (s, 2H), 3.89 (s, 3H), 3.0-3.12 (m, 4H), 2.55 (s, 3H), 1.45-1.66 (m, 6H).

TABLE 1

| Cmpd | | | | |
|---|---|---|---|---|
| No. | Name | Structure | Int. | LC-MS |
| 1 | 5-chloro-N-[2,4- difluoro-3-([1H- pyrazolo[3,4- b]pyridin-5- yloxy]methyl)phenyl]- 2-methoxypyridine- 3-sulfonamide | 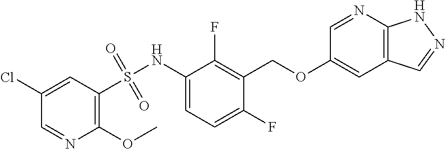 [SMILES: COc1ncc(Cl)cc1S(=O)(=O)Nc1ccc(F)c(COc2cnc3[nH]ncc3c2)c1F] | 2, 8 | [M + H]⁺: 482 |
| 2 | N-[2,4-Difluoro-3- [([3-methyl-1H- pyrazolo[3,4- b]pyridin-5- yl]oxy)methyl] phenyl]-5-fluoro-2- methoxypyridine-3- sulfonamide | 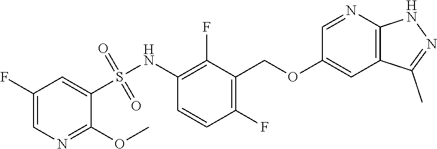 [SMILES: COc1ncc(F)cc1S(=O)(=O)Nc1ccc(F)c(COc2cnc3[nH]nc(C)c3c2)c1F] | 1 | [M + H]⁺: 480 |
| 3 | 5-chloro-N-[2,4- difluoro-3-([1H- pyrazolo[3,4- b]pyridin-5- ylamino]methyl) phenyl]-2- methoxypyridine-3- sulfonamide | 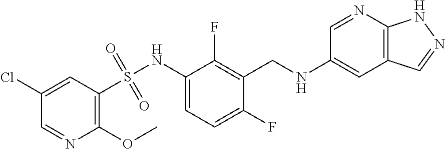 [SMILES: COc1ncc(Cl)cc1S(=O)(=O)Nc1ccc(F)c(CNc2cnc3[nH]ncc3c2)c1F] | 2 | [M + H]+: 481 |
| 4 | 5-chloro-N-(2,4- difluoro-3-[1H- pyrazolo[3,4- b]pyridin-5- ylmethoxy]phenyl)-2- methoxypyridine-3- sulfonamide | 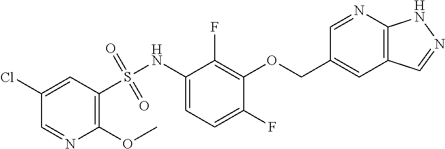 [SMILES: COc1ncc(Cl)cc1S(=O)(=O)Nc1ccc(F)c(OCc2cnc3[nH]ncc3c2)c1F] | | [M + H]⁺: 482 |
| 5 | 5-chloro-N-[2,4- difluoro-3-(2- [5H,6H,8H- imidazo[2,1- c][1,4]oxazin-3- yl]ethyl)phenyl]-2- methoxypyridine-3- sulfonamide | 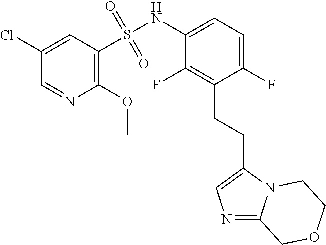 [SMILES: COc1ncc(Cl)cc1S(=O)(=O)Nc1ccc(F)c(CCc2cnc3n2CCOC3)c1F] | 2 | [M + H]⁺: 485 |
| 6 | 5-chloro-N-[2,4- difluoro-3-([1H- pyrazolo[3,4- b]pyridin-5- ylmethyl]amino) phenyl]-2- methoxypyridine-3- sulfonamide | 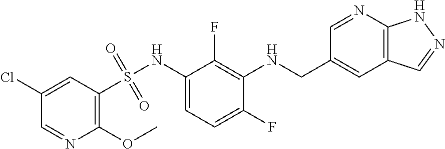 [SMILES: COc1ncc(Cl)cc1S(=O)(=O)Nc1ccc(F)c(NCc2cnc3[nH]ncc3c2)c1F] | 5 | [M + H]⁺: 481 |
| 7 | 5-chloro-N-[2,4- difluoro-3-(2-[1H- pyrazolo[3,4- b]pyridin-5- yl]ethyl)phenyl]-2- methoxypyridine-3- sulfonamide | 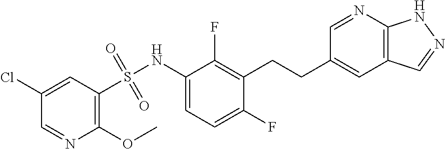 [SMILES: COc1ncc(Cl)cc1S(=O)(=O)Nc1ccc(F)c(CCc2cnc3[nH]ncc3c2)c1F] | 2, 9 | [M + H]⁺: 480 |
| 8 | 5-[2-[3-(5-Chloro-2- methoxypyridine-3- sulfonamido)-2,6- difluorophenyl]ethyl]- N-methyl-1H- pyrazole-3- carboxamide | 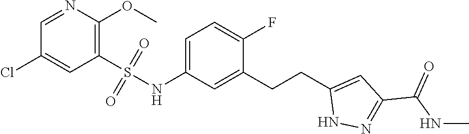 | 2, 6 | [M + H]⁺: 486 |
| 9 | N-(3-(2-(1H- pyrazolo[3,4- b]pyridin-5-yl)ethyl)- 2,4-difluorophenyl)- 5-fluoro-2- methylpyridine-3- sulfonamide | 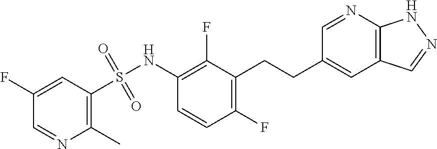 [SMILES: Cc1ncc(F)cc1S(=O)(=O)Nc1ccc(F)c(CCc2cnc3[nH]ncc3c2)c1F] | 6, 4 | [M + H]⁺: 448 |
| 10 | 5-cyano-N-[2,4- difluoro-3-(2-[1H- pyrazolo[3,4- b]pyridin-5- yl]ethyl)phenyl]-2- methoxypyridine-3- sulfonamide | 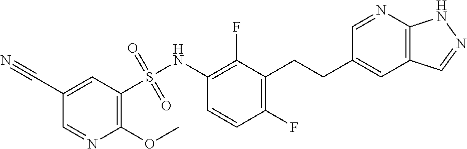 [SMILES: COc1ncc(C#N)cc1S(=O)(=O)Nc1ccc(F)c(CCc2cnc3[nH]ncc3c2)c1F] | 3, 9 | [M + H]⁺: 471 |
| 11 | 5-chloro-N-[2,4- difluoro-3-(2-[1H- pyrazolo[4,3- b]pyridin-5- yl]ethyl)phenyl]-2- methoxypyridine-3- sulfonamide | 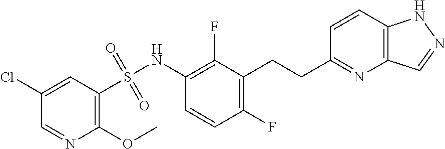 [SMILES: COc1ncc(Cl)cc1S(=O)(=O)Nc1ccc(F)c(CCc2ccc3[nH]ncc3n2)c1F] | 6, 2 | [M + H]⁺: 480 |
| 12 | 5-chloro-N-[2,4- difluoro-3-(2-[1H- pyrrolo[2,3- b]pyridin-5-yl] ethyl)phenyl]-2- methoxypyridine-3- sulfonamide | 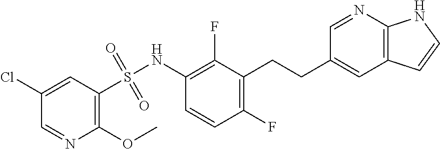 [SMILES: COc1ncc(Cl)cc1S(=O)(=O)Nc1ccc(F)c(CCc2cnc3[nH]ccc3c2)c1F] | 6, 2 | [M + H]⁺: 479 |
| 13 | 5-chloro-N-[2,4- difluoro-3-(2-[1H- pyrazolo[3,4- c]pyridin-5- yl]ethyl)phenyl]-2- methoxypyridine-3- sulfonamide | 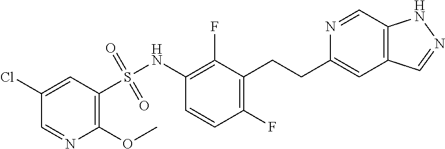 [SMILES: COc1ncc(Cl)cc1S(=O)(=O)Nc1ccc(F)c(CCc2cc3cn[nH]c3cn2)c1F] | 6, 2 | [M + H]⁺: 480 |
| 14 | 5-chloro-N-[2,4- difluoro-3-[([3- methyl-1H- pyrazolo[3,4- b]pyridin-5- yl]oxy)methyl]phenyl]- 2-methoxypyridine- 3-sulfonamide | 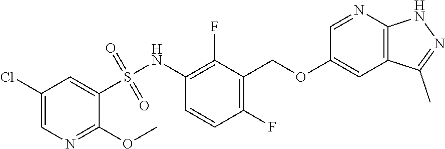 [SMILES: COc1ncc(Cl)cc1S(=O)(=O)Nc1ccc(F)c(COc2cnc3[nH]nc(C)c3c2)c1F] | 2 | [M + H]⁺: 496 |
| 15 | N-[2,4-difluoro-3-(2- [1H-pyrazolo[3,4- b]pyridin-5- yl]ethyl)phenyl]-5- fluoro-2- methoxypyridine-3- sulfonamide | 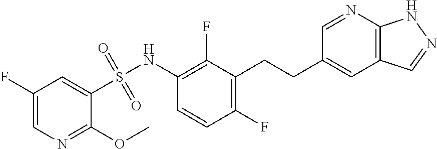 [SMILES: COc1ncc(F)cc1S(=O)(=O)Nc1ccc(F)c(CCc2cnc3[nH]ncc3c2)c1F] | 1, 9 | [M + H]⁺: 464 |
| 16 | 3-cyano-N-[2,4- difluoro-3-(2-[1H- pyrazolo[3,4- b]pyridin-5- yl]ethyl)phenyl]-5- (trifluoromethyl) benzenesulfonamide | 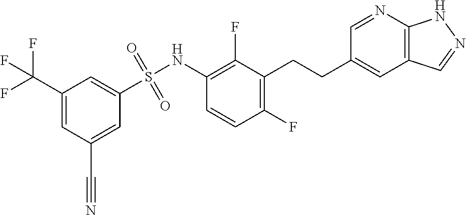 [SMILES: N#Cc1cc(C(F)(F)F)cc(S(=O)(=O)Nc2ccc(F)c(CCc3cnc4[nH]ncc4c3)c2F)c1] | 9 | [M + H]⁺: 508 |
| 17 | 5-chloro-N-[2,4- difluoro-3-(2-[2- methylpyrazolo[4,3- b]pyridin-6- yl]ethyl)phenyl]-2- methoxypyridine-3- sulfonamide | 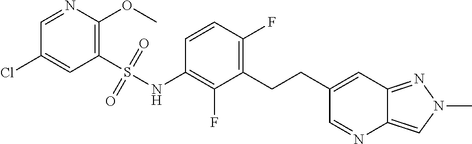 [SMILES: COc1ncc(Cl)cc1S(=O)(=O)Nc1ccc(F)c(CCc2cnc3cn(C)nc3c2)c1F] | 6, 2 | [M + H]⁺ 494 |
| 18 | 5-chloro-N-[2,4- difluoro-3-(2-[1- methylpyrazolo[4,3- b]pyridin-6- yl]ethyl)phenyl]-2- methoxypyridine-3- sulfonamide | 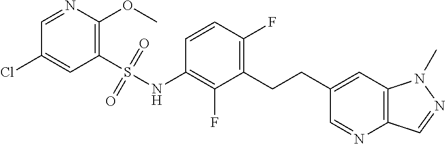 [SMILES: COc1ncc(Cl)cc1S(=O)(=O)Nc1ccc(F)c(CCc2cnc3cnn(C)c3c2)c1F] | 6, 2 | [M + H]⁺ 494 |
| 19 | 5-chloro-N-[2,4- difluoro-3-(2- [imidazo[1,5-a] pyridin-7- yl]ethyl)phenyl]-2- methoxypyridine-3- sulfonamide | 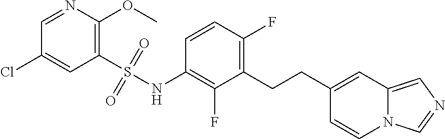 [SMILES: COc1ncc(Cl)cc1S(=O)(=O)Nc1ccc(F)c(CCc2ccn3cncc3c2)c1F] | 6, 2 | [M + H]⁺: 479 |
| 20 | N-[2,4-difluoro-3- [([4-methyl-1H- pyrazolo[3,4- b]pyridin-5- yl]oxy)methyl]phenyl]- 5-fluoro-2- methoxypyridine-3- sulfonamide | 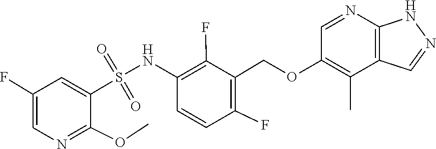 [SMILES: COc1ncc(F)cc1S(=O)(=O)Nc1ccc(F)c(COc2cnc3[nH]ncc3c2C)c1F] | 1 | [M + H]⁺: 480 |
| 21 | N-[2,4-difluoro-3- ([1H-pyrazolo[3,4- b]pyridin-5- yloxy]methyl)phenyl]- 5-fluoro-2- methoxypyridine-3- sulfonamide | 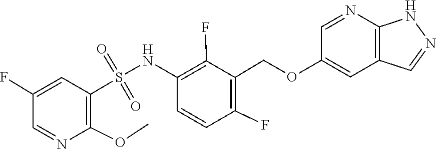 [SMILES: COc1ncc(F)cc1S(=O)(=O)Nc1ccc(F)c(COc2cnc3[nH]ncc3c2)c1F] | 1 | [M + H]⁺: 466 |
| 22 | 5-chloro-N-[2,4- difluoro-3-[([4- methyl-1H- pyrazolo[3,4- b]pyridin-5- yl]oxy)methyl]phenyl]- 2-methoxypyridine- 3-sulfonamide | 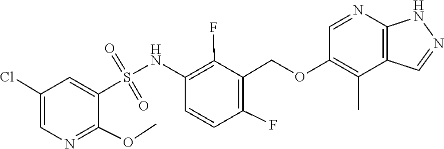 [SMILES: COc1ncc(Cl)cc1S(=O)(=O)Nc1ccc(F)c(COc2cnc3[nH]ncc3c2C)c1F] | 2 | [M + H]⁺: 496 |
| 23 | N-[2,4-difluoro-3- [([4-methoxy-1H- pyrazolo[3,4- b]pyridin-5- yl]oxy)methyl]phenyl]- 5-fluoro-2- methoxypyridine-3- sulfonamide | 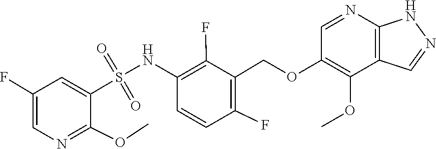 [SMILES: COc1ncc(F)cc1S(=O)(=O)Nc1ccc(F)c(COc2cnc3[nH]ncc3c2OC)c1F] | 1 | [M + H]⁺: 496 |
| 24 | 5-chloro-N-[2,4- difluoro-3-[([4- methoxy-1H- pyrazolo[3,4- b]pyridin-5- yl]oxy)methyl]phenyl]- 2-methoxypyridine- 3-sulfonamide | 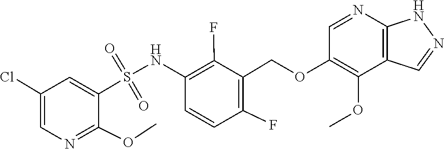 [SMILES: COc1ncc(Cl)cc1S(=O)(=O)Nc1ccc(F)c(COc2cnc3[nH]ncc3c2OC)c1F] | 2 | [M + H]⁺: 512 |
| 25 | 5-chloro-N-[2,4- difluoro-3-(1-[1H- pyrazolo[3,4- b]pyridin-5- yloxy]ethyl)phenyl]- 2-methoxypyridine-3- sulfonamide | 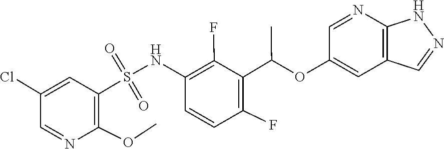 [SMILES: COc1ncc(Cl)cc1S(=O)(=O)Nc1ccc(F)c(C(C)Oc2cnc3[nH]ncc3c2)c1F] | 2 | [M + H]⁺ = 496 |
| 26 | 5-cyano-N-[2,4- difluoro-3-([1H- pyrazolo[3,4- b]pyridin-5- yloxy]methyl)phenyl]- 2-methoxypyridine- 3-sulfonamide | 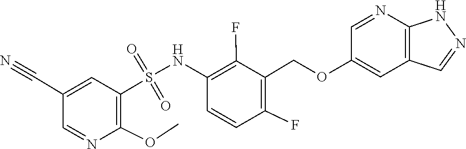 [SMILES: COc1ncc(C#N)cc1S(=O)(=O)Nc1ccc(F)c(COc2cnc3[nH]ncc3c2)c1F] | 3 | [M + H]⁺ = 473 |
| 27 | N-[3-[([4-cyano-1H- pyrazolo[3,4- b]pyridin-5- yl]oxy)methyl]-2,4- difluorophenyl]-5- fluoro-2- methoxypyridine-3- sulfonamide | 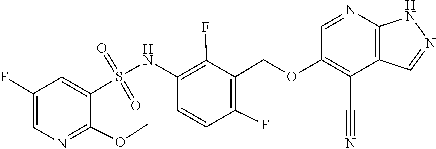 [SMILES: COc1ncc(F)cc1S(=O)(=O)Nc1ccc(F)c(COc2cnc3[nH]ncc3c2C#N)c1F] | 1 | [M + H]⁺: 491 |
| 28 | 5-chloro-N-[3-[([4- cyano-1H- pyrazolo[3,4- b]pyridin-5- yl]oxy)methyl]-2,4- difluorophenyl]-2- methoxypyridine-3- sulfonamide | 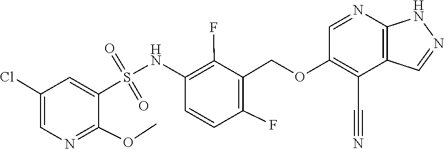 [SMILES: COc1ncc(Cl)cc1S(=O)(=O)Nc1ccc(F)c(COc2cnc3[nH]ncc3c2C#N)c1F] | 2 | [M + H]⁺: 507 |
| 29 | 5-chloro-N-[2,4- difluoro-3-(1-[1H- pyrazolo[3,4- b]pyridin-5- ylamino]ethyl)phenyl]- 2-methoxypyridine- 3-sulfonamide | 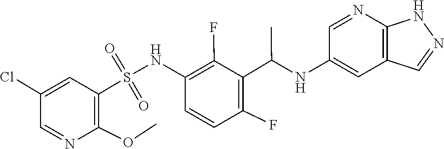 [SMILES: COc1ncc(Cl)cc1S(=O)(=O)Nc1ccc(F)c(C(C)Nc2cnc3[nH]ncc3c2)c1F] | 2 | [M + H]⁺: 495 |
| 30 | N-(3-(((1H- pyrazolo[3,4- b]pyridin-5- yl)oxy)methyl)-2,4- difluorophenyl)-5- fluoro-2- methylpyridine-3- sulfonamide | 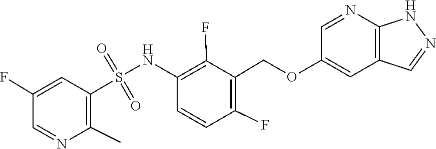 [SMILES: Cc1ncc(F)cc1S(=O)(=O)Nc1ccc(F)c(COc2cnc3[nH]ncc3c2)c1F] | 4 | [M + H]⁺: 450 |
| 31 | 5-chloro-N-(2,4- difluoro-3-(2-(8- (methylamino)imidazo [1,2-a]pyrazin-3- yl)ethyl)phenyl)-2- methoxypyridine-3- sulfonamide | 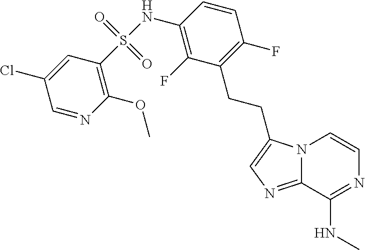 [SMILES: CNc1nccn2c(CCc3c(F)ccc(NS(=O)(=O)c4cc(Cl)cnc4OC)c3F)cnc12] | 7 | [M + H]⁺: 509 |
| 32 | N-[3-(2-[8- aminoimidazo[1,2- a]pyrazin-yl]ethyl)- 2,4-difluorophenyl]- 5-chloro-2- methoxypyridine-3- sulfonamide | 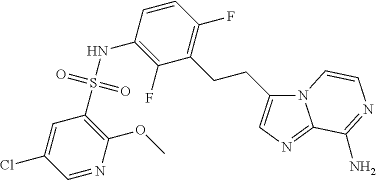 [SMILES: COc1ncc(Cl)cc1S(=O)(=O)Nc1ccc(F)c(CCc2cnc3c(N)nccn23)c1F] | 7 | [M + H]⁺: 495 |
| 33 | 5-chloro-N-[3-(2-[1- ethylpyrazolo[4,3- b]pyridin-6-yl]ethyl)- 2,4-difluorophenyl]- 2-methoxypyridine-3- sulfonamide | 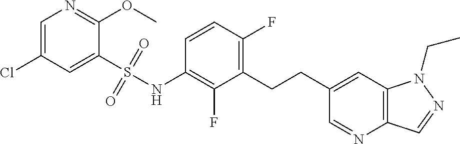 [SMILES: CCn1ncc2ncc(CCc3c(F)ccc(NS(=O)(=O)c4cc(Cl)cnc4OC)c3F)cc21] | 6, 2 | [M + H]⁺: 508 |
| 34 | N-[3-(2-[1- ethylpyrazolo[4,3- b]pyridin-6-yl]ethyl)- 2,4-difluorophenyl]- 5-fluoro-2- methoxypyridine-3- sulfonamide | 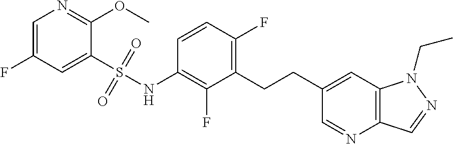 [SMILES: CCn1ncc2ncc(CCc3c(F)ccc(NS(=O)(=O)c4cc(F)cnc4OC)c3F)cc21] | 1 | [M + H]⁺: 492 |
| 35 | 5-chloro-N-[2,4- difluoro-3-(2-[1- isopropylpyrazolo[4, 3-b]pyridin-6- yl]ethyl)phenyl]-2- methoxypyridine-3- sulfonamide | 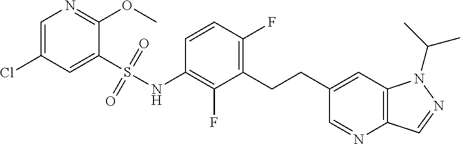 [SMILES: COc1ncc(Cl)cc1S(=O)(=O)Nc1ccc(F)c(CCc2cnc3cnn(C(C)C)c3c2)c1F] | 6, 2 | [M + H]⁺: 522 |
| 36 | N-[2,4-difluoro-3-(2- [1- isopropylpyrazolo [4,3-b]pyridin-6- yl]ethyl)phenyl]-5- fluoro-2- methoxypyridine-3- sulfonamide | 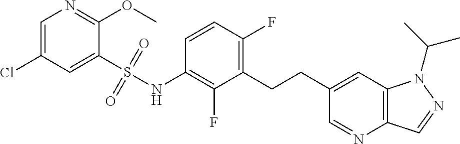 | 1 | [M + H]⁺: 506 |
| 37 | N-[2,4-difluoro-3-(2- [2- isopropylpyrazolo [4,3-b]pyridin-6- yl]ethyl)phenyl]-5- fluoro-2- methoxypyridine-3- sulfonamide | 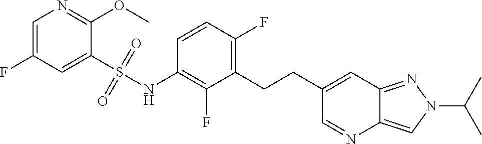 [SMILES: COc1ncc(F)cc1S(=O)(=O)Nc1ccc(F)c(CCc2cnc3cn(C(C)C)nc3c2)c1F] | 6, 1 | [M + H]⁺: 506 |
| 38 | N-[2,4-difluoro-3- [([3-methyl-1H- pyrazolo[3,4-b] pyridin-5- yl]oxy)methyl]phenyl]- 5-fluoro-2- methylpyridine-3- sulfonamide | 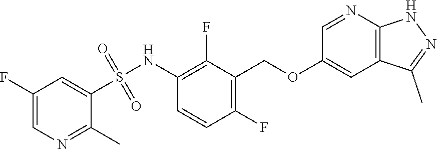 [SMILES: Cc1ncc(F)cc1S(=O)(=O)Nc1ccc(F)c(COc2cnc3[nH]nc(C)c3c2)c1F] | 4 | [M + H]⁺: 464 |
| 39 | N-[3-[([3-ethyl-1H- pyrazolo[3,4- b]pyridin-5- yl]oxy)methyl]-2,4- difluorophenyl]-5- fluoro-2- methoxypyridine-3- sulfonamide | 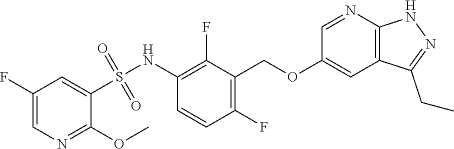 [SMILES: CCc1n[nH]c2ncc(OCc3c(F)ccc(NS(=O)(=O)c4cc(F)cnc4OC)c3F)cc12] | 1 | [M + H]⁺: 494 |
| 40 | 5-chloro-N-[3-[([3- ethyl-1H- pyrazolo[3,4- b]pyridin-5- yl]oxy)methyl]-2,4- difluorophenyl]-2- methoxypyridine-3- sulfonamide | 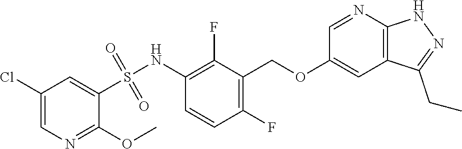 [SMILES: CCc1n[nH]c2ncc(OCc3c(F)ccc(NS(=O)(=O)c4cc(Cl)cnc4OC)c3F)cc12] | 2 | [M + H]⁺: 510 |
| 41 | 5-cyano-N-[3-[([3- ethyl-1H- pyrazolo[3,4- b]pyridin-5- yl]oxy)methyl]-2,4- difluorophenyl]-2- methoxypyridine-3- sulfonamide | 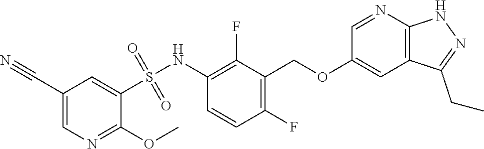 [SMILES: CCc1n[nH]c2ncc(OCc3c(F)ccc(NS(=O)(=O)c4cc(C#N)cnc4OC)c3F)cc12] | 3 | [M + H]⁺: 501 |
| 42 | 5-chloro-N-(2,4- difluoro-3-(((1- methyl-1H- pyrazolo[4,3-b] pyridin-6- yl)oxy)methyl)phenyl)- 2-methoxypyridine- 3-sulfonamide | 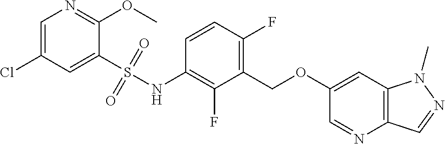 [SMILES: COc1ncc(Cl)cc1S(=O)(=O)Nc1ccc(F)c(COc2cnc3cnn(C)c3c2)c1F] | 2 | [M + H]⁺: 496 |
| 43 | N-[2,4-difluoro-3- [([1- methylpyrazolo[4,3- b]pyridin-6- yl]oxy)methyl]phenyl]- 5-fluoro-2- methoxypyridine-3- sulfonamide | 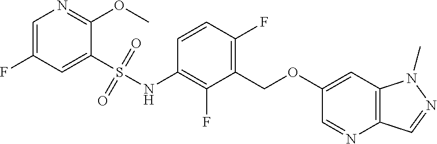 [SMILES: COc1ncc(F)cc1S(=O)(=O)Nc1ccc(F)c(COc2cnc3cnn(C)c3c2)c1F] | 1 | [M + H]⁺: 480 |
| 44 | N-[2,4-difluoro-3- ([[3- (trifluoromethyl)-1H- pyrazolo[3,4- b]pyridin-5- yl]oxy]methyl)phenyl]- 5-fluoro-2- methoxypyridine-3- sulfonamide | 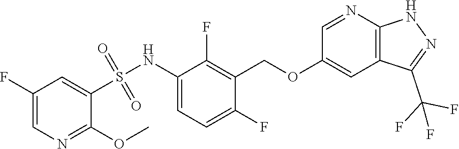 [SMILES: COc1ncc(F)cc1S(=O)(=O)Nc1ccc(F)c(COc2cnc3[nH]nc(C(F)(F)F)c3c2)c1F] | 1 | [M + H]⁺: 534 |
| 45 | N-[2,4-difluoro-3- ([3-methyl-1H- pyrazolo[3,4- b]pyridin-5- yl]methoxy)phenyl]- 5-fluoro-2- methoxypyridine-3- sulfonamide | 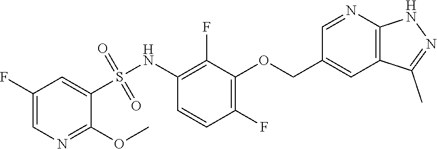 [SMILES: COc1ncc(F)cc1S(=O)(=O)Nc1ccc(F)c(OCc2cnc3[nH]nc(C)c3c2)c1F] | 1, 10 | [M + H]⁺ 480 |
| 46 | 5-chloro-N-[2,4- difluoro-3-([3- methyl-1H-pyrazolo [3,4-b]pyridin-5- yl]methoxy)phenyl]- 2-methoxypyridine-3- sulfonamide | 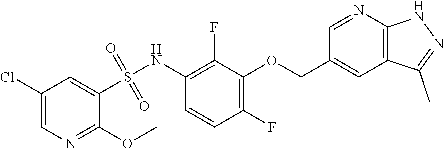 [SMILES: COc1ncc(Cl)cc1S(=O)(=O)Nc1ccc(F)c(OCc2cnc3[nH]nc(C)c3c2)c1F] | 2 | [M + H]⁺: 496 |
| 47 | N-[3-[([3-ethyl-1H- pyrazolo[3,4- b]pyridin-5- yl]oxy)methyl]-2,4- difluorophenyl]-5- fluoro-2- methylpyridine-3- sulfonamide | 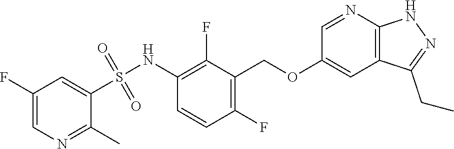 [SMILES: CCc1n[nH]c2ncc(OCc3c(F)ccc(NS(=O)(=O)c4cc(F)cnc4C)c3F)cc12] | 4 | [M + H]⁺: 478 |
| 48 | 5-cyano-N-(2,4- difluoro-3-((3- methyl-1H- pyrazolo[3,4- b]pyridin-5- yl)methoxy)phenyl)- 2-methoxypyridine-3- sulfonamide | 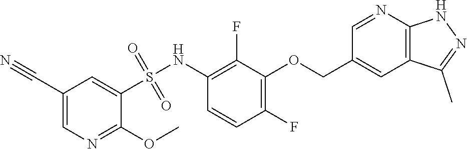 [SMILES: COc1ncc(C#N)cc1S(=O)(=O)Nc1ccc(F)c(OCc2cnc3[nH]nc(C)c3c2)c1F] | 3 | [M + H]⁺: 487 |
| 49 | 5-cyano-N-[2,4- difluoro-3-[([3- methyl-1H- pyrazolo[3,4- b]pyridin-5- yl]methyl)amino] phenyl]-2- methoxypyridine-3- sulfonamide | 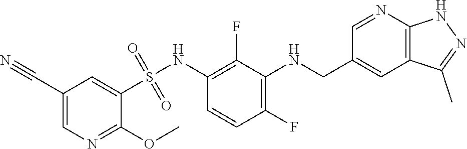 [SMILES: COc1ncc(C#N)cc1S(=O)(=O)Nc1ccc(F)c(NCc2cnc3[nH]nc(C)c3c2)c1F] | 3 | [M + H]⁺: 486 |
| 50 | 5-cyano-N-[2,4- difluoro-3-[([3- methyl-1H- pyrazolo[3,4- b]pyridin-5- yl]oxy)methyl] phenyl]-2-methoxy- pyridine-3-sulfonamide | 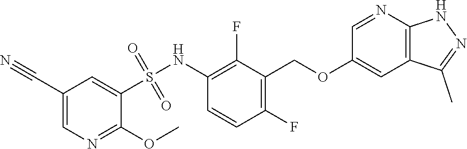 [SMILES: COc1ncc(C#N)cc1S(=O)(=O)Nc1ccc(F)c(COc2cnc3[nH]nc(C)c3c2)c1F] | 3 | [M + H]⁺: 487 |
| 51 | N-[2,4-difluoro-3- ([3-methyl-1H- pyrazolo[3,4- b]pyridin-5- yl]methoxy)phenyl]- 5-fluoro-2- methylpyridine-3- sulfonamide | 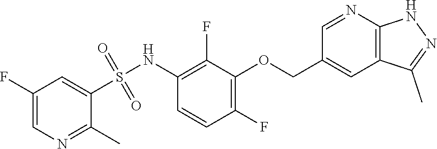 [SMILES: Cc1ncc(F)cc1S(=O)(=O)Nc1ccc(F)c(OCc2cnc3[nH]nc(C)c3c2)c1F] | 4 | [M + H]⁺: 464 |
| 52 | N-[2,4-difluoro-3- [([3-isopropyl-1H- pyrazolo[3,4- b]pyridin-5- yl]oxy)methyl]phenyl]- 5-fluoro-2- methoxypyridine-3- sulfonamide | 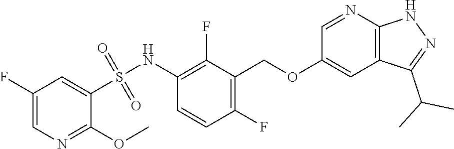 [SMILES: COc1ncc(F)cc1S(=O)(=O)Nc1ccc(F)c(COc2cnc3[nH]nc(C(C)C)c3c2)c1F] | 1 | [M + H]⁺: 508 |
| 53 | 5-chloro-N-[2,4- difluoro-3-[([3- isopropyl-1H- pyrazolo[3,4- b]pyridin-5- yl]oxy)methyl]phenyl]- 2-methoxypyridine- 3-sulfonamide | 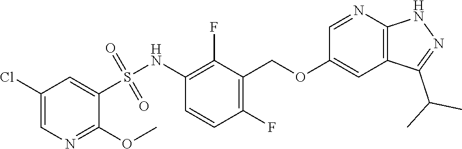 [SMILES: COc1ncc(Cl)cc1S(=O)(=O)Nc1ccc(F)c(COc2cnc3[nH]nc(C(C)C)c3c2)c1F] | 2 | [M + H]⁺: 524 |
| 54 | N-[2,4-difluoro-3- [([3-isopropyl-1H- pyrazolo[3,4- b]pyridin-5- yl]oxy)methyl]phenyl]- 5-fluoro-2- methylpyridine-3- sulfonamide | 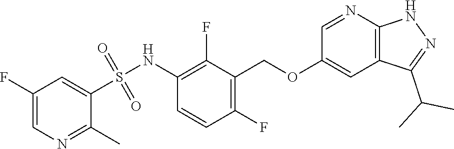 [SMILES: Cc1ncc(F)cc1S(=O)(=O)Nc1ccc(F)c(COc2cnc3[nH]nc(C(C)C)c3c2)c1F] | 4 | [M + H]⁺: 492 |
| 55 | 5-cyano-N-[2,4- difluoro-3-[([3- isopropyl-1H- pyrazolo[3,4- b]pyridin-5- yl]oxy)methyl]phenyl]- 2-methoxypyridine- 3-sulfonamide | 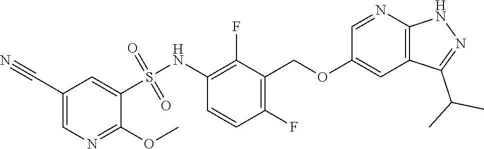 [SMILES: COc1ncc(C#N)cc1S(=O)(=O)Nc1ccc(F)c(COc2cnc3[nH]nc(C(C)C)c3c2)c1F] | 3 | [M + H]⁺: 515 |
| 56 | N-[3,5-difluoro-4- [([3-methyl-1H- pyrazolo[3,4- b]pyridin-5- yl]oxy)methyl]pyridin- 2-yl]-5-fluoro-2- methoxypyridine-3- sulfonamide | 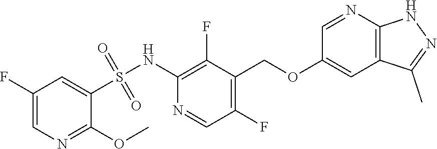 [SMILES: COc1ncc(F)cc1S(=O)(=O)Nc1ncc(F)c(COc2cnc3[nH]nc(C)c3c2)c1F] | 1, 11 | [M + H]⁺: 481 |
| 57 | N-[2,4-difluoro-3- [([3-methyl-1H- pyrazolo[3,4- b]pyridin-5- yl]methyl)amino] phenyl]-5-fluoro-2- methoxypyridine-3- sulfonamide | 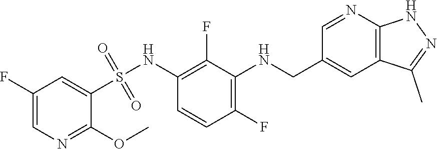 [SMILES: COc1ncc(F)cc1S(=O)(=O)Nc1ccc(F)c(NCc2cnc3[nH]nc(C)c3c2)c1F] | 1 | [M + H]⁺: 479 |
| 58 | 5-chloro-N-[2,4- difluoro-3-[([3- methyl-1H- pyrazolo[3,4- b]pyridin-5- yl]methyl)amino] phenyl]-2- methoxypyridine-3- sulfonamide | 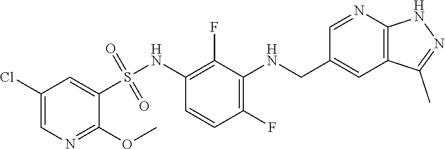 [SMILES: COc1ncc(Cl)cc1S(=O)(=O)Nc1ccc(F)c(NCc2cnc3[nH]nc(C)c3c2)c1F] | 2 | [M + H]⁺: 495 |
| 59 | N-[2,4-difluoro-3- [([3-methyl-1H- pyrazolo[3,4- b]pyridin-5- yl]methyl)amino] phenyl]-5-fluoro-2- methylpyridine-3- sulfonamide | 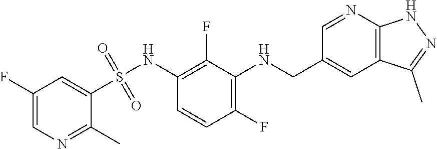 [SMILES: Cc1ncc(F)cc1S(=O)(=O)Nc1ccc(F)c(NCc2cnc3[nH]nc(C)c3c2)c1F] | 4 | [M + H]⁺: 463 |
| 60 | 5-chloro-N-[2,4- difluoro-3- ([imidazo[1,5- b]pyridazin-3- yloxy]methyl)phenyl]- 2-methoxypyridine- 3-sulfonamide | 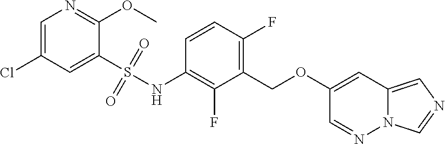 [SMILES: COc1ncc(Cl)cc1S(=O)(=O)Nc1ccc(F)c(COc2cnn3cncc3c2)c1F] | 2 | [M + H]⁺: 482 |
| 61 | N-[2-chloro-4-fluoro- 3-[([3-methyl-1H- pyrazolo[3,4- b]pyridin-5- yl]oxy)methyl]phenyl]- 5-fluoro-2- methoxypyridine-3- sulfonamide | 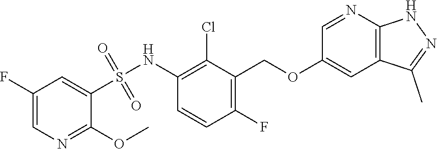 [SMILES: COc1ncc(F)cc1S(=O)(=O)Nc1ccc(F)c(COc2cnc3[nH]nc(C)c3c2)c1Cl] | 1 | [M + H]⁺: 496 |
| 62 | N-[2-cyano-4-fluoro- 3-[([3-methyl-1H- pyrazolo[3,4- b]pyridin-5- yl]oxy)methyl]phenyl]- 5-fluoro-2- methoxypyridine-3- sulfonamide | 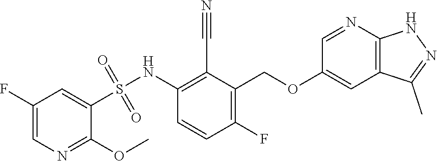 [SMILES: COc1ncc(F)cc1S(=O)(=O)Nc1ccc(F)c(COc2cnc3[nH]nc(C)c3c2)c1C#N] | | [M + H]⁺: 487 |
| 63 | 5-fluoro-N-[3-fluoro- 2-[([3-methyl-1H- pyrazolo[3,4- b]pyridin-5- yl]oxy)methyl] pyridin-4-yl]-2- methoxypyridine-3- sulfonamide | 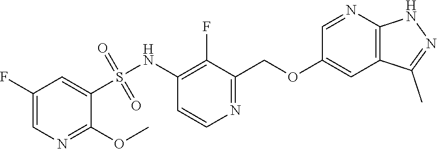 [SMILES: COc1ncc(F)cc1S(=O)(=O)Nc1ccnc(COc2cnc3[nH]nc(C)c3c2)c1F] | | [M + H]⁺: 463 |
| 64 | 5-chloro-N-[3-fluoro- 2-[([3-methyl-1H- pyrazolo[3,4- b]pyridin-5- yl]oxy)methyl] pyridin-4-yl]-2- methoxypyridine-3- sulfonamide | 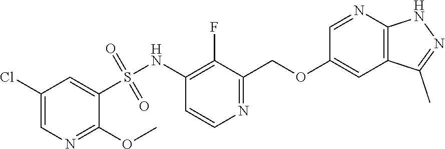 [SMILES: COc1ncc(Cl)cc1S(=O)(=O)Nc1ccnc(COc2cnc3[nH]nc(C)c3c2)c1F] | 12 | [M + H]⁺: 479 |
| 65 | 5-chloro-N-[3,5- difluoro-4-[([3- methyl-1H- pyrazolo[3,4- b]pyridin-5- yl]oxy)methyl] pyridin-2-yl]-2- methoxypyridine-3- sulfonamide | 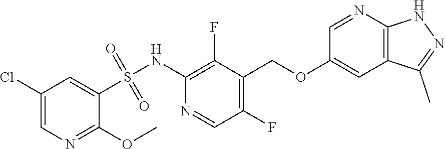 [SMILES: COc1ncc(Cl)cc1S(=O)(=O)Nc1ncc(F)c(COc2cnc3[nH]nc(C)c3c2)c1F] | 2 | [M + H]⁺: 497 |
| 66 | N-[3-chloro-5-fluoro- 4-[([3-methyl-1H- pyrazolo[3,4- b]pyridin-5- yl]oxy)methyl] pyridin-2-yl]-5-fluoro- 2- methoxypyridine-3- sulfonamide | 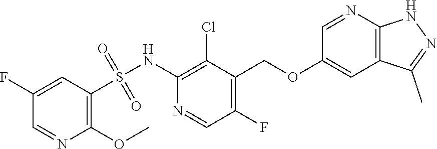 [SMILES: COc1ncc(F)cc1S(=O)(=O)Nc1ncc(F)c(COc2cnc3[nH]nc(C)c3c2)c1Cl] | | [M + H]⁺: 497 |
| 67 | 5-fluoro-N-[5-fluoro- 6-[([3-methyl-1H- pyrazolo[3,4- b]pyridin-5- yl]oxy)methyl] pyridine-2-yl]-2- methoxypyridine-3- sulfonamide | 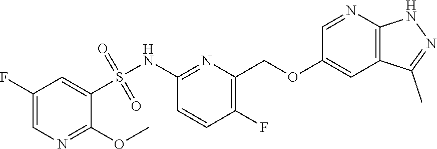 [SMILES: COc1ncc(F)cc1S(=O)(=O)Nc1ccc(F)c(COc2cnc3[nH]nc(C)c3c2)n1] | 1 | [M + H]⁺ 463 |
| 68 | 5-chloro-N-[5-fluoro- 6-[([3-methyl-1H- pyrazolo[3,4- b]pyridin-5- yl]oxy)methyl]pyridin- 2-yl]-2- methoxypyridine-3- sulfonamide | 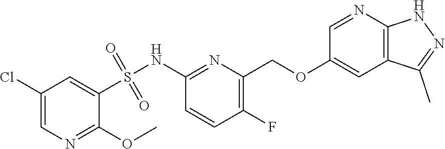 [SMILES: COc1ncc(Cl)cc1S(=O)(=O)Nc1ccc(F)c(COc2cnc3[nH]nc(C)c3c2)n1] | 2 | [M + H]⁺: 479 |
| 69 | N-[3-[([3-ethynyl- 1H-pyrazolo[3,4- b]pyridin-5- yl]oxy)methyl]-2,4- difluorophenyl]-5- fluoro-2- methoxypyridine-3- sulfonamide | 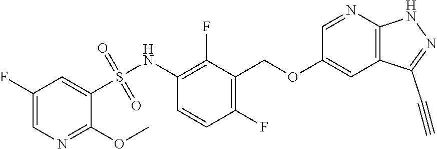 [SMILES: C#Cc1n[nH]c2ncc(OCc3c(F)ccc(NS(=O)(=O)c4cc(F)cnc4OC)c3F)cc12] | 1 | [M + H]⁺: 490 |
| 70 | N-[3-[([3- cyclopropyl-1H- pyrazolo[3,4- b]pyridin-5- yl]oxy)methyl]-2,4- difluorophenyl]-5- fluoro-2- methoxypyridine-3- sulfonamide | 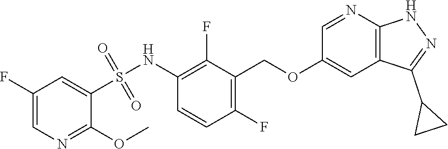 [SMILES: COc1ncc(F)cc1S(=O)(=O)Nc1ccc(F)c(COc2cnc3[nH]nc(C4CC4)c3c2)c1F] | 1 | [M + H]⁺: 506 |
| 71 | N-[2,4-difluoro-3- [([1-methyl-2-oxo- 3H-imidazo[4,5- b]pyridin-6- yl]oxy)methyl] phenyl]-5-fluoro-2- methoxypyridine-3- sulfonamide | 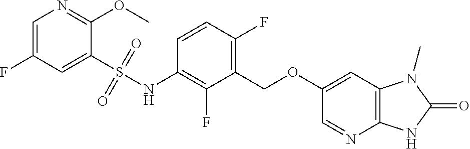 [SMILES: COc1ncc(F)cc1S(=O)(=O)Nc1ccc(F)c(COc2cnc3[nH]c(=O)n(C)c3c2)c1F] | 1 | [M + H]⁺: 496 |
| 72 | N-(2,4-difluoro-3- (((2′-oxo-1′,2′- dihydrospiro [cyclopropane-1,3′- pyrrolo[2,3- b]pyridin]-5′- yl)oxy)methyl) phenyl)-5-fluoro-2- methoxypyridine-3- sulfonamide | 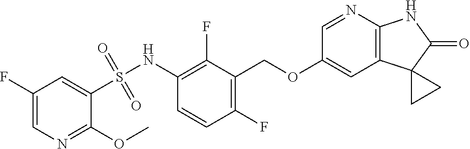 [SMILES: COc1ncc(F)cc1S(=O)(=O)Nc1ccc(F)c(COc2cnc3c(c2)C2(CC2)C(=O)N3)c1F] | 1 | [M + H]⁺: 507 |
| 73 | N-[3-[([3-cyano-1H- pyrazolo[3,4- b]pyridin-5- yl]oxy)methyl]-2,4- difluorophenyl]-5- fluoro-2- methoxypyridine-3- sulfonamide | 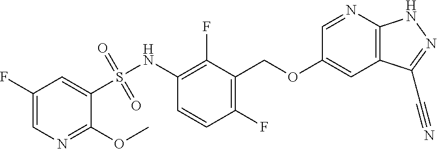 [SMILES: COc1ncc(F)cc1S(=O)(=O)Nc1ccc(F)c(COc2cnc3[nH]nc(C#N)c3c2)c1F] | 1 | [M + H]⁺: 491 |
| 74 | N-[2-chloro-4-fluoro- 3-[([3-methyl-1H- pyrazolo[3,4- b]pyridin-5- yl]oxy)methyl]phenyl]- 5-fluoro-2- methylpyridine-3- sulfonamide | 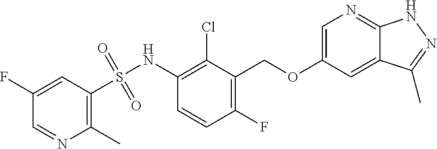 [SMILES: Cc1ncc(F)cc1S(=O)(=O)Nc1ccc(F)c(COc2cnc3[nH]nc(C)c3c2)c1Cl] | 4 | [M + H]⁺: 480 |
| 75 | N-[3-([[3-(2,2- difluoroethyl)-1H- pyrazolo[3,4- b]pyridin-5- yl]oxy]methyl)-2,4- difluorophenyl]-5- fluoro-2- methoxypyridine-3- sulfonamide | 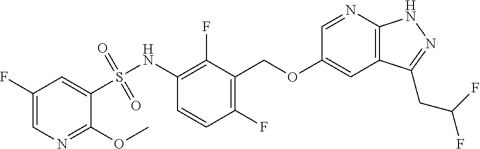 [SMILES: COc1ncc(F)cc1S(=O)(=O)Nc1ccc(F)c(COc2cnc3[nH]nc(CC(F)F)c3c2)c1F] | 1 | [M + H]⁺: 530 |
| 76 | N-[2,4-difluoro-3- ([[3-(2-fluoroethyl)- 1H-pyrazolo[3,4- b]pyridin-5- yl]oxy]methyl) phenyl)-5-fluoro-2- methoxypyridine-3- sulfonamide | 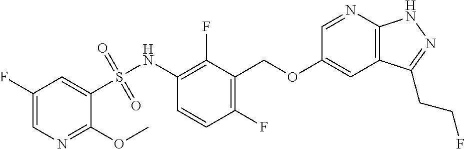 [SMILES: COc1ncc(F)cc1S(=O)(=O)Nc1ccc(F)c(COc2cnc3[nH]nc(CCF)c3c2)c1F] | 1 | [M + H]⁺: 512 |
| 77 | N-[4-chloro-2-fluoro- 3-[([3-methyl-1H- pyrazolo[3,4- b]pyridin-5- yl]oxy)methyl] phenyl]-5-fluoro-2- methylpyridine-3- sulfonamide | 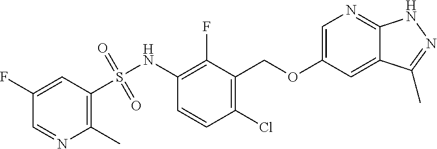 [SMILES: Cc1ncc(F)cc1S(=O)(=O)Nc1ccc(Cl)c(COc2cnc3[nH]nc(C)c3c2)c1F] | 4 | [M + H]⁺: 480 |
| 78 | N-(4-chloro-2-fluoro- 3-(((3-methyl-1H- pyrazolo[3,4- b]pyridin-5- yl)oxy)methyl) phenyl)-5-fluoro-2- methoxypyridine-3- sulfonamide | 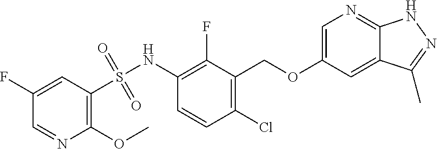 [SMILES: COc1ncc(F)cc1S(=O)(=O)Nc1ccc(Cl)c(COc2cnc3[nH]nc(C)c3c2)c1F] | 1 | [M + H]⁺: 496 |
| 79 | 5-chloro-N-[3-chloro- 5-fluoro-4-[([3- methyl-1H- pyrazolo[3,4- b]pyridin-5- yl]oxy)methyl] pyridin-2-yl]-2- methoxypyridine-3- sulfonamide | 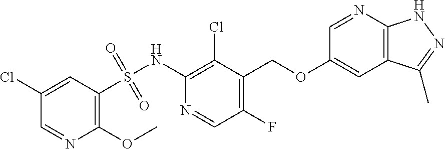 [SMILES: COc1ncc(Cl)cc1S(=O)(=O)Nc1ncc(F)c(COc2cnc3[nH]nc(C)c3c2)c1Cl] | 2 | [M + H]⁺: 513 |
| 80 | N-[2,4-difluoro-3- ([[3-(2,2,2- trifluoroethyl)-1H- pyrazolo[3,4- b]pyridin-5- yl]oxy]methyl) phenyl]-5-fluoro-2- methoxypyridine-3- sulfonamide | 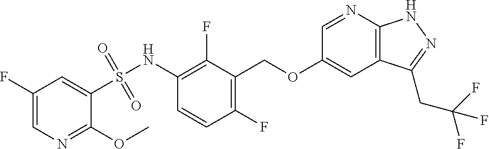 [SMILES: COc1ncc(F)cc1S(=O)(=O)Nc1ccc(F)c(COc2cnc3[nH]nc(CC(F)(F)F)c3c2)c1F] | 1 | [M + H]⁺: 548 |
| 81 | N-[3-([[3-(1,1- difluoroethyl)-1H- pyrazolo[3,4- b]pyridin-5- yl]oxy]methyl)-2,4- difluorophenyl]-5- fluoro-2- methoxypyridine-3- sulfonamide | 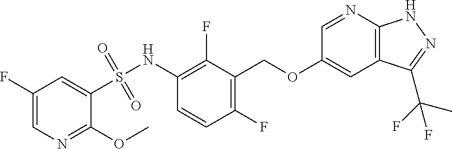 [SMILES: COc1ncc(F)cc1S(=O)(=O)Nc1ccc(F)c(COc2cnc3[nH]nc(C(C)(F)F)c3c2)c1F] | 1 | [M + H]⁺: 530 |
| 82 | N-[2,4-difluoro-3- [([5- methylimidazo[1,5- b]pyridazin-3- yl]oxy)methyl]phenyl]- 5-fluoro-2- methoxypyridine-3- sulfonamide | 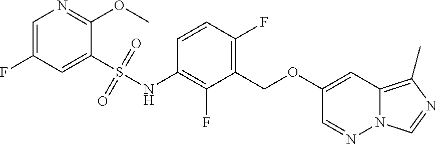 | 1 | [M + H]⁺: 515 |
| 83 | N-[2,4-difluoro-3- ([[5- (trifluoromethyl) imidazo[1,5-b] pyridazin-3-yl]oxy] methyl)phenyl]-5- fluoro-2-methoxy- pyridine-3- sulfonamide | 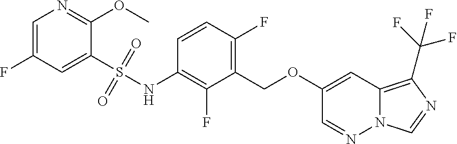 [SMILES: COc1ncc(F)cc1S(=O)(=O)Nc1ccc(F)c(COc2cnn3cnc(C(F)(F)F)c3c2)c1F] | 1 | [M + H]⁺: 534 |
| 84 | N-[2,4-difluoro-3- [([3-phenyl-1H- pyrazolo[3,4- b]pyridin-5- yl]oxy)methyl] phenyl]-5-fluoro-2- methoxypyridine-3- sulfonamide | 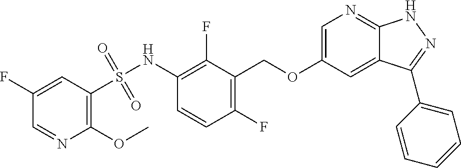 [SMILES: COc1ncc(F)cc1S(=O)(=O)Nc1ccc(F)c(COc2cnc3[nH]nc(-c4ccccc4)c3c2)c1F] | 1 | [M + H]⁺: 542 |
| 85 | N-[2,4-difluoro-3- [([3-methyl-4-phenyl- 1H-pyrazolo[3,4-b] pyridin-5- yl]oxy)methyl] phenyl]-5-fluoro-2- methoxypyridine-3- sulfonamide | 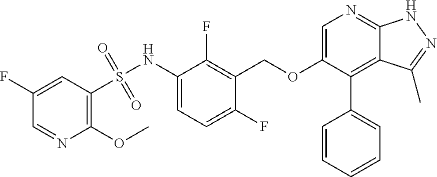 [SMILES: COc1ncc(F)cc1S(=O)(=O)Nc1ccc(F)c(COc2cnc3[nH]nc(C)c3c2-c2ccccc2)c1F] | 1 | [M + H]⁺: 556 |
| 86 | N-(3-(((4- cyclopropyl-3- methyl-1H- pyrazolo[3,4- b]pyridin-5- yl)oxy)methyl)-2,4- difluorophenyl)-5- fluoro-2- methoxypyridine-3- sulfonamide | 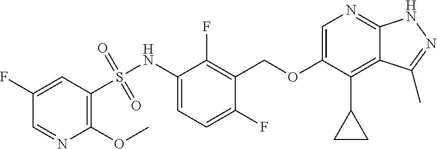 [SMILES: COc1ncc(F)cc1S(=O)(=O)Nc1ccc(F)c(COc2cnc3[nH]nc(C)c3c2C2CC2)c1F] | 1 | [M + H]⁺: 520 |
| 87 | N-(2,4-difluoro-3- (((3-methyl-4- (pyridin-4-yl)-1H- pyrazolo[3,4- b]pyridin-5- yl)oxy)methyl) phenyl)-5-fluoro-2- methoxypyridine-3- sulfonamide | 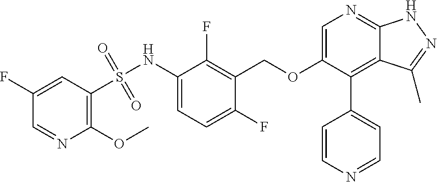 [SMILES: COc1ncc(F)cc1S(=O)(=O)Nc1ccc(F)c(COc2cnc3[nH]nc(C)c3c2-c2ccncc2)c1F] | | [M + H]⁺: 557 |
| 88 | N-(2,4-difluoro-3- (((4-isopropyl-3- methyl-1H- pyrazolo[3,4- b]pyridin-5- yl)oxy)methyl)phenyl)- 5-fluoro-2- methoxypyridine-3- sulfonamide | 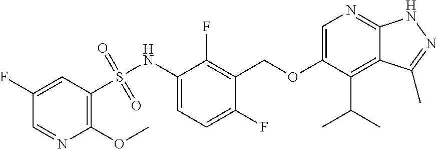 [SMILES: COc1ncc(F)cc1S(=O)(=O)Nc1ccc(F)c(COc2cnc3[nH]nc(C)c3c2C(C)C)c1F] | | [M + H]⁺: 522 |
| 89 | N-(3-(((4-benzyl-3- methyl-1H- pyrazolo[3,4- b]pyridin-5- yl)oxy)methyl)-2,4- difluorophenyl)-5- fluoro-2- methoxypyridine-3- sulfonamide | 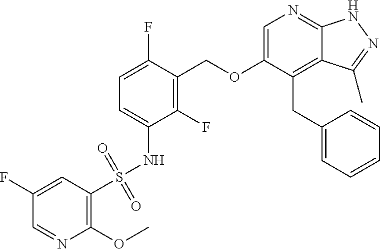 [SMILES: COc1ncc(F)cc1S(=O)(=O)Nc1ccc(F)c(COc2cnc3[nH]nc(C)c3c2Cc2ccccc2)c1F] | | [M + H]⁺: 570 |
| 90 | N-(2,4-difluoro-3- (((3-methyl-4- (pyridin-2-yl)-1H- pyrazolo[3,4- b]pyridin-5- yl)oxy)methyl)phenyl)- 5-fluoro-2- methoxypyridine-3- sulfonamide | 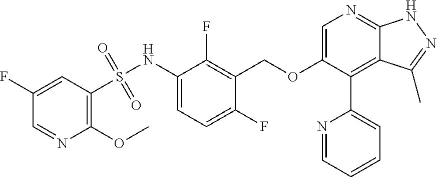 [SMILES: COc1ncc(F)cc1S(=O)(=O)Nc1ccc(F)c(COc2cnc3[nH]nc(C)c3c2-c2ccccn2)c1F] | | [M + H]⁺: 557 |
| 91 | N-(2,4-difluoro-3- (((3-methyl-4- (pyridin-3-yl)-1H- pyrazolo[3,4- b]pyridin-5- yl)oxy)methyl)phenyl)- 5-fluoro-2- methoxypyridine-3- sulfonamide | 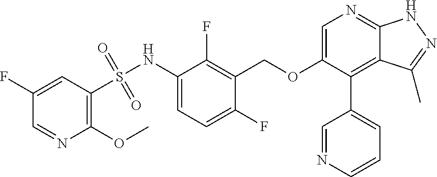 [SMILES: COc1ncc(F)cc1S(=O)(=O)Nc1ccc(F)c(COc2cnc3[nH]nc(C)c3c2-c2cccnc2)c1F] | | [M + H]⁺: 557 |
| 92 | N-(3-(((4- cyclopentyl-3- methyl-1H- pyrazolo[3,4- b]pyridin-5- yl)oxy)methyl)-2,4- difluorophenyl)-5- fluoro-2- methoxypyridine-3- sulfonamide | 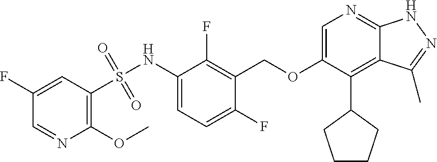 [SMILES: COc1ncc(F)cc1S(=O)(=O)Nc1ccc(F)c(COc2cnc3[nH]nc(C)c3c2C2CCCC2)c1F] | 1 | [M + H]⁺: 548 |
| 93 | N-(2,4-difluoro-3- (((3-methyl-4-(1- methylpiperidin-4- yl)-1H-pyrazolo[3,4- b]pyridin-5- yl)oxy)methyl)phenyl)- 5-fluoro-2- methoxypyridine-3- sulfonamide | 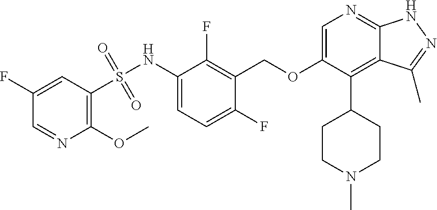 [SMILES: COc1ncc(F)cc1S(=O)(=O)Nc1ccc(F)c(COc2cnc3[nH]nc(C)c3c2C2CCN(C)CC2)c1F] | 1 | [M + H]⁺: 577 |
| 94 | N-(2,4-difluoro-3- (((3-methyl-4- (piperidin-1-yl)-1H- pyrazolo[3,4- b]pyridin-5- yl)oxy)methyl)phenyl)- 5-fluoro-2- methoxypyridine-3- sulfonamide | 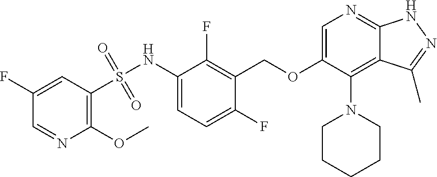 [SMILES: COc1ncc(F)cc1S(=O)(=O)Nc1ccc(F)c(COc2cnc3[nH]nc(C)c3c2N2CCCCC2)c1F] | 1 | [M + H]⁺: 563 |

### Example 97—Biochemical Assay for Modulation (Inhibition/Activation) of GCN2

Exemplary compounds from the above Examples were tested for ability to inhibit GCN2 activity using a time resolved fluorescence energy transfer (TR-FRET) assay. Assay procedures and results are described below for Examples 12-105.

### Part I—Procedures for TR-FRET Assay

GCN2 protein was obtained from Carma Biosciences (cat #05-153). The protein was diluted in assay buffer (ThermoFisher Scientific, #PV6135), 2 mM dithiothreitol (DTT), to obtain a final concentration of 2 nM and 5 μL was plated in a 384-well white assay plate. Test compounds were serially diluted to 11 concentrations by 3-fold dilution in DMS0 and 10 nL of stock was plated into 384 well white assay plate. DMS0 was used as a vehicle control. GFP-eIF2α protein was obtained from ThermoFisher (cat #PV4809). The protein was diluted in assay buffer to a 2× concentration of 200 nM along with 300 μM ATP (final concentration of 100 nM GFP-eIF2α and 150 μM ATP) in the presence of 2 mM DTT and a L aliquot was added to each well containing the GCN2 protein and test compound. The plate was incubated in the dark at 25° C. for 1.5 hours, shaking at 1250 rpm. Tb-anti P-eIF2α (ThermoFisher cat #PV4815) was diluted to a to a concentration of 1 nM in TR-FRET Dilution Buffer (ThermoFisher cat #PV3574). 10 μL of the Tb-anti P-eIF2a solution was added to the TR-FRET reaction. The plate was incubated in the dark for 2 h at 25° C. shaking at 600 rpm. The FRET signal from the plate was read on a Envision (PerkinElmer) plate reader:
Label 1: Excitation: 340 nm, bandwidth 30 nm; Emission: 495 nm, bandwidth 10 nm. Lag time: 100 μsec. Integration time: 400 μsec. Flashes: 30.
Label 2: Excitation: 340 nm, bandwidth 30 nm; Emission: 520 nm, bandwidth 25 nm. Lag time: 100 μsec. Integration time: 400 μsec. Flashes: 30

The data were analyzed using GraphPad Prism employing a 4-parameter sigmoidal curve fit.

### Part II—Results

Experimental results are provided in Table 2 below. The symbol “++++” indicates an IC₅₀ less than 0.0500 μM. The symbol “+++” indicates an IC₅₀ in the range of 0.0500 μM to 0.5000 μM. The symbol “++” indicates an IC₅₀ in the range of greater than 0.5000 μM to 1.0000 μM. The symbol “+” indicates an IC₅₀ greater than 1.0000 μM. The symbol “N/A” indicates that no data was available.

TABLE 2

| Compound No. | IC₅₀/μM |
|---|---|
| 1 | ++++ |
| 2 | ++++ |
| 3 | +++ |
| 4 | ++++ |
| 5 | + |
| 6 | +++ |
| 7 | ++++ |
| 8 | ++++ |
| 9 | ++++ |
| 10 | ++++ |
| 11 | + |
| 12 | ++++ |
| 13 | + |
| 14 | ++++ |
| 15 | ++++ |
| 16 | +++ |
| 17 | ++++ |
| 18 | ++++ |
| 19 | + |
| 20 | +++ |
| 21 | ++++ |
| 22 | ++++ |
| 23 | +++ |
| 24 | ++++ |
| 25 | ++ |
| 26 | ++++ |
| 27 | ++++ |
| 28 | ++++ |
| 29 | + |
| 30 | ++++ |
| 31 | ++++ |
| 32 | ++++ |
| 33 | ++++ |
| 34 | ++++ |
| 35 | ++++ |
| 36 | +++ |
| 37 | +++ |
| 38 | ++++ |
| 39 | ++++ |
| 40 | ++++ |
| 41 | ++++ |
| 42 | ++++ |
| 43 | ++++ |
| 44 | ++++ |
| 45 | ++++ |
| 46 | ++++ |
| 47 | ++++ |
| 48 | +++ |
| 49 | +++ |
| 50 | ++++ |
| 51 | +++ |
| 52 | ++++ |
| 53 | ++++ |
| 54 | ++++ |
| 55 | +++ |
| 56 | ++++ |
| 57 | +++ |
| 58 | ++++ |
| 59 | +++ |
| 60 | ++++ |
| 61 | ++++ |
| 62 | +++ |
| 63 | +++ |
| 64 | ++++ |
| 65 | ++++ |
| 66 | ++++ |
| 67 | + |
| 68 | +++ |
| 69 | ++++ |
| 70 | ++++ |
| 71 | ++++ |
| 72 | ++++ |
| 73 | ++++ |
| 74 | ++++ |
| 75 | ++++ |
| 76 | ++++ |
| 77 | ++++ |
| 78 | ++++ |
| 79 | ++++ |
| 80 | ++++ |
| 81 | ++++ |
| 82 | ++++ |
| 83 | ++++ |
| 84 | ++++ |
| 85 | +++ |
| 86 | +++ |
| 87 | +++ |
| 88 | + |
| 89 | + |
| 90 | +++ |
| 91 | +++ |
| 92 | ++ |
| 93 | + |
| 94 | +++ |

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

1.-15. (canceled)

16. A compound represented by Formula (Ib): 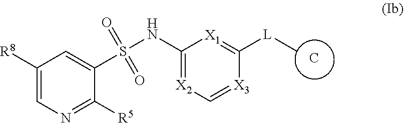
or a pharmaceutically acceptable salt thereof, wherein:
C is 5-10 membered heteroaryl or 7-10 membered heterocyclyl, wherein the 5-10 membered heteroaryl or 7-10 membered heterocyclyl may be optionally substituted on one or more available carbons by one, two, three, or more substituents each independently selected from R³; and wherein, if the 5-10 membered heteroaryl or 7-10 membered heterocyclyl contains a substitutable ring nitrogen atom, that ring nitrogen atom may be optionally substituted by C₁₋₆alkyl;
L is selected from the group consisting of C₁₋₆alkylene, C₁₋₆alkylene-O—, and C₁₋₆alkylene-N(R^{A})—;
R³ is independently, for each occurrence, selected from the group consisting of C₁₋₆alkyl, C₂₋₆alkynyl, C₃₋₆cycloalkyl, cyano, C₁₋₆alkoxyl, oxo, phenyl, —C(O)N(R^{A})(R^{B}), —N(R^{A})(R^{B}), 6 membered heterocyclyl, and 5-6 membered heteroaryl, wherein the C₁₋₆alkyl may each individually be optionally substituted on one or more available carbons by one, two, three, or more substituents each independently selected from halogen and phenyl; and wherein if the 6 membered heterocyclyl contains a substitutable ring nitrogen atom, that ring nitrogen atom may be optionally substituted by C₁₋₆alkyl; or
two R³ substituents together with the carbon to which they are attached form C₃₋₆cycloalkyl;
X¹ is selected from CR⁶ and N;
X² is selected from CH and N;
X³ is selected from CR⁷ and N;
R⁴ is selected from halogen and cyano;
R⁵ is selected from C₁₋₆alkyl and C₁₋₆alkoxyl;
R⁶ is selected from halogen and cyano;
R⁷ is halogen; and
R^{A} and R^{B} are independently, for each occurrence, hydrogen or C₁₋₆alkyl.

17. The compound of claim 16, wherein R⁴ is selected from the group consisting of chloro, fluoro, and cyano.

18. The compound of claim 16, wherein R⁵ is CH₃ or —O—CH₃.

19. The compound of claim 16, wherein X¹ is CR⁶; X² is CH; and X³ is CR⁷.

20. The compound of claim 19, wherein R⁶ is selected from the group consisting of chloro, fluoro, and cyano.

21. The compound of claim 19, wherein R⁷ is chloro or fluoro.

22. The compound of claim 16, wherein X¹ is CR⁶; X² is N; and X³ is CR⁷.

23. The compound of claim 22, wherein R⁶ is chloro or fluoro.

24. The compound of claim 22, wherein R⁷ is fluoro.

25. The compound of claim 16, wherein X¹ is CR⁶; X² is CH; and X³ is N.

26. The compound of claim 25, wherein R⁶ is fluoro.

27. The compound of claim 16, wherein X¹ is N; X² is CH; and X³ is CR⁷.

28. The compound of claim 27, wherein R⁷ is fluoro.

29. The compound of claim 16, wherein L is selected from the group consisting of —CH₂—CH₂—, —C(CH₃)(H)—O—, —CH₂—O—, —CH₂—N(H)—, and —C(CH₃)(H)—N(H)—.

30. (canceled)

31. The compound of claim 16, wherein C is selected from the group consisting of 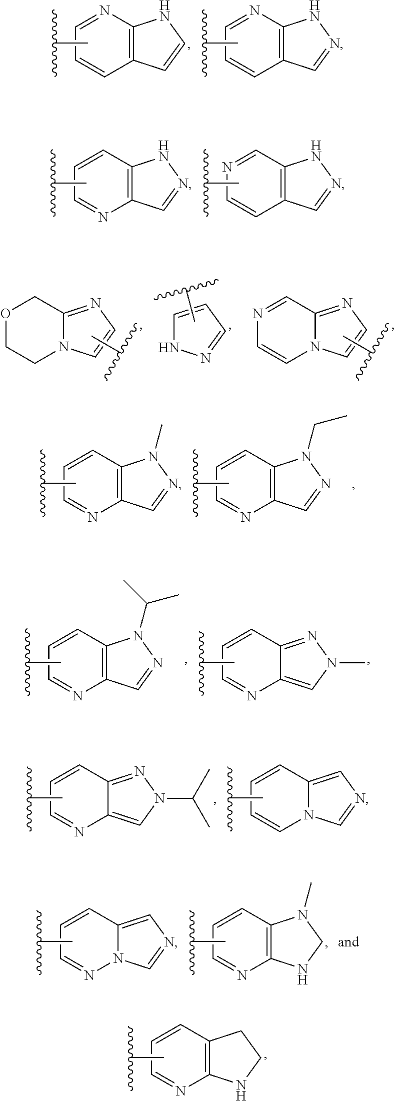
wherein C may be optionally substituted on one or more available carbons by one, two, three, or more R³ substituents independently selected from the group consisting of C₁₋₆alkyl, C₂₋₆alkynyl, C₃₋₆cycloalkyl, cyano, C₁₋₆alkoxyl, oxo, phenyl, —C(O)N(R^{A})(R^{B}), —N(R^{A})(R^{B}), 5-6 membered heterocyclyl, and 5-6 membered heteroaryl, wherein
the C₁₋₆alkyl may be optionally substituted on one or more available carbons by one, two, three, or more substituents each independently selected from halogen and phenyl;
if the 5-6 membered heterocyclyl contains a substitutable ring nitrogen atom, that ring nitrogen atom may be optionally substituted by C₁₋₆alkyl; and
R^{A} and R^{B} are independently, for each occurrence, hydrogen or C₁₋₆alkyl.

32. The compound of claim 16, wherein C is selected from the group consisting of 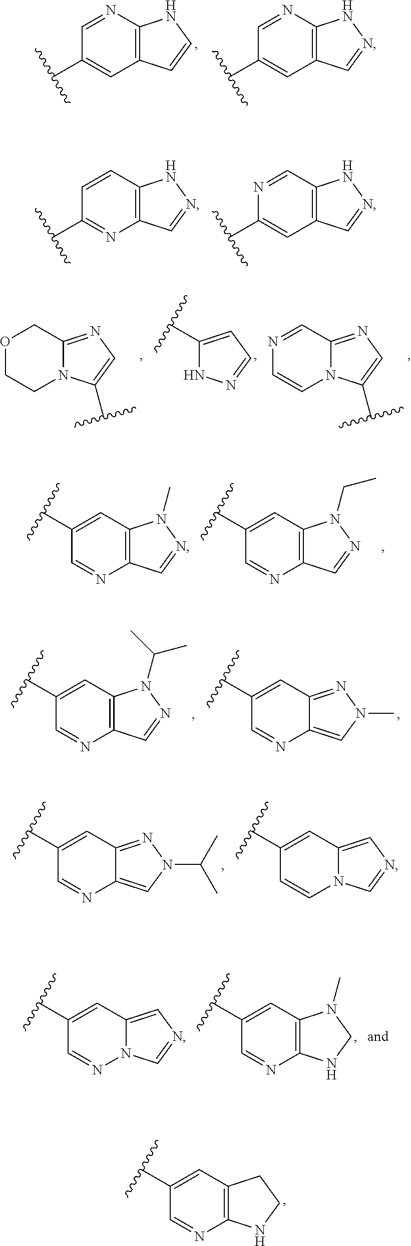
wherein C may be optionally substituted on one or more available carbons by one, two, three, or more independent R³ substituents selected from the group consisting of C₁₋₆alkyl, C₂₋₆alkynyl, C₃₋₆cycloalkyl, cyano, C₁₋₆alkoxyl, oxo, phenyl, —C(O)N(R^{A})(R^{B}), —N(R^{A})(R^{B}), 5-6 membered heterocyclyl, and 5-6 membered heteroaryl, wherein
the C₁₋₆alkyl may be optionally substituted on one or more available carbons by one, two, three, or more substituents each independently selected from halogen and phenyl;
if the 5-6 membered heterocyclyl contains a substitutable ring nitrogen atom, that ring nitrogen atom may be optionally substituted by C₁₋₆alkyl; and
R^{A} and R^{B} are independently, for each occurrence, hydrogen or C₁₋₆alkyl.

33.-43. (canceled)

44. The compound of claim 16, wherein C is selected from the group consisting of

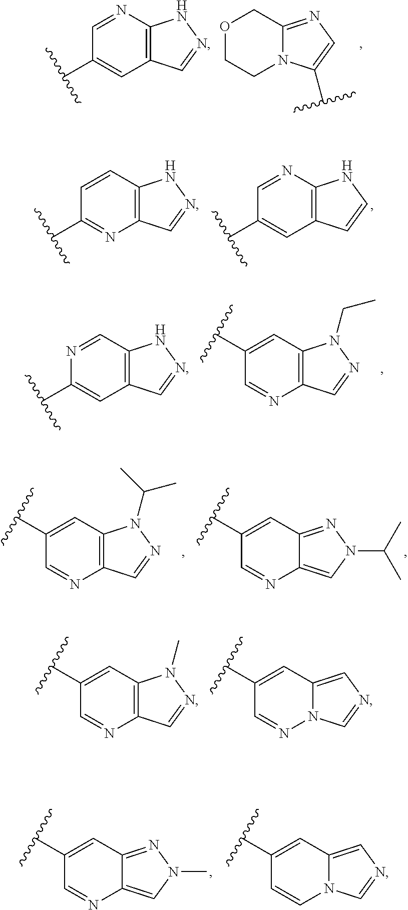

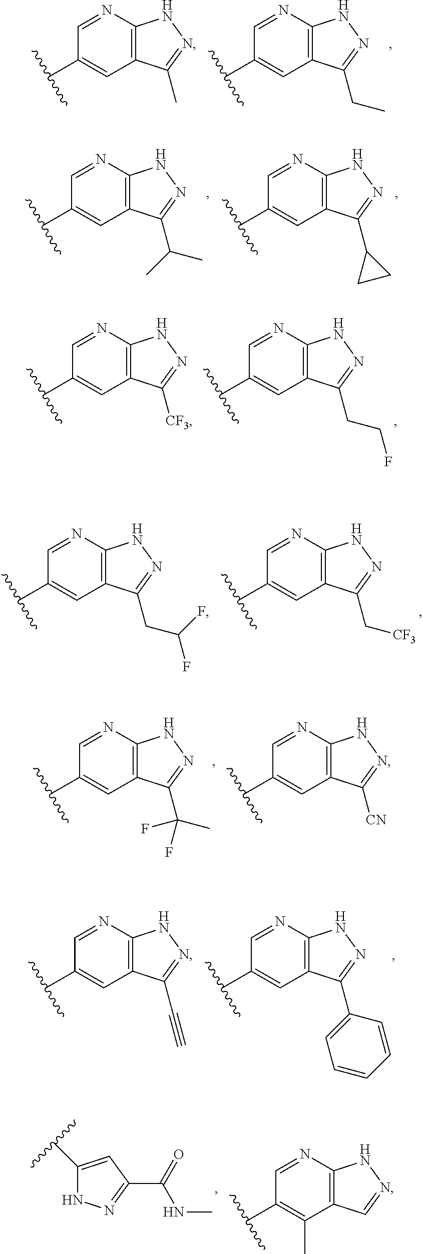

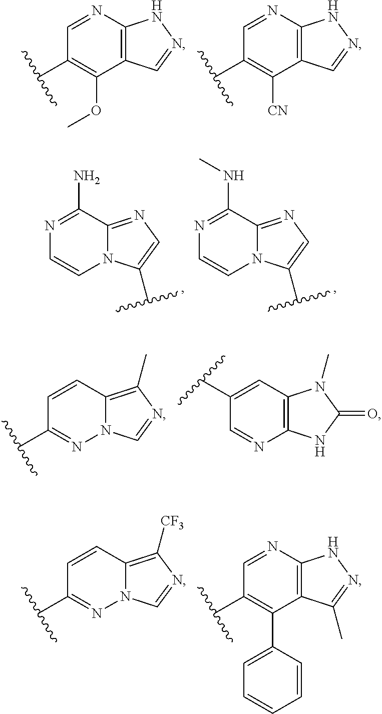

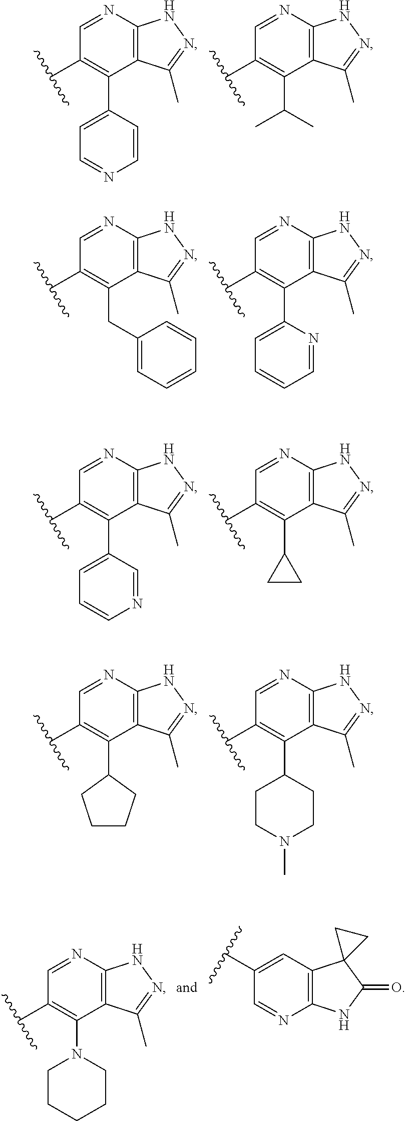

45. The compound of claim 16 selected from the group consisting of:
5-chloro-N-[2,4-difluoro-3-([1H-pyrazolo[3,4-b]pyridin-5-yloxy]methyl)phenyl]-2-methoxypyridine-3-sulfonamide;
N-[2,4-difluoro-3-[([3-methyl-1H-pyrazolo[3,4-b]pyridin-5-yl]oxy)methyl]phenyl]-5-fluoro-2-methoxypyridine-3-sulfonamide:
5-chloro-N-[2,4-difluoro-3-([1H-pyrazolo[3,4-b]pyridin-5-ylamino]methyl)phenyl]-2-methoxypyridine-3-sulfonamide:
5-chloro-N-(2,4-difluoro-3-[1H-pyrazolo[3,4-b]pyridin-5-ylmethoxy]phenyl)-2-methoxypyridine-3-sulfonamide:
5-chloro-N-[2,4-difluoro-3-(2-[5H,6H,8H-imidazo[2,1-c][1,4]oxazin-3-yl]ethyl)phenyl]-2-methoxypyridine-3-sulfonamide:
5-chloro-N-[2,4-difluoro-3-([1H-pyrazolo[3,4-b]pyridin-5-ylmethyl]amino)phenyl]-2-methoxypyridine-3-sulfonamide:
5-chloro-N-[2,4-difluoro-3-(2-[1H-pyrazolo[3,4-b]pyridin-5-yl]ethyl)phenyl]-2-methoxypyridine-3-sulfonamide;
5-[2-[3-(5-chloro-2-methoxypyridine-3-sulfonamido)-2,6-difluorophenyl]ethyl]-N-methyl-1H-pyrazole-3-carboxamide;
N-(3-(2-(1H-pyrazolo[3,4-b]pyridin-5-yl)ethyl)-2,4-difluorophenyl)-5-fluoro-2-methylpyridine-3-sulfonamide;
5-cyano-N-[2,4-difluoro-3-(2-[1H-pyrazolo[3,4-b]pyridin-5-yl]ethyl)phenyl]-2-methoxypyridine-3-sulfonamide:
5-chloro-N-[2,4-difluoro-3-(2-[1H-pyrazolo[4,3-b]pyridin-5-yl]ethyl)phenyl]-2-methoxypyridine-3-sulfonamide:
5-chloro-N-[2,4-difluoro-3-(2-[1H-pyrrolo[2,3-b]pyridin-5-yl]ethyl)phenyl]-2-methoxypyridine-3-sulfonamide:
5-chloro-N-[2,4-difluoro-3-(2-[1H-pyrazolo[3,4-c]pyridin-5-yl]ethyl)phenyl]-2-methoxypyridine-3-sulfonamide:
5-chloro-N-[2,4-difluoro-3-[([3-methyl-1H-pyrazolo[3,4-b]pyridin-5-yl]oxy)methyl]phenyl]-2-methoxypyridine-3-sulfonamide;
N-[2,4-difluoro-3-(2-[1H-pyrazolo[3,4-b]pyridin-5-yl]ethyl)phenyl]-5-fluoro-2-methoxypyridine-3-sulfonamide:
5-chloro-N-[2,4-difluoro-3-(2-[2-methylpyrazolo[4,3-b]pyridin-6-yl]ethyl)phenyl]-2-methoxypyridine-3-sulfonamide:
5-chloro-N-[2,4-difluoro-3-(2-[1-methylpyrazolo[4,3-b]pyridin-6-yl]ethyl)phenyl]-2-methoxypyridine-3-sulfonamide:
5-chloro-N-[2,4-difluoro-3-(2-[imidazo[1,5-al pyridin-7-yl]ethyl)phenyl]-2-methoxypyridine-3-sulfonamide;
N-[2,4-difluoro-3-[([4-methyl-1H-pyrazolo[3,4-b]pyridin-5-yl]oxy)methyl]phenyl]-5-fluoro-2-methoxypyridine-3-sulfonamide;
N-[2,4-difluoro-3-([1H-pyrazolo[3,4-b]pyridin-5-yloxy]methyl)phenyl]-5-fluoro-2-methoxypyridine-3-sulfonamide:
5-chloro-N-[2,4-difluoro-3-[([4-methyl-1H-pyrazolo[3,4-b]pyridin-5-yl]oxy)methyl]phenyl]-2-methoxypyridine-3-sulfonamide;
N-[2,4-difluoro-3-[([4-methoxy-1H-pyrazolo[3,4-b]pyridin-5-yl]oxy)methyl]phenyl]-5-fluoro-2-methoxypyridine-3-sulfonamide:
5-chloro-N-[2,4-difluoro-3-[([4-methoxy-1H-pyrazolo[3,4-b]pyridin-5-yl]oxy)methyl]phenyl]-2-methoxypyridine-3-sulfonamide;
5-chloro-N-[2,4-difluoro-3-(1-[1H-pyrazolo[3,4-b]pyridin-5-yloxy]ethyl)phenyl]-2-methoxypyridine-3-sulfonamide;
5-cyano-N-[2,4-difluoro-3-([1H-pyrazolo[3,4-b]pyridin-5-yloxy]methyl)phenyl]-2-methoxypyridine-3-sulfonamide;
N-[3-[([4-cyano-1H-pyrazolo[3,4-b]pyridin-5-yl]oxy)methyl]-2,4-difluorophenyl]-5-fluoro-2-methoxypyridine-3-sulfonamide;
5-chloro-N-[3-[([4-cyano-1H-pyrazolo[3,4-b]pyridin-5-yl]oxy)methyl]-2,4-difluorophenyl]-2-methoxypyridine-3-sulfonamide;
5-chloro-N-[2,4-difluoro-3-(1-[1H-pyrazolo[3,4-b]pyridin-5-ylamino]ethyl)phenyl]-2-methoxypyridine-3-sulfonamide;
N-(3-(((1H-pyrazolo[3,4-b]pyridin-5-yl)oxy)methyl)-2,4-difluorophenyl)-5-fluoro-2-methylpyridine-3-sulfonamide;
5-chloro-N-(2,4-difluoro-3-(2-(8-(methylamino)imidazo[1,2-a]pyrazin-3-yl)ethyl)phenyl)-2-methoxypyridine-3-sulfonamide;
N-[3-(2-[8-aminoimidazo[1,2-a]pyrazin-3-yl]ethyl)-2,4-difluorophenyl]-5-chloro-2-methoxypyridine-3-sulfonamide;
5-chloro-N-[3-(2-[1-ethylpyrazolo[4,3-b]pyridin-6-yl]ethyl)-2,4-difluorophenyl]-2-methoxypyridine-3-sulfonamide;
N-[3-(2-[1-ethylpyrazolo[4,3-b]pyridin-6-yl]ethyl)-2,4-difluorophenyl]-5-fluoro-2-methoxypyridine-3-sulfonamide;
5-chloro-N-[2,4-difluoro-3-(2-[1-isopropylpyrazolo[4,3-b]pyridin-6-yl]ethyl)phenyl]-2-methoxypyridine-3-sulfonamide;
N-[2,4-difluoro-3-(2-[1-isopropylpyrazolo[4,3-b]pyridin-6-yl]ethyl)phenyl]-5-fluoro-2-methoxypyridine-3-sulfonamide;
N-[2,4-difluoro-3-(2-[2-isopropylpyrazolo[4,3-b]pyridin-6-yl]ethyl)phenyl]-5-fluoro-2-methoxypyridine-3-sulfonamide;
N-[2,4-difluoro-3-[([3-methyl-1H-pyrazolo[3,4-b]pyridin-5-yl]oxy)methyl]phenyl]-5-fluoro-2-methylpyridine-3-sulfonamide;
N-[3-[([3-ethyl-1H-pyrazolo[3,4-b]pyridin-5-yl]oxy)methyl]-2,4-difluorophenyl]-5-fluoro-2-methoxypyridine-3-sulfonamide;
5-chloro-N-[3-[([3-ethyl-1H-pyrazolo[3,4-b]pyridin-5-yl]oxy)methyl]-2,4-difluorophenyl]-2-methoxypyridine-3-sulfonamide;
5-cyano-N-[3-[([3-ethyl-1H-pyrazolo[3,4-b]pyridin-5-yl]oxy)methyl]-2,4-difluorophenyl]-2-methoxypyridine-3-sulfonamide;
5-chloro-N-[2,4-difluoro-3-[(1-methylpyrazolo[4,3-b]pyridin-6-yl]oxy)methyl]phenyl]-2-methoxypyridine-3-sulfonamide;
N-[2,4-difluoro-3-[([1-methylpyrazolo[4,3-b]pyridin-6-yl]oxy)methyl]phenyl]-5-fluoro-2-methoxypyridine-3-sulfonamide;
N-[2,4-difluoro-3-([[3-(trifluoromethyl)-1H-pyrazolo[3,4-b]pyridin-5-yl]oxy]methyl)phenyl]-5-fluoro-2-methoxypyridine-3-sulfonamide;
N-[2,4-difluoro-3-([3-methyl-1H-pyrazolo[3,4-b]pyridin-5-yl]methoxy)phenyl]-5-fluoro-2-methoxypyridine-3-sulfonamide;
5-chloro-N-[2,4-difluoro-3-([3-methyl-1H-pyrazolo[3,4-b]pyridin-5-yl]methoxy)phenyl]-2-methoxypyridine-3-sulfonamide;
N-[3-[([3-ethyl-1H-pyrazolo[3,4-b]pyridin-5-yl]oxy)methyl]-2,4-difluorophenyl]-5-fluoro-2-methylpyridine-3-sulfonamide;
5-cyano-N-(2,4-difluoro-3-((3-methyl-1H-pyrazolo[3,4-b]pyridin-5-yl)methoxy)phenyl)-2-methoxypyridine-3-sulfonamide;
5-cyano-N-[2,4-difluoro-3-[([3-methyl-1H-pyrazolo[3,4-b]pyridin-5-yl]methyl)amino]phenyl]-2-methoxypyridine-3-sulfonamide;
5-cyano-N-[2,4-difluoro-3-[([3-methyl-1H-pyrazolo[3,4-b]pyridin-5-yl]oxy)methyl]phenyl]-2-methoxypyridine-3-sulfonamide;
N-[2,4-difluoro-3-([3-methyl-1H-pyrazolo[3,4-b]pyridin-5-yl]methoxy)phenyl]-5-fluoro-2-methylpyridine-3-sulfonamide;
N-[2,4-difluoro-3-[([3-isopropyl-1H-pyrazolo[3,4-b]pyridin-5-yl]oxy)methyl]phenyl]-5-fluoro-2-methoxypyridine-3-sulfonamide;
5-chloro-N-[2,4-difluoro-3-[([3-isopropyl-1H-pyrazolo[3,4-b]pyridin-5-yl]oxy)methyl]phenyl]-2-methoxypyridine-3-sulfonamide;
N-[2,4-difluoro-3-[([3-isopropyl-1H-pyrazolo[3,4-b]pyridin-5-yl]oxy)methyl]phenyl]-5-fluoro-2-methylpyridine-3-sulfonamide;
5-cyano-N-[2,4-difluoro-3-[([3-isopropyl-1H-pyrazolo[3,4-b]pyridin-5-yl]oxy)methyl]phenyl]-2-methoxypyridine-3-sulfonamide;
N-[3,5-difluoro-4-[([3-methyl-1H-pyrazolo[3,4-b]pyridin-5-yl]oxy)methyl]pyridin-2-yl]-5-fluoro-2-methoxypyridine-3-sulfonamide;
N-[2,4-difluoro-3-[([3-methyl-1H-pyrazolo[3,4-b]pyridin-5-yl]methyl)amino]phenyl]-5-fluoro-2-methoxypyridine-3-sulfonamide:
5-chloro-N-[2,4-difluoro-3-[([3-methyl-1H-pyrazolo[3,4-b]pyridin-5-yl]methyl)amino]phenyl]-2-methoxypyridine-3-sulfonamide;
N-[2,4-difluoro-3-[([3-methyl-1H-pyrazolo[3,4-b]pyridin-5-yl]methyl)amino]phenyl]-5-fluoro-2-methylpyridine-3-sulfonamide:
5-chloro-N-(2,4-difluoro-3-((imidazo[1,5-b]pyridazin-3-yloxy)methyl)phenyl)-2-methoxypyridine-3-sulfonamide;
N-[2-chloro-4-fluoro-3-[([3-methyl-1H-pyrazolo[3,4-b]pyridin-5-yl]oxy)methyl]phenyl]-5-fluoro-2-methoxypyridine-3-sulfonamide;
N-[2-cyano-4-fluoro-3-[([3-methyl-1H-pyrazolo[3,4-b]pyridin-5-yl]oxy)methyl]phenyl]-5-fluoro-2-methoxypyridine-3-sulfonamide:
5-fluoro-N-[3-fluoro-2-[([3-methyl-1H-pyrazolo[3,4-b]pyridin-5-yl]oxy)methyl]pyridin-4-yl]-2-methoxypyridine-3-sulfonamide:
5-chloro-N-[3-fluoro-2-[([3-methyl-1H-pyrazolo[3,4-b]pyridin-5-yl]oxy)methyl]pyridin-4-yl]-2-methoxypyridine-3-sulfonamide:
5-chloro-N-[3,5-difluoro-4-[([3-methyl-1H-pyrazolo[3,4-b]pyridin-5-yl]oxy)methyl]pyridin-2-yl]-2-methoxypyridine-3-sulfonamide;
N-[3-chloro-5-fluoro-4-[([3-methyl-1H-pyrazolo[3,4-b]pyridin-5-yl]oxy)methyl]pyridin-2-yl]-5-fluoro-2-methoxypyridine-3-sulfonamide:
5-fluoro-N-[5-fluoro-6-[([3-methyl-1H-pyrazolo[3,4-b]pyridin-5-yl]oxy)methyl]pyridine-2-yl]-2-methoxypyridine-3-sulfonamide:
5-chloro-N-[5-fluoro-6-[([3-methyl-1H-pyrazolo[3,4-b]pyridin-5-yl]oxy)methyl]pyridin-2-yl]-2-methoxypyridine-3-sulfonamide;
N-[3-[([3-ethynyl-1H-pyrazolo[3,4-b]pyridin-5-yl]oxy)methyl]-2,4-difluorophenyl]-5-fluoro-2-methoxypyridine-3-sulfonamide;
N-(3-(((3-cyclopropyl-1H-pyrazolo[3,4-b]pyridin-5-yl)oxy)methyl)-2,4-difluorophenyl)-5-fluoro-2-methoxypyridine-3-sulfonamide;
N-[2,4-difluoro-3-[([1-methyl-2-oxo-3H-imidazo[4,5-b]pyridin-6-yl]oxy)methyl]phenyl]-5-fluoro-2-methoxypyridine-3-sulfonamide;
N-(2,4-difluoro-3-(((2′-oxo-1′,2′-dihydrospiro[cyclopropane-1,3′-pyrrolo[2,3-b]pyridin]-5′-yl)oxy)methyl)phenyl)-5-fluoro-2-methoxypyridine-3-sulfonamide;
N-(3-(((3-cyano-1H-pyrazolo[3,4-b]pyridin-5-yl)oxy)methyl)-2,4-difluorophenyl)-5-fluoro-2-methoxypyridine-3-sulfonamide;
N-[2-chloro-4-fluoro-3-[([3-methyl-1H-pyrazolo[3,4-b]pyridin-5-yl]oxy)methyl]phenyl]-5-fluoro-2-methylpyridine-3-sulfonamide;
N-[3-([[3-(2,2-difluoroethyl)-1H-pyrazolo[3,4-b]pyridin-5-yl]oxy]methyl)-2,4-difluorophenyl]-5-fluoro-2-methoxypyridine-3-sulfonamide;
N-[2,4-difluoro-3-([[3-(2-fluoroethyl)-1H-pyrazolo[3,4-b]pyridin-5-yl]oxy]methyl)phenyl]-5-fluoro-2-methoxypyridine-3-sulfonamide;
N-(4-chloro-2-fluoro-3-(((3-methyl-1H-pyrazolo[3,4-b]pyridin-5-yl)oxy)methyl)phenyl)-5-fluoro-2-methylpyridine-3-sulfonamide;
N-(4-chloro-2-fluoro-3-(((3-methyl-1H-pyrazolo[3,4-b]pyridin-5-yl)oxy)methyl)phenyl)-5-fluoro-2-methoxypyridine-3-sulfonamide:
5-chloro-N-[3-chloro-5-fluoro-4-[([3-methyl-1H-pyrazolo[3,4-b]pyridin-5-yl]oxy)methyl]pyridin-2-yl]-2-methoxypyridine-3-sulfonamide;
N-[2,4-difluoro-3-([[3-(2,2,2-trifluoroethyl)-1H-pyrazolo[3,4-b]pyridin-5-yl]oxy]methyl)phenyl]-5-fluoro-2-methoxypyridine-3-sulfonamide;
N-[3-([[3-(1,1-difluoroethyl)-1H-pyrazolo[3,4-b]pyridin-5-yl]oxy]methyl)-2,4-difluorophenyl]-5-fluoro-2-methoxypyridine-3-sulfonamide;
N-[2,4-difluoro-3-[([5-methylimidazo[1,5-b]pyridazin-3-yl]oxy)methyl]phenyl]-5-fluoro-2-methoxypyridine-3-sulfonamide;
N-[2,4-difluoro-3-([[5-(trifluoromethyl)imidazo[1,5-b]pyridazin-3-yl]oxy]methyl)phenyl]-5-fluoro-2-methoxypyridine-3-sulfonamide;
N-[2,4-difluoro-3-[([3-phenyl-1H-pyrazolo[3,4-b]pyridin-5-yl]oxy)methyl]phenyl]-5-fluoro-2-methoxypyridine-3-sulfonamide;
N-[2,4-difluoro-3-[([3-methyl-4-phenyl-1H-pyrazolo[3,4-b]pyridin-5-yl]oxy)methyl]phenyl]-5-fluoro-2-methoxypyridine-3-sulfonamide;
N-[3-[([4-cyclopropyl-3-methyl-1H-pyrazolo[3,4-b]pyridin-5-yl]oxy)methyl]-2,4-difluorophenyl]-5-fluoro-2-methoxypyridine-3-sulfonamide;
N-[2,4-difluoro-3-([[3-methyl-4-(pyridin-4-yl)-1H-pyrazolo[3,4-b]pyridin-5-yl]oxy]methyl)phenyl]-5-fluoro-2-methoxypyridine-3-sulfonamide;
N-[2,4-difluoro-3-[([4-isopropyl-3-methyl-1H-pyrazolo[3,4-b]pyridin-5-yl]oxy)methyl]phenyl]-5-fluoro-2-methoxypyridine-3-sulfonamide;
N-[3-[([4-benzyl-3-methyl-1H-pyrazolo[3,4-b]pyridin-5-yl]oxy)methyl]-2,4-difluorophenyl]-5-fluoro-2-methoxypyridine-3-sulfonamide;
N-[2,4-difluoro-3-([[3-methyl-4-(pyridin-2-yl)-1H-pyrazolo[3,4-b]pyridin-5-yl]oxy]methyl)phenyl]-5-fluoro-2-methoxypyridine-3-sulfonamide;
N-[2,4-difluoro-3-([[3-methyl-4-(pyridin-3-yl)-1H-pyrazolo[3,4-b]pyridin-5-yl]oxy]methyl)phenyl]-5-fluoro-2-methoxypyridine-3-sulfonamide;
N-[3-[([4-cyclopentyl-3-methyl-1H-pyrazolo[3,4-b]pyridin-5-yl]oxy)methyl]-2,4-difluorophenyl]-5-fluoro-2-methoxypyridine-3-sulfonamide;
N-[2,4-difluoro-3-([[3-methyl-4-(1-methylpiperidin-4-yl)-1H-pyrazolo[3,4-b]pyridin-5-yl]oxy]methyl)phenyl]-5-fluoro-2-methoxypyridine-3-sulfonamide; and
N-[2,4-difluoro-3-([[3-methyl-4-(piperidin-1-yl)-1H-pyrazolo[3,4-b]pyridin-5-yl]oxy]methyl)phenyl]-5-fluoro-2-methoxypyridine-3-sulfonamide,
or a pharmaceutically acceptable salt thereof.

46. A pharmaceutical composition comprising a compound of claim 16 and a pharmaceutically acceptable carrier.

47. A method of treating cancer in a subject in need thereof, comprising administering to the subject a therapeutically effective amount of a compound of claim 16.

48.-54. (canceled)
